(19) United States
(12) Patent Application Publication
(10) Pub. No.: US 2026/0174876 A1
(43) Pub. Date: Jun. 25, 2026

(54) CB1 LIGAND CONJUGATED COMPOUNDS AND USES THEREOF

(71) Applicant: ADARx Pharmaceuticals, Inc., San Diego, CA (US)

(72) Inventors: Zhen Li, San Diego, CA (US); Rui Zhu, San Diego, CA (US); Mehdi Michel Djamel Numa, San Diego, CA (US); Chandramouli Chiruta, San Diego, CA (US)

(73) Assignee: ADARx Pharmaceuticals, Inc., San Diego, CA (US)

(21) Appl. No.: 18/842,673

(22) PCT Filed: Mar. 1, 2023
(86) PCT No.: PCT/US2023/063521

§ 371 Date: Aug. 29, 2024

Related U.S. Application Data
(60) Provisional application No. 63/315,472, filed on Mar. 1, 2022.
(60) Provisional application No. 63/327,345, filed on Apr. 4, 2022.

(51) Int. Cl. A61K 47/54; A61K 31/7088; A61K 47/55; C07F 9/6558

(52) U.S. Cl. CPC A61K 47/545; A61K 31/7088; A61K 47/543; A61K 47/548; A61K 47/55; C07F 9/65583

(57) ABSTRACT

Provided herein are CB₁ ligand-containing compounds, methods of delivering said compounds, and methods of treating diseases, disorders, and symptoms (e.g., central nervous system diseases, disorders, and symptoms) in a subject using said compounds.

### CROSS-REFERENCE TO RELATED APPLICATIONS

This application claims the benefit of priority under 35 U.S.C. § 119(e) of U.S. Provisional Application No. 63/315,472, filed Mar. 1, 2022; and U.S. Provisional Application No. 63/327,345, filed Apr. 4, 2022. The disclosure of each of the prior applications is considered part of and is incorporated by reference in its entirety in the disclosure of this application.

### BACKGROUND

In the use of compounds in therapeutic, prophylactic, or diagnostic applications, it is often desirable that the compounds be delivered to a specific location (for example, to desired cell(s)) to enhance the therapeutic or prophylactic effect or to be advantageous for diagnostic purposes. This is frequently the case when attempting to deliver a therapeutic compound in vivo. Further, being able to efficiently deliver a compound to a specific location can limit or potentially eliminate unintended consequences (such as off-target effects) that may be caused by administration of the compound. One strategy to facilitate delivery of a compound, such as a therapeutic, prophylactic, or diagnostic compound, to a desired location in vivo, is by linking or attaching the compound to a targeting ligand.

One class of compounds that can be targeted using targeting ligands are oligomeric compounds such as, for example, proteins, peptides, antibodies, and oligonucleotides. Oligomeric compounds that include nucleotide sequences (e.g., oligonucleotides) at least partially complementary to a target nucleic acid have been shown to alter the function and activity of the target both in vitro and in vivo. When delivered to a cell containing a target nucleic acid (such as mRNA or pre-mRNA), oligonucleotides have been shown to modulate the expression or activity of the target nucleic acid. In certain instances, the oligonucleotide can reduce the expression of the gene by inhibiting translation of the nucleic acid target and/or triggering the degradation of the target nucleic acid.

If the target nucleic acid is mRNA, one mechanism by which an oligonucleotide can modulate the expression of the mRNA target is through RNA interference. RNA interference is a biological process by which RNA or RNA-like molecules (such as chemically modified RNA molecules) are able to silence gene expression, at least in part, through the RNA-induced silencing Complex (RISC) pathway. Additionally, oligonucleotides can modulate the expression of a target nucleic acid, such as a target mRNA, through an RNase recruitment mechanism, microRNA mechanisms, occupancy-based mechanisms, and editing mechanisms. Oligonucleotides may be single-stranded or double-stranded. Oligonucleotides may comprise DNA, RNA, and RNA-like molecules, which can also include modified nucleosides including one or more modified sugars, modified nucleobases, and modified internucleoside linkages.

Another class of compounds that can be targeted using targeting ligands are small molecule compounds. The small molecule compounds (e.g., an organic compound having a molecular weight of ca. 1000 daltons or less) are typically shown to alter the function and/or activity of the target such that disease and/or disease symptoms are modulated or ameliorated or are typically useful as a diagnostic marker when localized to the target. More efficient delivery of a compound to a specific location can limit or potentially eliminate unintended consequences (such as off-target effects) that may be caused by administration of the compound and provide improved localization of a diagnostic compound.

### SUMMARY

Embodiments provided herein are directed to compounds (e.g., any of those delineated herein) and methods for targeting cells expressing Cannabinoid Receptor Type 1 (CB₁). Certain embodiments provided herein are directed to compounds and methods for delivering an agent to cells expressing CB₁. In certain embodiments, the cell is in the brain. In certain embodiments, the cell is in the frontal cortex. In certain embodiments, the cell is in the striatum. In certain embodiments, the cell is in the cerebellum. In certain embodiments, the cell is in the brain stem. In certain embodiments, the cell is in the hippocampus. In certain embodiments, the cell is in the spinal cord. In certain embodiments, the agent is a therapeutic compound. In certain embodiments, delivery of the agent is for the treatment of diseases, disorders, and symptoms in a subject. In certain embodiments, the agent is a diagnostic compound. In certain embodiments, a compound comprises a CB₁ ligand and one or more linker moieties for attachment to a therapeutic, prophylactic, or diagnostic agent. In certain embodiments, a compound comprises a CB₁ ligand, one or more linker moieties, and a therapeutic agent. In certain embodiments, the therapeutic agent is selected from a small molecule or an oligomeric compound. In certain embodiments, the oligomeric compound is a protein, peptide, antibody, oligonucleotide, or combination thereof. In certain embodiments, the CB₁ ligand is a CB₁ agonist. In certain embodiments, the CB₁ ligand is a CB₁ antagonist. In certain embodiments, the CB₁ ligand is a small molecule, an aptamer, a peptide, or an antibody. In certain embodiments, the CB₁ ligand is anandamide, or derivative thereof. In certain embodiments, the CB₁ ligand is (S)—N-(1-amino-3,3-dimethyl-1-oxobutan-2-yl)-1-(pent-4-en-1-yl)-1H-indazole-3-carboxamide, or a derivative thereof. In certain embodiments, the CB₁ ligand is any of those delineated herein, or a derivative or prodrug thereof.

In certain embodiments, contacting a cell expressing CB₁, such as a brain cell, with a compound provided herein, delivers the agent to the cell. In certain embodiments, contacting a cell expressing CB₁, such as a brain cell, with a compound provided herein, treats a disease, disorder, or symptom in a subject. In certain embodiments, a compound comprising a CB₁ ligand selectively or preferentially targets a cell expressing CB₁ compared to a cell not expressing CB₁. In certain embodiments, a compound comprising a CB₁ ligand selectively or preferentially targets a cell expressing CB₁ compared to a compound not comprising a CB₁ ligand.

Certain embodiments provided herein are directed to compounds and methods for modulating expression of a nucleic acid target in cells expressing CB₁. In certain embodiments, the cell is in the brain. In certain embodiments, the cell is in the frontal cortex. In certain embodiments, the cell is in the striatum. In certain embodiments, the cell is in the cerebellum. In certain embodiments, the cell is in the brain stem. In certain embodiments, the cell is in the hippocampus. In certain embodiments, the cell is in the spinal cord. In certain embodiments, contacting a cell expressing CB₁, such as a brain cell, with a compound provided herein, modulates the expression or activity of a nucleic acid target in the cell. In certain embodiments, a compound comprises a CB₁ ligand, one or more linker moieties, and an oligonucleotide.

It is understood that the embodiments provided herein with respect to preferred variable selections can be taken alone or in combination with one or more embodiments, or other preferred variable selections provided herein, as if each combination were explicitly listed herein.

In one aspect, the present disclosure provides compounds, and stereoisomers, tautomers, prodrugs, and salts thereof, comprising the structure of Formula (I′):

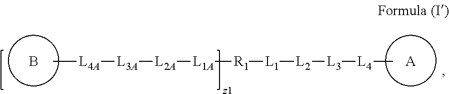

wherein:
each of

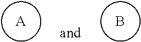

and is independently a cannabinoid receptor type 1 (CB₁) ligand;
each of L₁, L₂, L₃, L₄, L_{1A}, L_{2A}, L_{3A}, and L_{4A} is independently a linker, a bond, or absent;
R¹ comprises one or more oligonucleotides, protecting groups, small molecules, proteins, antibodies, and/or peptides; and
z1 is 0 or 1.

In some embodiments, the present disclosure provides compounds, and stereoisomers, tautomers, prodrugs, and salts thereof, comprising the structure of Formula (I″):

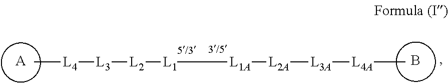

wherein:
[symbol] is an oligonucleotide; and

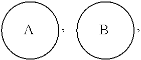

L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (I″-a):

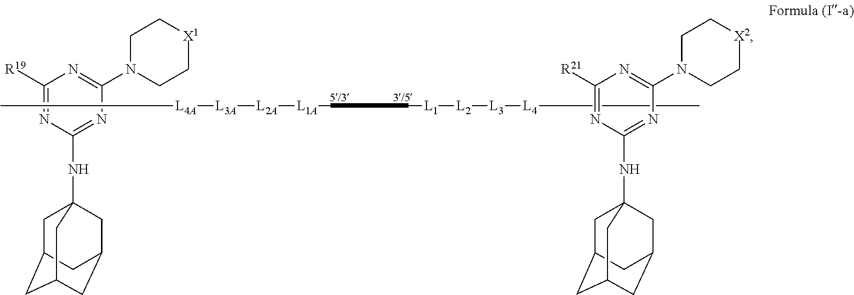

wherein:
X¹ is NR¹⁰ or CR¹¹R¹²;
X² is NR¹³ or CR¹⁴R¹⁵;
R¹⁰, R¹¹, R¹², R¹³, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D};
NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, —SOₙ₂₁R^{21A}, —SOᵥ₂₁NR^{21B}R^{21C}, NHNR^{21B}R^{21C}, ONR^{21B}R^{21C}, —NHC(O)NHNR^{21B}R^{21C}, —NHC(O)NR^{21B}R^{21C}, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D}, R^{21A}, R^{21B}, R^{21C} and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C}; and R^{21B} and R^{21C} substituents bonded to the same nitrogen atom may optionally be joined to form a substituted or unsubstituted heterocycloalkyl or substituted or unsubstituted heteroaryl;
n19 and n21 are each independently 0, 1, 2, 3, or 4;
v19 and v21 are each independently 1 or 2; and
L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (I″-a-1):

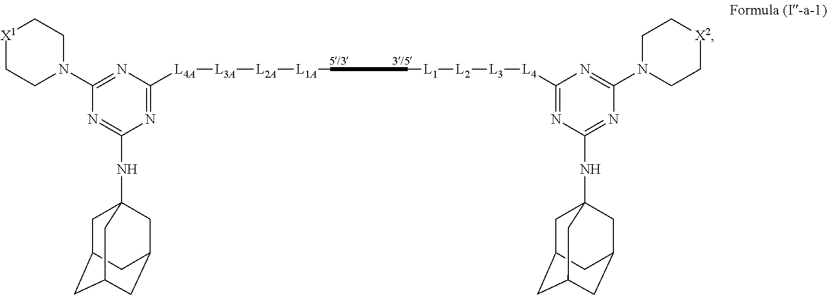

wherein X¹, X², L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (I″-a-2):

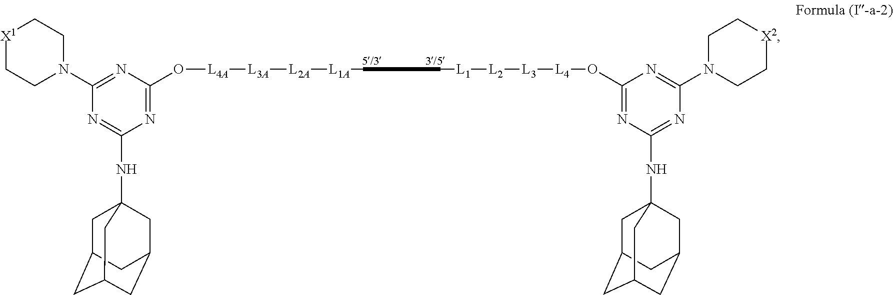

wherein X¹, X², L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (I″-b):

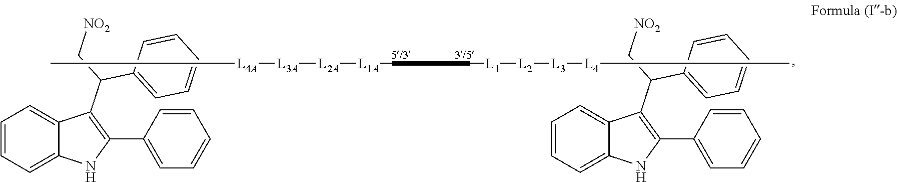

wherein L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (I″-b-1):

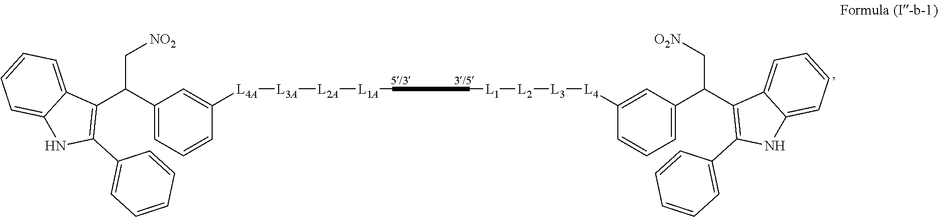

wherein L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (I″-c):

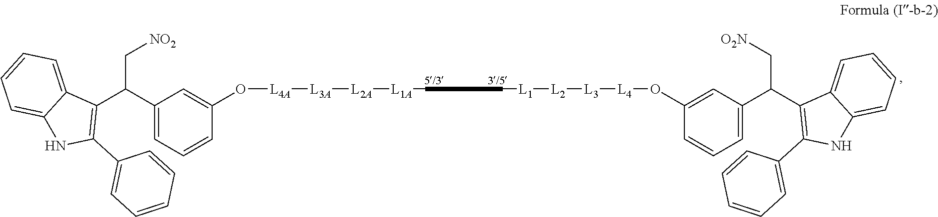

wherein L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (I″-c-1):

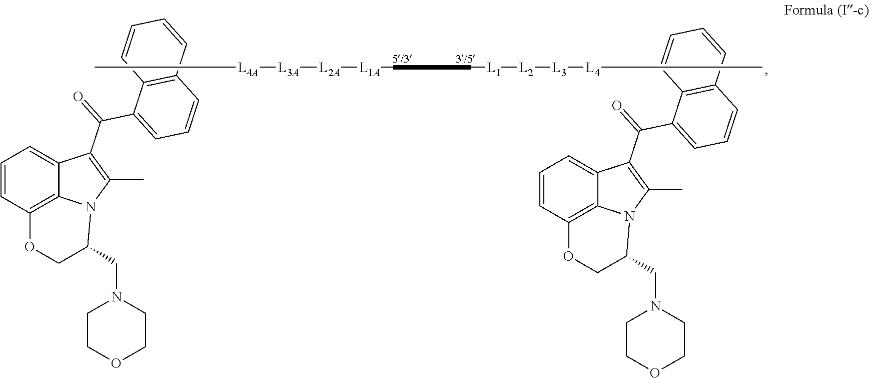

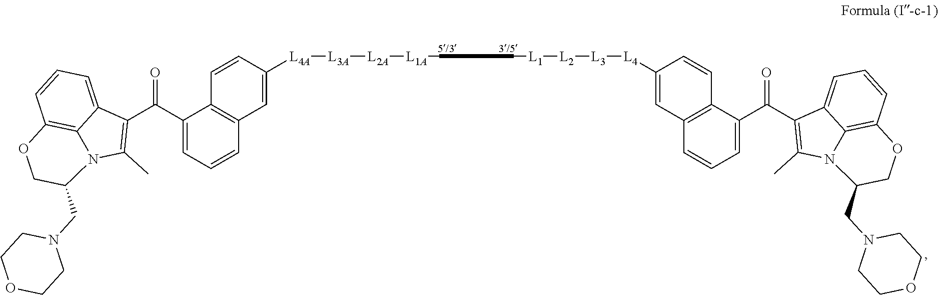

wherein L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (I″-c-2):

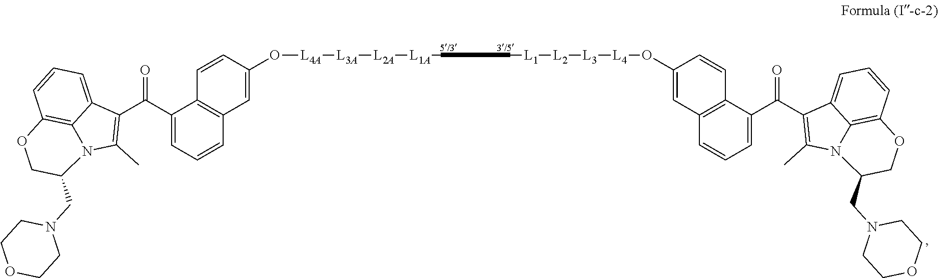

wherein L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (XII):

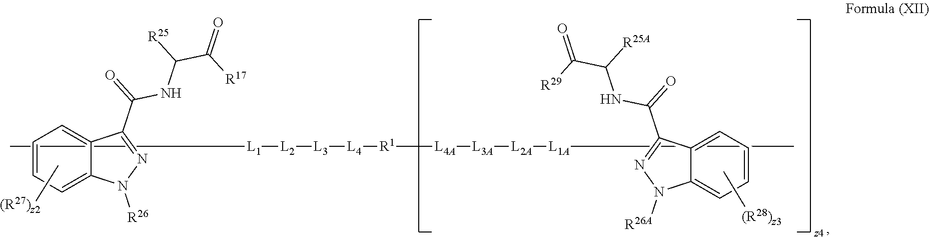

wherein:
L₁, L₂, L₃, L₄, L_{1A}, L_{2A}, L_{3A}, L_{4A}, R¹, and R¹⁷ are as defined herein;
z2 and z3 are each independently 0, 1, 2, 3, or 4;
z4 is 0 or 1;
R²⁵, R^{25A}, R²⁶, and R^{26A} are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁷ is hydrogen, —SOₙ₂₇R^{27A}, —SOᵥ₂₇NR^{27B}R^{27C}, —NHNR^{27B}R^{27C}, —ONR^{27B}R^{27C}, —NHC(O)NHNR^{27B}R^{27C}, —NHC(O)NR^{27B}R^{27C}, —NR^{27B}R^{27C}, —C(O)R^{27D}, —C(O)OR^{27D}, —C(O)NR^{27B}R^{27C}, —OR^{27A}, —NR^{27B}SO₂R^{27A}, —NR^{27B}C(O)R^{27D};
—NR^{27B}C(O)OR^{27D}, —NR^{27B}OR^{27D}, optionally substituted alkyl, optionally substituted heteroalkyl, optionally substituted cycloalkyl, optionally substituted heterocycloalkyl, optionally substituted aryl, or optionally substituted heteroaryl;
R^{27A}, R^{27B}, R^{27C}, and R^{27D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{27B} and R^{27C} substituents bonded to the same nitrogen atom may optionally be joined to form a substituted or unsubstituted heterocycloalkyl or substituted or unsubstituted heteroaryl;
R²⁸ is hydrogen, —SOₙ₂₈R^{28A}, —SOᵥ₂₈NR^{28B}R^{28C}, —NHNR^{28B}R^{28C}, —ONR^{28B}R^{28C}, —NHC(O)NHNR^{28B}R^{28C}, —NHC(O)NR^{28B}R^{28C}, —NR^{28B}R^{28C}, —C(O)R^{28D}, —C(O)OR^{28D}, —C(O)NR^{28B}R^{28C}, —OR^{28A}, —NR^{28B}SO₂R^{28A}, —NR^{28B}C(O)R^{28D};
—NR^{28B}C(O)OR^{28D}, —NR^{28B}OR^{28D}, optionally substituted alkyl, optionally substituted heteroalkyl, optionally substituted cycloalkyl, optionally substituted heterocycloalkyl, optionally substituted aryl, or optionally substituted heteroaryl;
R^{27A}, R^{27B}, R^{27C}, R^{27D}, R^{28A}, R^{28B}, R^{28C}, and R^{28D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{27B} and R^{27C}; R^{28B} and R^{28C} and substituents bonded to the same nitrogen atom may optionally be joined to form a substituted or unsubstituted heterocycloalkyl or substituted or unsubstituted heteroaryl;
n27 and n28 are each independently 0, 1, 2, 3, or 4; and
v27 and v28 are each independently 1 or 2.

In some embodiments, z4 is 0.

In some embodiments, the present disclosure provides compounds, and stereoisomers, tautomers, prodrugs, and salts thereof, wherein the CB₁ ligand comprises the structure of Formula (XII-a):

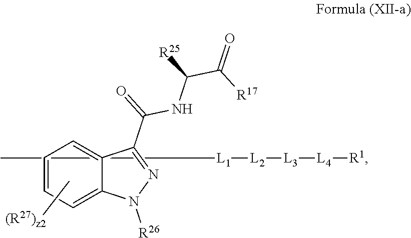

wherein L₁, L₂, L₃, L₄, R¹⁷, R²⁵, R²⁶, R²⁷, R¹, and z2 are as defined herein.

In some embodiments, the present disclosure provides compounds, and stereoisomers, tautomers, prodrugs, and salts thereof, comprising the structure of Formula (XII-a-1):

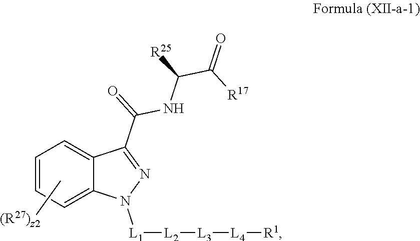

wherein L₁, L₂, L₃, L₄, R¹⁷, R²⁵, R²⁶, R²⁷, R¹, and z2 are as defined herein.

In some embodiments, the present disclosure provides compounds, and stereoisomers, tautomers, prodrugs, and salts thereof, comprising the structure of Formula (XII-b):

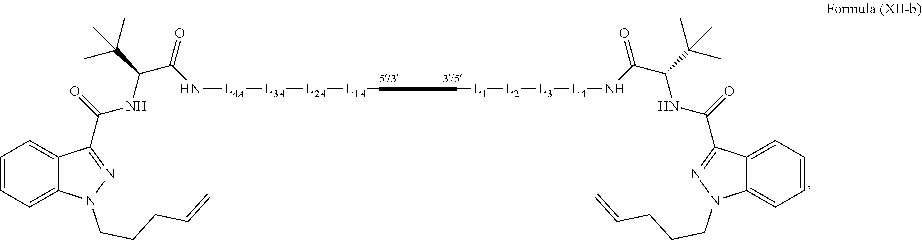

wherein L₁, L₂, L₃, L₄, L_{1A}, L_{2A}, L_{3A}, and L_{4A} are as defined herein.

In some embodiments, the present disclosure provides compounds, and stereoisomers, tautomers, prodrugs, and salts thereof, comprising the structure of Formula (XII-c):

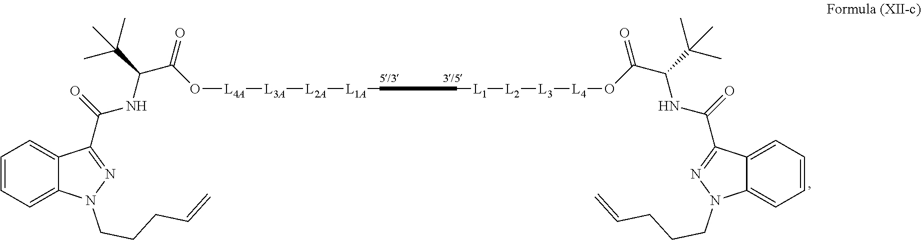

wherein L₁, L₂. L₃, L₄, L_{1A}, L_{2A}, L_{3A}, and L_{4A} are as defined herein.

In another aspect, the present disclosure provides compounds, and stereoisomers, tautomers, prodrugs, and salts thereof, comprising the structure of Formula (I):

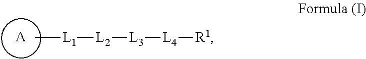

wherein

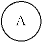

is a cannabinoid receptor type 1 (CB₁) ligand;
each of L₁, L₂, L₃, and L₄ is independently a linker, a bond, or absent; and
R¹ comprises one or more oligonucleotides, protecting groups, small molecules, proteins, antibodies, and/or peptides.

In some embodiments, the CB₁ ligand is a CB₁ agonist. In some embodiments, the CB₁ ligand is a CB₁ antagonist. In some embodiments, the CB₁ ligand is a selective ligand. In some embodiments, the CB₁ ligand is a non-selective ligand. In certain embodiments, the CB₁ ligand is selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

In some embodiments, the present disclosure provides compounds, and stereoisomers, tautomers, prodrugs, and salts thereof, wherein the CB₁ ligand comprises the structure of Formula (II′):

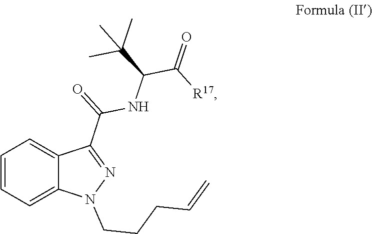

wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D};
—NR^{17B}C(O)OR^{7D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, and R^{17D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl;
n17 is 0, 1, 2, 3, or 4; and
v17 is 1 or 2.

In some embodiments, R¹⁷ is —NR^{17B}R^{17C}, —C(O)R^{17D}, or —C(O)OR^{17D}. In some embodiments, R^{17B} and R^{17C} are each independently hydrogen, optionally substituted alkyl, or optionally substituted heteroalkyl.

In certain embodiments, the CB₁ ligand comprises the structure

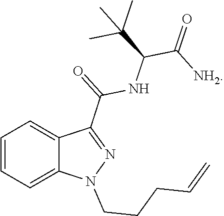 [SMILES: C=CCCCn1nc(C(=O)N[C@H](C(N)=O)C(C)(C)C)c2ccccc21]

In some embodiments, the present disclosure provides compounds, and stereoisomers, tautomers, prodrugs, and salts thereof, comprising the structure of Formula (II):

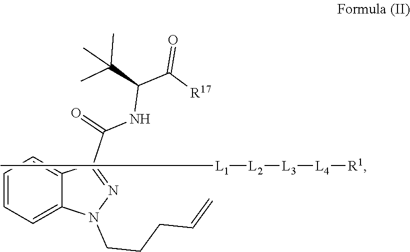

wherein R¹, R¹⁷, L₁, L₂, L₃, and L₄ are as defined herein.

In some embodiments, the present disclosure provides compounds, and stereoisomers, tautomers, prodrugs, and salts thereof, comprising the structure of Formula (II-a):

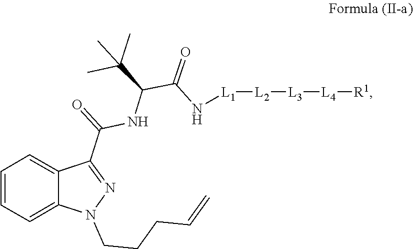

wherein R¹, L₁, L₂, L₃, and L₄ are as defined herein.

In certain embodiments, the CB₁ ligand comprises the structure

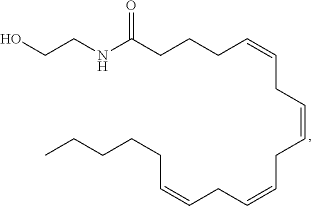 [SMILES: CCCCC/C=C\C/C=C\C/C=C\C/C=C\CCCC(=O)NCCO]

or a derivative thereof.

In some embodiments, the present disclosure provides compounds, and stereoisomers, tautomers, prodrugs, and salts thereof, comprising the structure of Formula (III):

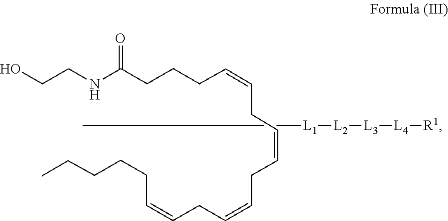

wherein R¹, L₁, L₂, L₃, and L₄ are as defined herein.

In some embodiments, the present disclosure provides compounds, and stereoisomers, tautomers, prodrugs, and salts thereof, comprising the structure of Formula (III-a):

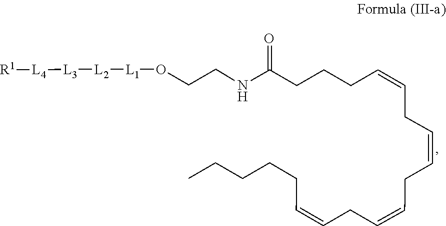

wherein R¹, L₁, L₂, L₃, and L₄ are as defined herein.

In some aspects, the present disclosure provides compounds, and stereoisomers, tautomers, prodrugs, and salts thereof, comprising the structure of Formula (VIII):

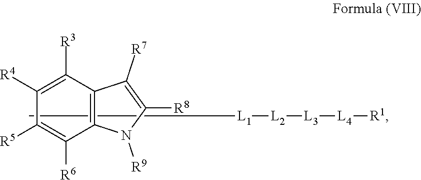

wherein:
each of L₁, L₂, L₃, and L₄ is independently a linker, a bond, or absent;
R¹ comprises one or more oligonucleotides, protecting groups, small molecules, proteins, antibodies, and/or peptides;
R³, R⁴, R⁵, R⁶, and R⁸ are each independently hydrogen, halogen, optionally substituted alkyl, optionally substituted heteroalkyl, optionally substituted cycloalkyl, optionally substituted heterocycloalkyl, optionally substituted aryl, or optionally substituted heteroaryl;
R⁹ is hydrogen, optionally substituted alkyl, or optionally substituted heteroalkyl; or R⁶ and R⁹ substituents may be joined together form an optionally substituted heterocycloalkyl or optionally substituted heteroaryl;
R⁷ is hydrogen, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D};
—NR^{7B}C(O)OR^{7D}, —NR^{7B}OR^{7D}, optionally substituted alkyl, optionally substituted heteroalkyl, optionally substituted cycloalkyl, optionally substituted heterocycloalkyl, optionally substituted aryl, or optionally substituted heteroaryl;
R^{7A}, R^{7B}, R^{7C}, R^{7D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{7B} and R^{7C} substituents bonded to the same nitrogen atom may optionally be joined to form a substituted or unsubstituted heterocycloalkyl or substituted or unsubstituted heteroaryl;
n7 is 0, 1, 2, 3, or 4; and
v7 is 1 or 2.

In some embodiments, R⁷ and R⁸ are each independently hydrogen, optionally substituted alkyl, or optionally substituted heteroalkyl. In some embodiments, R⁴ is halogen; and R³, R⁵, and R⁶ are each independently hydrogen. In some embodiments, R³, R⁴, R⁵, and R⁶ are each independently hydrogen. In certain embodiments, R⁶ and R⁹ substituents are joined together to form an optionally substituted heterocycloalkyl or optionally substituted heteroaryl. In some embodiments, R⁹ is hydrogen or optionally substituted alkyl.

In some embodiments, the present disclosure provides compounds, and stereoisomers, tautomers, prodrugs, and salts thereof, comprising the structure of Formula (VIII-a):

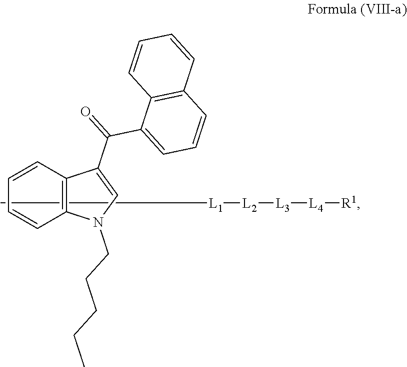

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-a-1):

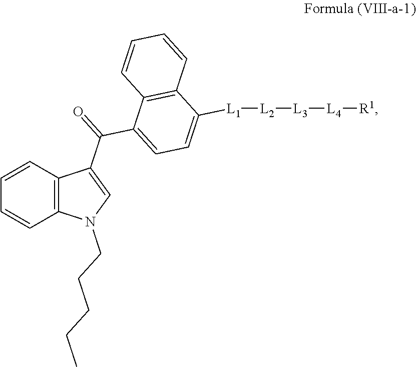

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-a-2):

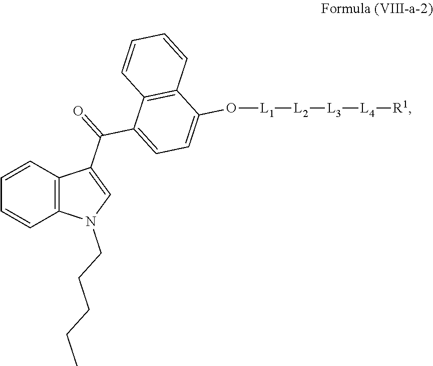

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-b):

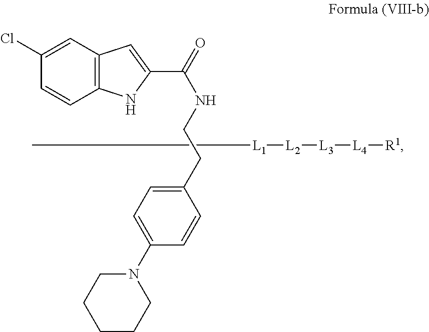

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-b-1):

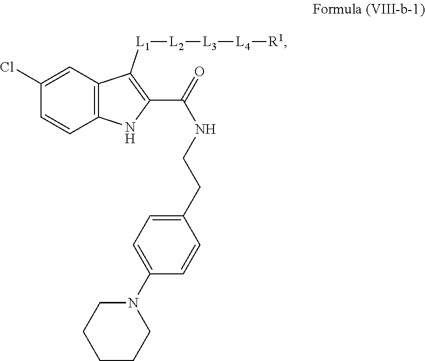

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-c):

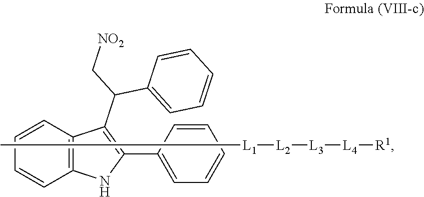

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-c-1):

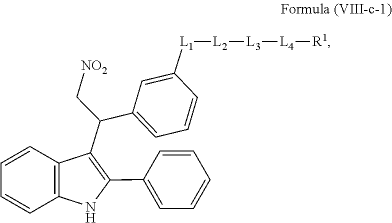

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-c-2):

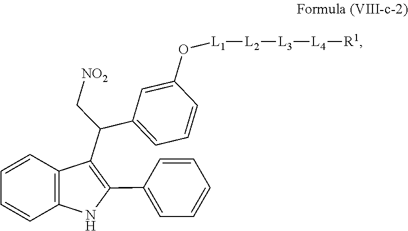

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-d):

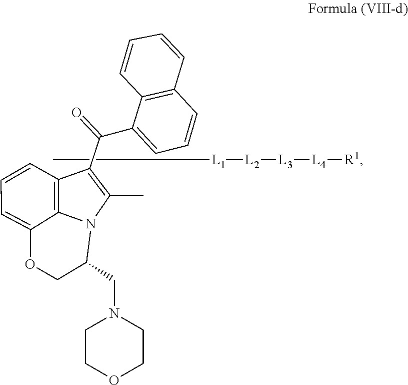

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-d-1):

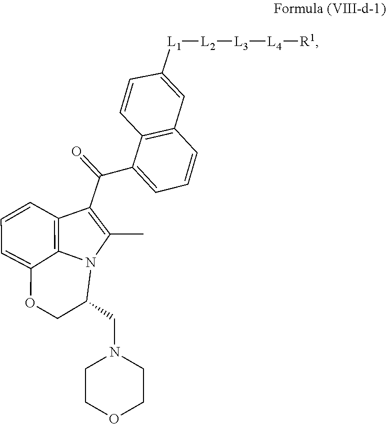

wherein R¹, L₁, L₂, L₃, and L₄ are as defined herein.

In some embodiments, the present disclosure provides compounds, and stereoisomers, tautomers, prodrugs, and salts thereof, comprising the structure of Formula (VIII-d-2):

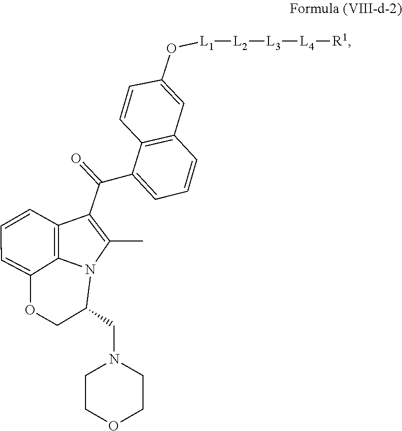

wherein R¹, L₁, L₂, L₃, and L₄ are as defined herein.

Tn some embodiments, the present disclosure provides compounds, and stereoisomers, tautomers, prodrugs, and salts thereof, comprising the structure of Formula (IX):

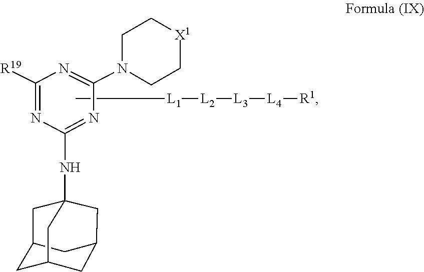

wherein:
each of L₁, L₂, L₃, and L₄ is independently a linker, a bond, or absent;
R¹ comprises one or more oligonucleotides, protecting groups, small molecules, proteins, antibodies, and/or peptides;
X¹ is NR¹⁰ or CR¹¹R¹²;
R¹⁰, R¹¹, and R¹² are each independently hydrogen, optionally substituted alkyl, optionally substituted heteroalkyl, optionally substituted cycloalkyl, optionally substituted heterocycloalkyl, optionally substituted aryl, or optionally substituted heteroaryl; and
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D};
—NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C} substituents bonded to the same nitrogen atom may optionally be joined to form a substituted or unsubstituted heterocycloalkyl or substituted or unsubstituted heteroaryl;
n19 is 0, 1, 2, 3, or 4; and
v19 is 1 or 2.

In some embodiments, the present disclosure provides compounds, and stereoisomers, tautomers, prodrugs, and salts thereof, comprising the structure of Formula (IX-a-1):

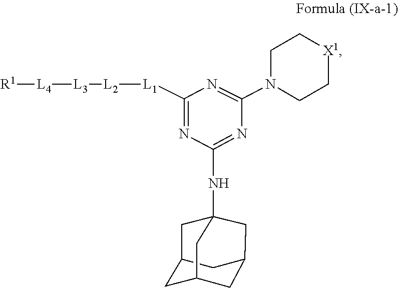

wherein X¹, R¹, L₁, L₂, L₃, and L₄ are as defined herein.

In some embodiments, the present disclosure provides compounds, and stereoisomers, tautomers, prodrugs, and salts thereof, comprising the structure of Formula (IX-a-2):

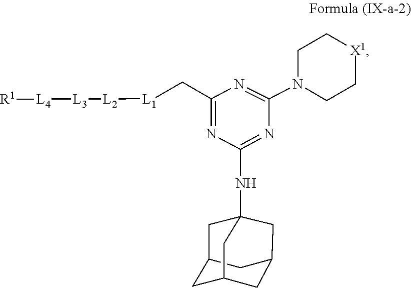

wherein X¹, R¹, L₁, L₂, L₃, and L₄ are as defined herein.

In some embodiments, X¹ is NR¹⁰; and R¹⁰ is hydrogen or optionally substituted alkyl. In some embodiments, R¹⁰ is hydrogen, —CH₃, or —CH₂CH₂F. In some embodiments, X¹ is CR¹¹R¹²; and R¹¹ and R¹² are each independently hydrogen or optionally substituted alkyl. In some embodiments, R¹¹ is hydrogen, —CH₃, or —CH₂CH₂F; and R¹² is hydrogen.

In some embodiments, the present disclosure provides compounds, and stereoisomers, tautomers, prodrugs, and salts thereof, comprising the structure of Formula (X):

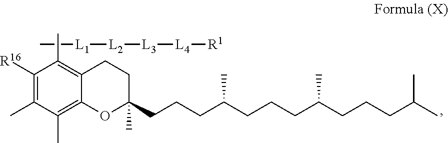

wherein:
each of L₁, L₂, L₃, and L₄ is independently a linker, a bond, or absent;
R¹ comprises one or more oligonucleotides, protecting groups, small molecules, proteins, antibodies, and/or peptides;
R¹⁶ is hydrogen, halogen, —CN, —N₃, —NO₂, —NR^{16B}R^{16C}, —C(O)R^{16D}, —C(O)OR^{16D}, —C(O)NR^{16B}R^{16C}, —OR^{16A}, —NR^{16B}C(O)R^{16D}, optionally substituted alkyl, optionally substituted heteroalkyl, optionally substituted cycloalkyl, optionally substituted heterocycloalkyl, optionally substituted aryl, or optionally substituted heteroaryl; and
R^{16A}, R^{16B}, R^{16C}, and R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{16B} and R^{16C} substituents bonded to the same nitrogen atom may optionally be joined to form a substituted or unsubstituted heterocycloalkyl or substituted or unsubstituted heteroaryl.

In some embodiments, the present disclosure provides compounds, and stereoisomers, tautomers, prodrugs, and salts thereof, comprising the structure of Formula (X-a):

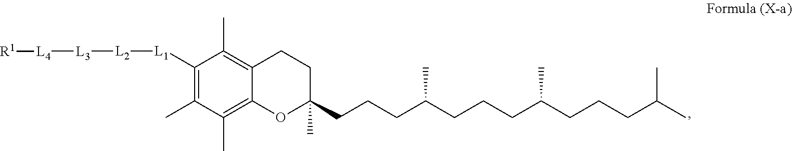

wherein R¹, L₁, L₂, L₃, and L₄ are as defined herein.

In some embodiments, the present disclosure provides compounds, and stereoisomers, tautomers, prodrugs, and salts thereof, comprising the structure of Formula (X-a-1):

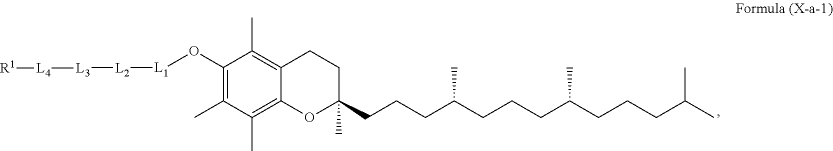

wherein R¹, L₁, L₂, L₃, and L₄ are as defined herein.

In certain embodiments, the CB₁ ligand comprises the structure:

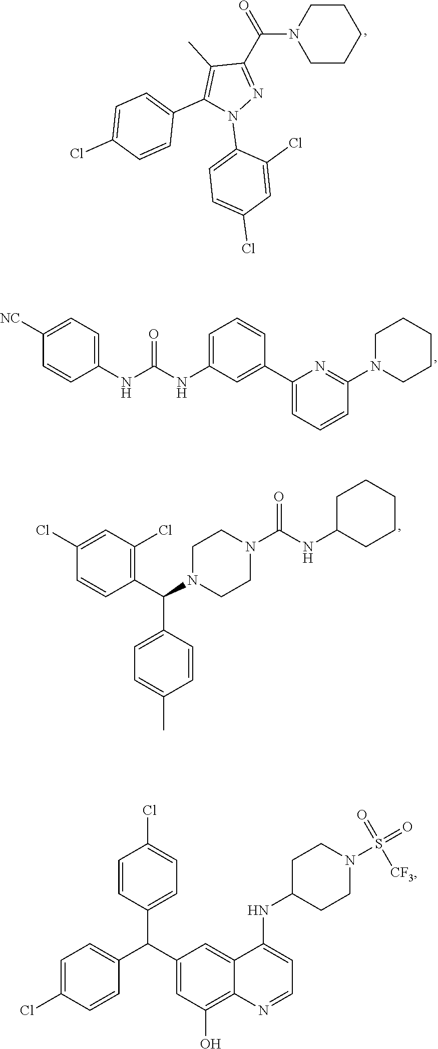 [SMILES: Cc1c(C(=O)N2CCCCC2)nn(-c2ccc(Cl)cc2Cl)c1-c1ccc(Cl)cc1 ; N#Cc1ccc(NC(=O)Nc2cccc(-c3cccc(N4CCCCC4)n3)c2)cc1 ; Cc1ccc([C@@H](c2ccc(Cl)cc2Cl)N2CCN(C(=O)NC3CCCCC3)CC2)cc1 ; O=S(=O)(N1CCC(Nc2ccnc3c(O)cc(C(c4ccc(Cl)cc4)c4ccc(Cl)cc4)cc23)CC1)C(F)(F)F]

or a derivative thereof.

In some embodiments, the compound comprises the structure:

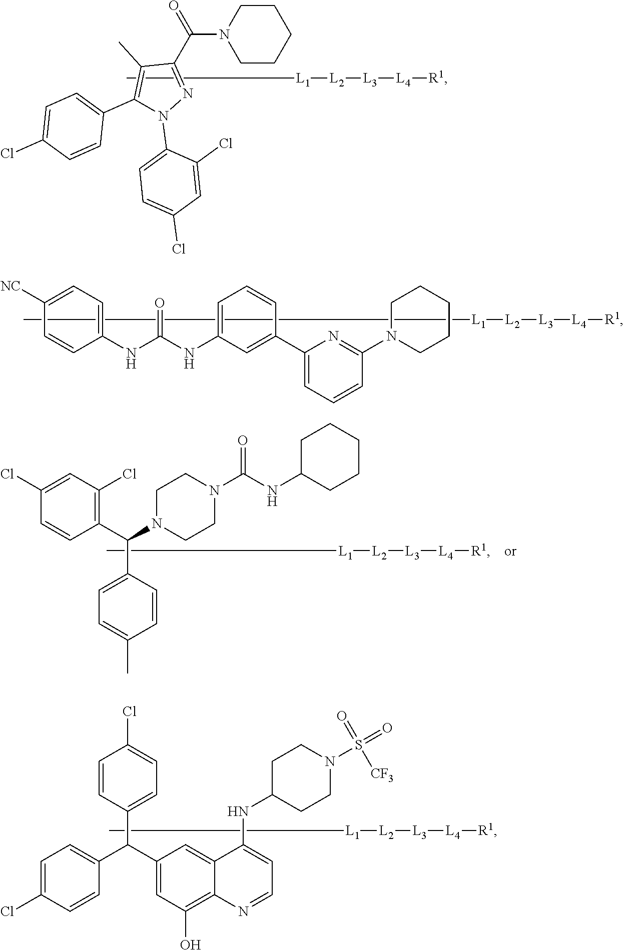

wherein R¹, L₁, L₂, L₃, and L₄ are as defined herein.

In some embodiments, each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, oxygen, optionally substituted nitrogen, an amide, a phosphodiester bond, or a phosphorothioate bond.

In certain embodiments, L₁ is a bond. In certain embodiments, L₁ is oxygen. In certain embodiments, L₁ comprises the structure

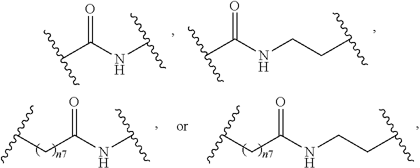

wherein n7 is 0, 1, 2, 3, 4, 5, 6, 7, 8, 9, or 10.

In some embodiments, L₂ is an optionally substituted PEG linker. In some embodiments, the PEG linker is 1, 2, 3, 4, 5, 6, 7, 8, 9, or 10 PEG units in length. In some embodiments, L₂ is an optionally substituted alkyl linker. In certain embodiments, L₂ comprises the structure

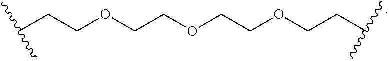 [SMILES: *CCOCCOCCOCC*]

In some embodiments, L₄ is an optionally substituted heteroalkyl linker or a bond. In certain embodiments, the heteroalkyl linker is substituted with one or more ═O substituents. In some embodiments, L₄ comprises the structure

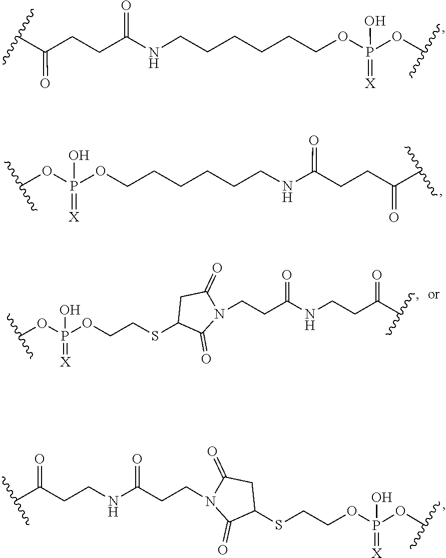

wherein X is O or S. In certain embodiments, L₄ comprises the structure

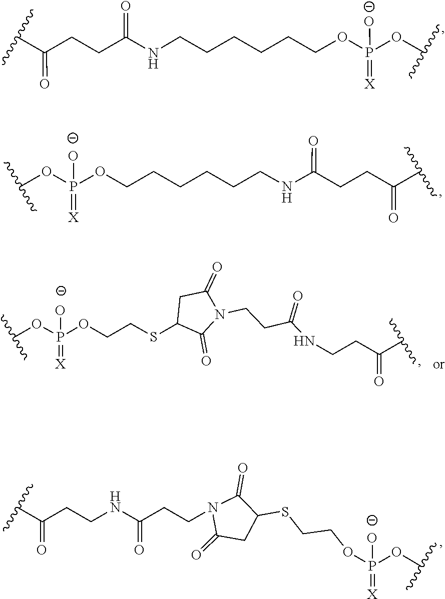

wherein X is O or S.

In some embodiments, L₃ is an optionally substituted heteroaryl linker. In some embodiments, one of L₃ and L₄ is an optionally substituted phosphodiester bond or an optionally substituted phosphorothioate bond, and the other of L₃ and L₄ is a bond. In some embodiments, L₃ is an optionally substituted partially unsaturated heteroaryl or optionally substituted partially unsaturated heterocycloalkyl linker. In certain embodiments, L₃ comprises the structure

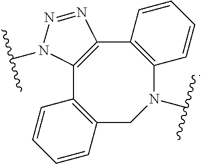 [SMILES: *N1Cc2ccccc2-c2c(nnn2*)-c2ccccc21]

In some embodiments, L₁, L₂, L₃, and L₄ together comprise the structure:

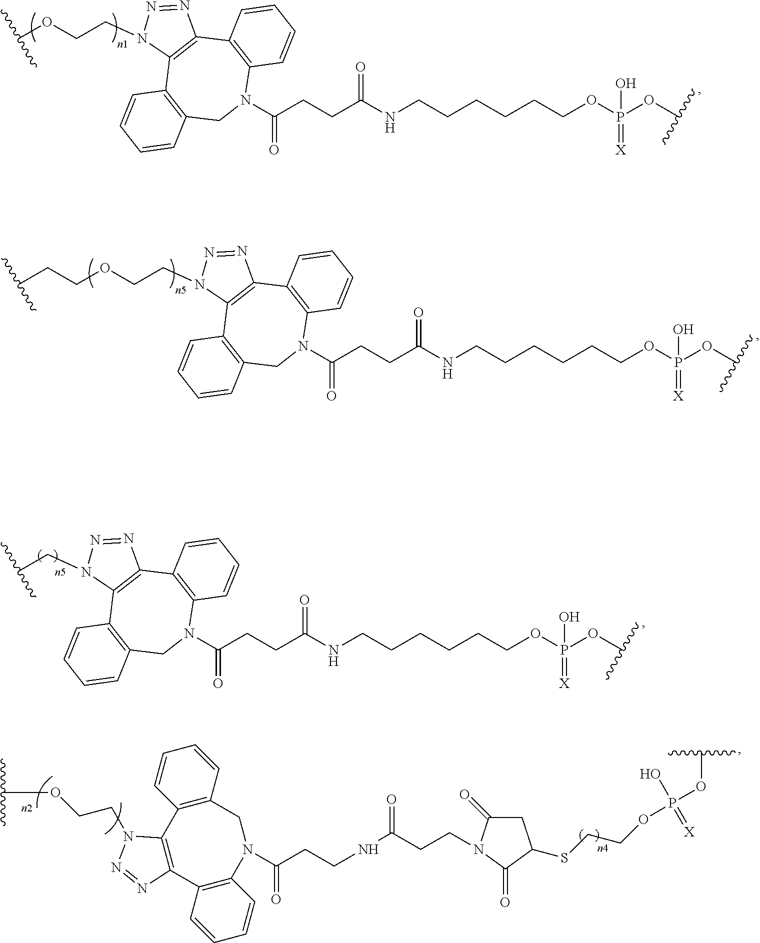

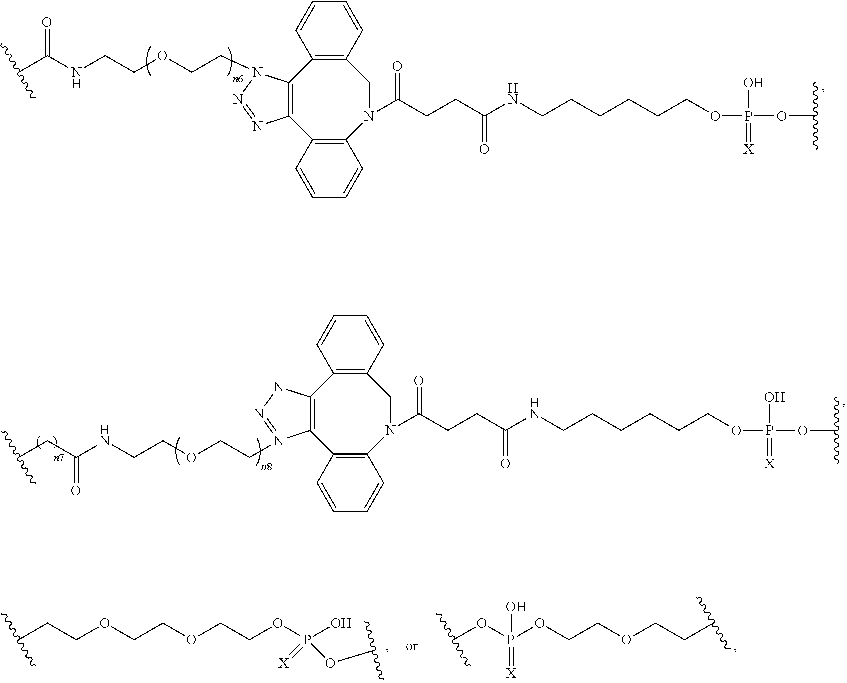

wherein:
X is O or S;
n1, n2, n4, n5, n6, n7, and n8 are each independently 0, 1, 2, 3, 4, 5, 6, 7, 8, 9, or 10; and
n3 is 0, 1, 2, 3, 4, 5, 6, 7, 8, 9, 10, 11, 12, 13, 14, 15, 16, 17, 18, 19, 20, 21, or 22.

In certain embodiments, L₁, L₂, L₃, and L₄ together comprise the structure:

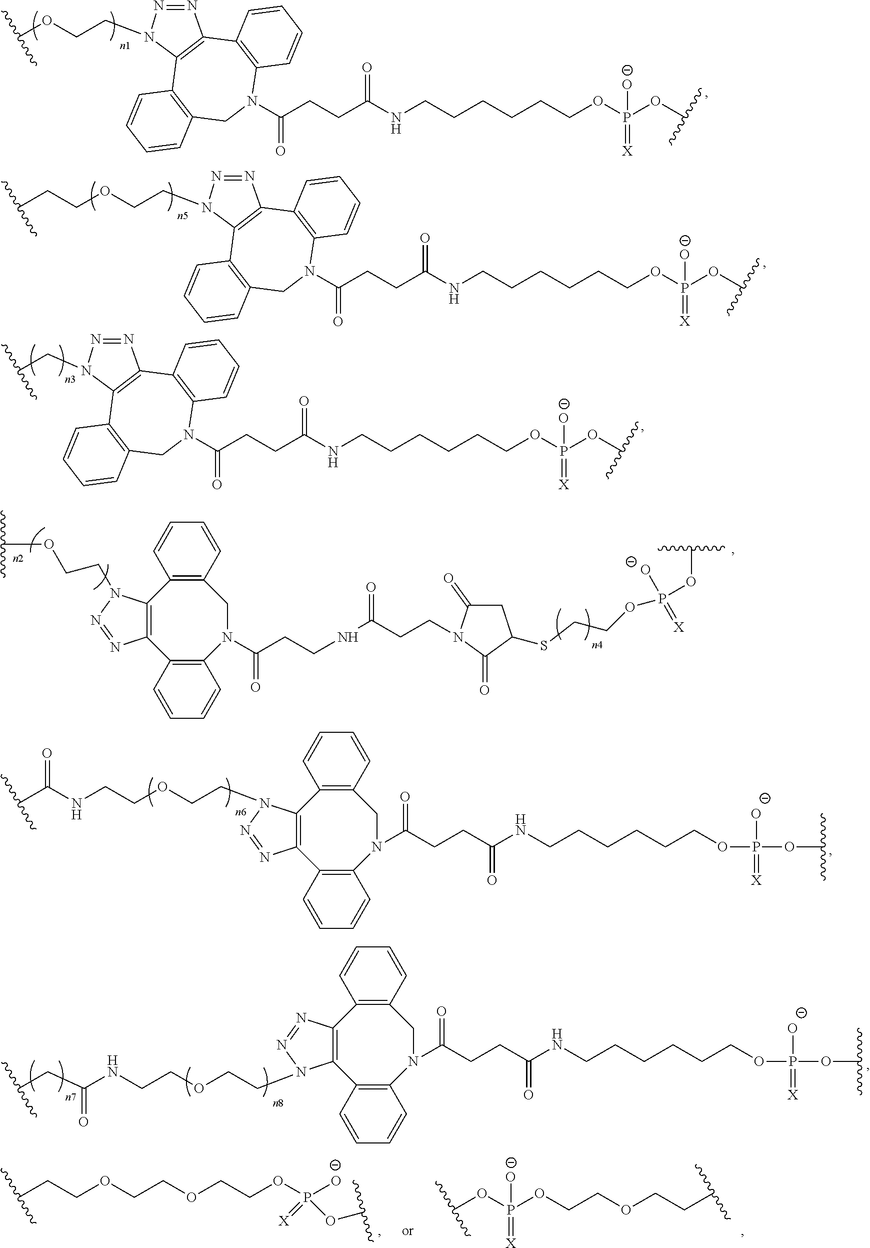

wherein:
X is O or S; and
n1, n2, n4, n5, n6, n7, and n8 are each independently 0, 1, 2, 3, 4, 5, 6, 7, 8, 9, or 10; and
n3 is 0, 1, 2, 3, 4, 5, 6, 7, 8, 9, 10, 11, 12, 13, 14, 15, 16, 17, 18, 19, 20, 21, or 22.

In some embodiments, X is S. In some embodiments, X is O.

In another aspect, the present disclosure provides compounds, and stereoisomers, tautomers, prodrugs, and salts thereof, comprising the structure of Formula (IV′), or a salt thereof:

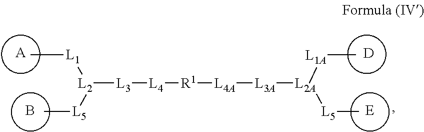

wherein:

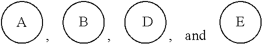

and are each independently a cannabinoid receptor type 1 (CB₁) ligand, or one of

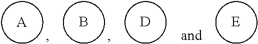

is a CB₁ ligand, and the other of

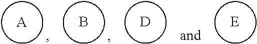

comprises a lipid or a ligand;
each of L₁, L₂, L₃, L₄, L₅, L_{1A}, L_{2A}, L_{A3}, L_{A4}, and L_{A5} are each independently a linker, a bond, or absent; and
R¹ comprises one or more oligonucleotides, protecting groups, small molecules, proteins, antibodies, and/or peptides.

In some embodiments, the present disclosure provides compounds, and stereoisomers, tautomers, prodrugs, and salts thereof, comprising the structure of Formula (IV″):

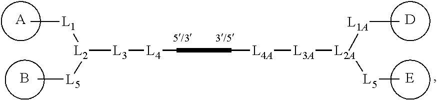

wherein:
[symbol] is an oligonucleotide, and

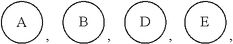

L₁, L₂, L₃, L₄, L₅, L_{1A}, L_{2A}, L_{A3}, L_{A4}, and L_{A5} are as defined herein.

In some embodiments, the present disclosure provides compounds, and stereoisomers, tautomers, prodrugs, and salts thereof, comprising the structure of Formula (IV″-a):

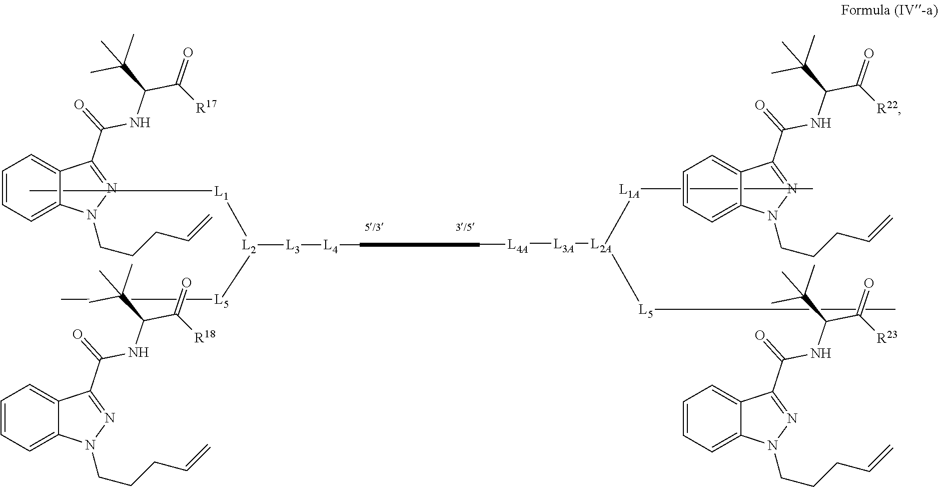

wherein R⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D};
—NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R^{18C}, —NHNR^{18B}R^{18C}, —ONR^{18B}R^{18C}, —NHC(O)NHNR^{18B}R^{18C}, —NHC(O)NR^{18B}R^{18C}, —NR^{18B}R^{18C}, —C(O)R^{18D}, —C(O)OR^{18D}, —C(O)NR^{18B}R^{18C}, —OR^{18A}, —NR^{18B}SO₂R^{18A}, —NR^{18B}C(O)R^{18D}, —NR^{18B}C(O)OR^{18D}, —NR^{18B}OR^{18D}, optionally substituted alkyl, optionally substituted heteroalkyl, optionally substituted cycloalkyl, optionally substituted heterocycloalkyl, optionally substituted aryl, or optionally substituted heteroaryl;
R²² is hydrogen, —SOₙ₂₂R^{22A}, —SOᵥ₂₂NR^{22B}R^{22C}, —NHNR^{22B}R^{22C}, —ONR^{22B}R^{22C}, —NHC(O)NHNR^{22B}R^{22C}, —NHC(O)NR^{22B}R^{22C}, —NR^{22B}R^{22C}, —C(O)R^{22D}, —C(O)OR^{22D}, —C(O)NR^{22B}R^{22C}, —OR^{22A}, —NR^{22B}SO₂R^{22A}, —NR^{22B}C(O)R^{22D};
—NR^{22B}C(O)OR^{22D}, —NR^{22B}OR^{22D}, optionally substituted alkyl, optionally substituted heteroalkyl, optionally substituted cycloalkyl, optionally substituted heterocycloalkyl, optionally substituted aryl, or optionally substituted heteroaryl;
R²³ is hydrogen, —SOₙ₂₃R^{23A}, —SOᵥ₂₃NR^{23B}R^{23C}, —NHNR^{23B}R^{23C}, ONR^{23B}R^{23C}, —NHC(O)NHNR^{23B}R^{23C}, —NHC(O)NR^{23B}R^{23C}, —NR^{23B}R^{23C}, —C(O)R^{23D}, —C(O)OR^{23D}, —C(O)NR^{23B}R^{23C}, —OR^{23A}, —NR^{23B}SO₂R^{23A}, —NR^{23B}C(O)R^{23D};
—NR^{23B}C(O)OR^{23D}, —NR^{23B}OR^{23D}, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, —SOₙ₂₄R^{24A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —NR^{24B}R^{24C}, —C(O)R^{24D}, —C(O)OR^{24D}, —C(O)NR^{24B}R^{24C}, —OR^{24A}, —NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, R^{17D}, R^{18A}, R^{18B}, R^{18C}, R^{18D}, R^{22A}, R^{22B}, R^{22C}, R^{22D}, R^{23A}, R^{23B}, R^{23C}, and R^{23D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl;
n17, n18, n22, and n23 are each independently 0, 1, 2, 3, or 4; and
v17, v18, v22, and v23 are each independently 1 or 2.

In some embodiments, the present disclosure provides compounds, and stereoisomers, tautomers, prodrugs, and salts thereof, comprising the structure of Formula (IV″-b):

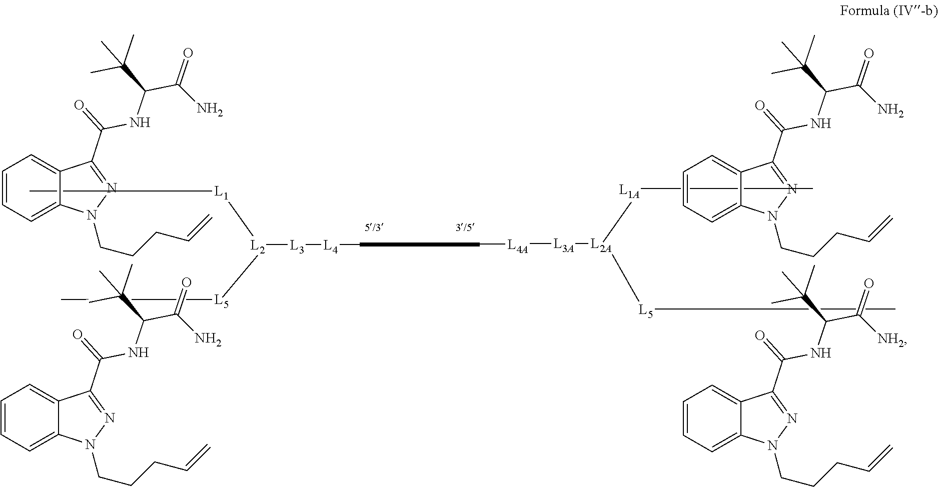

wherein L₁, L₂, L₃, L₄, L₅, L_{1A}, L_{2A}, L_{A3}, L_{A4}, and L_{A5} are as defined herein.

In some embodiments, the present disclosure provides compounds, and stereoisomers, tautomers, prodrugs, and salts thereof, comprising the structure of Formula (IV″-b-1):

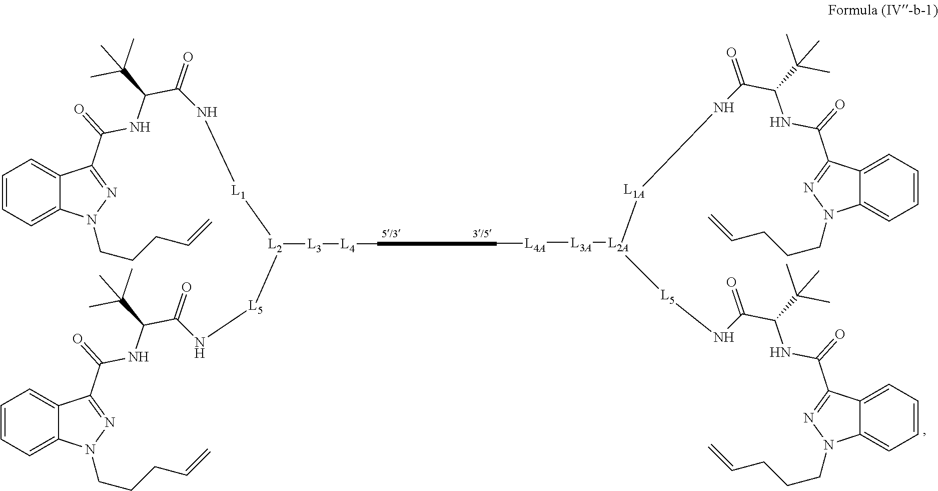

wherein L₁, L₂, L₃, L₄, L₅, L_{1A}, L_{2A}, L_{A3}, L_{A4}, and L_{A5} are as defined herein.

In some aspects, the present disclosure provides compounds, and stereoisomers, tautomers, prodrugs, and salts thereof, comprising the structure of Formula (IV), or a salt thereof:

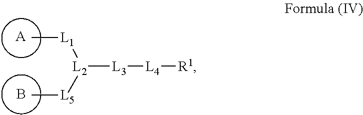

wherein:

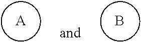

are each independently a cannabinoid receptor type 1 (CB₁) ligand, or one of

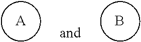

is a CB₁ ligand, and the other of

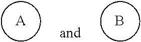

comprises a lipid or a ligand;
each of L₁, L₂, L₃, L₄, and L₅ is independently a linker, a bond, or absent; and
R¹ comprises one or more oligonucleotides, protecting groups, small molecules, proteins, antibodies, and/or peptides.

In some embodiments, wherein

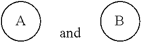

are each independently a CB₁ ligand. In some embodiments, one or both of the CB₁ ligands is a CB₁ agonist. In some embodiments, one or both of the CB₁ ligands is a CB₁ antagonist. In some embodiments, the CB₁ ligand is a selective ligand. In some embodiments, the CB₁ ligand is a non-selective ligand. In certain embodiments, each of the CB₁ ligands is independently selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an ani-CB₁ antibody, and derivatives thereof. In some embodiments, each of the CB₁ ligands comprises the structure

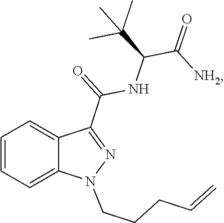 [SMILES: C=CCCCn1nc(C(=O)N[C@H](C(N)=O)C(C)(C)C)c2ccccc21]

or a derivative thereof.

In some embodiments, the present disclosure provides compounds, and stereoisomers, tautomers, prodrugs, and salts thereof, comprising the structure of Formula (V):

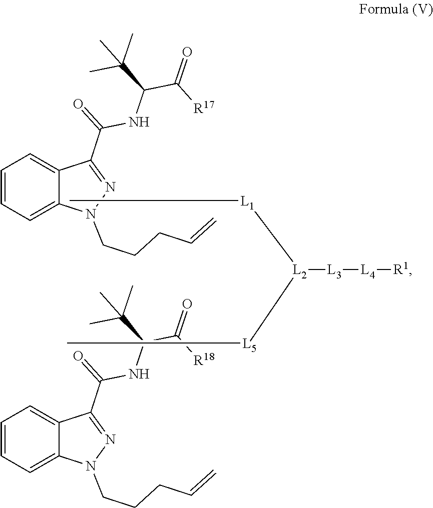

wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R^{18C}, —NHNR^{18B}R^{18C}, —ONR^{18B}R^{18C}, —NHC(O)NHNR^{18B}R^{18C}, —NHC(O)NR^{18B}R^{18C}, —NR^{18B}R^{18C}, —C(O)R^{18D}, —C(O)OR^{18D}, —C(O)NR^{18B}R^{18C}, —OR^{18A}, —NR^{18B}SO₂R^{18A}, —NR^{18B}C(O)R^{18D}, —NR^{18B}C(O)OR^{18D}, —NR^{18B}R^{18D} optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, R^{17B}, R^{18A}, R^{18B}, R^{18C}, and R^{18D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl; R^{17B} and R^{17C} or R^{18B} and R^{18C} substituents bonded to the same nitrogen atom may optionally be joined to form a substituted or unsubstituted heterocycloalkyl or substituted or unsubstituted heteroaryl;
n17 and n18 are each independently 0, 1, 2, 3, or 4; and
v17 and v18 are each independently 1 or 2.

In some embodiments, the present disclosure provides compounds, and stereoisomers, tautomers, prodrugs, and salts thereof, comprising the structure of Formula (V-a):

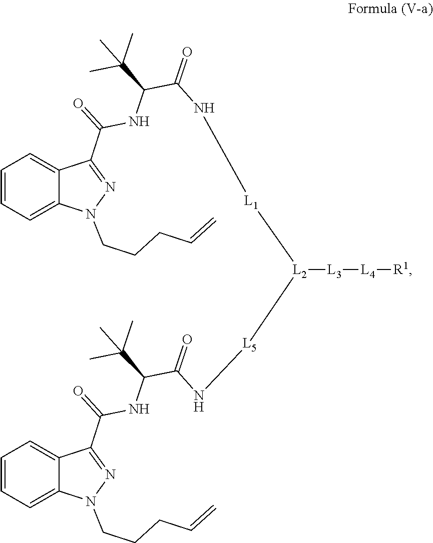

wherein R¹, L₁, L₂, L₃, L₄, and L₅ are as defined herein.

In some embodiments, the present disclosure provides compounds, and stereoisomers, tautomers, prodrugs, and salts thereof, comprising the structure:

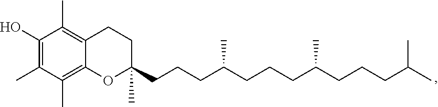 [SMILES: Cc1c(C)c2c(c(C)c1O)CC[C@@](C)(CCC[C@H](C)CCC[C@H](C)CCCC(C)C)O2]

or a derivative thereof, and the other CB₁ ligand comprises the structure:

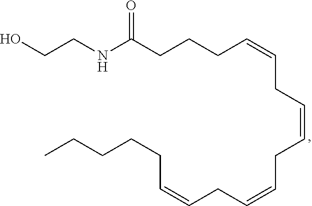 [SMILES: CCCCC/C=C\C/C=C\C/C=C\C/C=C\CCCC(=O)NCCO]

or a derivative thereof.

In some embodiments, the present disclosure provides compounds, and stereoisomers, tautomers, prodrugs, and salts thereof, comprising the structure of Formula (IX):

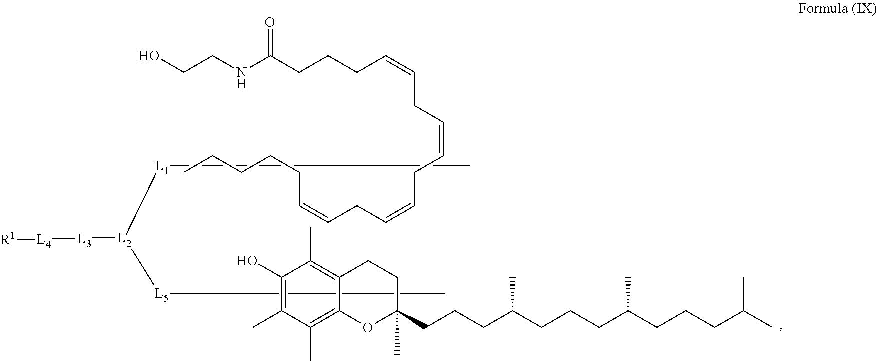

wherein R¹, L₁, L₂, L₃, L₄, and L₅ are as defined herein.

In some embodiments, the present disclosure provides compounds, and stereoisomers, tautomers, prodrugs, and salts thereof, comprising the structure of Formula (IX-a):

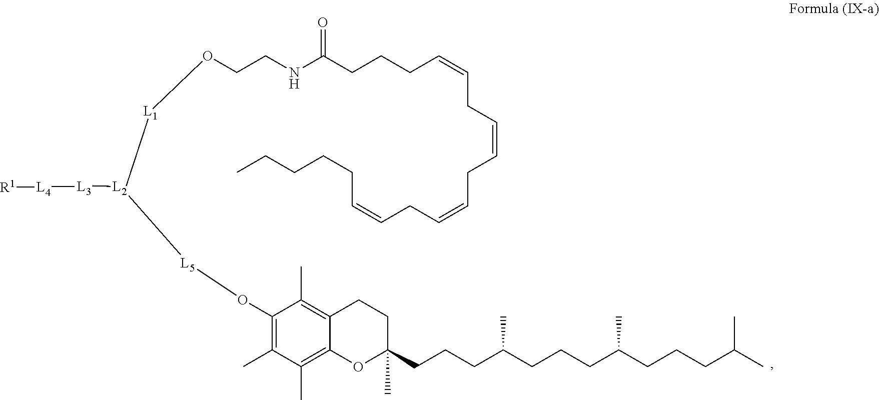

wherein R¹, L₁, L₂, L₃, L₄, and L₅ are as defined herein.

In some embodiments, the present disclosure provides compounds, and stereoisomers, tautomers, prodrugs, and salts thereof, wherein each of the CB₁ ligands independently comprises the structure:

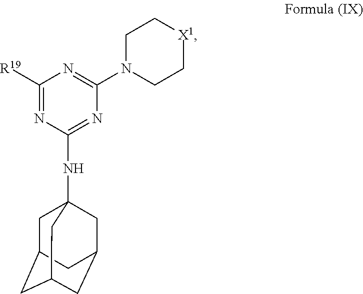

wherein:
X¹ is NR¹⁰ or CR¹¹R¹²
R¹⁰, R¹¹, and R¹² are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D}, —NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, and R^{19D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C} substituents bonded to the same nitrogen atom may optionally be joined to form a substituted or unsubstituted heterocycloalkyl or substituted or unsubstituted heteroaryl;
n19 is 0, 1, 2, 3, or 4; and
v19 is 1 or 2.

In some embodiments, the present disclosure provides compounds, and stereoisomers, tautomers, prodrugs, and salts thereof, comprising the structure of Formula (XI):

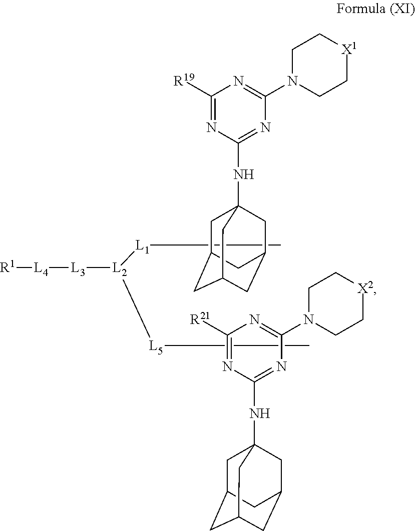

wherein:
L₁, L₂, L₃, L₄, and L₅ are as defined herein;
X¹ is NR¹⁰ or CR¹¹R¹²;
X² is NR¹³ or CR¹⁴R¹⁵;
R¹⁰, R¹¹, R¹², R¹³, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D}, R^{21A}, R^{21B}, R^{21C}, and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{21B} and R^{21C} substituents bonded to the same nitrogen atom may optionally be joined to form a substituted or unsubstituted heterocycloalkyl or substituted or unsubstituted heteroaryl;
n19 and n21 are each independently 0, 1, 2, 3, or 4; and
v19 and v21 are each independently 1 or 2.

In some embodiments, the present disclosure provides compounds, and stereoisomers, tautomers, prodrugs, and salts thereof, comprising the structure of Formula (XI-a):

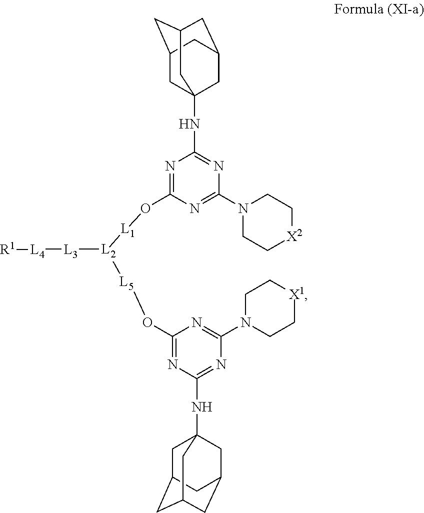

wherein X¹, X², L₁, L₂, L₃, L₄, and L₅ are as defined herein.

In some embodiments, X¹ is NR¹⁰; X² is NR¹³; and R¹⁰ and R¹³ are each independently hydrogen or optionally substituted alkyl. In some embodiments, R¹⁰ and R¹³ are each independently hydrogen, —CH₃, or —CH₂CH₂F. In some embodiments, X¹ is CR¹¹R¹²; X² is CR¹⁴R¹⁵; and R¹¹, R¹, R¹⁴, and R¹⁵ are each independently hydrogen or optionally substituted alkyl. In some embodiments, R¹² and R¹⁵ are each independently hydrogen, —CH₃, or —CH₂CH₂F; and R¹¹ and R¹⁴ are each independently hydrogen.

In some embodiments, each of L₁, L₂, L₃, L₄, and L₅ is independently absent, a bond, an optionally substituted alkyl linker, an optionally substituted polyethylene glycol (PEG) linker, an optionally substituted heteroalkyl linker, an optionally substituted heteroaryl linker, oxygen, optionally substituted nitrogen, an amide, a phosphodiester bond, or a phosphorothioate bond.

In some embodiments, L₁ and L₅ are each an optionally substituted PEG linker. In some embodiments, L₁ and L₅ are each an optionally substituted PEG linker of 1, 2, 3, 4, 5, 6, 7, 8, 9, or 10 PEG units in length.

In some embodiments, L₂ is an optionally substituted heteroalkyl linker. In some embodiments, the heteroalkyl linker is substituted with one or more ═O substituents. In certain embodiments, L₂ comprises the structure

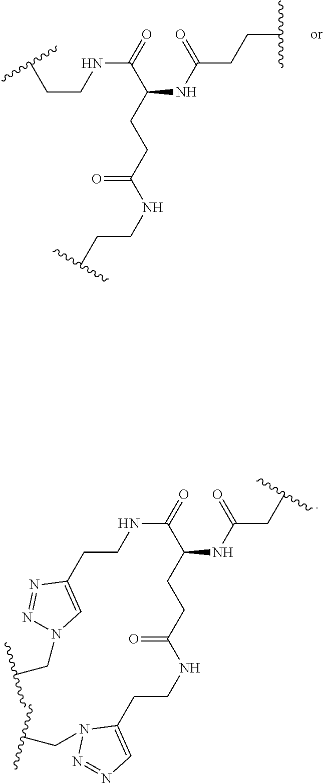 [SMILES: *CCNC(=O)CC[C@H](NC(=O)CC*)C(=O)NCC* ; *CC(=O)N[C@@H](CCC(=O)NCCc1cnnn1C*)C(=O)NCCc1cn(C*)nn1]

In some embodiments, L₃ is an optionally substituted heteroaryl linker. In some embodiments, L₃ is an optionally substituted partially unsaturated heteroaryl linker or optionally substituted partially unsaturated heterocycloalkyl linker. In certain embodiments, L₃ comprises the structure

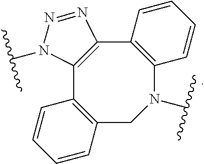 [SMILES: *N1Cc2ccccc2-c2c(nnn2*)-c2ccccc21]

In some embodiments, L₄ is an optionally substituted heteroalkyl linker. In some embodiments, heteroalkyl linker is substituted with one or more ═O substituents. In some embodiments, L₄ comprises the structure

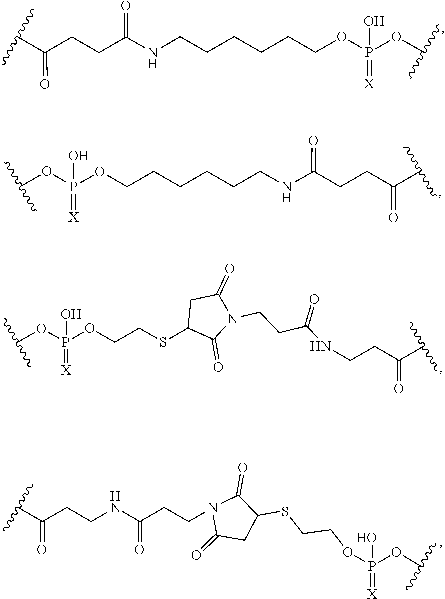

wherein X is O or S. In certain embodiments, L₄ comprises the structure

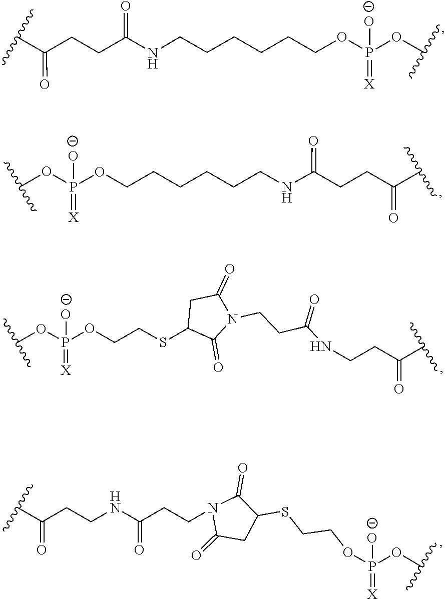

wherein X is O or S.

In some embodiments, X is S. In some embodiments, X is O.

In some embodiments, L₁, L₂, L₃, L₄, and L₅ together comprise the structure:

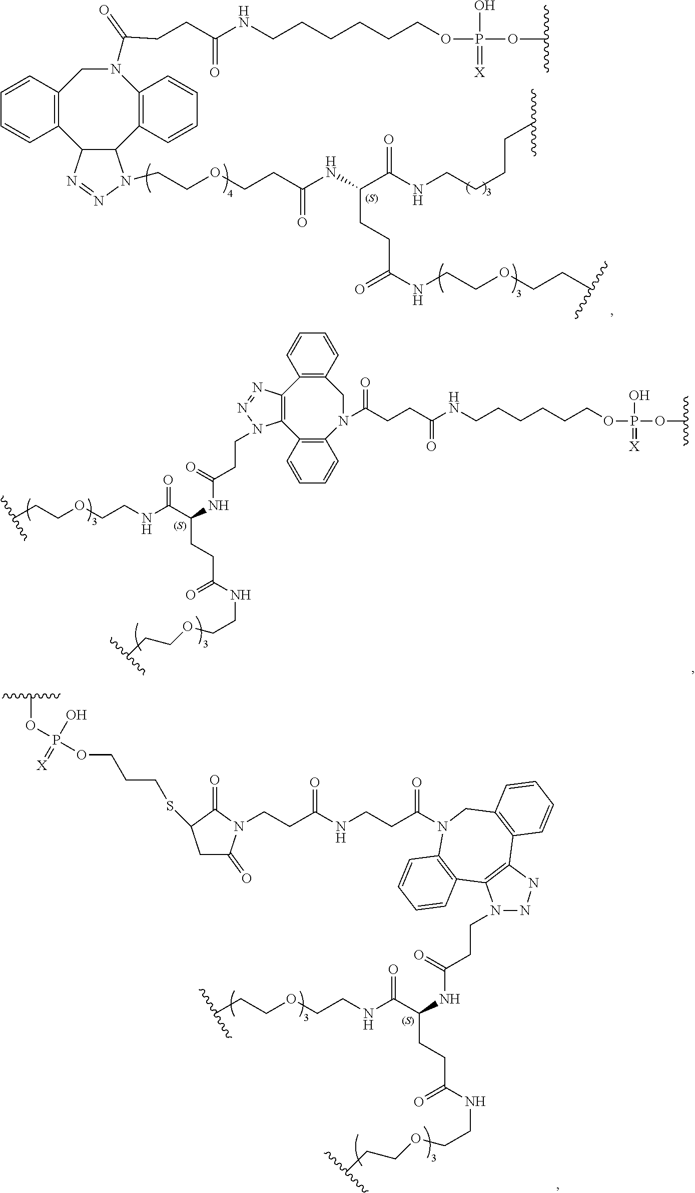

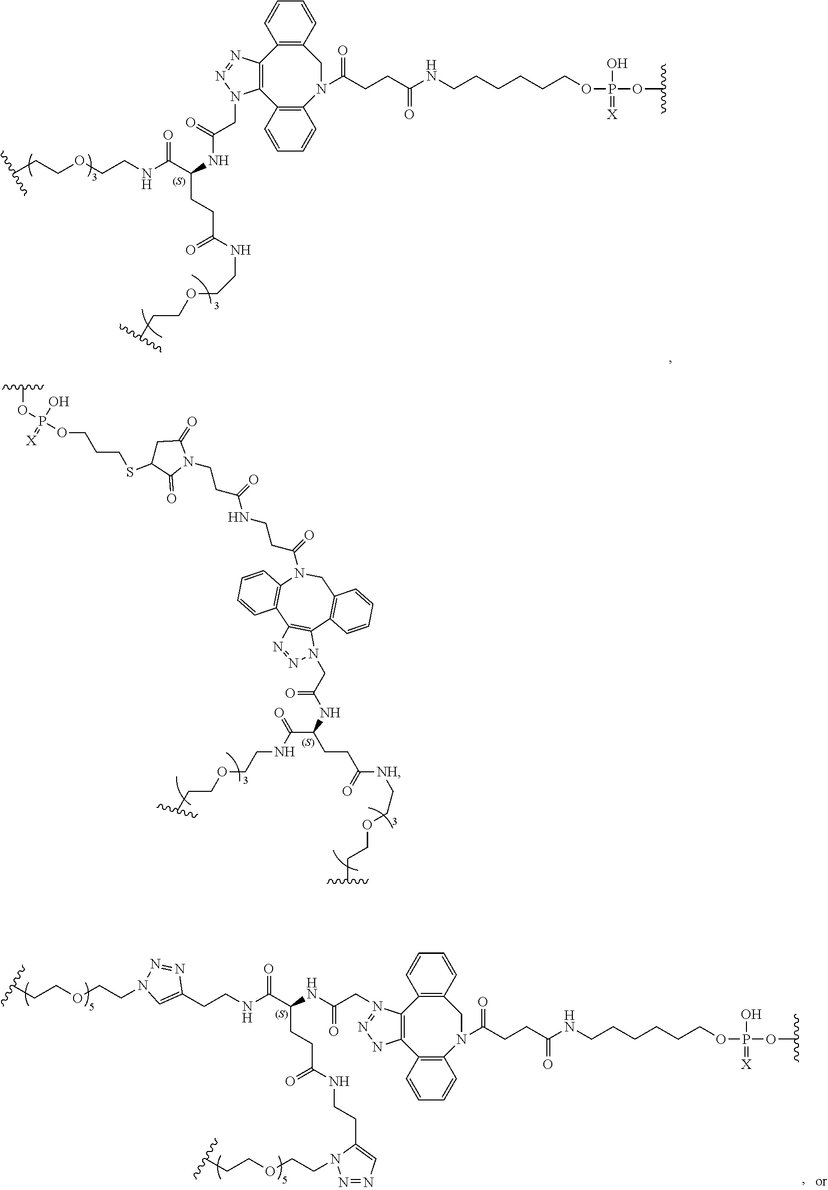

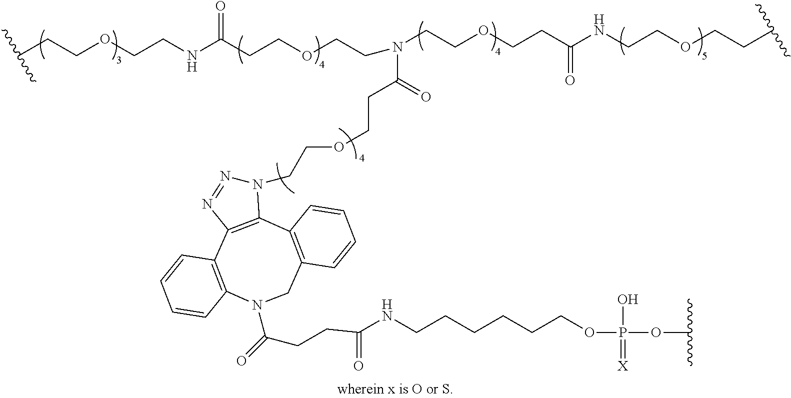

In certain embodiments, L₁, L₂. L₃, L₄, and L₅ together comprise the structure:

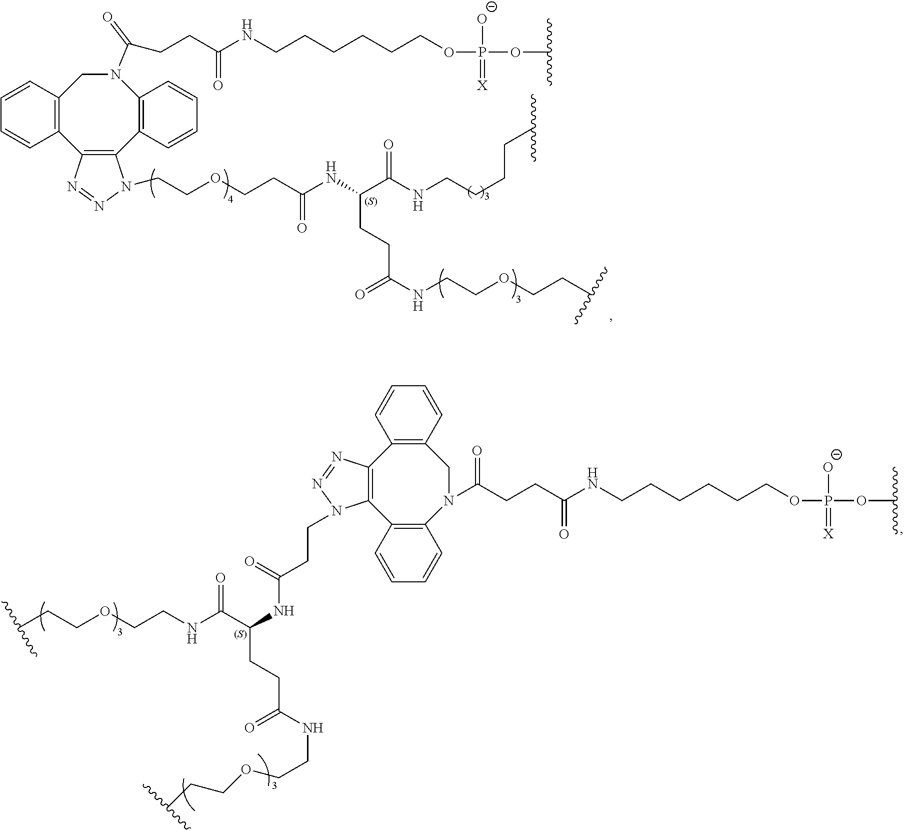

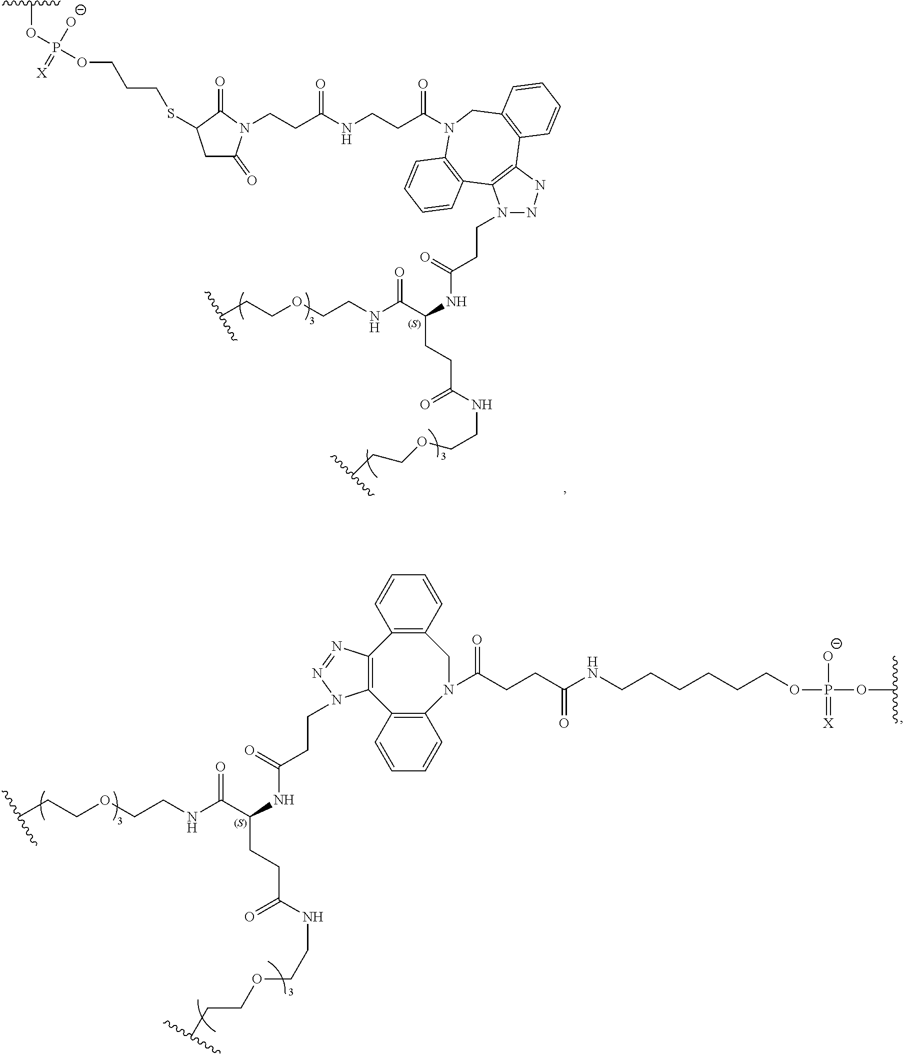

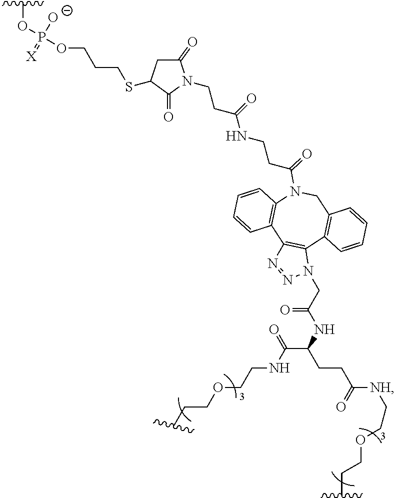

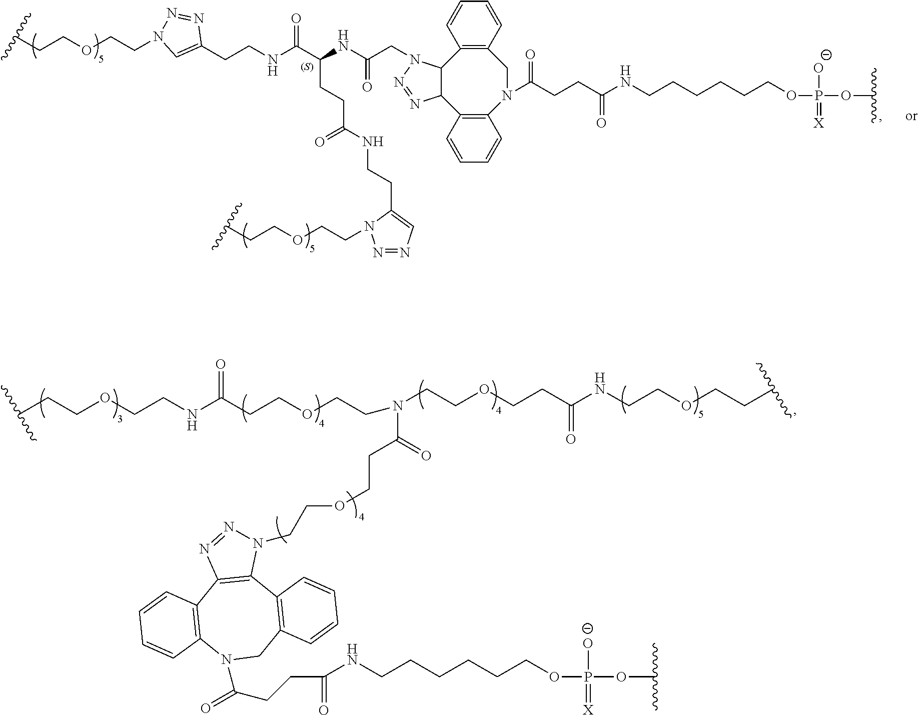

wherein X is O or S.

In some aspects, the present disclosure provides compounds, and stereoisomers, tautomers, prodrugs, and salts thereof, comprising the structure of Formula (VI), or a salt thereof:

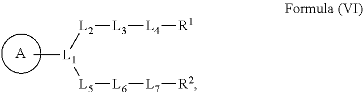

wherein:

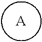

is a cannabinoid receptor type 1 (CB₁) ligand;
each of L₁, L₂, L, L₄, L₅, L₆, and L₇ is independently a linker, a bond, or absent; and
R¹ and R² each independently comprise one or more oligonucleotides, protecting groups, small molecules, proteins, antibodies, and/or peptides.

In some embodiments, the CB₁ ligand is a CB₁ agonist. In some embodiments, the CB₁ ligand is a CB₁ antagonist. In certain embodiments, the CB₁ ligand is selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof. In certain embodiments, the CB₁ ligand comprises the structure

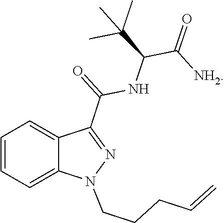 [SMILES: C=CCCCn1nc(C(=O)N[C@H](C(N)=O)C(C)(C)C)c2ccccc21]

In some embodiments, the present disclosure provides compounds, and stereoisomers, tautomers, prodrugs, and salts thereof, comprising the structure of Formula (VII):

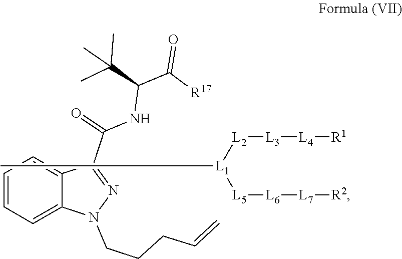

wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, and R^{17D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl;
n17 is 0, 1, 2, 3, or 4; and
v17 is 1 or 2.

In some embodiments, the present disclosure provides compounds, and stereoisomers, tautomers, prodrugs, and salts thereof, comprising the structure of Formula (VII-a):

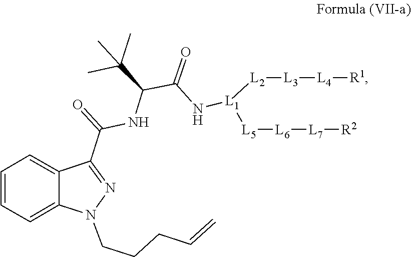

wherein R¹, R², L₁, L₂, L₃, L₄, L₅, L₆, and L₇ are as defined herein.

In some embodiments, each of L₁, L₂, L₃, L₄, L₅, L₆, and L₇ is independently absent, a bond, an optionally substituted alkyl linker, an optionally substituted polyethylene glycol (PEG) linker, an optionally substituted heteroalkyl linker, an optionally substituted heteroaryl linker, an optionally substituted saturated or partially unsaturated heterocycloalkyl linker, oxygen, optionally substituted nitrogen, an amide, a phosphodiester bond, or a phosphorothioate bond.

In some embodiments, L₁ is an optionally substituted PEG linker. In certain embodiments, L₁ is an optionally substituted PEG linker which is 1, 2, 3, 4, 5, 6, 7, 8, 9, or 10 PEG units in length.

In some embodiments, L₂ and L₅ are each independently an optionally substituted PEG linker. In some embodiments, L₂ and L₅ are each independently an optionally substituted PEG linker three or four PEG units in length. In certain embodiments, L₁, L₂, and L₅ together comprise the structure

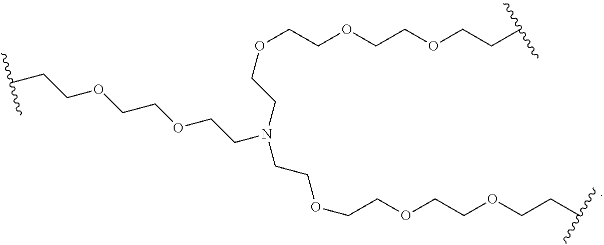 [SMILES: *CCOCCOCCOCCN(CCOCCOCC*)CCOCCOCCOCC*]

In some embodiments, L₃ and L₆ are each independently an optionally substituted heteroaryl linker or an optionally substituted heterocycloalkyl linker. In some embodiments, L₃ and L₆ are each independently an optionally substituted partially unsaturated heteroaryl linker or an optionally substituted partially unsaturated heterocycloalkyl linker. In certain embodiments, L₃ and L₆ each comprise the structure

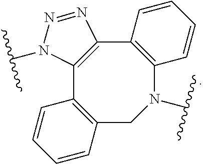 [SMILES: *N1Cc2ccccc2-c2c(nnn2*)-c2ccccc21]

In some embodiments, L₄ and L₇ are each independently an optionally substituted heteroalkyl linker. In some embodiments, the heteroalkyl linker is substituted with one or more ═O substituents. In some embodiments, L₄ and L₇ each comprise the structure

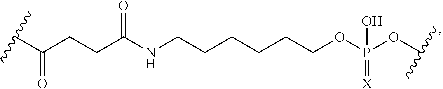

wherein X is O or S. In certain embodiments, L₄ and L₇ each comprise the structure

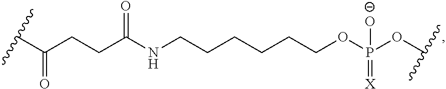

wherein X is O or S.

In some embodiments, R¹ comprises an oligonucleotide. In certain embodiments, the oligonucleotide is attached at its 5′ end. In certain embodiments, the oligonucleotide is attached at its 3′ end. In certain embodiments, the oligonucleotide is attached at an internal position on the oligonucleotide. In some embodiments, the internal position is an internucleoside linkage. In some embodiments, R¹ comprises an oligonucleotide conjugated to one or more additional CB₁ ligands. In certain embodiments, the oligonucleotide is conjugated to two, three, four, five, or more than five additional CB₁ ligands. In some embodiments, additional CB₁ ligands are conjugated to the oligonucleotide at the 5′ end of the oligonucleotide, the 3′ end of the oligonucleotide, one or more internal positions on the oligonucleotide, or any combination thereof. In certain embodiments, the oligonucleotide is a modified oligonucleotide.

In some embodiments, L₁, L₂, L₃, and L₄ together comprise the structure:

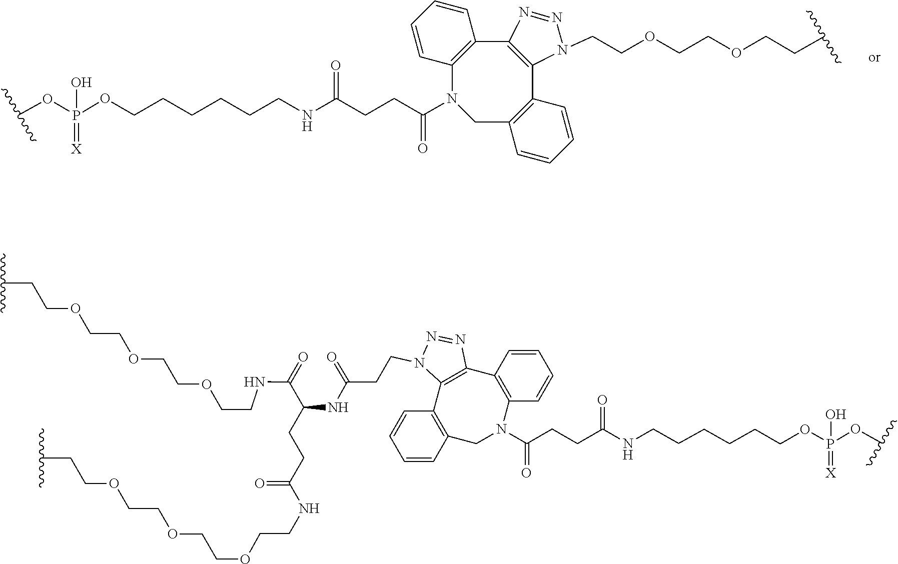

wherein X is O or S.

In certain embodiments, L₁, L₂. L₃, and L₄ together comprise the structure:

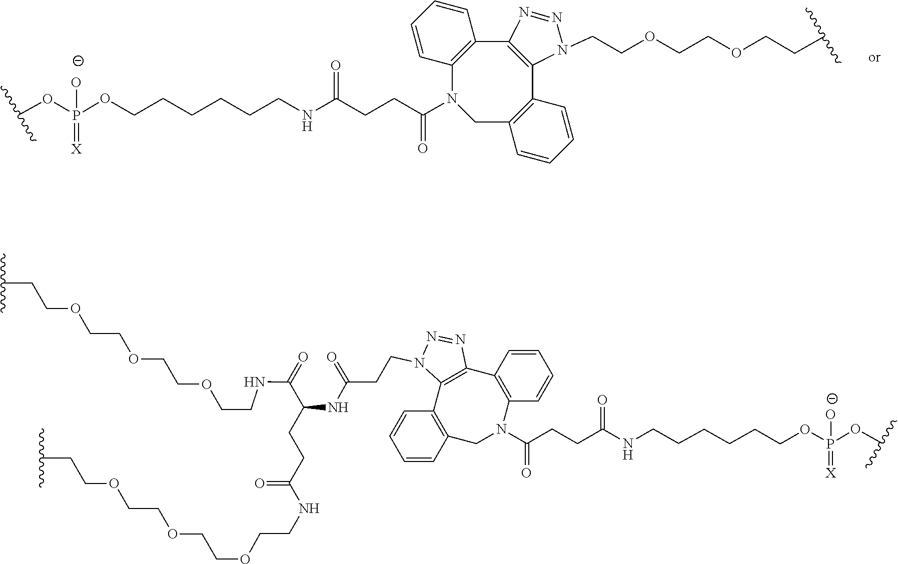

wherein X is O or S.

In some embodiments, L₁, L₂, L₃, L₄, L₅, L₆, and L₇ together comprise the structure:

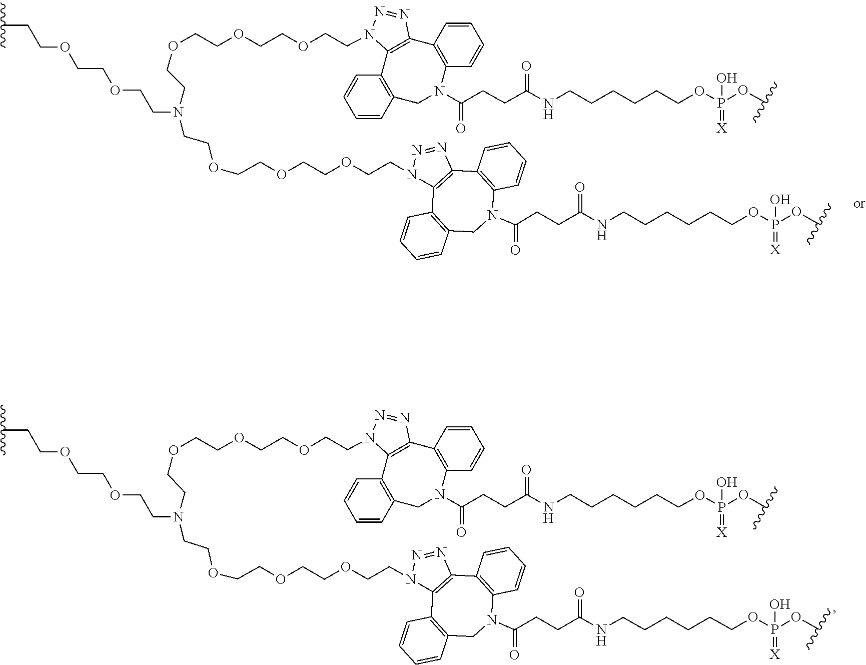

wherein X is O or S.

In certain embodiments, L₁, L₂, L₃, L₄, L₅, L₆, and L₇ together comprise the structure:

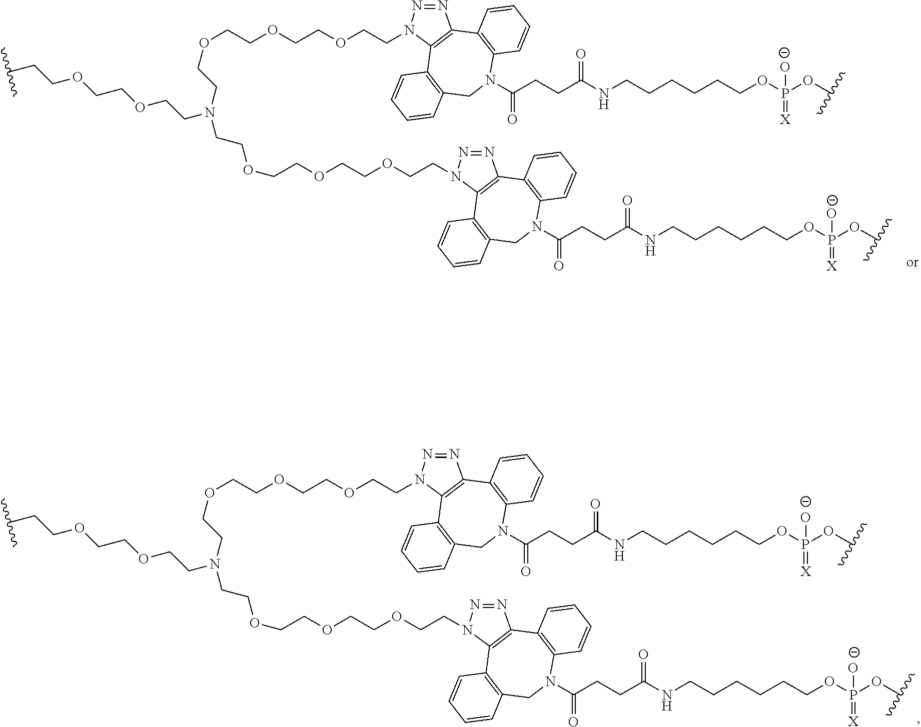

wherein X is O or S.

In some aspects, the present disclosure provides compounds, and stereoisomers, tautomers, prodrugs, and salts thereof, comprising the structure:

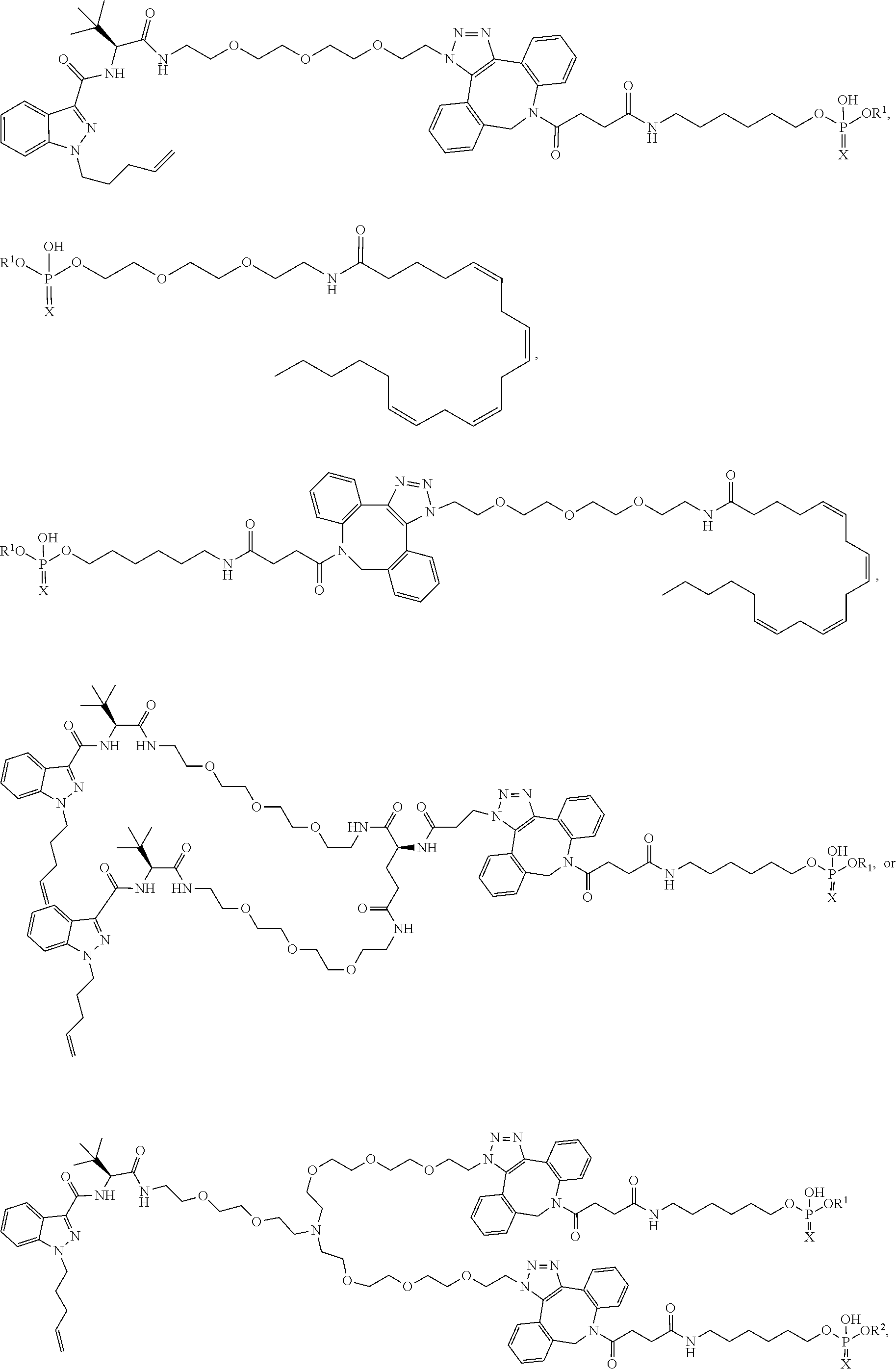

wherein:
R¹ and R² each independently comprise one or more oligonucleotides, protecting groups, small molecules, proteins, antibodies, and/or peptides; and
X is O or S.

In some embodiments, the present disclosure provides compounds comprising the structure:

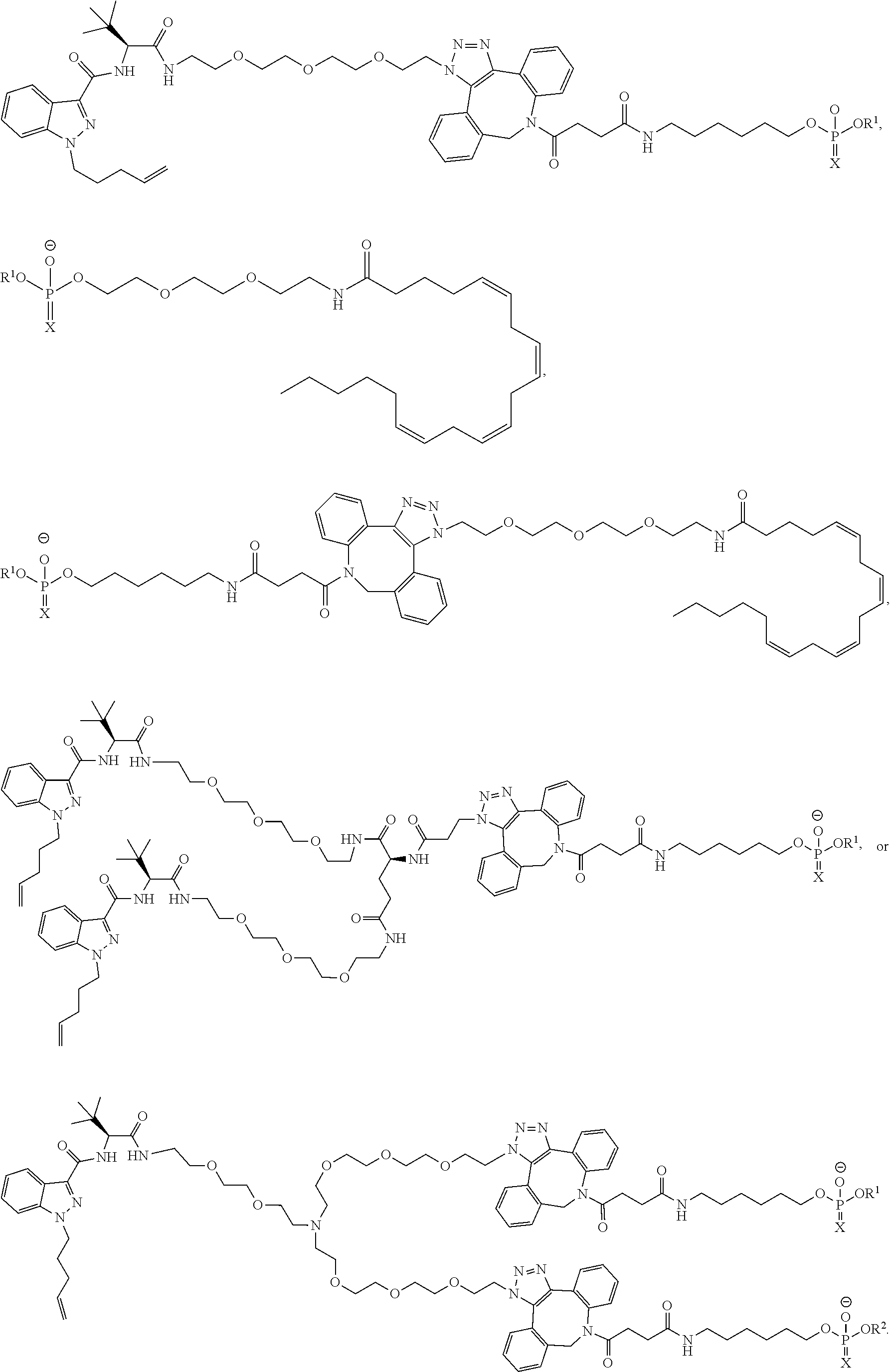

In some aspects, the present disclosure provides compounds, and stereoisomers, tautomers, prodrugs, and salts thereof, comprising the structure:

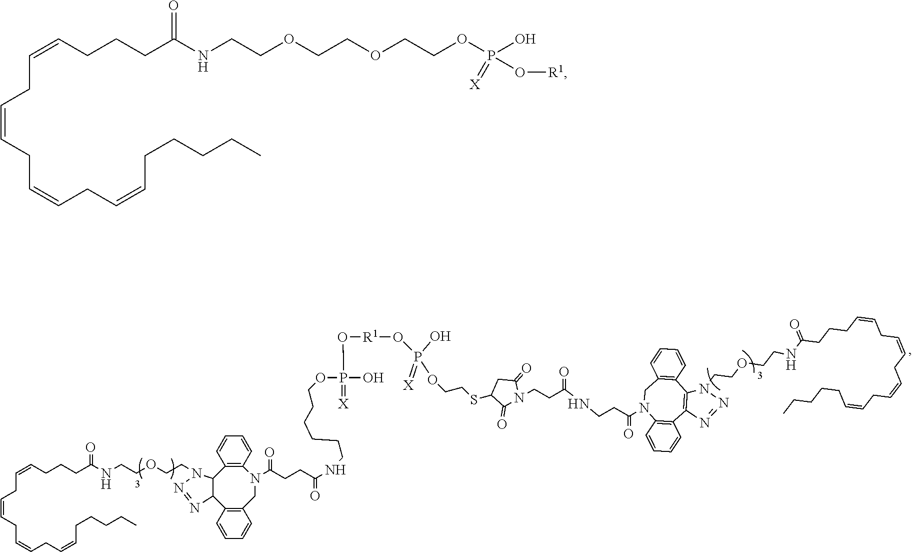

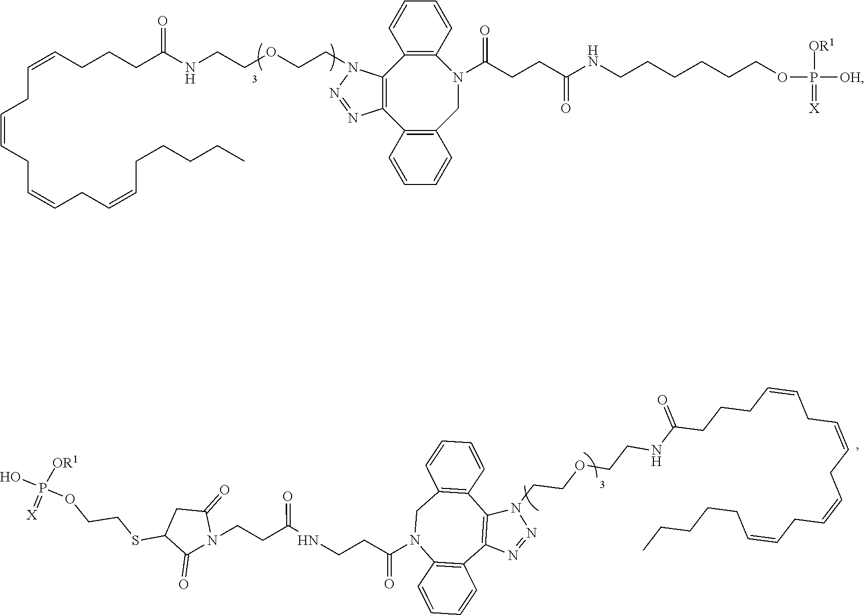

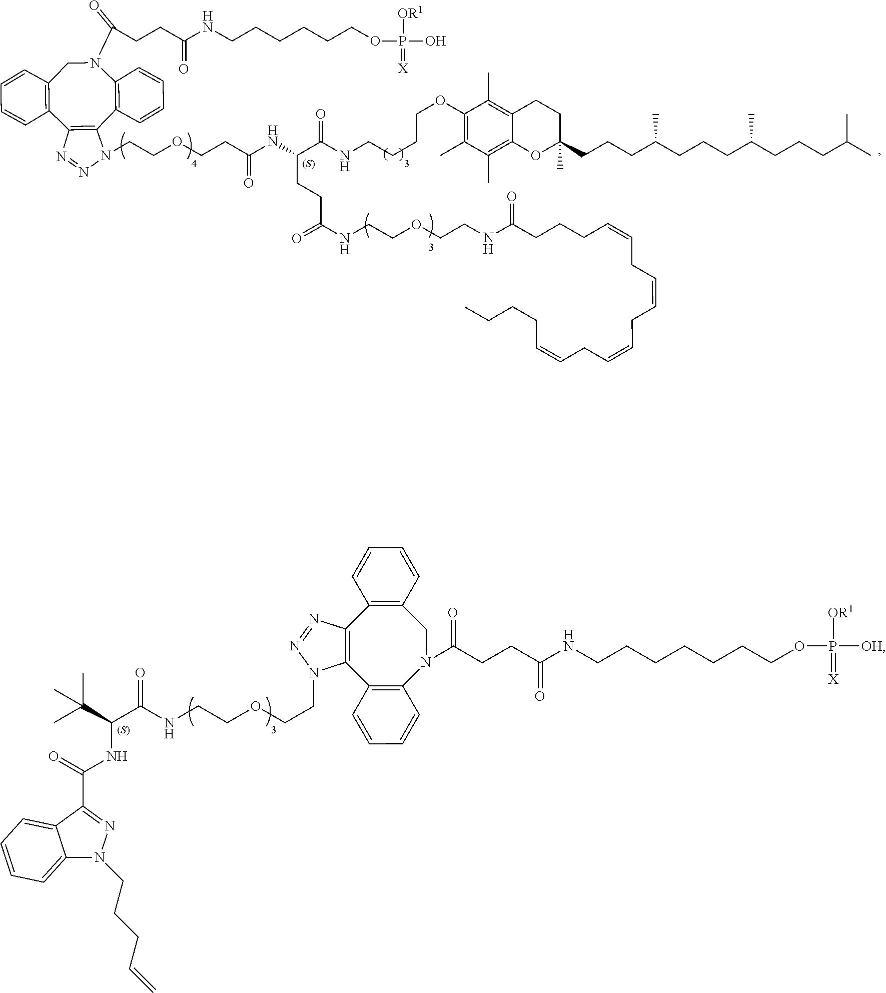

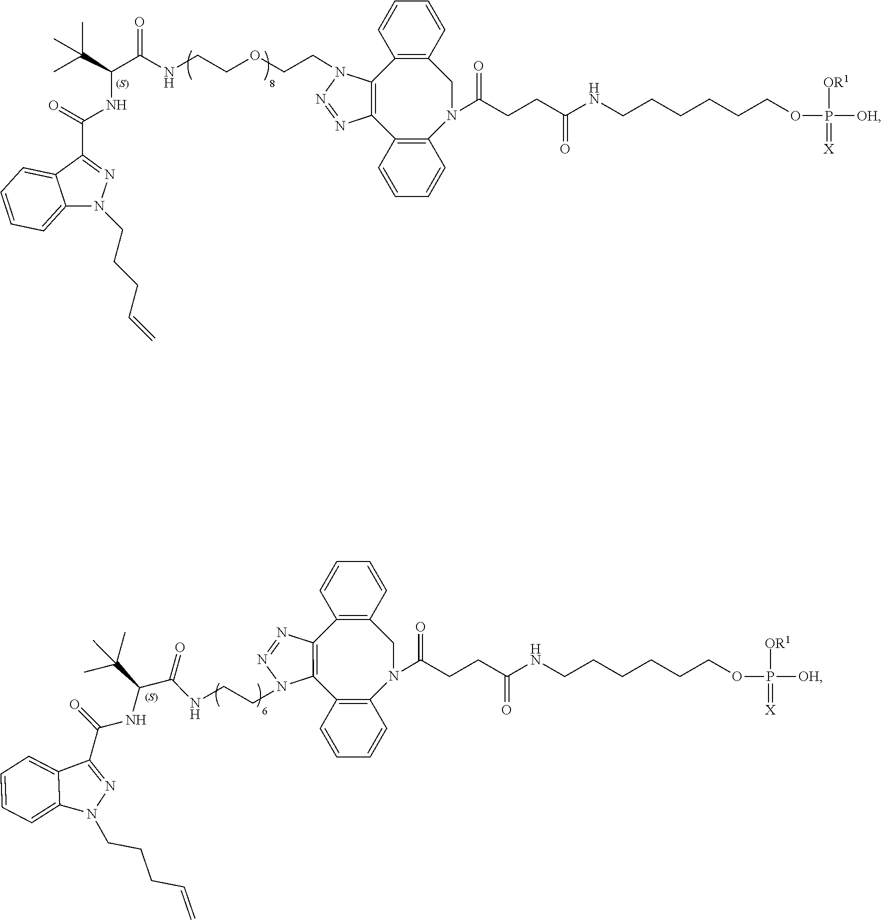

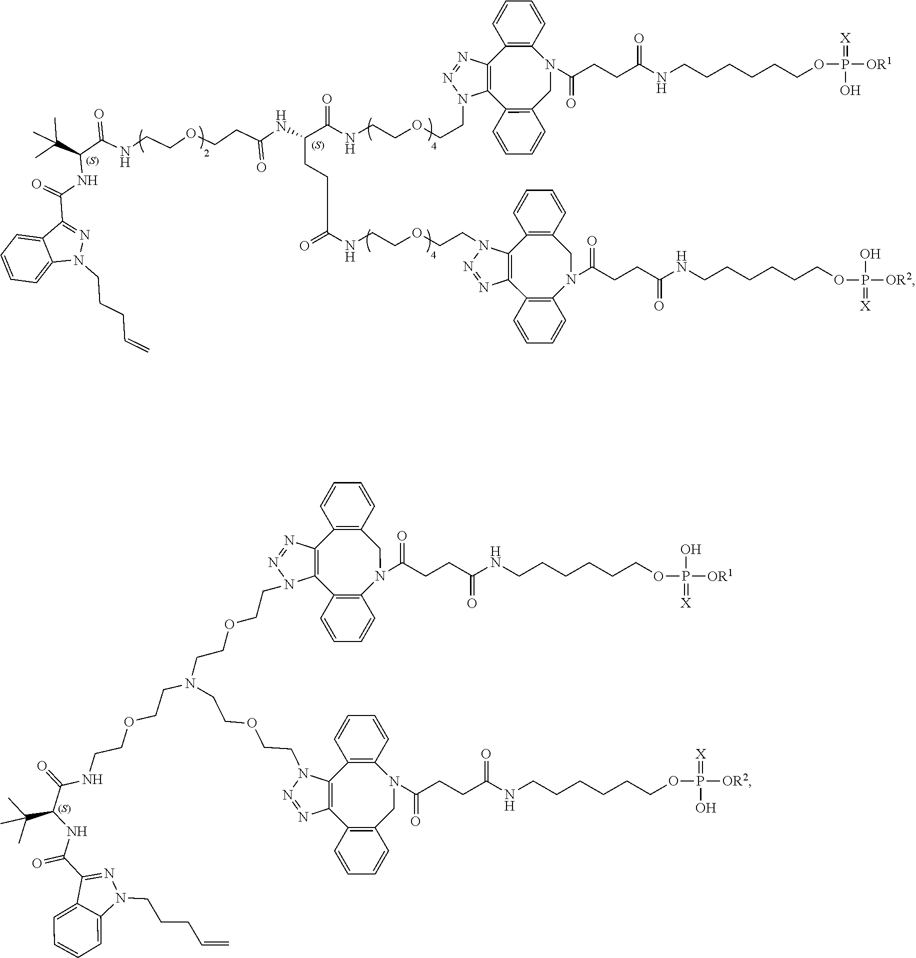

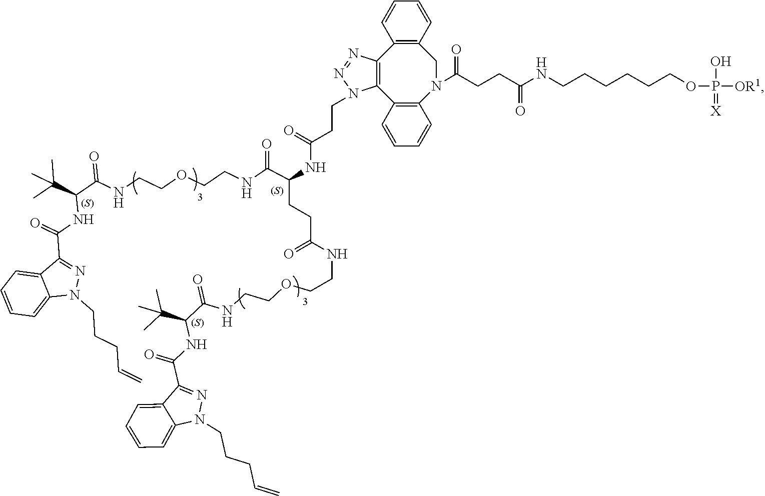

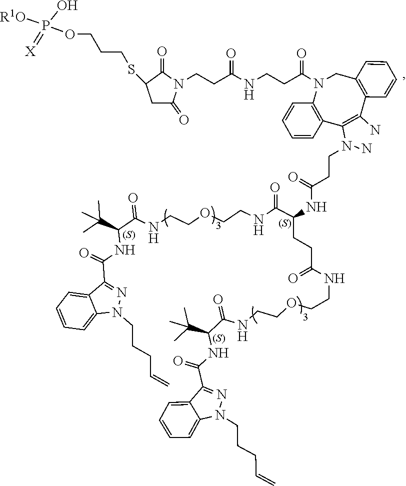

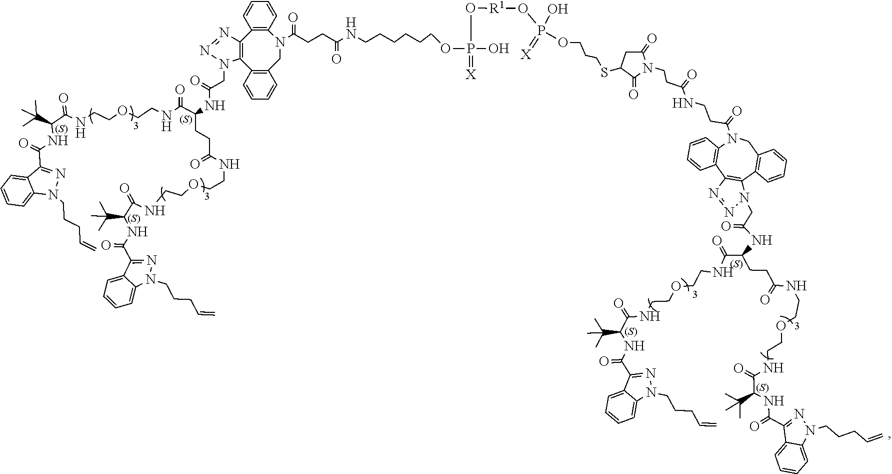

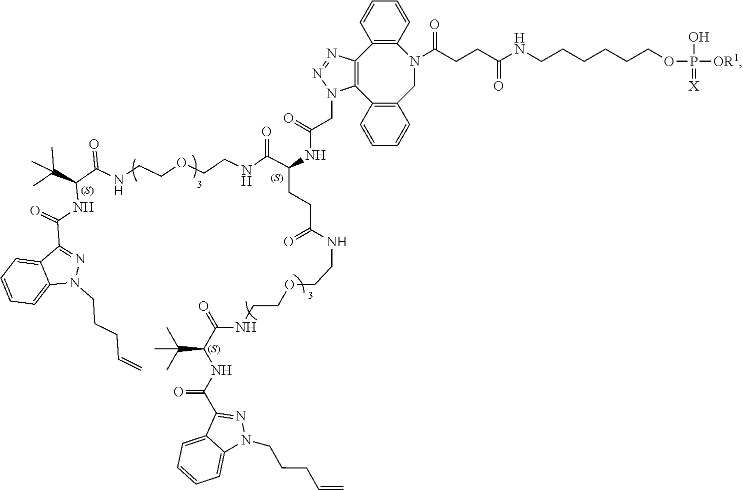

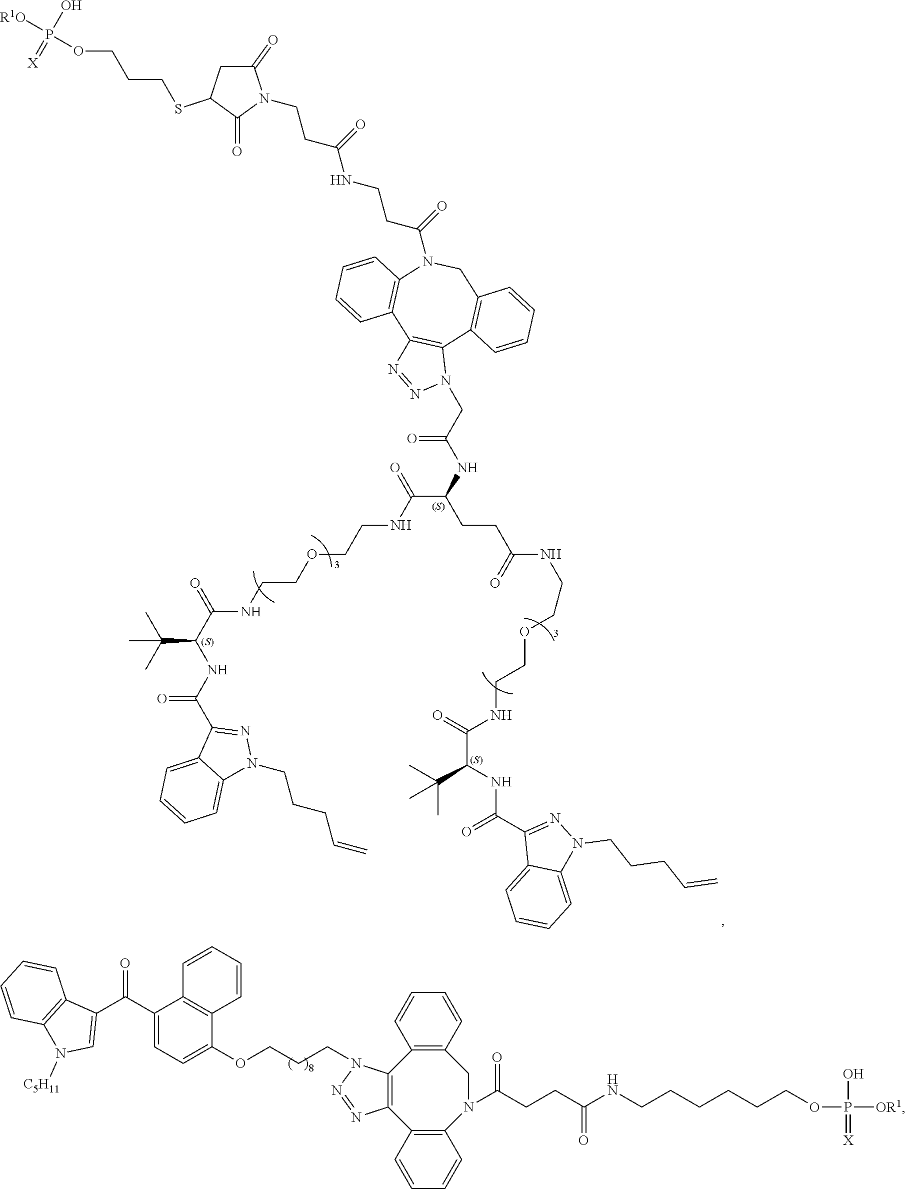

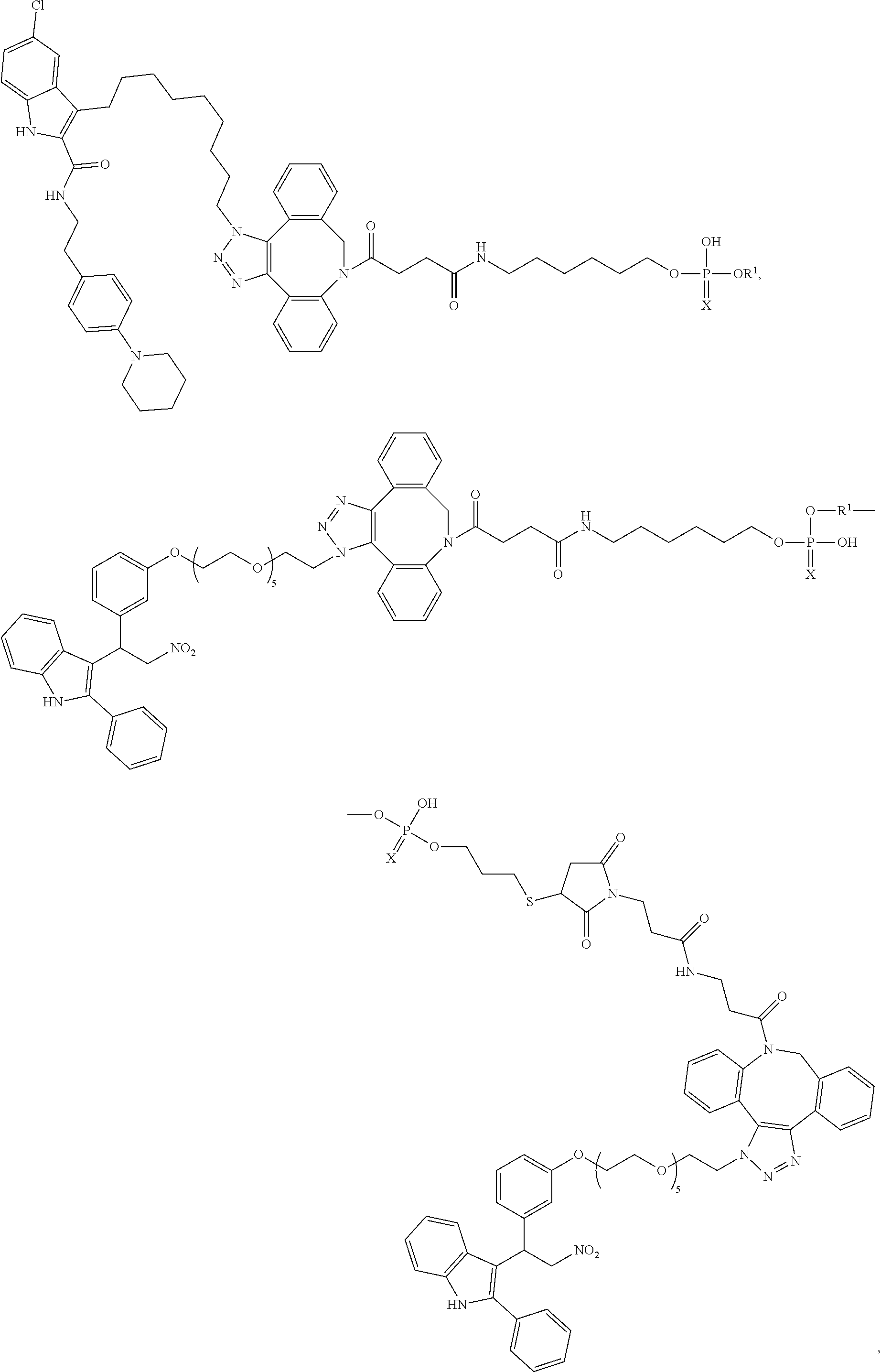

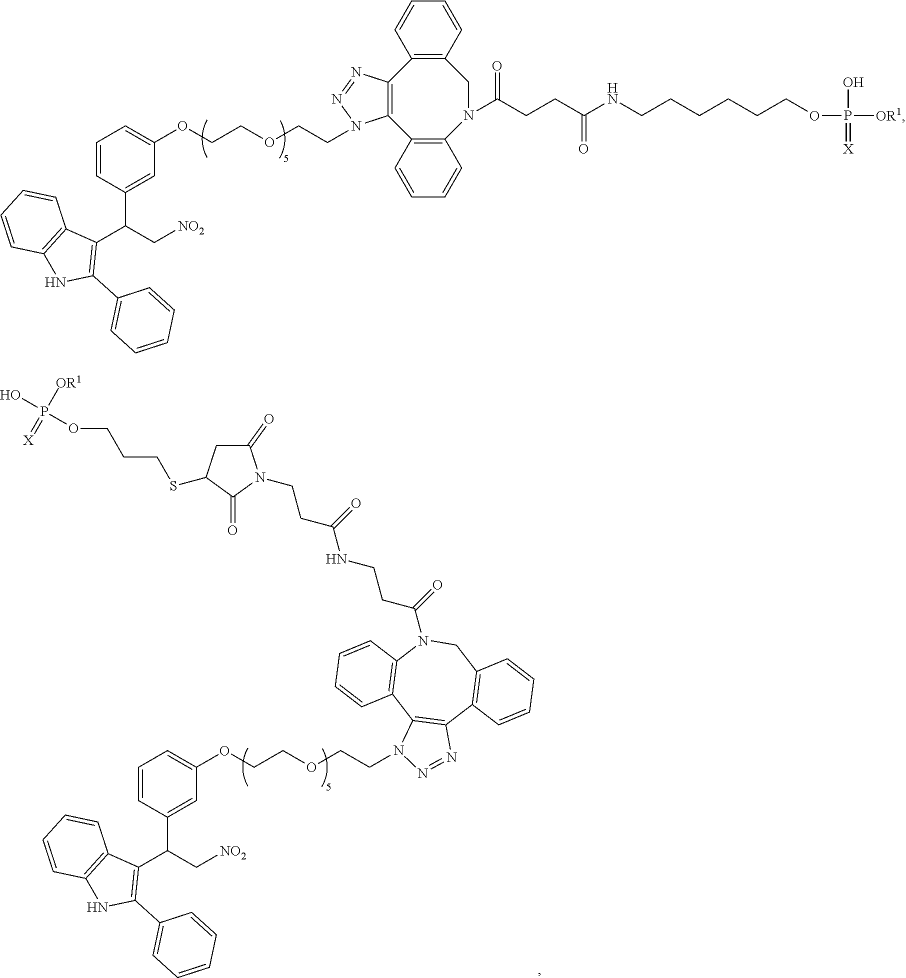

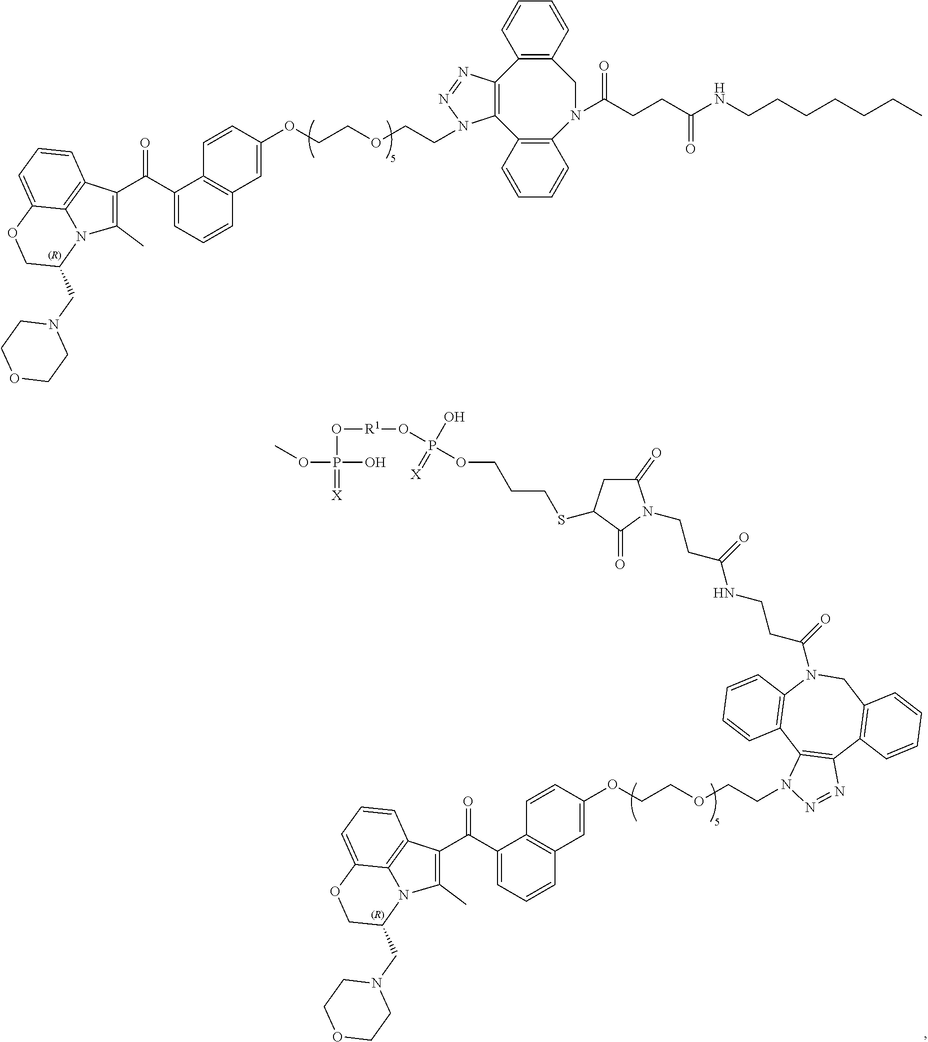

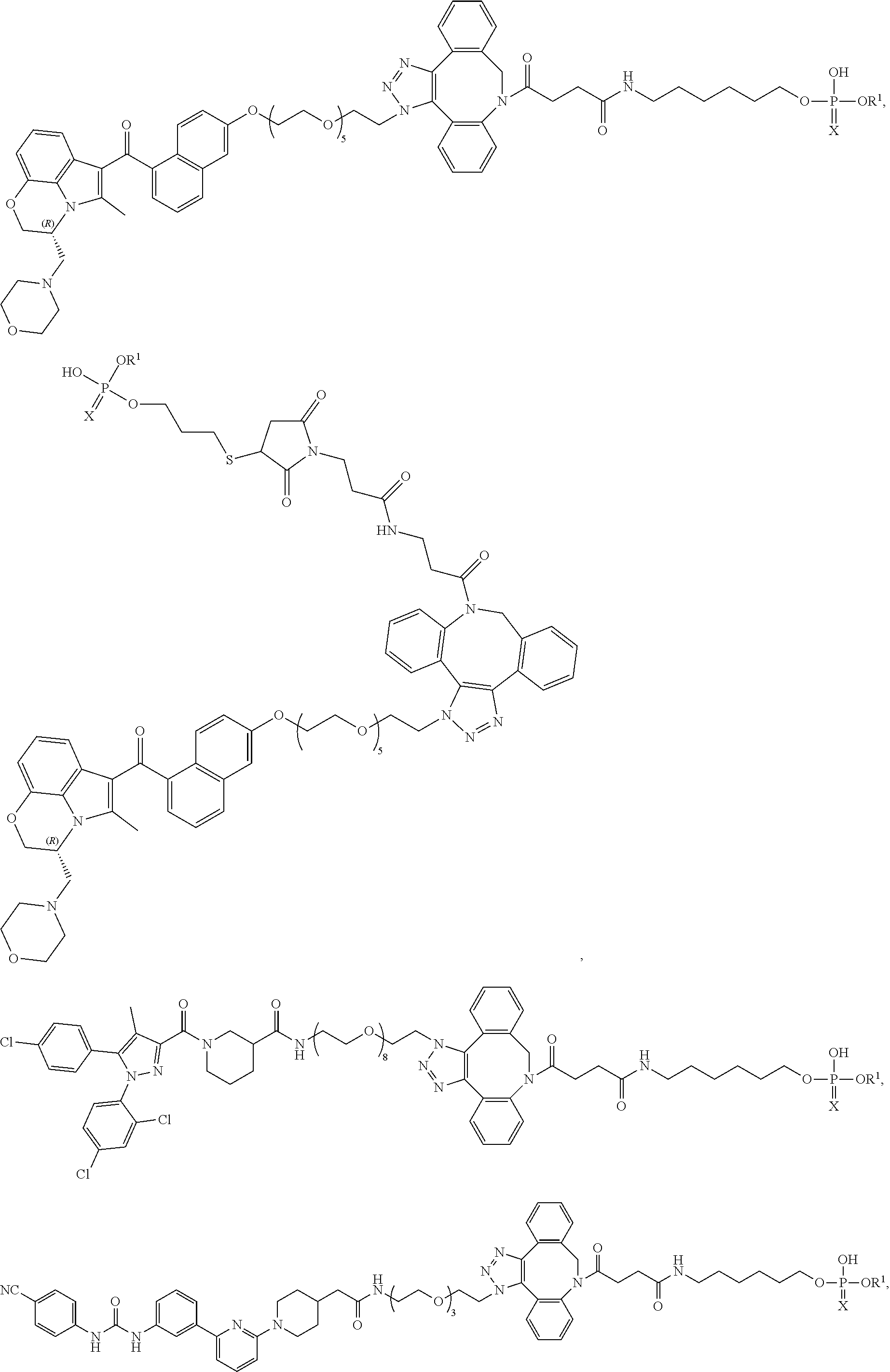

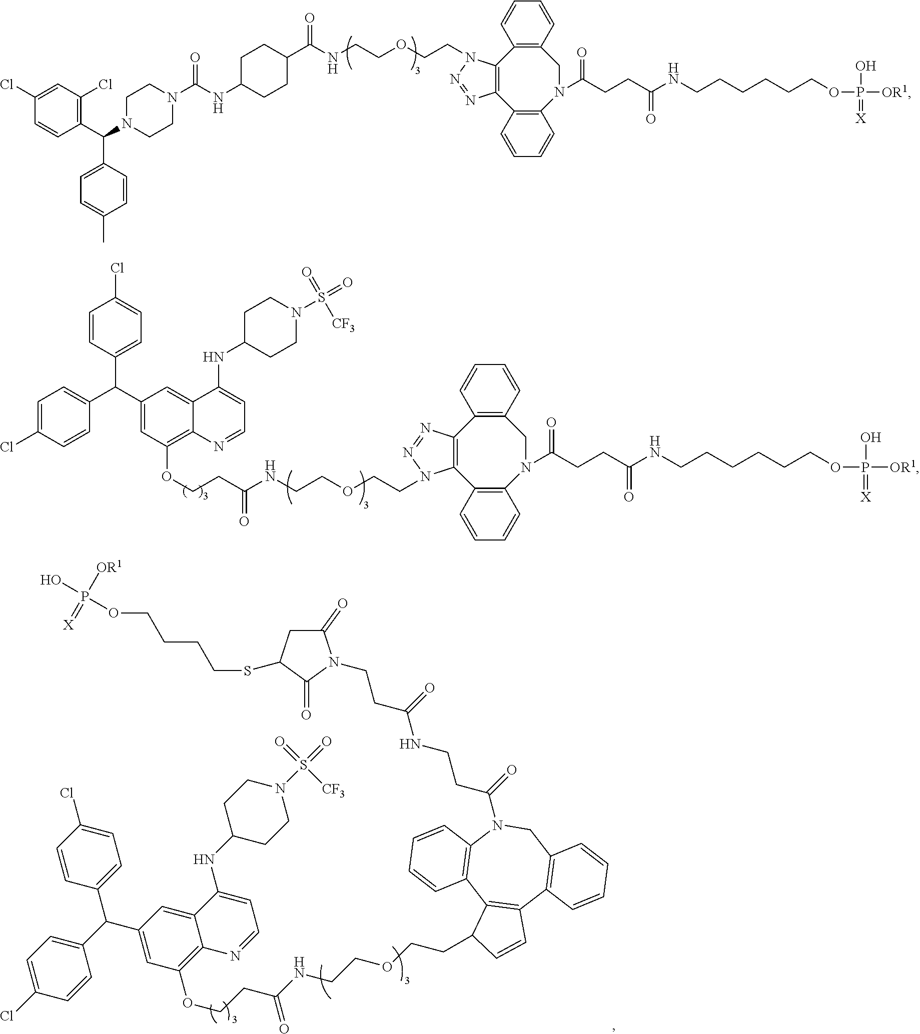

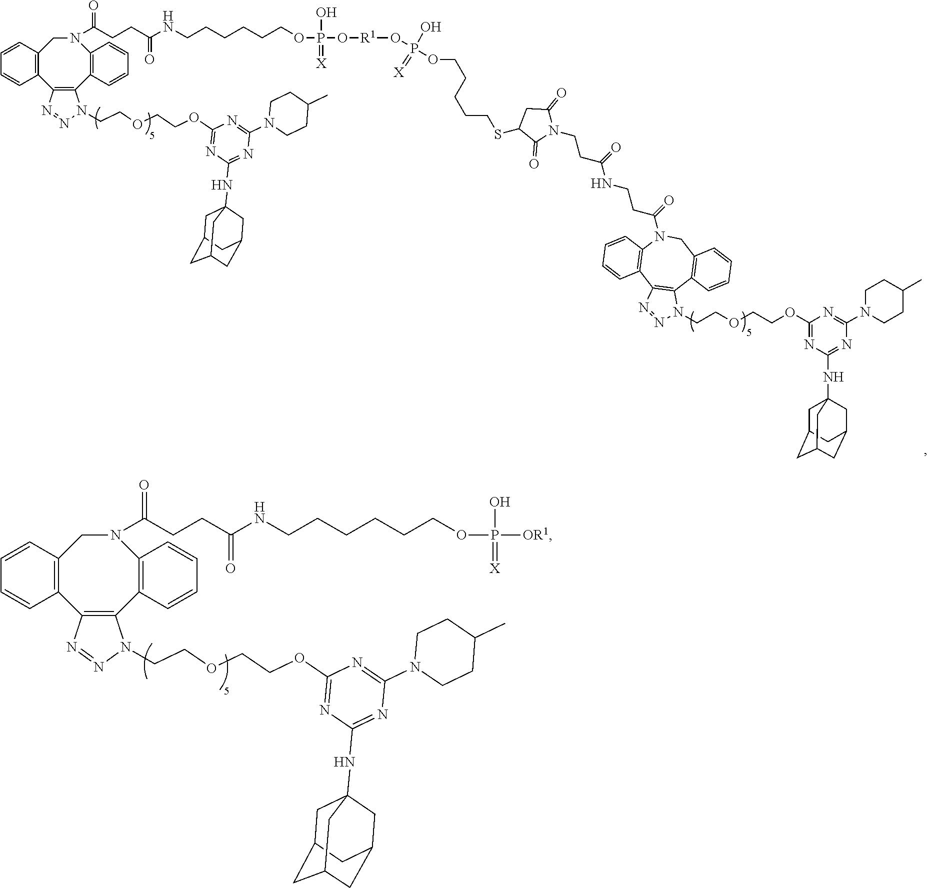

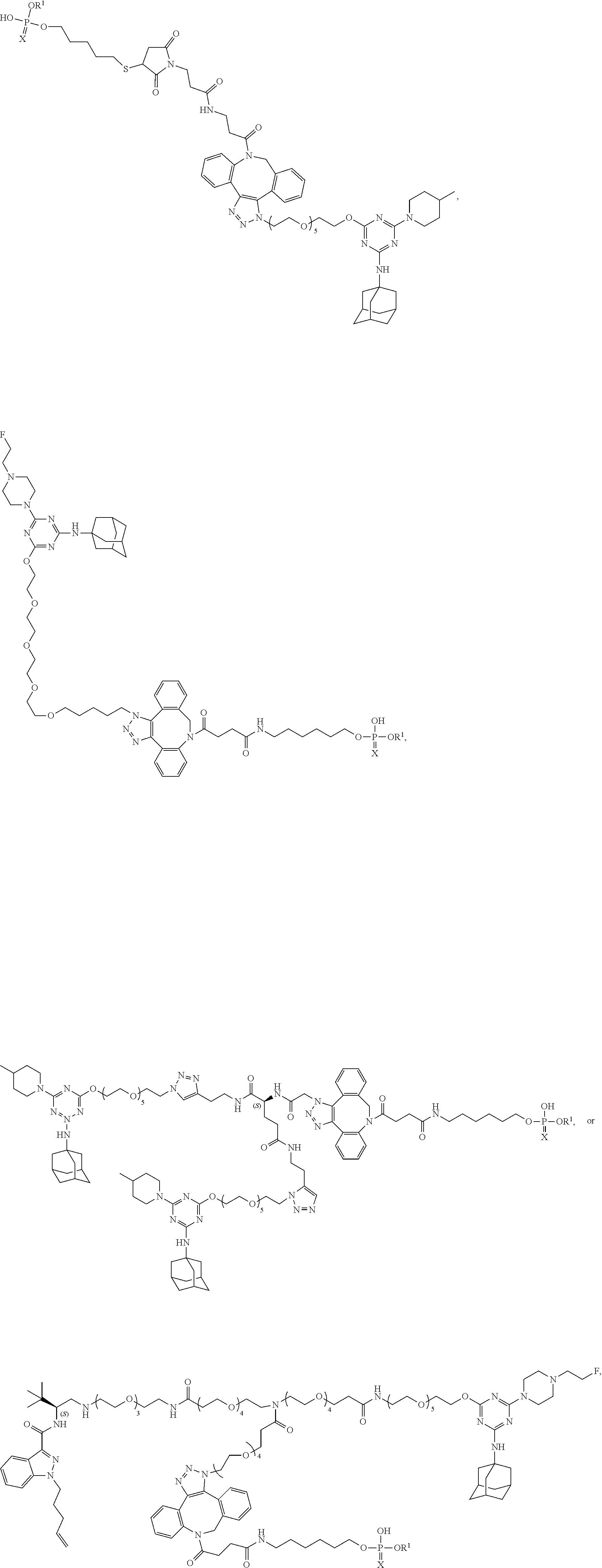

wherein:
R¹ and R² each independently comprise one or more oligonucleotides, protecting groups, small molecules, proteins, antibodies, and/or peptides; and
X is O or S.

In certain embodiments, the present disclosure provides compounds, and stereoisomers, tautomers, prodrugs, and salts thereof, comprising the structure:

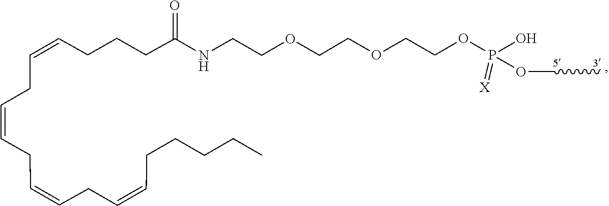

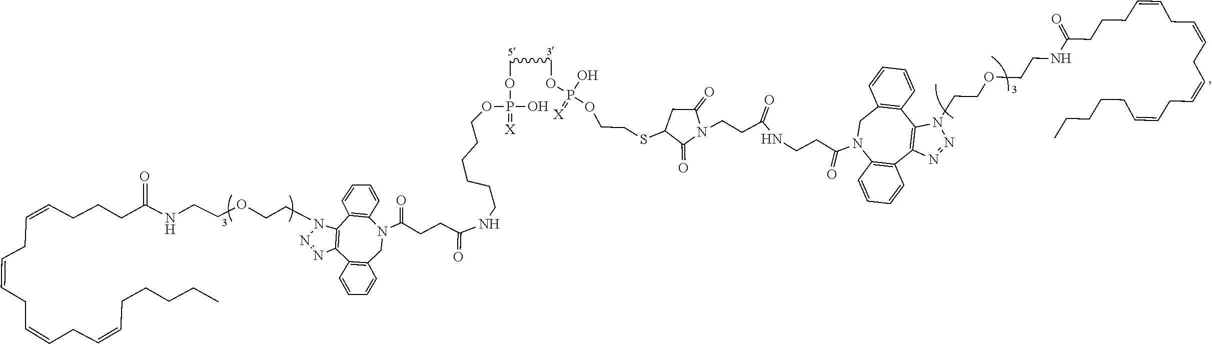

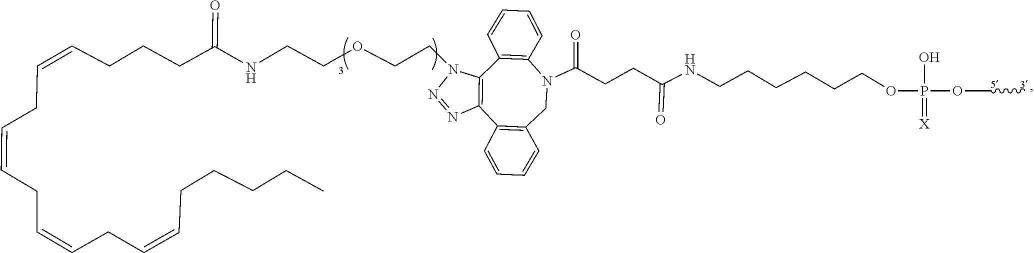

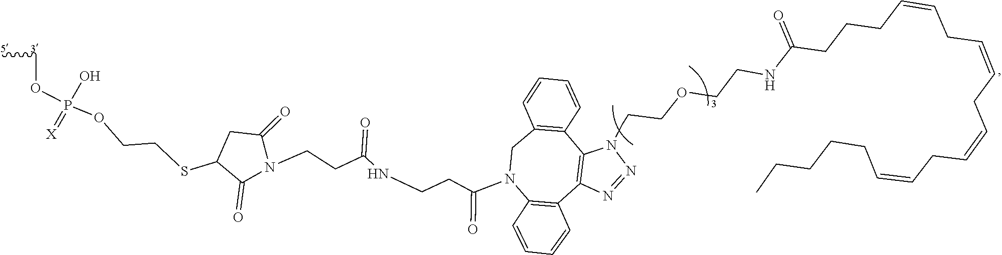

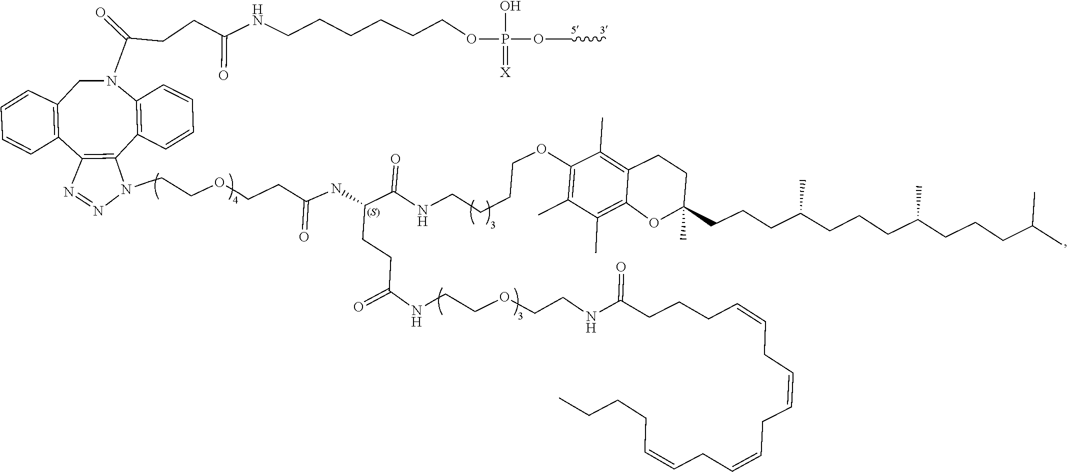

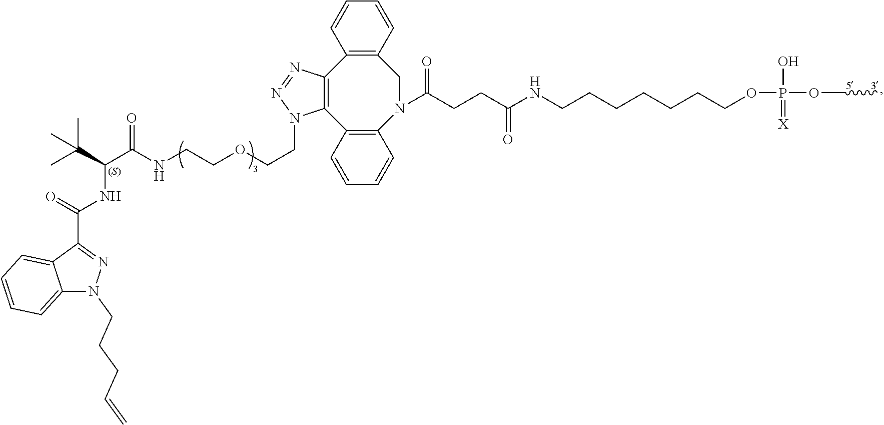

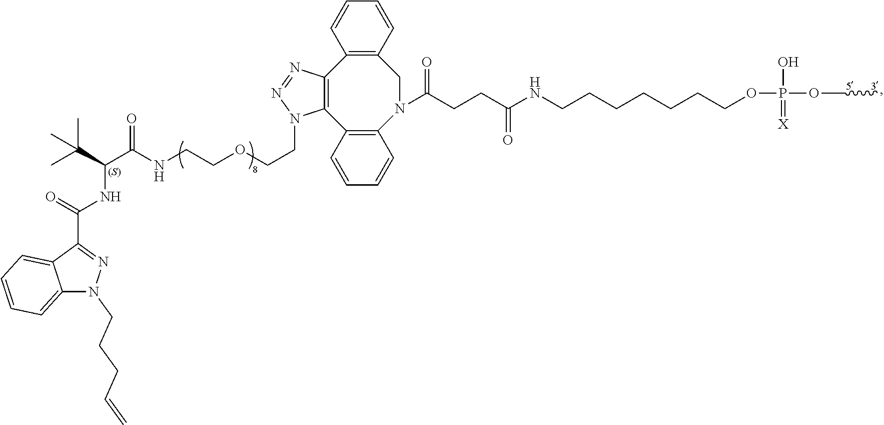

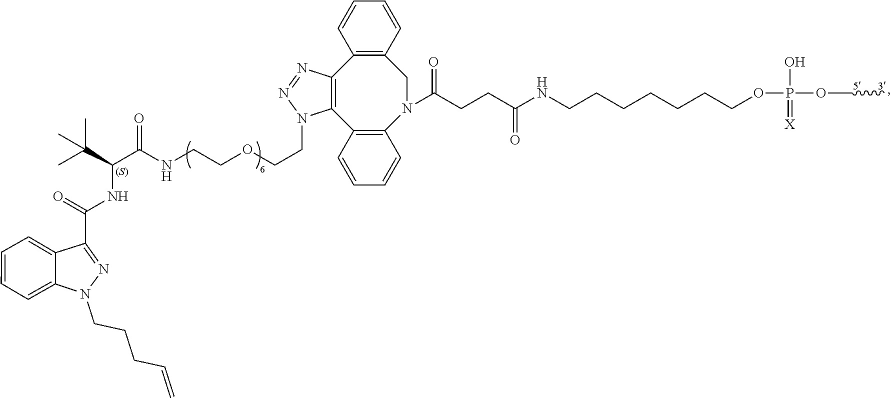

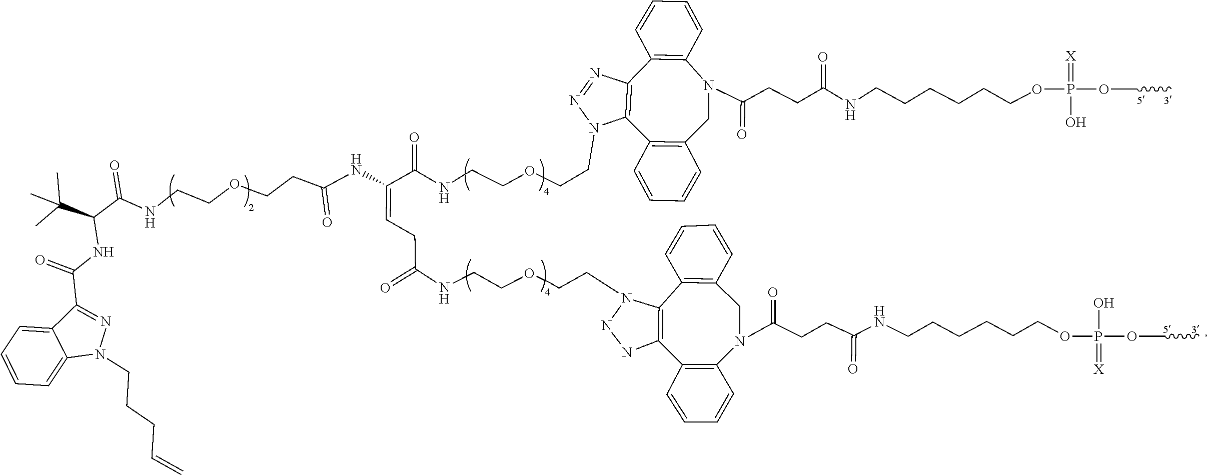

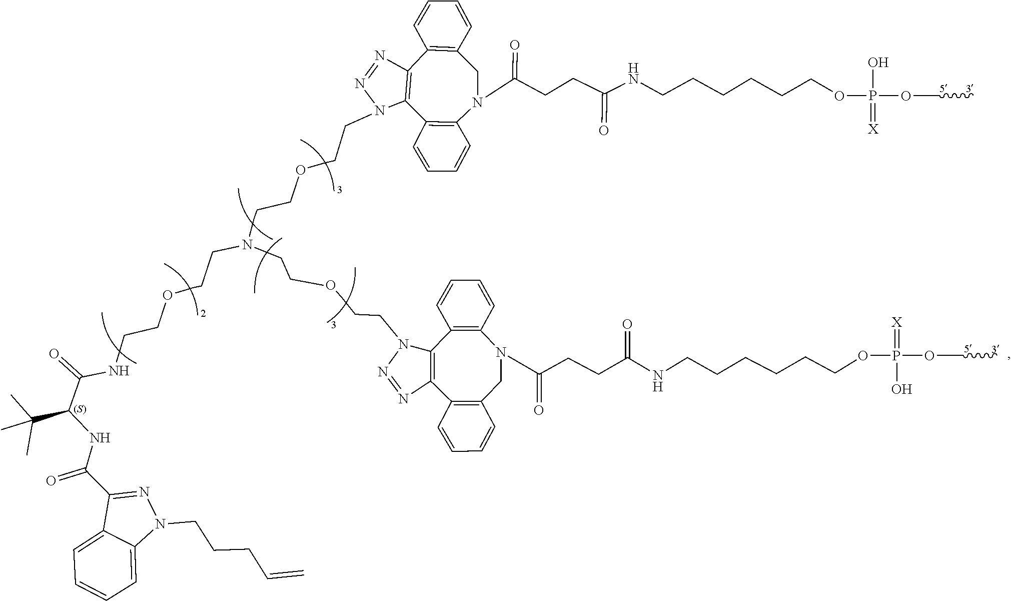

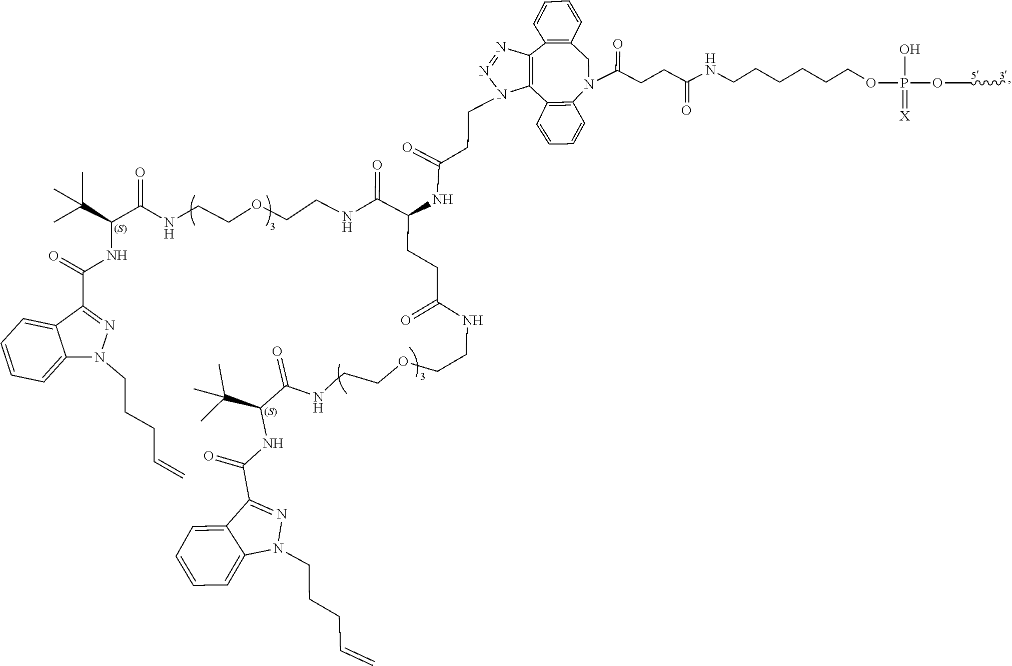

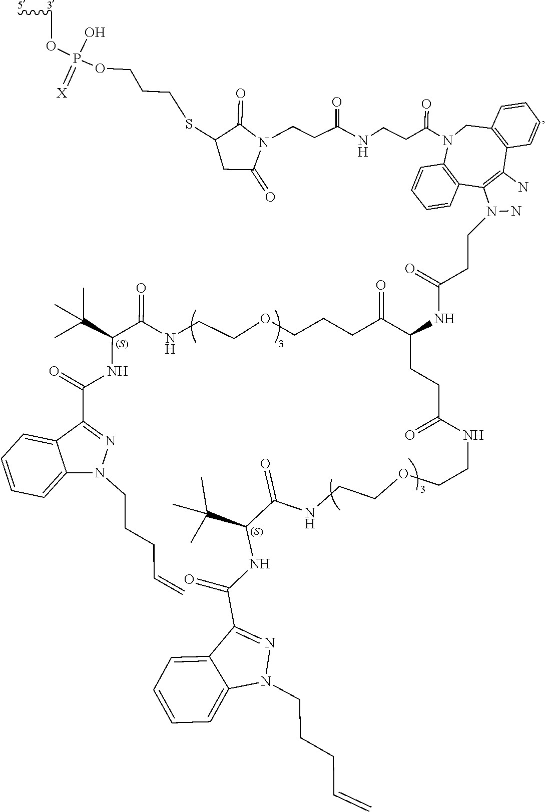

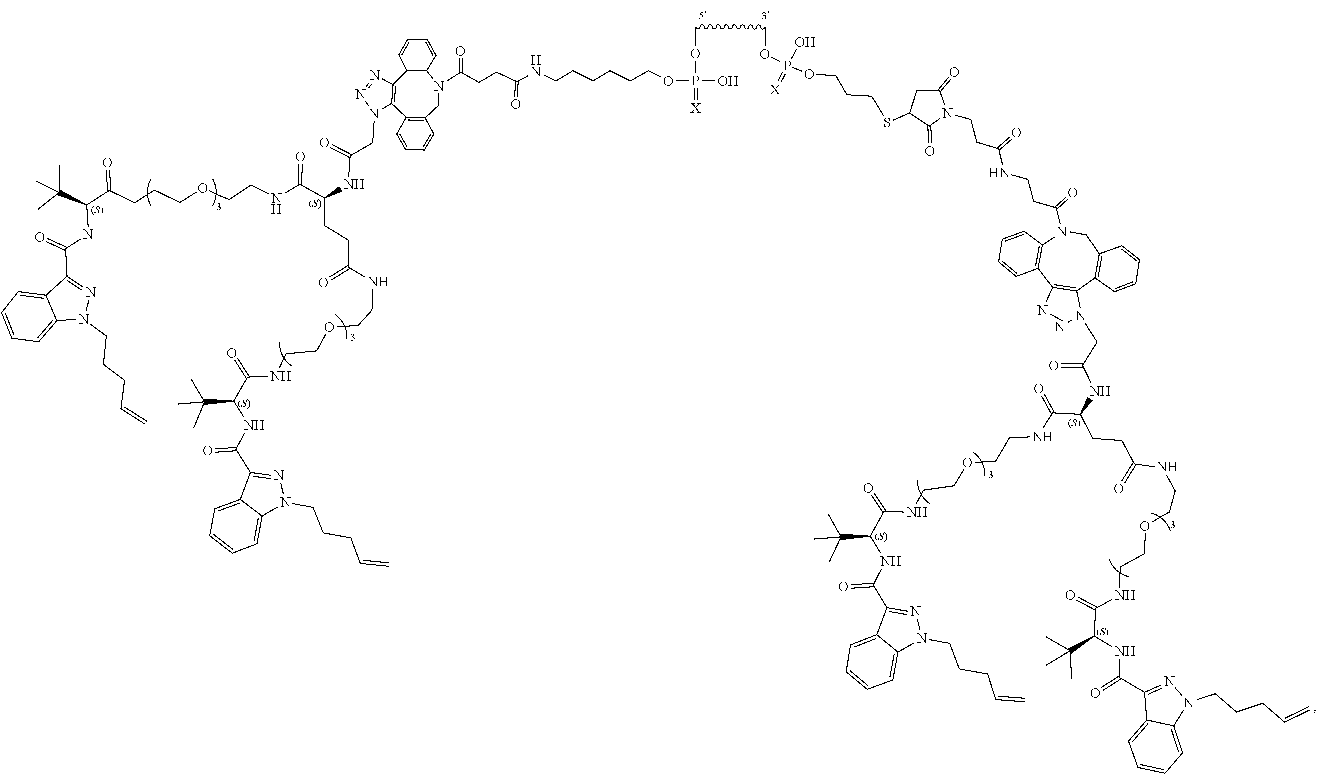

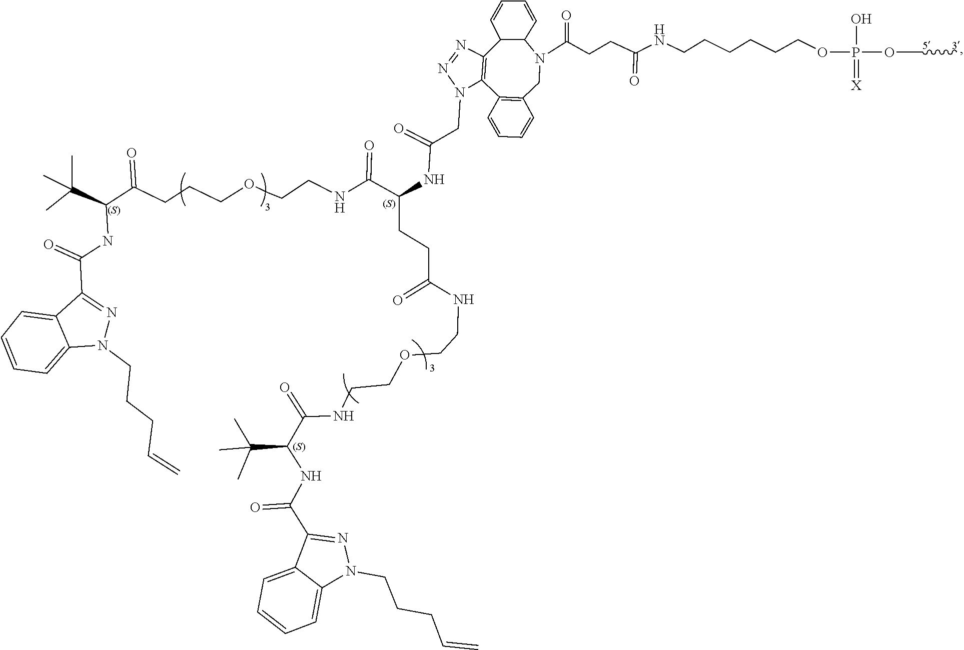

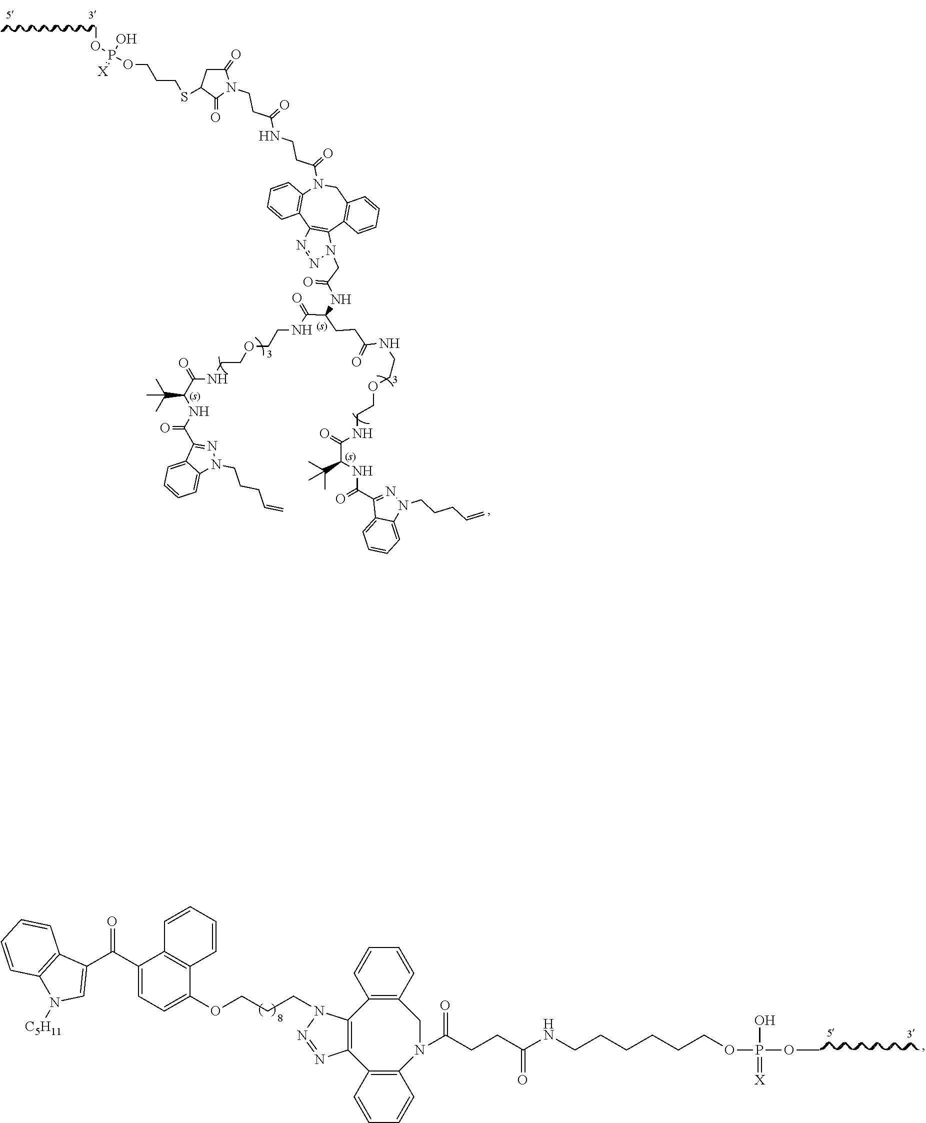

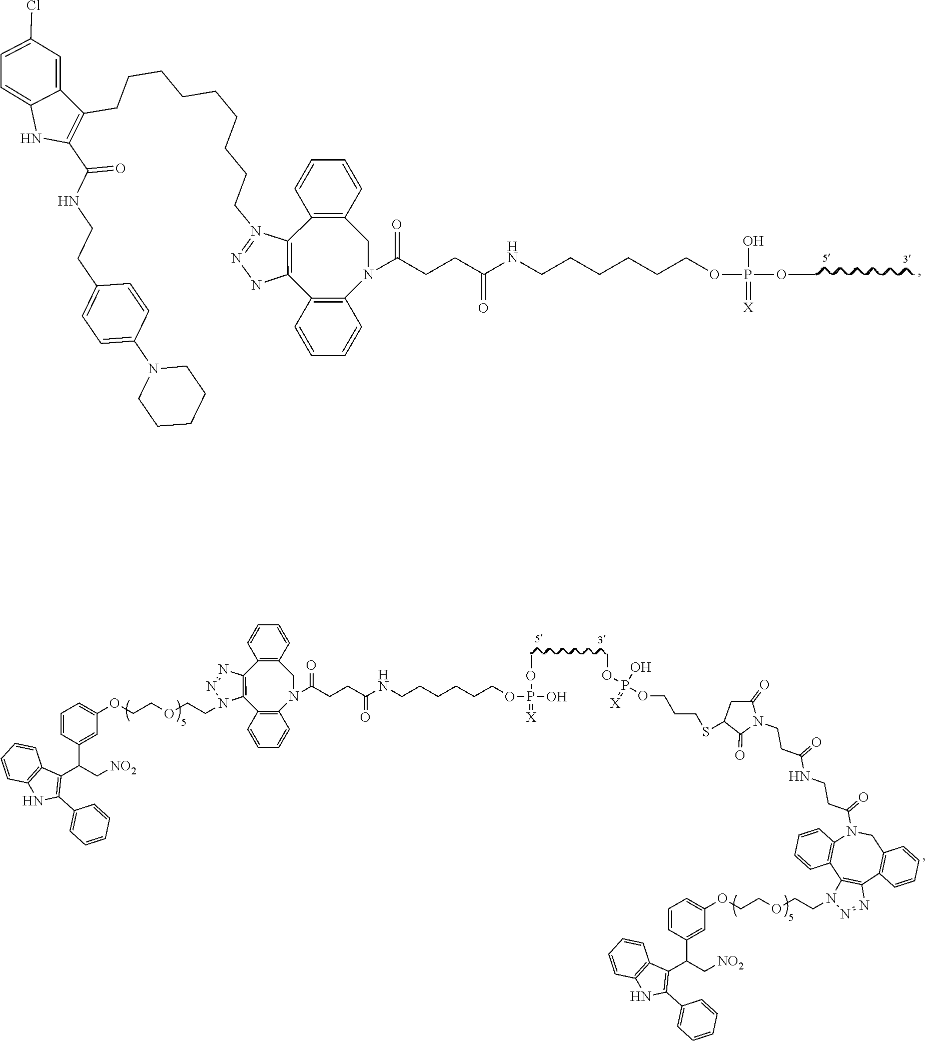

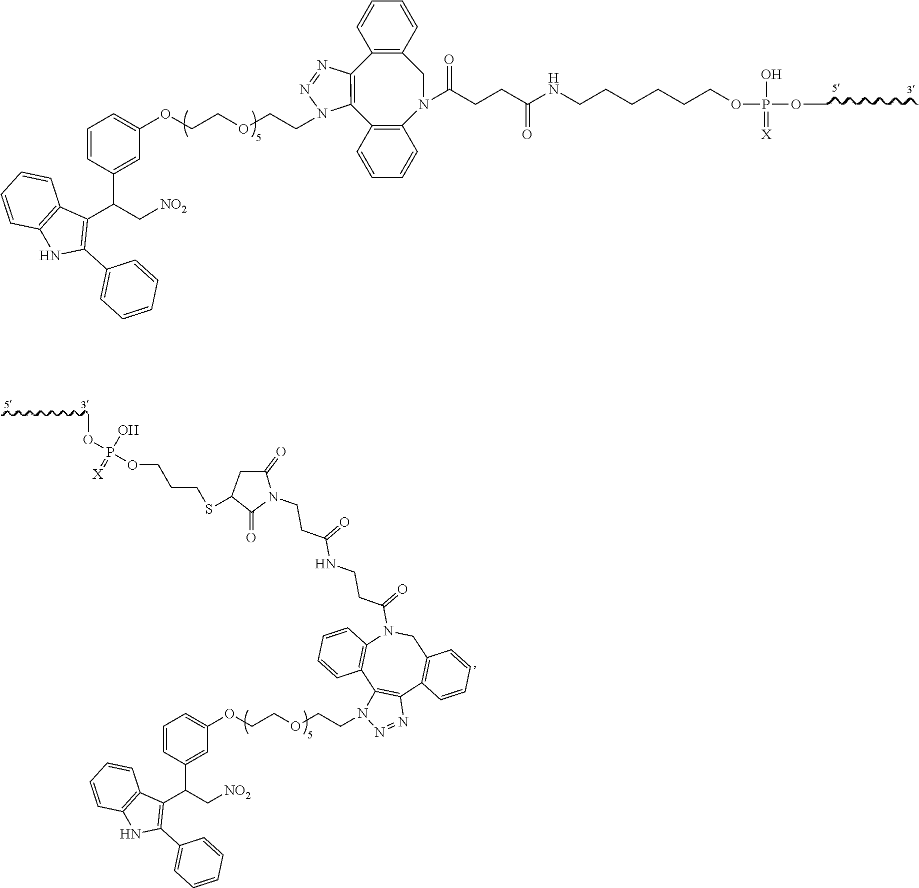

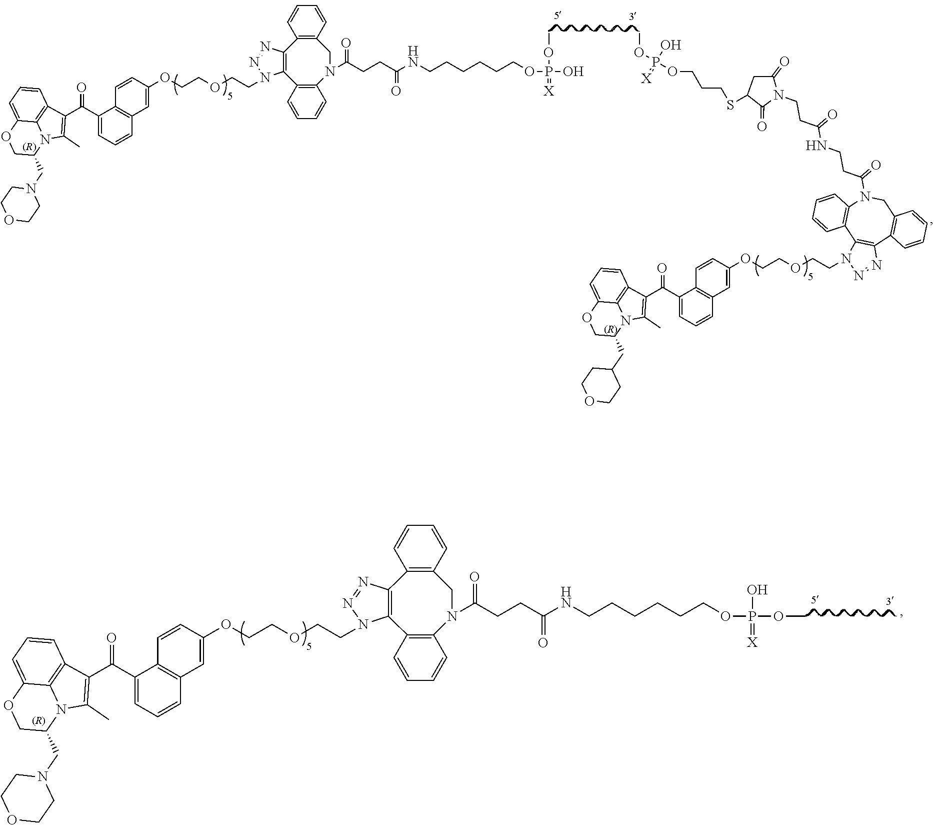

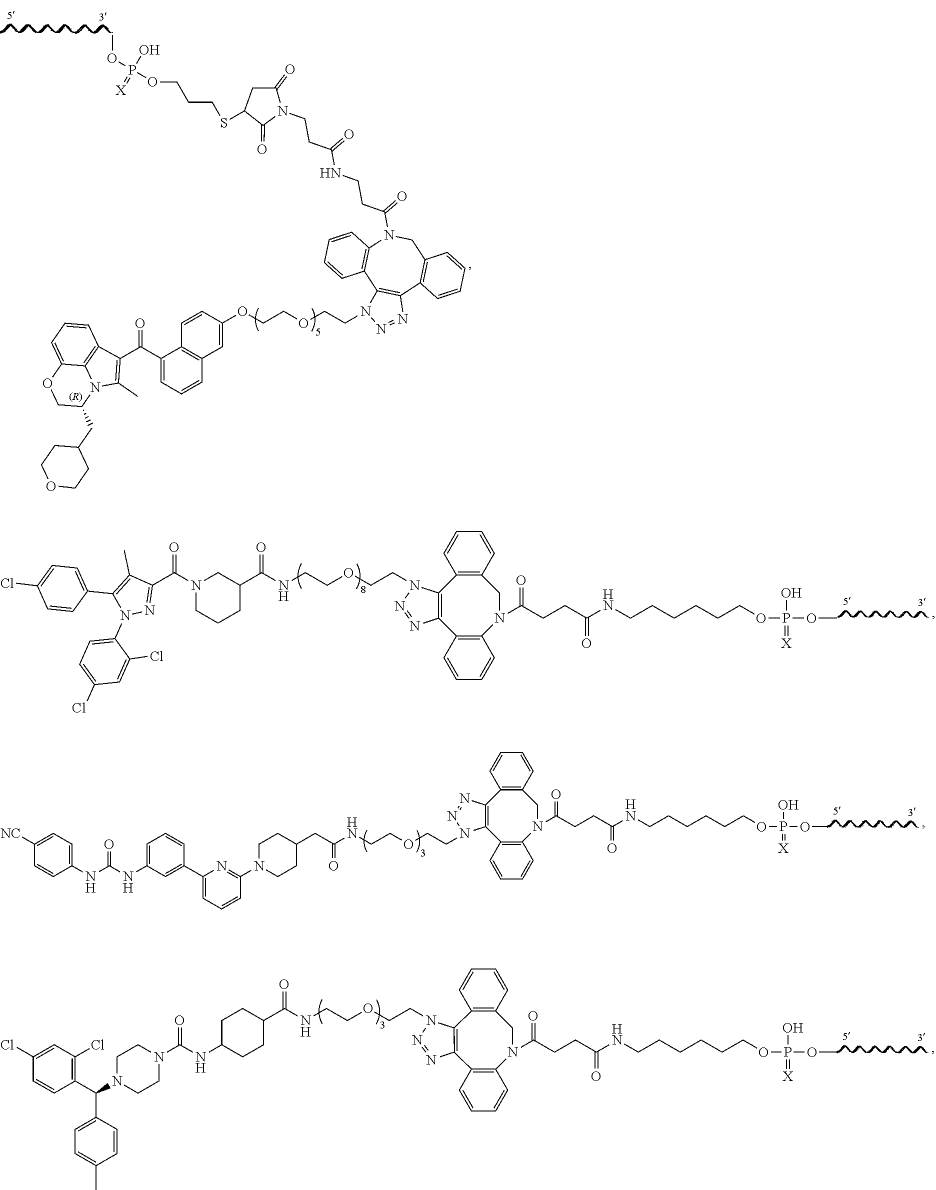

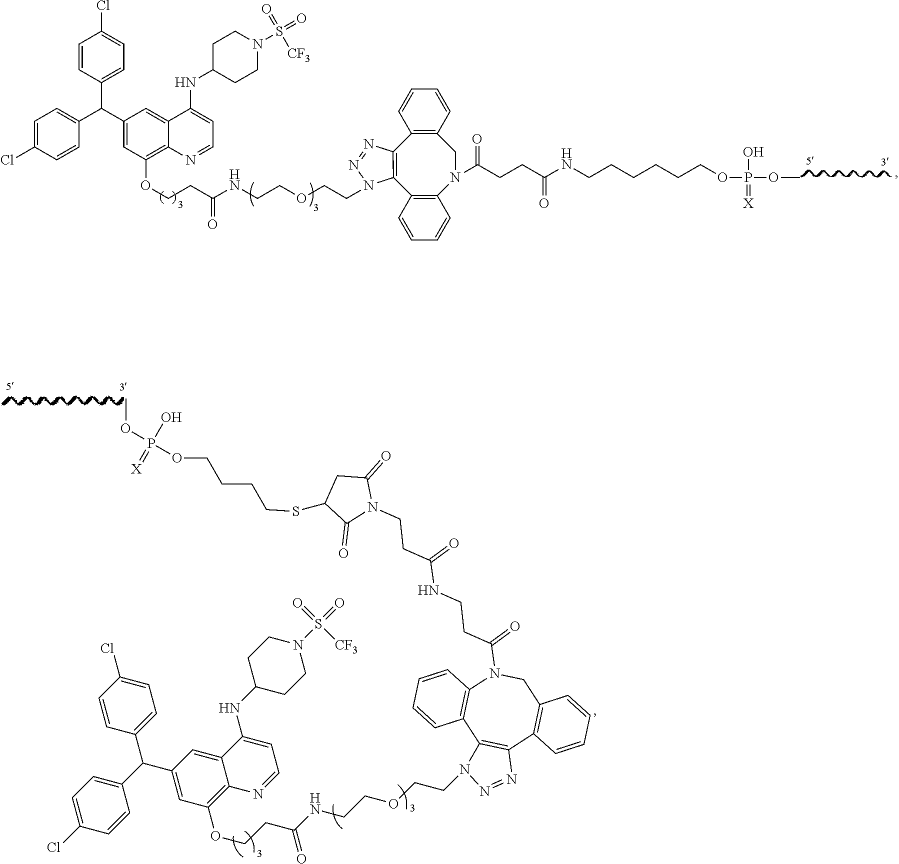

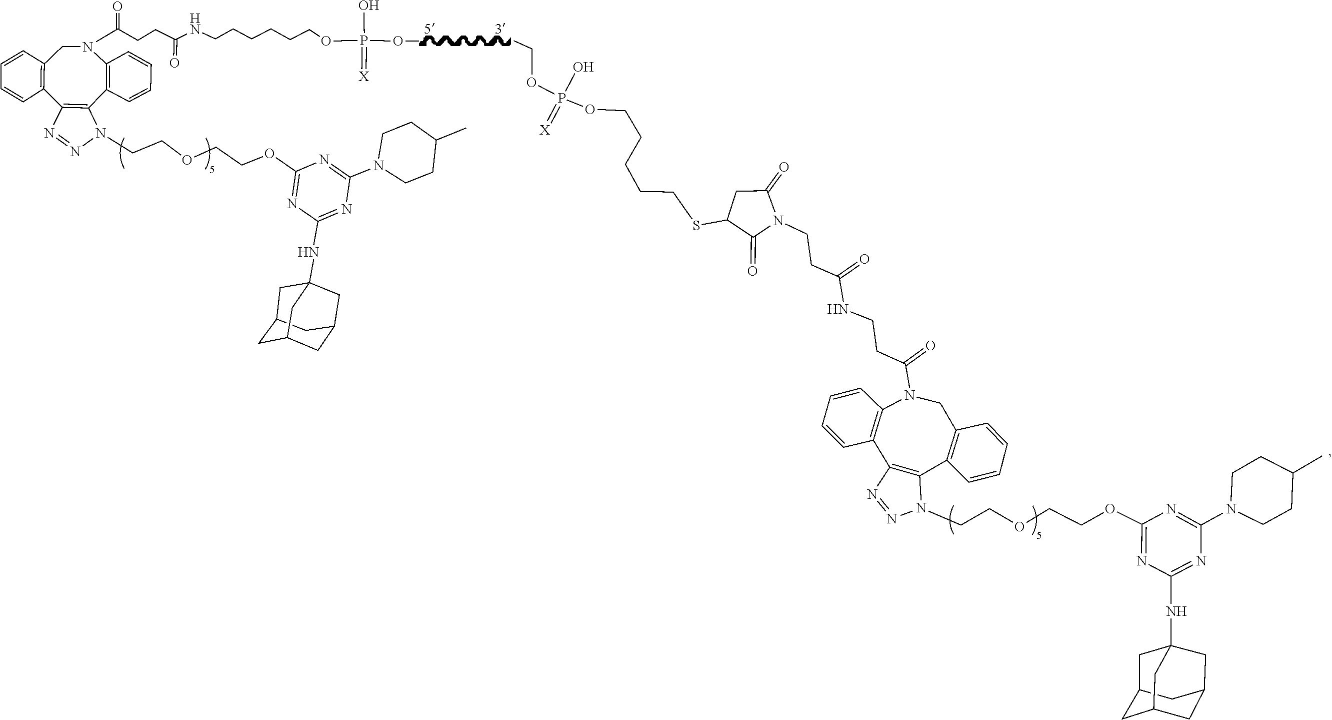

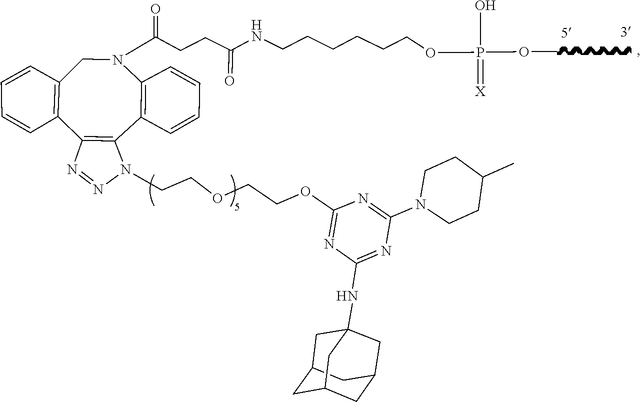

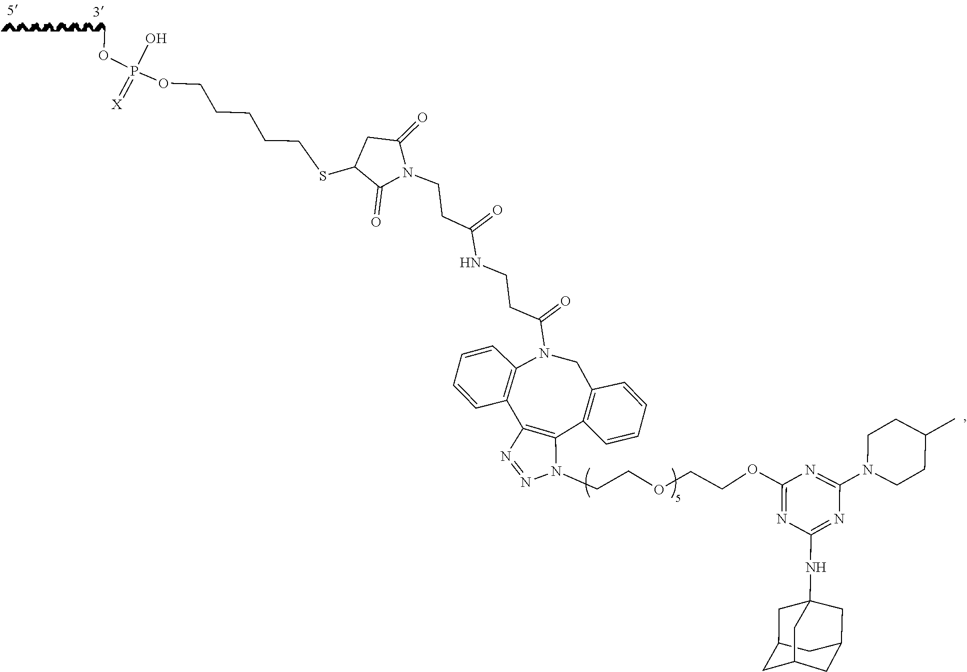

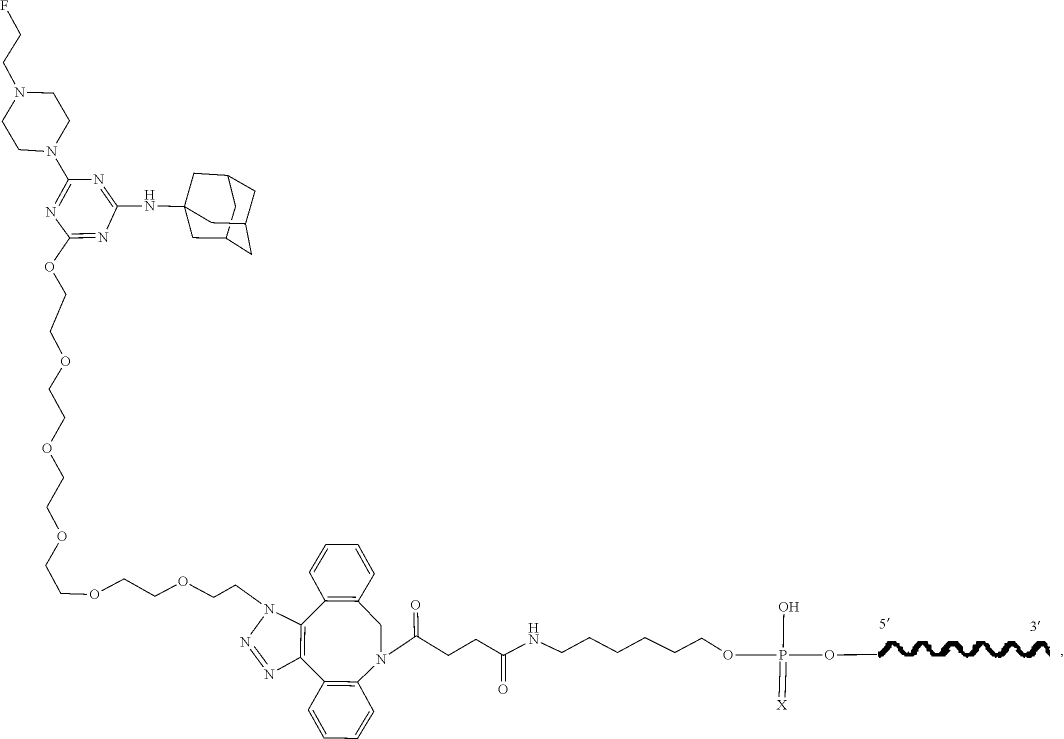

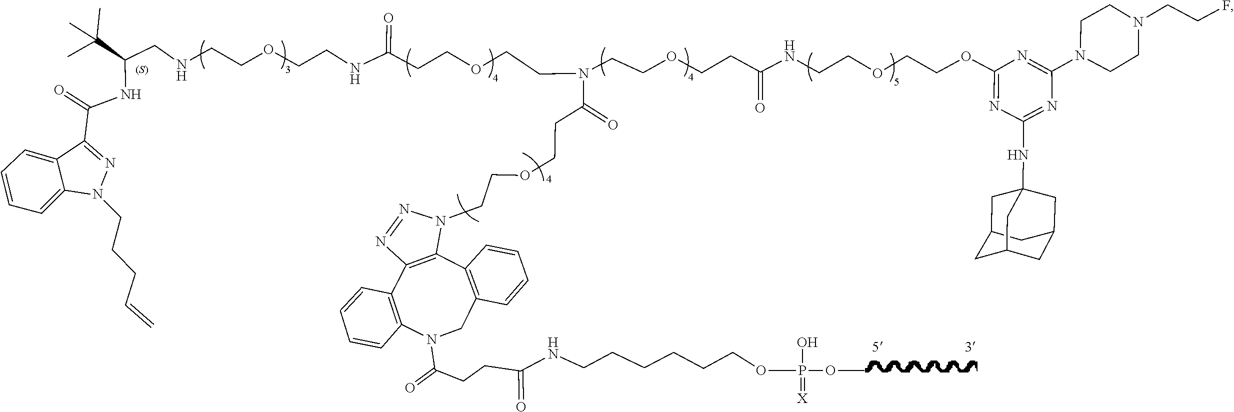

wherein:
[symbol] is an oligonucleotide; and
X is O or S.

In some embodiments, X is S. In some embodiments, X is O.

In some embodiments, any of the compounds provided herein as provided as a salt, and the salt is a potassium salt or a sodium salt.

In another aspect, the present disclosure provides compositions comprising any of the compounds, or salts thereof, and a pharmaceutically acceptable excipient.

In another aspect, the present disclosure provides methods for delivering a therapeutic oligonucleotide to the brain of a subject, comprising administration of any of the compounds provided herein, or a salt thereof, or any of the compositions provided herein, to the subject. In some embodiments, the therapeutic oligonucleotide is delivered to one or more brain regions selected from the group consisting of the striatum, the cerebellum, the brain stem, the hippocampus, the frontal cortex, and the spinal cord.

In another aspect, the present disclosure provides methods for treating or ameliorating a disease, disorder, or symptom thereof in a subject, comprising administration of any of the compounds provided herein, or a salt thereof, or any of the compositions provided herein, to the subject. In some embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In some embodiments, the administration is intrathecal administration or intracerebroventricular (ICV) administration.

In another aspect, the present disclosure provides precursor compounds, and stereoisomers, tautomers, and salts thereof, of any one of structural Formulae (A)-(M):

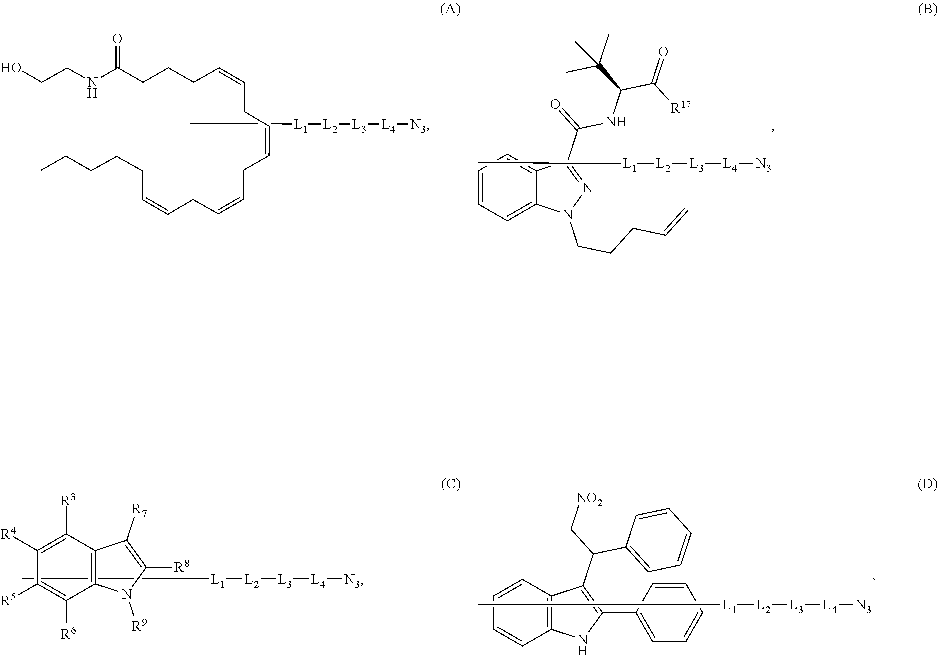

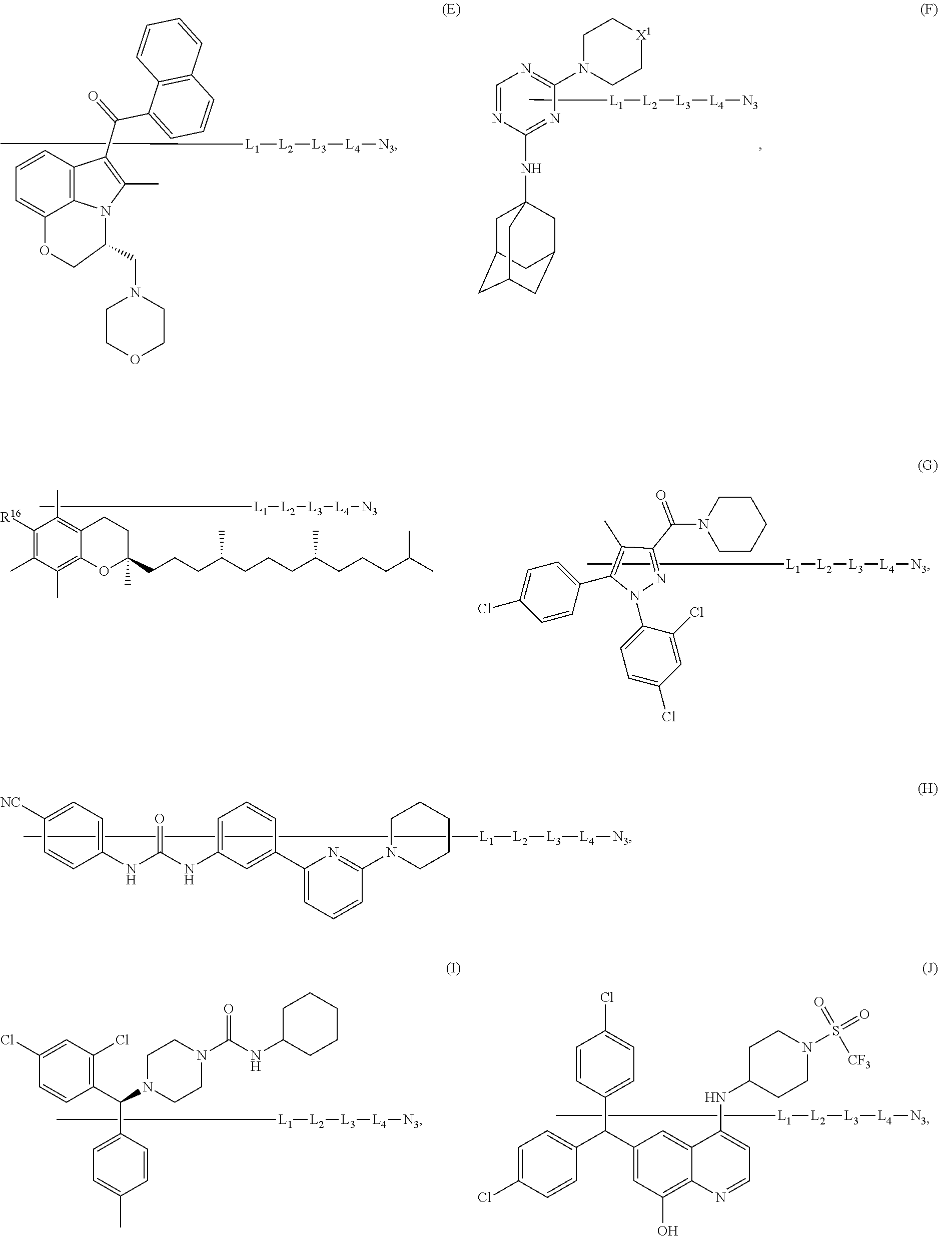

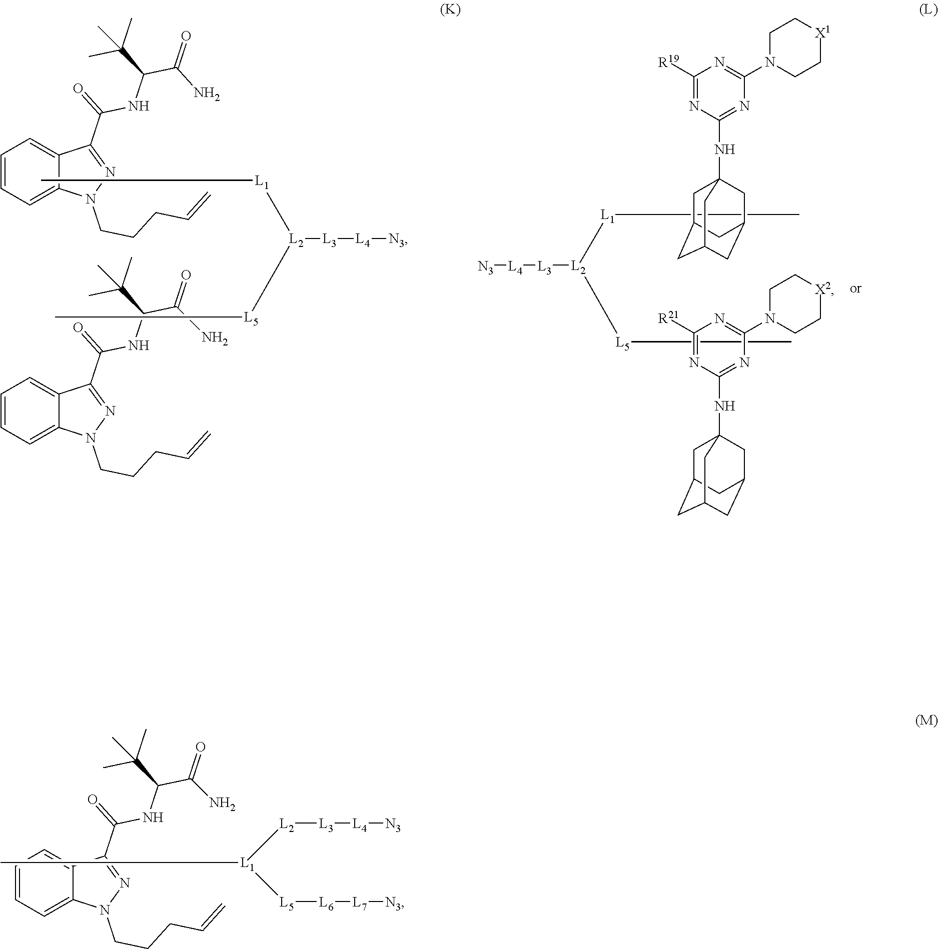

wherein:
each of L¹, L², L³, L⁴, L⁵, L⁶, and L⁷ is independently a linker, bond, or absent;
X¹ is NR¹⁰ or CR¹¹R¹²;
X² is NR¹³ or CR¹⁴R¹⁵;
R³, R⁴, R⁵, R⁶, and R⁸ are each independently hydrogen, halogen, optionally substituted alkyl, optionally substituted heteroalkyl, optionally substituted cycloalkyl, optionally substituted heterocycloalkyl, optionally substituted aryl, or optionally substituted heteroaryl;
R⁹ is hydrogen, optionally substituted alkyl, or optionally substituted heteroalkyl; or R⁶ and R⁹ substituents may be joined together form an optionally substituted heterocycloalkyl or optionally substituted heteroaryl;
R⁷ is hydrogen, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D};
—NR^{7B}C(O)OR^{7D}, —NR^{7B}OR^{7D}, optionally substituted alkyl, optionally substituted heteroalkyl, optionally substituted cycloalkyl, optionally substituted heterocycloalkyl, optionally substituted aryl, or optionally substituted heteroaryl;
R^{7A}, R^{7B}, R^{7C}, R^{7D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{7B} and R^{7C} substituents bonded to the same nitrogen atom may optionally be joined to form a substituted or unsubstituted heterocycloalkyl or substituted or unsubstituted heteroaryl;
n7 is 0, 1, 2, 3, or 4;
v7 is 1 or 2;
R¹⁰, R¹¹, R¹², R¹, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, —CN, —N₃, —NO₂, —NR^{16B}R^{16C}, —C(O)R^{16D}, —C(O)OR^{16D}, —C(O)NR^{16B}R^{16C}, —OR^{16A}, —NR^{16B}C(O)R^{16D}, optionally substituted alkyl, optionally substituted heteroalkyl, optionally substituted cycloalkyl, optionally substituted heterocycloalkyl, optionally substituted aryl, or optionally substituted heteroaryl;
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R^{18C}, —NHNR^{18B}R^{18C}, —ONR^{18B}R^{18C}, —NHC(O)NHNR^{18B}R^{18C}, —NHC(O)NR^{18B}R^{18C}, —NR^{18B}R^{18C}, —C(O)R^{18D}, —C(O)OR^{18D}, —C(O)NR^{18B}R^{18C}, —OR^{18A}, —NR^{18B}SO₂R^{18A}, —NR^{18B}C(O)R^{18D};
—NR^{18B}C(O)OR^{1D}, —NR^{18B}OR^{18D}, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D}, —NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R²⁰ is hydrogen, —SOₙ₂₀R^{20A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —NR^{20B}R^{20C}, —C(O)R^{20D}, —C(O)OR^{20D}, —C(O)NR^{20B}R^{18C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is independently hydrogen, —SOₙ₂₁R^{21A}, —SOᵥ₂₁NR^{21B}R^{21C}, NHNR^{21B}R^{21C}, —ONR^{21B}R^{21C}, —NHC(O)NHNR^{21B}R^{21C}, —NHC(O)NR^{21B}R^{21C}, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl; n19 and n21 are each independently 0, 1, 2, 3, or 4;
v19 and v21 are each independently 1 or 2;
R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, R^{19D}, R^{20A}, R^{20B}, R^{20C}, R^{20D}, R^{21A}, R^{21B}, R^{21C}, and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{16B} and R^{16C}; R^{17B} and R^{17D}; R^{18B} and R^{18D}; R^{19B} and R^{19C}; R^{20B} and R^{20C}; and R^{21B} and R^{21C} substituents bonded to the same nitrogen atom may optionally be joined to form a substituted or unsubstituted heterocycloalkyl or substituted or unsubstituted heteroaryl;
n16, n17, n18, n19, n20, and n21 are each independently 0, 1, 2, 3, or 4; and
v16, v17, v18, v19, v20, and v21 are each independently 1 or 2.

In another aspect, the present disclosure provides methods for making any of the compounds provided herein, or a stereoisomer, tautomer, prodrug, or salt thereof, comprising contacting any of the precursor compounds provided herein with a compound of structural Formula (W) and/or (Q):

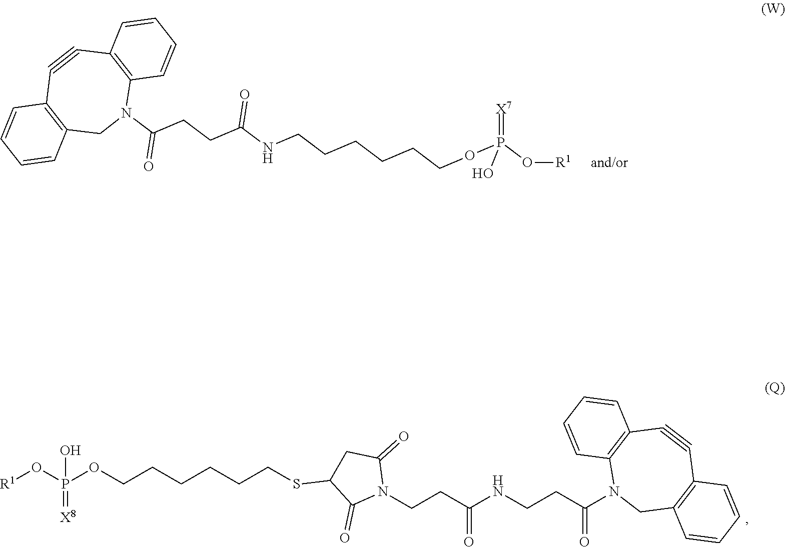

a salt thereof,
wherein X⁷ and X⁸ are each independently O or S.

In another aspect, the present disclosure provides methods for making any of the compounds provided herein, comprising one or more compounds and chemical transformations described herein, including Examples 2-24.

### BRIEF DESCRIPTION OF THE DRAWINGS

FIG. 1 provides a ¹H NMR of compound 2.

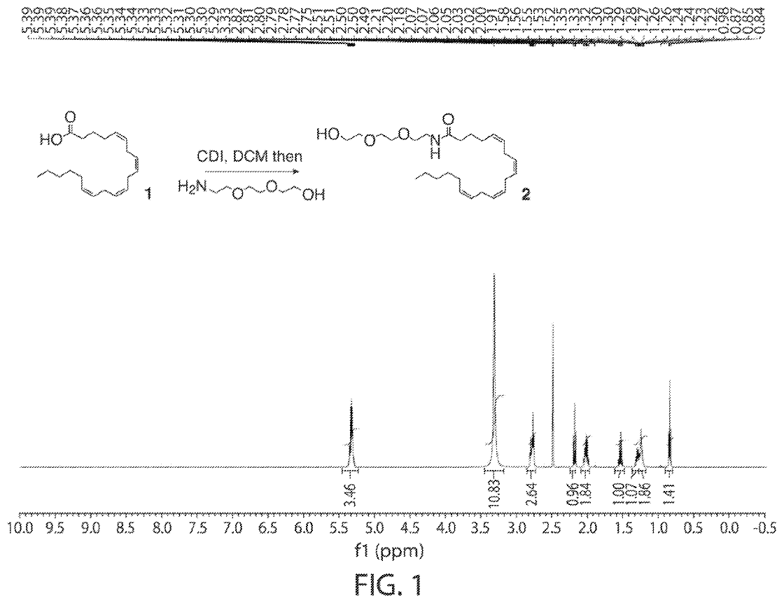

FIG. 2 provides a ¹H NMR of compound 3.

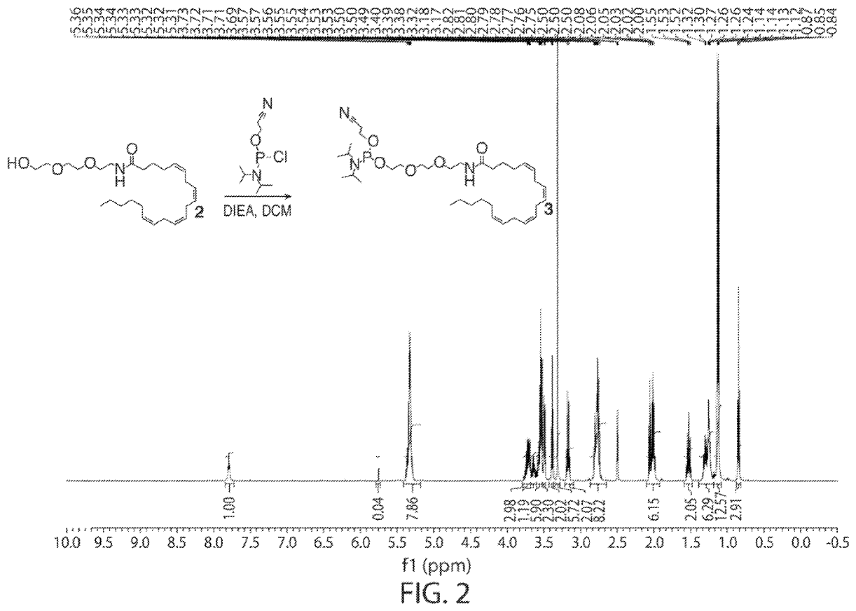

FIG. 3 provides a ³¹P NMR of compound 3.

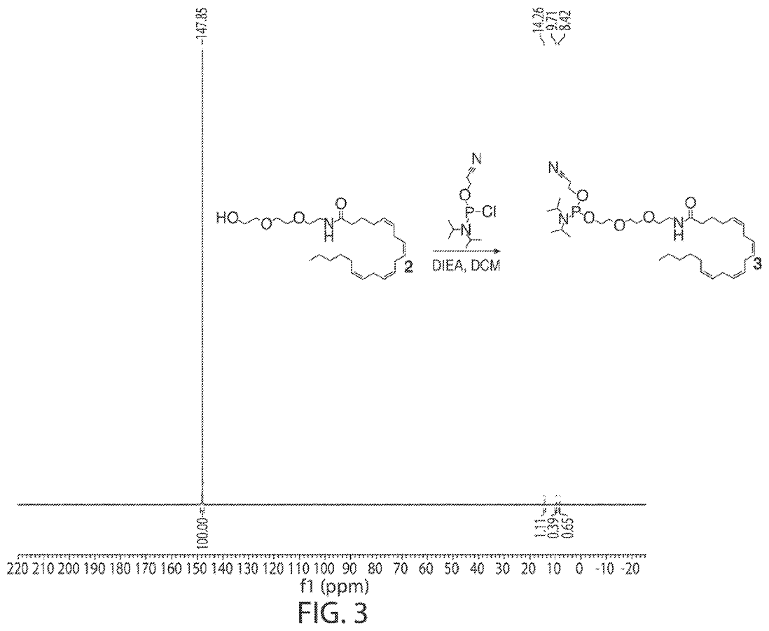

FIG. 4 provides a ¹H NMR of compound 4.

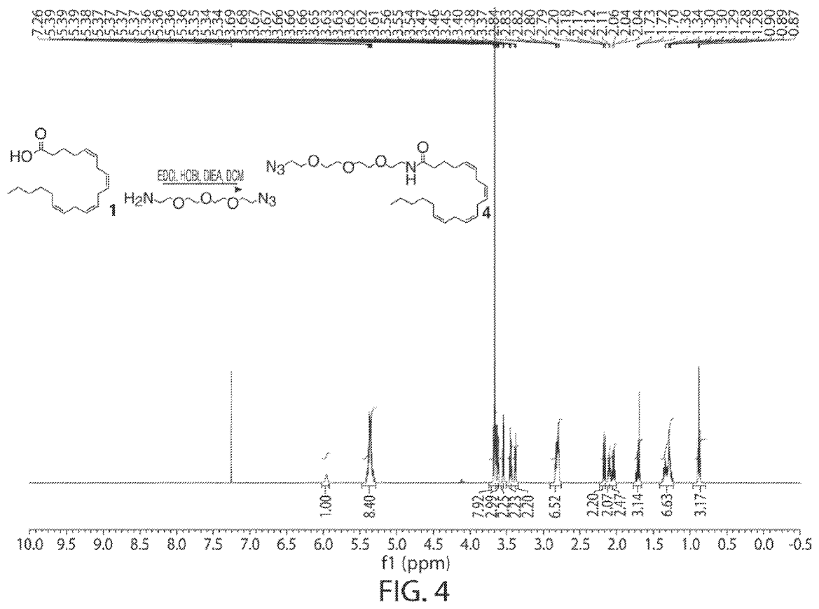

FIG. 5 provides a ¹H NMR of compound 6.

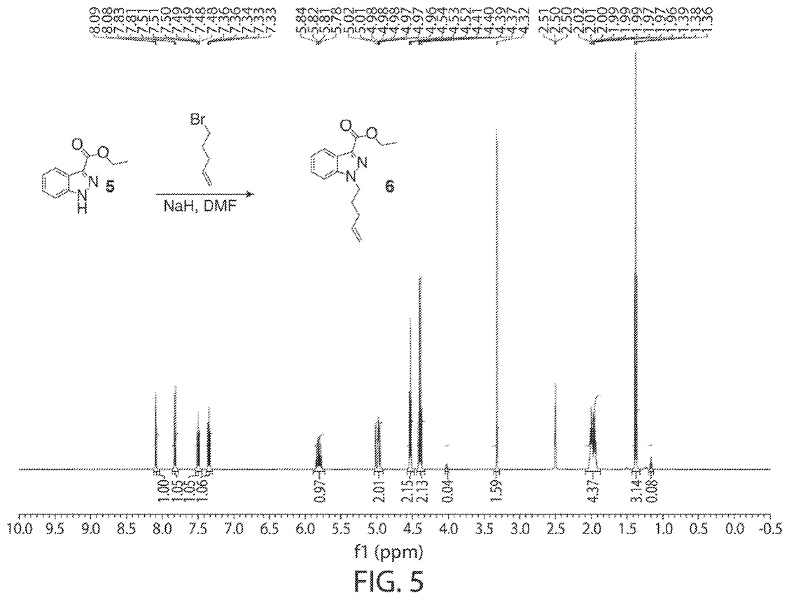

FIG. 6 provides a ¹H NMR of compound 10.

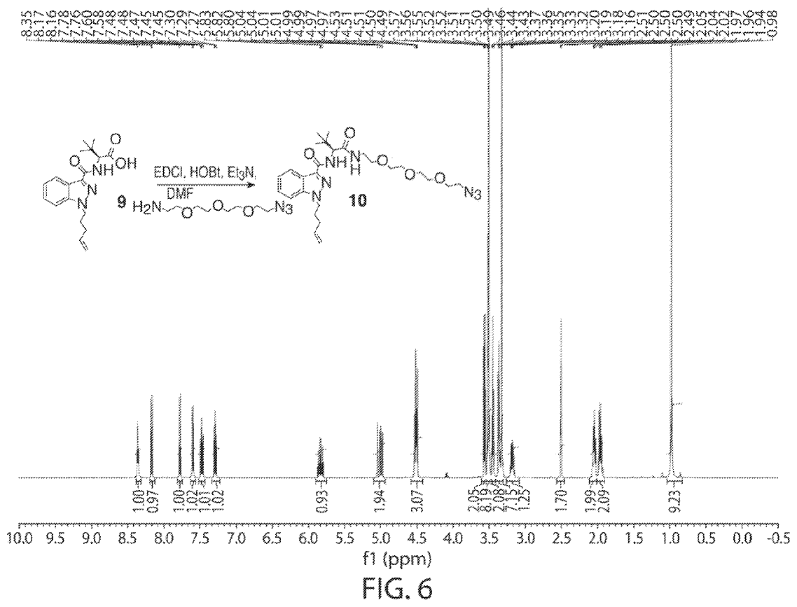

FIG. 7 provides a ¹H NMR of compound 11.

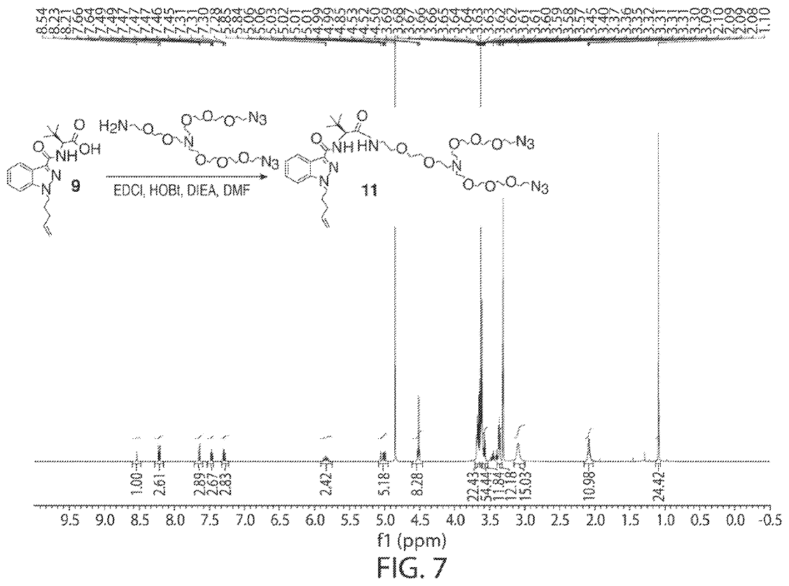

FIG. 8 provides a ¹H NMR of compound 15.

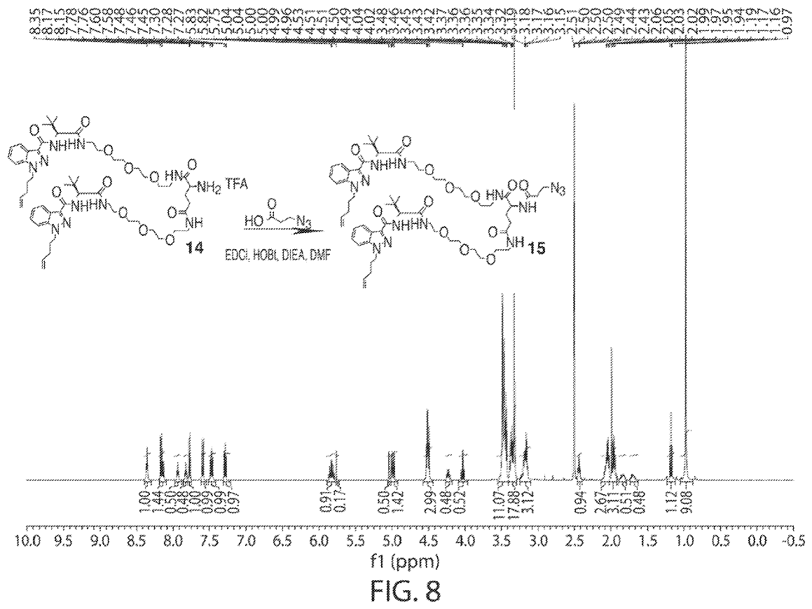

FIG. 9 provides a mass spectrum of compound 15.

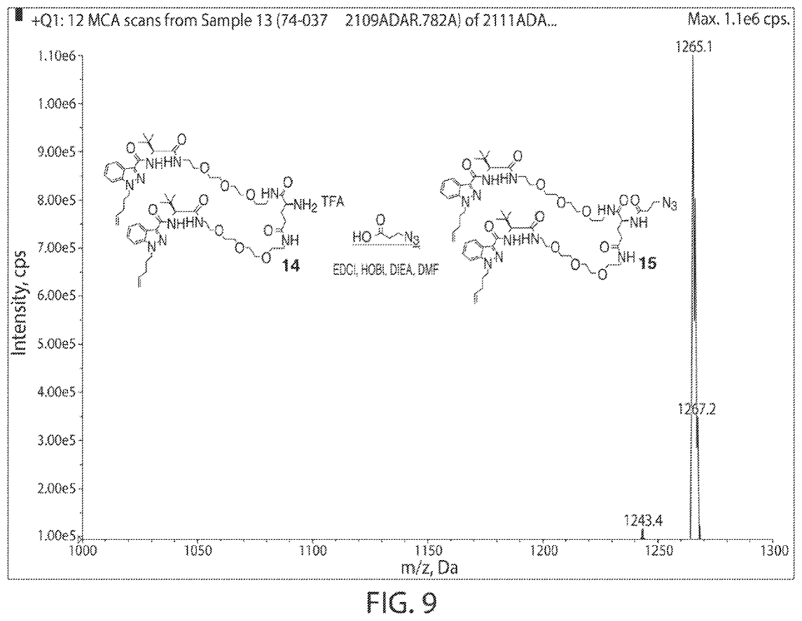

### DETAILED DESCRIPTION

### Definitions

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included,” is not limiting. The section headings used herein are for organizational purposes only and are not to be construed as limiting the subject matter described.

Unless otherwise indicated, the following terms have the following meanings:

As used herein, the term “treating” a disorder encompasses ameliorating, mitigating and/or managing the disorder and/or conditions that may cause the disorder. The terms “treating” and “treatment” refer to a method of alleviating or abating a disease and/or its attendant symptoms. In accordance with the present disclosure, “treating” includes blocking, inhibiting, attenuating, protecting against, modulating, reversing the effects of, and reducing the occurrence of, e.g., the harmful effects of a disorder. As used herein, “inhibiting” encompasses preventing, reducing, and halting progression.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high-performance liquid chromatography (HPLC). Particularly, in certain embodiments, the compound is at least 85% pure, more preferably at least 90% pure, more preferably at least 95% pure, and most preferably at least 99% pure.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal, and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any non-tolerable or detrimental effects (e.g., side effects) of the compound are outweighed by the therapeutically beneficial effects.

The phrases “systemic administration,” “administered systemically,” “peripheral administration” and “administered peripherally” as used herein mean the administration of a compound(s), oligonucleotide(s), drug, or other material, such that it enters the patient's circulatory system and, thus, is subject to metabolism and other like processes.

The term “therapeutically effective amount” refers to the amount of the compound being administered sufficient to prevent development of or alleviate to some extent one or more of the symptoms of the condition or disorder being treated.

A therapeutically effective amount of compound (i.e., an effective dosage) may range from about 0.005 g/kg to about 200 mg/kg, preferably about 0.01 mg/kg to about 200 mg/kg, and more preferably about 0.015 mg/kg to about 30 mg/kg of body weight. In other embodiments, the therapeutically effect amount may range from about 1.0 pM to about 10 μM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 μg/kg to about 200 mg/kg of body weight, daily, weekly, monthly, quarterly, or yearly. In another example, a subject may be treated daily, weekly, monthly, quarterly, or yearly for several years in the setting of a chronic condition or illness. It will also be appreciated that the effective dosage of a compound used for treatment may increase or decrease over the course of a particular treatment.

The term “chiral” refers to molecules that have the property of non-superimposability of the mirror image partner, while the term “achiral” refers to molecules that are superimposable on their mirror image partner.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)-for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium, and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure (i.e., the R and S configurations for each asymmetric center). Therefore, single stereochemical isomers as well as enantiomeric and diastereomeric mixtures of the present compounds are within the scope of the disclosure.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

The term “diastereomers” refers to stereoisomers with two or more centers of dissymmetry and whose molecules are not mirror images of one another.

The term “enantiomers” refers to two stereoisomers of a compound that are non-superimposable mirror images of one another. An equimolar mixture of two enantiomers is called a “racemic mixture” or a “racemate.”

The term “isomers” or “stereoisomers” refers to compounds that have identical chemical constitution but differ with regard to the arrangement of the atoms or groups in space.

The term “prodrug” is meant to indicate a compound that may be converted under physiological conditions or by solvolysis to a biologically active form of the compound (e.g., biologically active form of a nucleic acid) or analogue thereof as described herein. Thus, the term “prodrug” refers to a precursor of a biologically active compound (e.g., nucleic acid) or analogue thereof that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino, or free mercapto group, respectively. Examples of suitable prodrugs include, but are not limited to, glutathione, acyloxy, thioacyloxy, 2-carboalkoxyethyl, disulfide, thiaminal, and enol ester derivatives of a phosphorus atom-modified nucleic acid. The term “pro-oligonucleotide” or “pronucleotide” or “nucleic acid prodrug” refers to an oligonucleotide which has been modified to be a prodrug of the oligonucleotide. Phosphonate and phosphate prodrugs can be found, for example, in Wiener et al., “Prodrugs or phosphonates and phosphates: crossing the membrane” Top. Curr. Chem. 2015, 360:115-160, the entirety of which is herein incorporated by reference. Prodrugs that are converted to active forms through other mechanisms in vivo are also included. In aspects, the compounds of the present disclosure are prodrugs of any of the formulae herein.

The term “prodrug” includes compounds with moieties that can be metabolized in vivo. Generally, the prodrugs are metabolized in vivo by esterases or by other mechanisms to active drugs. Examples of prodrugs and their uses are well known in the art (see, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branched or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs that are converted to active forms through other mechanisms in vivo are also included. In aspects, the compounds of the present disclosure are prodrugs of any of the formulae herein.

The term “subject” refers to animals such as mammals, including, but not limited to, primates (e.g., humans), cows, sheep, goats, horses, dogs, cats, rabbits, rats, mice, and the like. In certain embodiments, the subject is a human.

The terms “a,” “an,” and “the” refer to “one or more” when used in this application, including the claims. Thus, for example, reference to “a sample” includes a plurality of samples, unless the context clearly is to the contrary (e.g., a plurality of samples), and so forth.

Throughout this specification and the claims, the words “comprise,” “comprises,” and “comprising” are used in a non-exclusive sense, except where the context requires otherwise.

As used herein, the term “about,” when referring to a value, is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

As used herein, the term “alkyl,” by itself or as part of another substituent, means, unless otherwise stated, a straight-chained (i.e., unbranched) or branched carbon chain (or carbon), or combination thereof, which may be fully saturated, mono-, (e.g., alkene or alkenyl) or polyunsaturated (e.g., alkyne or alkynyl) and can include mono-, di-, and multivalent radicals, having the number of carbon atoms designated. For example, C₁-C₂₄ means 1 to 24 carbon atoms. A specified number of carbon atoms within this range includes, for example, C₁-C₂₀ alkyl (having 1-20 carbon atoms), C₁-C₁₂ alkyl (having 1-12 carbon atoms) and C₁-C₄ alkyl (having 1-4 carbon atoms).

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The term “lower alkyl” refers to a C₁-C₆ alkyl chain. Examples of alkyl groups include methyl, ethyl, n-propyl, isopropyl, tert-butyl, and n-pentyl. Alkyl groups may be optionally substituted with one or more substituents.

The term “haloalkyl” refers to an alkyl group that is substituted by one or more halo substituents. Examples of haloalkyl groups include fluoromethyl, difluoromethyl, trifluoromethyl, bromomethyl, chloromethyl, and 2,2,2-trifluoroethyl.

The term “arylalkenyl” refers to an unsaturated hydrocarbon chain that may be a straight chain or branched chain, containing 2 to 12 carbon atoms and at least one carbon-carbon double bond wherein one or more of the sp²-hybridized carbons of the alkenyl unit attach to an aryl moiety. Alkenyl groups may be optionally substituted with one or more substituents.

The term “arylalkynyl” refers to an unsaturated hydrocarbon chain that may be a straight chain or branched chain, containing 2 to 12 carbon atoms and at least one carbon-carbon triple bond wherein one or more of the sp-hybridized carbons of the alkynyl unit attaches to an aryl moiety. Alkynyl groups may be optionally substituted with one or more substituents.

The sp²- or sp-hybridized carbons of an alkenyl group and an alkynyl group, respectively, may optionally be the point of attachment of the alkenyl or alkynyl groups.

The term “alkoxy” refers to an —O-alkyl substituent.

As used herein, the terms “halogen,” “hal,” or “halo” mean —F, —Cl, —Br or —I.

The term “alkylthio” refers to an —S-alkyl substituent.

The term “alkoxyalkyl” refers to an -alkyl-O-alkyl substituent.

The term “haloalkoxy” refers to an —O-alkyl that is substituted by one or more halo substituents. Examples of haloalkoxy groups include trifluoromethoxy, and 2,2,2-trifluoroethoxy.

The term “haloalkoxyalkyl” refers to an -alkyl-O-alkyl′ where the alkyl′ is substituted by one or more halo substituents.

The term “haloalkylaminocarbonyl” refers to a —C(O)-amino-alkyl where the alkyl is substituted by one or more halo substituents.

The term “haloalkylthio” refers to an —S-alkyl that is substituted by one or more halo substituents. Examples of haloalkylthio groups include trifluoromethylthio, and 2,2,2-trifluoroethylthio.

The term “haloalkylcarbonyl” refers to an —C(O)-alkyl that is substituted by one or more halo substituents. An example of a haloalkylcarbonyl group includes trifluoroacetyl.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “cycloalkoxy” refers to an —O-cycloalkyl substituent.

The term “cycloalkoxyalkyl” refers to an -alkyl-O-cycloalkyl substituent.

The term “cycloalkylalkoxy” refers to an —O-alkyl-cycloalkyl substituent.

The term “cycloalkylaminocarbonyl” refers to an —C(O)—NH-cycloalkyl substituent.

The term “aryl” refers to a hydrocarbon monocyclic, bicyclic, or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “aryloxy” refers to an —O-aryl substituent.

The term “arylalkoxy” refers to an —O-alkyl-aryl substituent.

The term “arylalkylthio” refers to an —S-alkyl-aryl substituent.

The term “arylthioalkyl” refers to an -alkyl-S-aryl substituent.

The term “arylalkylaminocarbonyl” refers to a —C(O)-amino-alkyl-aryl substituent.

The term “arylalkylsulfonyl” refers to an —S(O)₂-alkyl-aryl substituent.

The term “arylalkylsulfinyl” refers to an —S(O)-alkyl-aryl substituent.

The term “aryloxyalkyl” refers to an -alkyl-O-aryl substituent.

The term “alkylaryl” refers to an -aryl-alkyl substituent.

The term “arylalkyl” refers to an -alkyl-aryl substituent.

The term “heteroalkyl” by itself or in combination with another term, means, unless otherwise stated, a stable straight or branched chain, or combinations thereof, including at least one carbon atom and at least one heteroatom (e.g., O, N, P, Si, and/or S), and wherein the nitrogen and sulfur atoms may optionally be oxidized, and the nitrogen heteroatom may optionally be quaternized. The heteroatom(s) (e.g., O, N, P, Si, and/or S) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, CH₂—CH₂—NH—CH₃, CH₂—CH₂—N(CH₃)—CH₃, CH₂—S—CH₂—CH₃, CH₂—CH₂, S(O)—CH₃, CH₂—CH₂—S(O)₂—CH₃, CH═CHO—CH₃, Si(CH₃)₃, CH₂—CH═N—OCH₃, CH—CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, B, or P). A heteroalkyl moiety may include two optionally different heteroatoms (e.g., O, N, S, Si, B, and/or P). A heteroalkyl moiety may include three optionally different heteroatoms (e.g., O, N, S, Si, B, and/or P). A heteroalkyl moiety may include four optionally different heteroatoms (e.g., O, N, S, Si, B, and/or P). A heteroalkyl moiety may include five optionally different heteroatoms (e.g., O, N, S, Si, B, and/or P). A heteroalkyl moiety may include up to 8 or more optionally different heteroatoms (e.g., O, N, S, Si, B, and/or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Heteroaryl groups may be fully unsaturated, or they may be partially unsaturated and partially saturated. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heteroarylalkyl” refers to an -alkyl-heteroaryl substituent.

The term “heteroaryloxy” refers to an —O-heteroaryl substituent.

The term “heteroarylalkoxy” refers to an —O-alkyl-heteroaryl substituent.

The term “heteroaryloxyalkyl” refers to an -alkyl-O-heteroaryl substituent.

The term “nitrogen-containing heteroaryl” refers to a heteroaryl group having 1-4 ring nitrogen heteroatoms if monocyclic, 1-6 ring nitrogen heteroatoms if bicyclic, or 1-9 ring nitrogen heteroatoms if tricyclic.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “heterocycloalkylalkyl” refers to an -alkyl-heterocycloalkyl substituent.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

The symbol “ [symbol] ” denotes the point of attachment of a chemical moiety to the remainder of a molecule or chemical formula.

The term “nucleobase” refers to nitrogen-containing biological compounds that form nucleosides. They include purine bases and pyrimidine bases. Five nucleobases—adenine (A), cytosine (C), guanine (G), thymine (T), and uracil (U)—are referred to as primary or canonical nucleobases. When a nucleobase is listed in a formula definition, it refers to that moiety covalently bonded to the recited formula.

The term “modified nucleobase” refers to derivatives of a nucleobase. Examples of modified nucleobases include, but are not limited to, xanthine, hypoxanthine, 7-methylguanine, 5,6-dihydrouracil, 5-methylcytosine, 5-hydroxymethylcytosine, purine, 2,6-diaminopurine, and 6,8-diaminopurine. When a modified nucleobase is listed in a formula definition, it refers to that moiety covalently bonded to the recited formula.

The term “substituent” and “substituent group” means an atom or group that replaces the atom or group of a named parent compound. For example, a substituent of a modified nucleoside is an atom or group that differs from the atom or group found in a naturally occurring nucleoside (e.g., a modified 2′-substituent is any atom or group at the 2′-position of a nucleoside other than H or OH). Substituent groups can be protected or unprotected. Substituents may also be further substituted with other substituent groups and may be attached directly or via a linking group such as an alkyl or hydrocarbyl group to the parent compound. Similarly, as used herein, “substituent” in reference to a chemical functional group means an atom or group of atoms that differs from the atom or group of atoms normally present in the named functional group. In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carboxamido, carbamido, carbamyl, thioureido, thiocyanato, sulfoamido, sulfonylalkyl, sulfonylaryl, mercaptoalkoxy, N-hydroxyamidinyl, or N′-aryl, N″-hydroxyamidinyl. In certain embodiments, substituents on any group include alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, thiocarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, or amido. In certain embodiments, substituents on any group include alkyl, halogen, haloalkyl, cyano, nitro, alkoxy, hydroxyl, hydroxylalkyl, carboxyl, formyl, alkylcarbonyl, alkoxycarbonyl, alkylcarbonyloxy, thio, mercapto, mercaptoalkyl, amino, aminoalkyl, dialkylamino, alkylcarbonylamino, alkylaminocarbonyl, or alkylamino.

The term “protecting group” or “protecting moiety” refers to a substituent that is commonly employed to block or protect a particular functionality while reacting other functional groups on the compound, a derivative thereof, or a conjugate thereof, and includes a nitrogen protecting group when attached to a nitrogen atom, or an oxygen protecting group when attached to an oxygen atom. Nitrogen and oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on a nitrogen atom is a nitrogen protecting group (also referred to as an amino protecting group). Nitrogen protecting groups include, but are not limited to, —OH, OR^{aa}, —N(R^{cc})₂, —C(═O)R^{aa}, —C(═O)N(R^{cc})₂, —CO₂R^{aa}, —SO₂R^{aa}, C(═NR_{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein each R^{aa}, R^{bb}, and R^{cc} is independently alkyl, cycloalkyl, aryl, or heteroaryl, each of which may be optionally substituted with 1-3 independent R^{dd}, and each R^{dd} is independently alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluencsulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pme), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)Ra, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R_{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “antisense oligonucleotide” or “antisense strand” means an oligonucleotide which includes a region that is complementary to a target nucleic acid.

The term “composition” or “pharmaceutical composition” means a mixture of substances suitable for administering to a subject. For example, a composition may comprise one or more compounds or salt thereof and a sterile aqueous solution.

The term “nucleic acid” refers to molecules composed of linked monomeric nucleotides or nucleosides. A nucleic acid includes, but is not limited to, ribonucleic acids (RNA), deoxyribonucleic acids (DNA), single-stranded nucleic acids, and double-stranded nucleic acids.

The term “nucleobase sequence” means the order of contiguous nucleobases in a nucleic acid or oligonucleotide independent of any sugar or internucleoside linkage.

The term “nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

The term “oligomeric compound” means a polymer of linked subunits. With reference to a protein, peptide, polypeptide, or antibody, “subunit” refers to an amino acid or peptide bond. With reference to an oligonucleotide, “subunit” refers to a nucleotide, nucleoside, nucleobase, or sugar, or a modified nucleotide, nucleoside, nucleobase, or sugar as provided herein.

The term “oligonucleotide” means a polymer of linked nucleosides (e.g., polynucleotide, nucleic acid, polymer of nucleotides), each of which can be modified or unmodified, independent from one another. Without limitation, an oligonucleotide may be comprised of ribonucleic acids (e.g., comprised of ribonucleosides), deoxyribonucleic acids (e.g., comprised of deoxyribonucleosides), modified nucleic acids (e.g., comprised of modified nucleobases, sugars, and/or phosphate groups), or a combination thereof. Examples of oligonucleotide compounds include single-stranded and double-stranded compounds, such as, oligonucleotides, antisense oligonucleotides, interfering RNA compounds (RNAi compounds), microRNA (miRNA) targeting oligonucleotides, miRNA mimics, occupancy-based compounds (e.g., mRNA processing or translation blocking compounds and splicing compounds) and editing compounds (e.g., ADAR recruiting molecules, ADAR targeting molecules, single-stranded guide nucleic acids or a combination thereof). RNAi compounds include double-stranded compounds (e.g., short-interfering RNA (siRNA) and double-stranded RNA (dsRNA)) and single-stranded compounds (e.g., single-stranded siRNA (ssRNA), single-stranded RNAi (ssRNAi), short hairpin RNA (shRNA), and microRNA mimics) which work at least in part through the RNA-induced silencing complex (RISC) pathway resulting in sequence specific degradation and/or sequestration of a target nucleic acid through a process known as RNA interference (RNAi). The term “RNAi compound” is meant to be equivalent to other terms used to describe nucleic acid compounds that are capable of mediating sequence-specific RNA interference, for example, interfering RNA (iRNA), iRNA agent, RNAi agent, small interfering RNA, short interfering RNA, short interfering oligonucleotide, short interfering nucleic acid, short interfering modified oligonucleotide, chemically modified siRNA, and others. Additionally, the term “RNAi” is meant to be equivalent to other terms used to describe sequence-specific RNA interference.

The term “target nucleic acid,” “target RNA,” and “nucleic acid target” all mean a nucleic acid capable of being targeted by compounds described herein.

The term “therapeutic compound” includes any pharmaceutical agent or compound that provides a therapeutic benefit to a subject. Therapeutic compounds include nucleic acids, oligomeric compounds, oligonucleotides, proteins, peptides, antibodies, small molecules, and other such agents.

“Target region” means a portion of a target nucleic acid to which one or more compounds is targeted.

“Targeting moiety” means a conjugate group that provides an enhanced affinity for a selected target, e.g., molecule, cell or cell type, compartment, e.g., a cellular or organ compartment, tissue, organ, or region of the body, as, e.g., compared to a compound absent such a moiety.

“Terminal group” means a chemical group or group of atoms that is covalently linked to a terminus of an oligonucleotide.

The term “ligand” refers to a substance that binds to or otherwise interacts with a protein, nucleic acid, or other biological molecule. In some embodiments, a ligand is a small molecule. In some embodiments, a ligand binds to a protein (e.g., a receptor). In certain embodiments, a ligand binds to a CB₁ receptor.

The term “Cannabinoid Receptor Type 1” or “CB₁” means the G protein-coupled receptor for cannabinoids. In humans, CB₁ is encoded by the CNR1 gene. CB₁ is also known as cannabinoid receptor 1.

The term “Cannabinoid Receptor Type 2” or “CB₂” means the G protein-coupled receptor for cannabinoids. In humans, CB₂ is encoded by the CNR2 gene. CB₂ is also known as cannabinoid receptor 2.

The term “Cannabinoid Receptor Type 3” or “CB₃” means the G protein-coupled receptor for cannabinoids. CB₃ is also known as cannabinoid receptor 3 and is an orphan receptor. In certain embodiments, CB₃ is also known as GPR55 (i.e., G protein-coupled receptor 55), which is a putative “type 3” cannabinoid receptor.

In certain embodiments, the compounds provided herein modulate CB₁. In certain embodiments, the compounds provided herein modulate CB₂. In certain embodiments, the compounds provided herein modulate CB₃.

The term “sense oligonucleotide” or “sense strand” means the strand of a double-stranded compound that includes a region that is substantially complementary to a region of the antisense strand of the double-stranded compound.

The terms “microRNA” and “miRNA,” as may be used interchangeably herein, refer to short (e.g., about 20 to about 24 nucleotides in length) non-coding ribonucleic acids (RNAs) that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce a stem-loop precursor miRNA (pre-miRNA) approximately 70 nucleotides in length, which is further processed in the RNAi pathway. As part of this pathway, the pre-miRNA is cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing (i.e., partial complementarity) with the miRNA and most commonly results in translational inhibition or destabilization of the target mRNA. This mechanism is most often seen through the binding of the miRNA on the 3′ untranslated region (UTR) of the target mRNA, which can decrease gene expression by either inhibiting translation (for example, by blocking the access of ribosomes for translation) or directly causing degradation of the transcript. The term (i.e., miRNA) may be used herein to refer to any form of the subject miRNA (e.g., precursor, primary, and/or mature miRNA).

The terms “small interfering RNA” “short interfering RNA” and “siRNA,” as may be used interchangeably herein, refer to RNA molecules that present as non-coding double-stranded RNA (dsRNA) molecules of about 20 to about 24 nucleotides in length and are useful in RNA interference (RNAi). siRNA are often found with phosphorylated 5′ ends and hydroxylated 3′ ends, which 3′ ends typically have a 2-nucleotide overhang beyond the 5′ end of the anti-parallel strand (e.g., complementary strand of the dsRNA molecule). siRNA can interfere with the expression of specific genes through binding of target sequences (e.g., target nucleic acid sequences) to which they are complementary and promoting (e.g., facilitating, triggering, initiating) degradation of the mRNA, thereby preventing (e.g., inhibiting, silencing, interfering with) translation. After integration and separation into the RISC complex, siRNAs base-pair (e.g., full complementarity) to their target mRNA and cleave it, thereby preventing it from being used as a translation template. As discussed herein above, also part of the RNAi pathway, a miRNA-loaded RISC complex scans cytoplasmic mRNAs for potential complementarity (e.g., partial complementarity).

The term “ADAR recruiting molecule,” as may be used herein, refers to a nucleic acid that is configured to increase the concentration of Adenosine Deaminase Acting on Ribonucleic Acid (ADAR) enzyme in a locality around the nucleic acid. In some embodiments, an increased concentration is relative to the concentration in a given locality absent the ADAR recruiting molecule. In some embodiments, an ADAR recruiting molecule comprises a double-stranded RNA duplex.

The term “ADAR targeting molecule,” as may be used herein, refers to a nucleic acid that is configured to direct an ADAR molecule to a desirable location (e.g., locality). As used herein, the term “direct” refers to increasing the concentration of ADAR in the desirable location as compared to the concentration absent the ADAR targeting molecule. In some embodiments, the ADAR targeting molecule can be configured to control the desirable location by altering the sequence and/or properties of the nucleic acid (e.g., by modifications to the nucleobase, sugar, internucleoside linkage, or other component). In some embodiments, an ADAR targeting molecule comprises an ADAR recruiting molecule and a single-stranded guide nucleic acid. In some embodiments, an ADAR targeting molecule comprises a double-stranded RNA duplex and a single-stranded guide nucleic acid.

The term “single-stranded guide nucleic acid” or “guide RNA” as may be used herein, refers to a nucleic acid of a single strand, which comprises a specific sequence that is at least partially complementary to a target sequence. In some embodiments, the target sequence is at, adjacent to, or in proximity to, a locality where it is desirable to modulate ADAR concentration. In some embodiments, the level of complementarity is sufficient to facilitate binding (e.g., annealing) of the single-stranded guide nucleic acid to the target sequence.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

The term “isotopic variant” refers to a therapeutic agent (e.g., a compound and/or modified oligonucleotide disclosed herein) that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (H), deuterium (2H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I). In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O) fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²³I), iodine-127 (¹²⁷I), iodine-129 (¹²⁹I), and iodine-131 (¹³¹I).

It will be understood that, in a therapeutic agent (e.g., a compound and/or modified oligonucleotide disclosed herein), any hydrogen can be ²H, for example, or any carbon can be ^{13C}, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

“Modified oligonucleotide” means an oligonucleotide, wherein at least one sugar, nucleobase, or internucleoside linkage is modified.

“Nucleobase sequence” means the order of contiguous nucleobases in a nucleic acid or oligonucleotide independent of any sugar or internucleoside linkage.

The term “oligomeric duplex” means a duplex formed by two oligomeric compounds having complementary nucleobase sequences. Each oligomeric compound of an oligomeric duplex may be referred to as a “duplexed oligomeric compound.” The oligonucleotides of each oligomeric compound of an oligomeric duplex may include non-complementary overhanging nucleosides. In some embodiments, the terms “duplexed oligomeric compound” and “modified oligonucleotide” are used interchangeably. In other embodiments, the terms “oligomeric duplex” and “compound” are used interchangeably.

“Phosphorothioate linkage” means a modified phosphate linkage in which one of the non-bridging oxygen atoms is replaced with a sulfur atom.

The terms “RNA interference compound,” “RNAi compound,” and/or “iRNA agent” mean a compound that acts, at least in part, through an RNA-induced silencing complex (RISC) pathway or Ago2, but not through RNase H, to modulate a target nucleic acid and/or protein encoded by a target nucleic acid. RNAi compounds include, but are not limited to double-stranded siRNA, single-stranded siRNA, and microRNA, including microRNA mimics.

### Certain Embodiments

In certain embodiment, a compound comprises a CB₁ ligand and one or more linker moieties. In certain embodiments, the compound is selected from any of the formulae provided herein (e.g., Formulae (I), (I′), (I″), (I″-a), (I″-a-1), (I″-a-2), (I″-b), (I″-b-1), (I″-b-2), (I″-c), (I″-c-1), (I″-c-2), (II), (II′), (II-a), (III), (III-a), (VIII), (VIII-a), (VIII-a-1), (VIII-a-2), (VIII-b), (VIII-b-1), (VIII-c), (VIII-c-1), (VIII-c-2), (VIII-d), (VIII-d-1), (VIII-d-2), (IX), (IX-a-1), (IX-a-2), (X), (X-a), (X-a-1), (IV′), (IV″), (IV″-a), (IV″-b), (IV″-b-1), (IV), (V), (V-a), (IX), (IX-a), (XI), (XI-a), (VI), (VII), (VII-a), (XII), (XII-a), (XII-a-1), (XII-b), and (XII-c)), or salt thereof as described herein. In certain embodiments, the one or more linker moieties (L₁, L₂, L₃, L₄, L₅, L₆, L₇, etc.) links the CB₁ ligand to a therapeutic, prophylactic, or diagnostic agent. In certain embodiments, the compound further comprises one or more therapeutic, prophylactic, or diagnostic agents. In certain embodiments, a therapeutic, prophylactic, or diagnostic agent is a small molecule, or oligomeric compound. In certain embodiments, the oligomeric compound comprises a protein, peptide, antibody, oligonucleotide, or combination thereof.

In certain embodiments, an oligomeric compound is any of those described herein. In certain embodiments, the oligomeric compound is about 10-50 subunits in length. In certain embodiments the oligomeric compound is an oligonucleotide. In certain embodiments, an oligonucleotide is any of those described herein. In certain embodiments, the oligonucleotide is 8 to 80 linked nucleosides in length, 12-50 linked nucleosides in length, 12-30 linked nucleosides in length, or 15-30 linked nucleosides in length.

In certain embodiments, the oligonucleotide is a modified oligonucleotide comprising at least one modified internucleoside linkage, at least one modified sugar, or at least one modified nucleobase.

In certain embodiments, the oligonucleotide is single stranded. In certain embodiments, the oligonucleotide is double-stranded. In certain embodiments, the oligonucleotide is double-stranded over a portion of its length. In certain embodiments, the oligonucleotide comprises ribonucleic acids (e.g., comprised of ribonucleosides), deoxyribonucleic acids (e.g., comprised of deoxyribonucleosides), or a combination thereof. In certain embodiments, the oligonucleotide is a small interfering RNA (siRNA), a microRNA (miRNA) antagonist, an miRNA mimic, an ADAR recruiting molecule, an ADAR targeting molecule, a guide RNA, an antisense oligonucleotide, a short hairpin RNA (shRNA), or combinations thereof.

In some embodiments, a linker is a bond. In some embodiments, a linker is an optionally substituted PEG linker. In some embodiments, a linker is three or four PEG units in length. In certain embodiments, a linker comprises the structure

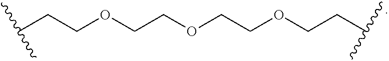 [SMILES: *CCOCCOCCOCC*]

In some embodiments, a linker is two or three PEG units in length.

In some embodiments, a linker is an optionally substituted heteroaryl linker. In some embodiments, a linker is an optionally substituted partially unsaturated heteroaryl linker. In some embodiments, a linker comprises the structure

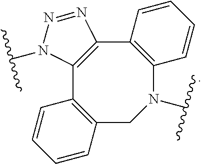 [SMILES: *N1Cc2ccccc2-c2c(nnn2*)-c2ccccc21]

In some embodiments, a linker is an optionally substituted heteroalkyl linker. In some embodiments, a linker is substituted with one or more ═O substituents. In certain embodiments, a linker comprises the structure

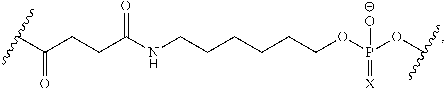

wherein X is O or S.

In some embodiments, a linker comprises the structure

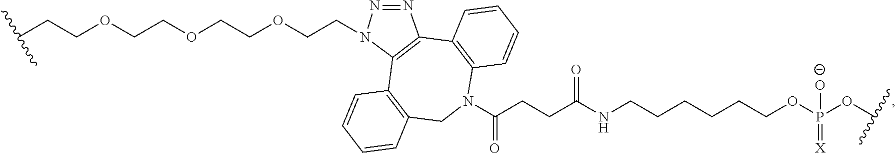

wherein X is O or S.

In some embodiments, a linker is a phosphodiester bond or a phosphorothioate bond. In certain embodiments, a linker comprises the structure

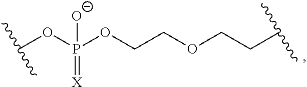

wherein X is O or S.

In certain embodiments, a linker comprises the structure

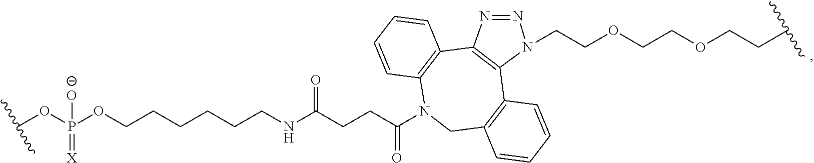

wherein X is O or S.

In some embodiments, a linker is an optionally substituted PEG linker. In some embodiments, a linker is an optionally substituted PEG linker three PEG units in length. In some embodiments, a linker is an optionally substituted heteroalkyl linker. In some embodiments, the heteroalkyl linker is substituted with one or more ═O substituents. In certain embodiments, a linker comprises the structure

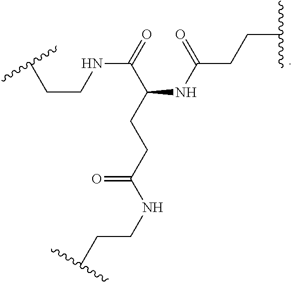 [SMILES: *CCNC(=O)CC[C@H](NC(=O)CC*)C(=O)NCC*]

In some embodiments, a linker is an optionally substituted heteroaryl linker. In some embodiments, a linker is an optionally substituted partially unsaturated heteroaryl linker. In certain embodiments, a linker comprises the structure

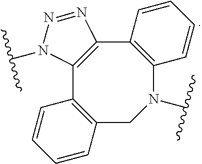 [SMILES: *N1Cc2ccccc2-c2c(nnn2*)-c2ccccc21]

In some embodiments, a linker is an optionally substituted heteroalkyl linker. In some embodiments, the heteroalkyl linker is substituted with one or more ═O substituents. In certain embodiments, a linker comprises the structure

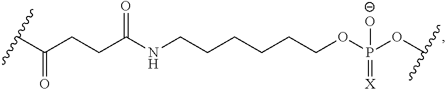

wherein X is O or S. In some embodiments, a linker comprises the structure

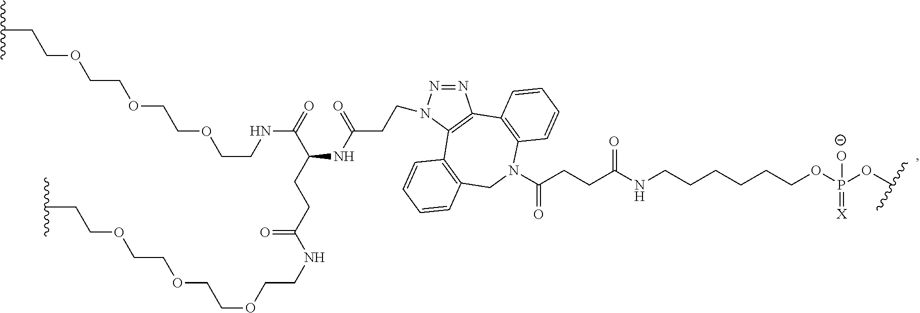

wherein X is O or S.

In some embodiments, a linker is an optionally substituted PEG linker. In certain embodiments, a linker is an optionally substituted PEG linker two or three PEG units in length. In some embodiments, a linker is an optionally substituted PEG linker. In some embodiments, a linker is an optionally substituted PEG linker three or four PEG units in length. In certain embodiments, a linker comprises the structure

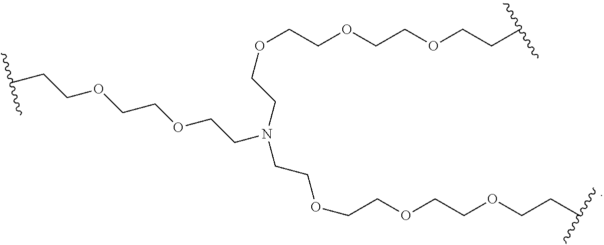 [SMILES: *CCOCCOCCOCCN(CCOCCOCC*)CCOCCOCCOCC*]

In some embodiments, a linker is an optionally substituted heteroaryl linker. In some embodiments, a linker is an optionally substituted partially unsaturated heteroaryl linker. In certain embodiments, a linker comprises the structure

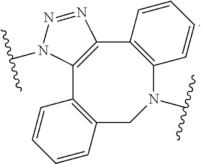 [SMILES: *N1Cc2ccccc2-c2c(nnn2*)-c2ccccc21]

In some embodiments, a linker is an optionally substituted heteroalkyl linker. In some embodiments, the heteroalkyl linker is substituted with one or more ═O substituents. In certain embodiments, a linker comprises the structure

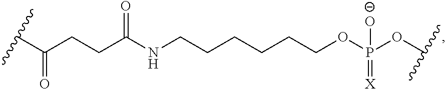

wherein X is O or S. In some embodiments, a linker comprises the structure

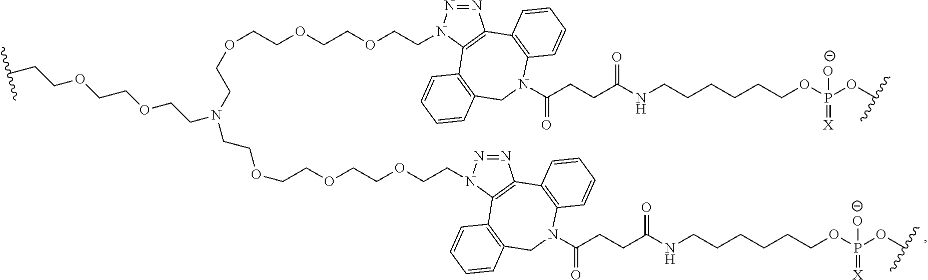

wherein X is O or S.

In certain embodiments, a compound comprises or consists of one of the structures:

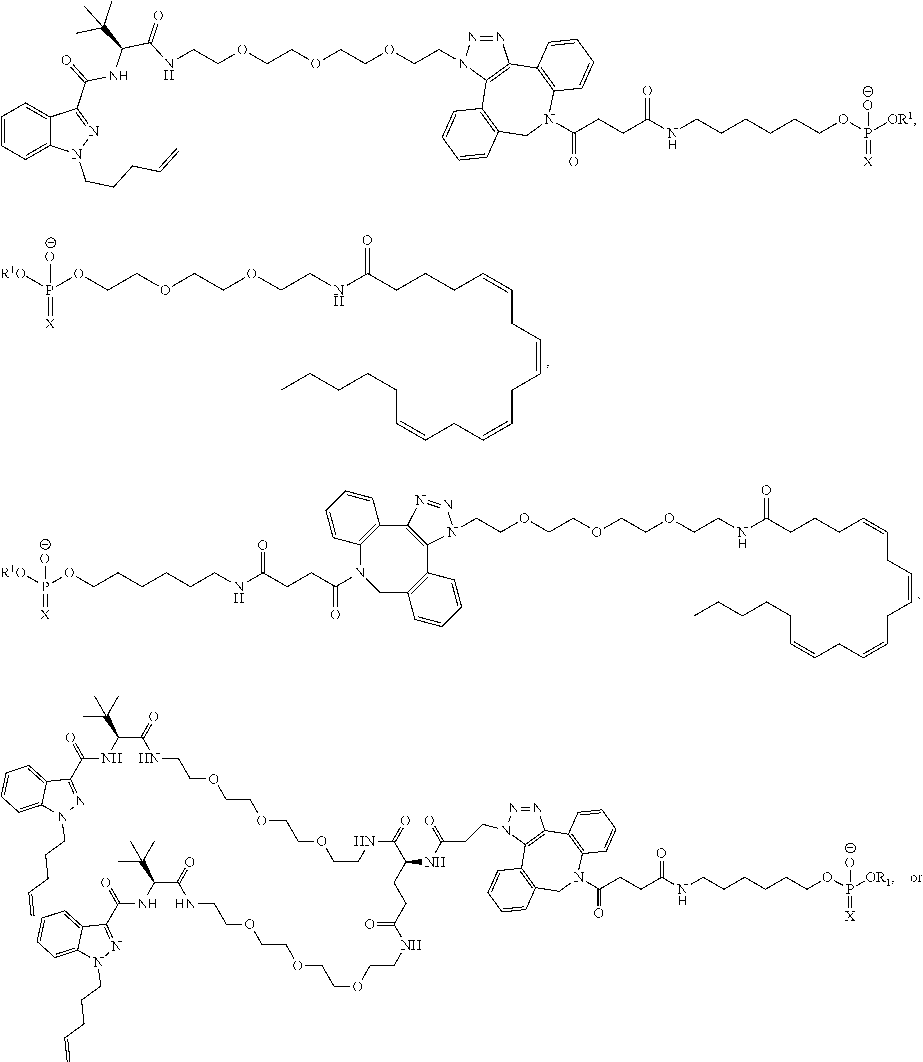

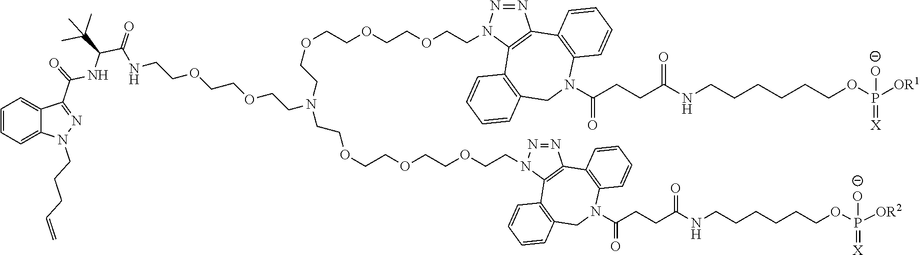

or a salt thereof, wherein X is O or S.

In some embodiments, X is O. In some embodiments, X is S.

In some embodiments, R¹ (and R² if present) comprises an oligonucleotide. In some embodiments, the oligonucleotide is attached at its 5′ end. In some embodiments, the oligonucleotide is attached at its 3′ end. In some embodiments, the oligonucleotide is attached at an internal position on the oligonucleotide. In some embodiments the internal position is at an internucleoside linkage. In some embodiments, R¹ comprises an oligonucleotide conjugated to one or more additional CB₁ ligands. In some embodiments, the oligonucleotide is conjugated to two, three, four, five, or more than five additional CB₁ ligands. In certain embodiments, the additional CB₁ ligands are conjugated to the oligonucleotide at the 5′ end of the oligonucleotide, the 3′ end of the oligonucleotide, one or more internal positions on the oligonucleotide, or any combination thereof. In certain embodiments, the oligonucleotide is a modified oligonucleotide.

Certain embodiments provide a composition comprising a compound of any embodiment herein, and a pharmaceutically acceptable carrier or excipient.

Certain embodiments provide a composition comprising a compound of any embodiment herein, for use in therapy.

In certain embodiments, a method for delivering an agent to cell comprises contacting the cell with the compound of any embodiments herein, thereby delivering the agent to the cell. In certain embodiments, the cell expresses CB₁ on the surface of the cell. In certain embodiments, the cell is a brain cell. In certain embodiments the cell is a cell of the frontal cortex. In certain embodiments, the cell is a cell of the striatum. In certain embodiments, the cell is a cell of the cerebellum. In certain embodiments, the cell is a cell of the brain stem. In certain embodiments, the cell is a cell of the hippocampus. In certain embodiments, the cell is a cell of the spinal cord. In certain embodiments, the agent is a therapeutic agent or diagnostic agent. In certain embodiments, the cell is in an animal.

In certain embodiments, a method of modulating the expression of a nucleic acid target in a cell comprises contacting the cell with the compound of any embodiments herein, thereby modulating expression of the nucleic acid target in the cell. In certain embodiments, the cell expresses CB₁ on the surface of the cell. In certain embodiments, the cell is a brain cell. In certain embodiments the cell is a cell of the frontal cortex. In certain embodiments, the cell is a cell of the striatum. In certain embodiments, the cell is a cell of the cerebellum. In certain embodiments, the cell is a cell of the brain stem. In certain embodiments, the cell is a cell of the hippocampus. In certain embodiments, the cell is a cell of the spinal cord. In certain embodiments, the agent is a therapeutic agent or diagnostic agent. In certain embodiments, contacting the cell with the compound the compound of any embodiments herein inhibits expression of the nucleic acid target. In certain embodiments, the nucleic acid target is pre-mRNA, mRNA, non-coding RNA, or miRNA. In certain embodiments, the cell is in an animal.

In certain embodiments, a method of modulating the expression of a nucleic acid target in a subject comprises administering to the subject any of the compounds or compositions provided herein, thereby modulating expression of the nucleic acid target in the subject. In certain embodiments, the expression of the nucleic acid is modulated in a cell of the subject that expresses CB₁ on the surface of the cell. In certain embodiments, the expression of the nucleic acid is modulated in a brain cell. In certain embodiments, the cell expressing CB₁ on its surface is a brain cell. In certain embodiments, the brain cell is a cell of the frontal cortex. In certain embodiments, the brain cell is a cell of the striatum. In certain embodiments, the brain cell is a cell of the cerebellum. In certain embodiments, the brain cell is a cell of the brain stem. In certain embodiments, the brain cell is a cell of the hippocampus. In certain embodiments, the brain cell is a cell of the spinal cord. In certain embodiments, the nucleic acid target is pre-mRNA, mRNA, non-coding RNA, or miRNA. In certain embodiments, the compound is administered to the subject intrathecally.

In certain embodiments, a method of treating or ameliorating a disease, disorder, or symptom thereof in a subject, comprises administering to the subject any of the compounds or compositions provided herein, thereby treating, preventing, or ameliorating a disease, disorder, or symptom in the subject. In certain embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In certain embodiments, the compound is administered to the subject intrathecally. In certain embodiments, the compound or composition is administered to the subject in a therapeutically effective amount.

In certain embodiments, a compound comprising a CB₁ ligand selectively or preferentially targets a cell expressing CB₁ compared to a cell not expressing CB₁. In certain embodiments, a compound comprising a CB₁ ligand selectively or preferentially targets a cell expressing CB₁ compared to a compound not comprising a CB₁ ligand.

Also provided herewith is the use of a compound as described herein for the manufacture of a medicament in the treatment of a disease or disorder.

In another aspect, the present disclosure provides methods for making any of the compounds provided herein, comprising one or more compounds and chemical transformations described herein, including Examples 2-24.

### Certain Compounds Comprising an Oligonucleotide

In certain embodiment, compounds described herein comprise oligonucleotides. In certain embodiments, an oligonucleotide has a nucleobase sequence that is at least partially complementary to a target nucleic acid sequence (e.g., an expressed target nucleic acid within a cell). In some embodiments, the oligonucleotide, upon delivery to a cell expressing a target nucleic acid, is able to modify the expression of the underlying gene. In some embodiments, the oligonucleotide, upon delivery to a cell expressing a target nucleic acid, is able to inhibit the expression of the underlying gene. The gene expression can be modified or inhibited in vitro or in vivo. In certain embodiments, an oligonucleotide comprises one or more ribonucleic acids (e.g., one or more ribonucleosides), deoxyribonucleic acids (e.g., one or more deoxyribonucleosides), modified nucleic acids (e.g., one or more modified nucleobases, sugars, and/or internucleoside linkages), or a combination thereof. In some embodiments, an oligonucleotide comprises a ribonucleic acid (RNA). In some embodiments, an oligonucleotide comprises a deoxyribonucleic acid (DNA). In some embodiments, an oligonucleotide comprises a modification (e.g., modified nucleobase, modified sugar, or modified internucleoside linkage).

In certain embodiments, an oligonucleotide is single-stranded. In some embodiments, a single-stranded oligonucleotide is single-stranded RNA (ssRNA), ssDNA, or a ssRNA/DNA hybrid (e.g., a single-stranded oligonucleotide comprised of both ribonucleosides (modified or unmodified) and deoxyribonucleosides (modified or unmodified))). In some embodiments, an oligonucleotide is double-stranded (e.g., comprised of two single-stranded nucleic acids). Such double-stranded oligonucleotides comprise a first oligonucleotide having a region complementary to a target nucleic acid and a second oligonucleotide having a region complementary to the first oligonucleotide. The first and second oligonucleotides can be independently modified. In certain embodiments the first oligonucleotide is linked to one or more CB₁ ligands. In certain embodiments, the second oligonucleotide is linked to one or more CB₁ ligands.

In some embodiments, an oligonucleotide is at least 2 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more) nucleotides in length. In some embodiments, an oligonucleotide is at least 5 nucleotides in length. In some embodiments, an oligonucleotide is at least 10 nucleotides in length. In some embodiments, an oligonucleotide is at least 15 nucleotides in length. In some embodiments, an oligonucleotide is at least 16 nucleotides in length. In some embodiments, an oligonucleotide is at least 17 nucleotides in length. In some embodiments, an oligonucleotide is at least 18 nucleotides in length. In some embodiments, an oligonucleotide is at least 19 nucleotides in length. In some embodiments, an oligonucleotide is at least 20 nucleotides in length. In some embodiments, an oligonucleotide is at least 21 nucleotides in length. In some embodiments, an oligonucleotide is at least 22 nucleotides in length. In some embodiments, an oligonucleotide is at least 23 nucleotides in length. In some embodiments, an oligonucleotide is at least 24 nucleotides in length. In some embodiments, an oligonucleotide is at least 25 nucleotides in length. In some embodiments, an oligonucleotide is at least 26 nucleotides in length. In some embodiments, an oligonucleotide is at least 27 nucleotides in length. In some embodiments, an oligonucleotide is at least 28 nucleotides in length. In some embodiments, an oligonucleotide is at least 29 nucleotides in length. In some embodiments, an oligonucleotide is at least 30 nucleotides in length. In some embodiments, an oligonucleotide is at least 40 nucleotides in length. In some embodiments, an oligonucleotide is at least 50 nucleotides in length. In some embodiments, an oligonucleotide is at least 60 nucleotides in length. In some embodiments, an oligonucleotide is at least 70 nucleotides in length. In some embodiments, an oligonucleotide is at least 80 nucleotides in length. In some embodiments, an oligonucleotide is at least 90 nucleotides in length. In some embodiments, an oligonucleotide is at least 100 nucleotides in length. In some embodiments, an oligonucleotide is at least 150 nucleotides in length.

In some embodiments, an oligonucleotide is less than or equal to 150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150) nucleotides in length. In some embodiments, an oligonucleotide is less than or equal to 150 nucleotides in length. In some embodiments, an oligonucleotide is less than or equal to 100 nucleotides in length. In some embodiments, an oligonucleotide is less than or equal to 90 nucleotides in length. In some embodiments, an oligonucleotide is less than or equal to 80 nucleotides in length. In some embodiments, an oligonucleotide is less than or equal to 70 nucleotides in length. In some embodiments, an oligonucleotide is less than or equal to 60 nucleotides in length. In some embodiments, an oligonucleotide is less than or equal to 50 nucleotides in length. In some embodiments, an oligonucleotide is less than or equal to 40 nucleotides in length. In some embodiments, an oligonucleotide is less than or equal to 30 nucleotides in length. In some embodiments, an oligonucleotide is less than or equal to 29 nucleotides in length. In some embodiments, an oligonucleotide is less than or equal to 28 nucleotides in length. In some embodiments, an oligonucleotide is less than or equal to 27 nucleotides in length. In some embodiments, an oligonucleotide is less than or equal to 26 nucleotides in length. In some embodiments, an oligonucleotide is less than or equal to 25 nucleotides in length. In some embodiments, an oligonucleotide is less than or equal to 24 nucleotides in length. In some embodiments, an oligonucleotide is less than or equal to 23 nucleotides in length. In some embodiments, an oligonucleotide is less than or equal to 22 nucleotides in length. In some embodiments, an oligonucleotide is less than or equal to 21 nucleotides in length. In some embodiments, an oligonucleotide is less than or equal to 20 nucleotides in length. In some embodiments, an oligonucleotide is less than or equal to 19 nucleotides in length. In some embodiments, an oligonucleotide is less than or equal to 18 nucleotides in length. In some embodiments, an oligonucleotide is less than or equal to 17 nucleotides in length. In some embodiments, an oligonucleotide is less than or equal to 16 nucleotides in length. In some embodiments, an oligonucleotide is less than or equal to 15 nucleotides in length. In some embodiments, an oligonucleotide is less than or equal to 10 nucleotides in length. In some embodiments, an oligonucleotide is less than or equal to 5 nucleotides in length.

In some embodiments, an oligonucleotide is about 5 nucleotides in length to about 150 nucleotides in length. In some embodiments, an oligonucleotide is about 10 nucleotides in length to about 100 nucleotides in length. In some embodiments, an oligonucleotide is about 20 nucleotides in length to about 90 nucleotides in length. In some embodiments, an oligonucleotide is about 30 nucleotides in length to about 80 nucleotides in length. In some embodiments, an oligonucleotide is about 40 nucleotides in length to about 70 nucleotides in length. In some embodiments, an oligonucleotide is about 50 nucleotides in length to about 60 nucleotides in length.

In some embodiments, an oligonucleotide is a therapeutic oligonucleotide. A therapeutic oligonucleotide may comprise, for example, without limitation, a small interfering RNA (siRNA), a microRNA (miRNA) antagonist, a miRNA mimic, an ADAR recruiting molecule, an ADAR targeting molecule, a guide RNA, an antisense oligonucleotide, a short hairpin RNA (shRNA), or combinations thereof.

In certain embodiments, a miRNA is a precursor, primary, and/or mature miRNA.

In certain embodiments, an oligonucleotide comprises or consists of an antisense oligonucleotide. In certain embodiments, an antisense oligonucleotide is complementary to an mRNA. In certain embodiments, an antisense oligonucleotide is complementary to a pre-mRNA. In certain embodiments, an antisense oligonucleotide blocks translation and promotes degradation of the mRNA transcript. In certain embodiments, an antisense oligonucleotide recruits RNase H and promotes degradation of the mRNA transcript. In certain embodiments, an antisense oligonucleotide targets miRNA, inhibiting the miRNA from modulating mRNA expression and promoting degradation of the miRNA.

### Certain Modifications

In certain aspects, the disclosure relates to compounds that comprise oligonucleotides. In certain embodiments, oligonucleotides may be unmodified RNA or DNA or may be modified. In certain embodiments, the oligonucleotides are modified oligonucleotides. In certain embodiments, the modified oligonucleotides comprise at least one modified sugar, modified nucleobase, or modified internucleoside linkage relative to an unmodified RNA or DNA. In certain embodiments, an oligonucleotide has a modified nucleoside. A modified nucleoside may comprise a modified sugar, a modified nucleobase, or both a modified sugar and a modified nucleobase. Modified oligonucleotides may also include end modifications, e.g., 5′-end modifications and 3′-end modifications.

### Sugar Modifications and Motifs

In certain embodiments, a modified sugar is a substituted furanosyl sugar or non-bicyclic modified sugar. In certain embodiments, a modified sugar is a bicyclic or tricyclic modified sugar. In certain embodiments, a modified sugar is a sugar surrogate. A sugar surrogate may comprise one or more substitutions described herein.

In certain embodiments, a modified sugar is a substituted furanosyl or non-bicyclic modified sugar. In certain embodiments, the furanosyl sugar is a ribosyl sugar. In certain embodiments, the furanosyl sugar comprises one or more substituent groups, including, but not limited to, substituent groups at the 2′, 3′, 4′, and 5′ positions.

In certain embodiments, substituents at the 2′ position include, but are not limited to, F and OCH₃ (“OMe”, “O-methyl” or “methoxy”). In certain embodiments, substituent groups at the 2′ position suitable for non-bicyclic modified sugars include, but are not limited to, halo, allyl, amino, azido, SH, CN, OCN, CF₃, OCF₃, F, Cl, Br, SCH₃, SOCH₃, SO₂CH₃, ONO₂, NO₂, N₃, and NH₂. In certain embodiments, substituent groups at the 2′ position include, but are not limited to, 0-(C₁-C₁₀) alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁ to C₁₀ alkyl or C₂ to C₁₀ alkenyl and alkynyl. In certain embodiments, substituent groups at the 2′ position include, but are not limited to, alkaryl, aralkyl, O-alkaryl, and O-aralkyl. In certain embodiments, these 2′ substituent groups can be further substituted with one or more substituent groups independently selected from hydroxyl, alkoxy, carboxy, benzyl, phenyl, nitro (NO₂), thiol, thioalkoxy, thioalkyl, halogen, alkyl, aryl, alkenyl, and alkynyl. In certain embodiments, substituent groups at the 2′ position include, but are not limited to, O[(CH₂)ₙO]ₘCH₃, O(CH₂)ₙOCH₃, O(CH₂)ₙCH₃, O(CH₂)ₙONH₂, O(CH₂)ₙNH₂, O(CH₂)ₙSCH₃, and O(CH₂)ₙON[(CH₂)·CH₃)]₂, where n and m are independently from 1 to about 10. In certain embodiments, substituent groups at the 2′ position include, but are not limited to, OCH₂CH₂OCH₃ (“MOE”), O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, substituent groups at the 4′ position suitable for non-bicyclic modified sugars include, but are not limited to, alkoxy (e.g., methoxy), alkyl, and those described in Manoharan et al., WO 2015/106128. In certain embodiments, substituent groups at the 5′ position suitable for non-bicyclic modified sugars include, but are not limited to, methyl (“Me”) (R or S), vinyl, and methoxy. In certain embodiments, one or more sugars comprise a 5′-vinylphosphonate modification. In certain embodiments, substituents described herein for the 2′, 4′, and 5′ position can be added to other specific positions on the sugar. In certain embodiments, such substituents may be added to the 3′ position of the sugar on the 3′ terminal nucleoside or the 5′ position of the 5′ terminal nucleoside. In certain embodiments, a non-bicyclic modified sugar may comprise more than one non-bridging sugar substituent. In certain such embodiments, non-bicyclic modified sugars substituents include, but are not limited to, 5′-Me-2′-F, 5′-Me-2′-OMe (including both R and S isomers). In certain embodiments, modified sugar substituents include those described in Migawa et al., WO 2008/101157 and Rajeev et al., US2013/0203836. In certain embodiments, substituent groups at the 5′ position suitable for non-bicyclic modified sugars include, but are not limited to, methyl (“Me” or “CH₃”) (R or S), vinyl, and methoxy. In certain embodiments, the 5′ modification is a 5′-monophosphate ((HO)₂(O)P—O-5′); 5′-diphosphate ((HO)₂(O)P—O—P(HO)(O)—O-5′); 5′-triphosphatc ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′adenosine cap (Appp), and any modified or unmodified nucleotide cap structure (N—O-5′(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′phosphorothiolate ((HO)₂(O)P—S-5′); any additional combination of oxygen/sulfur replaced monophosphate, diphosphate, and triphosphates (e.g., 5′-alpha-thiotriphosphate, 5′-gammathiotriphosphate, etc.), 5′-phosphoramidates ((HO)₂(O)P—NH-5′, (HO)(NH₂)(O)P—O-5′), 5′alkylphosphonates (R=alkyl=methyl, ethyl, isopropyl, propyl, etc., e.g., RP(OH)(O)—O-5′-, 5′alkenylphosphonates (i.e., vinyl, substituted vinyl), (OH)₂(O)P-5′-CH₂—), 5′alkyletherphosphonates (R=alkylether=methoxymethyl (MeOCH₂—), ethoxymethyl, etc., e.g., RP(OH)(O)—O-5′-). In certain embodiments, one or more sugars comprise a 5′-vinylphosphonate modification. In certain embodiments the 5′ modification is at the terminus of an oligonucleotide. In certain embodiments the 5′ modification is at the terminus of an antisense oligonucleotide.

In certain embodiments, a modified sugar is a bicyclic sugar. A bicyclic sugar is a modified sugar comprising two rings, wherein the second ring is formed via a bridge connecting two of the atoms in the first ring, thereby forming a bicyclic structure. In certain embodiments, a bicyclic sugar comprises a bridging substituent that bridges two atoms of the furanosyl ring to form a second ring. In certain embodiments, a bicyclic sugar does not comprise a furanosyl moiety. A “bicyclic nucleoside” (“BNA”) is a nucleoside having a bicyclic sugar. In certain embodiments, the bicyclic sugar comprises a bridge between the 4′ and 2′ furanose ring atoms. In certain embodiments, the bicyclic sugar comprises a bridge between the 5′ and 3′ furanose ring atoms. In certain such embodiments, the furanose ring is a ribose ring. In certain embodiments, 4′ to 2′ bridging substituents include, but are not limited to, 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (“constrained ethyl” or “cEt” when in the S configuration), 4′-CH₂O—CH₂-2′, 4′-CH₂—N(R)-2′, 4′-CH(CH₂OCH₃)—O-2′ (“constrained MOE” or “cMOE”) and analogs thereof (e.g., U.S. Pat. No. 7,399,845), 4′-C(CH₃)(CH₃)—O-2′ and analogs thereof (e.g., U.S. Pat. No. 8,278,283), 4′-CH₂—N(OCH₃)-2′ and analogs thereof (e.g., U.S. Pat. No. 8,278,425), 4′-CH₂—O—N(CH₃)-2′ (e.g., U.S. Patent Publication No. 2004/0171570), 4′-CH₂—N(R)—O-2′, wherein R is H, C₁-C₁₂ alkyl, or a protecting group (e.g., U.S. Pat. No. 7,427,672), 4′-CH₂—C(H)(CH₃)-2′ (e.g., Chattopadhyaya et al., J. Org. Chem., 2009, 74, 118-134), and 4′-CH₂—C(═CH₂)-2′ and analogs thereof (e.g., U.S. Pat. No. 8,278,426). The entire contents of each of the foregoing are hereby incorporated herein by reference. Additional representative U.S. Patents and U.S. Patent Publications that teach the preparation of bicyclic nucleic acid nucleotides include, but are not limited to, the following: U.S. Pat. Nos. 6,268,490; 6,525,191; 6,670,461; 6,770,748; 6,794,499; 6,998,484; 7,053.207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, US 2013/0190383; and WO 2013/036868, the entire contents of each of which are hereby incorporated herein by reference. Any of the foregoing bicyclic nucleosides can be prepared having one or more stereochemical sugar configurations including, for example, α-L-ribofuranose and β-D-ribofuranose (see, e.g., WO 99/14226). Specified bicyclic nucleosides herein are in the β-D configuration, unless otherwise specified.

In certain embodiments, a modified sugar is a sugar surrogate. In certain embodiments, a sugar surrogate has the oxygen atom replaced, e.g., with a sulfur, carbon or nitrogen atom. In certain such embodiments, the sugar surrogate may also comprise bridging and/or non-bridging substituents as described herein. In certain embodiments, sugar surrogates comprise rings having other than 5 atoms. In certain such embodiments, the sugar surrogate comprises a cyclobutyl moiety in place of the pentofuranosyl sugar. In certain embodiments, the sugar surrogate comprises a six membered ring in place of the pentofuranosyl sugar. In certain embodiments, the sugar surrogate comprises a tetrahydropyran (“THP”) in place of the pentofuranosyl sugar. In certain embodiments, the sugar surrogate comprises a morpholino in place of the pentofuranosyl sugar. Representative US patents that teach the preparation of such modified sugar structures include, but are not limited to, U.S. Pat. Nos. 4,981,957; 5,118,800; 5,166,315; 5,185,444; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,700,920; 7,875,733; 7,939,677, 8,088,904; 8,440,803; and 9,005,906, the entire contents of each of the foregoing are hereby incorporated herein by reference.

In some embodiments, sugar surrogates comprise acyclic moieties. In certain embodiments, the sugar surrogate is an unlocked nucleic acid (“UNA”). A UNA is unlocked acyclic nucleic acid, wherein any of the bonds of the sugar has been removed, forming an unlocked “sugar” residue. In one example, UNA also encompasses a monomer where the bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed. Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference. In certain embodiments, sugar surrogates comprise peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378, the entire contents of which is hereby incorporated herein by reference. Many other bicyclic and tricyclic sugar and sugar surrogate ring systems are known in the art that can be used in modified nucleosides.

In certain aspects, the disclosure relates to compounds comprising at least one oligonucleotide wherein the nucleosides of such oligonucleotide comprise one or more types of modified sugars and/or unmodified sugars arranged along the oligonucleotide or region thereof in a defined pattern or “sugar motif”. In certain instances, such sugar motifs include, but are not limited to, any of the patterns of sugar modifications described herein.

In certain embodiments, an oligonucleotide comprises a gapmer sugar motif. A gapmer oligonucleotide comprises or consists of a region having two external “wing” regions and a central or internal “gap” region. The gap and wing regions form a contiguous sequence of nucleosides, wherein the majority of nucleoside sugars of each of the wings differ from the majority of nucleoside sugars of the gap. In certain embodiments, the wing regions comprise a majority of modified sugars and the gap comprises a majority of unmodified sugars. In certain embodiments, the nucleosides of the gap are deoxynucleosides. Compounds with a gapmer sugar motif are described in, for example, U.S. Pat. No. 8,790,919, the entire contents of which is hereby incorporated herein by reference.

In certain embodiments, one or both oligonucleotides of a double-stranded compound comprise a triplet sugar motif. An oligonucleotide with a triplet sugar motif comprises three identical sugar modifications on three consecutive nucleosides. In certain embodiments, the triplet is at or near the cleavage site of the oligonucleotide. In certain embodiments, an oligonucleotide of a double-stranded compound may contain more than one triplet sugar motif. In certain embodiments, the identical sugar modification of the triplet sugar motif is a 2′-F modification. Compounds with a triplet sugar motif are disclosed, for example, in U.S. Pat. No. 10,668,170, the entire contents of which is incorporated herein by reference.

In certain embodiments, one or both oligonucleotides of a double-stranded compound comprise a quadruplet sugar motif. An oligonucleotide with a quadruplet sugar motif comprises four identical sugar modifications on four consecutive nucleosides. In certain embodiments, the quadruplet is at or near the cleavage site. In certain embodiments, an oligonucleotide of a double-stranded compound may contain more than one quadruplet sugar motif. In certain embodiments, the identical sugar modification of the quadruplet sugar motif is a 2′-F modification. For a double-stranded compound having a duplex region of 19-23 nucleotides in length, the cleavage site of the antisense oligonucleotide is typically around the 10, 11, and 12 positions from the 5′-end. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the sense oligonucleotide, counting from the first nucleoside of the 5′-end of the sense oligonucleotide, or, the count starting from the first paired nucleotide within the duplex region from the 5′-end of the sense oligonucleotide. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the antisense oligonucleotide, counting from the first nucleoside of the 5′-end of the antisense oligonucleotide, or, the count starting from the first paired nucleotide within the duplex region from the 5′-end of the antisense oligonucleotide. The cleavage site may change according to the length of the duplex region of the double-stranded compound and may change the position of the quadruplet accordingly.

In certain embodiments, an oligonucleotide comprises an alternating sugar motif. In certain embodiments, one or both oligonucleotides of a double-stranded compound comprise an alternating sugar motif. An oligonucleotide with an alternating sugar motif comprises at least two different sugar modifications, wherein one or more consecutive nucleosides comprising a first sugar modification alternates with one or more consecutive nucleosides comprising a second sugar modification, and one or more consecutive nucleosides comprising a third sugar modification, etc. For example, if A, B, and C each represent one type of modification to the nucleoside, the alternating motif can be “ABABABABABAB . . . ,” “AABBAABBAABB . . . ,” “AABAABAABAAB “AAABAAABAAAB . . . ,” “AAABBBAAABBB . . . ,” or “ABCABCABCABC . . . ” etc. In certain embodiments, the alternating sugar motif is repeated for at least 2, 3, 4, 5, 6, 7, 8, 9, 10, 11, 12, 13, 14, 15, 16, 17, 18, 19, 20, 21, 22, or 23 contiguous nucleobases along an oligonucleotide. In certain embodiments, the alternating sugar motif is comprised of two different sugar modifications. In certain embodiments, the alternating sugar motif comprises 2′-OMe and 2′-F sugar modifications.

In certain embodiments, each nucleoside of an oligonucleotide is independently modified with one or more sugar modifications provided herein. In certain embodiments, each oligonucleotide of a double-stranded compound independently has one or more sugar motifs provided herein. In certain embodiments, an oligonucleotide containing a sugar motif is fully modified in that each nucleoside other than the nucleosides comprising the sugar motif comprises a sugar modification.

### Nucleobase Modifications and Motifs

In certain embodiments, modified oligonucleotides comprise one or more nucleosides comprising a modified nucleobase. In certain embodiments, modified oligonucleotides comprise one or more nucleosides that do not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone.

Further nucleobases include those disclosed in U.S. Pat. No. 3,687,808; Modified Nucleosides in Biochemistry, Biotechnology and Medicine, Herdewijn, P. ed. Wiley-VCH, 2008; The Concise Encyclopedia Of Polymer Science And Engineering, pages 858-859; Kroschwitz, J. L., Ed., John Wiley & Sons, 1990, 858-859; Englisch et al., Angewandte Chemie, International Edition, 1991, 30, 613; Sanghvi, Y. S., Chapter 15, dsRNA Research and Applications, pages 289-302; Antisense Research and Applications, Crooke, S. T. and Lebleu, B., Eds., CRC Press, 1993, 273-288; Antisense Drug Technology, Crooke S. T., Ed., CRC Press, 2008, 163-166 and 442-443 (Chapters 6 and 15), each of which are hereby incorporated herein by reference.

Publications that teach the preparation of certain of the above noted modified nucleobases, as well as other modified nucleobases include without limitation, U.S. Applications 2003/0158403 and 2003/0175906; U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,005,096. 6,015,886; 6,147,200; 6,166,197; 6,166,199; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

### Internucleoside Linkage Modifications and Motifs

A 3′ to 5′ phosphodiester linkage is the naturally occurring internucleoside linkage of RNA and DNA. In certain embodiments, an oligonucleotide has one or more modified, i.e., non-naturally occurring, internucleoside linkages. Certain non-naturally occurring internucleoside linkages may impart desirable properties such as, for example, enhanced cellular uptake, enhanced affinity for target nucleic acids, and increased stability in the presence of nucleases. Representative phosphorus-containing modified internucleoside linkages include, but are not limited to, phosphotriesters, alkylphosphonates (e.g., methylphosphonates), phosphoramidates, and phosphorothioates (“P═S”), and phosphorodithioates (“HS—P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N((CH₃)—N((CH₃)—). Methods of preparation of phosphorous-containing and non-phosphorous-containing internucleoside linkages are well known to those skilled in the art. Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, an oligonucleotide comprises at least one modified internucleoside linkage. A modified internucleoside linkage may be placed at any position of an oligonucleotide. For double-stranded compounds, a modified internucleoside linkage may be placed within the sense oligonucleotide, antisense oligonucleotide, or both oligonucleotides of the double-stranded compound.

In certain embodiments, the internucleoside linkage modification may occur on every nucleoside of an oligonucleotide. In certain embodiments, internucleoside linkage modifications may occur in an alternating pattern along an oligonucleotide. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the pattern of the internucleoside linkage modification on each oligonucleotide of a double-stranded compound is the same. In certain embodiments, the pattern of the internucleoside linkage modification on each oligonucleotide of a double-stranded compound is different. In certain embodiments, a double-stranded compound comprises 6-8 modified internucleoside linkages. In certain embodiments, the 6-8 modified internucleoside linkages are phosphorothioate internucleoside linkages or alkylphosphonate internucleoside linkages. In certain embodiments, the sense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages. In certain embodiments, the antisense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages.

In certain embodiments, a double-stranded compound comprises an overhang region. In certain embodiments, a double-stranded compound comprises a phosphorothioate or alkylphosphonate internucleoside linkage modification in the overhang region. In certain embodiments, a double-stranded compound comprises a phosphorothioate or alkylphosphonate internucleotide linkage linking the overhang nucleotide with a paired nucleotide that is next to the overhang nucleotide. For instance, there may be at least two phosphorothioate internucleoside linkages between the terminal three nucleosides, in which two of the three nucleosides are overhang nucleosides, and the third is a paired nucleoside next to the overhang nucleoside. These terminal three nucleosides may be at the 3′-end of the antisense oligonucleotide, the 3′-end of the sense oligonucleotide, the 5′-end of the antisense oligonucleotide, or the 5′end of the antisense oligonucleotide.

In certain embodiments, modified oligonucleotides comprise one or more internucleoside linkages having chiral centers. Representative chiral internucleoside linkages include, but are not limited to, alkylphosphonates and phosphorothioates. Modified oligonucleotides comprising internucleoside linkages having chiral centers can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. As is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration.

### CB₁ Ligands

In some embodiments, the compounds provided herein comprise a cannabinoid receptor type 1 (CB₁, also known as cannabinoid receptor 1) ligand. In some embodiments, a CB₁ ligand is useful for directing a therapeutic, prophylactic, or diagnostic agent. In certain embodiments, a therapeutic agent is an oligonucleotide (e.g., a therapeutic oligonucleotide). In some embodiments, a CB₁ ligand directs an oligonucleotide to a locality. In some embodiments, a CB₁ ligand targets tissues. In some embodiments, the tissue is brain tissue. In some embodiments, a CB₁ ligand targets a cell receptor. In some embodiments, a cell receptor is CB₁. In some embodiments, a CB₁ receptor is in the brain. In some embodiments, a CB₁ receptor is in the frontal cortex. In some embodiments, a CB₁ receptor is in the striatum. In some embodiments, a CB₁ receptor is in the cerebellum. In some embodiments, a CB₁ receptor is in the brain stem. In some embodiments, a CB₁ receptor is in the hippocampus. In some embodiments, a CB₁ receptor is in the spinal cord.

The use of any CB₁ ligand in the compounds provided herein is contemplated by the present disclosure. CB₁ ligands are known in the art, and a person of ordinary skill in the art would be capable of identifying additional CB₁ ligands for use in the compounds described herein beyond those explicitly provided by the present disclosure. The present disclosure also contemplates the use of derivatives and prodrugs of any CB₁ ligand provided herein or known in the art in the presently described compounds, and a person of ordinary skill in the art would know how to make such derivatives and prodrugs.

In some embodiments, a CB₁ ligand is a CB₁ agonist. In some embodiments, a CB₁ ligand is a CB₁ antagonist. Exemplary CB₁ ligands for use in the present disclosure include, but are not limited to, any of the following CB₁ ligands, and derivatives thereof:

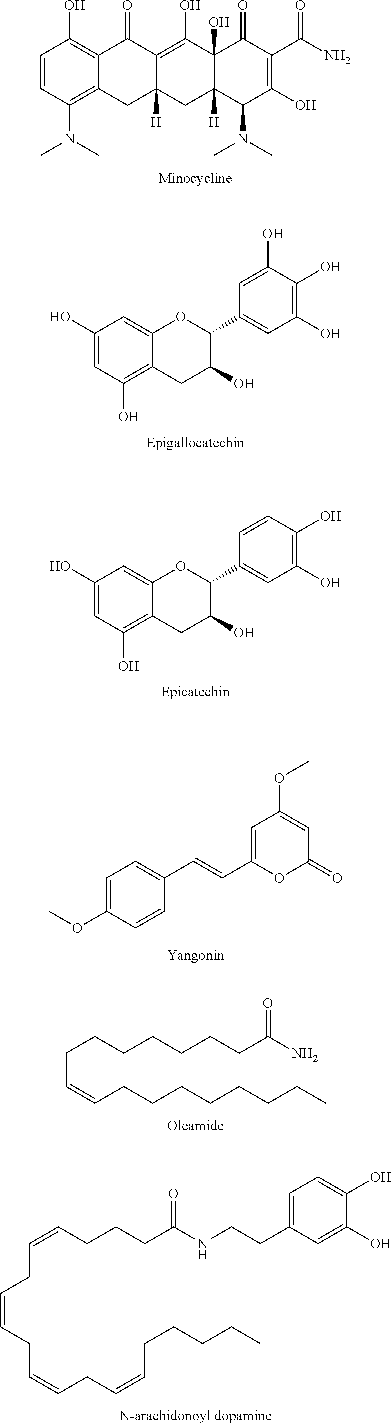 [SMILES: CN(C)c1ccc(O)c2c1C[C@H]1C[C@H]3[C@H](N(C)C)C(O)=C(C(N)=O)C(=O)[C@@]3(O)C(O)=C1C2=O ; Oc1cc(O)c2c(c1)O[C@H](c1cc(O)c(O)c(O)c1)[C@@H](O)C2 ; Oc1cc(O)c2c(c1)O[C@H](c1ccc(O)c(O)c1)[C@@H](O)C2 ; COc1ccc(/C=C/c2cc(OC)cc(=O)o2)cc1 ; CCCCCCCC/C=C\CCCCCCCC(N)=O ; CCCCC/C=C\C/C=C\C/C=C\C/C=C\CCCC(=O)NCCc1ccc(O)c(O)c1]

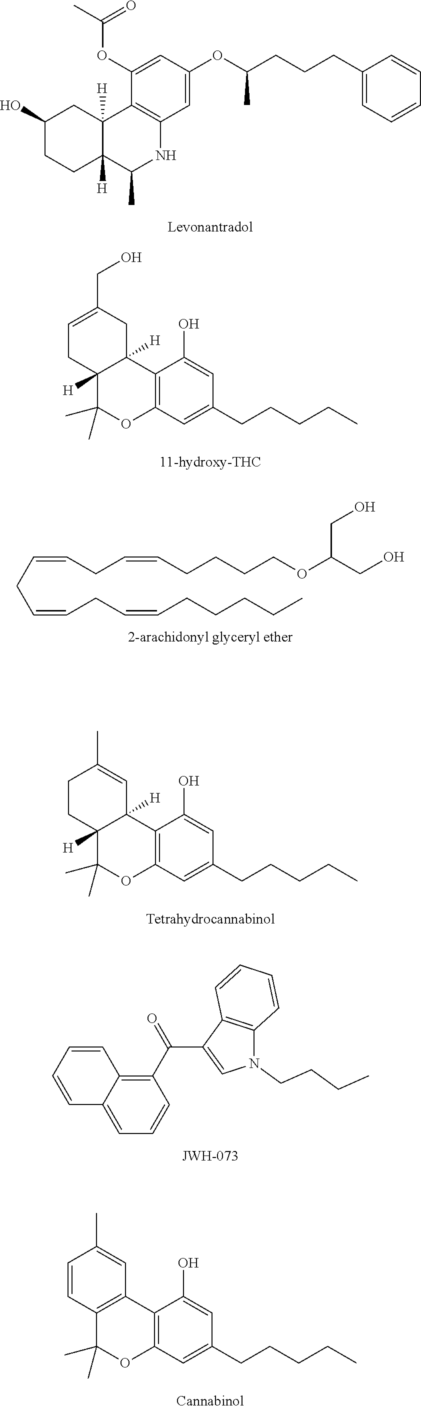

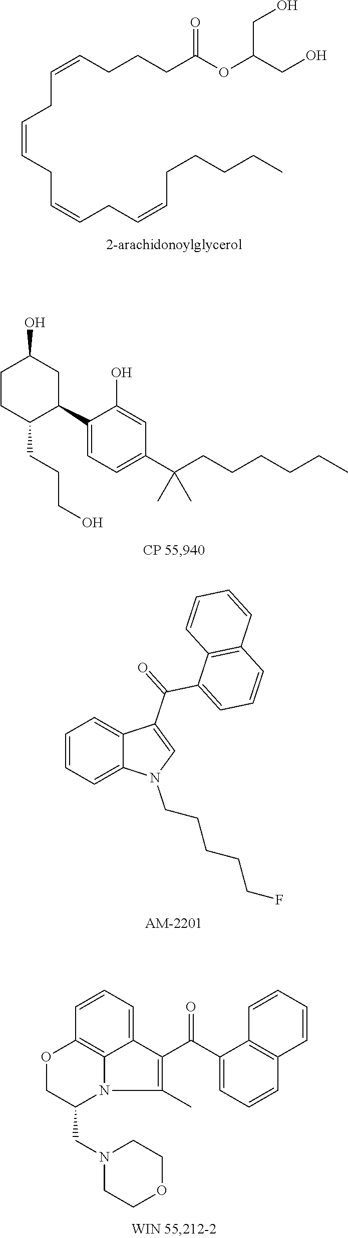

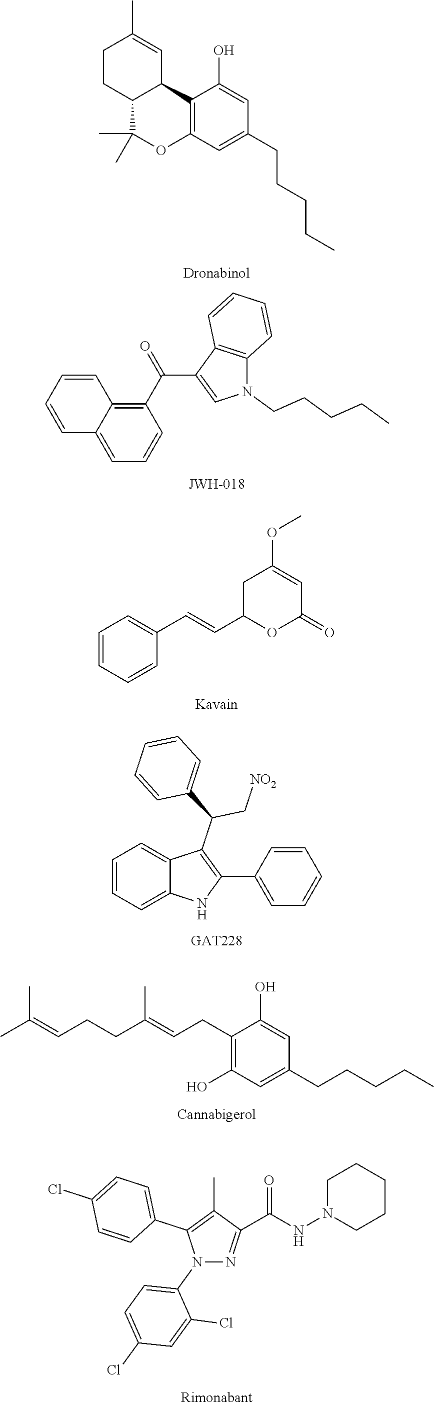

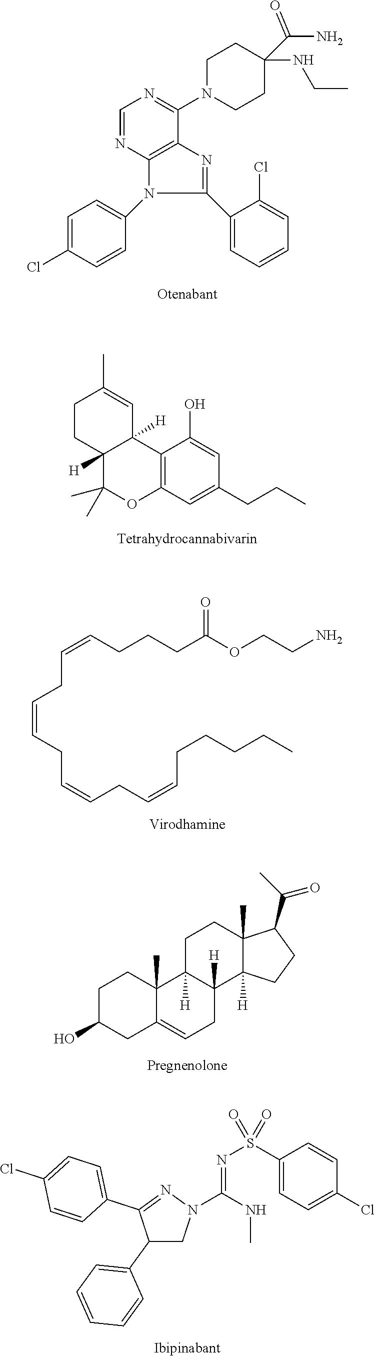

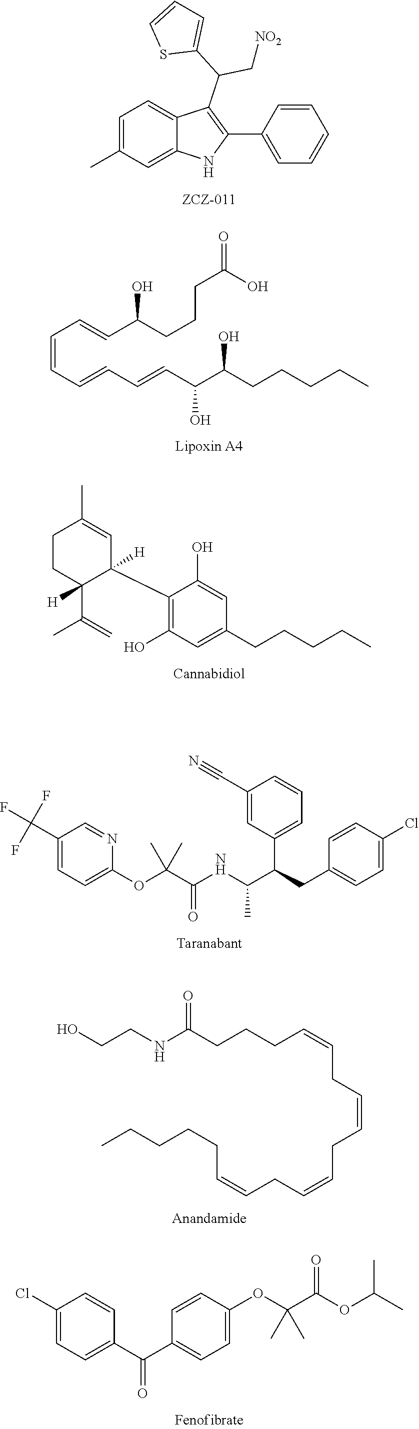 [SMILES: Cc1ccc2c(C(C[N+](=O)[O-])c3cccs3)c(-c3ccccc3)[nH]c2c1 ; CCCCC[C@H](O)[C@H](O)/C=C/C=C/C=C\C=C\[C@@H](O)CCCC(=O)O ; C=C(C)[C@@H]1CCC(C)=C[C@H]1c1c(O)cc(CCCCC)cc1O ; C[C@H](NC(=O)C(C)(C)Oc1ccc(C(F)(F)F)cn1)[C@H](Cc1ccc(Cl)cc1)c1cccc(C#N)c1 ; CCCCC/C=C\C/C=C\C/C=C\C/C=C\CCCC(=O)NCCO ; CC(C)OC(=O)C(C)(C)Oc1ccc(C(=O)c2ccc(Cl)cc2)cc1]

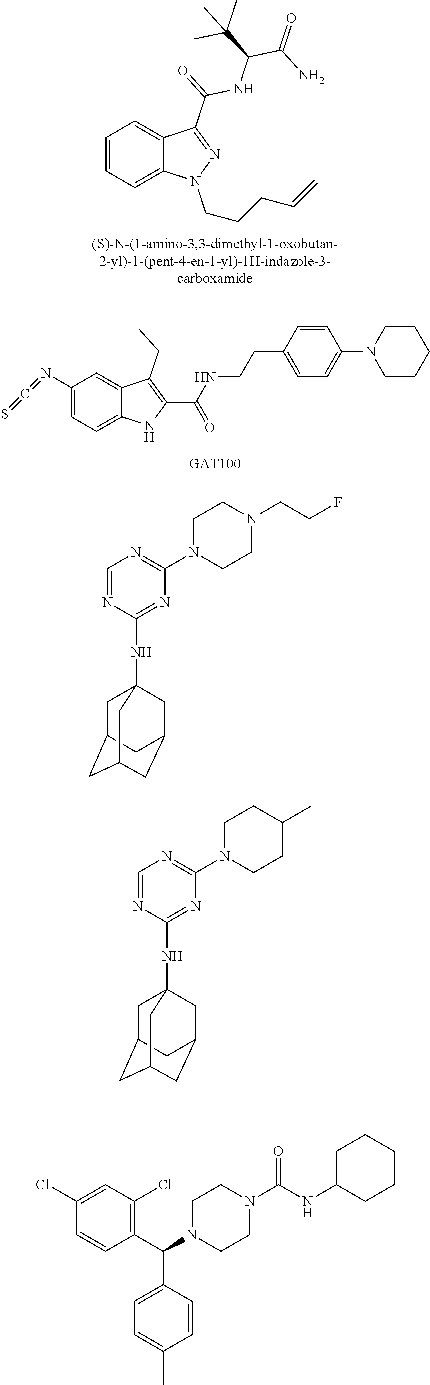 [SMILES: C=CCCCn1nc(C(=O)N[C@H](C(N)=O)C(C)(C)C)c2ccccc21 ; CCc1c(C(=O)NCCc2ccc(N3CCCCC3)cc2)[nH]c2ccc(N=C=S)cc12 ; FCCN1CCN(c2ncnc(NC34CC5CC(CC(C5)C3)C4)n2)CC1 ; CC1CCN(c2ncnc(NC34CC5CC(CC(C5)C3)C4)n2)CC1 ; Cc1ccc([C@@H](c2ccc(Cl)cc2Cl)N2CCN(C(=O)NC3CCCCC3)CC2)cc1]

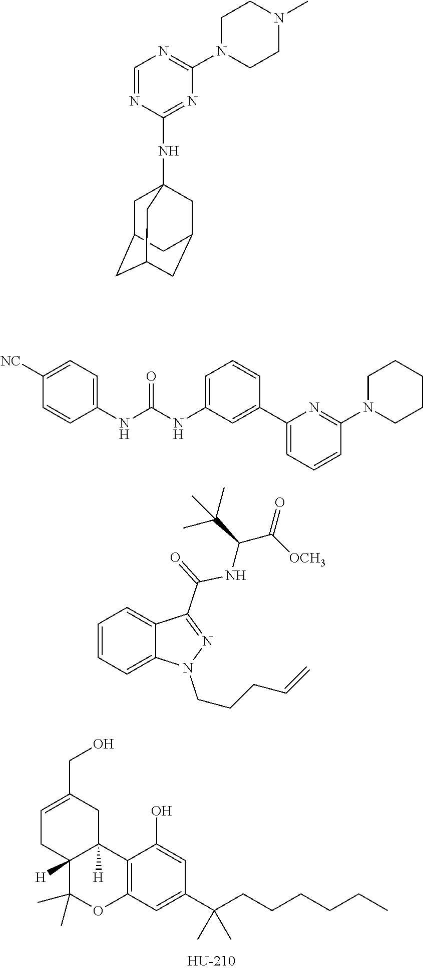

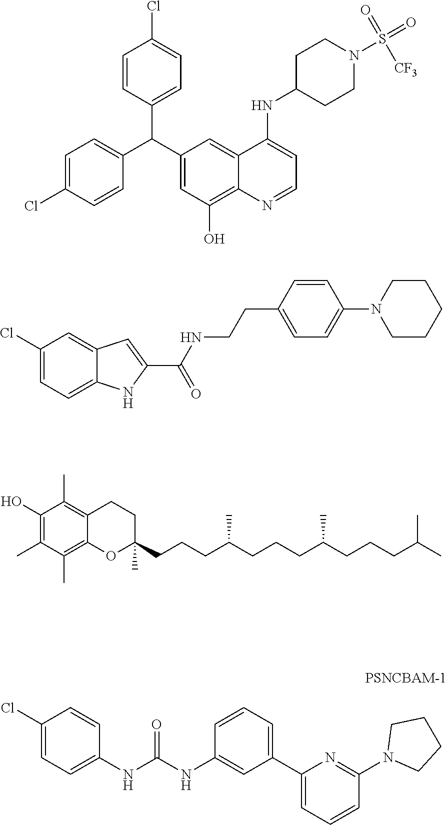 [SMILES: O=S(=O)(N1CCC(Nc2ccnc3c(O)cc(C(c4ccc(Cl)cc4)c4ccc(Cl)cc4)cc23)CC1)C(F)(F)F ; O=C(NCCc1ccc(N2CCCCC2)cc1)c1cc2cc(Cl)ccc2[nH]1 ; Cc1c(C)c2c(c(C)c1O)CC[C@@](C)(CCC[C@H](C)CCC[C@H](C)CCCC(C)C)O2 ; O=C(Nc1ccc(Cl)cc1)Nc1cccc(-c2cccc(N3CCCC3)n2)c1]

In some embodiments, a CB₁ ligand is RVD-Hpa (a fragment of hemoglobin (pepcan-12), alpha 1).

In certain embodiments, a CB₁ ligand is (S)—N-(1-amino-3,3-dimethyl-1-oxobutan-2-yl)-1-(pent-4-en-1-yl)-1H-indazole-3-carboxamide. In certain embodiments, the CB₁ ligand is anandamide.

In some embodiments, a CB₁ ligand is an anti-CB₁ antibody. In certain embodiments, a CB₁ ligand is an anti-CB₁ antibody fragment, or an anti-CB₁ antibody variant. An “anti-CB₁ antibody” refers to an immune system protein that recognizes, binds to, or otherwise interacts with a CB₁ receptor.

In certain embodiments, a CB₁ ligand is conjugated (e.g., linked, connected, attached, associated with) to and one or more agent moieties. In certain embodiments, the agent moiety is a therapeutic, prophylactic, diagnostic, or imaging agent. In certain embodiments, the agent is a small molecule or oligomeric compound. In certain embodiments, the agent moiety is protein, peptide, antibody, oligonucleotide, small molecule, large molecule or combination thereof.

In some embodiments, more than one CB₁ ligand is conjugated to an agent moiety. In some embodiments, at least two CB₁ ligands (e.g., 2, 3, 4, 5, 6, 7, 8, 9, 10, or more CB₁ ligands) are conjugated to an agent moiety. In some embodiments, two CB₁ ligands are conjugated to an agent moiety. In some embodiments, three CB₁ ligands are conjugated to an agent moiety. In some embodiments, four CB₁ ligands are conjugated to an agent moiety. In some embodiments, five CB₁ ligands are conjugated to an agent moiety. In some embodiments, more than five CB₁ ligands are conjugated to an agent moiety. In some embodiments, at least one to about five CB₁ ligands are conjugated to an agent moiety. In some embodiments, at least one to about four CB₁ ligands are conjugated to an agent moiety. In some embodiments, at least one to about three CB₁ ligands are conjugated to an agent moiety. In some embodiments, at least one to about two CB₁ ligands are conjugated to an agent moiety.

When an agent moiety is conjugated to multiple CB₁ ligands, all of the CB₁ ligands may be conjugated at or near the same position on the agent moiety, or the CB₁ ligands may be conjugated to multiple different positions on the agent moiety.

In some embodiments, an oligonucleotide is conjugated (e.g., connected, attached, associated with) to a CB₁ ligand through either a 5′ end and/or a 3′ end of the oligonucleotide, or at an internal position in an oligonucleotide (i.e., at a nucleotide on the oligonucleotide other than the 5′ or 3′ nucleotide). In some embodiments, an oligonucleotide is conjugated to a CB₁ ligand through the 5′ end of the oligonucleotide. In some embodiments, an oligonucleotide is conjugated to a CB₁ ligand through the 3′ end of the oligonucleotide. In some embodiments, an oligonucleotide is conjugated to CB₁ ligands through both the 5′ end and the 3′ end of the oligonucleotide. In some embodiments, an oligonucleotide is conjugated to a CB₁ ligand at an internal position within the oligonucleotide (e.g., in an “internally-modified oligonucleotide”).

In some embodiments, an oligonucleotide is conjugated to more than one CB₁ ligand. In some embodiments, an oligonucleotide is conjugated to at least two CB₁ ligands (e.g., 2, 3, 4, 5, 6, 7, 8, 9, 10, or more CB₁ ligands). In some embodiments, an oligonucleotide is conjugated to two CB₁ ligands. In some embodiments, an oligonucleotide is conjugated to three CB₁ ligands. In some embodiments, an oligonucleotide is conjugated four CB₁ ligands. In some embodiments, an oligonucleotide is conjugated to five CB₁ ligands. In some embodiments, an oligonucleotide is conjugated to more than five CB₁ ligands. In some embodiments, an oligonucleotide is conjugated to at least one to about five CB₁ ligands. In some embodiments, an oligonucleotide is conjugated to at least one to about four CB₁ ligands. In some embodiments, an oligonucleotide is conjugated to at least one to about three CB₁ ligands. In some embodiments, an oligonucleotide is conjugated to at least one to about two CB₁ ligands.

When an oligonucleotide is conjugated to multiple CB₁ ligands, all of the CB₁ ligands may be conjugated at or near the same position on the oligonucleotide, or the CB₁ ligands may be conjugated to multiple different positions on the oligonucleotide. In some embodiments, multiple CB₁ ligands (i.e., two, three, four, five, or more CB₁ ligands) are conjugated at the 5′ end of the oligonucleotide. In some embodiments, multiple CB₁ ligands (i.e., two, three, four, five, or more CB₁ ligands) are conjugated at the 3′ end of the oligonucleotide. In some embodiments, multiple CB₁ ligands (i.e., two, three, four, five, or more CB₁ ligands) are conjugated at one or more internal positions of the oligonucleotide. In some embodiments, an oligonucleotide is conjugated to one or more CB₁ ligands at the 5′ end of the oligonucleotide and/or one or more CB₁ ligands at the 3′ end of the oligonucleotide and/or one or more CB₁ ligands at an internal position, or multiple internal positions, of the oligonucleotide.

### Linkers

In certain embodiments, conjugates of the compound formulae described herein are provided. In certain embodiments, the conjugates comprise a CB₁ ligand covalently coupled to an agent moiety. In certain embodiments, the conjugates provided herein comprise one or more linker moieties. In certain embodiments, the one or more linker moieties link a CB₁ ligand to an agent moiety. In certain embodiments, the agent moiety is a protein, peptide, antibody, nucleic acid, small molecule, large molecule, therapeutic, prophylactic, diagnostic, or imaging agent. In some embodiments, a compound is conjugated to an oligonucleotide. In certain embodiment a CB₁ ligand is conjugated to an oligonucleotide. In certain embodiments, a compound comprises one or more CB₁ ligands, one or more linker moieties and one or more agent moieties, wherein the CB₁ ligands are conjugated (e.g., linked, connected, attached, associated with) to the one of more agent moieties through one or more linker moieties.

Conjugates as disclosed herein can be manufactured using any available method. When associating compounds provided herein with agent moieties (e.g., a CB₁ ligand with an oligonucleotide), the moieties may be linked directly or indirectly (e.g., through a linker moiety; that is, the linker is covalently bonded to each of the oligonucleotide and the CB₁ ligand; in some formulae herein “-Lₙ-” wherein n is a number (e.g., L₁, L₂, L₃, L₄, L₅, L₆, and L₇)). For example, the oligonucleotide and CB₁ ligand may be directly associated with one another, e.g., by one or more covalent bonds, or may be associated by means of one or more linkers. A “linker” refers to any chemical moiety (e.g., a combination of atoms having appropriate valency according to known chemistry principles) used to conjugate two components of the compounds provided herein (e.g., a CB₁ ligand and an oligonucleotide) to one another. Each of the two components may be connected to any portion of any of the linkers provided herein. In some embodiments, one component of the compounds provided herein (e.g., a CB₁ ligand or an oligonucleotide) is connected by a bond to one end of a linker, and the other component is connected by a bond to the other end of the linker. In some embodiments, one or both components of the compounds provided herein may be connected by a bond to an internal position within any of the linkers described herein. For example, in the context of an “alkyl linker,” a CB₁ ligand may be joined by a bond to a carbon at one end of the alkyl linker, and an oligonucleotide may be joined by a bond to a carbon at the other end of the alkyl linker. In some embodiments, a linker is a bond (including, e.g., phosphodiester and phosphorothioate bonds). In some embodiments, a linker is an optionally substituted alkyl linker (i.e., an alkyl chain is used to join two moieties, which may each be conjugated to opposite ends of the alkyl linker, or one or both moieties may be conjugated to an internal carbon on the alkyl linker). In some embodiments, a linker is an optionally substituted polyethylene glycol (PEG) linker (i.e., a PEG chain is used to join two moieties, which may each be conjugated to opposite ends of the PEG linker, or one or both moieties may be conjugated to an internal position on the PEG linker). In some embodiments, a linker is an optionally substituted heteroalkyl linker (i.e., a heteroalkyl chain is used to join two moieties, which may each be conjugated to opposite ends of the heteroalkyl linker, or one or both moieties may be conjugated to an internal position on the heteroalkyl linker). In some embodiments, a linker is an optionally substituted heteroaryl linker (i.e., a heteroaryl group is used to join two moieties, which may each be conjugated to any position on the heteroaryl group).

In certain embodiments, the compounds provided herein comprise one or more linking groups. In certain embodiments, each of L₁, L₂, L₃, and L₄ comprises a linking group. In certain embodiments, each of L₁, L₂, L₃, L₄, and L₅ comprises a linking group. In certain embodiments, each of L₁, L₂, L₃, L₄, L₅, L₆, and L₇ comprises a linking group. In certain embodiments, a linking group is covalently bound to a CB₁ ligand. In certain embodiments, a linking group is covalently bound to an oligonucleotide. In certain embodiments, a linking group is covalently bound to a cleavable moiety. In certain embodiments, a linking group comprises a cleavable bond. In certain embodiments, a linking group does not comprise a cleavable moiety. In certain embodiments, a linking group comprises a covalent attachment to a solid support. In certain embodiments, a linking group includes multiple positions for attachment of CB₁ ligands.

In certain embodiments, a linking group comprises a chain structure, such as a hydrocarbyl chain, or an oligomer of repeating units or combination of such repeating units. In certain embodiments, a linking group comprises 1 to 50 repeating units, 1 to 40 repeating units, 1 to 25 repeating units, 1 to 20 repeating units, 1 to 15 repeating units, 1 to 10 repeating units, or 1 to 5 repeating units. In certain embodiments, a linking group is 1 to 50 atoms long, 1 to 40 atoms long, 1 to 25 atoms long, 1 to 20 atoms long, 1 to 15 atoms long, 1 to 10 atoms long, or 1 to 5 atoms long.

In certain embodiments, a linking group contains carbon atoms. In certain embodiments, a linking group contains heteroatoms (e.g., nitrogen, oxygen, sulfur, etc.). In certain embodiments, a linking group forms amide linkages, ester linkages, or disulfide linkages. In certain embodiments, a linking group forms hydrazone linkages, oxime linkages, imine linkages, guanidine linkages, urea linkages, carbamate linkages, unsaturated alkyl linkages, sulfonamide linkages or 4-8 membered hetero cyclic linkages. In certain embodiments, a linking group comprises one or more groups selected from alkyl, amino, oxo, amide, disulfide, polyethylene glycol, ether, thioether, and hydroxylamino. In certain embodiments, a linking group comprises at least one phosphorus group. In certain embodiments, a linking group comprises at least one phosphate group. In certain embodiments, a linking group includes at least one neutral linking group. In certain embodiments, a linking group is substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted.

In certain embodiments, a linking group includes, but is not limited to, substituted or unsubstituted C1-C10 alkyl, substituted or unsubstituted C2-C10 alkenyl, or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl. In certain embodiments, a linking group is an aliphatic or heteroaliphatic. For example, the linking group can a polyalkyl linking group. The linking group can be a polyether linking group. The linking group can be a polyethylene linking group, such as PEG.

In certain embodiments, the linking group is a short peptide chain. In certain embodiments, a linking group comprises 1 to 40 amino acids, 1 to 25 amino acids, 1 to 20 amino acids, 1 to 15 amino acids, 1 to 10 amino acids, or 1 to 5 amino acids.

In certain embodiments, a linking group comprises linker-nucleosides. In certain embodiments, a linking group comprises 1 to 40 linker-nucleosides, 1 to 25 linker-nucleosides, 1 to 20 linker-nucleosides, 1 to 15 linker-nucleosides, 1 to 10 linker-nucleosides, or 1 to 5 linker-nucleosides. In certain embodiments, such linker-nucleosides may be modified or unmodified nucleosides. It is typically desirable for linker-nucleosides to be cleaved from the compound after it reaches a target tissue. Accordingly, linker-nucleosides herein can be linked to one another and to the remainder of the compound through cleavable bonds. Herein, linker-nucleosides are not considered to be part of an oligonucleotide payload. Accordingly, in embodiments in which a compound comprises an oligonucleotide consisting of a specified number or range of linked nucleosides and/or a specified percent complementarity to a reference nucleic acid and the compound also comprises a CB₁ ligand comprising a linking group comprising linker-nucleosides, those linker-nucleosides are not counted toward the length of the oligonucleotide and are not used in determining the percent complementarity of the oligonucleotide for the reference nucleic acid.

In certain embodiments, the linking group includes a protein binding group. In certain embodiments, the protein binding group is a lipid such as for example including but not limited to cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, friedelin, epifriedelanol derivatized lithocholic acid), or a cationic lipid. In certain embodiments, the protein binding group is a C₁₆ to C₂₂ long chain saturated or unsaturated fatty acid, cholesterol, cholic acid, vitamin E, adamantane or 1-pentafluoropropyl.

In certain embodiments, a linking group includes, but is not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA).

In certain embodiments, a linking group includes, without limitation, those linking groups described in the following references: U.S. Pat. Nos. 5,994,517; 6,300,319; 6,660,720; 6,906,182; 7,262,177; 7,491,805; 8,106,022; 7,723,509; 9,127,276; US 2006/0148740; US 2011/0123520; WO2013/033230; WO2012/037254, Biessen et al., J. Med. Chem. 1995, 38, 1846-1852; Lee et al., Bioorganic & Medicinal Chemistry 2011, 19, 2494-2500; Rensen et al., J. Biol. Chem. 2001, 276, 37577-37584; Rensen et al., J. Med. Chem. 2004, 47, 5798-5808; Sliedregt et al., J. Med. Chem. 1999, 42, 609-618; Valentijn et al., Tetrahedron, 1997, 53, 759-770; Lee, Carhohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycohiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromh Vase Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 7,582,744; 8,552,163; 8,137,695; 6,908,903; 6,383,812; 7,262,177; 6,525,031; 5,994,517; 6,660,720; 6,300,319; 7,723,509; 8,106,022; 7,491,805; 7,491,805; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2003/0119724; US2011/0207799; US2012/0035115; US2012/0230938; US2005/0164235; US2006/0183886; US2012/0136042; US2012/0095075; US2013/0109817; US2006/0148740; US2008/0206869; US2012/0165393; US2012/0101148; US2013/0121954; US2011/0123520; US2003/0077829; US2008/0108801; and US2009/0203132; each of which is incorporated herein by reference in its entirety.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise a structure selected from among:

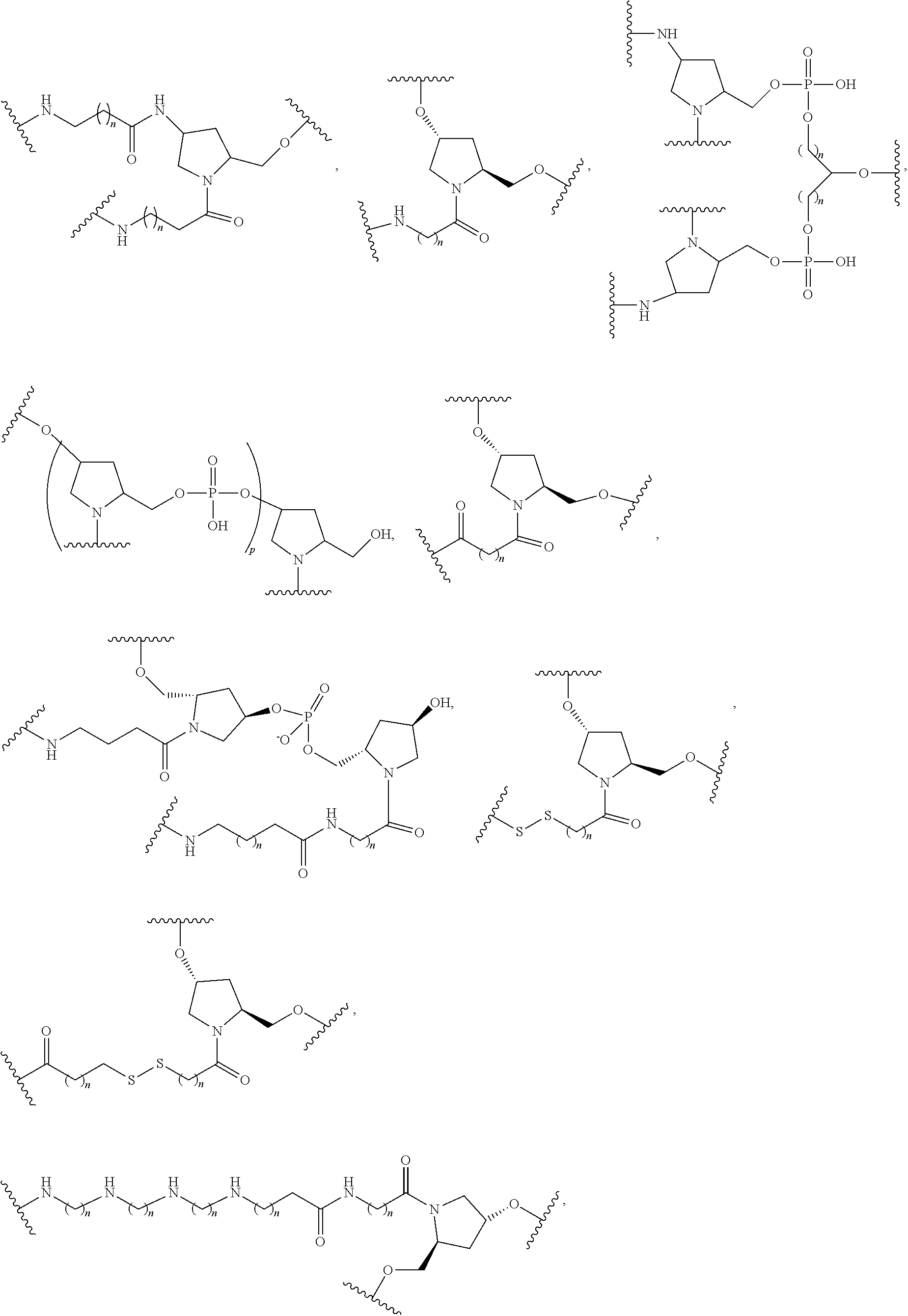

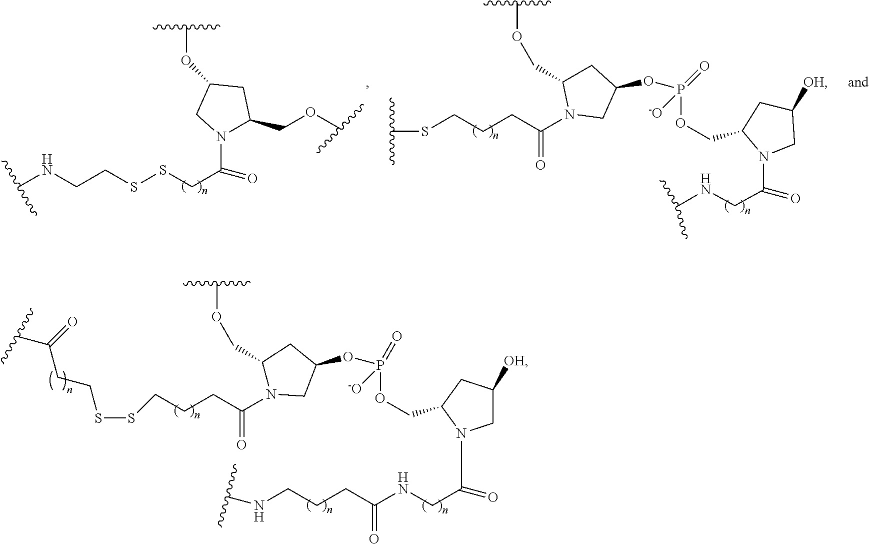

wherein each n is, independently, from 1 to 20; and p is from 1 to 6.

In certain embodiments, L₁, L₂, L₃, and L₄ (or L₁, L₂, L₃, L₄, and L₅, or L₁, L₂, L₃, L₄, L₅, L₆, and L) independently comprise or together comprise the structure selected from among:

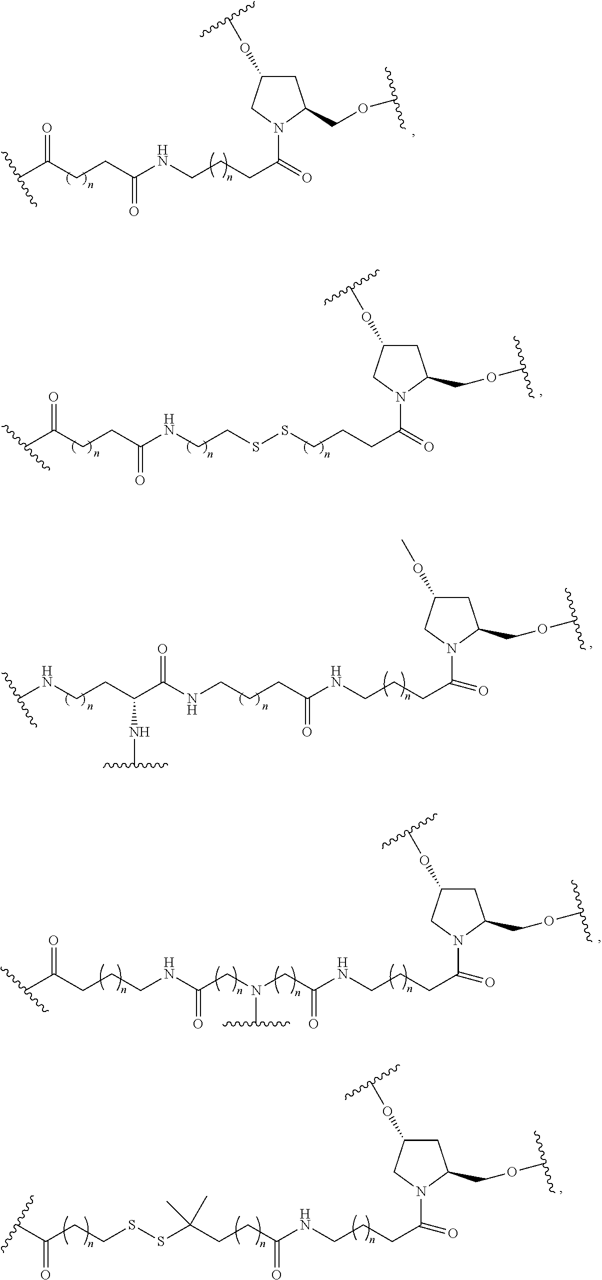

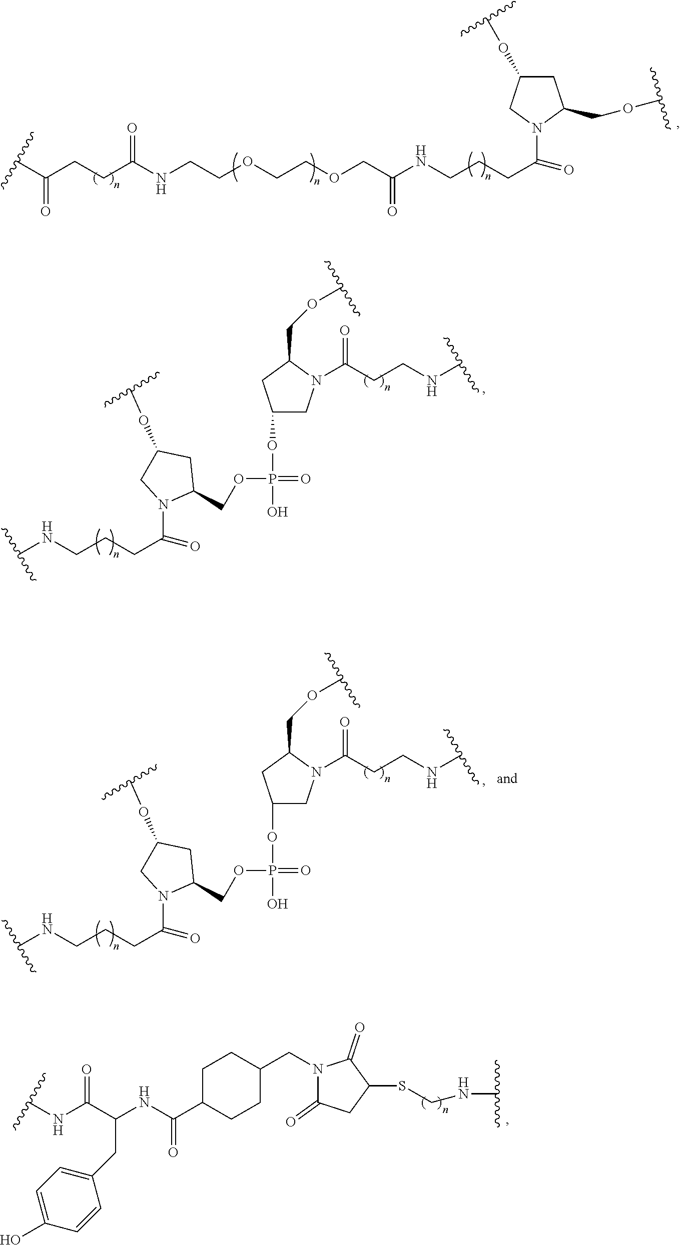

wherein each n is, independently, from 1 to 20.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

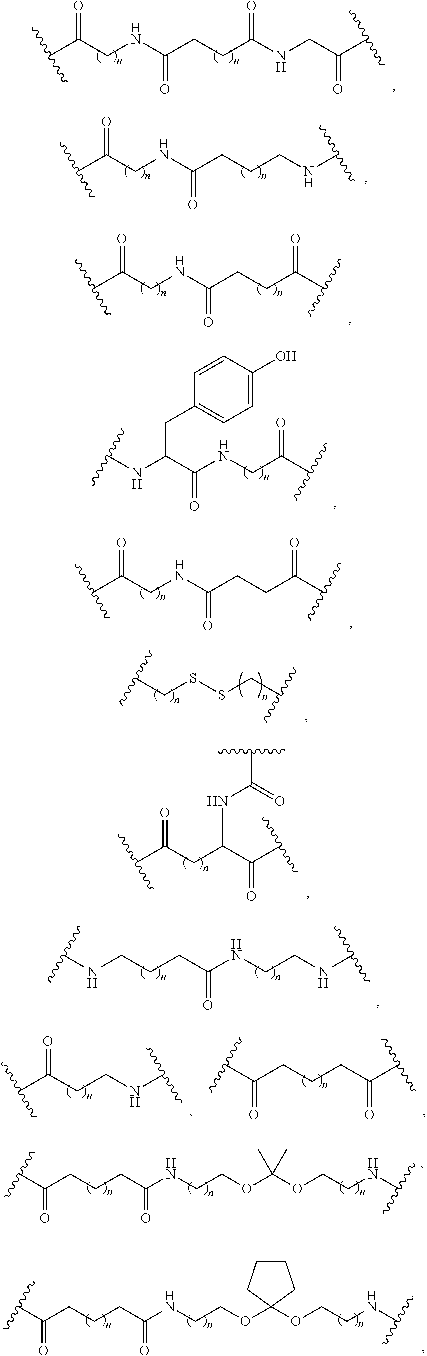

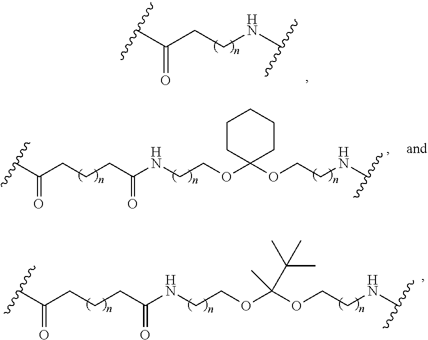

wherein each n is, independently, from 1 to 20.

In certain embodiments, L₁, L₂. L₃, and L₄ (or L₁, L₂, L₃, L₄, and L₅, or L₁, L₂, L₃, L₄, L₅, L₆, and L₇) independently comprise or together comprise the structure selected from among:

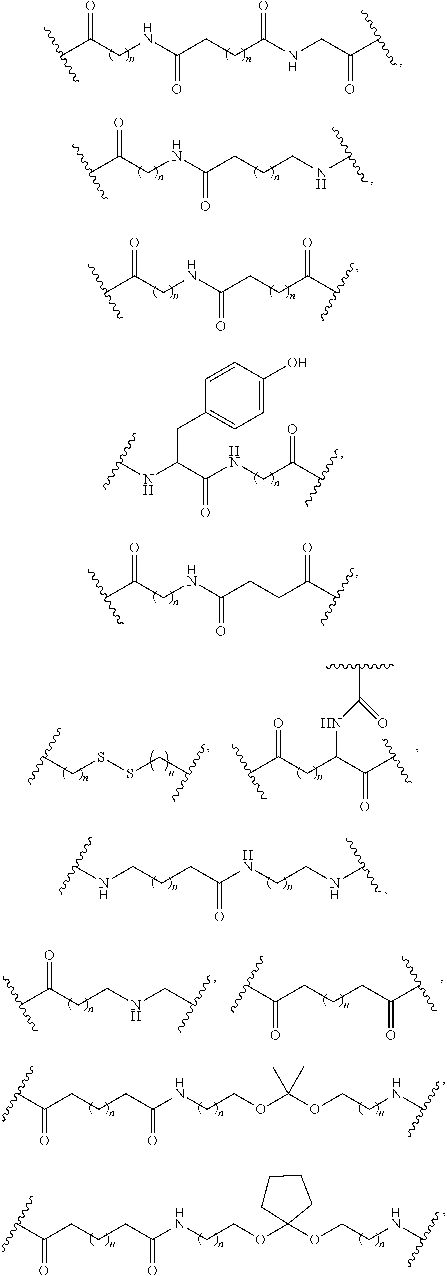

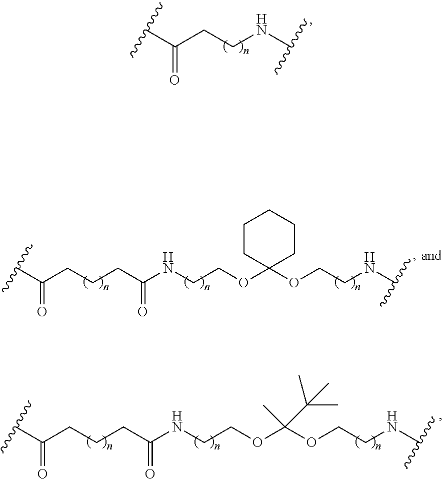

wherein each n is, independently, from 1 to 20.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

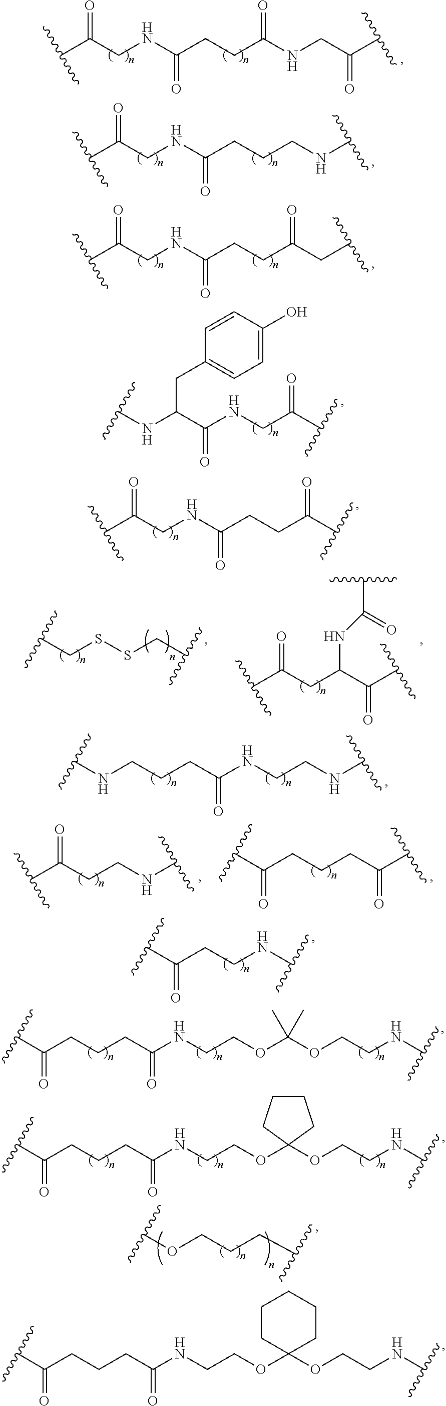

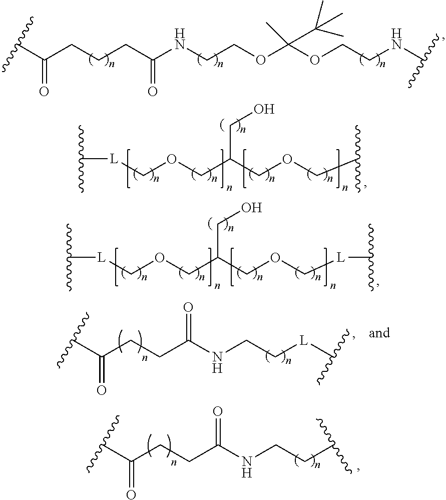

wherein each L is, independently, a phosphorous linking group; and each n is, independently, from 1 to 20.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

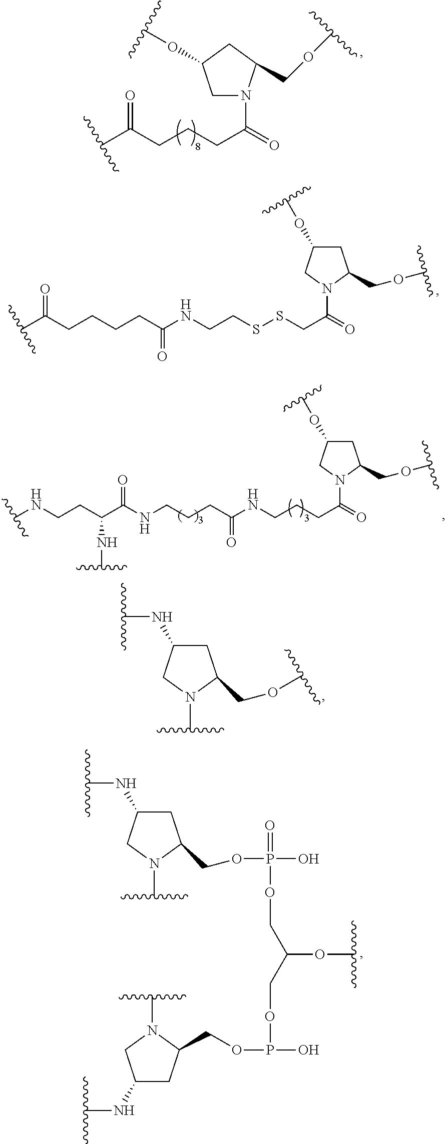

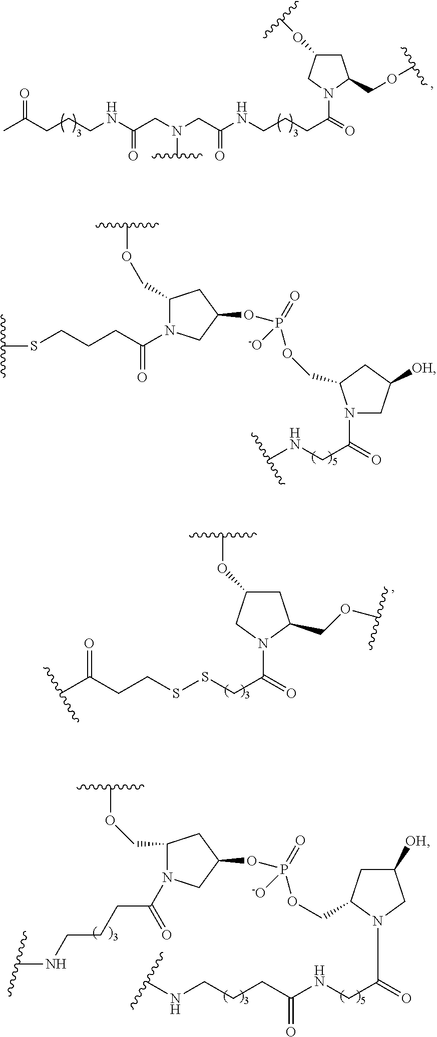

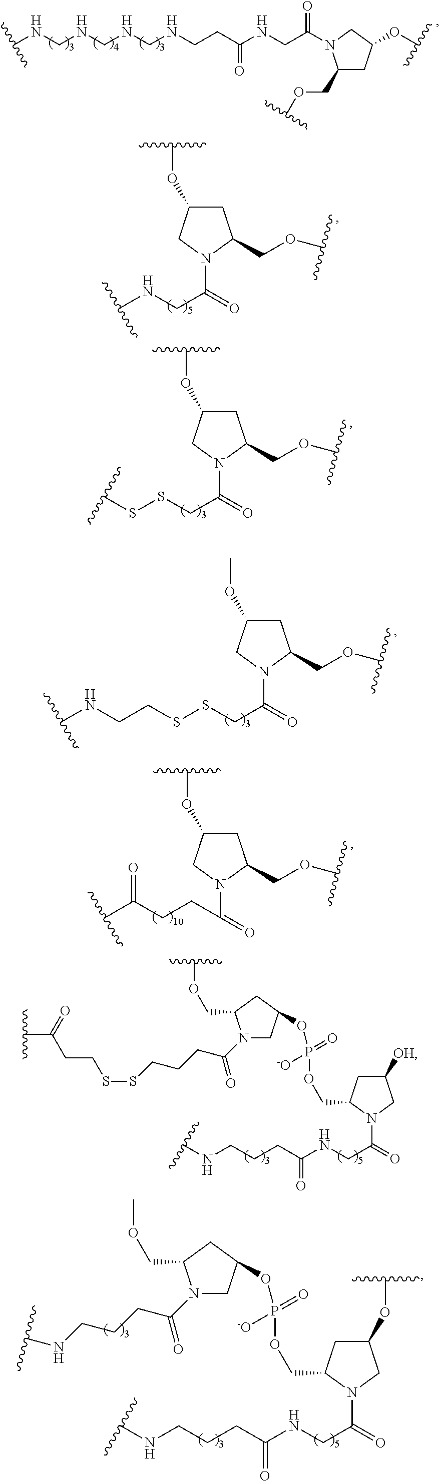

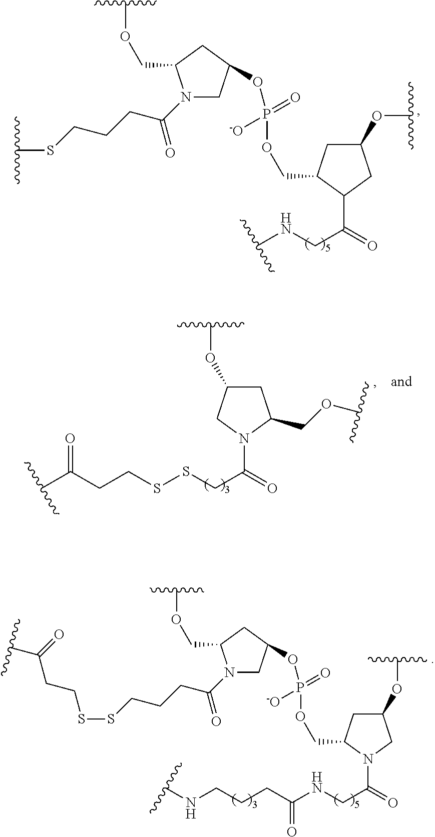

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

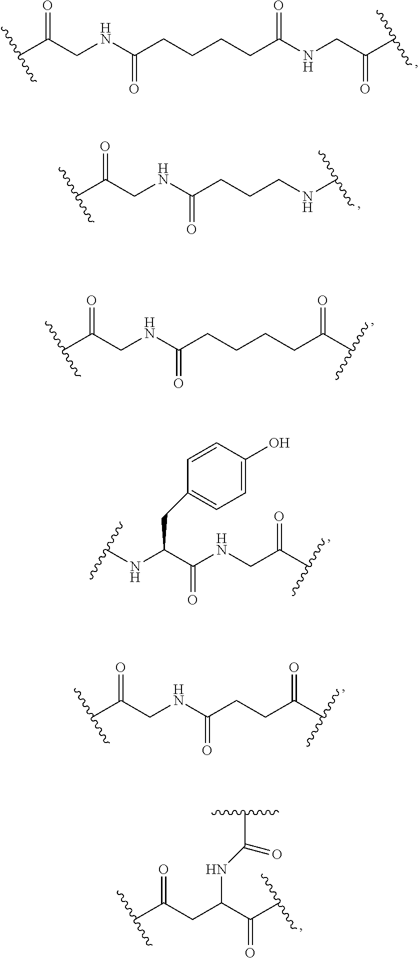

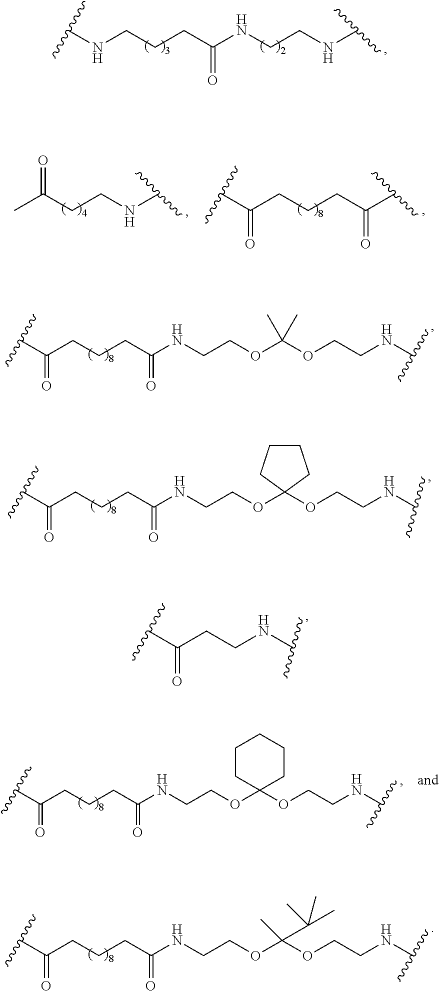

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

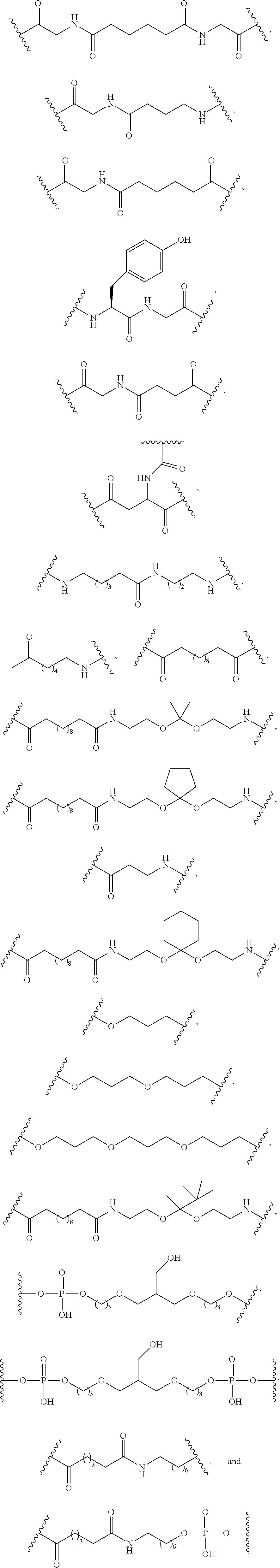

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

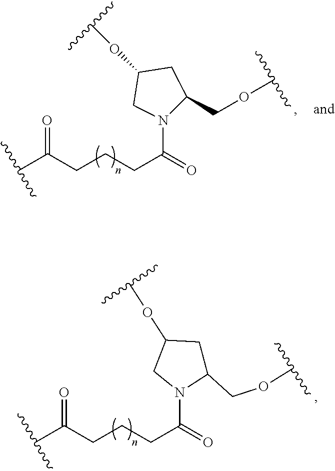

wherein n is from 1 to 20.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

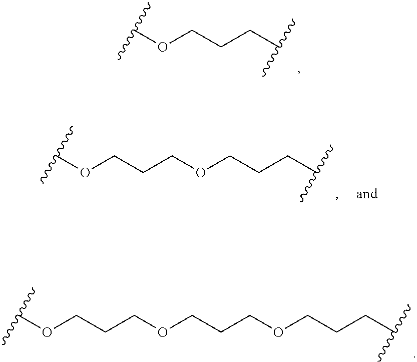 [SMILES: *CCCO* ; *CCCOCCCO* ; *CCCOCCCOCCCO*]

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

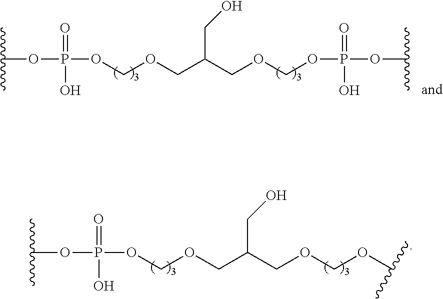

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

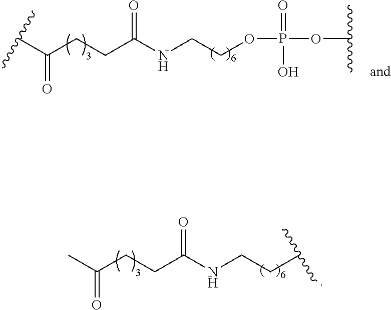

In certain embodiments, L₁, L₂, L₃, and L₄ (or L₁, L₂, L₃, L₄, and L₅, or L₁, L₂, L₃, L₄, L₅, L₆, and L) independently comprise or together have the structure:

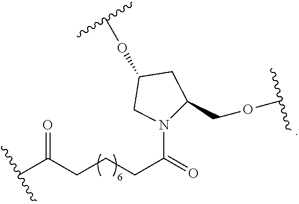

In certain embodiments, L₁, L₂, L₃, and L₄ (or L₁, L₂, L₃, L₄, and L₅, or L₁, L₂, L₃, L₄, L₅, L₆, and L₇) independently comprise or together have the structure:

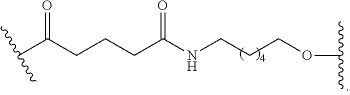

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

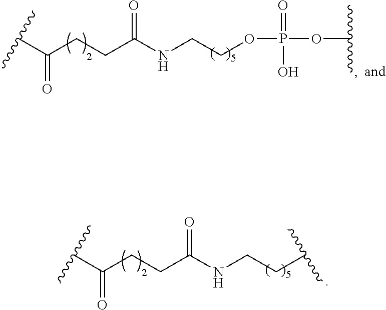

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

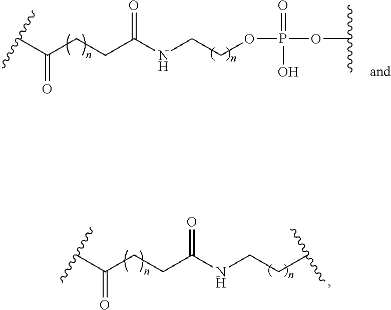

wherein each n is independently 0, 1, 2, 3, 4, 5, 6, or 7.

In some embodiments, any of L₁, L₂, L₃, and L₄ (or L₁, L₂, L₃, L₄, and L₅, or L₁, L₂, L₃, L₄, L₅, L₆, and L₇) may independently be a linker (e.g., an optionally substituted alkyl linker, an optionally substituted polyethylene glycol (PEG) linker, an optionally substituted heteroalkyl linker, or an optionally substituted heteroaryl linker). In some embodiments, any of L₁, L₂, L₃, and L₄ (or L₁, L₂, L₃, L₄, and L₅, or L₁, L₂, L₃, L₄, L₅, L₆, and L₇) may independently be a bond (e.g., a carbon-carbon bond, a phosphodiester bond, or a phosphorothioate bond). In some embodiments, any of L₁, L₂, L₃, and L₄ (or L₁, L₂, L₃, L₄, and L₅, or L₁, L₂, L₃, L₄, L₅, L₆, and L₇) may independently be absent.

In some embodiments, L₁ is a bond.

In some embodiments, L₂ is an optionally substituted PEG linker. In some embodiments, the PEG linker is three or four PEG units in length. In certain embodiments, L₂ comprises the structure

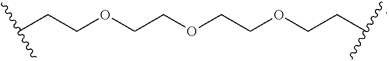 [SMILES: *CCOCCOCCOCC*]

In some embodiments, the PEG linker is two or three PEG units in length.

In some embodiments, L₃ is an optionally substituted heteroaryl linker. In some embodiments, L₃ is an optionally substituted partially unsaturated heteroaryl linker. In certain embodiments, L₃ comprises the structure

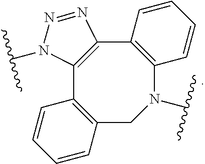 [SMILES: *N1Cc2ccccc2-c2c(nnn2*)-c2ccccc21]

In some embodiments, L₄ is an optionally substituted heteroalkyl linker. In some embodiments, the heteroalkyl linker is substituted with one or more ═O substituents. In certain embodiments, L₄ comprises the structure

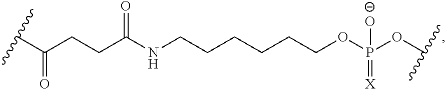

wherein X is O or S.

In some embodiments, L₁, L₂, L₃, and L₄ together comprise the structure

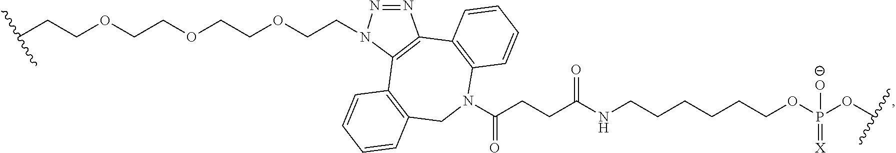

wherein X is O or S.

In some embodiments, one of L₃ and L₄ is a phosphodiester bond or a phosphorothioate bond, and the other of L₃ and L₄ is a bond. In certain embodiments, L₁, L₂, L₃, and L₄ together comprise the structure

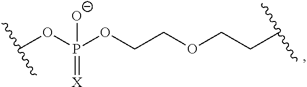

wherein X is O or S.

In certain embodiments, L₁, L₂, L₃, and L₄ together comprise the structure

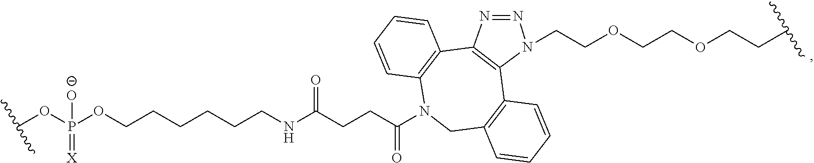

wherein X is O or S.

In some embodiments, any of L₁, L₂, L₃, L₄, and L₅ may independently be a linker (e.g., an optionally substituted alkyl linker, an optionally substituted polyethylene glycol (PEG) linker, an optionally substituted heteroalkyl linker, or an optionally substituted heteroaryl linker). In some embodiments, any of L₁, L₂, L₃, L₄, and L₅ may independently be a bond (e.g., a carbon-carbon bond, a phosphodiester bond, or a phosphorothioate bond). In some embodiments, any of L₁, L₂, L₃, L₄, and L₅ may independently be absent.

In some embodiments, L₁ and L₅ are each an optionally substituted PEG linker. In some embodiments, L₁ and L₅ are each an optionally substituted PEG linker three PEG units in length.

In some embodiments, L₂ is an optionally substituted heteroalkyl linker. In some embodiments, the heteroalkyl linker is substituted with one or more ═O substituents. In certain embodiments, L₂ comprises the structure

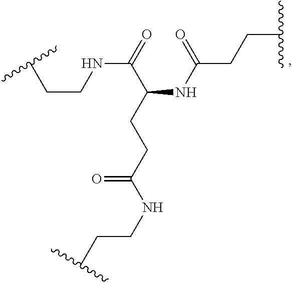 [SMILES: *CCNC(=O)CC[C@H](NC(=O)CC*)C(=O)NCC*]

In some embodiments, L₃ is an optionally substituted heteroaryl linker. In some embodiments, L₃ is an optionally substituted partially unsaturated heteroaryl linker. In certain embodiments, L₃ comprises the structure

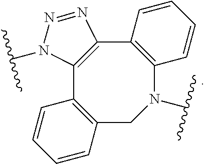 [SMILES: *N1Cc2ccccc2-c2c(nnn2*)-c2ccccc21]

In some embodiments, L₄ is an optionally substituted heteroalkyl linker. In some embodiments, the heteroalkyl linker is substituted with one or more ═O substituents. In certain embodiments, L₄ comprises the structure

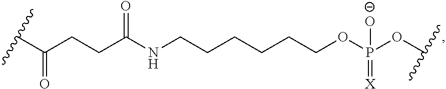

wherein X is O or S.

In some embodiments, L₁, L₂, L₃, L₄, and L₅ together comprise the structure

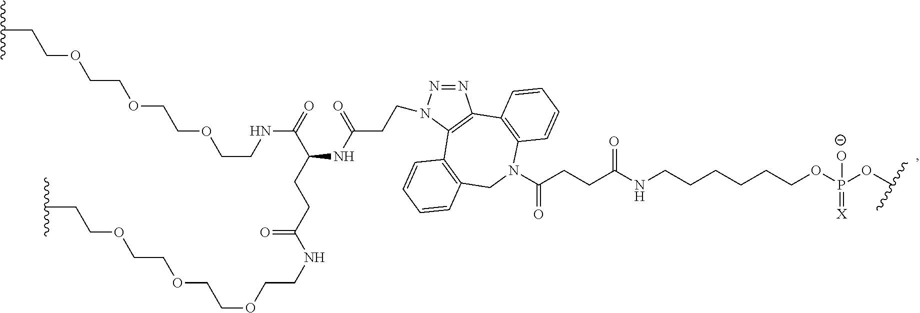

wherein X is O or S.

In some embodiments, any of L₁, L₂, L₃, L₄, L₅, L₆, and L₇ may independently be a linker (e.g., an optionally substituted alkyl linker, an optionally substituted polyethylene glycol (PEG) linker, an optionally substituted heteroalkyl linker, or an optionally substituted heteroaryl linker). In some embodiments, any of L₁, L₂, L₃, L₄, L₅, L₆, and L₇ may independently be a bond (e.g., a carbon-carbon bond, a phosphodiester bond, or a phosphorothioate bond). In some embodiments, any of L₁, L₂, L₃, L₄, L₅, L₆, and L₇ may independently be absent.

In some embodiments, L₁ is an optionally substituted PEG linker. In certain embodiments, L₁ is an optionally substituted PEG linker two or three PEG units in length.

In some embodiments, L₂ and L₅ are each independently an optionally substituted PEG linker. In some embodiments, L₂ and L₅ are each independently an optionally substituted PEG linker three or four PEG units in length. In certain embodiments, L₁, L₂, and L₅ together comprise the structure

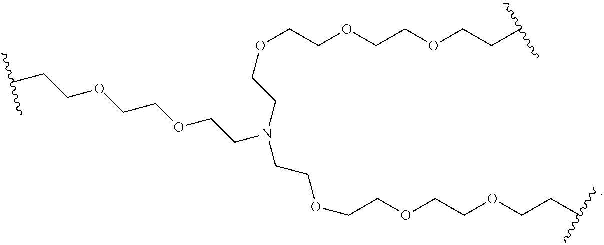 [SMILES: *CCOCCOCCOCCN(CCOCCOCC*)CCOCCOCCOCC*]

In some embodiments, L₃ and L₆ are each independently an optionally substituted heteroaryl linker. In some embodiments, L₃ and L₆ are each independently an optionally substituted partially unsaturated heteroaryl linker. In certain embodiments, L₃ and L₆ each comprise the structure

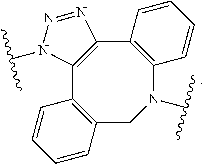 [SMILES: *N1Cc2ccccc2-c2c(nnn2*)-c2ccccc21]

In some embodiments, L₄ and L₇ are each independently an optionally substituted heteroalkyl linker. In some embodiments, the heteroalkyl linker is substituted with one or more ═O substituents. In certain embodiments, L₄ and L₇ each comprise the structure

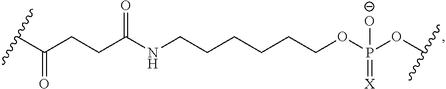

wherein X is O or S.

In some embodiments, L₁, L₂, L₃, L₄, L₅, L₆, and L₇ together comprise the structure

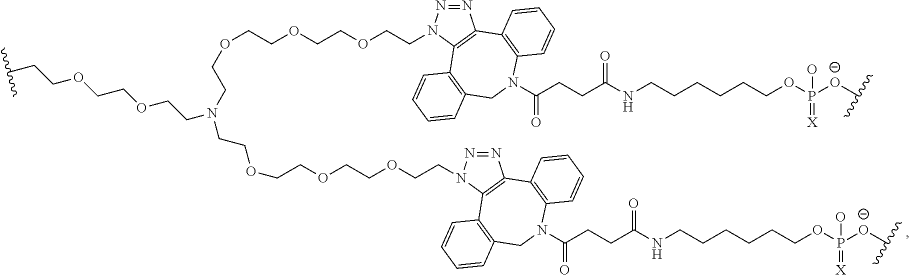

wherein X is O or S.

### Methods of Making Compounds

In some aspects, the disclosure relates to methods of making the compounds and compositions comprising CB₁ ligands as disclosed herein.

Compounds of the present disclosure can be made by means known in the art of organic synthesis. Methods for optimizing reaction conditions, and minimizing competing by-products, if necessary, are known in the art. Reaction optimization and scale-up may advantageously utilize high-speed parallel synthesis equipment and computer-controlled microreactors (e.g., Design and Optimization in Organic Synthesis, 2^{nd} Edition, Carlson R, Ed, 2005; Elsevier Science Ltd.; Jahnisch, K et al., Angew. Chem. Int. Ed. Engl. 2004 43: 406; and references therein). Additional reaction schemes and protocols may be determined by the skilled artisan by use of commercially available structure-searchable database software, for instance, SciFinder® (CAS division of the American Chemical Society) and Reaxys® (Elsevier), or by appropriate keyword searching using an internet search engine such as Google® or keyword databases such as the U.S. Patent and Trademark Office text database.

As can be appreciated by the skilled artisan, methods of synthesizing the compounds of the formulae herein will be evident to those of ordinary skill in the art, including in the schemes and examples herein. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present disclosure.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds described herein, even though only a single tautomeric form may be represented. All such isomeric forms of such compounds herein are expressly included in the present disclosure. All crystal forms and polymorphs of the compounds described herein are expressly included in the present disclosure. Also embodied are extracts and fractions comprising compounds of the present disclosure. The term “isomers” is intended to include diastereoisomers, enantiomers, regioisomers, structural isomers, rotational isomers, tautomers, and the like. For compounds which contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

Preferred enantiomerically enriched compounds have an enantiomeric excess of 50% or more. More preferably, the compound has an enantiomeric excess of 60%, 70%, 80%, 90%, 95%, 98%, 99%, or more. In preferred embodiments, only one enantiomer or diastereomer of a chiral compound of the present disclosure is administered to cells or a subject.

### Methods of Treatment

In one aspect, provided are methods of treating a subject suffering from or susceptible to a disorder or disease, comprising administering to the subject an effective amount of a compound or pharmaceutical composition described herein.

In other aspects, provided are methods of treating a subject suffering from or susceptible to a disorder or disease, wherein the subject has been identified as in need of modulation of the function of a protein, comprising administering to said subject in need thereof, an effective amount of a compound or pharmaceutical composition described herein, such that said subject is treated for said disorder.

In one aspect, provided are methods of delivering a therapeutic oligonucleotide to the brain of a subject, comprising contacting the subject with a compound or pharmaceutical composition described herein, in an amount and under conditions sufficient to target the brain. In some embodiments, the therapeutic oligonucleotide is delivered to one or more brain regions selected from the group consisting of the striatum, the cerebellum, the brain stem, the hippocampus, the frontal cortex, and the spinal cord.

In certain embodiments, provided are methods of treating a disease, disorder or symptom thereof, wherein the disease is a central nervous system (CNS) disease, disorder, or symptom thereof. In some embodiments, the disease is a neurodegenerative disease, disorder, or symptom thereof. In some embodiments, the disease is Alzheimer's disease, or a symptom thereof.

Exemplary CNS disorders include, but are not limited to, neurotoxicity and/or neurotrauma, stroke, multiple sclerosis, spinal cord injury, epilepsy, a mental disorder, a sleep condition, a movement disorder, nausea and/or emesis, amyotrophic lateral sclerosis, Alzheimer's disease, and drug addiction.

In certain embodiments, the CNS disorder is neurotoxicity and/or neurotrauma, e.g., for example, as a result of acute neuronal injury (e.g., traumatic brain injury (TBI), stroke, epilepsy) or a chronic neurodegenerative disorder (e.g., multiple sclerosis, Parkinson's disease, Huntington's disease, amyotrophic lateral sclerosis, Alzheimer's disease). In certain embodiments, the compounds of the present disclosure provide a neuroprotective effect, e.g., against an acute neuronal injury or a chronic neurodegenerative disorder.

In certain embodiments, the CNS disorder is stroke (e.g., ischemic stroke).

In certain embodiments, the CNS disorder is multiple sclerosis.

In certain embodiments, the CNS disorder is spinal cord injury.

In certain embodiments, the CNS disorder is epilepsy.

In certain embodiments, the CNS disorder is a mental disorder, e.g., for example, depression, anxiety or anxiety-related conditions, a learning disability, somatic symptom disorder, schizophrenia, or schizoaffective disorder.

In certain embodiments, the CNS disorder is depression. “Depression” includes, but is not limited to, depressive disorders or conditions, such as, for example, major depressive disorders (e.g., unipolar depression), treatment-resistant depression, dysthymic disorders (e.g., chronic, mild depression), bipolar disorders (e.g., manic-depression), seasonal affective disorder, and/or depression associated with substance abuse or substance abuse disorder (e.g., withdrawal). The depression can be clinical or subclinical depression. The depression can be associated with or premenstrual syndrome and/or premenstrual dysphoric disorder.

In certain embodiments, the CNS disorder is anxiety. “Anxiety” includes, but is not limited to, anxiety and anxiety-related conditions, such as, for example, clinical anxiety, panic disorder, agoraphobia, generalized anxiety disorder (GAD), specific phobia, social phobia, obsessive-compulsive disorder, acute stress disorder, post-traumatic stress disorder, adjustment disorders with anxious features, anxiety disorder associated with depression, anxiety disorder due to general medical conditions, and substance-induced anxiety disorders, anxiety associated with substance abuse or substance use disorder (e.g., withdrawal, dependence, reinstatement) and anxiety associated with nausea and/or emesis. This treatment may also be to induce or promote sleep in a subject (e.g., for example, a subject with anxiety).

In certain embodiments, the CNS disorder is a learning disorder (e.g., attention deficit disorder (ADD)).

In certain embodiments, the CNS disorder is schizophrenia or schizoaffective disorder.

In certain embodiments, the CNS disorder is a sleep condition. “Sleep conditions” include, but are not limited to, insomnia, narcolepsy, sleep apnea, restless legs syndrome (RLS), delayed sleep phase syndrome (DSPS), periodic limb movement disorder (PLMD), hypopnea syndrome, rapid eye movement behavior disorder (RBD), shift work sleep condition (SWSD), and sleep problems (e.g., parasomnias) such as nightmares, night terrors, sleep talking, head banging, snoring, and clenched jaw and/or grinding of teeth (bruxism).

In certain embodiments, the CNS disorder is a movement disorder, e.g., basal ganglia disorders, such as, for example, Parkinson's disease, levodopa-induced dyskinesia, Huntington's disease, Gilles de Ta Tourette's syndrome, tardive dyskinesia, and dystonia.

In certain embodiments, the CNS disorder is Alzheimer's disease.

In certain embodiments, the CNS disorder is amyotrophic lateral sclerosis (ALS).

In certain embodiments, the CNS disorder is nausea and/or emesis.

In certain embodiments, the CNS disorder is drug addiction (e.g., for instance, addiction to opiates, nicotine, cocaine, psychostimulants, or alcohol).

The term “neurological disease” (including, e.g., “neurodegenerative diseases”) refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle, and neuromuscular junctions. Substance abuse or substance use disorder (SUD) and mental illness, including, but not limited to, bipolar disorder, schizophrenia, and schizoaffective disorder are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Amold-Chiari malformation; arteriovenous malformation; Asperger syndrome; ataxia telangiectasia; attention deficit hyperactivity disorder; autism; autonomic dysfunction; back pain; Batten disease; Behcet's disease: Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (also known as motor neuron disease or amyotrophic lateral sclerosis); lumbar disc disease; lyme disease-neurological sequelae; Machado-Joseph disease; macrencephaly; megalencephaly; Melkersson-Rosenthal syndrome; Meniere's disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy: progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

In certain embodiments, the subject is a mammal, preferably a primate or a human.

In another embodiment, provided are methods as described above, wherein the effective amount of the compounds provided herein is as described above.

In another embodiment, provided are methods as described above, wherein the compounds provided herein is administered intrathecally, intravenously, intramuscularly, subcutaneously, intracerebroventricularly, orally, or topically. In certain embodiments, the compound is administered intrathecally.

In other embodiments, provided are methods as described above, wherein the compound of any of the formulae provided herein is administered alone or in combination with one or more other therapeutics. In a further embodiment, the additional therapeutic agent is a central nervous system (CNS) disease agent.

Another object of the present disclosure is the use of a compound as described herein in the manufacture of a medicament for use in the treatment of a disorder or disease. Another object of the present disclosure is the use of a compound as described herein for use in the treatment of a disorder or disease.

### Pharmaceutical Compositions

In one aspect, provided are pharmaceutical compositions comprising any of the compounds described herein and a pharmaceutically acceptable carrier or pharmaceutically acceptable excipient.

A compound or composition, as described herein, can be administered in combination with one or more additional therapeutic agents (e.g., therapeutically and/or prophylactically active agents). The compounds or compositions can be administered in combination with additional therapeutic agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional therapeutic agent exhibits a synergistic effect that is absent in a pharmaceutical composition including one of the compounds described herein or the additional therapeutic agent, but not both.

The compound or composition can be administered concurrently with, prior to, or subsequent to one or more additional therapeutic agents, which may be useful as, e.g., combination therapies. Therapeutic agents include therapeutically active agents. Therapeutic agents also include prophylactically active agents. Therapeutic agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional therapeutic agent is a therapeutic agent useful for treating and/or preventing a disease (e.g., CNS disorder). Each additional therapeutic agent may be administered at a dose and/or on a time schedule determined for that therapeutic agent. The additional therapeutic agents may also be administered together with each other and/or with the compound or composition described herein in a single dose or administered separately in different doses. The particular combination to employ in a regimen will take into account compatibility of the compound described herein with the additional therapeutic agent(s) and/or the desired therapeutic and/or prophylactic effect to be achieved. In general, it is expected that the additional therapeutic agent(s) in combination be utilized at levels that do not exceed the levels at which they are utilized individually. In some embodiments, the levels utilized in combination will be lower than those utilized individually.

In one aspect, provided are kits comprising an effective amount of a compound provided herein, in unit dosage form, together with instructions for administering the compound to a subject suffering from or susceptible to a disease or disorder.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The present disclosure also provides a pharmaceutical composition, comprising an effective amount of a compound described herein and a pharmaceutically acceptable excipient. In an embodiment, a compound of any of the formulae provided herein is administered to a subject using a pharmaceutically acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of the disclosure may be varied so as to obtain an amount of the active ingredient which is effective to achieve the desired therapeutic response for a particular subject, composition, and mode of administration, while being acceptably tolerant to the subject.

In use, at least one compound according to the present disclosure is administered in a pharmaceutically effective amount to a subject in need thereof in a pharmaceutical carrier by intravenous, intrathecal, intramuscular, subcutaneous, or intracerebroventricular injection or by oral administration or topical application. In accordance with the present disclosure, a compound of the disclosure may be administered alone or in conjunction with a second, different therapeutic. By “in conjunction with” is meant together, substantially simultaneously, or sequentially. In one embodiment, a compound of the disclosure is administered acutely. The compound of the disclosure may therefore be administered for a short course of treatment, such as for about 1 day to about 1 week. In another embodiment, the compound of the disclosure may be administered over a longer period of time to ameliorate chronic disorders, such as, for example, for about one week to several months depending upon the condition to be treated.

By “pharmaceutically effective amount,” as used herein, is meant an amount of a compound of the disclosure, high enough to significantly positively modify the condition to be treated but low enough to avoid serious side effects (at a reasonable benefit/risk ratio), within the scope of sound medical judgment. A pharmaceutically effective amount of a compound of the disclosure will vary with the particular goal to be achieved, the age and physical condition of the patient being treated, the severity of the underlying disease, the duration of treatment, the nature of concurrent therapy and the specific compound employed. For example, a therapeutically effective amount of a compound of the disclosure administered to a child or a neonate will be reduced proportionately in accordance with sound medical judgment. The effective amount of a compound of the disclosure will thus be the minimum amount which will provide the desired effect.

A decided practical advantage of the present disclosure is that the compound may be administered in a convenient manner such as by intrathecal, intravenous, intramuscular, subcutaneous, oral, or intra-cerebroventricular injection routes or by topical application, such as in creams or gels. Depending on the route of administration, the active ingredients which comprise a compound of the disclosure may be required to be coated in a material to protect the compound from the action of enzymes, acids and other natural conditions which may inactivate the compound. In order to administer a compound of the disclosure by a mode other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

Some examples of substances which can serve as pharmaceutical excipients, or pharmaceutical carriers (which terms are used interchangeably herein), are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; tale; stearic acids; magnesium stearate; calcium sulfate; vegetable oils, such as peanut oils, cotton seed oil, sesame oil, olive oil, corn oil, and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, antioxidants, and preservatives, can also be present. Solubilizing agents, including for example, cremaphore, and beta-cyclodextrins, can also be used in the pharmaceutical compositions herein.

Pharmaceutical compositions comprising the active compounds of the present disclosure (or derivatives or prodrugs thereof) can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries, which facilitate processing of the active compounds into preparations that can be used pharmaceutically. The compositions herein can be made by combining (e.g., contacting, mixing, dissolving, granulating, dragee-making, levigating, emulsifying, encapsulating, entrapping, or lyophilizing) a compound delineated herein with one or more suitable carriers, diluents, excipients, or auxiliaries, including those described herein (e.g., for pharmaceutical, agricultural, or veterinary use).

Pharmaceutical compositions of the present disclosure can take a form suitable for virtually any mode of administration, including, for example, intrathecal, topical, ocular, oral, buccal, systemic, nasal, injection, transdermal, rectal, vaginal, and the like, or a form suitable for administration by inhalation or insufflation.

Systemic formulations include those designed for administration by injection, e.g., subcutaneous, intravenous, intramuscular, intrathecal, or intraperitoncal injection, as well as those designed for transdermal, transmucosal, oral, or pulmonary administration.

Useful injectable preparations include sterile suspensions, solutions, or emulsions of the active compound(s) in aqueous or oily vehicles. The compositions also can contain formulating agents, such as suspending, stabilizing and/or dispersing agent. The formulations for injection can be presented in unit dosage form (e.g., in ampules or in multidose containers) and can contain added preservatives.

Alternatively, the injectable formulation can be provided in powder form for reconstitution with a suitable vehicle, including but not limited to, sterile pyrogen free water, buffer, dextrose solution, and the like, before use. To this end, the active compound(s) can be dried by any art-known technique, such as lyophilization, and reconstituted prior to use.

For prolonged delivery, the active compound(s), or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt.

Alternatively, other pharmaceutical delivery systems can be employed. Liposomes and emulsions are well-known examples of delivery vehicles that can be used to deliver active compound(s), oligonucleotide(s), or prodrug(s). Certain organic solvents such as dimethylsulfoxide (DMSO) also can be employed.

The pharmaceutical compositions can, if desired, be presented in a pack or dispenser device that can contain one or more unit dosage forms containing the active compound(s). The pack can, for example, comprise metal or plastic foil, such as a blister pack. The pack or dispenser device can be accompanied by instructions for administration.

The active compound(s), or prodrug(s) of the present disclosure, or compositions thereof, will generally be used in an amount effective to achieve the intended result, for example in an amount effective to treat or prevent the particular disease being treated. The compound(s) and oligonucleotide(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. Therapeutic benefit also includes halting or slowing the progression of the disease, regardless of whether improvement is realized.

For prophylactic administration, the compound can be administered to a patient at risk of developing one of the previously described diseases. A patient at risk of developing a disease can be a patient having characteristics placing the patient in a designated group of at-risk patients, as defined by an appropriate medical professional or group. A patient at risk may also be a patient that is commonly or routinely in a setting where development of the underlying disease could occur. In other words, an at-risk patient is one who is commonly or routinely exposed to the disease or illness causing conditions or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, the mode of administration, whether the desired benefit is prophylactic or therapeutic, the severity of the indication being treated, the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC₅₀ of the particular compound as measured in an in vitro assay, such as an in vitro fungal MIC or MFC, and other in vitro assays. Calculating dosages to achieve such circulating blood or serum concentrations taking into account the bioavailability of the particular compound is well within the capabilities of skilled artisans. For guidance, see “General Principles,” In: Goodman and Gilman's The Pharmaceutical Basis of Therapeutics, Chapter 1, pp. 1-112, 13th ed., McGraw-Hill, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) that are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

Preferably, the compound(s) will provide therapeutic or prophylactic benefit and will have acceptable tolerability. Tolerability of the compound(s) and oligonucleotide(s) can be determined using standard pharmaceutical procedures. The dose ratio between non-tolerable and therapeutic (or prophylactic) effect is the therapeutic index. Compounds(s) that exhibit high therapeutic indices are preferred.

### ADDITIONAL EMBODIMENTS

Certain embodiments include embodiment P1 to embodiment P114 following.

Embodiment P1. A compound comprising the structure of Formula (I), or a salt thereof:

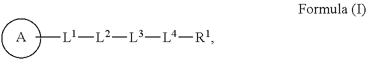

wherein

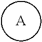

is a cannabinoid receptor type 1 (CB₁) ligand;
each of L₁, L₂, L₃, and L₄ is independently a linker, a bond, or absent; and
R¹ comprises one or more oligonucleotides, protecting groups, small molecules, proteins, antibodies, and/or peptides.

Embodiment P2. The compound, or a salt thereof, of embodiment P1, wherein the CB₁ ligand is a CB₁ agonist.

Embodiment P3. The compound, or a salt thereof, of embodiment P1, wherein the CB₁ ligand is a CB₁ antagonist.

Embodiment P4. The compound, or a salt thereof, of embodiment P1, wherein the CB₁ ligand is selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

Embodiment P5. The compound, or a salt thereof, of embodiment P1, wherein the CB₁ ligand comprises the structure

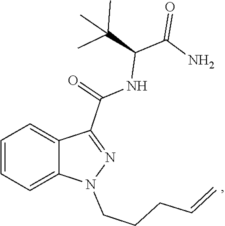 [SMILES: C=CCCCn1nc(C(=O)N[C@H](C(N)=O)C(C)(C)C)c2ccccc21]

or a derivative thereof.

Embodiment P6. The compound, or a salt thereof, of embodiment P1, wherein the compound comprises the structure of Formula (II):

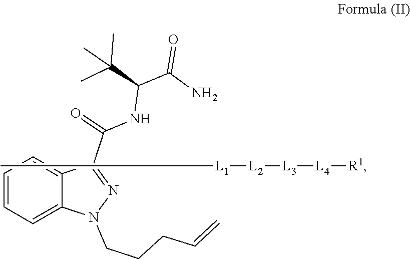

or a salt thereof.

Embodiment P7. The compound, or a salt thereof, of embodiment P6, wherein the compound comprises the structure of Formula (II-a):

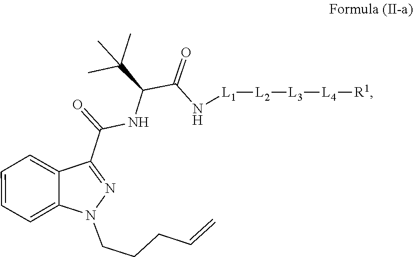

or a salt thereof

Embodiment P8. The compound, or a salt thereof, of any one of embodiments P1-P7, wherein each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, a phosphodiester bond, or a phosphorothioate bond.

Embodiment P9. The compound, or a salt thereof, of embodiment P8, wherein L₁ is a bond.

Embodiment P10. The compound, or a salt thereof, of embodiment P8 or P9, wherein L₂ is an optionally substituted PEG linker.

Embodiment P11. The compound, or a salt thereof, of embodiment P10, wherein the PEG linker is three or four PEG units in length.

Embodiment P12. The compound, or a salt thereof, of any one of embodiments P8-P11, wherein L₂ comprises the structure

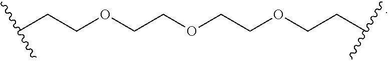 [SMILES: *CCOCCOCCOCC*]

Embodiment P13. The compound, or a salt thereof, of any one of embodiments P8-P12, wherein L₃ is an optionally substituted heteroaryl linker.

Embodiment P14. The compound, or a salt thereof, of embodiment P13, wherein L₃ is an optionally substituted partially unsaturated heteroaryl linker.

Embodiment P15. The compound, or a salt thereof, of embodiment P13 or P14, wherein L₃ comprises the structure

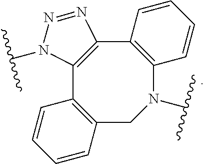 [SMILES: *N1Cc2ccccc2-c2c(nnn2*)-c2ccccc21]

Embodiment P16. The compound, or a salt thereof, of any one of embodiments P8-P15, wherein L₄ is an optionally substituted heteroalkyl linker.

Embodiment P17. The compound, or a salt thereof, of embodiment P16, wherein the heteroalkyl linker is substituted with one or more ═O substituents.

Embodiment P18. The compound, or a salt thereof, of embodiment P16 or P17, wherein L₄ comprises the structure

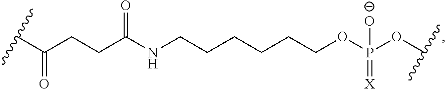

wherein X is O or S.

Embodiment P19. The compound, or a salt thereof, of any one of embodiments P8-P18, wherein L₁, L₂, L₃, and L₄ together comprise the structure

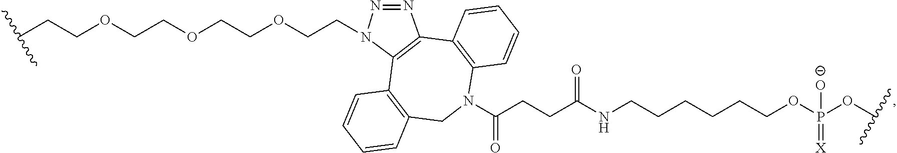

wherein X is O or S.

Embodiment P20. The compound, or a salt thereof, of any one of embodiments P1-P19, wherein the compound comprises the structure:

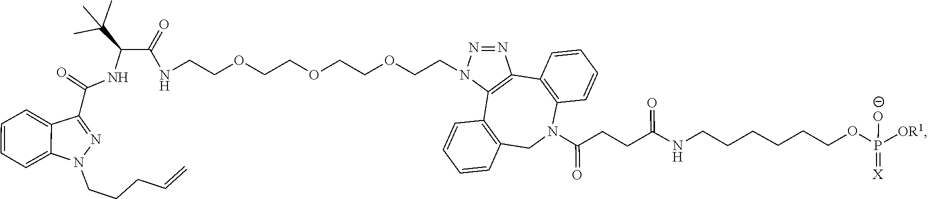

or a salt thereof, wherein X is O or S.

Embodiment P21. The compound, or a salt thereof, of any one of embodiments P18-P20, wherein X is O.

Embodiment P22. The compound, or a salt thereof, of any one of embodiments P18-P20, wherein X is S.

Embodiment P23. The compound, or a salt thereof, of embodiment P1, wherein the CB₁ ligand comprises the structure

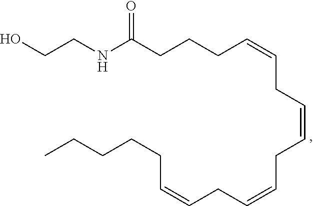 [SMILES: CCCCC/C=C\C/C=C\C/C=C\C/C=C\CCCC(=O)NCCO]

or a derivative thereof.

Embodiment P24. The compound, or a salt thereof, of embodiment P23, wherein the compound comprises the structure of Formula (III):

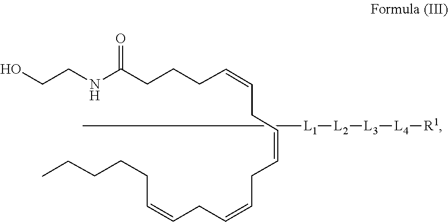

or a salt thereof.

Embodiment P25. The compound, or a salt thereof, of embodiment P24, wherein the compound comprises the structure of Formula (III-a):

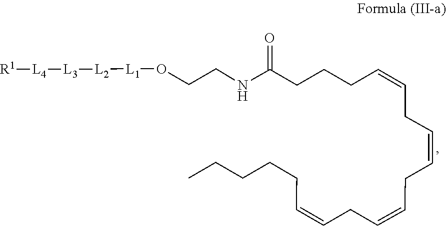

or a salt thereof.

Embodiment P26. The compound, or a salt thereof, of any one of embodiments P23-P25, wherein each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, a phosphodiester bond, or a phosphorothioate bond.

Embodiment P27. The compound, or a salt thereof, of embodiment P26, wherein L₁ is a bond.

Embodiment P28. The compound, or a salt thereof, of embodiment P26 or P27, wherein L₂ is an optionally substituted PEG linker.

Embodiment P29. The compound, or a salt thereof, of embodiment P28, wherein the PEG linker is two or three PEG units in length.

Embodiment P30. The compound, or a salt thereof, of any one of embodiments P26-P29, wherein one of L₃ and L₄ is a phosphodiester bond or a phosphorothioate bond, and the other of L₃ and L₄ is a bond.

Embodiment P31. The compound, or a salt thereof, of any one of embodiments P26-P30,

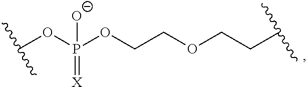

wherein L₁, L₂, L₃, and L₄ together comprise the structure wherein X is O or S.

Embodiment P32. The compound, or a salt thereof, of any one of embodiments P26-P29, wherein L₃ is an optionally substituted heteroaryl linker.

Embodiment P33. The compound, or a salt thereof, of embodiment P32, wherein L₃ is an optionally substituted partially unsaturated heteroaryl linker.

Embodiment P34. The compound, or a salt thereof, of embodiment P32 or P33, wherein L₃ comprises the structure

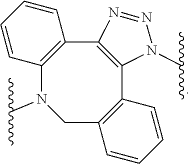 [SMILES: *N1Cc2ccccc2-c2c(nnn2*)-c2ccccc21]

Embodiment P35. The compound, or a salt thereof, of any one of embodiments P26-P29 or P32-P34, wherein L₄ is an optionally substituted heteroalkyl linker.

Embodiment P36. The compound, or a salt thereof, of embodiment P35, wherein the heteroalkyl linker is substituted with one or more ═O substituents.

Embodiment P37. The compound, or a salt thereof, of embodiment P35 or P36, wherein L₄ comprises the structure

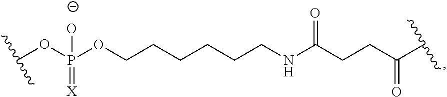

wherein X is O or S.

Embodiment P38. The compound, or a salt thereof, of any one of embodiments P26-P29 or P32-P37, wherein L₁, L₂, L₃, and L₄ together comprise the structure

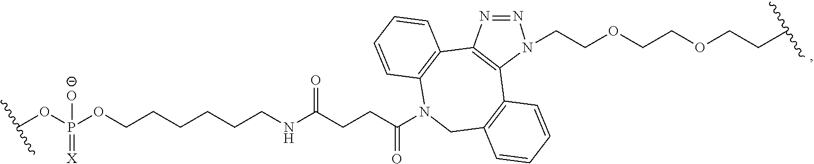

wherein X is O or S.

Embodiment P39. The compound, or a salt thereof, of any one of embodiments P23-P38, wherein the compound comprises the structure:

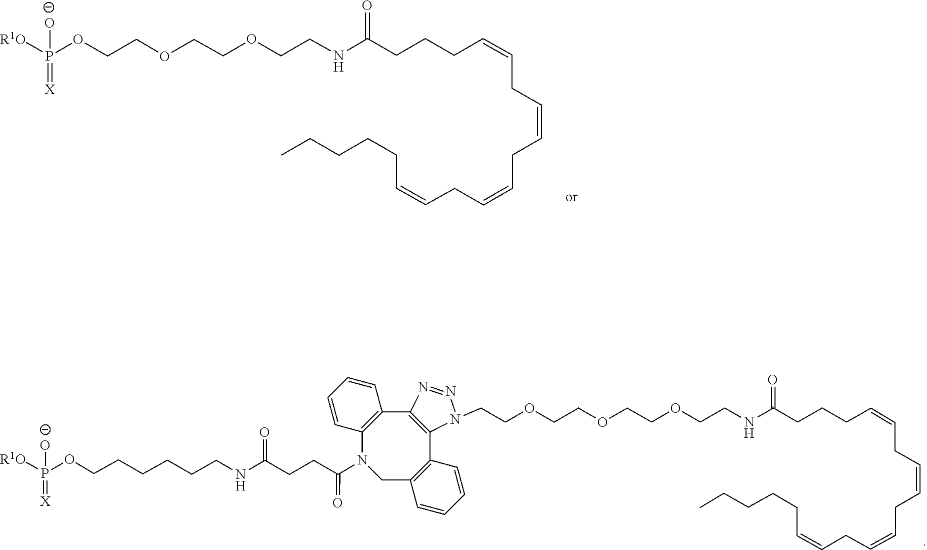

or a salt thereof, wherein X is O or S.

Embodiment P40. The compound, or a salt thereof, of any one of embodiments P23-P39, wherein X is O.

Embodiment P41. The compound, or a salt thereof, of any one of embodiments P23-P39, wherein X is S.

Embodiment P42. A compound comprising the structure of Formula (IV), or a salt thereof:

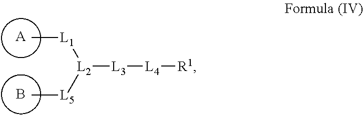

wherein

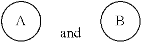

are each independently a cannabinoid receptor type 1 (CB₁) ligand, or one of

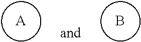

is a CB₁ ligand, and the other of

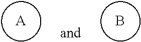

comprises a lipid or a ligand;
each of L₁, L₂, L₃, L₄, and L₅ is independently a linker, a bond, or absent; and
R¹ comprises one or more oligonucleotides, protecting groups, small molecules, proteins, antibodies, and/or peptides.

Embodiment P43. The compound, or a salt thereof, of embodiment P42, wherein

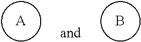

and are each independently a CB₁ ligand.

Embodiment P44. The compound, or a salt thereof, of embodiment P43, wherein one or both of the CB₁ ligands is a CB₁ agonist.

Embodiment P45. The compound, or a salt thereof, of embodiment P43, wherein one or both of the CB₁ ligands is a CB₁ antagonist.

Embodiment P46. The compound, or a salt thereof, of embodiment P43, wherein each of the CB₁ ligands is independently selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

Embodiment P47. The compound, or a salt thereof, of embodiment P43, wherein each of the CB₁ ligands comprises the structure

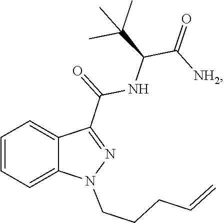 [SMILES: C=CCCCn1nc(C(=O)N[C@H](C(N)=O)C(C)(C)C)c2ccccc21]

or a derivative thereof.

Embodiment P48. The compound, or a salt thereof, of embodiment P43, wherein the compound comprises the structure of Formula (V):

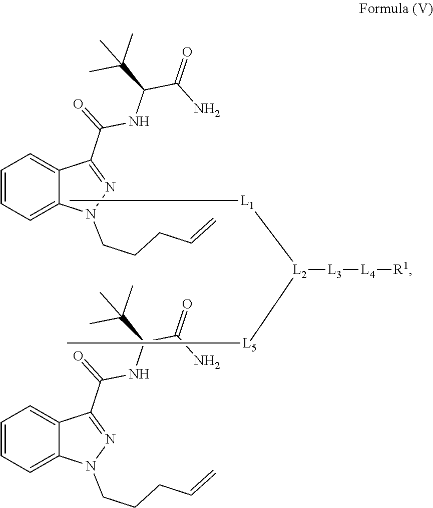

or a salt thereof.

Embodiment P49. The compound, or a salt thereof, of embodiment P48, wherein the compound comprises the structure of Formula (V-a):

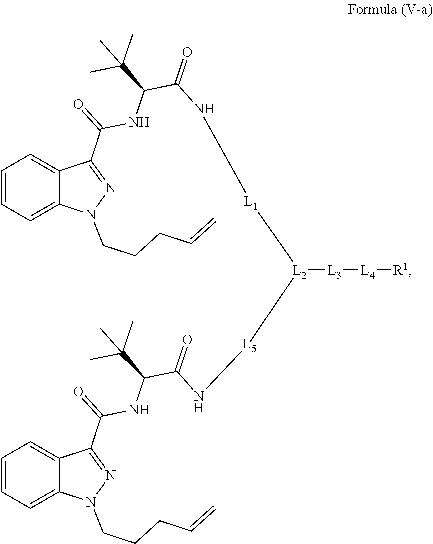

or a salt thereof.

Embodiment P50. The compound, or a salt thereof, of any one of embodiments P42-P49, wherein each of L₁, L₂, L₃, L₄, and L₅ is independently absent, a bond, an optionally substituted alkyl linker, an optionally substituted polyethylene glycol (PEG) linker, an optionally substituted heteroalkyl linker, an optionally substituted heteroaryl linker, a phosphodiester bond, or a phosphorothioate bond.

Embodiment P51. The compound, or a salt thereof, of embodiment P50, wherein L₁ and L₅ are each an optionally substituted PEG linker.

Embodiment P52. The compound, or a salt thereof, of embodiment P51, wherein L₁ and L₅ are each an optionally substituted PEG linker three PEG units in length.

Embodiment P53. The compound, or a salt thereof, of any one of embodiments P50-P52, wherein L₂ is an optionally substituted heteroalkyl linker.

Embodiment P54. The compound, or a salt thereof, of embodiment P53, wherein the heteroalkyl linker is substituted with one or more ═O substituents.

Embodiment P55. The compound, or a salt thereof, of any one of embodiments P50-P54, wherein L₂ comprises the structure

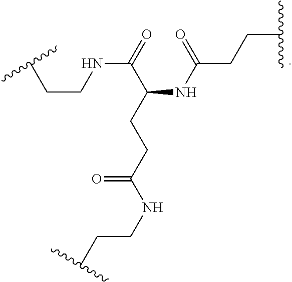 [SMILES: *CCNC(=O)CC[C@H](NC(=O)CC*)C(=O)NCC*]

Embodiment P56. The compound, or a salt thereof, of any one of embodiments P50-P55, wherein L₃ is an optionally substituted heteroaryl linker.

Embodiment P57. The compound, or a salt thereof, of embodiment P56, wherein L₃ is an optionally substituted partially unsaturated heteroaryl linker.

Embodiment P58. The compound, or a salt thereof, of embodiment P56 or P57, wherein L₃ comprises the structure

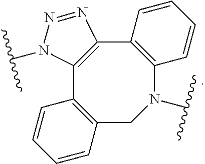 [SMILES: *N1Cc2ccccc2-c2c(nnn2*)-c2ccccc21]

Embodiment P59. The compound, or a salt thereof, of any one of embodiments P50-P58, wherein L₄ is an optionally substituted heteroalkyl linker.

Embodiment P60. The compound, or a salt thereof, of embodiment P59, wherein the heteroalkyl linker is substituted with one or more ═O substituents.

Embodiment P61. The compound, or a salt thereof, of embodiment P59 or P60, wherein L₄ comprises the structure

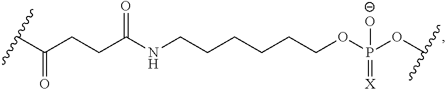

wherein X is O or S.

Embodiment P62. The compound, or a salt thereof, of any one of embodiments P50-P61, wherein L₁, L₂, L₃, L₄, and L₅ together comprise the structure

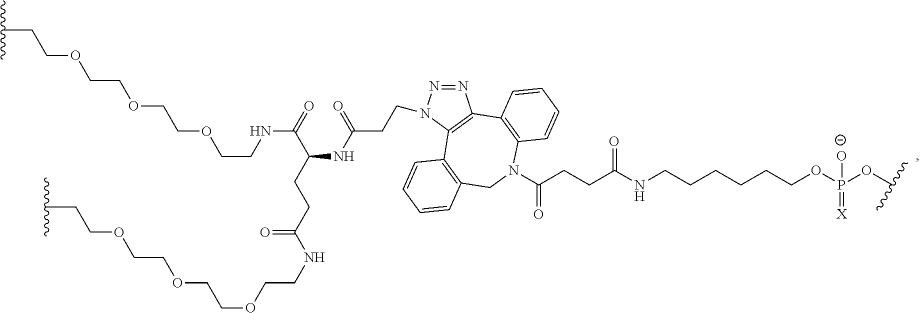

wherein X is O or S.

Embodiment P63. The compound, or a salt thereof, of any one of embodiments P42-P62, wherein the compound comprises the structure:

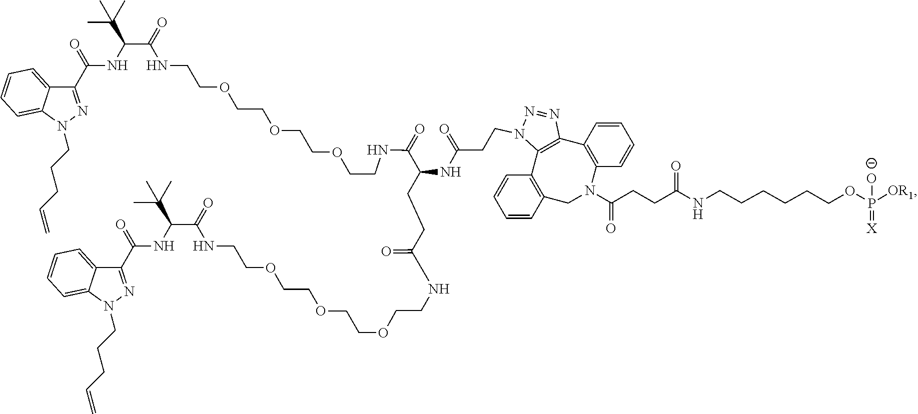

or a salt thereof, wherein X is O or S.

Embodiment P64. The compound, or a salt thereof, of any one of embodiments P42-P63, wherein X is O.

Embodiment P65. The compound, or a salt thereof, of any one of embodiments P42-P63, wherein X is S.

Embodiment P66. The compound, or a salt thereof, of any one of embodiments P1-P65, wherein R¹ comprises an oligonucleotide.

Embodiment P67. The compound, or a salt thereof, of embodiment P66, wherein the oligonucleotide is attached at its 5′ end.

Embodiment P68. The compound, or a salt thereof, of embodiment P66, wherein the oligonucleotide is attached at its 3′ end.

Embodiment P69. The compound, or a salt thereof, of embodiment P66, wherein the oligonucleotide is attached at an internal position on the oligonucleotide.

Embodiment P70. The compound, or a salt thereof, of embodiment P69, wherein the internal position is an internucleoside linkage.

Embodiment P71. The compound, or a salt thereof, of any one of embodiments P1-P70, wherein R¹ comprises an oligonucleotide conjugated to one or more additional CB₁ ligands.

Embodiment P72. The compound, or a salt thereof, of embodiment P71, wherein the oligonucleotide is conjugated to two, three, four, five, or more than five additional CB₁ ligands.

Embodiment P73. The compound, or a salt thereof, of embodiment P71 or P72, wherein the additional CB₁ ligands are conjugated to the oligonucleotide at the 5′ end of the oligonucleotide, the 3′ end of the oligonucleotide, one or more internal positions on the oligonucleotide, or any combination thereof.

Embodiment P74. The compound of any one of embodiments P66-P73, wherein the oligonucleotide is a modified oligonucleotide.

Embodiment P75. A compound comprising the structure of Formula (VI), or a salt thereof:

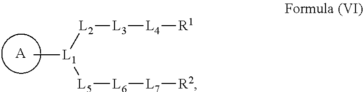

wherein

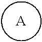

is a cannabinoid receptor type 1 (CB₁) ligand;
each of L₁, L₂, L₃, L₄, L₅, L₆, and L₇ is independently a linker, a bond, or absent; and
R¹ and R² each independently comprise one or more oligonucleotides, protecting groups, small molecules, proteins, antibodies, and/or peptides.

Embodiment P76. The compound, or a salt thereof, of embodiment P75, wherein the CB₁ ligand is a CB₁ agonist.

Embodiment P77. The compound, or a salt thereof, of embodiment P75, wherein the CB₁ ligand is a CB₁ antagonist.

Embodiment P78. The compound, or a salt thereof, of embodiment P75, wherein the CB₁ ligand is selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

Embodiment P79. The compound, or a salt thereof, of embodiment P75, wherein the CB₁ ligand comprises the structure

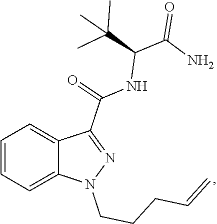 [SMILES: C=CCCCn1nc(C(=O)N[C@H](C(N)=O)C(C)(C)C)c2ccccc21]

or a derivative thereof.

Embodiment P80. The compound, or a salt thereof, of embodiment P75, wherein the compound comprises the structure of Formula (VII):

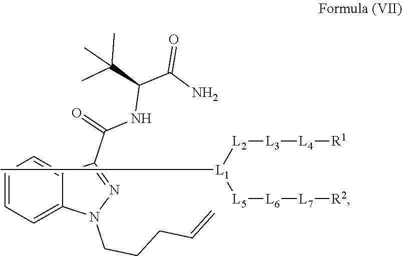

or a salt thereof.

Embodiment P81. The compound, or a salt thereof, of embodiment P80, wherein the compound comprises the structure of Formula (VII-a):

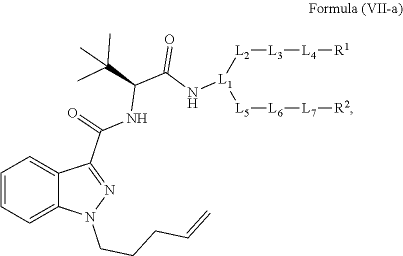

or a salt thereof.

Embodiment P82. The compound, or a salt thereof, of any one of embodiments P75-P81, wherein each of L₁, L₂, L₃, L₄, L₅, L₆, and L₇ is independently absent, a bond, an optionally substituted alkyl linker, an optionally substituted polyethylene glycol (PEG) linker, an optionally substituted heteroalkyl linker, an optionally substituted heteroaryl linker, a phosphodiester bond, or a phosphorothioate bond.

Embodiment P83. The compound, or a salt thereof, of embodiment P82, wherein L₁ is an optionally substituted PEG linker.

Embodiment P84. The compound, or a salt thereof, of embodiment P83, wherein L₁ is an optionally substituted PEG linker two or three PEG units in length.

Embodiment P85. The compound, or a salt thereof, of any one of embodiments P82-P84, wherein L₂ and L₅ are each independently an optionally substituted PEG linker.

Embodiment P86. The compound, or a salt thereof, of embodiment P85, wherein the L₂ and L₅ are each independently an optionally substituted PEG linker three or four PEG units in length.

Embodiment P87. The compound, or a salt thereof, of any one of embodiments P82-P86, wherein L₁, L₂, and L₅ together comprise the structure

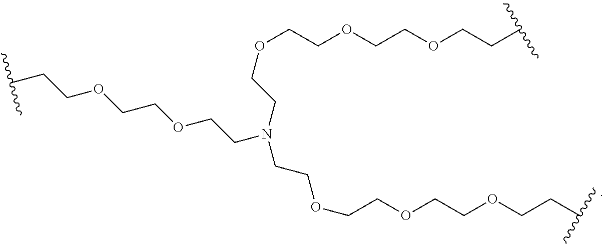 [SMILES: *CCOCCOCCOCCN(CCOCCOCC*)CCOCCOCCOCC*]

Embodiment P88. The compound, or a salt thereof, of any one of embodiments P82-P86, wherein L₃ and L₆ are each independently an optionally substituted heteroaryl linker.

Embodiment P89. The compound, or a salt thereof, of embodiment P88, wherein L₃ and L₆ are each independently an optionally substituted partially unsaturated heteroaryl linker.

Embodiment P90. The compound, or a salt thereof, of embodiment P88 or P89, wherein L₃ and L₆ each comprise the structure

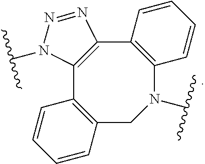 [SMILES: *N1Cc2ccccc2-c2c(nnn2*)-c2ccccc21]

Embodiment P91. The compound, or a salt thereof, of any one of embodiments P82-P90, wherein L₄ and L₇ are each independently an optionally substituted heteroalkyl linker.

Embodiment P92. The compound, or a salt thereof, of embodiment P91, wherein the heteroalkyl linker is substituted with one or more ═O substituents.

Embodiment P93. The compound, or a salt thereof, of embodiment P91 or P92, wherein L₄ and L₇ each comprise the structure

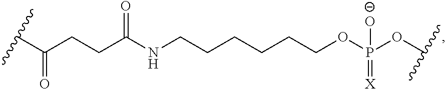

wherein X is O or S.

Embodiment P94. The compound, or a salt thereof, of any one of embodiments P82-P93, wherein L₁, L₂, L₃, L₄, L₅, L₆, and L₇ together comprise the structure

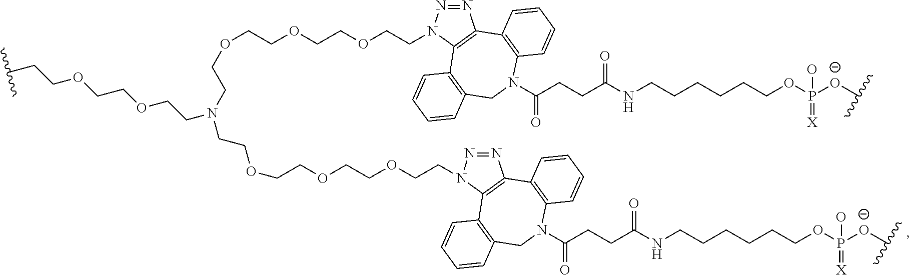

wherein X is O or S.

Embodiment P95. The compound, or a salt thereof, of any one of embodiments P75-P94, wherein the compound comprises the structure:

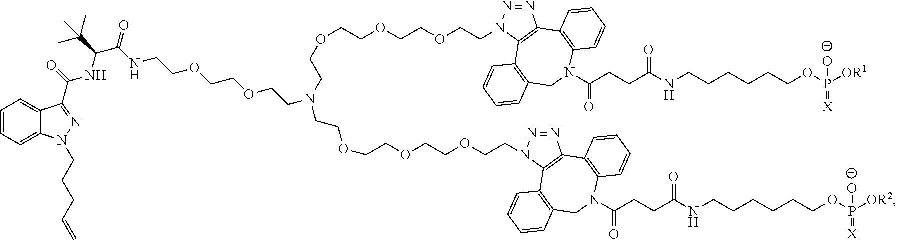

or a salt thereof, wherein X is O or S.

Embodiment P96. The compound, or a salt thereof, of any one of embodiments P75-P95, wherein X is O.

Embodiment P97. The compound, or a salt thereof, of any one of embodiments P75-P95, wherein X is S.

Embodiment P98. The compound, or a salt thereof, of any one of embodiments P75-P97, wherein R¹ and R² each comprise an oligonucleotide.

Embodiment P99. The compound, or a salt thereof, of embodiment P98, wherein one or both of the oligonucleotides is attached at its 5′ end.

Embodiment P100. The compound, or a salt thereof, of embodiment P98, wherein one or both of the oligonucleotides is attached at its 3′ end.

Embodiment P101. The compound, or a salt thereof, of embodiment P98, wherein one or both of the oligonucleotides is attached at an internal position on the oligonucleotide.

Embodiment P102. The compound, or a salt thereof, of embodiment P101, wherein the internal position is an internucleoside linkage.

Embodiment P103. The compound, or a salt thereof, of any one of embodiments P98-P102, wherein R¹ and R² each comprise an oligonucleotide conjugated to one or more additional CB₁ ligands.

Embodiment P104. The compound, or a salt thereof, of embodiment P103, wherein the oligonucleotide is conjugated to two, three, four, five, or more than five additional CB₁ ligands.

Embodiment P105. The compound, or a salt thereof, of embodiment P103 or P104, wherein the additional CB₁ ligands are conjugated to the oligonucleotide at the 5′ end of the oligonucleotide, the 3′ end of the oligonucleotide, one or more internal positions on the oligonucleotide, or any combination thereof.

Embodiment P106. The compound of any one of embodiments P98-P105, wherein the oligonucleotide is a modified oligonucleotide.

Embodiment P107. A composition comprising a compound, or a salt thereof, of any one of embodiments P1-P106, and a pharmaceutically acceptable excipient.

Embodiment P108. A method for delivering a therapeutic oligonucleotide to the brain of a subject, comprising administration of a compound, or a salt thereof, of any one of embodiments P1-P106, or a composition of embodiment P107, to the subject.

Embodiment P109. The method of embodiment P108, wherein the therapeutic oligonucleotide is delivered to one or more brain regions selected from the group consisting of the striatum, the cerebellum, the brain stem, the hippocampus, the frontal cortex, and the spinal cord.

Embodiment P110. A method for treating or ameliorating a disease, disorder, or symptom thereof in a subject, comprising administration of a compound, or a salt thereof, of any one of embodiments P1-P106, or a composition of embodiment P107, to the subject.

Embodiment P111. The method of embodiment P110, wherein the disease, disorder, or symptom thereof is a central nervous system (CNS) disease, disorder, or symptom thereof.

Embodiment P112. The method of embodiment P110 or P111, wherein the disease, disorder, or symptom thereof is Alzheimer's disease, or a symptom thereof.

Embodiment P113. The method of any one of embodiments P108-P112, wherein the compound, or a salt thereof, is administered to the subject intrathecally.

Embodiment P114. A method for making a compound, or a salt thereof, of any one of embodiments P1-P106, comprising one or more compounds and chemical transformations described herein, including Example 1.

Additional embodiments include embodiment 1 to embodiment 166 following.

Embodiment 1. A compound, or a stereoisomer, tautomer, prodrug, or salt thereof, comprising the structure of Formula (I′):

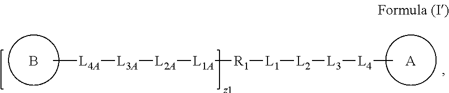

wherein:
each of

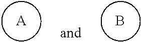

is independently a cannabinoid receptor type 1 (CB₁) ligand;
each of L₁, L₂, L₃, L₄, L_{1A}, L_{2A}, L_{3A}, and L_{4A} is independently a linker, a bond, or absent;
R¹ comprises one or more oligonucleotides, protecting groups, small molecules, proteins, antibodies, and/or peptides; and
z1 is 0 or 1.

Embodiment 2. The compound, or a stereoisomer, tautomer, prodrug, or salt thereof, of embodiment 1, wherein the compound comprises the structure of Formula (I″):

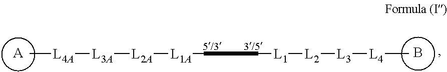

wherein:
[symbol] is an oligonucleotide.

Embodiment 3. The compound, or a stereoisomer, tautomer, prodrug, or salt thereof, of embodiment 2, wherein the compound comprises the structure of Formula (I′-a):

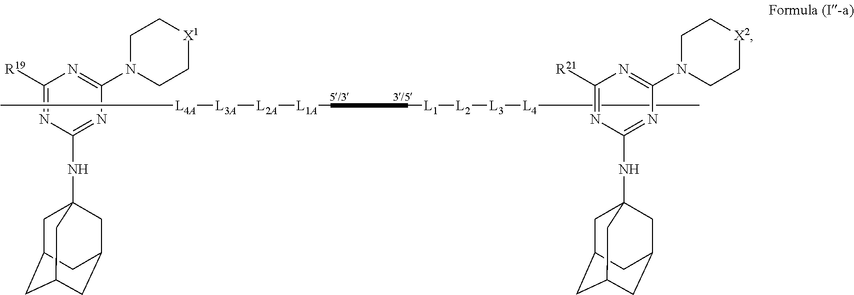

wherein:
X¹ is NR¹⁰ or CR¹¹R¹²;
X² is NR¹³ or CR¹⁴R¹⁵;
R¹⁰, R, R¹², R¹³, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D};
—NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, —SOₙ₂₁R^{21A}, —SOᵥ₂₁NR^{21B}R^{21C}, —NHNR^{21B}R^{21C}, ONR^{21B}R^{21C}, —NHC(O)NHNR^{21B}R^{21C}, —NHC(O)NR^{21B}R^{21C}, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{2D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D}, R^{21A}, R^{21B}, R^{21C} and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C}; and R^{21B} and R^{21C} substituents bonded to the same nitrogen atom may optionally be joined to form a substituted or unsubstituted heterocycloalkyl or substituted or unsubstituted heteroaryl;
n19 and n21 are each independently 0, 1, 2, 3, or 4; and
v19 and v21 are each independently 1 or 2.

Embodiment 4. The compound, or a stereoisomer, tautomer, prodrug, or salt thereof, of embodiment 3, wherein the compound comprises the structure of Formula (I″-a-1):

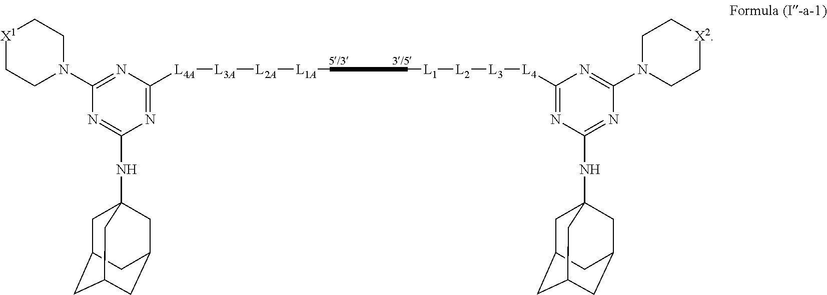

Embodiment 5. The compound, or a stereoisomer, tautomer, prodrug, or salt thereof, of embodiment 3, wherein the compound comprises the structure of Formula (I″-a-2):

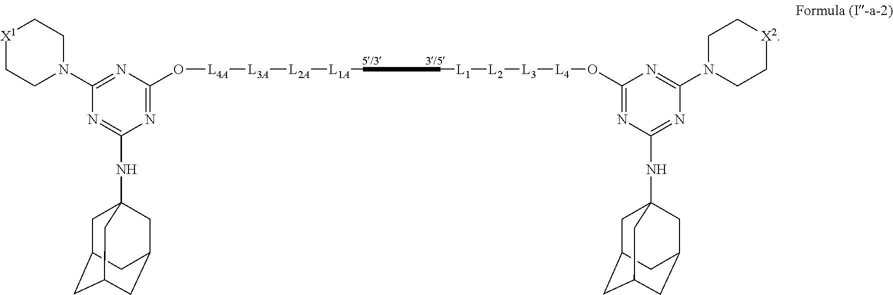

Embodiment 6. The compound, or a stereoisomer, tautomer, prodrug, or salt thereof, of embodiment 2, wherein the compound comprises the structure of Formula (I′-b):

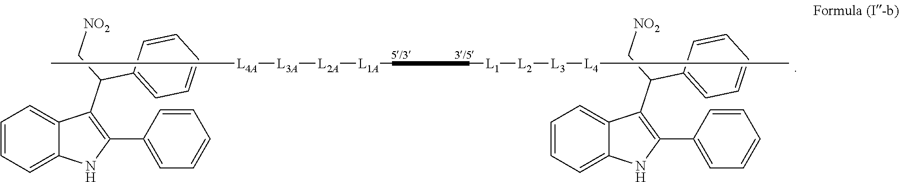

Embodiment 7. The compound, or a stereoisomer, tautomer, prodrug, or salt thereof, of embodiment 6, wherein the compound comprises the structure of Formula (I′-b-1):

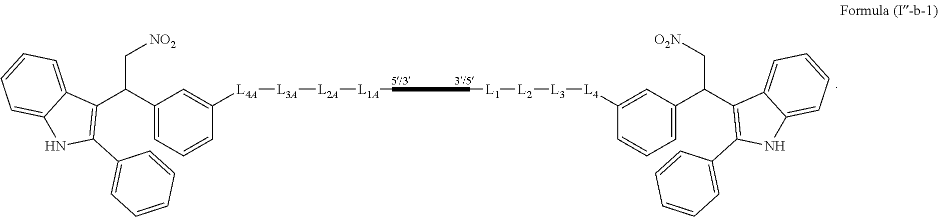

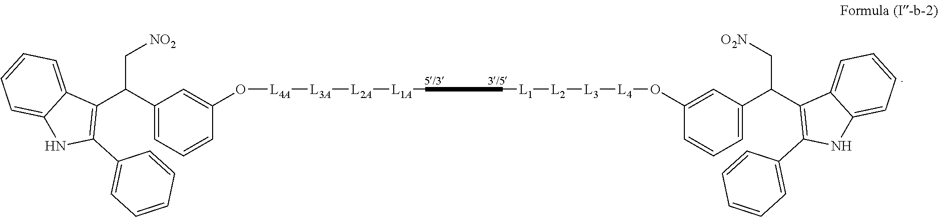

Embodiment 9. The compound, or a stereoisomer, tautomer, prodrug, or salt thereof, of embodiment 2, wherein the compound comprises the structure of Formula (I″-c):

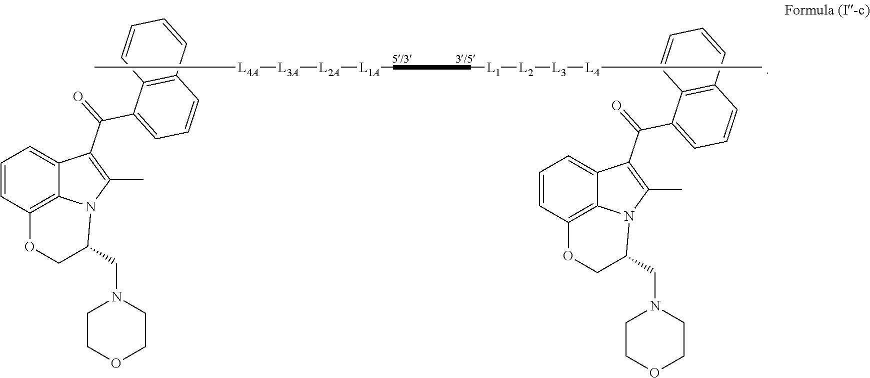

Embodiment 10. The compound, or a stereoisomer, tautomer, prodrug, or salt thereof, of embodiment 9, wherein the compound comprises the structure of Formula (I″-c-1):

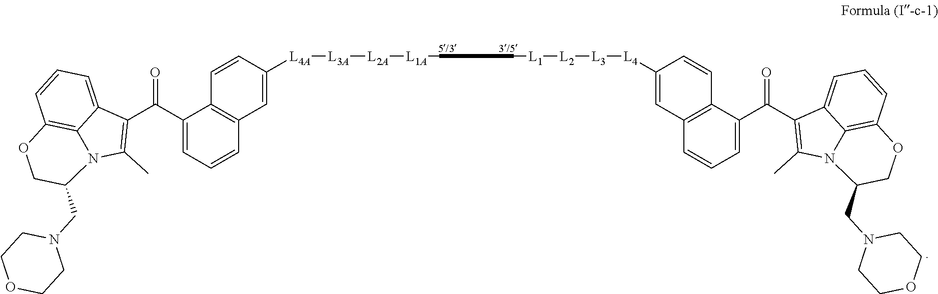

Embodiment 11. The compound, or a stereoisomer, tautomer, prodrug, or salt thereof, of embodiment 9, wherein the compound comprises the structure of Formula (I″-c-2):

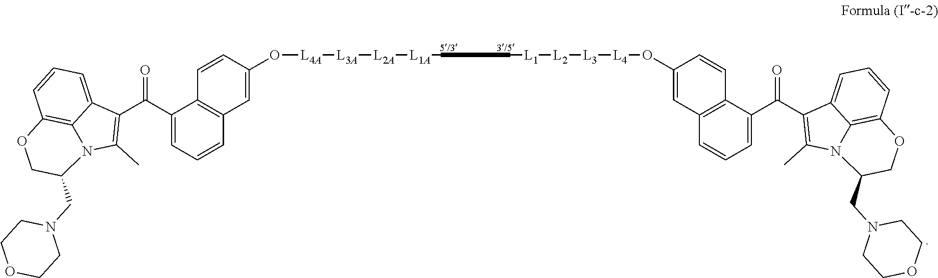

Embodiment 12. A compound, or a stereoisomer, tautomer, prodrug, or salt thereof, comprising the structure of Formula (I):

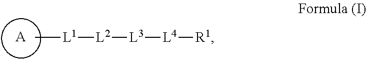

wherein

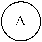

is a cannabinoid receptor type 1 (CB₁) ligand;
each of L₁, L₂, L₃, and L₄ is independently a linker, a bond, or absent; and
R¹ comprises one or more oligonucleotides, protecting groups, small molecules, proteins, antibodies, and/or peptides.

Embodiment 13. The compound, or a stereoisomer, tautomer, prodrug, or salt thereof, of embodiment 12, wherein the CB₁ ligand is a CB₁ agonist.

Embodiment 14. The compound, or a stereoisomer, tautomer, prodrug, or salt thereof, of embodiment 12, wherein the CB₁ ligand is a CB₁ antagonist.

Embodiment 15. The compound, or a stereoisomer, tautomer, prodrug, or salt thereof, of embodiment 12, wherein the CB₁ ligand is a selective ligand.

Embodiment 16. The compound, or a stereoisomer, tautomer, prodrug, or salt thereof, of embodiment 12, wherein the CB₁ ligand is a non-selective ligand.

Embodiment 17. The compound, or a stereoisomer, tautomer, prodrug, or salt thereof, of any one of embodiments 12-16, wherein the CB₁ ligand is selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

Embodiment 18. The compound, or a stereoisomer, tautomer, prodrug, or salt thereof, of embodiment 12, wherein the CB₁ ligand comprises the structure of Formula (II′):

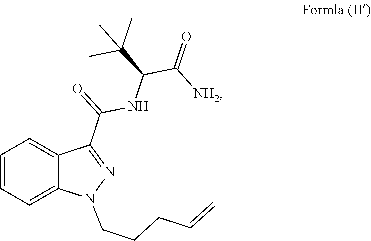

wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D};
—NR^{17B}C(O)OR^{7D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, and R^{17D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl;
n17 is 0, 1, 2, 3, or 4; and
v17 is 1 or 2.

Embodiment 19. The compound, or a stereoisomer, tautomer, prodrug, or salt thereof, of embodiment 18, wherein R¹⁷ is —NR^{17B}R^{17C}, —C(O)R^{17D}, or —C(O)OR^{17D}.

Embodiment 20. The compound, or a stereoisomer, tautomer, prodrug, or salt thereof, of embodiment 19, wherein R^{17B} and R^{17C} are each independently hydrogen, optionally substituted alkyl, or optionally substituted heteroalkyl.

Embodiment 21. The compound, or a stereoisomer, tautomer, prodrug, or salt thereof, of embodiment 20, wherein the CB₁ ligand comprises the structure

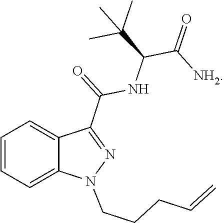 [SMILES: C=CCCCn1nc(C(=O)N[C@H](C(N)=O)C(C)(C)C)c2ccccc21]

Embodiment 22. The compound, or a stereoisomer, tautomer, prodrug, or salt thereof, of embodiment 12, wherein the compound comprises the structure of Formula (II):

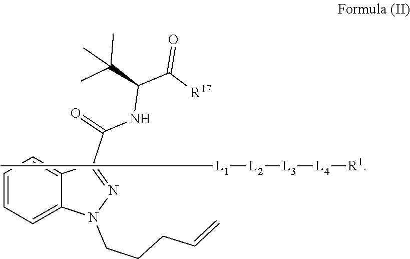

Embodiment 23. The compound, or a stereoisomer, tautomer, prodrug, or salt thereof, of embodiment 22, wherein the compound comprises the structure of Formula (II-a):

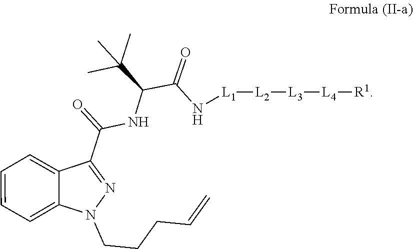

Embodiment 24. The compound, or a stereoisomer, tautomer, prodrug, or salt thereof, of embodiment 12, wherein the CB₁ ligand comprises the structure

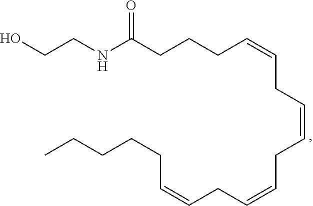 [SMILES: CCCCC/C=C\C/C=C\C/C=C\C/C=C\CCCC(=O)NCCO]

or a derivative thereof.

Embodiment 25. The compound, or a stereoisomer, tautomer, prodrug, or salt thereof, of embodiment 12, wherein the compound comprises the structure of Formula (III):

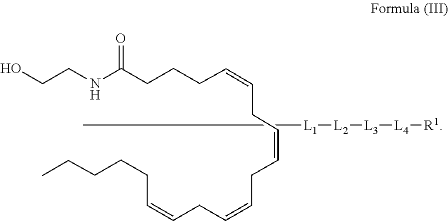

Embodiment 26. The compound, or a stereoisomer, tautomer, prodrug, or salt thereof, of embodiment 25, wherein the compound comprises the structure of Formula (III-a):

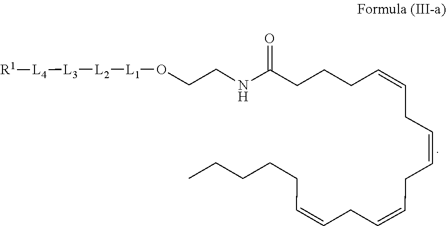

Embodiment 27. A compound, or a stereoisomer, tautomer, prodrug, or salt thereof, comprising the structure of Formula (VIII):

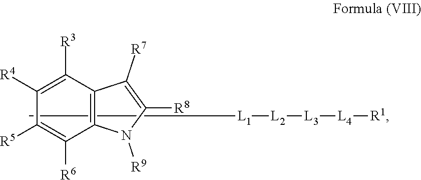

wherein:
each of L₁, L₂, L₃, and L₄ is independently a linker, a bond, or absent;
R¹ comprises one or more oligonucleotides, protecting groups, small molecules, proteins, antibodies, and/or peptides;
R³, R⁴, R⁵, R⁶, and R⁸ are each independently hydrogen, halogen, optionally substituted alkyl, optionally substituted heteroalkyl, optionally substituted cycloalkyl, optionally substituted heterocycloalkyl, optionally substituted aryl, or optionally substituted heteroaryl; and
R⁹ is hydrogen, optionally substituted alkyl, or optionally substituted heteroalkyl; or R⁶ and R⁹ substituents may be joined together form an optionally substituted heterocycloalkyl or optionally substituted heteroaryl;
R⁷ is hydrogen, —SOᵥ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D};
—NR^{7B}C(O)OR^{7D}, —NR^{7B}OR^{7D}, optionally substituted alkyl, optionally substituted heteroalkyl, optionally substituted cycloalkyl, optionally substituted heterocycloalkyl, optionally substituted aryl, or optionally substituted heteroaryl;
R^{7A}, R^{7B}, R^{7C}, R^{7D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{7B} and R^{7C} substituents bonded to the same nitrogen atom may optionally be joined to form a substituted or unsubstituted heterocycloalkyl or substituted or unsubstituted heteroaryl;
n7 is 0, 1, 2, 3, or 4; and
v7 is 1 or 2.

Embodiment 28. The compound, or a stereoisomer, tautomer, prodrug, or salt thereof, of embodiment 27, wherein R⁷ and R⁸ are each independently hydrogen, optionally substituted alkyl, or optionally substituted heteroalkyl.

Embodiment 29. The compound, or a stereoisomer, tautomer, prodrug, or salt thereof, of embodiment 27 or 28, wherein:
R⁴ is halogen; and
R³, R⁵, and R⁶ are each independently hydrogen.

Embodiment 30. The compound, or a stereoisomer, tautomer, prodrug, or salt thereof, of embodiment 27 or 28, wherein R³, R⁴, R⁵, and R⁶ are each independently hydrogen.

Embodiment 31. The compound, or a stereoisomer, tautomer, prodrug, or salt thereof, of embodiment 27 or 28, wherein R⁶ and R⁹ substituents are joined together to form an optionally substituted heterocycloalkyl or optionally substituted heteroaryl.

Embodiment 32. The compound, or a stereoisomer, tautomer, prodrug, or salt thereof, of embodiment 27 or 28, wherein R⁹ is hydrogen or optionally substituted alkyl.

Embodiment 33. The compound, or a stereoisomer, tautomer, prodrug, or salt thereof, of embodiment 27, wherein the compound comprises the structure of Formula (VIII-a):

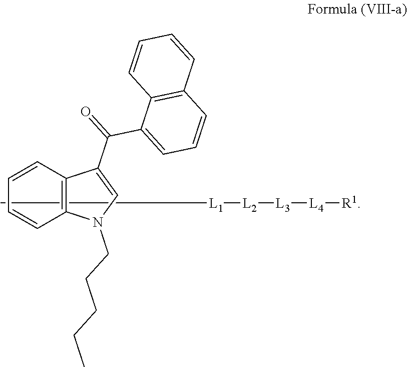

Embodiment 34. The compound, or a stereoisomer, tautomer, prodrug, or salt thereof, of embodiment 33, wherein the compound comprises the structure of Formula (VIII-a-1):

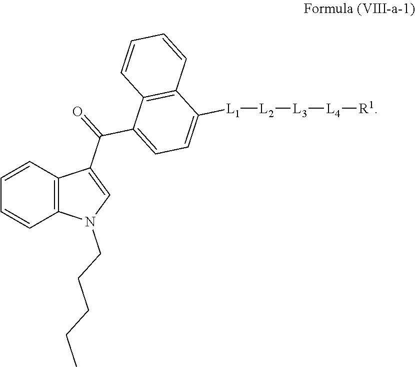

Embodiment 35. The compound, or a stereoisomer, tautomer, prodrug, or salt thereof, of embodiment 27, wherein the compound comprises the structure of Formula (VIII-a-2):

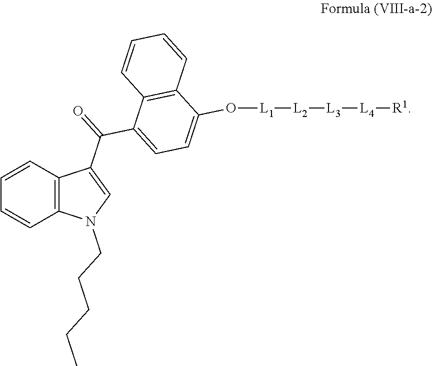

Embodiment 36. The compound, or a stereoisomer, tautomer, prodrug, or salt thereof, of embodiment 27, wherein the compound comprises the structure of Formula (VIII-b):

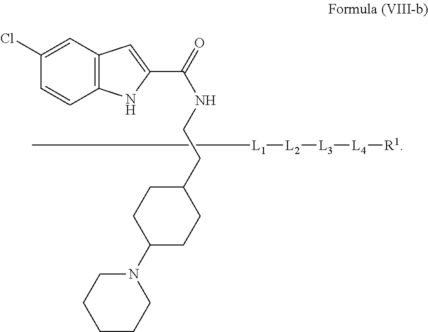

Embodiment 37. The compound, or a stereoisomer, tautomer, prodrug, or salt thereof, of embodiment 27 or 36, wherein the compound comprises the structure of Formula (VIII-b-1):

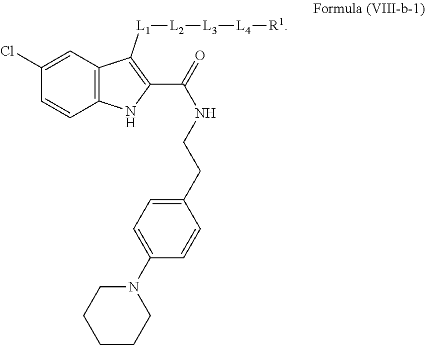

Embodiment 38. The compound, or a stereoisomer, tautomer, prodrug, or salt thereof, of embodiment 27, wherein the compound comprises the structure of Formula (VIII-c):

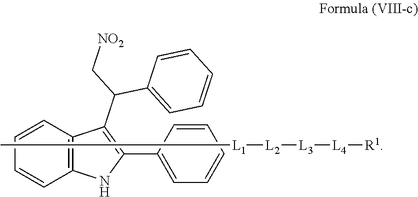

Embodiment 39. The compound, or a stereoisomer, tautomer, prodrug, or salt thereof, of embodiment 27 or 38, wherein the compound comprises the structure of Formula (VIII-c-1):

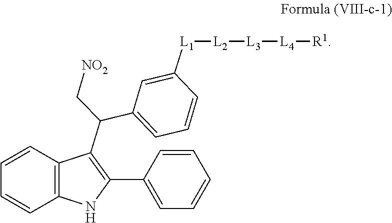

Embodiment 40. The compound, or a stereoisomer, tautomer, prodrug, or salt thereof, of embodiment 27, wherein the compound comprises the structure of Formula (VIII-c-2):

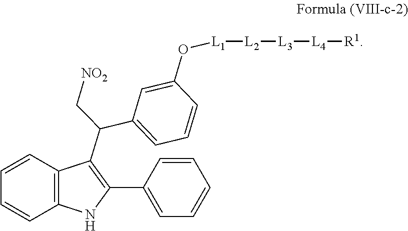

Embodiment 41. The compound, or a stereoisomer, tautomer, prodrug, or salt thereof, of embodiment 27, wherein the compound comprises the structure of Formula (VIII-d):

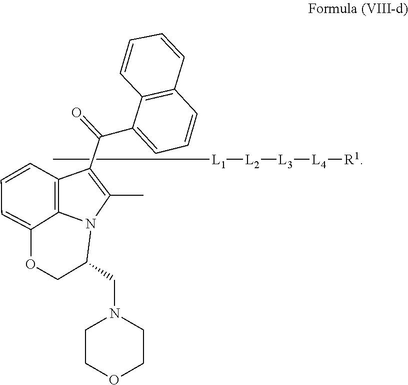

Embodiment 42. The compound, or a stereoisomer, tautomer, prodrug, or salt thereof, of embodiment 27 or 41, wherein the compound comprises the structure of Formula (VIII-d-1):

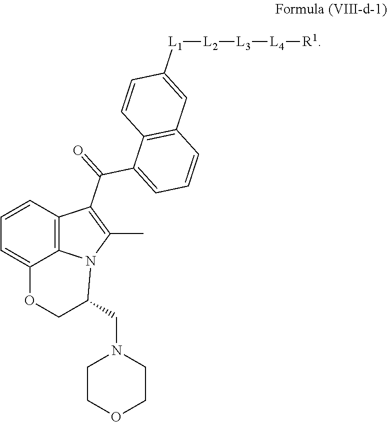

Embodiment 43. The compound, or a stereoisomer, tautomer, prodrug, or salt thereof, of embodiment 27, wherein the compound comprises the structure of Formula (VIII-d-2):

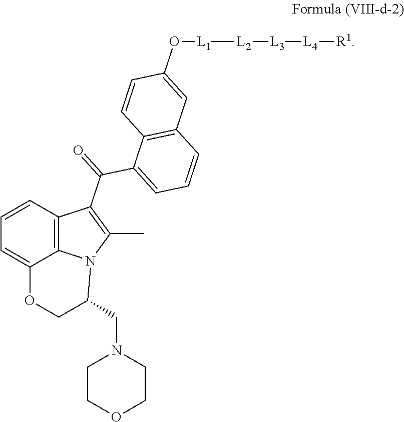

Embodiment 44. The compound, or a stereoisomer, tautomer, prodrug, or salt thereof, of embodiment 12, comprising the structure of Formula (IX):

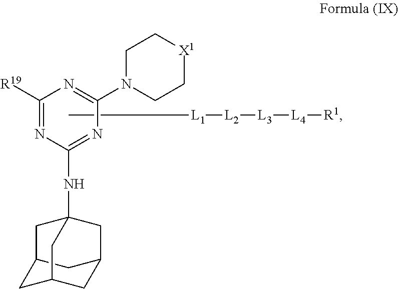

wherein:
each of L₁, L₂, L₃, and L₄ is independently a linker, a bond, or absent;
R¹ comprises one or more oligonucleotides, protecting groups, small molecules, proteins, antibodies, and/or peptides;
X¹ is NR¹⁰ or CR¹¹R¹²;
R¹⁰, R¹¹, and R¹² are each independently hydrogen, optionally substituted alkyl, optionally substituted heteroalkyl, optionally substituted cycloalkyl, optionally substituted heterocycloalkyl, optionally substituted aryl, or optionally substituted heteroaryl; and
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D};
NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C} substituents bonded to the same nitrogen atom may optionally be joined to form a substituted or unsubstituted heterocycloalkyl or substituted or unsubstituted heteroaryl;
n19 is 0, 1, 2, 3, or 4; and
v19 is 1 or 2.

Embodiment 45. The compound, or a stereoisomer, tautomer, prodrug, or salt thereof, of embodiment 12 or 44, wherein the compound comprises the structure of Formula (IX-a-1):

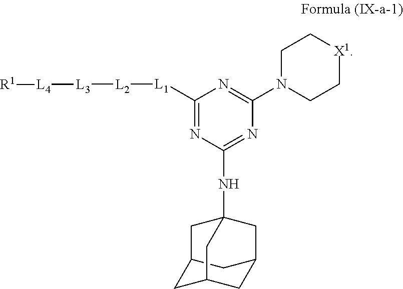

Embodiment 46. The compound, or a stereoisomer, tautomer, prodrug, or salt thereof, of embodiment 12 or 44, wherein the compound comprises the structure of Formula (IX-a-2):

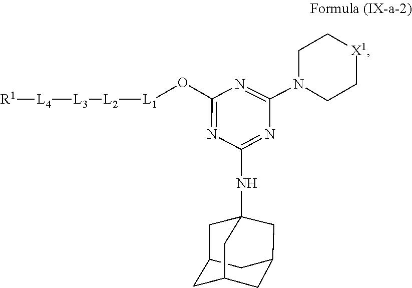

Embodiment 47. The compound, or a stereoisomer, tautomer, prodrug, or salt thereof, of any one of embodiments 44-46, wherein:
X¹ is NR¹⁰; and
R¹⁰ is hydrogen or optionally substituted alkyl.

Embodiment 48. The compound, or a stereoisomer, tautomer, prodrug, or salt thereof, of embodiment 47, wherein:
R¹⁰ is hydrogen, —CH₃, or —CH₂CH₂F.

Embodiment 49. The compound, or a stereoisomer, tautomer, prodrug, or salt thereof, of any one of embodiments 44-46, wherein:
X¹ is CR¹¹R¹²; and
R¹¹ and R¹² are each independently hydrogen or optionally substituted alkyl.

Embodiment 50. The compound, or a stereoisomer, tautomer, prodrug, or salt thereof, of embodiment 49, wherein:
R¹¹ is hydrogen, —CH₃, or —CH₂CH₂F; and
R¹² is hydrogen.

Embodiment 51. The compound, or a stereoisomer, tautomer, prodrug, or salt thereof, of embodiment 12, comprising the structure of Formula (X):

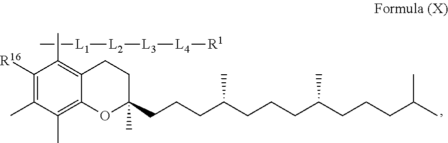

wherein:
each of L₁, L₂, L₃, and L₄ is independently a linker, a bond, or absent;
R¹ comprises one or more oligonucleotides, protecting groups, small molecules, proteins, antibodies, and/or peptides;
R¹⁶ is hydrogen, halogen, —CN, —N₃, —NO₂, —NR^{6B}R^{16C}, —C(O)R^{6D}, —C(O)OR^{16D}, —C(O)NR^{16B}R^{16C}, —OR^{16A}, —NR^{16B}C(O)R^{16D}, optionally substituted alkyl, optionally substituted heteroalkyl, optionally substituted cycloalkyl, optionally substituted heterocycloalkyl, optionally substituted aryl, or optionally substituted heteroaryl; and
R^{16A}, R^{16B}, R^{16C}, and R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{16B} and R^{16C} substituents bonded to the same nitrogen atom may optionally be joined to form a substituted or unsubstituted heterocycloalkyl or substituted or unsubstituted heteroaryl.

Embodiment 52. The compound, or a stereoisomer, tautomer, prodrug, or salt thereof, of embodiment 51, wherein the compound comprises the structure of Formula (X-a):

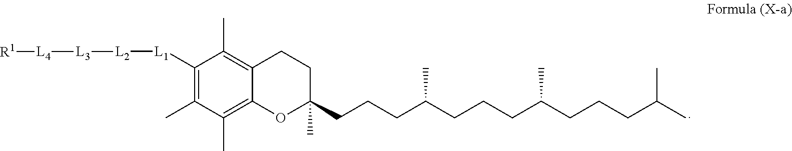

Embodiment 53. The compound, or a stereoisomer, tautomer, prodrug, or salt thereof, of embodiment 12 or 52, wherein the compound comprises the structure of Formula (X-a-1):

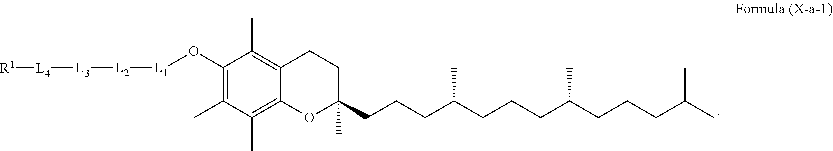

Embodiment 54. The compound, or a stereoisomer, tautomer, prodrug, or salt thereof, of embodiment 12, wherein the CB₁ ligand comprises the structure:

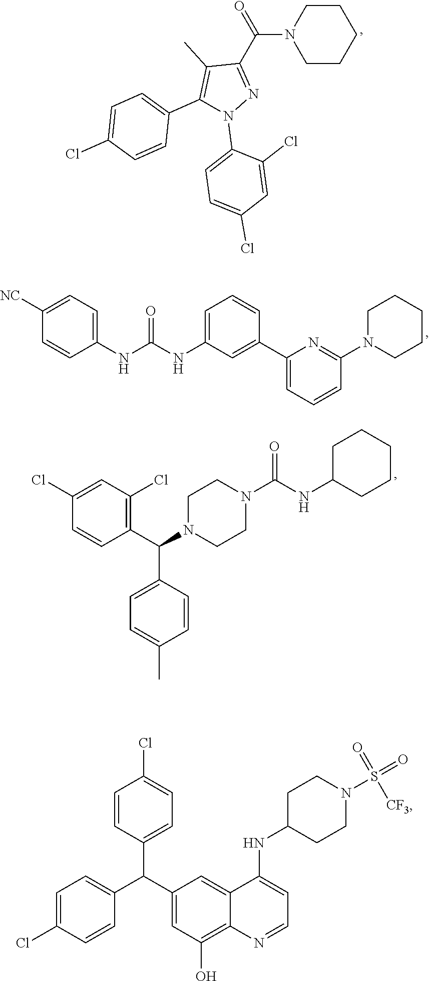

or a derivative thereof.

Embodiment 55. The compound, or a stereoisomer, tautomer, prodrug, or salt thereof, of embodiment 12, wherein the compound comprises the structure:

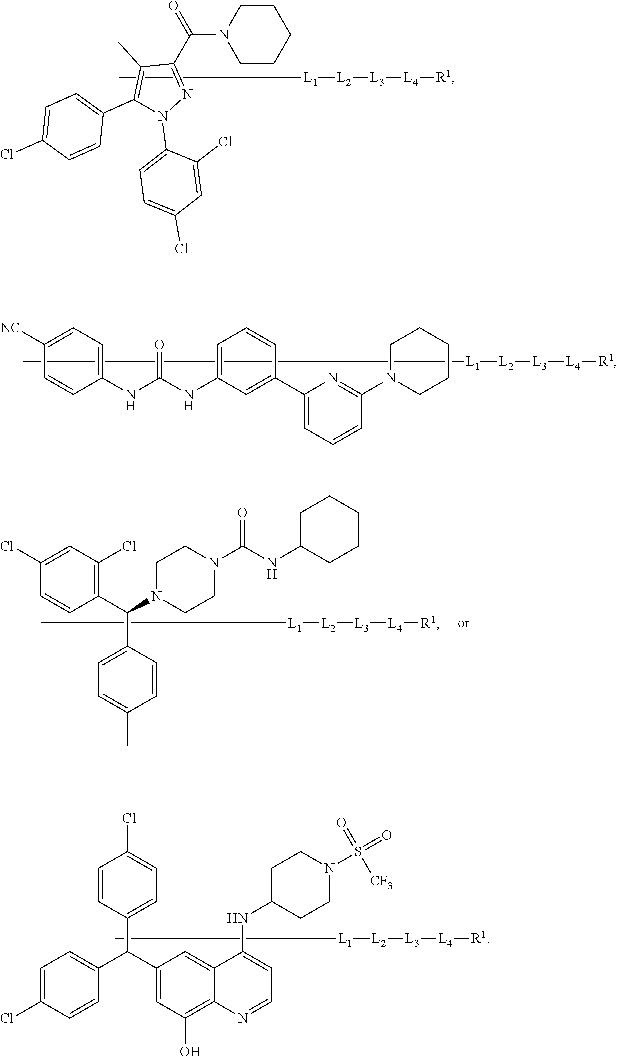

Embodiment 56. The compound, or a stereoisomer, tautomer, prodrug, or salt thereof, of any one of embodiments 1-55, wherein each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, oxygen, optionally substituted nitrogen, an amide, a phosphodiester bond, or a phosphorothioate bond.

Embodiment 57. The compound, or a stereoisomer, tautomer, prodrug, or salt thereof, of any one of embodiments 1-56, wherein L₁ is a bond.

Embodiment 58. The compound, or a stereoisomer, tautomer, prodrug, or salt thereof, of any one of embodiments 1-56, wherein L₁ is oxygen.

Embodiment 59. The compound, or a stereoisomer, tautomer, prodrug, or salt thereof, of any one of embodiments 1-56, wherein L₁ comprises the structure

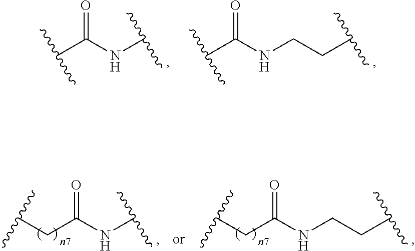

wherein n7 is 0, 1, 2, 3, 4, 5, 6, 7, 8, 9, or 10.

Embodiment 60. The compound, or a stereoisomer, tautomer, prodrug, or salt thereof, of any one of embodiments 1-59, wherein L₂ is an optionally substituted PEG linker.

Embodiment 61. The compound, or a stereoisomer, tautomer, prodrug, or salt thereof, of embodiment 60, wherein the PEG linker is 1, 2, 3, 4, 5, 6, 7, 8, 9, or 10 PEG units in length.

Embodiment 62. The compound, or a stereoisomer, tautomer, prodrug, or salt thereof, of any one of embodiments 1-59, wherein L₂ is an optionally substituted alkyl linker.

Embodiment 63. The compound, or a stereoisomer, tautomer, prodrug, or salt thereof, of embodiment 62, wherein L₂ comprises the structure

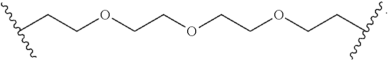 [SMILES: *CCOCCOCCOCC*]

Embodiment 64. The compound, or a stereoisomer, tautomer, prodrug, or salt thereof, of any one of embodiments 1-63, wherein L₄ is an optionally substituted heteroalkyl linker or a bond.

Embodiment 65. The compound, or a stereoisomer, tautomer, prodrug, or salt thereof, of embodiment 64, wherein the heteroalkyl linker is substituted with one or more ═O substituents.

Embodiment 66. The compound, or a stereoisomer, tautomer, prodrug, or salt thereof, of embodiment 64 or 65, wherein L₄ comprises the structure

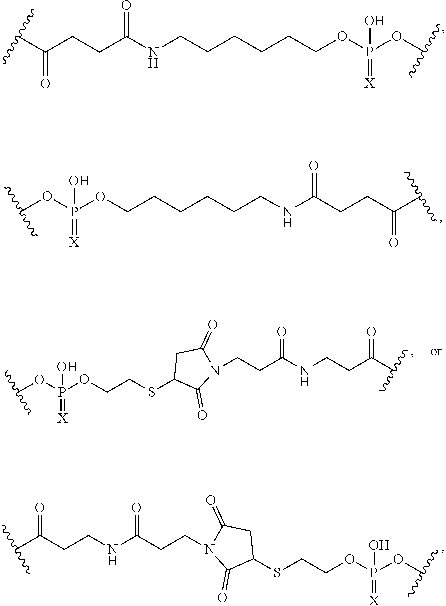

wherein X is O or S.

Embodiment 67. The compound, or a stereoisomer, tautomer, prodrug, or salt thereof, of embodiment 66, wherein L₄ comprises the structure

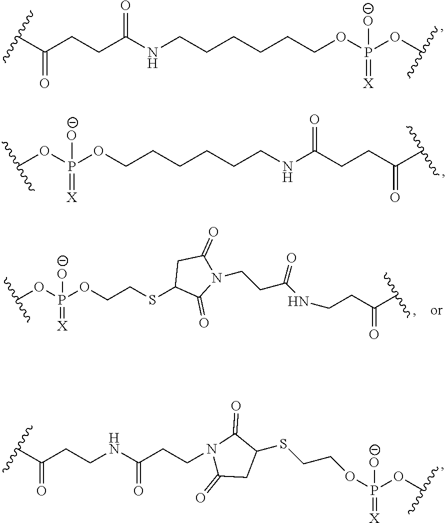

wherein X is O or S.

Embodiment 68. The compound, or a stereoisomer, tautomer, prodrug, or salt thereof, of any one of embodiments 1-67, wherein L₃ is an optionally substituted heteroaryl linker.

Embodiment 69. The compound, or a stereoisomer, tautomer, prodrug, or salt thereof, of any one of embodiments 1-63, wherein one of L₃ and L₄ is an optionally substituted phosphodiester bond or an optionally substituted phosphorothioate bond, and the other of L₃ and L₄ is a bond.

Embodiment 70. The compound, or a stereoisomer, tautomer, prodrug, or salt thereof, of any one of embodiments 1-67, wherein L₃ is an optionally substituted partially unsaturated heteroaryl or optionally substituted partially unsaturated heterocycloalkyl linker.

Embodiment 71. The compound, or a stereoisomer, tautomer, prodrug, or salt thereof, of embodiment 70, wherein L₃ comprises the structure

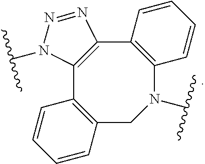 [SMILES: *N1Cc2ccccc2-c2c(nnn2*)-c2ccccc21]

Embodiment 72. The compound, or a stereoisomer, tautomer, prodrug, or salt thereof, of any one of embodiments 1-71, wherein L₁, L₂, L₃, and L₄ together comprise the structure:

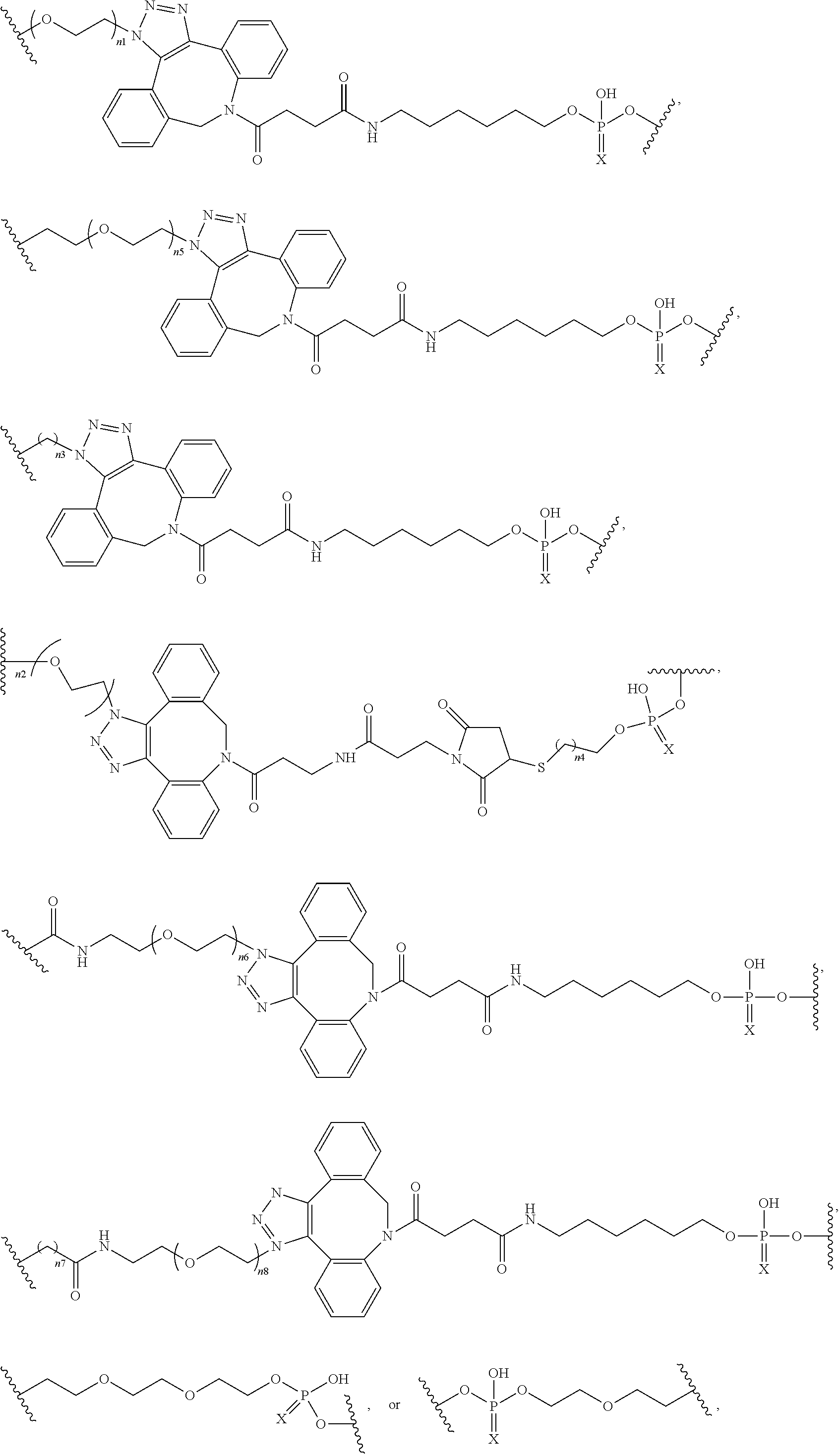

wherein:
X is O or S; and
n1, n2, n4, n5, n6, n7, and n8 are each independently 0, 1, 2, 3, 4, 5, 6, 7, 8, 9, or 10; and
n3 is 0, 1, 2, 3, 4, 5, 6, 7, 8, 9, 10, 11, 12, 13, 14, 15, 16, 17, 18, 19, 20, 21, or 22.

Embodiment 73. The compound, or a stereoisomer, tautomer, prodrug, or salt thereof, of embodiment 72, wherein L₁, L₂, L₃, and L₄ together comprise the structure:

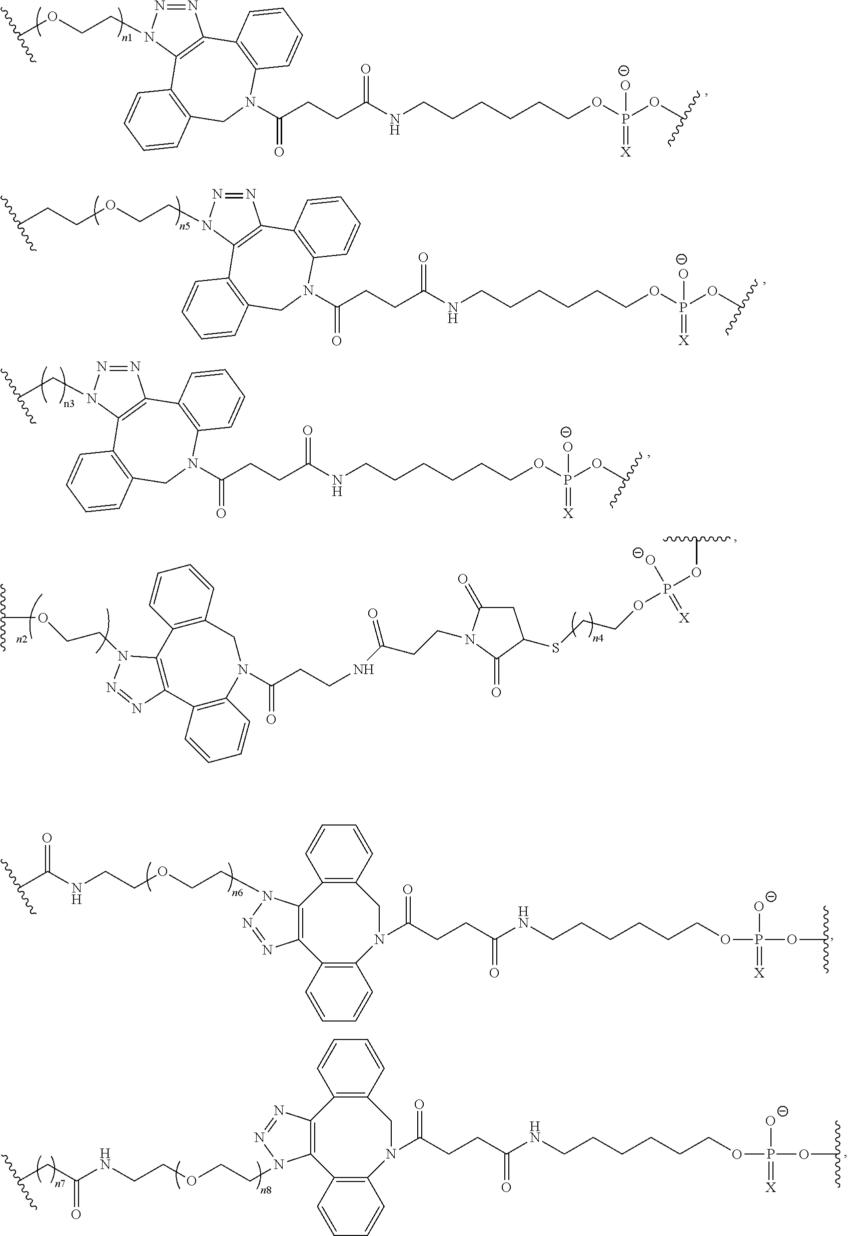

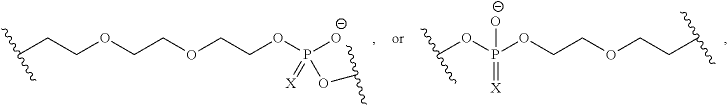

wherein:
X is O or S;
n1, n2, n4, n5, n6, n7, and n8 are each independently 0, 1, 2, 3, 4, 5, 6, 7, 8, 9, or 10; and
n3 is 0, 1, 2, 3, 4, 5, 6, 7, 8, 9, 10, 11, 12, 13, 14, 15, 16, 17, 18, 19, 20, 21, or 22.

Embodiment 74. The compound, or a stereoisomer, tautomer, prodrug, or salt thereof, of embodiment 66, 67, 72, or 73, wherein X is S.

Embodiment 75. The compound, or a stereoisomer, tautomer, prodrug, or salt thereof, of embodiment 66, 67, 72, or 73, wherein X is O.

Embodiment 76. A compound, or a stereoisomer, tautomer, prodrug, or salt thereof, comprising the structure of Formula (IV′), or a salt thereof:

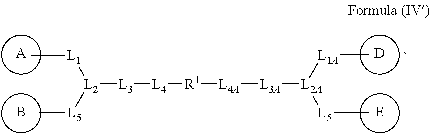

wherein:

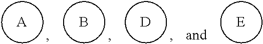

and are each independently a cannabinoid receptor type 1 (CB₁) ligand, or one of

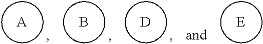

is a CB₁ ligand, and the other of

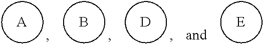

comprises a lipid or a ligand;
each of L₁, L₂, L₃, L₄, L₅, L_{1A}, L_{2A}, L_{A3}, L_{A4}, and L_{A5} are each independently a linker, a bond, or absent; and
R¹ comprises one or more oligonucleotides, protecting groups, small molecules, proteins, antibodies, and/or peptides.

Embodiment 77. The compound, or a stereoisomer, tautomer, prodrug, or salt thereof, of embodiment 76, wherein the compound comprises the structure of Formula (IV″):

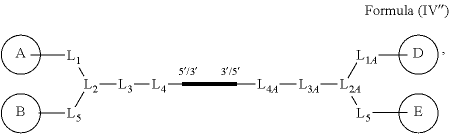

wherein:
[symbol] is an oligonucleotide.

Embodiment 78. The compound, or a stereoisomer, tautomer, prodrug, or salt thereof, of embodiment 77, wherein the compound comprises the structure of Formula (IV″-a):

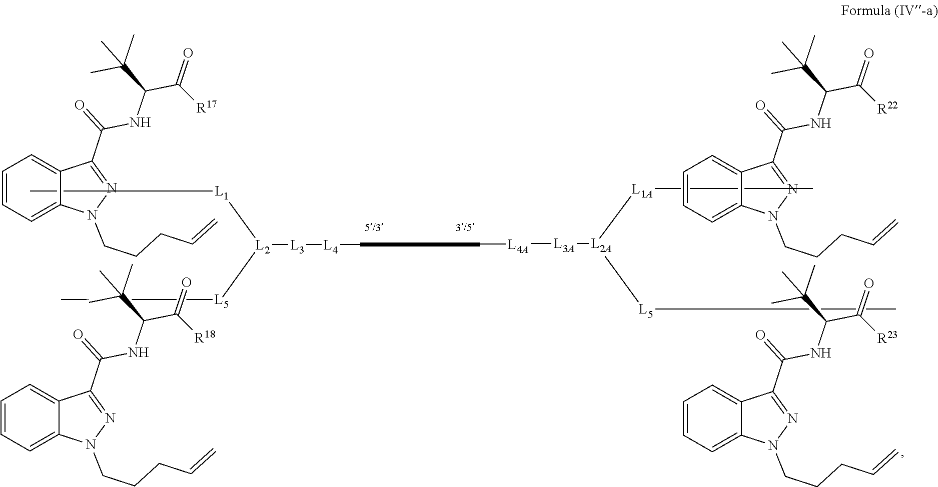

wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D};
—NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R^{18C}, —NHNR^{18B}R^{18C}, —ONR^{18B}R^{18C}, —NHC(O)NHNR^{18B}R^{18C}, —NHC(O)NR^{18B}R^{18C}, —NR^{18B}R^{18C}, —C(O)R^{18D}, —C(O)OR^{18D}, —C(O)NR^{18B}R^{18C}, —OR^{18A}, —NR^{18B}SO₂R^{18A}, —NR^{18B}C(O)R^{18D}, —NR^{18B}C(O)OR^{18D}, —NR^{18B}OR^{18D}, optionally substituted alkyl, optionally substituted heteroalkyl, optionally substituted cycloalkyl, optionally substituted heterocycloalkyl, optionally substituted aryl, or optionally substituted heteroaryl; R²² is hydrogen, —SOₙ₂₂R^{22A}, —SOᵥ₂₂NR^{22B}R^{22C}, —NHNR^{22B}R^{22C}, —ONR^{22B}R^{22C}, —NHC(O)NHNR^{22B}R^{22C}, —NHC(O)NR^{22B}R^{22C}, —NR^{22B}R^{22C}, —C(O)R^{22D}, —C(O)OR^{22D}, —C(O)NR^{22B}R^{22C}, —OR^{22A}, —NR^{22B}SO₂R^{22A}, —NR^{22B}C(O)R^{22D};
—NR^{22B}C(O)OR^{22D}, —NR^{22B}OR^{22D}, optionally substituted alkyl, optionally substituted heteroalkyl, optionally substituted cycloalkyl, optionally substituted heterocycloalkyl, optionally substituted aryl, or optionally substituted heteroaryl; R²³ is hydrogen, —SOₙ₂₃R^{23A}, —SOᵥ₂₃NR^{23B}R^{23C}, —NHNR^{23B}R^{23C}, —ONR^{23B}R^{23C}, —NHC(O)NHNR^{23B}R^{23C}, —NHC(O)NR^{23B}R^{23C}, —NR^{23B}R^{23C}, —C(O)R^{23D}, —C(O)OR^{23D}, —C(O)NR^{23B}R^{23C}, —OR^{23A}, —NR^{23B}SO₂R^{23A}, —NR^{23B}C(O)R^{23D};
—NR^{23B}C(O)OR^{23D}, —NR^{23B}OR^{23D}, optionally substituted alkyl, optionally substituted heteroalkyl, optionally substituted cycloalkyl, optionally substituted heterocycloalkyl, optionally substituted aryl, or optionally substituted heteroaryl; R²⁴ is hydrogen, —SOₙ₂₄R^{24A}, —SOᵥ₂₄NR^{24B}R^{24C}, NHNR^{24B}R^{24C}, ONR^{24B}R^{24C}, —NHC(O)NHNR^{24B}R^{24C}, —NHC(O)NR^{24B}R^{24C}, —NR^{24B}R^{24C}, —C(O)R^{24D}, —C(O)OR^{24D}, —C(O)NR^{24B}R^{24C}, —OR^{24A}, NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, R^{17D}, R^{18A}, R^{18B}, R^{18C}, R^{18D}, R^{22A}, R^{22B}, R^{22C}, R^{22D}, R^{23A}, R^{23B}, R^{23C}, and R^{23D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl;
n17, n18, n22, and n23 are each independently 0, 1, 2, 3, or 4; and
v17, v18, v22, and v23 are each independently 1 or 2.

Embodiment 79. The compound, or a stereoisomer, tautomer, prodrug, or salt thereof, of embodiment 78, wherein the compound comprises the structure of Formula (IV″-b):

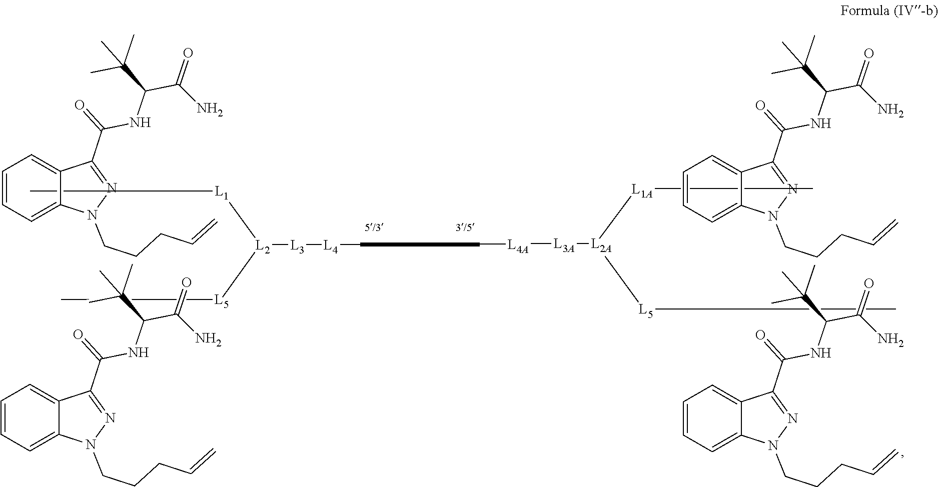

Embodiment 80. The compound, or a stereoisomer, tautomer, prodrug, or salt thereof, of embodiment 78 or 79, wherein the compound comprises the structure of Formula (IV″-b-1):

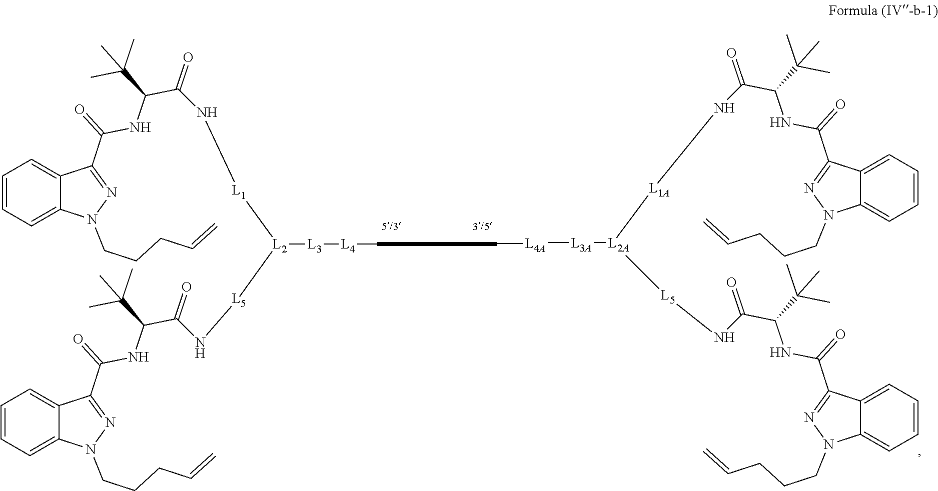

Embodiment 81. A compound, or a stereoisomer, tautomer, prodrug, or salt thereof, comprising the structure of Formula (IV), or a salt thereof:

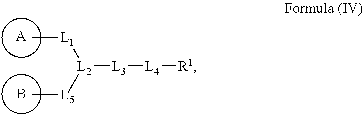

wherein:

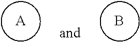

and are each independently a cannabinoid receptor type 1 (CB₁) ligand, or one of

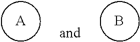

and is a CB₁ ligand, and the other of

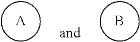

comprises a lipid or a ligand;
each of L₁, L₂, L₃, L₄, and L₅ is independently a linker, a bond, or absent; and
R¹ comprises one or more oligonucleotides, protecting groups, small molecules, proteins, antibodies, and/or peptides.

Embodiment 82. The compound, or a stereoisomer, tautomer, prodrug, or salt thereof, of embodiment 81, wherein

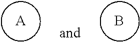

are each independently a CB₁ ligand.

Embodiment 83. The compound, or a stereoisomer, tautomer, prodrug, or salt thereof, of embodiment 81, wherein one or both of the CB₁ ligands is a CB₁ agonist.

Embodiment 84. The compound, or a stereoisomer, tautomer, prodrug, or salt thereof, of embodiment 81, wherein one or both of the CB₁ ligands is a CB₁ antagonist.

Embodiment 85. The compound, or a stereoisomer, tautomer, prodrug, or salt thereof, of embodiment 81, wherein the CB₁ ligand is a selective ligand.

Embodiment 86. The compound, or a stereoisomer, tautomer, prodrug, or salt thereof, of embodiment 81, wherein the CB₁ ligand is a non-selective ligand.

Embodiment 87. The compound, or a stereoisomer, tautomer, prodrug, or salt thereof, of embodiment 81, wherein each of the CB₁ ligands is independently selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

Embodiment 88. The compound, or a stereoisomer, tautomer, prodrug, or salt thereof, of embodiment 81, wherein each of the CB₁ ligands comprises the structure

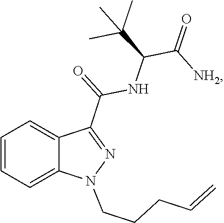 [SMILES: C=CCCCn1nc(C(=O)N[C@H](C(N)=O)C(C)(C)C)c2ccccc21]

or a derivative thereof.

Embodiment 89. The compound, or a stereoisomer, tautomer, prodrug, or salt thereof, of embodiment 81, wherein the compound comprises the structure of Formula (V):

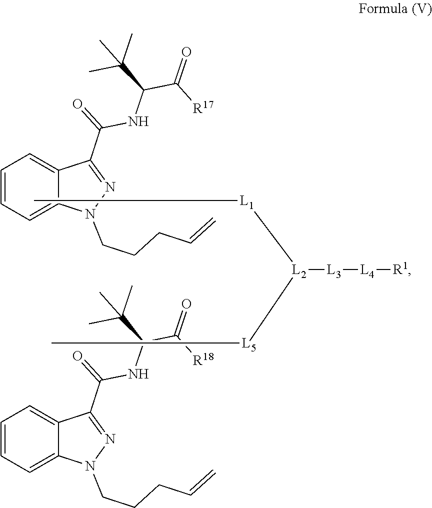

wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R¹⁸, —NHNR^{18B}R^{18C}, —ONR^{18B}R^{18C}, —NHC(O)NHNR^{18B}R^{18C}, —NHC(O)NR^{18B}R^{18C}, —NR^{18B}R^{18C}, —C(O)R^{18D}, —C(O)OR^{18D}, —C(O)NR^{18B}R^{18C}, —OR^{18A}, —NR^{18B}SO₂R^{1A}, —NR^{18B}C(O)R^{18D}, —NR^{18B}C(O)OR^{18D}, —NR^{18B}OR^{18D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, R^{17D}, R^{18A}, R^{18B}, R^{18C}, and R^{18D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl; R^{17B} and R^{17C} or R^{18B} and R^{18C} substituents bonded to the same nitrogen atom may optionally be joined to form a substituted or unsubstituted heterocycloalkyl or substituted or unsubstituted heteroaryl;
n17 and n18 are each independently 0, 1, 2, 3, or 4; and
v17 and v18 are each independently 1 or 2.

Embodiment 90. The compound, or a stereoisomer, tautomer, prodrug, or salt thereof, of embodiment 89, wherein the compound comprises the structure of Formula (V-a):

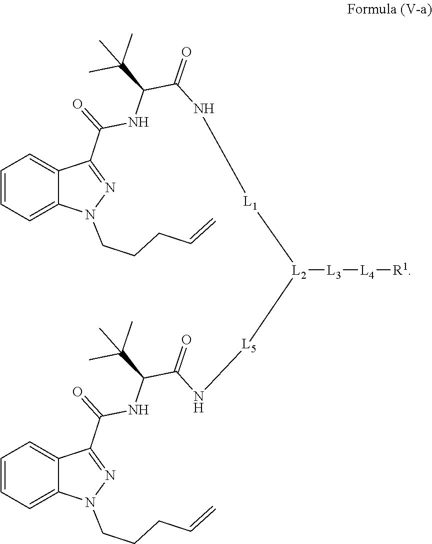

Embodiment 91. The compound, or a stereoisomer, tautomer, prodrug, or salt thereof, of embodiment 81, wherein one of the CB₁ ligands comprises the structure:

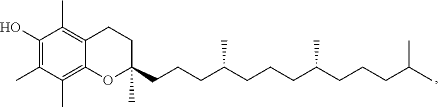 [SMILES: Cc1c(C)c2c(c(C)c1O)CC[C@@](C)(CCC[C@H](C)CCC[C@H](C)CCCC(C)C)O2]

or a derivative thereof, and the other CB₁ ligand comprises the structure:

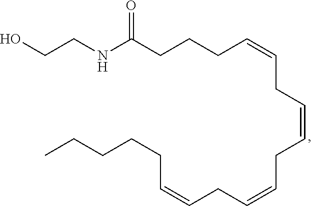 [SMILES: CCCCC/C=C\C/C=C\C/C=C\C/C=C\CCCC(=O)NCCO]

or a derivative thereof.

Embodiment 92. The compound, or a stereoisomer, tautomer, prodrug, or salt thereof, of embodiment 81, wherein the compound comprises the structure of Formula (IX):

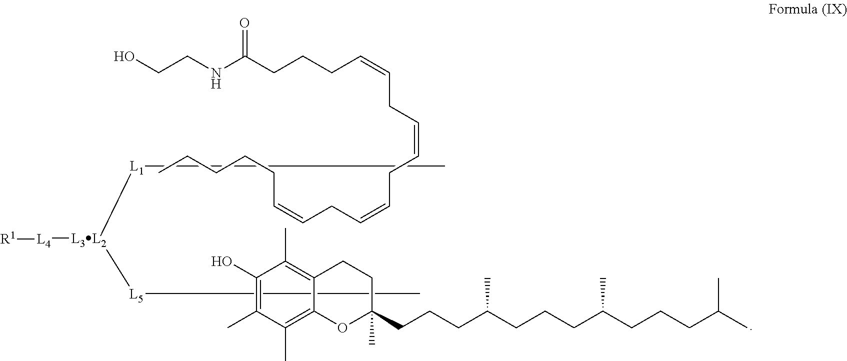

Embodiment 93. The compound, or a stereoisomer, tautomer, prodrug, or salt thereof, of embodiment 92, wherein the compound comprises the structure of Formula (IX-a):

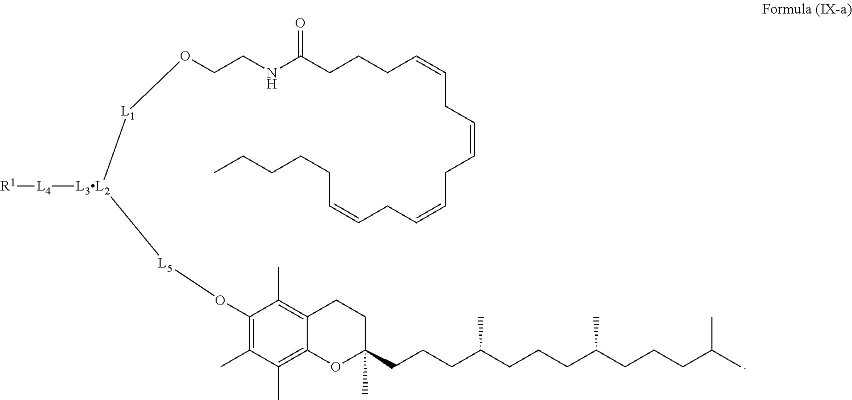

Embodiment 94. The compound, or a stereoisomer, tautomer, prodrug, or salt thereof, of embodiment 81, wherein each of the CB₁ ligands independently comprises the structure:

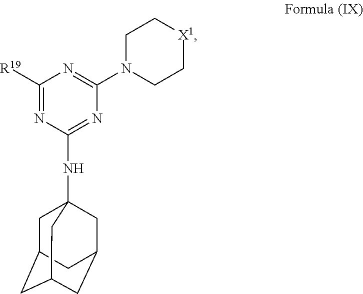

wherein:
X¹ is NR¹⁰ or CR¹¹R¹².
R¹⁰, R¹¹, and R¹² are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D}, —NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, and R^{19D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C} substituents bonded to the same nitrogen atom may optionally be joined to form a substituted or unsubstituted heterocycloalkyl or substituted or unsubstituted heteroaryl;
n19 is 0, 1, 2, 3, or 4; and
v19 is 1 or 2.

Embodiment 95. The compound, or a stereoisomer, tautomer, prodrug, or salt thereof, of embodiment 81, wherein the compound comprises the structure of Formula (XI):

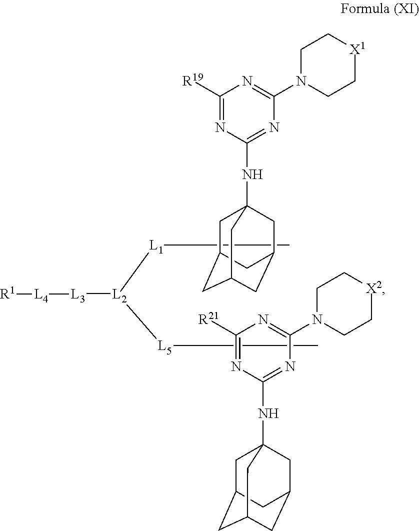

wherein:
X¹ is NR¹⁰ or CR¹¹R¹²;
X² is NR¹³ or CR¹⁴R¹⁵;
R¹⁰, R¹¹, R¹², R¹³, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D}, R^{21A}, R^{21B}, R^{21C}, and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{21B} and R^{21C} substituents bonded to the same nitrogen atom may optionally be joined to form a substituted or unsubstituted heterocycloalkyl or substituted or unsubstituted heteroaryl;
n19 and n21 are each independently 0, 1, 2, 3, or 4; and
v19 and v21 are each independently 1 or 2.

Embodiment 96. The compound, or a stereoisomer, tautomer, prodrug, or salt thereof, of embodiment 95, wherein the compound comprises the structure of Formula (XI-a):

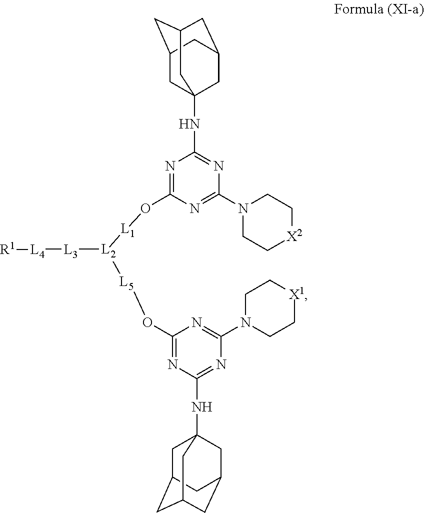

Embodiment 97. The compound, or a stereoisomer, tautomer, prodrug, or salt thereof, of embodiment 95 or 96, wherein:
X¹ is NR⁰;
X² is NR¹³; and
R¹⁰ and R¹³ are each independently hydrogen or optionally substituted alkyl.

Embodiment 98. The compound, or a stereoisomer, tautomer, prodrug, or salt thereof, of embodiment 97, wherein:
R¹⁰ and R¹³ are each independently hydrogen, —CH₃, or —CH₂CH₂F.

Embodiment 99. The compound, or a stereoisomer, tautomer, prodrug, or salt thereof, of embodiment 95 or 96, wherein:
X¹ is CR¹¹R¹²;
X² is CR¹⁴R¹⁵; and
R¹¹, R¹², R¹⁴, and R¹⁵ are each independently hydrogen or optionally substituted alkyl.

Embodiment 100. The compound, or a stereoisomer, tautomer, prodrug, or salt thereof, of embodiment 99, wherein:
R¹² and R¹⁵ are each independently hydrogen, —CH₃, or —CH₂CH₂F; and
R¹¹ and R¹⁴ are each independently hydrogen.

Embodiment 101. The compound, or a stereoisomer, tautomer, prodrug, or salt thereof, of any one of embodiments 81-100, wherein each of L₁, L₂, L₃, L₄, and L₅ is independently absent, a bond, an optionally substituted alkyl linker, an optionally substituted polyethylene glycol (PEG) linker, an optionally substituted heteroalkyl linker, an optionally substituted heteroaryl linker, oxygen, optionally substituted nitrogen, an amide, a phosphodiester bond, or a phosphorothioate bond.

Embodiment 102. The compound, or a stereoisomer, tautomer, prodrug, or salt thereof, of embodiment 101, wherein L₁ and L₅ are each an optionally substituted PEG linker.

Embodiment 103. The compound, or a stereoisomer, tautomer, prodrug, or salt thereof, of embodiment 102, wherein L₁ and L₅ are each an optionally substituted PEG linker of 1, 2, 3, 4, 5, 6, 7, 8, 9, or 10 PEG units in length.

Embodiment 104. The compound, or a stereoisomer, tautomer, prodrug, or salt thereof, of any one of embodiments 81-103, wherein L₂ is an optionally substituted heteroalkyl linker.

Embodiment 105. The compound, or a stereoisomer, tautomer, prodrug, or salt thereof, of embodiment 104, wherein the heteroalkyl linker is substituted with one or more ═O substituents.

Embodiment 106. The compound, or a stereoisomer, tautomer, prodrug, or salt thereof, of any one of embodiments 81-105, wherein L₂ comprises the structure

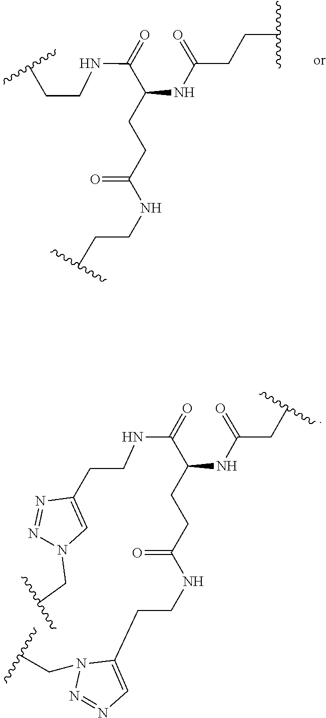 [SMILES: *CCNC(=O)CC[C@H](NC(=O)CC*)C(=O)NCC* ; *CC(=O)N[C@@H](CCC(=O)NCCc1cnnn1C*)C(=O)NCCc1cn(C*)nn1]

Embodiment 107. The compound, or a stereoisomer, tautomer, prodrug, or salt thereof, of any one of embodiments 81-106, wherein L₃ is an optionally substituted heteroaryl linker.

Embodiment 108. The compound, or a stereoisomer, tautomer, prodrug, or salt thereof, of any one of embodiments 81-106, wherein L₃ is an optionally substituted partially unsaturated heteroaryl linker or optionally substituted partially unsaturated heterocycloalkyl linker.

Embodiment 109. The compound, or a stereoisomer, tautomer, prodrug, or salt thereof, of embodiment 108, wherein L₃ comprises the structure

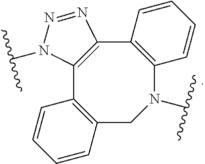 [SMILES: *N1Cc2ccccc2-c2c(nnn2*)-c2ccccc21]

Embodiment 110. The compound, or a stereoisomer, tautomer, prodrug, or salt thereof, of any one of embodiments 81-109, wherein L₄ is an optionally substituted heteroalkyl linker.

Embodiment 111. The compound, or a stereoisomer, tautomer, prodrug, or salt thereof, of embodiment 110, wherein the heteroalkyl linker is substituted with one or more ═O substituents.

Embodiment 112. The compound, or a stereoisomer, tautomer, prodrug, or salt thereof, of embodiment 110 or 111, wherein L₄ comprises the structure

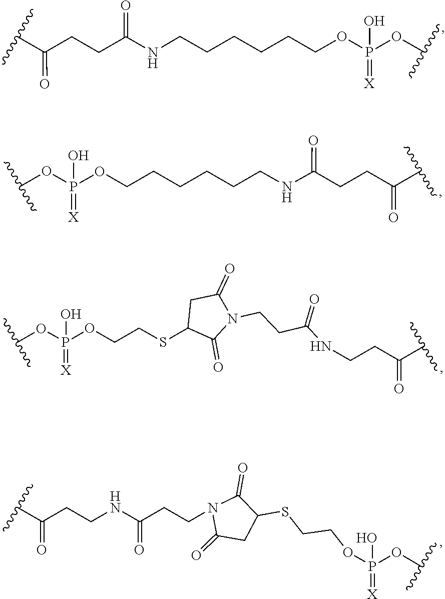

wherein X is O or S.

Embodiment 113. The compound, or a stereoisomer, tautomer, prodrug, or salt thereof, of embodiment 112, wherein L₄ comprises the structure

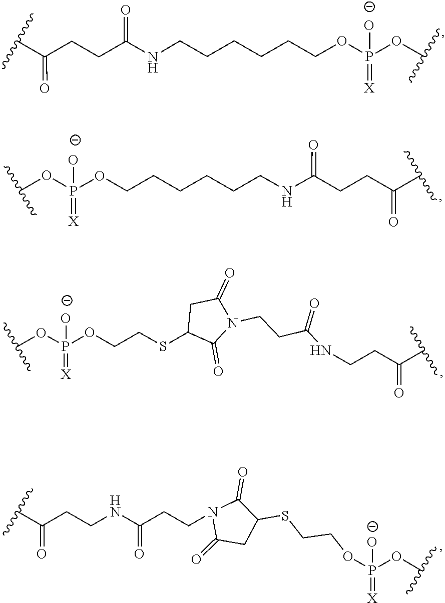

wherein X is O or S.

Embodiment 114. The compound, or a stereoisomer, tautomer, prodrug, or salt thereof, of embodiment 112 or 113, wherein X is S.

Embodiment 115. The compound, or a stereoisomer, tautomer, prodrug, or salt thereof, of embodiment 112 or 113, wherein X is O.

Embodiment 116. The compound of any one or embodiments 81-115, wherein L₁, L₂, L₃, L₄, and L₅ together comprise the structure

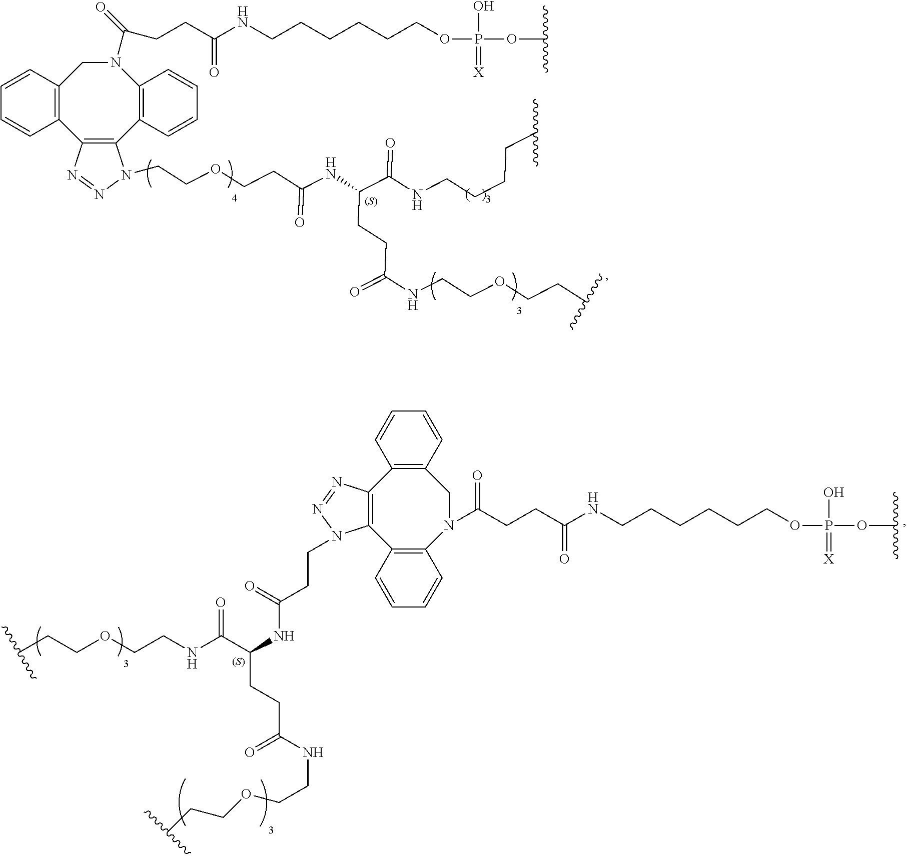

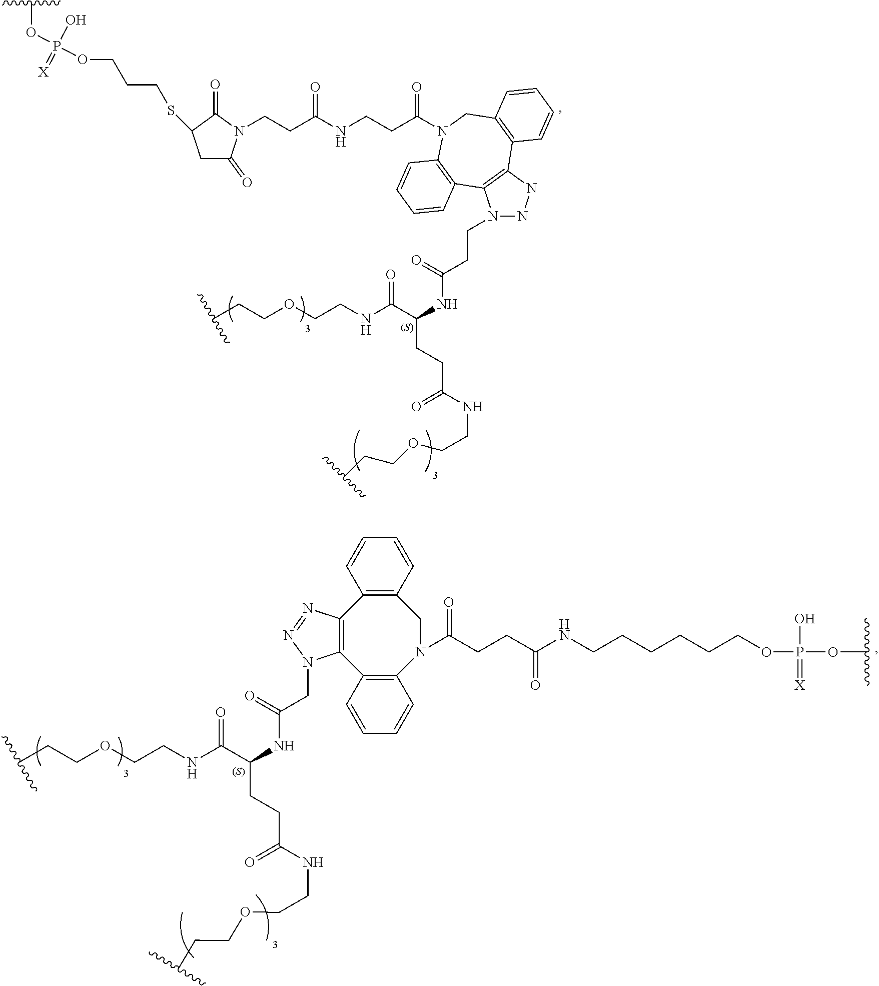

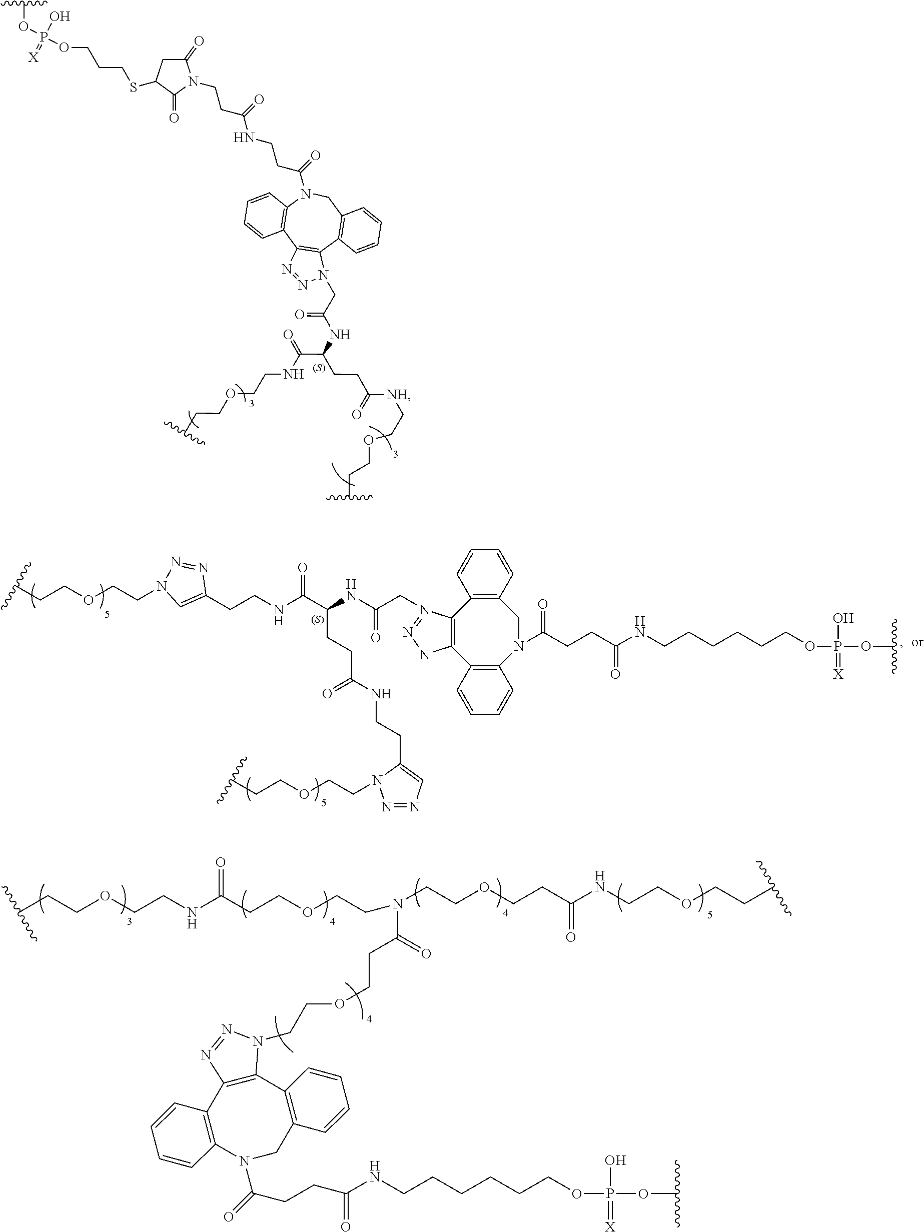

wherein X is O or S.

Embodiment 117. The compound of embodiment 116, wherein L₁, L₂, L₃, L₄, and L₅ together comprise the structure

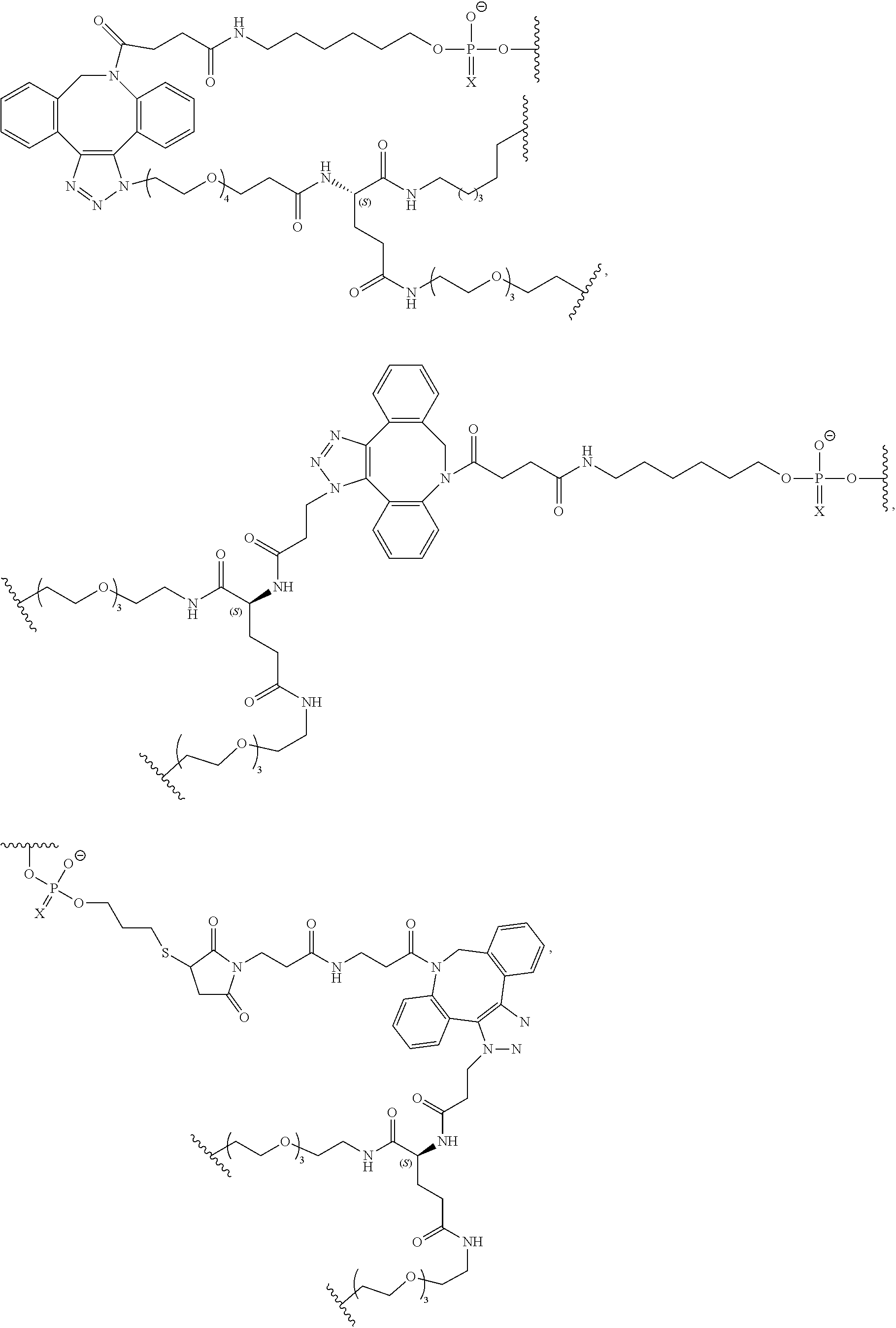

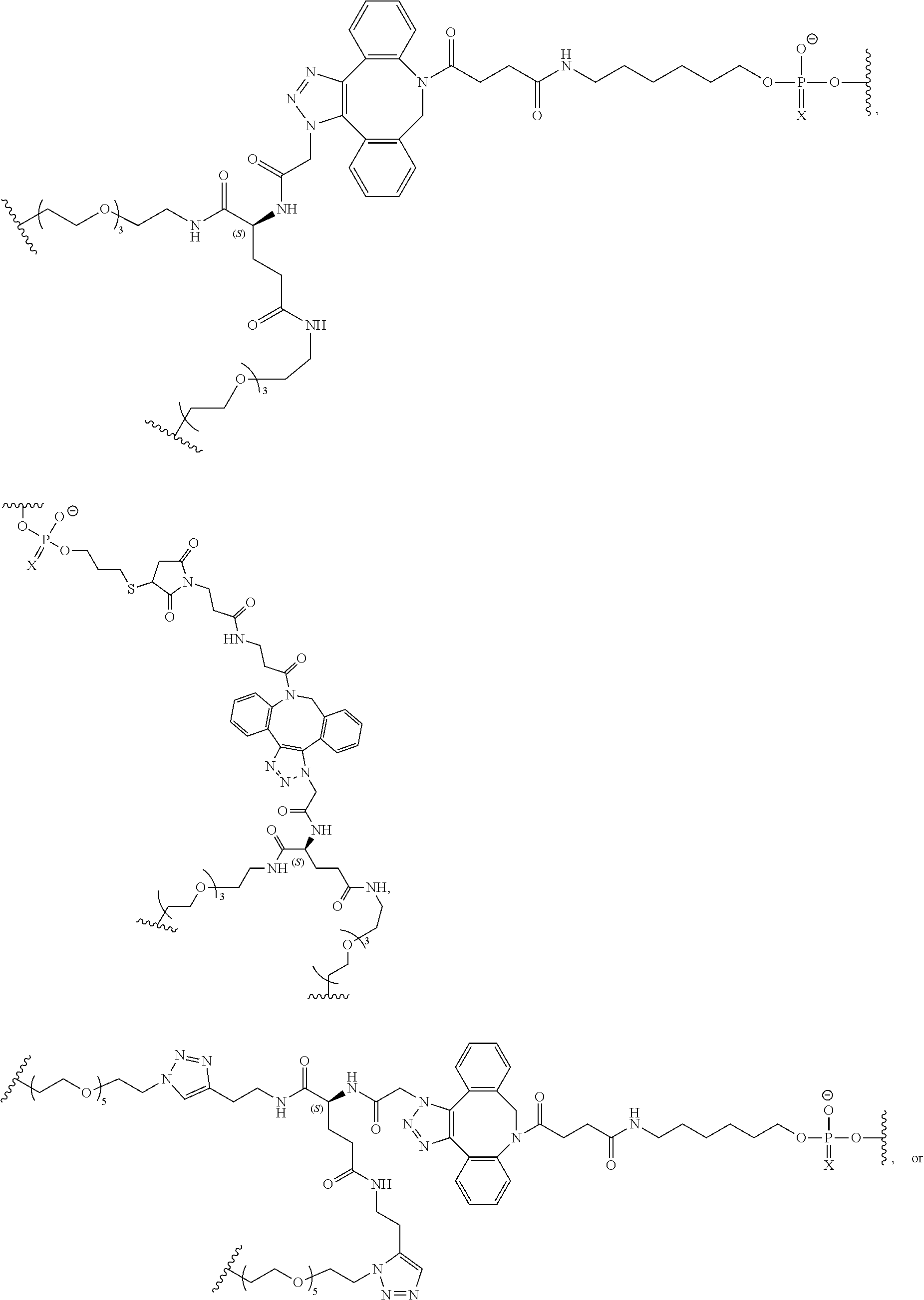

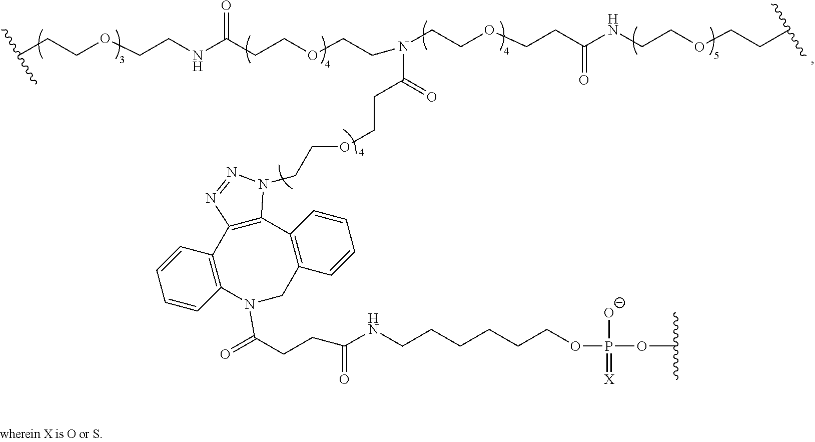

Embodiment 118. A compound, or a stereoisomer, tautomer, prodrug, or salt thereof, comprising the structure of Formula (VI), or a salt thereof:

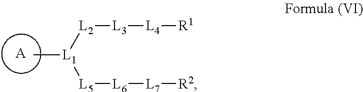

wherein:

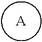

is a cannabinoid receptor type 1 (CB₁) ligand;
each of L₁, L₂, L₃, L₄, L₅, L₆, and L₇ is independently a linker, a bond, or absent; and
R¹ and R² each independently comprise one or more oligonucleotides, protecting groups, small molecules, proteins, antibodies, and/or peptides.

Embodiment 119. The compound, or a stereoisomer, tautomer, prodrug, or salt thereof, of embodiment 118, wherein the CB₁ ligand is a CB₁ agonist.

Embodiment 120. The compound, or a stereoisomer, tautomer, prodrug, or salt thereof, of embodiment 118, wherein the CB₁ ligand is a CB₁ antagonist.

Embodiment 121. The compound, or a stereoisomer, tautomer, prodrug, or salt thereof, of embodiment 118, wherein the CB₁ ligand is selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

Embodiment 122. The compound, or a stereoisomer, tautomer, prodrug, or salt thereof, of embodiment 118, wherein the CB₁ ligand comprises the structure

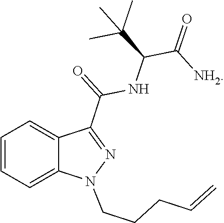 [SMILES: C=CCCCn1nc(C(=O)N[C@H](C(N)=O)C(C)(C)C)c2ccccc21]

Embodiment 123. The compound, or a stereoisomer, tautomer, prodrug, or salt thereof, of embodiment 118, wherein the compound comprises the structure of Formula (VII):

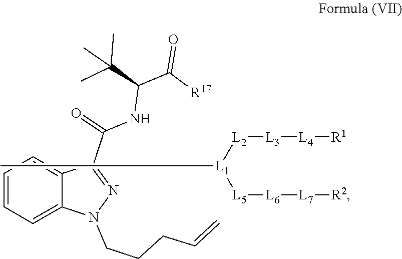

wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{7D}, —C(O)OR^{17D}, —C(O)NR^{17B}R^{17C}, —OR^{7A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, and R^{17D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl
n17 is 0, 1, 2, 3, or 4; and
v17 is 1 or 2.

Embodiment 124. The compound, or a stereoisomer, tautomer, prodrug, or salt thereof, of embodiment 123, wherein the compound comprises the structure of Formula (VII-a):

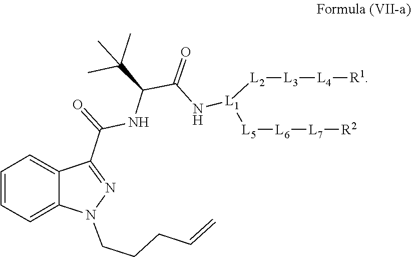

Embodiment 125. The compound, or a stereoisomer, tautomer, prodrug, or salt thereof, of any one of embodiments 118-124, wherein each of L₁, L₂, L₃, L₄, L₅, L₆, and L₇ is independently absent, a bond, an optionally substituted alkyl linker, an optionally substituted polyethylene glycol (PEG) linker, an optionally substituted heteroalkyl linker, an optionally substituted heteroaryl linker, an optionally substituted saturated or partially unsaturated heterocycloalkyl linker, oxygen, optionally substituted nitrogen, an amide, a phosphodiester bond, or a phosphorothioate bond.

Embodiment 126. The compound, or a stereoisomer, tautomer, prodrug, or salt thereof, of embodiment 125, wherein L₁ is an optionally substituted PEG linker.

Embodiment 127. The compound, or a stereoisomer, tautomer, prodrug, or salt thereof, of embodiment 126, wherein L₁ is an optionally substituted PEG linker which is 1, 2, 3, 4, 5, 6, 7, 8, 9, or 10 PEG units in length.

Embodiment 128. The compound, or a stereoisomer, tautomer, prodrug, or salt thereof, of any one of embodiments 125-127, wherein L₂ and L₅ are each independently an optionally substituted PEG linker.

Embodiment 129. The compound, or a stereoisomer, tautomer, prodrug, or salt thereof, of embodiment 128, wherein the L₂ and L₅ are each independently an optionally substituted PEG linker three or four PEG units in length.

Embodiment 130. The compound, or a stereoisomer, tautomer, prodrug, or salt thereof, of any one of embodiments 125-129, wherein L₁, L₂, and L₅ together comprise the structure

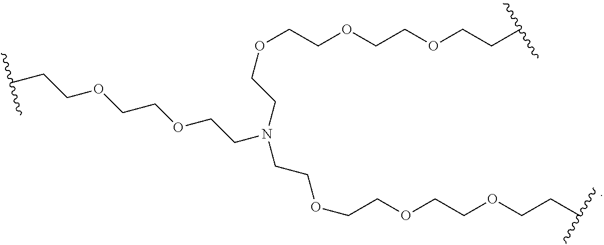 [SMILES: *CCOCCOCCOCCN(CCOCCOCC*)CCOCCOCCOCC*]

Embodiment 131. The compound, or a stereoisomer, tautomer, prodrug, or salt thereof, of any one of embodiments 125-129, wherein L₃ and L₆ are each independently an optionally substituted heteroaryl linker or an optionally substituted heterocycloalkyl linker.

Embodiment 132. The compound, or a stereoisomer, tautomer, prodrug, or salt thereof, of embodiment 131, wherein L₃ and L₆ are each independently an optionally substituted partially unsaturated heteroaryl linker or an optionally substituted partially unsaturated heterocycloalkyl linker.

Embodiment 133. The compound, or a stereoisomer, tautomer, prodrug, or salt thereof, of embodiment 131 or 132, wherein L₃ and L₆ each comprise the structure

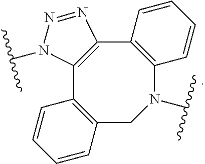 [SMILES: *N1Cc2ccccc2-c2c(nnn2*)-c2ccccc21]

Embodiment 134. The compound, or a stereoisomer, tautomer, prodrug, or salt thereof, of any one of embodiments 125-133, wherein L₄ and L₇ are each independently an optionally substituted heteroalkyl linker.

Embodiment 135. The compound, or a stereoisomer, tautomer, prodrug, or salt thereof, of embodiment 134, wherein the heteroalkyl linker is substituted with one or more ═O substituents.

Embodiment 136. The compound, or a stereoisomer, tautomer, prodrug, or salt thereof, of embodiment 134 or 135, wherein L₄ and L₇ each comprise the structure

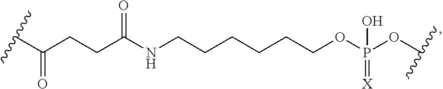

wherein X is O or S.

Embodiment 137. The compound, or a stereoisomer, tautomer, prodrug, or salt thereof, of embodiment 136, wherein L₄ and L₇ each comprise the structure

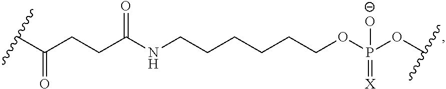

wherein X is O or S.

Embodiment 138. The compound, or a salt thereof, of any one of embodiments 1-137, wherein R¹ comprises an oligonucleotide.

Embodiment 139. The compound, or a stereoisomer, tautomer, prodrug, or salt thereof, of embodiment 138, wherein the oligonucleotide is attached at its 5′ end.

Embodiment 140. The compound, or a stereoisomer, tautomer, prodrug, or salt thereof, of embodiment 138, wherein the oligonucleotide is attached at its 3′ end.

Embodiment 141. The compound, or a stereoisomer, tautomer, prodrug, or salt thereof, of embodiment 138, wherein the oligonucleotide is attached at an internal position on the oligonucleotide.

Embodiment 142. The compound, or a stereoisomer, tautomer, prodrug, or salt thereof, of embodiment 141, wherein the internal position is an internucleoside linkage.

Embodiment 143. The compound, or a stereoisomer, tautomer, prodrug, or salt thereof, of any one of embodiments 1-142, wherein R¹ comprises an oligonucleotide conjugated to one or more additional CB₁ ligands.

Embodiment 144. The compound, or a stereoisomer, tautomer, prodrug, or salt thereof, of embodiment 143, wherein the oligonucleotide is conjugated to two, three, four, five, or more than five additional CB₁ ligands.

Embodiment 145. The compound, or a stereoisomer, tautomer, prodrug, or salt thereof, of embodiment 143 or 144, wherein the additional CB₁ ligands are conjugated to the oligonucleotide at the 5′ end of the oligonucleotide, the 3′ end of the oligonucleotide, one or more internal positions on the oligonucleotide, or any combination thereof.

Embodiment 146. The compound of any one of embodiments 1-145, or a stereoisomer, tautomer, prodrug, or salt thereof, wherein the oligonucleotide is a modified oligonucleotide.

Embodiment 147. The compound, or a salt thereof, of any one of embodiments 1-146, wherein L₁, L₂, L₃, and L₄ together comprise the structure:

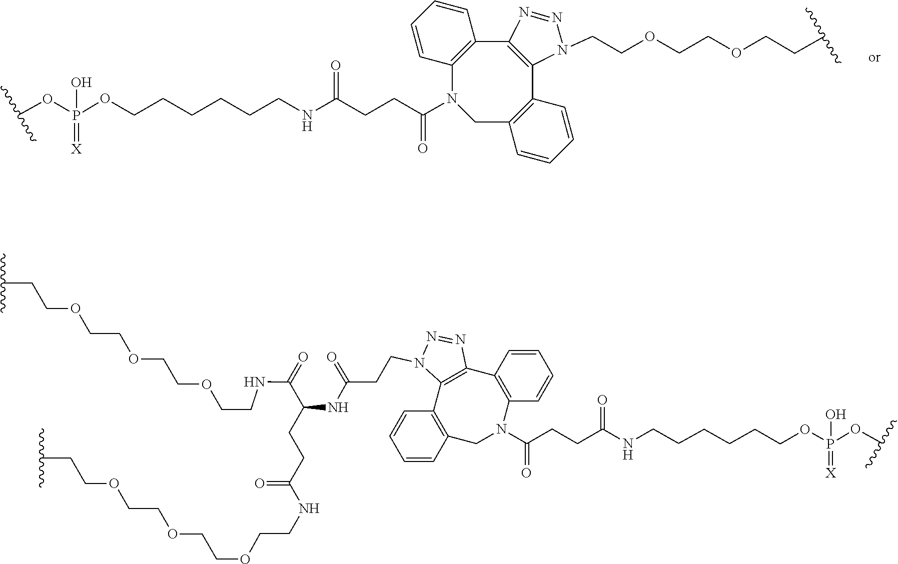

wherein X is or S.

Embodiment 148. The compound, or a stereoisomer, tautomer, prodrug, or salt thereof, of embodiment 147, wherein L₁, L₂, L₃, and L₄ together comprise the structure:

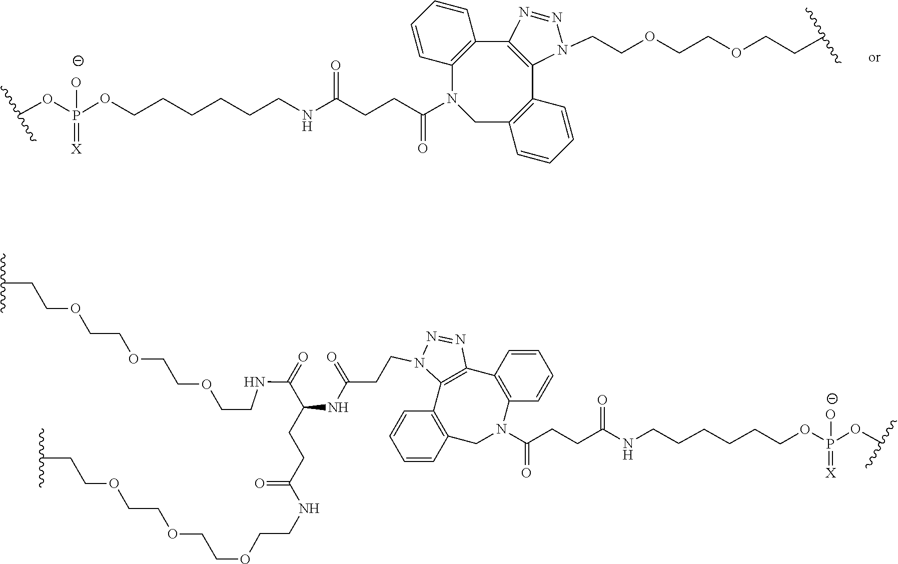

wherein X is O or S.

Embodiment 149. The compound, or a salt thereof, of any one of embodiments 118-146, wherein L₁, L₂, L₃, L₄, L₅, L₆, and L₇ together comprise the structure:

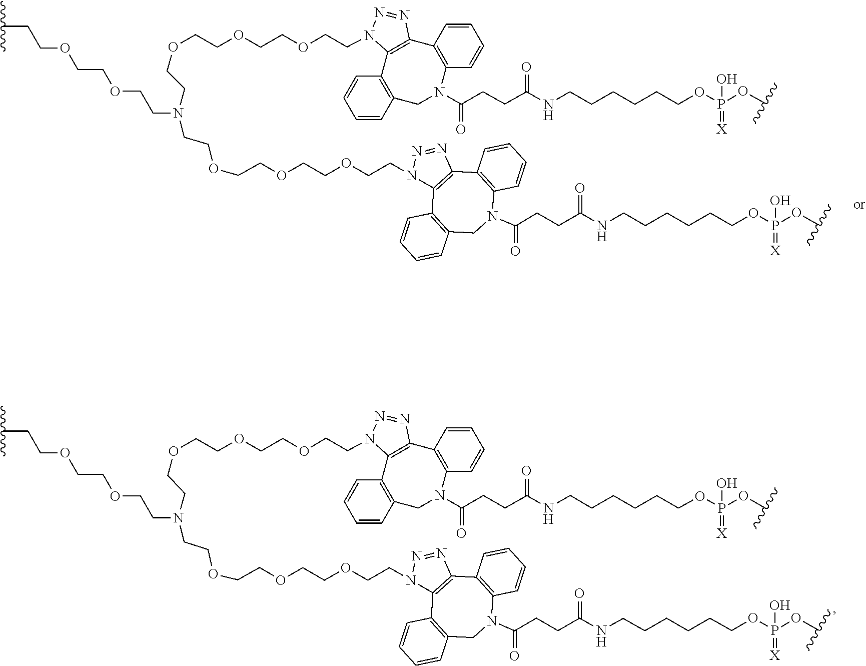

wherein X is O or S.

Embodiment 150. The compound, or a stereoisomer, tautomer, prodrug, or salt thereof, of embodiment 149, wherein L₁, L₂, L₃, L₄, L₅, L₆, and L₇ together comprise the structure:

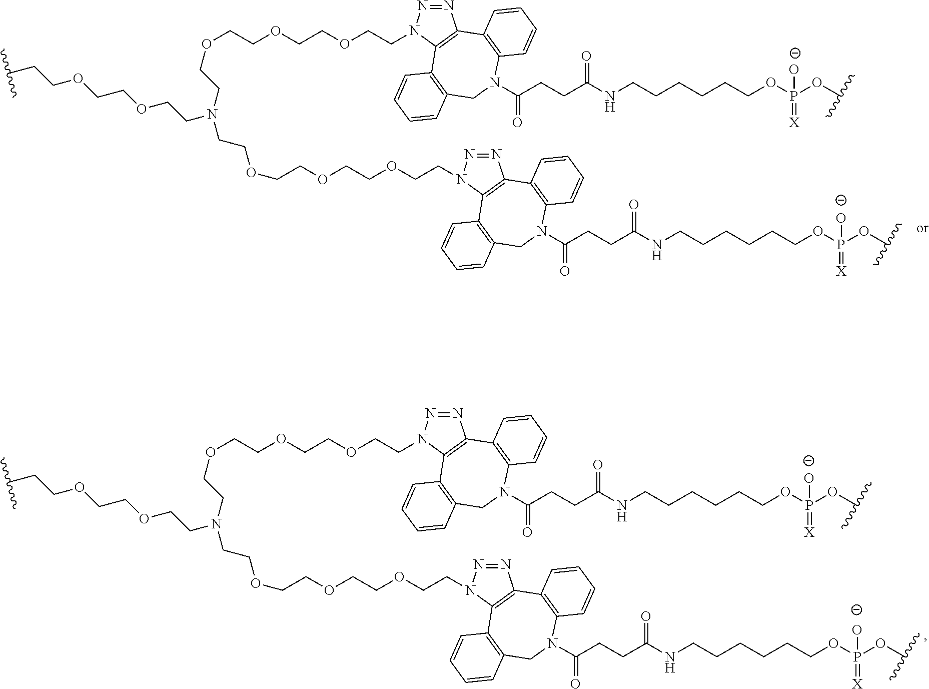

wherein X is O or S.

Embodiment 151. A compound, or a stereoisomer, tautomer, prodrug, or salt thereof, comprising the structure:

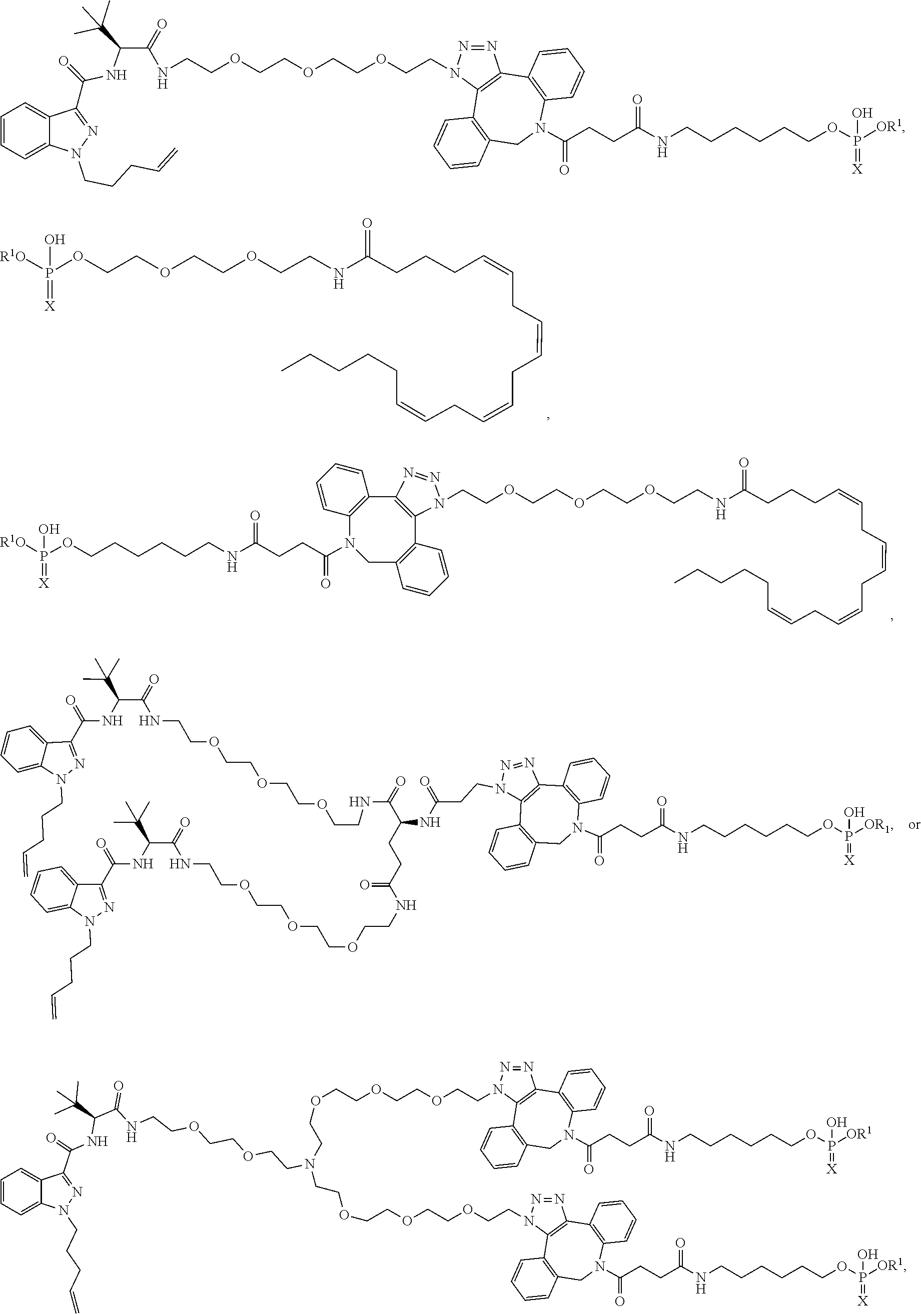

wherein:
R¹ and R² each independently comprise one or more oligonucleotides, protecting groups, small molecules, proteins, antibodies, and/or peptides; and
X is O or S.

Embodiment 152. The compound of embodiment 151, wherein the compound comprises the structure:

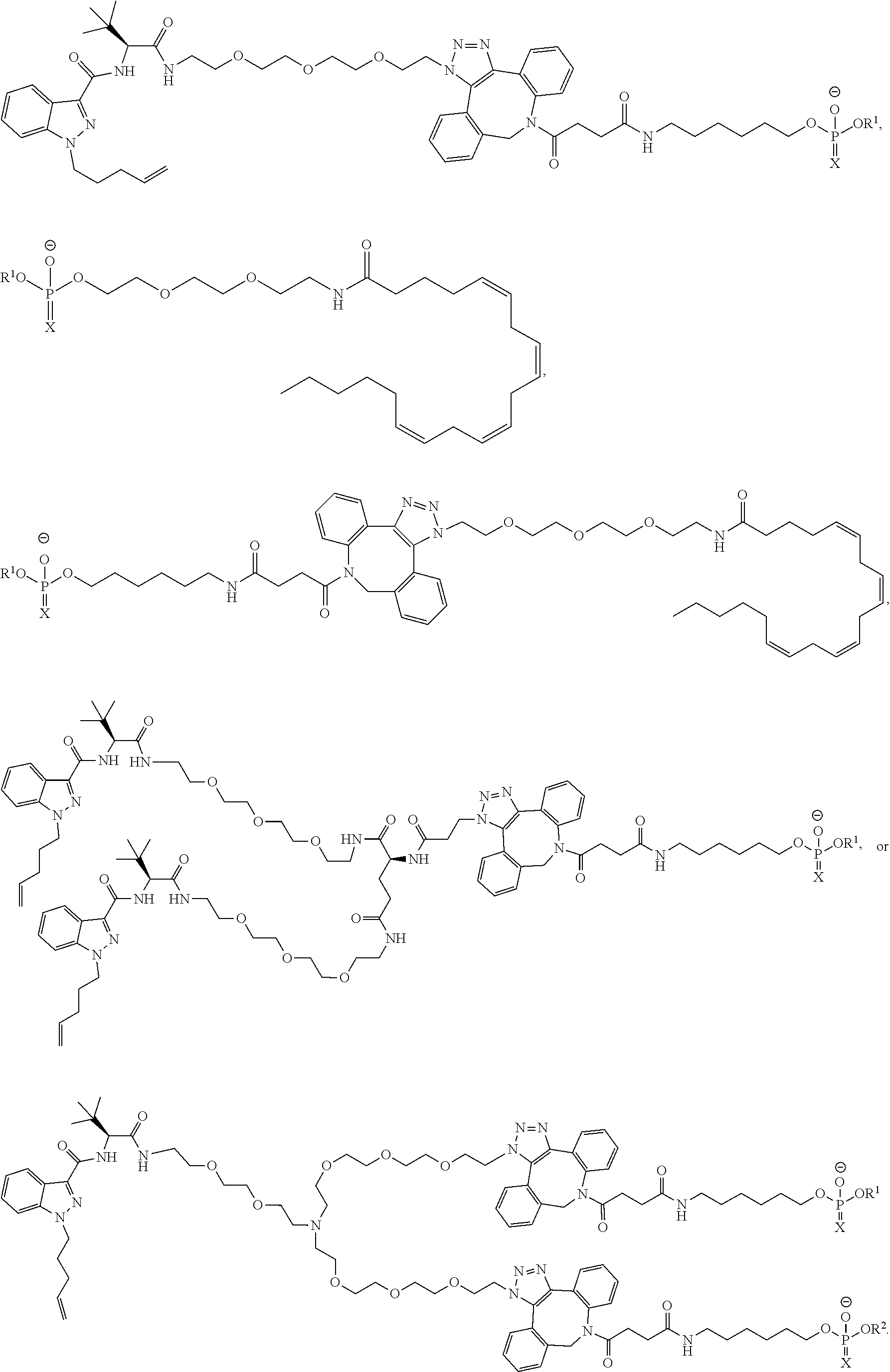

Embodiment 153. A compound, or a stereoisomer, tautomer, prodrug, or salt thereof, comprising the structure:

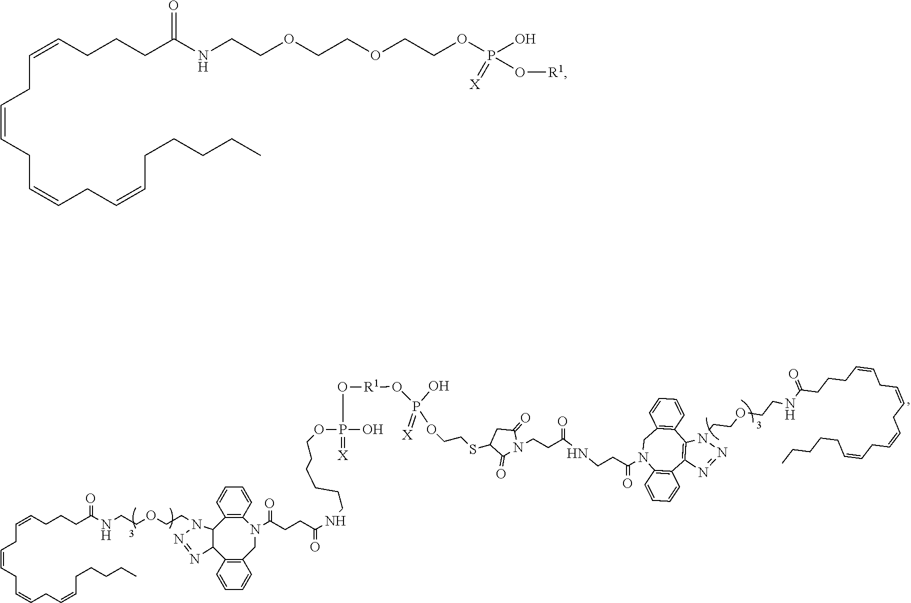

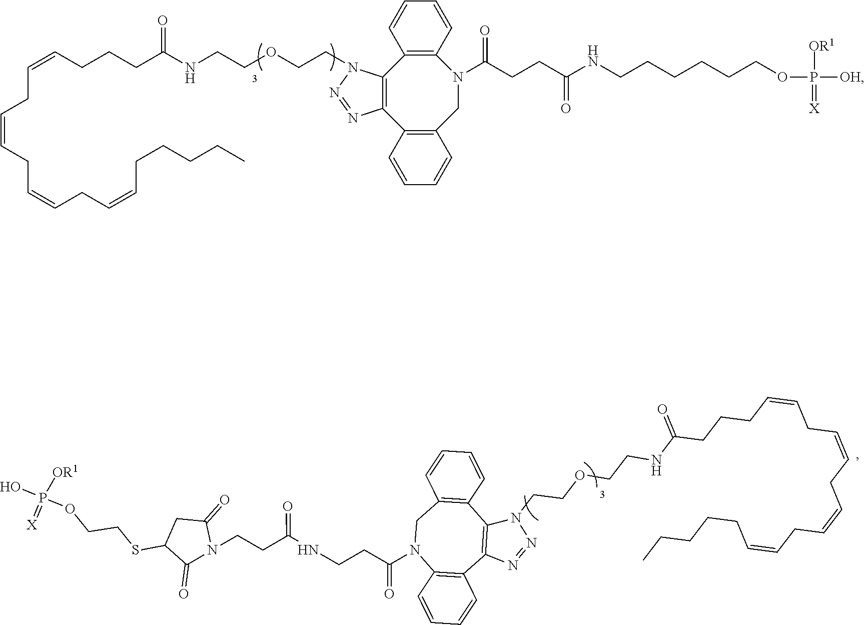

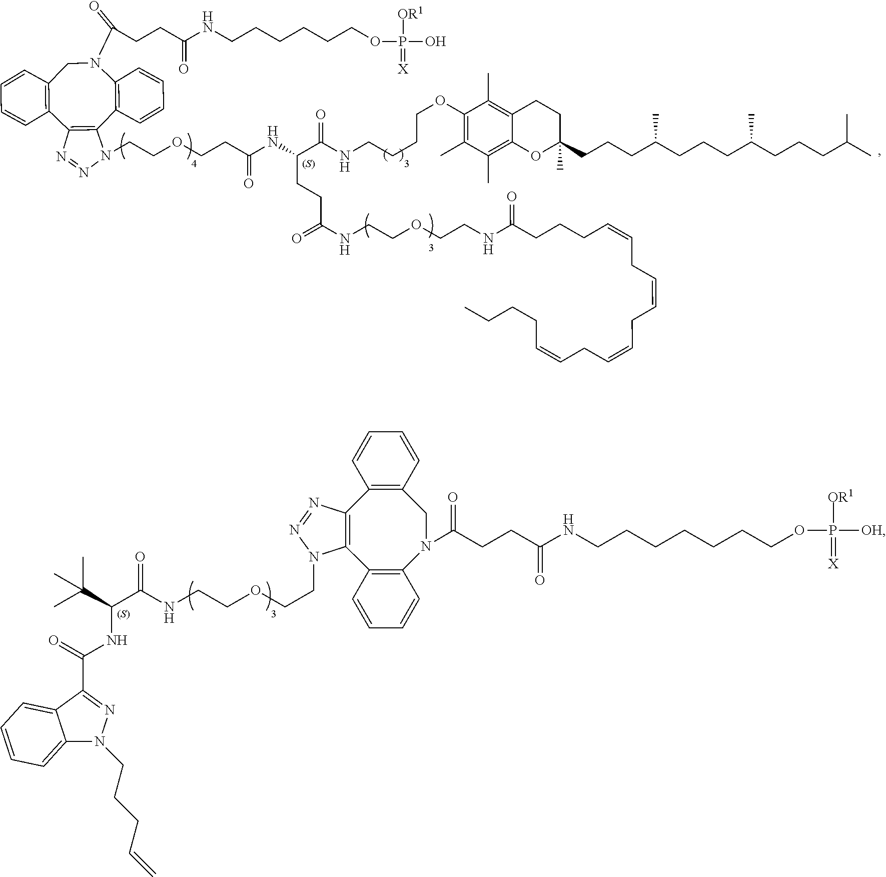

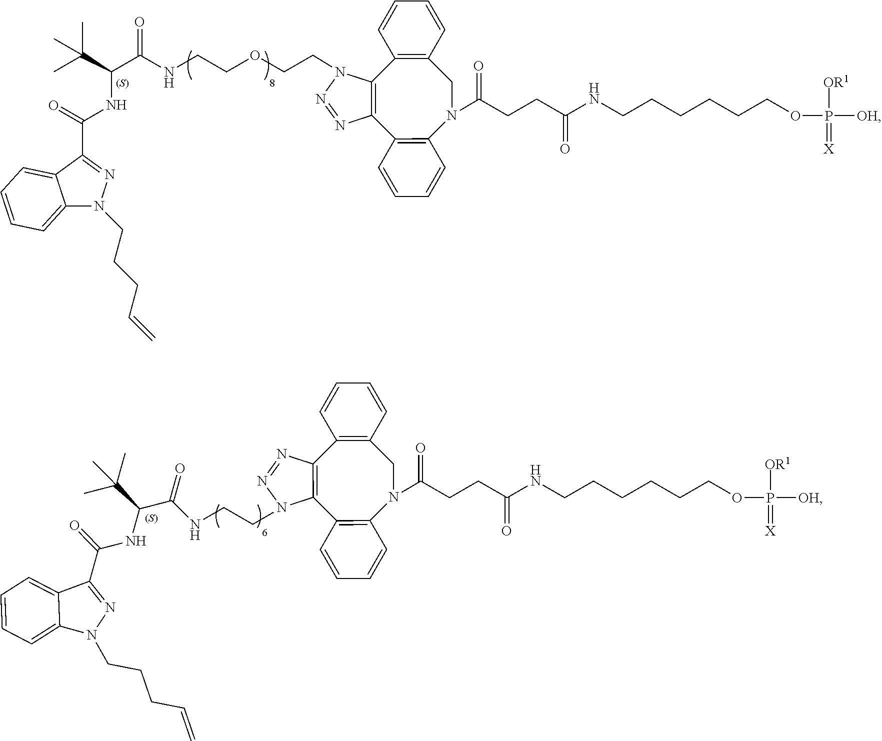

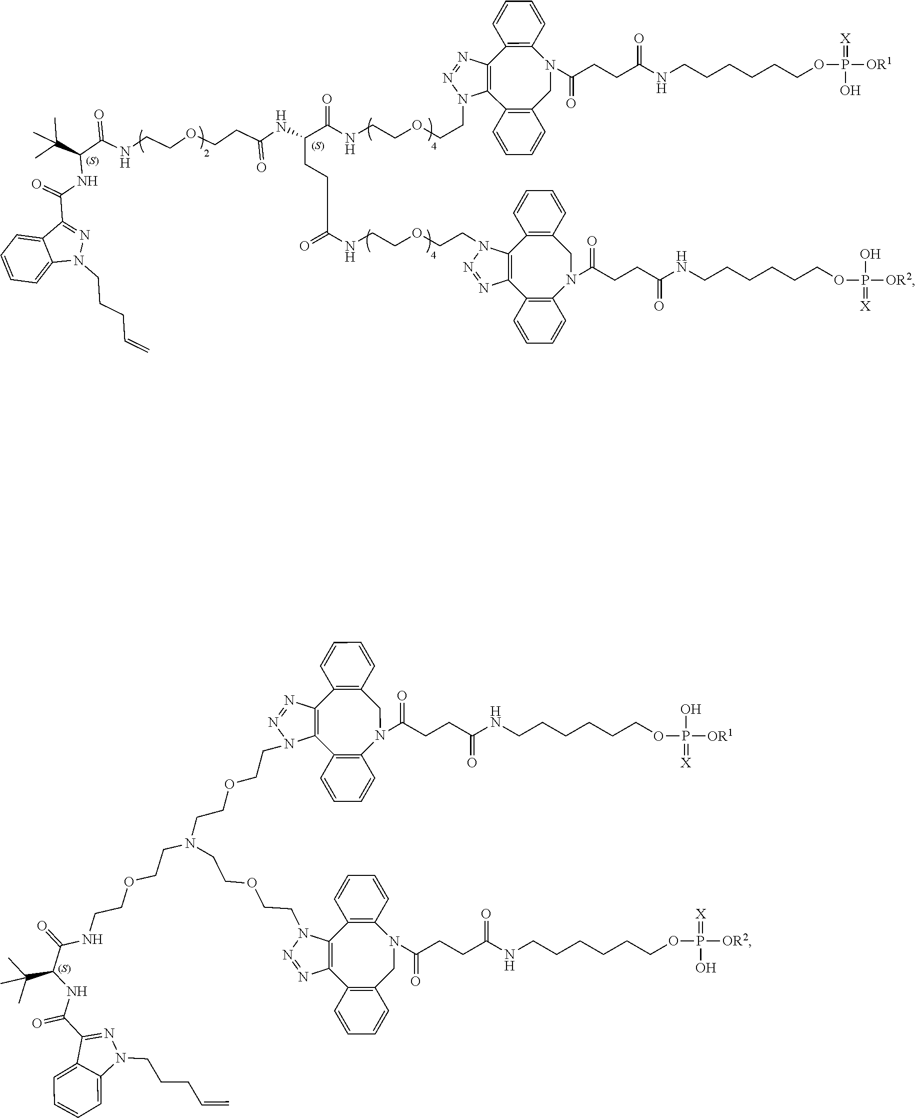

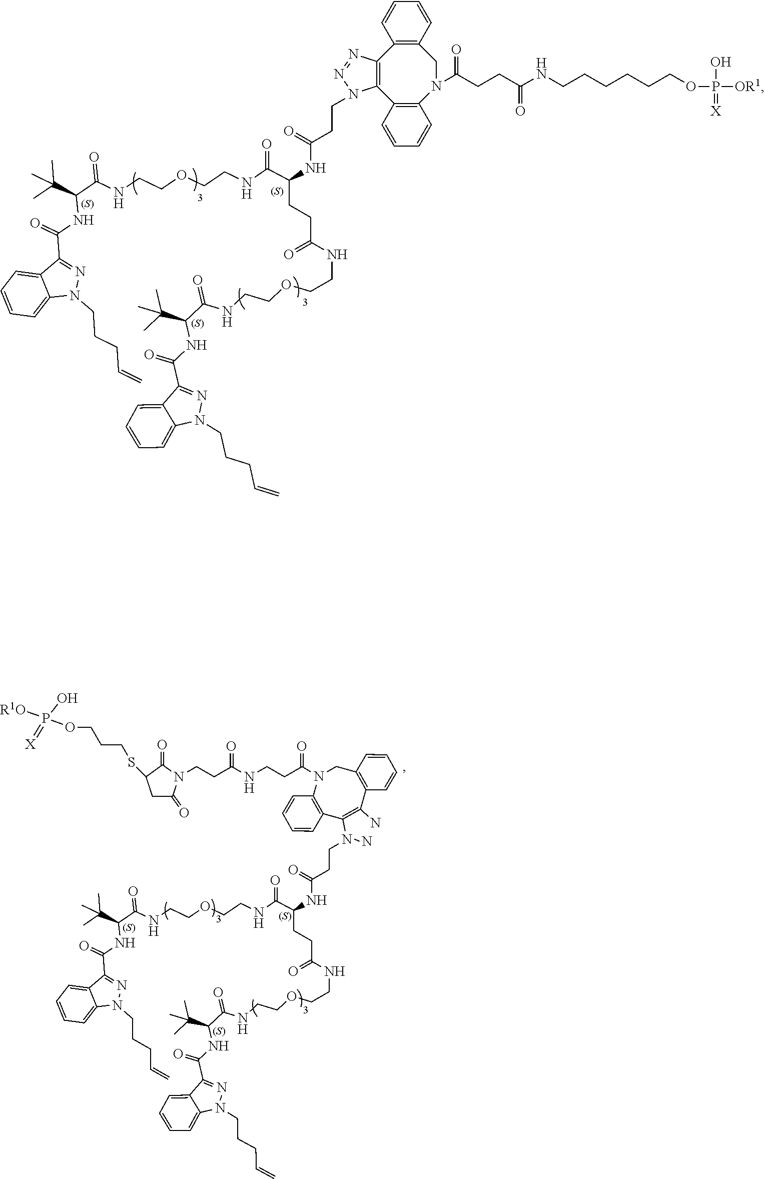

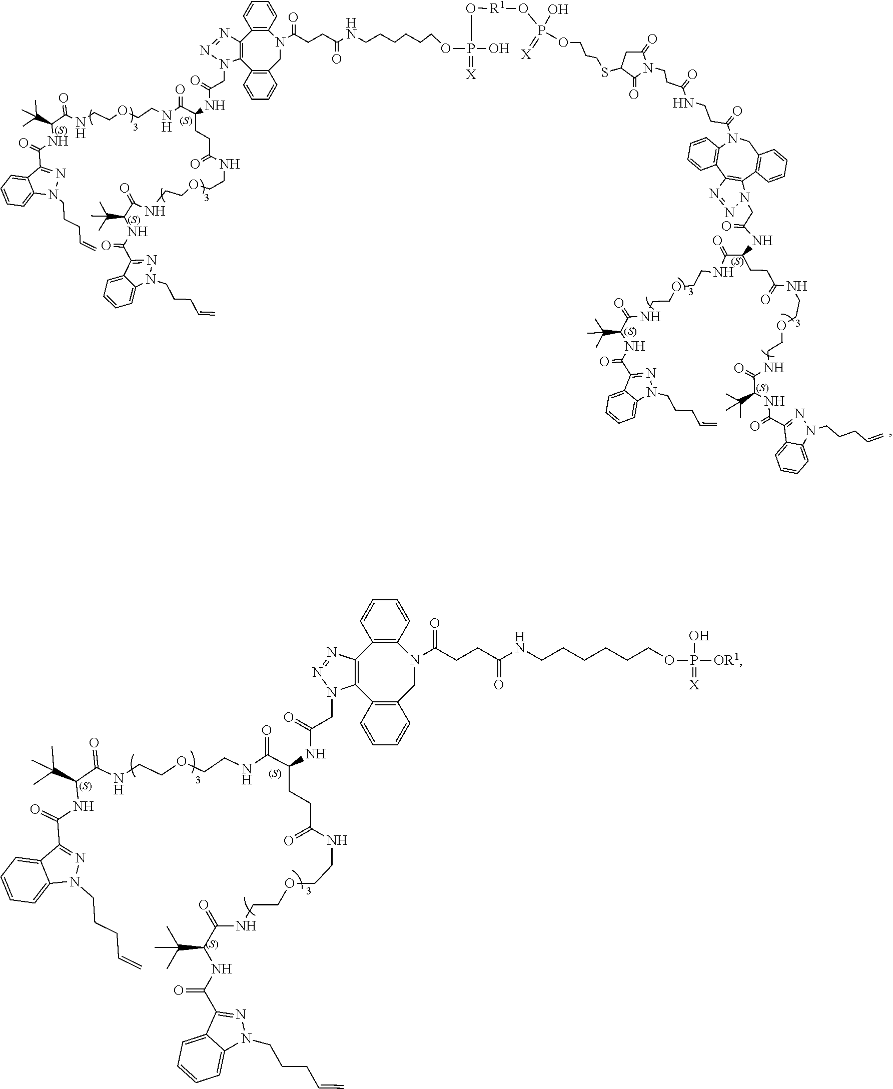

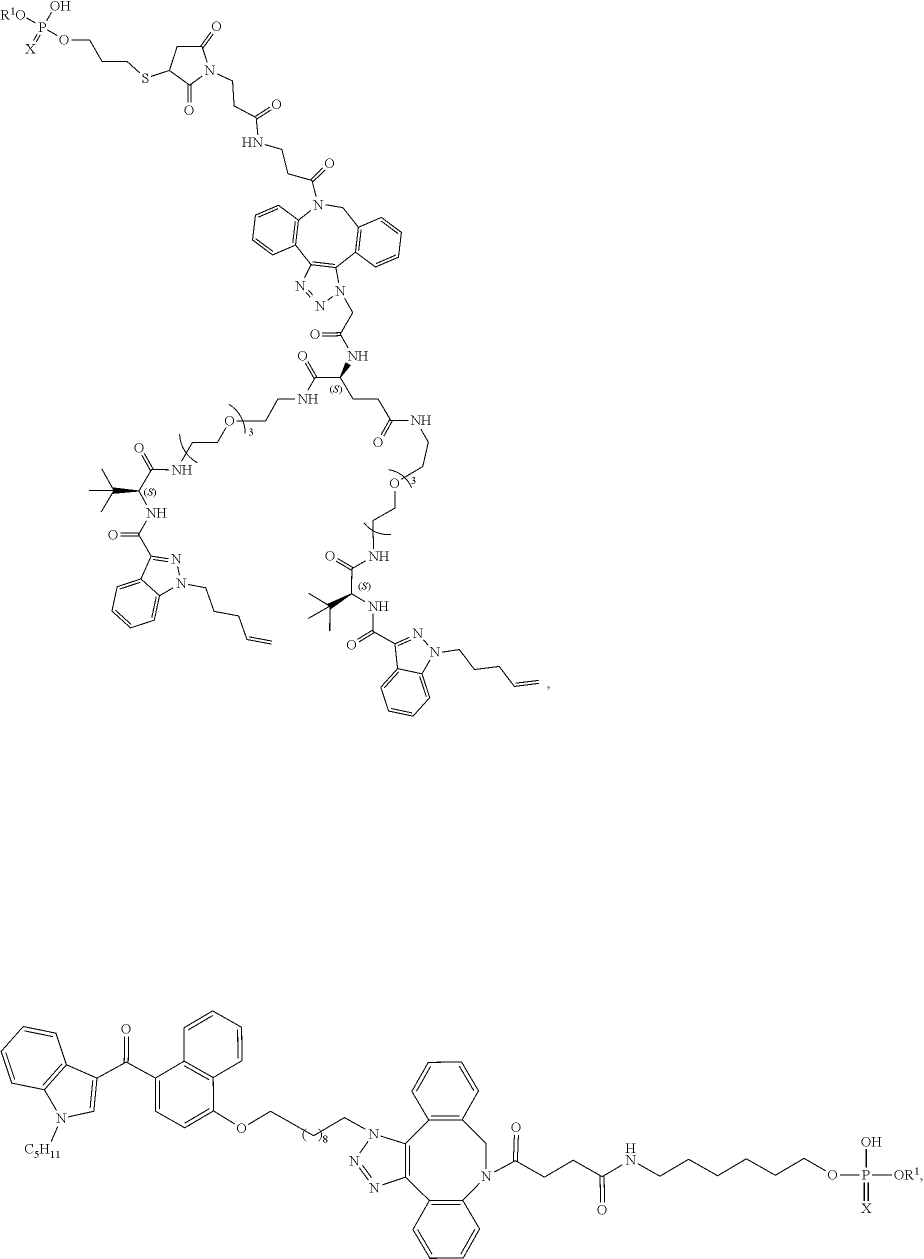

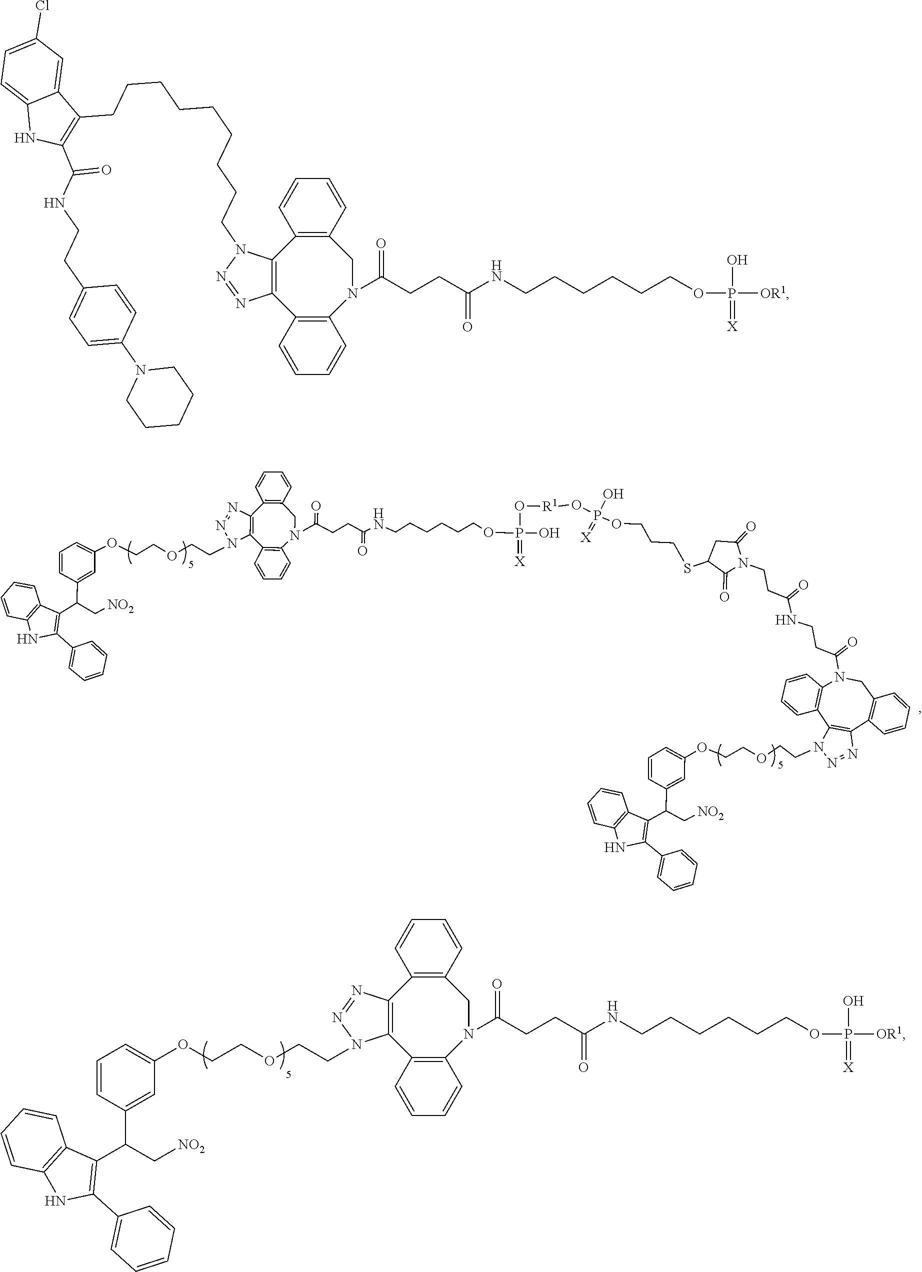

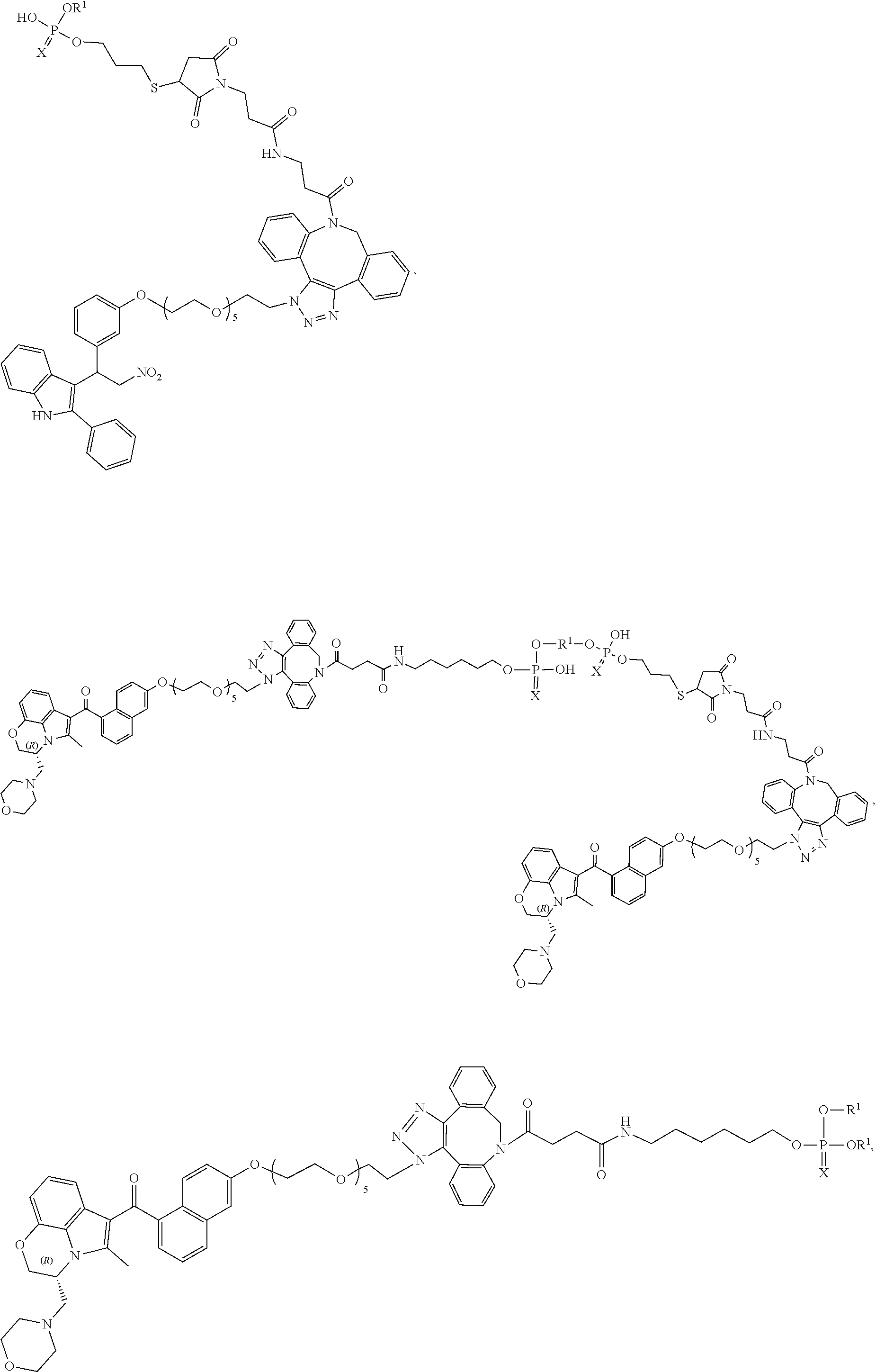

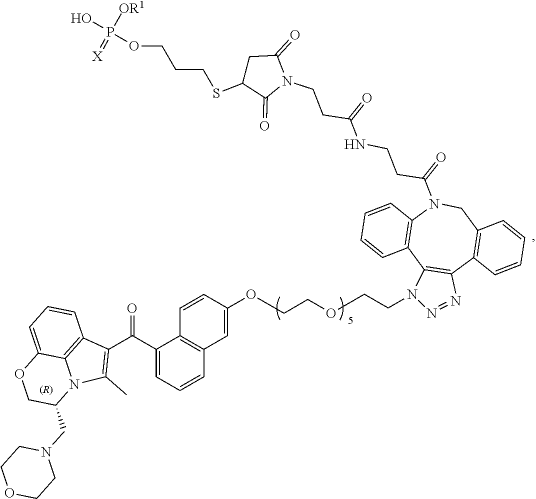

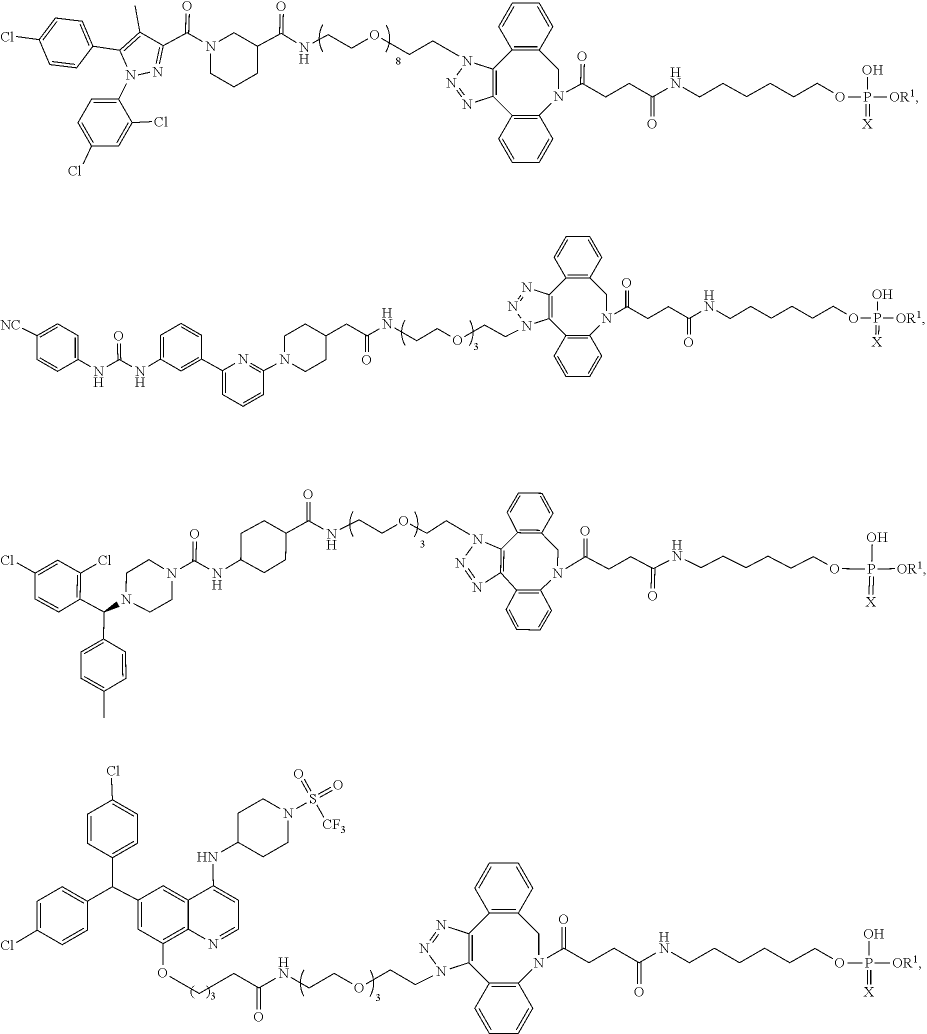

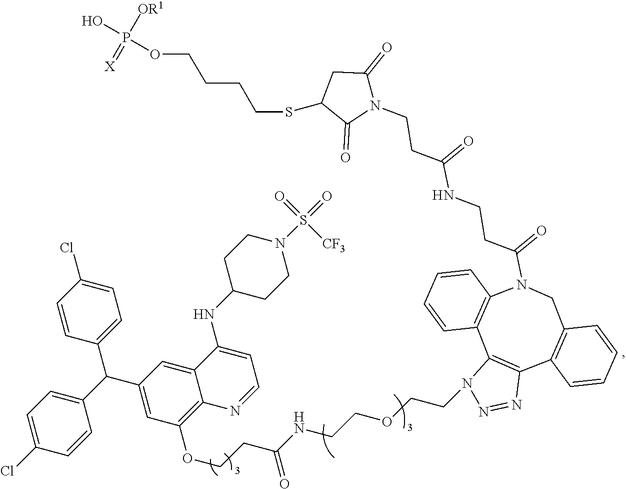

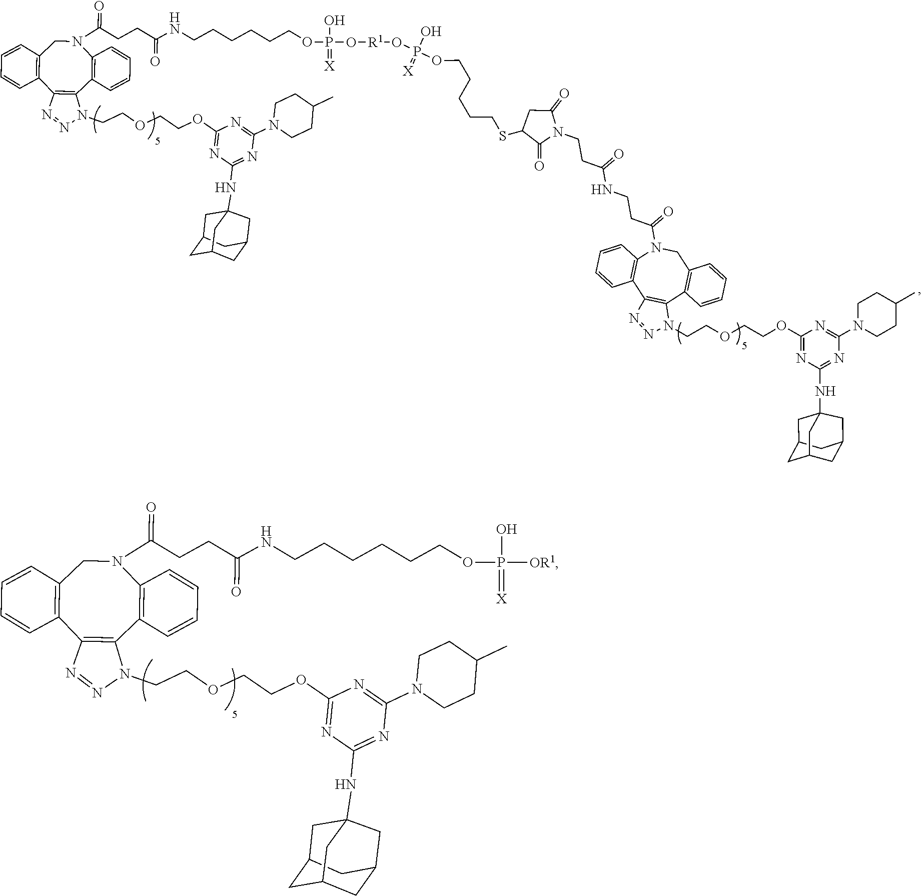

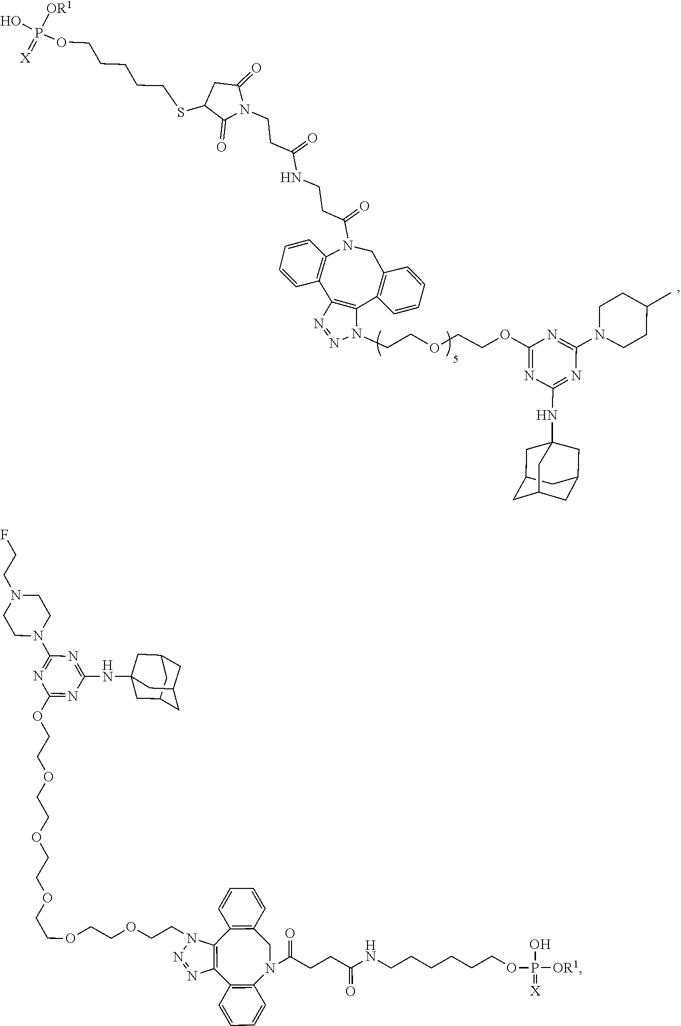

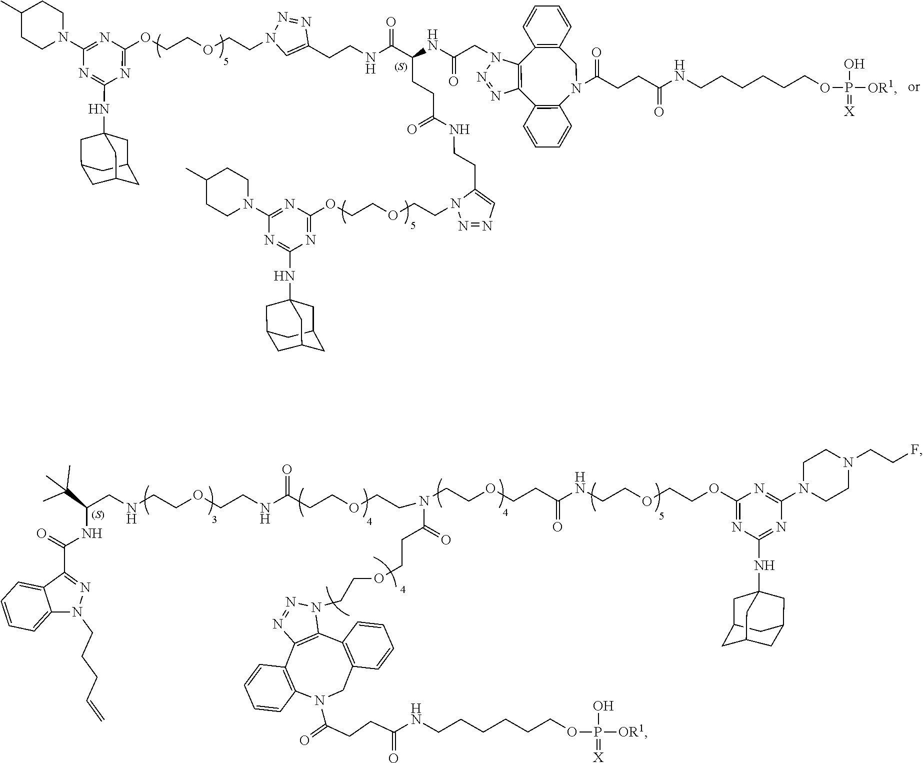

wherein:
R¹ and R² each independently comprise one or more oligonucleotides, protecting groups, small molecules, proteins, antibodies, and/or peptides; and
X is O or S.

Embodiment 154. The compound, or a stereoisomer, tautomer, prodrug, or salt thereof, of any one of embodiments 1-149, wherein the compound comprises the structure:

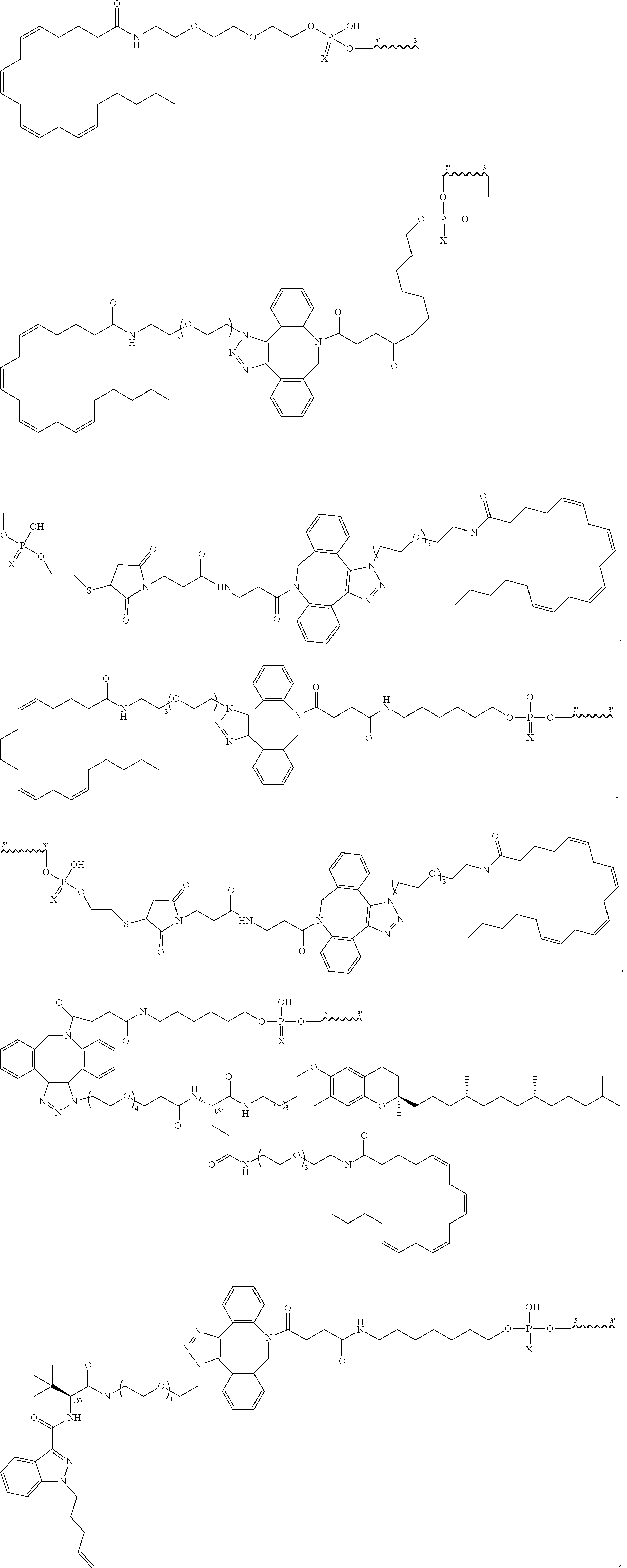

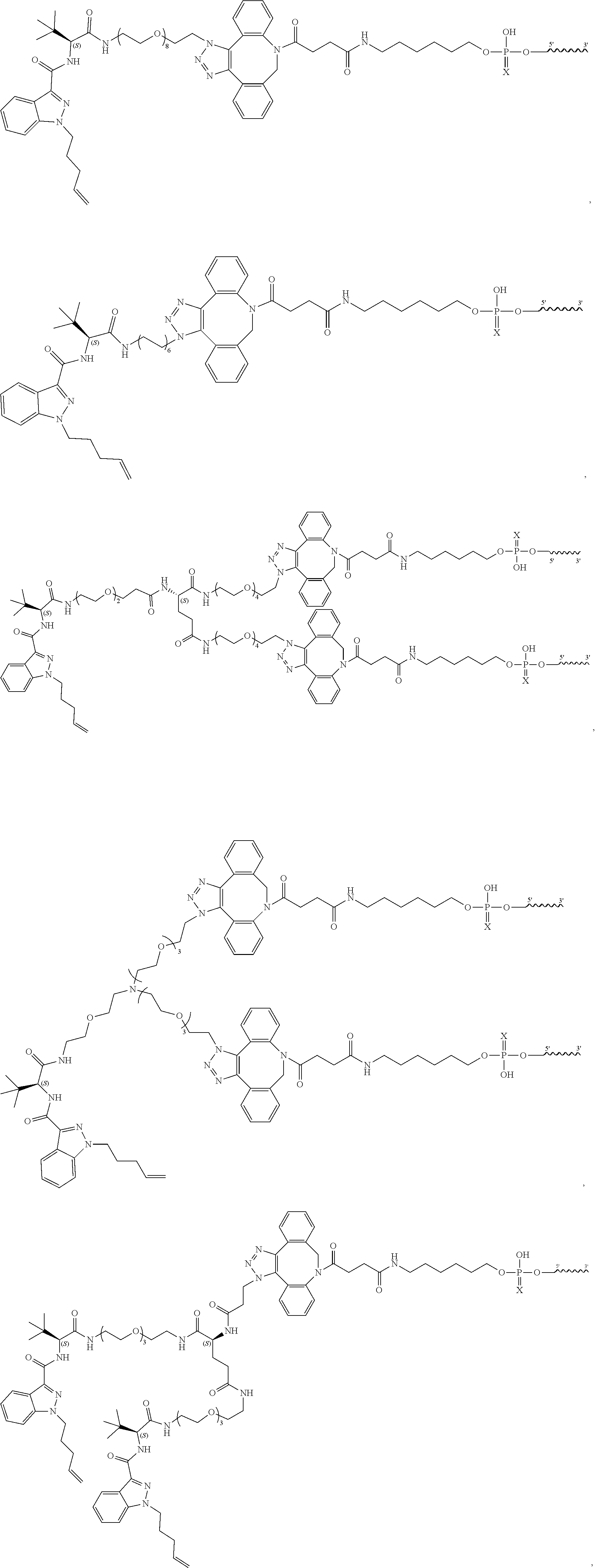

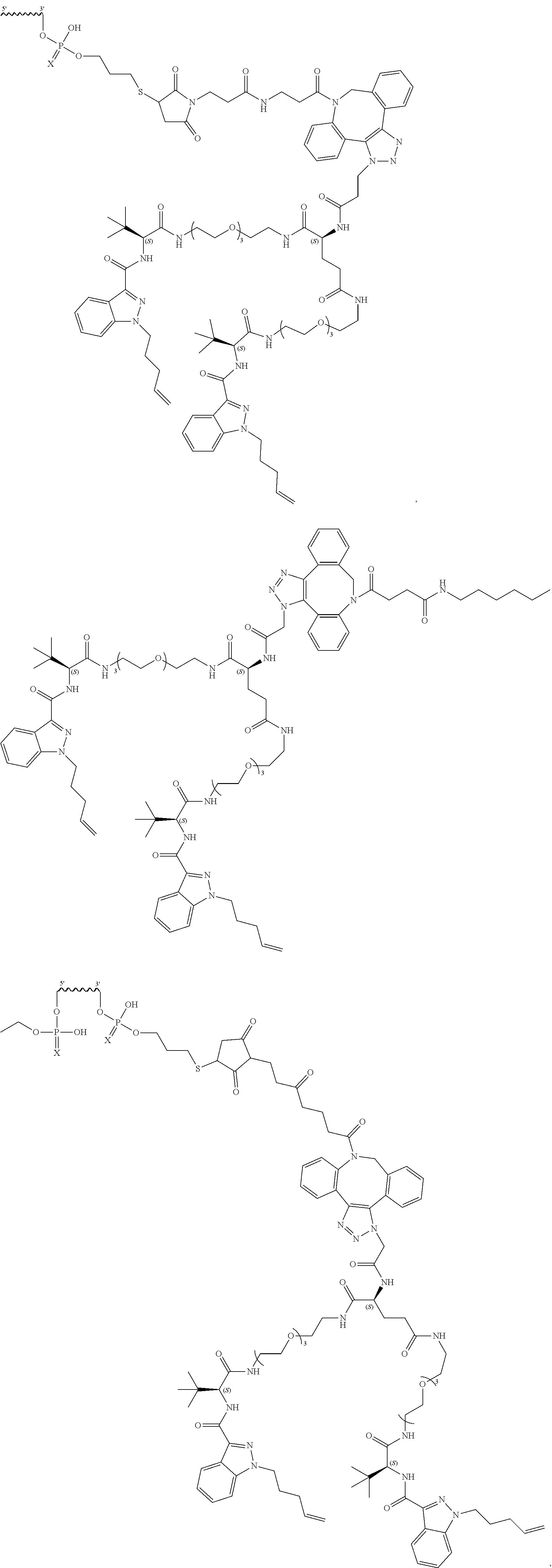

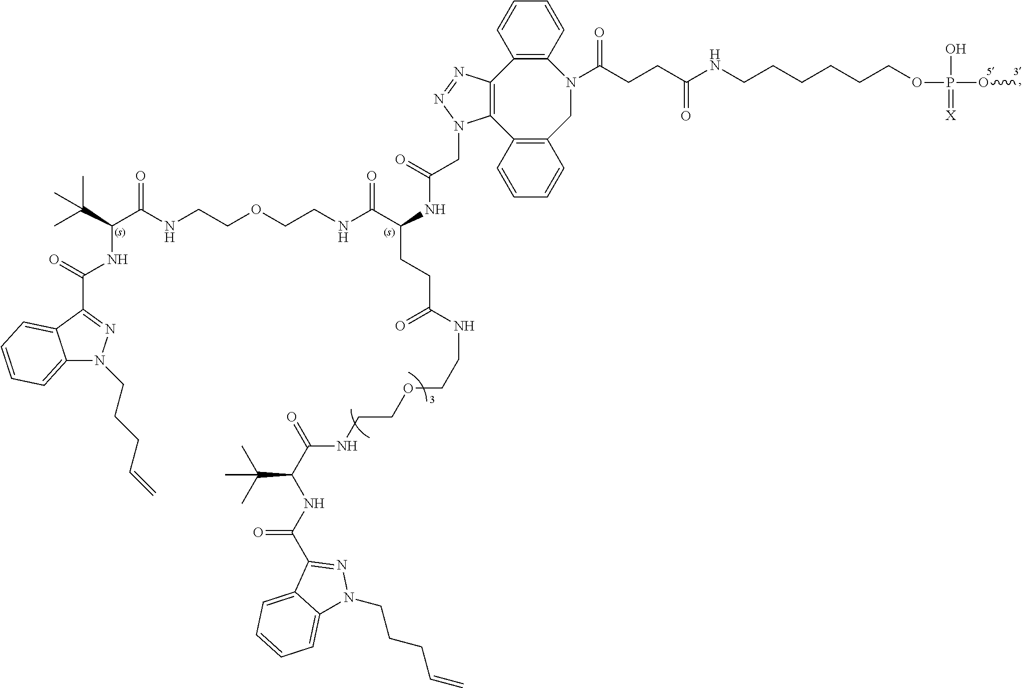

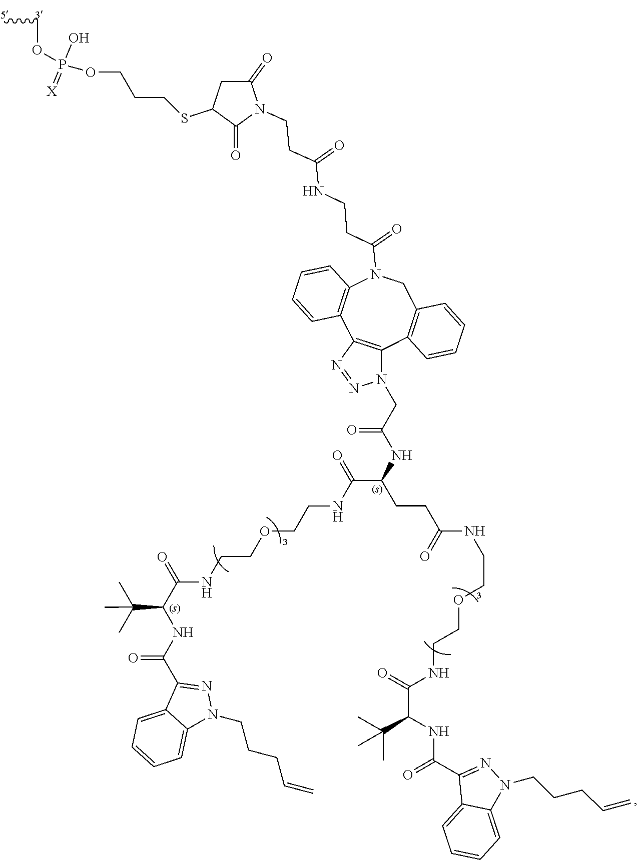

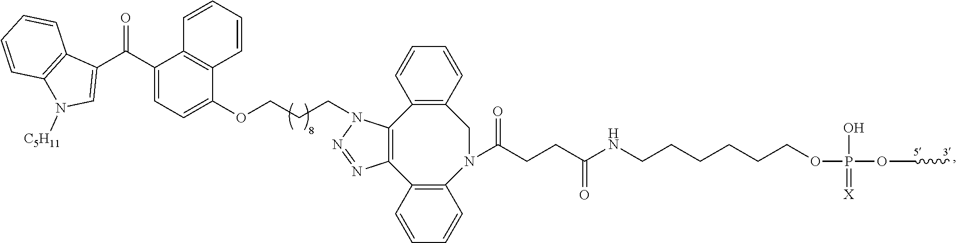

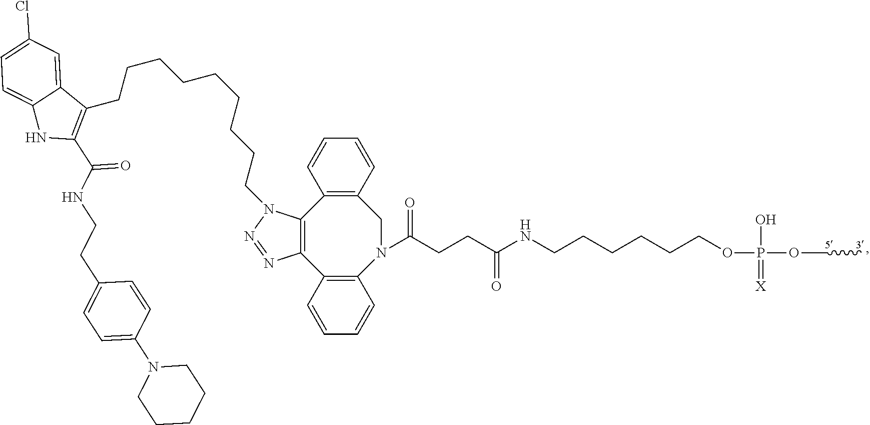

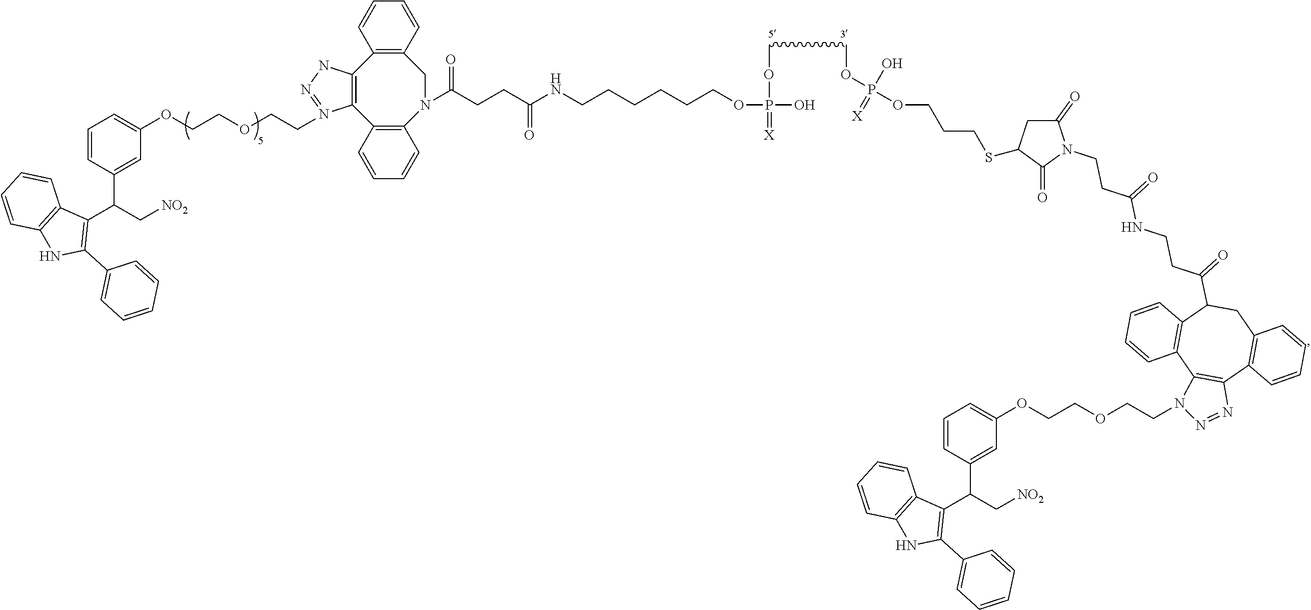

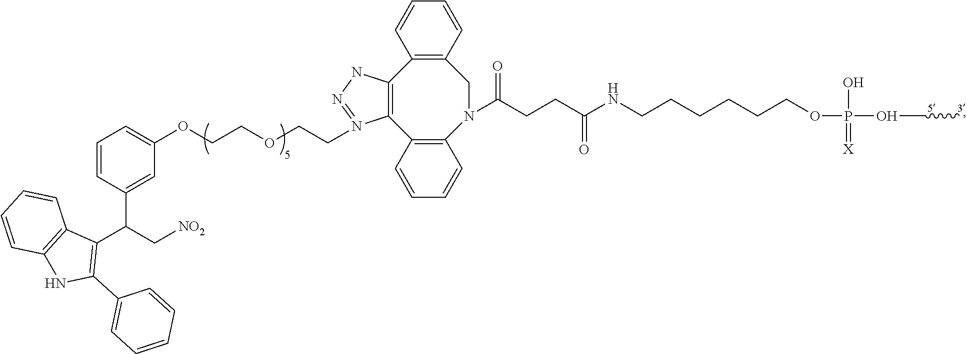

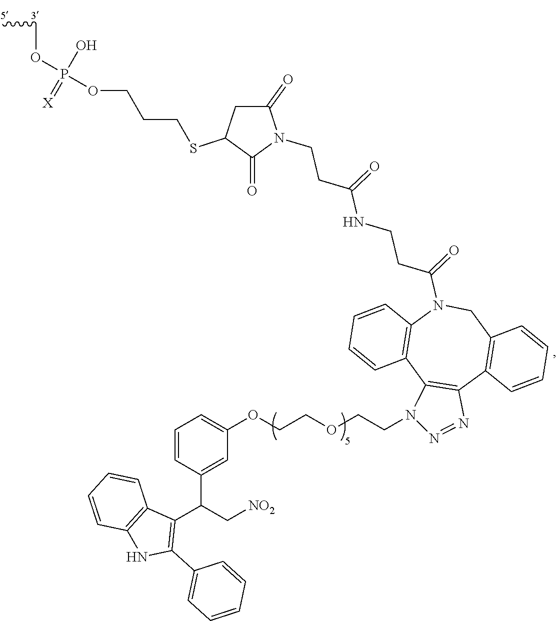

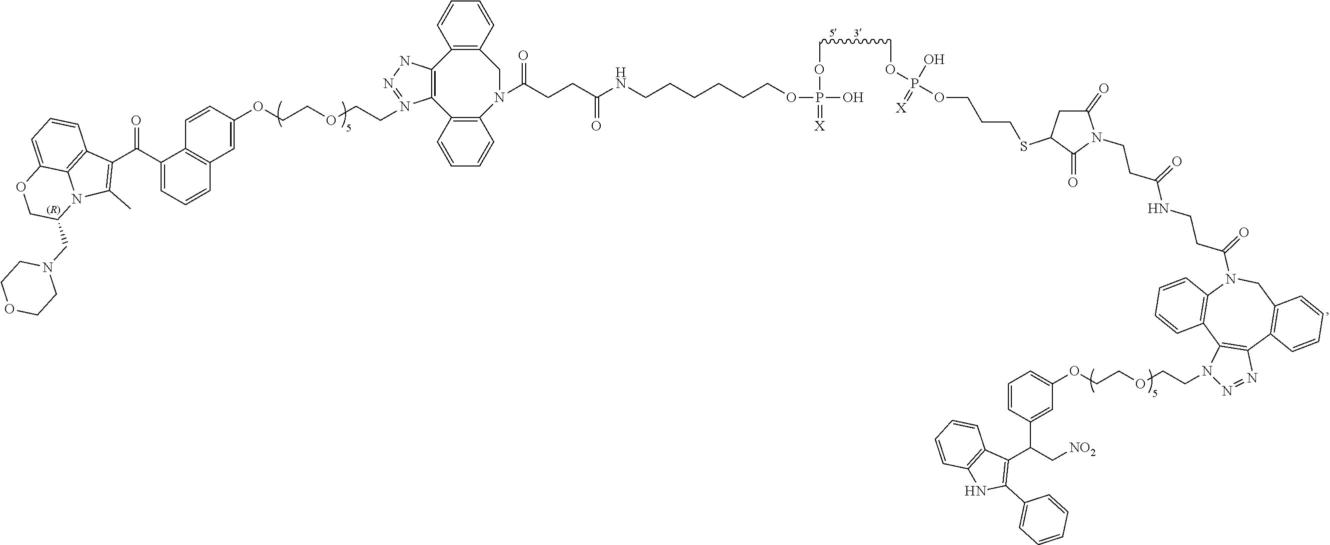

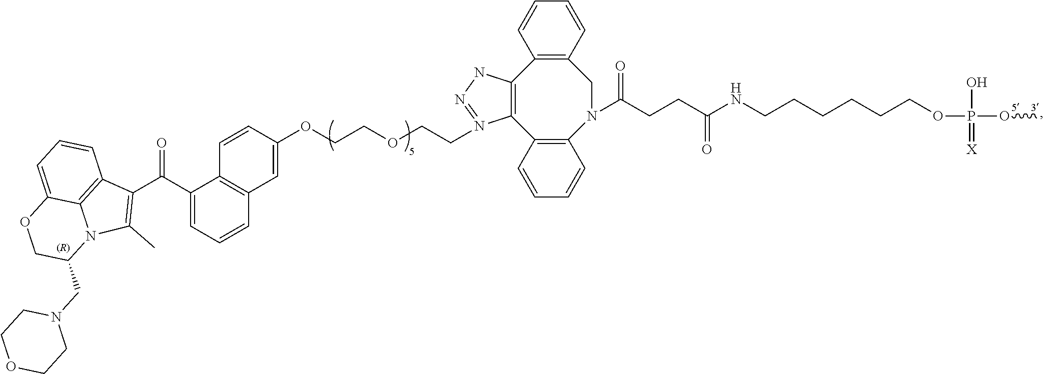

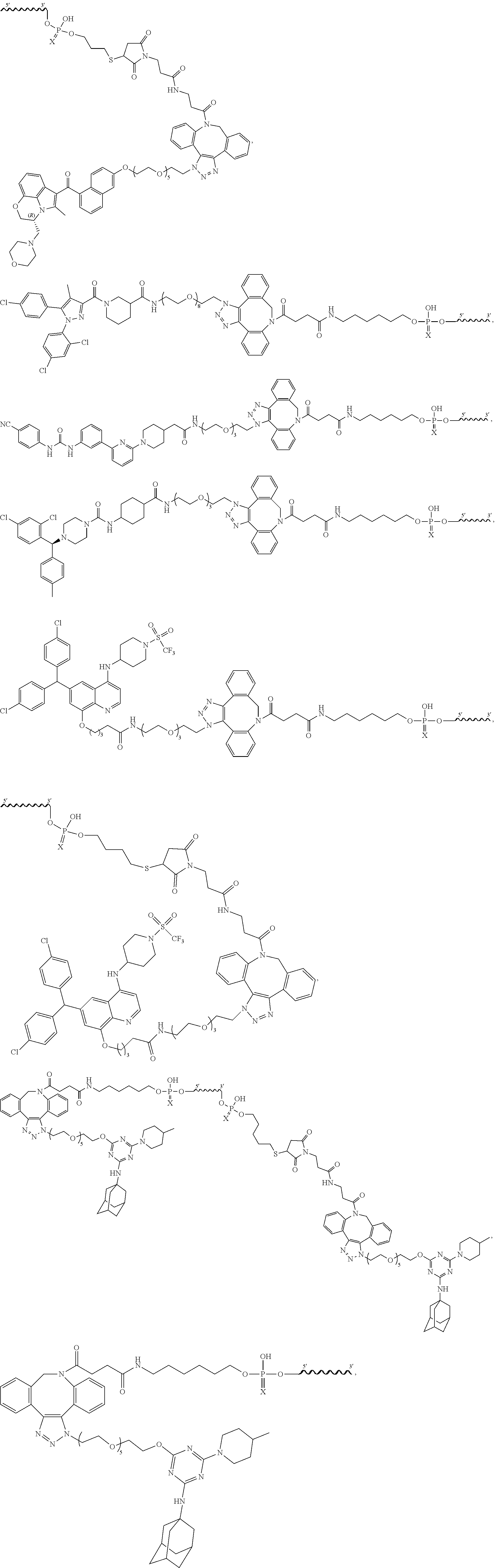

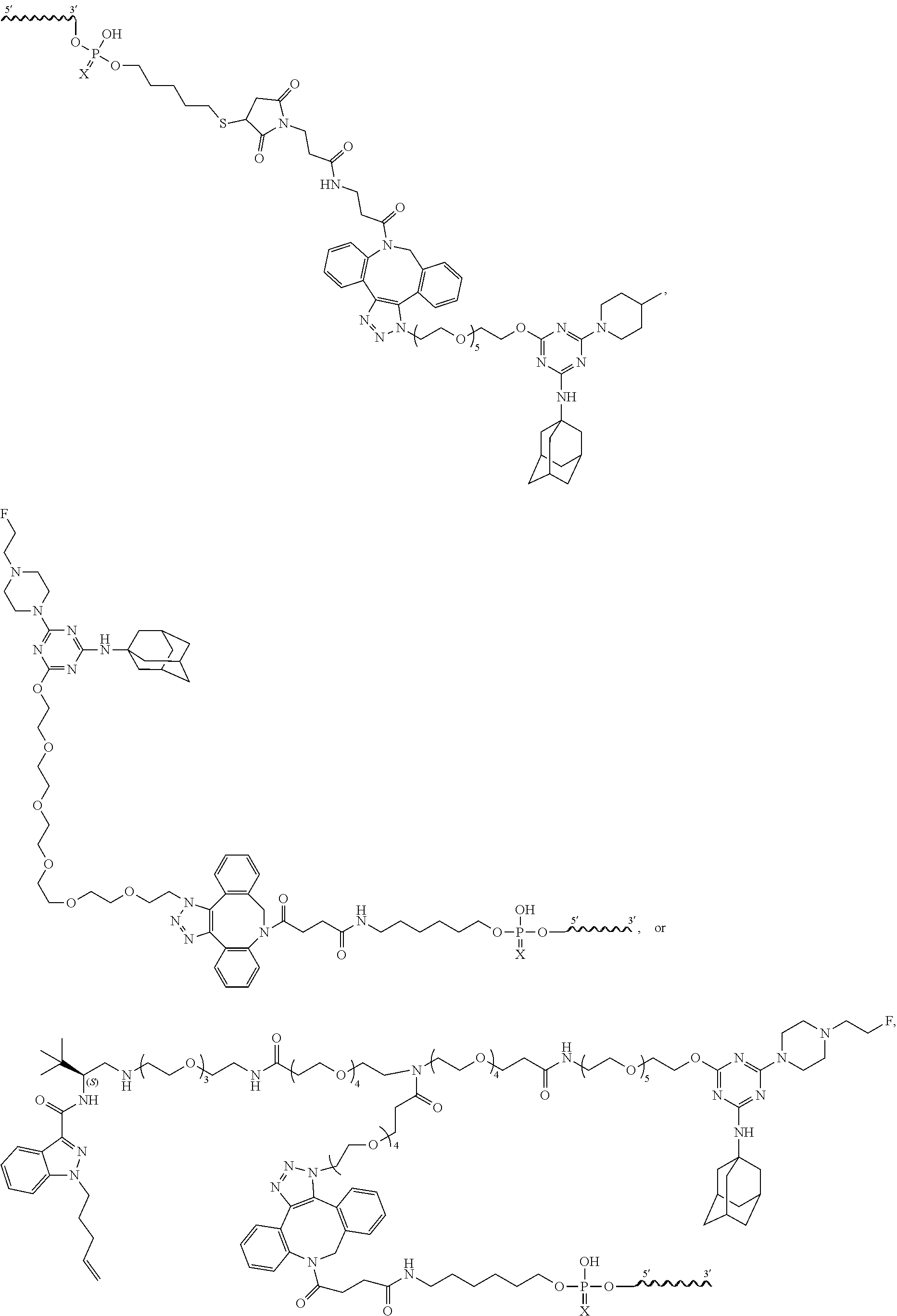

wherein:
[symbol] an oligonucleotide; and
X is O or S.

Embodiment 155. The compound, or a stereoisomer, tautomer, prodrug, or salt thereof, of any one of embodiments 112-154, wherein X is S.

Embodiment 156. The compound, or a stereoisomer, tautomer, prodrug, or salt thereof, of any one of embodiments 112-154, wherein X is O.

Embodiment 157. The compound, or a stereoisomer, tautomer, prodrug, or salt thereof, of any one of embodiments 1-156, wherein the salt is a potassium salt or a sodium salt.

Embodiment 158. A composition comprising a compound, or a salt thereof, of any one of embodiments 1-157, and a pharmaceutically acceptable excipient.

Embodiment 159. A method for delivering a therapeutic oligonucleotide to the brain of a subject, comprising administration of a compound, or a salt thereof, of any one of embodiments 1-157, or a composition of embodiment 158, to the subject.

Embodiment 160. The method of embodiment 159, wherein the therapeutic oligonucleotide is delivered to one or more brain regions selected from the group consisting of the striatum, the cerebellum, the brain stem, the hippocampus, the frontal cortex, and the spinal cord.

Embodiment 161. A method for treating or ameliorating a disease, disorder, or symptom thereof in a subject, comprising administration of a compound, or a salt thereof, of any one of embodiments 1-157, or a composition of embodiment 158, to the subject.

Embodiment 162. The method of embodiment 161, wherein the disease, disorder, or symptom thereof is a central nervous system (CNS) disease, disorder, or symptom thereof.

Embodiment 163. The method of embodiment 161 or 162, wherein the disease, disorder, or symptom thereof is Alzheimer's disease, or a symptom thereof.

Embodiment 164. The method of any one of embodiments 159-163, wherein the administration is intrathecal administration or intracerebroventricular (ICV) administration.

Embodiment 165. A precursor compound, or a stereoisomer, tautomer, or salt thereof, of any one of structural Formulae (A)-(M):

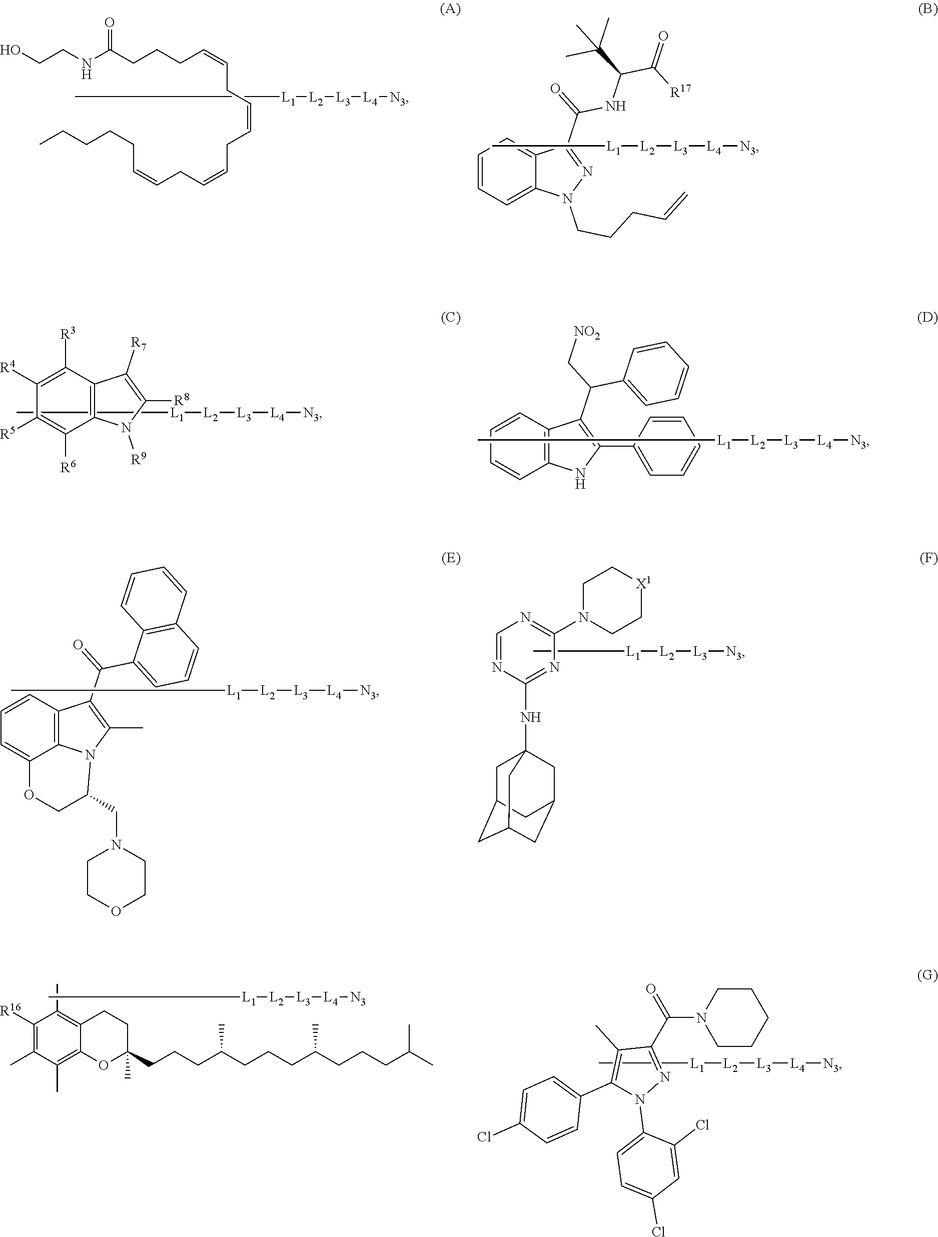

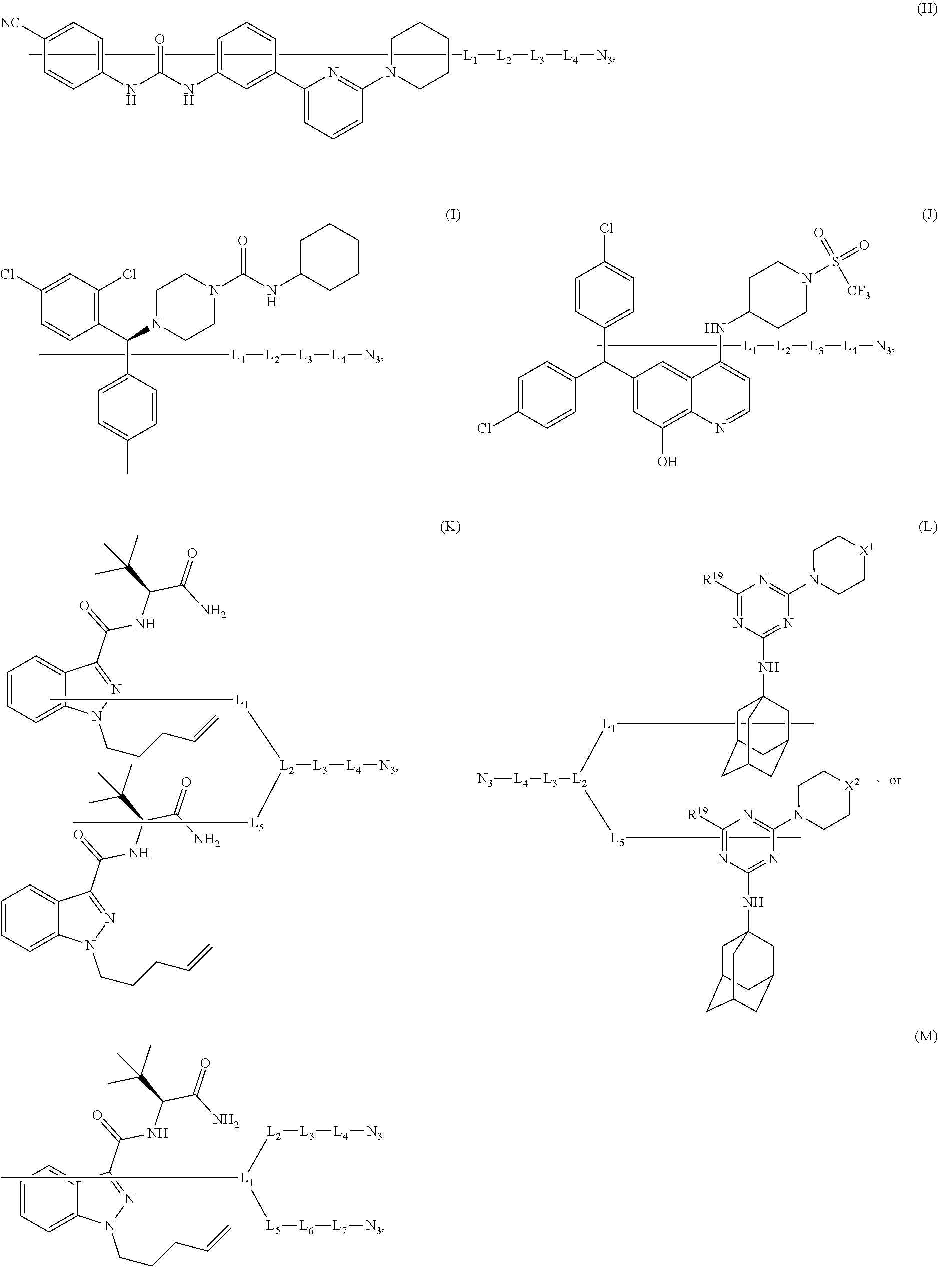

wherein:
each of L¹, L², L³, L⁴, L⁵, L⁶, and L⁷ is independently a linker, bond, or absent;
X¹ is NR¹⁰ or CR¹¹R¹²;
X² is NR¹³ or CR¹⁴R¹⁵;
R³, R⁴, R⁵, R⁶, and R⁸ are each independently hydrogen, halogen, optionally substituted alkyl, optionally substituted heteroalkyl, optionally substituted cycloalkyl, optionally substituted heterocycloalkyl, optionally substituted aryl, or optionally substituted heteroaryl;
R⁹ is hydrogen, optionally substituted alkyl, or optionally substituted heteroalkyl; or R⁶ and R⁹ substituents may be joined together form an optionally substituted heterocycloalkyl or optionally substituted heteroaryl;
R⁷ is hydrogen, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D};
—NR^{7B}C(O)OR^{7D}, —NR^{7B}OR^{7D}, optionally substituted alkyl, optionally substituted heteroalkyl, optionally substituted cycloalkyl, optionally substituted heterocycloalkyl, optionally substituted aryl, or optionally substituted heteroaryl;
R^{7A}, R^{7B}, R^{7C}, R^{7D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{7B} and R^{7C} substituents bonded to the same nitrogen atom may optionally be joined to form a substituted or unsubstituted heterocycloalkyl or substituted or unsubstituted heteroaryl;
n7 is 0, 1, 2, 3, or 4;
v7 is 1 or 2;
R¹⁰, R¹¹, R¹², R¹, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, —CN, —N₃, —NO₂, —NR^{16B}R^{16C}, —C(O)R^{16D}, —C(O)OR^{16D}, —C(O)NR^{16B}R^{16C}, —OR^{16A}, —NR^{16B}C(O)R^{16D}, optionally substituted alkyl, optionally substituted heteroalkyl, optionally substituted cycloalkyl, optionally substituted heterocycloalkyl, optionally substituted aryl, or optionally substituted heteroaryl;
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R^{18C}, —NHNR^{18B}R^{18C}, —ONR^{18B}R^{18C}, —NHC(O)NHNR^{18B}R^{18C}, —NHC(O)NR^{18B}R^{18C}, —NR^{18B}R^{18C}, —C(O)R^{18D}, —C(O)OR^{18D}, —C(O)NR^{18B}R^{18C}, —OR^{18A}, —NR^{18B}SO₂R^{18A}, —NR^{18B}C(O)R^{18D};
—NR^{18B}C(O)OR^{1D}, —NR^{18B}OR^{18D}, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D}, —NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R²⁰ is hydrogen, —SOₙ₂₀R^{20A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —NR^{20B}R^{20C}, —C(O)R^{20D}, —C(O)OR^{20D}, —C(O)NR^{20B}R^{18C}, —OR^{20A}, —NR^{20B}SO₂R^{20A}, —NR^{20B}C(O)R^{20D}; —NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is independently hydrogen, —SOₙ₂₁R^{21A}, —SOᵥ₂₁NR^{21B}R^{21C}, NHNR^{21B}R^{21C}, —ONR^{21B}R^{21C}, —NHC(O)NHNR^{21B}R^{21C}, —NHC(O)NR^{21B}R^{21C}, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
n19 and n21 are each independently 0, 1, 2, 3, or 4; and
v19 and v21 are each independently 1 or 2;
R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, R^{19D}, R^{20A}, R^{20B}, R^{20C}, R^{20D}, R^{21A}, R^{21B}, R^{21C}, and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{16B} and R^{16C}; R^{17B} and R^{17D}; R^{18B} and R^{18D}; R^{19B} and R^{19C}; and R^{20B} and R^{20C}; and R^{21B} and R^{21C} substituents bonded to the same nitrogen atom may optionally be joined to form a substituted or unsubstituted heterocycloalkyl or substituted or unsubstituted heteroaryl;
n16, n17, n18, n19, n20, and n21 are each independently 0, 1, 2, 3, or 4; and
v16, v17, v18, v19, v20, and v21 are each independently 1 or 2.

Embodiment 166. A method for making a compound, or a stereoisomer, tautomer, prodrug, or salt thereof, of any one of embodiments 1-157, comprising contacting the precursor compound of embodiment 165 with a compound of structural Formula (W) and/or (Q):

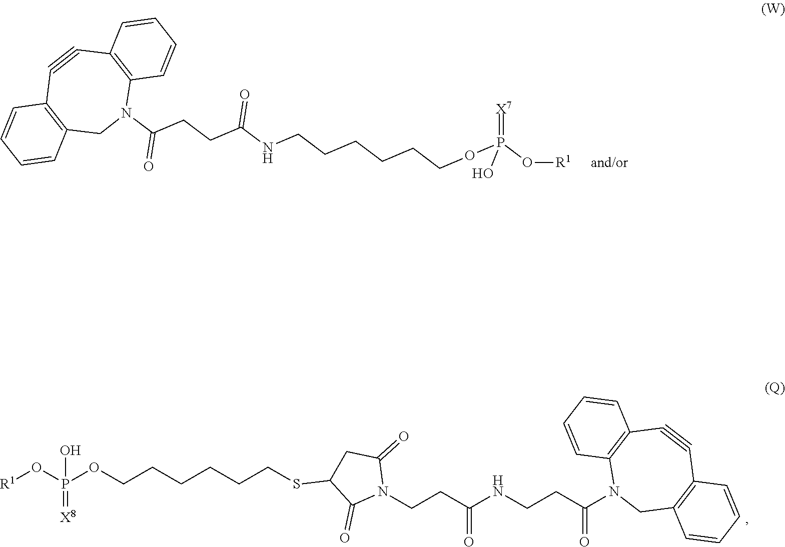

a salt thereof,
wherein X⁷ and X⁸ are each independently O or S.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

In order that the embodiments described herein may be more fully understood, the following examples are set forth. The examples described in this application are offered to illustrate the compounds, compositions, and methods provided herein and are not to be construed in any way as limiting their scope.

### General Experimental Procedures

Definitions of variables in the structures in schemes herein are commensurate with those of corresponding positions in the formulae delineated herein.

### Common Abbreviations

ACN acetonitrile
br broad
CDI carbonyldiimidazole
d doublet
DCM dichloromethane
dd doublet of doublets
dba dibenzylideneacetone
DBCO Azadibenzocyclooctyne
DFAA difluoroacetic anhydride
DIPEA or DIEA diisopropylethylamine
DMF dimethylformamide
DMSO dimethyl sulfoxide
dppf 1,1′-ferrocenediyl-bis(diphenylphosphine)
EDCI 1-ethyl-3-(3-dimethylaminopropyl)carbodiimide
EtOAc ethyl acetate
h hour(s)
HRMS high resolution mass spectrometry
HPLC high performance liquid chromatography
LCMS liquid chromatography and mass spectrometry
MS mass spectrometry
MW microwave
MW (g/mol) molecular weight (g/mol)
m multiplet
MeOH methanol
min minutes
mL milliliter(s)
MWCO molecular weight cut off
m/z mass to charge ratio
NMP N-methyl-2-pyrrolidone
NMR nuclear magnetic resonance
ppm parts per million
rt or RT room temperature
s singlet
t triplet
TFAA trifluoroacetic anhydride
TLC thin layer chromatography
TCEP Tris(2-carboxyethyl)phosphine hydrochloride

### Example 1A-1B: Synthesis of CB₁ Ligand-Conjugated Oligonucleotides

Exemplary compounds falling within the scope of the present disclosure can be synthesized according to the following procedures:

### Example 1A

### General Procedure I: 5′-Conjugated Sense Strand

Step 1: To an aqueous solution of 5′-amine functionalized sense strand (I) was added 10% V/V 1M Sodium Phosphate buffer (pH=7) and 20%-50% V/V CH₃CN. A solution of DBCO—NHS (II) (1.5-3 eq) in DMSO or CH₃CN was added to the reaction. The reaction is monitored by LCMS and HPLC. Upon completion, precipitate was removed using centrifugation and the aqueous solution was purified by reverse phase HPLC. The product fractions were combined and dried by lyophilization. The dried N-DBCO modified sense strand (III) was reconstituted in RNase free water for step 2.

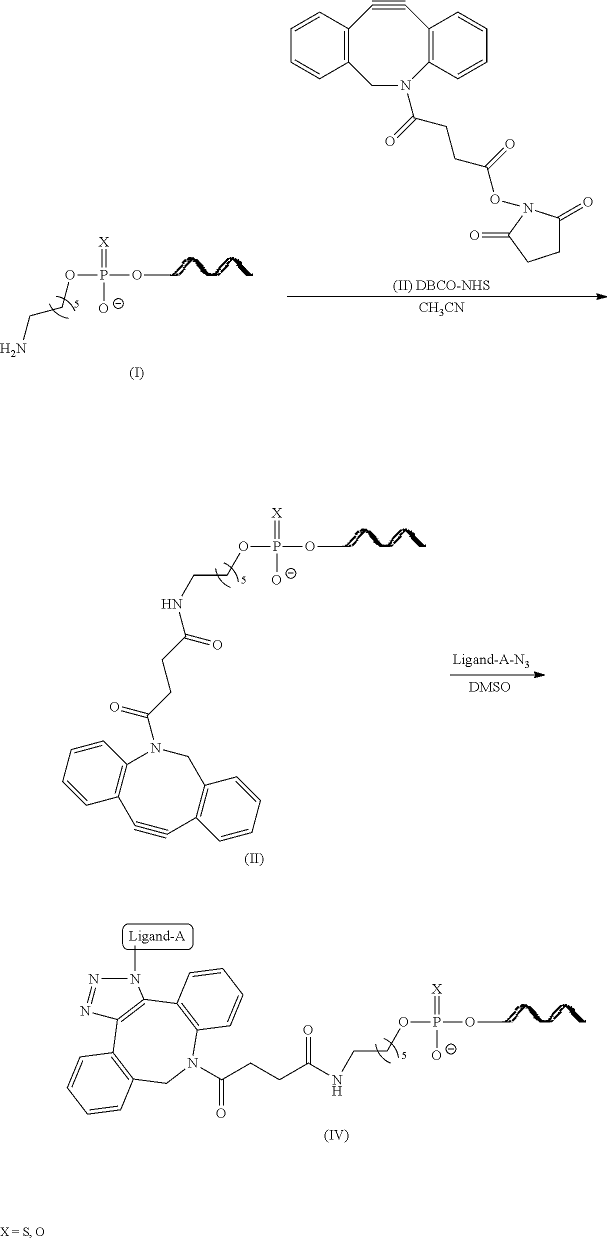

Step 2: To a solution of 5′-DBCO modified sense strand (III) (1 eq) was added a solution of ligand-A-N₃ (2 eq) in DMSO or THF. The reaction was monitored by HPLC and LCMS. Upon completion, the 5′-conjugated sense strand (IV) was purified by reverse phase HPLC or molecular weight cut-off with Amicon® Ultra-15 Centrifugal filter (3K, 5 times).

### General Procedure II Type A: Bis-Conjugated Sense Strand

Step 1: To an aqueous solution of 5′ amine functionalized sense strand (I) was added 10% V/V 1M Sodium Phosphate buffer (pH=7) and 20%-50% V/V CH₃CN. A solution of DBCO—NHS (II) (1.5-3 eq) in DMSO or CH₃CN was added to the reaction. The reaction was monitored by LCMS and HPLC. Upon completion, precipitate was removed by centrifugation and the aqueous solution was purified by reverse phase HPLC. The product fractions were combined and dried by lyophilization. The 5′-DBCO modified sense strand (III) was reconstituted in RNase free water for step 2.

Step 2: To an aqueous solution of 5′-DBCO modified, 3′-(C6-SS—C6)-mC functionalized sense strand (III) was added 10% V/V 1M Sodium Phosphate buffer (pH=7). Tris(2-carboxyethyl)phosphine hydrochloride (TCEP) (25 eq) was dissolved in H2O and the PH of the solution was adjusted to 7 using 10 M NaOH. The aqueous TCEP solution was added to the solution of sense strand (Ill). The reaction was monitored by HPLC and LCMS. Upon completion, excess TCEP was removed by MWCO with 100 mM sodium phosphate buffer (pH=7) (3×). To the disulfide-reduced sense strand solution was added a solution of DBCO-MAL (V) (3 eq) in DMSO. The reaction was monitored by LCMS and HPLC. Upon completion, the solid was removed by centrifugation and the solution was purified by reverse phase HPLC and dried by lyophilization. The dried bis-DBCO modified sense strand (VI) was reconstituted in RNase free water for step 3.

Step 3: To a solution of 5′-, 3′-DBCO functionalized sense strand (VI) (1 eq) was added a solution of ligand-A-N₃ (3 eq) in DMSO or THE. The reaction was monitored by HPLC and LCMS. Upon completion, the 5′-, 3′-bis-conjugated sense strand (VII) was purified by reverse phase HPLC or molecular weight cut-off with Amicon© Ultra-15 Centrifugal filter (3K, 5 times). The product was confirmed by HPLC and LCMS.

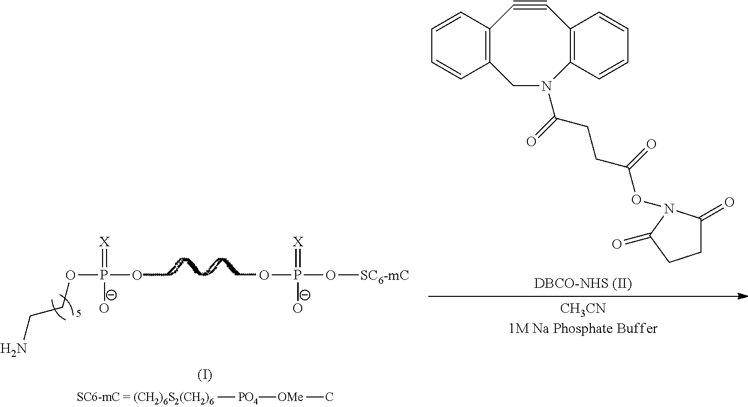

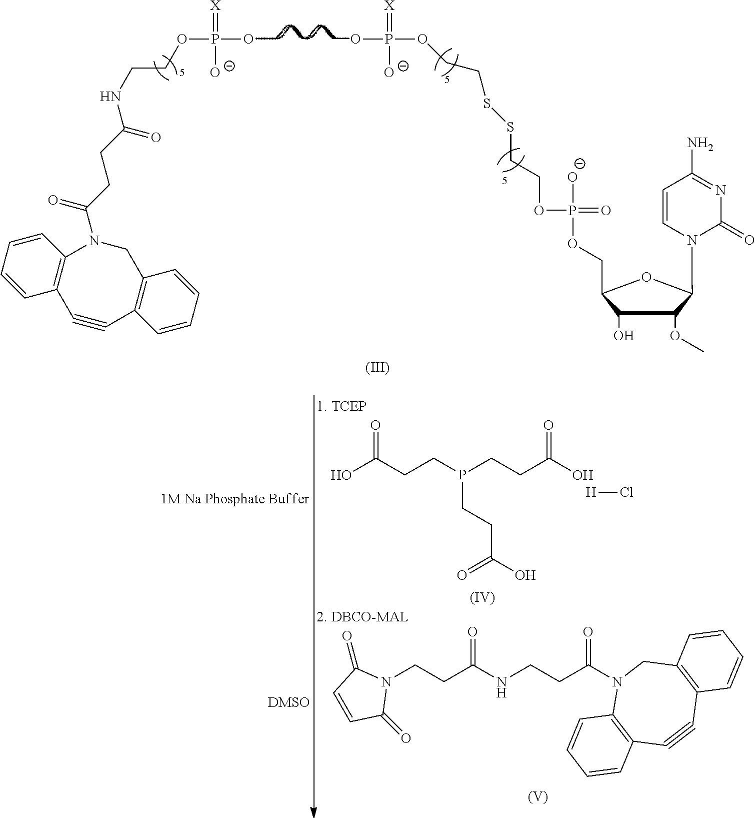

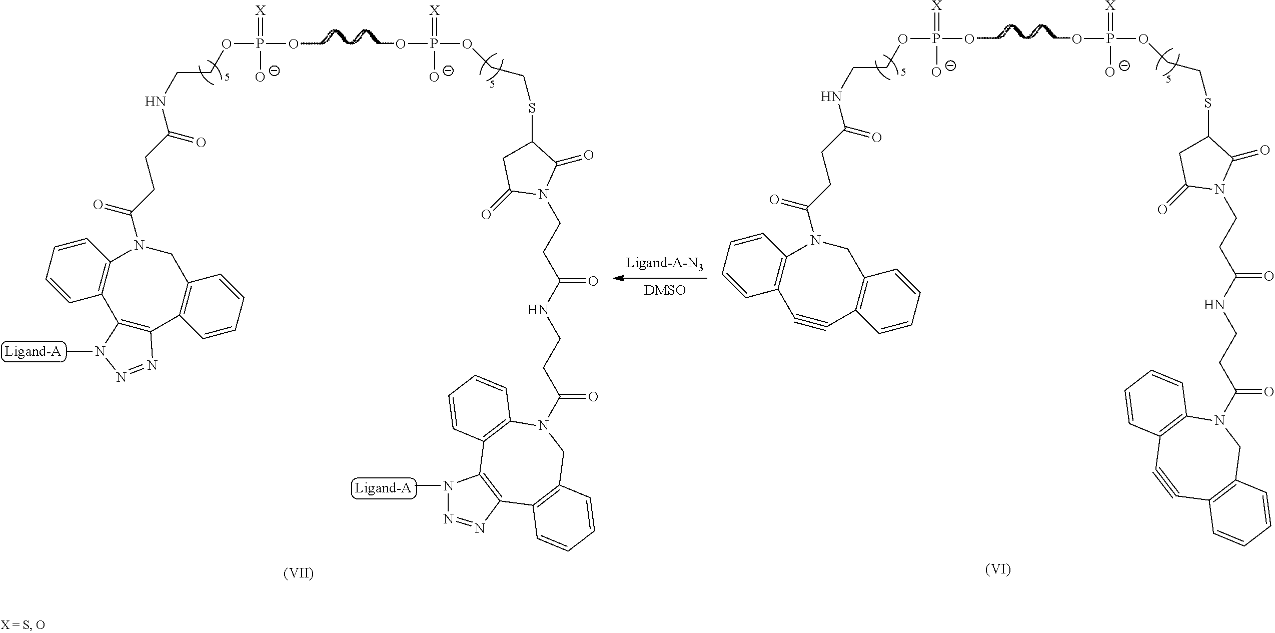

### General Procedure II Type B: 3′,5′-Conjugated Sense Strand

Step 1: To an aqueous solution of 5′-amine, 3′-(C6-SS—C6)-mC functionalized sense strand (I) was added 10% V/V 1M Sodium Phosphate buffer (pH=7) and 20%-50% V/V CH₃CN. A solution of DBCO—NHS (II) (1.5-3 eq) in DMSO or CH₃CN was added to the reaction. The reaction was monitored by LCMS and HPLC. Upon completion, precipitate was removed by centrifugation and the aqueous solution was purified by reverse phase HPLC. The combined product fractions were collected and dried by lyophilization. The 5′-DBCO modified sense strand (III) was reconstituted in RNase free water for step 2.

Step 2: To an aqueous solution of 5′-DBCO modified sense strand (III) (1 eq) was added a solution of ligand-A-N₃ (2 eq) in DMSO. The reaction is monitored by HPLC and LCMS. Upon completion, the 5′-conjugated sense strand (IV) was purified by reverse phase HPLC or molecular weight cut-off with Amicon® Ultra-15 Centrifugal filter (3K, 5 times).

Step 3: To a solution of 5′-conjugated, 3′-(C6-SS—C6)-mC functionalized sense strand (IV) (1 eq) in H₂O was added 10% V/V 1M sodium phosphate buffer (pH=7). TCEP (V) (20 eq) was dissolved in H₂O and the PH of the solution was adjusted to 7 using 10 M NaOH. The aqueous TCEP solution was added to the solution of sense strand (IV). The reaction was monitored by HPLC and LCMS. Upon completion, excess TCEP was removed with 100 mM sodium phosphate buffer (pH=7) using MWCO (3×). A solution of DBCO-MAL (VI) (3 eq) in DMSO was added to the disulfide-reduced sense strand solution. The reaction was monitored by HPLC and LCMS. Upon completion, the aqueous solution was purified by reverse phase HPLC. The product fractions were collected and dried by lyophilization. The 5′-conjugated, 3′-DBCO modified sense strand (VII) was reconstituted in 100 mM Sodium Phosphate buffer for the next step.

Step 4: To an aqueous solution of 5′-conjugated, 3′-DBCO functionalized sense strand (VII) (1 eq) was added a solution of ligand-B—N₃ (2 eq) in DMSO. The reaction was monitored by HPLC and LCMS. Upon completion, the 5′-, 3′-conjugated sense strand (VIII) was purified by reverse phase HPLC or molecular weight cut-off with Amicon® Ultra-15 Centrifugal filter (3K, 5 times).

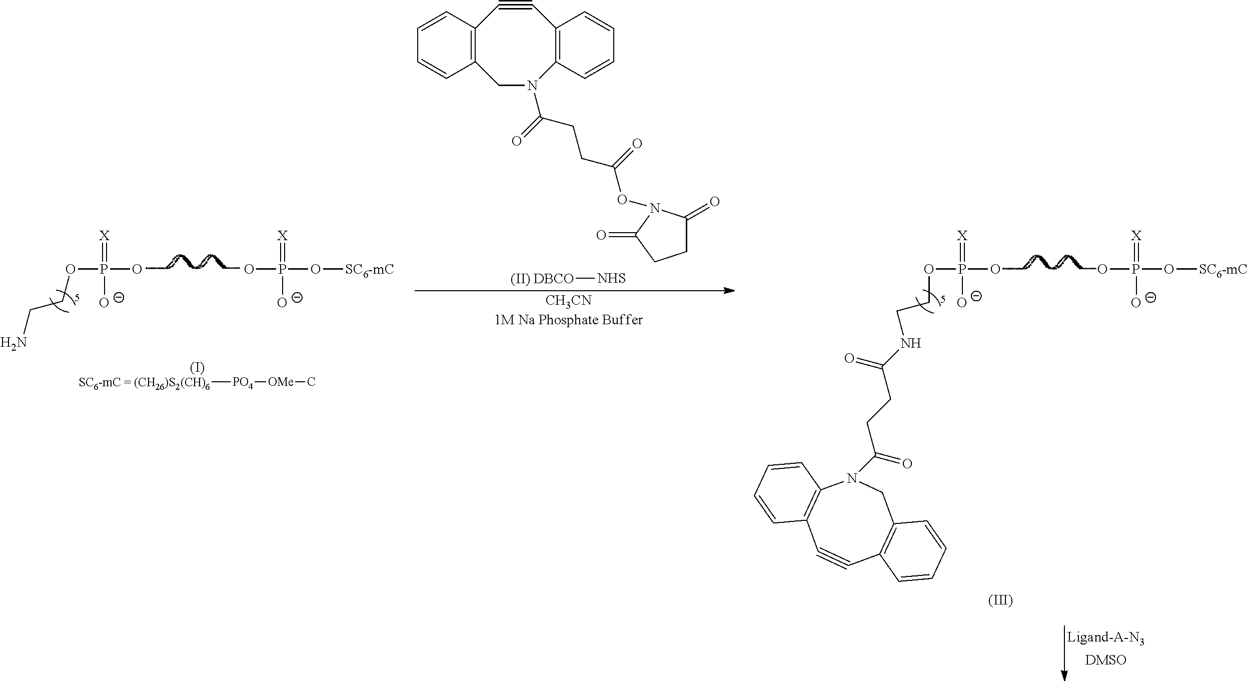

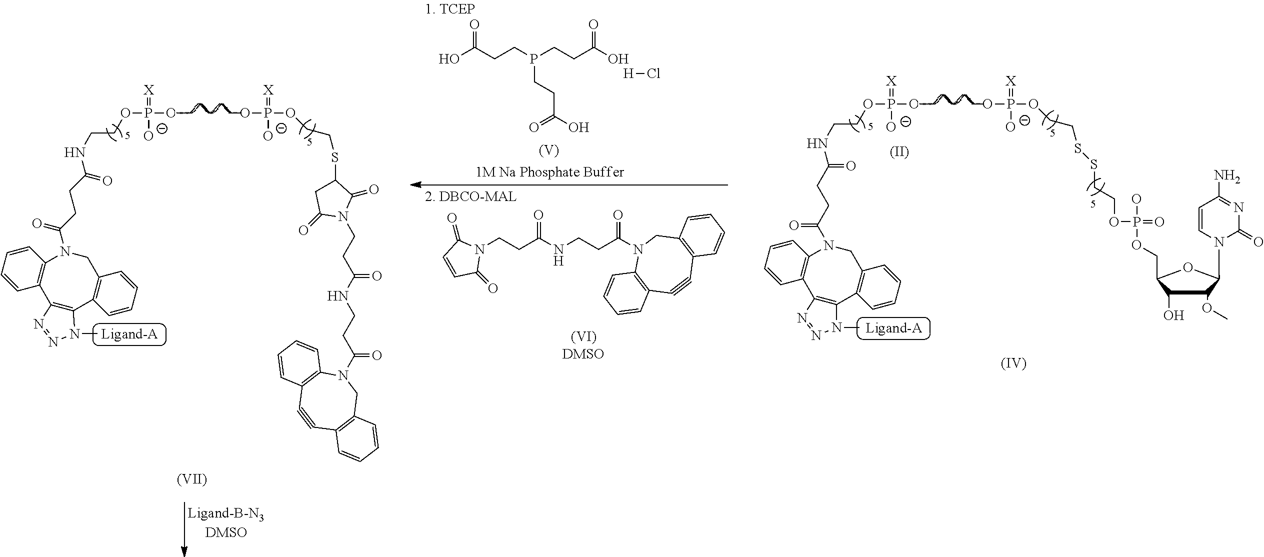

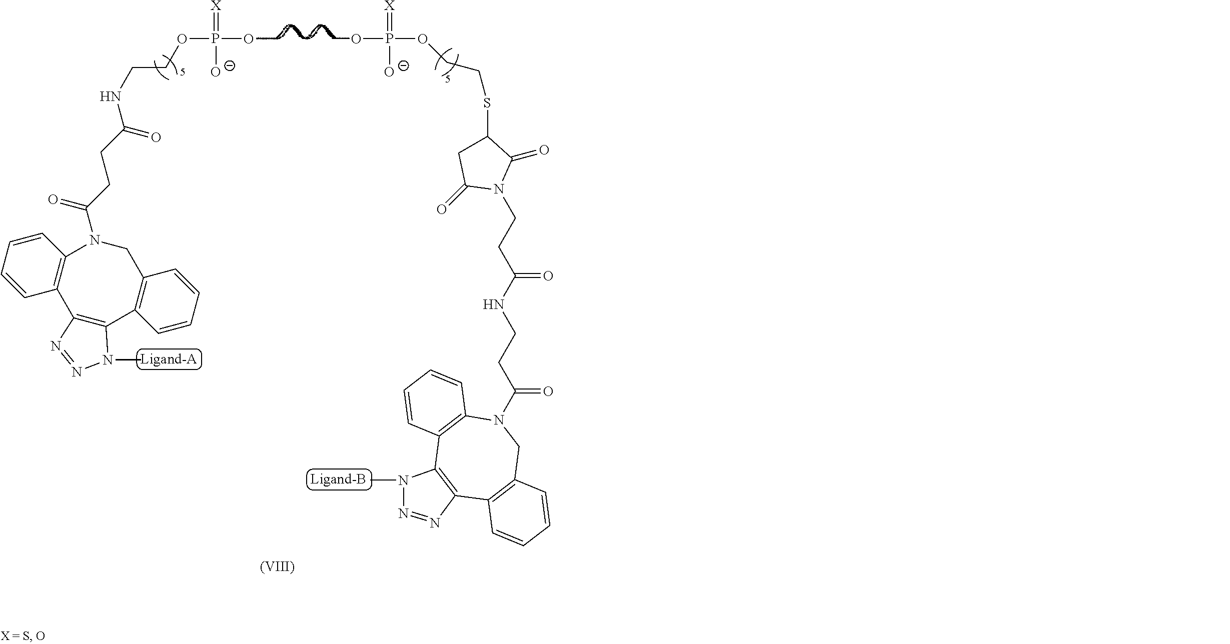

### General Procedure III: 3′-Conjugated Sense Strand

Step 1: To an aqueous solution of 3′-(C6-SS—C6)-mC functionalized sense strand (I) was added 10% V/V 1M Sodium Phosphate buffer (pH=7). TCEP (II) (20 eq) was dissolved in H₂O and the PH of the solution was adjusted to 7 using 10 M NaOH. The aqueous TCEP solution was added to the solution of sense strand (1) The reaction was monitored by HPLC and LCMS. Upon completion, excess TCEP was removed by MWCO with 100 mM sodium phosphate buffer (pH=7) (3×). To the disulfide-reduced sense strand solution was added a solution of DBCO-MAL (III) (3 eq) in DMSO. The reaction was monitored by LCMS and HPLC. Upon completion, the solid was removed by centrifugation and the solution was purified by reverse phase HPLC. The product fractions were collected and dried by lyophilization. The dried 3′-DBCO modified sense strand (IV) was reconstituted in 100 mM Sodium Phosphate buffer for step 3.

Step 3: To an aqueous solution of 3′-DBCO functionalized sense strand (IV) (1 eq) was added a solution of ligand-A-N₃ (3 eq) in DMSO. The reaction is monitored by HPLC and LCMS. Upon completion, the 3′-conjugated sense strand (V) was purified by reverse phase HPLC or molecular weight cut-off with Amicon® Ultra-15 Centrifugal filter (3K, 5×). The product was confirmed by HPLC and LCMS.

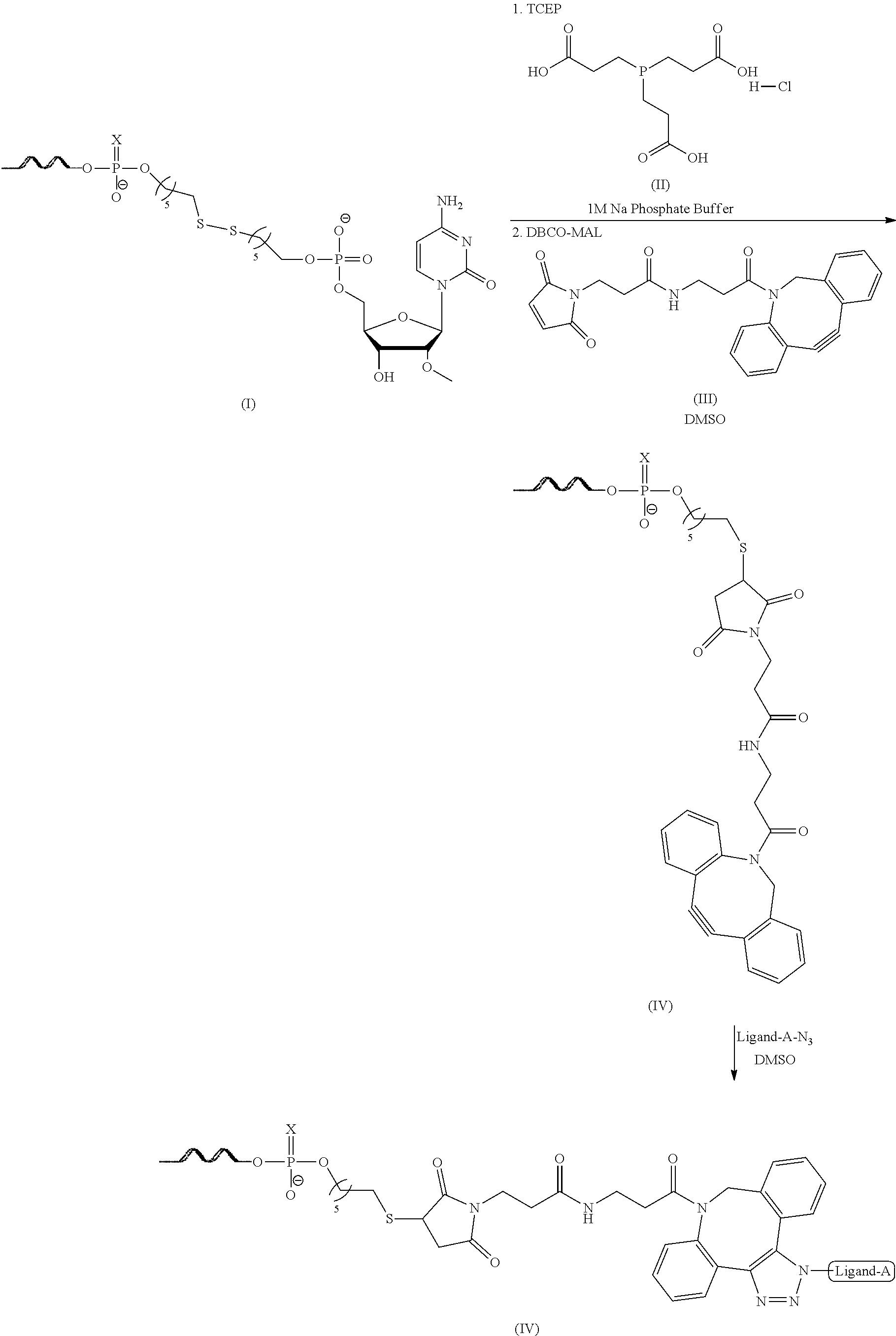

### Example 1B

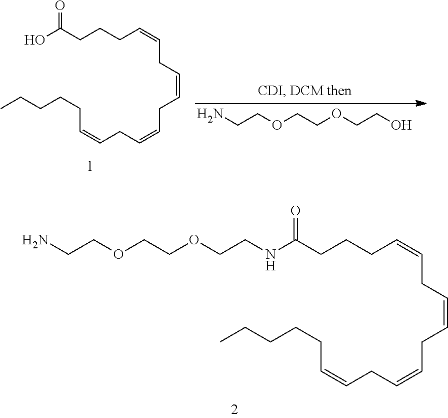 [SMILES: CCCCC/C=C\C/C=C\C/C=C\C/C=C\CCCC(=O)O ; NCCOCCOCCO ; CCCCC/C=C\C/C=C\C/C=C\C/C=C\CCCC(=O)NCCOCCOCCN]

To a stirred solution of arachidonic acid 1 (1 g, 3.28 mmol, 1 eq) in DCM (20.0 ml) at rt was added CDI (586 mg, 3.61 mmol, 1.1 eq), and the reaction mixture was stirred at room temperature for 1.5 h. The amine (980 mg, 0.91 ml, 6.57 mmol, 2 eq) was then added dropwise, and the mixture was stirred at it for another 2 h. The mixture was diluted with DCM and washed three times with water and then brine. The organic layers were dried over Na₂SO₄, filtered, and then concentrated. The residue obtained was purified by silica-gel column chromatography using a gradient 0-10% methanol in ethyl acetate to afford the desired amide 2 in 61% yield (867 mg). MS ESI+ m/z=436.3 [MH]+, 458.1 [MNa]+.

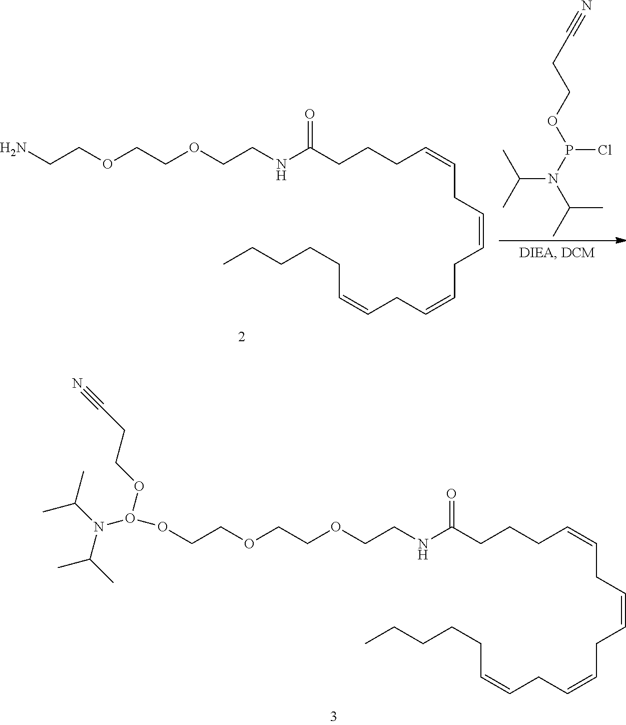

To a stirred solution of amide 2 (680 mg, 1.56 mmol, 1 eq) in DCM (36.0 ml) at rt was added DIEA (1.76 ml, 10.14 mmol, 6.5 eq) followed by 2-cyanoethyl-N,N′-diisopropylchlorophosphoramidite (0.38 ml, 1.72 mmol, 1.1 eq) dropwise, and the reaction mixture was stirred at room temperature for 1.5 h. The reaction was quenched with aq. saturated NaHCO₃. The product was extracted twice with DCM, and the organic layers were combined, washed with aq. saturated NaHCO₃, washed with brine, dried over N₂SO₄, filtered, and then concentrated. The residue obtained was purified by silica-gel column chromatography using a gradient 0-80% ethyl acetate in hexanes containing 2% Et₃N as an additive to afford the desired product 3 in 64% yield (632 mg). MS ESI+ m/z=636.3 [MH]+, 658.3 [MNa]+.

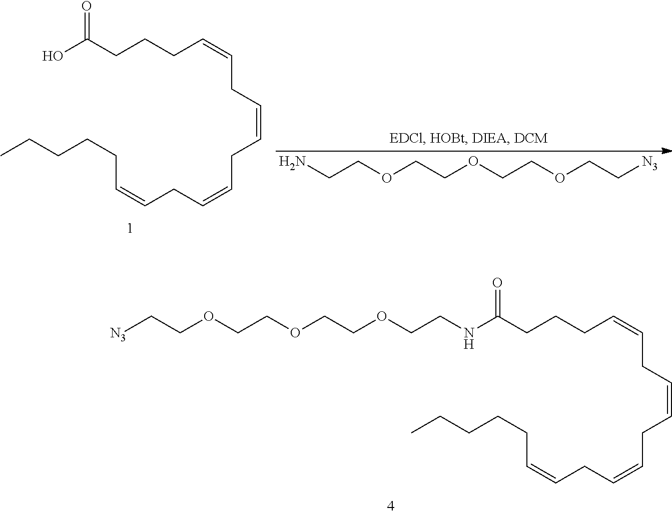

To a solution of arachidonic acid (1 g, 3.28 mmol, 1 eq) in DCM (20 ml) at 0° C. were successively added HOBt (0.65 g, 3.61 mmol, 1.1 eq) and EDCI (816 mg, 4.27 mmol, 1.3 eq). The cooling bath was removed, and the mixture was stirred for 1 h at rt. Azido peg3 amine (932 mg, 4.27 mmol, 1.3 eq) was added dropwise, followed by DIEA (0.544 ml, 3.94 mmol, 1.2 eq), and the mixture was stirred overnight at rt. The mixture was diluted with DCM and washed with water then brine. The organic layer was dried, filtered, concentrated, and then purified by silica-gel column chromatography using a gradient 0-5% methanol in ethyl acetate to afford the desired product 4 in 78% yield (1.29 g) as an oil. ESI+ m/z 527.3 [MNa]+.

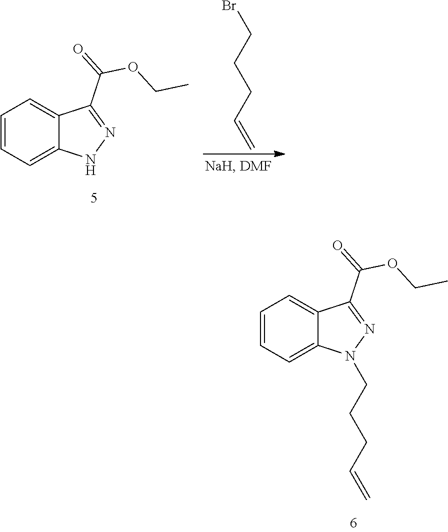

To a solution of Indazole 5 (3 g, 15.773 mmol, 1 eq) in DMF (31 mL) at 0° C. was added NaH (0.757 g, 18.927 mmol, 1.2 eq) portionwise. The mixture was stirred for 1 h at rt, cooled to 0° C. again, and then treated dropwise with 5-bromo-1-pentene (3.526 g, 23.659 mmol, 1.5 eq). The mixture was stirred at ambient temperature for 48 h then poured onto water and extracted with EtOAc. The combined organic layers were washed with water and brine, then dried, filtered, and concentrated. The residue obtained was purified by silica-gel column chromatography using a gradient 0-5% EtOAc in hexanes followed by 5-10% to afford the desired product 6 in 47% yield (1.91 g). MS ESI m/z=281.0 [MNa]+.

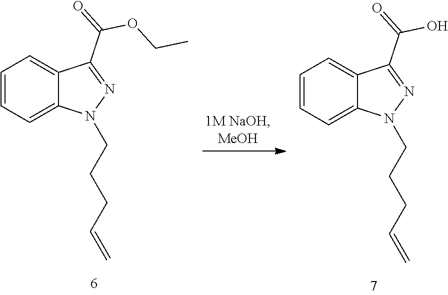 [SMILES: C=CCCCn1nc(C(=O)OCC)c2ccccc21 ; C=CCCCn1nc(C(=O)O)c2ccccc21]

To a solution of ester 6 (1.74 g, 6.73 mmol) in MeOH (81 mL) at 0° C. was added a 1N NaOH solution (14 mL) dropwise. The cooling bath was removed, and the mixture was stirred for 16 h at rt. The mixture was concentrated, and the residue obtained was taken up in water (50 ml) and then acidified to pH 3-4 by addition of 1M HCl at 0° C. The product was extracted with ethyl acetate, and the organic phases were combined, washed with brine, dried, filtered, and then concentrated to afford the desired product 7, which was used without further purification (1.27 g, 90%). MS ESI+ m/z=231.3 [MH]+.

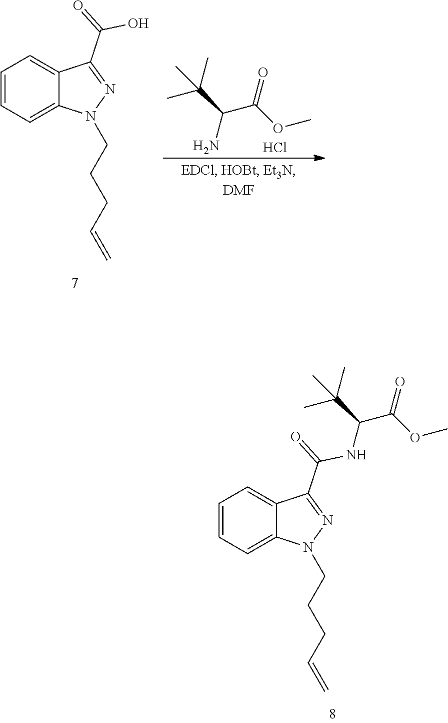

To a mixture of acid 7 (426 mg, 1.850 mmol), and L-tert-Leucine methyl ester hydrochloride (370 mg, 2.0 mmol, 1.1 eq), in DMF (6.8 mL) were successively added HOBt (0.344 g, 2.035 mmol, 1.1 eq), and EDC·HCl (532 mg, 2.77 mmol, 1.5 eq). Et₃N (842 mg, 8.32 mmol, 1.16 ml, 3.5 eq) was added dropwise and the mixture was stirred at rt for 24 h. The mixture was poured onto water and extracted with EtOAc. The combined organic layers were washed with water and brine and then dried, filtered, and concentrated. The residue obtained was purified by silica-gel column chromatography using a gradient 0-100% ethyl acetate in hexanes to afford the desired product 8 in 93% yield (616 mg). MS ESI+ m/z=358.5 [MH]+.

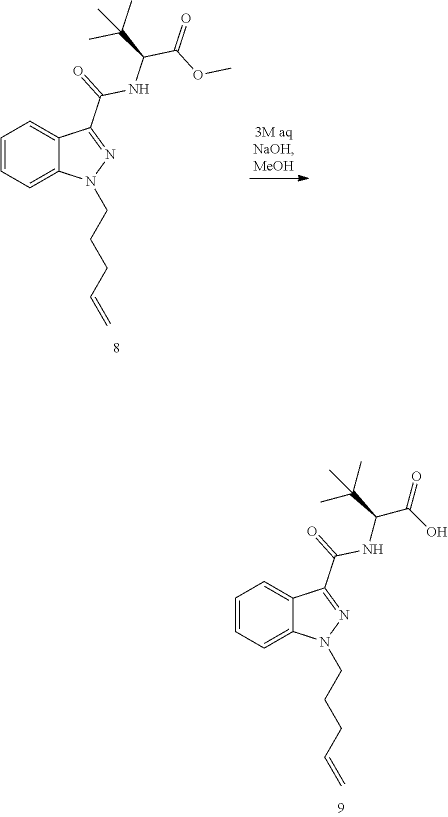 [SMILES: C=CCCCn1nc(C(=O)N[C@H](C(=O)OC)C(C)(C)C)c2ccccc21 ; C=CCCCn1nc(C(=O)N[C@H](C(=O)O)C(C)(C)C)c2ccccc21]

To a solution of ester 8 (595 mg, 1.66 mmol) in MeOH (12 mL) was added 3 M aq. NaOH (3.6 mL) was added dropwise at 0° C. The cooling bath was removed after 10 mins, and the mixture was stirred at ambient temperature overnight. The reaction was concentrated, and the residue was dissolved in water (20 ml) and then acidified by addition of 1M HCl dropwise at 0° C. until pH 3-4. The aqueous mixture was extracted with ethyl acetate, and the combined extracts were washed with brine, dried, filtered, and concentrated to afford the desired carboxylic acid 9 in 94% yield (539 mg). MS ESI+ m/z=344.4 [MH]+.

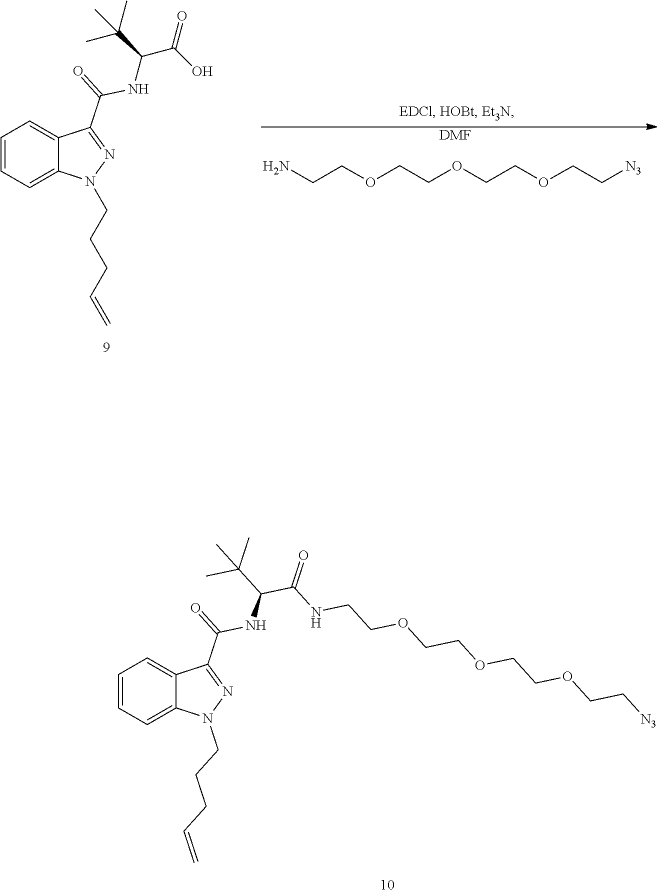

To a solution of Indazole 9 (0.15 g, 0.437 mmol, 1 eq) and HOBt (0.096 g, 0.568 mmol, 1.3 eq) in DMF (2.0 mL) was added EDCI. HCl (0.126 g, 0.655 mmol, 1.5 eq) followed by Et₃N (0.221 g, 2.184 mmol, 0.299 ml, 5 eq) at 0° C. The cooling bath was removed, and the mixture was stirred for 30 mins at rt. Azido peg3 amine (0.143 g, 0.655 mmol, 1.5 eq) was added in DMF (0.3 ml) dropwise and the mixture was stirred at rt for 1 h. The mixture was directly added onto a C18 column and purified by reverse-phase column chromatography using a gradient 0-50% acetonitrile in water (+0.1% formic acid) to afford the desired product 10 in 51% yield (122 mg). MS ESI+ m/z=544.5 [MH]+.

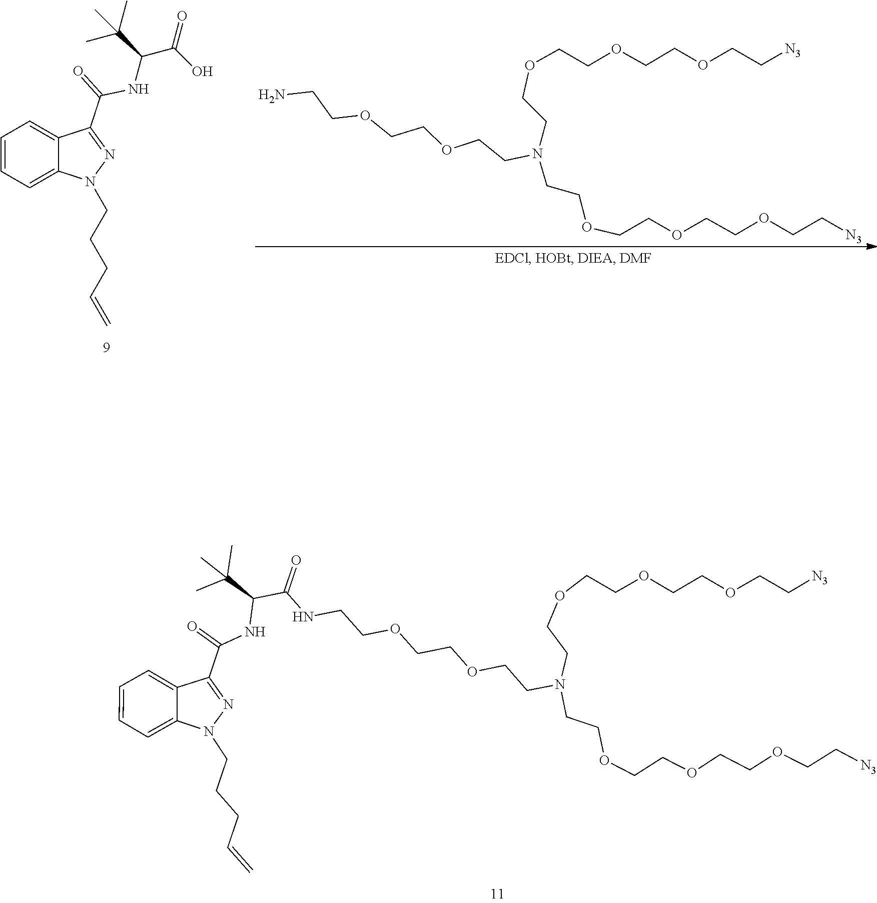 [SMILES: C=CCCCn1nc(C(=O)N[C@H](C(=O)O)C(C)(C)C)c2ccccc21 ; [N-]=[N+]=NCCOCCOCCOCCN(CCOCCOCCN)CCOCCOCCOCCN=[N+]=[N-] ; C=CCCCn1nc(C(=O)N[C@H](C(=O)NCCOCCOCCN(CCOCCOCCOCCN=[N+]=[N-])CCOCCOCCOCCN=[N+]=[N-])C(C)(C)C)c2ccccc21]

To a solution of Indazole acid 9 (0.05 g, 0.146 mmol, 1 eq) and HOBt (0.026 g, 0.189 mmol, 1.3 eq) in DMF (0.7 mL) was added EDCI. HCl (0.042 g, 0.218 mmol, 1.5 eq) followed by Et₃N (0.074 g, 0.728 mmol, 5 eq) at 0° C. The cooling bath was removed, and the mixture was stirred for 30 mins at rt. The amine NH₂ bis azide (0.104 g, 0.189 mmol, 1.3 eq) was added in DMF (0.1 ml) dropwise, and the mixture was stirred at rt for 3 h. The mixture was directly added onto a C18 column and purified by reverse-phase column chromatography using a gradient 0-50% acetonitrile in water (+0.1% formic acid) to afford the desired product. 1H-NMR and HPLC showed the presence of impurities. The material was re-purified by silica-gel column chromatography using a gradient 0-5% methanol in ethyl acetate to afford the desired product 11 in 29% yield (37 mg). MS ESI+ m/z=876.7 [MH]+.

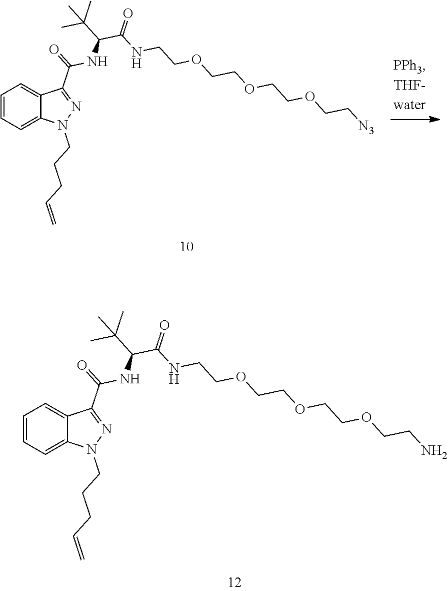 [SMILES: C=CCCCn1nc(C(=O)N[C@H](C(=O)NCCOCCOCCOCCN=[N+]=[N-])C(C)(C)C)c2ccccc21 ; C=CCCCn1nc(C(=O)N[C@H](C(=O)NCCOCCOCCOCCN)C(C)(C)C)c2ccccc21]

To a mixture of Azide 10 (122 mg, 0.22 mmol) dissolved in THE (2 ml)-water (0.2 ml) at rt was added PPh₃ (71 mg, 0.27 mmol, 1.2 eq). The mixture was stirred at rt for 2 h and then more water (0.8 ml) was added, and the mixture was stirred overnight at rt and then concentrated under reduced pressure. The residue obtained was purified by reverse-phase column chromatography (C18) using a gradient 0-50% ACN in water (+0.1% formic acid). The residue obtained was taken up in 0.5M HCl (0.1 ml) and concentrated to dryness. This procedure was repeated twice to afford the desired product 12 as a hydrochloride salt in 74% yield (92 mg). MS ESI+ m/z=518.3 [MH]+.

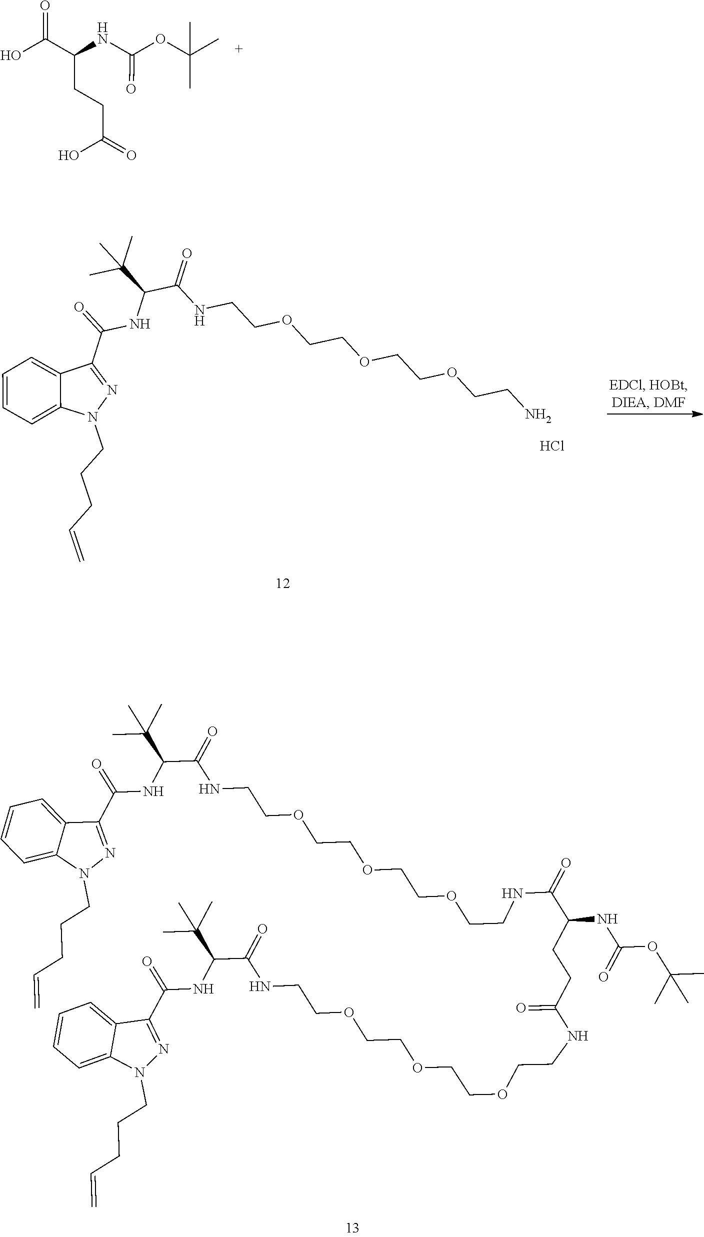 [SMILES: CC(C)(C)OC(=O)N[C@@H](CCC(=O)O)C(=O)O ; C=CCCCn1nc(C(=O)N[C@H](C(=O)NCCOCCOCCOCCN)C(C)(C)C)c2ccccc21 ; Cl ; C=CCCCn1nc(C(=O)N[C@H](C(=O)NCCOCCOCCOCCNC(=O)CC[C@H](NC(=O)OC(C)(C)C)C(=O)NCCOCCOCCOCCNC(=O)[C@@H](NC(=O)c2nn(CCCC=C)c3ccccc23)C(C)(C)C)C(C)(C)C)c2ccccc21]

To a solution of N-Boc-D-Glutamic acid (20 mg, 0.08 mmol, 1 eq) in DMF (0.5 ml) was added HOBt (32 mg, 0.178 mmol, 2.2 eq), EDCI (62 mg, 0.324 mmol, 4 eq), and then DIEA (71 ul, 0.404 mmol, 5 eq) at 0° C. The cooling bath was removed, and the mixture was stirred for 1 h at rt. A solution of the HCl amine 12 (94 mg, 0.17 mmol, 2.1 eq) in DMF (0.25 ml) was added dropwise, followed by DIEA (42 μl, 0.243 mmol, 3 eq), and the mixture was stirred for 20 h at rt. Water was added, and the product was extracted with ethyl acetate three times. The organic phases were combined, washed with aqueous saturated NaHCO₃ and brine, dried, filtered, and then concentrated. The residue obtained was purified by silica-gel column chromatography using a gradient 0-10% methanol in ethyl acetate to afford the desired product 13 in 40% yield (40 mg). MS ESI+ m/z=1246.7 [MH]+.

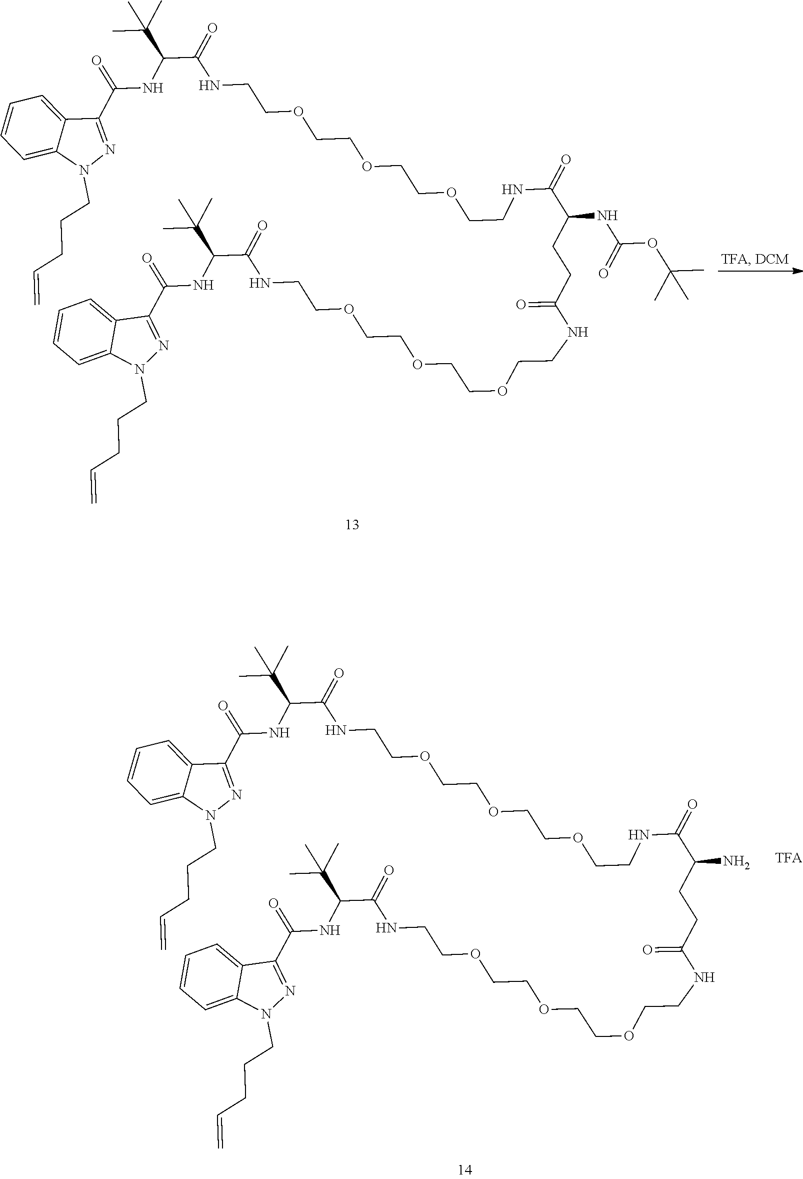 [SMILES: C=CCCCn1nc(C(=O)N[C@H](C(=O)NCCOCCOCCOCCNC(=O)CC[C@H](NC(=O)OC(C)(C)C)C(=O)NCCOCCOCCOCCNC(=O)[C@@H](NC(=O)c2nn(CCCC=C)c3ccccc23)C(C)(C)C)C(C)(C)C)c2ccccc21 ; C=CCCCn1nc(C(=O)N[C@H](C(=O)NCCOCCOCCOCCNC(=O)CC[C@H](N)C(=O)NCCOCCOCCOCCNC(=O)[C@@H](NC(=O)c2nn(CCCC=C)c3ccccc23)C(C)(C)C)C(C)(C)C)c2ccccc21]

To a solution of 13 (40 mg, 0.032 mmol) in anhydrous DCM (0.63 ml) at 0° C. was added TFA (0.3 ml) dropwise. The cooling bath was removed, and the mixture was stirred at rt for 3.5 h and then concentrated under reduced pressure to afford the desired product 14 as a crude mixture (41 mg, quant.), which was used for the following step without further purification. MS ESI+ m/z=1147.2 [MH]+.

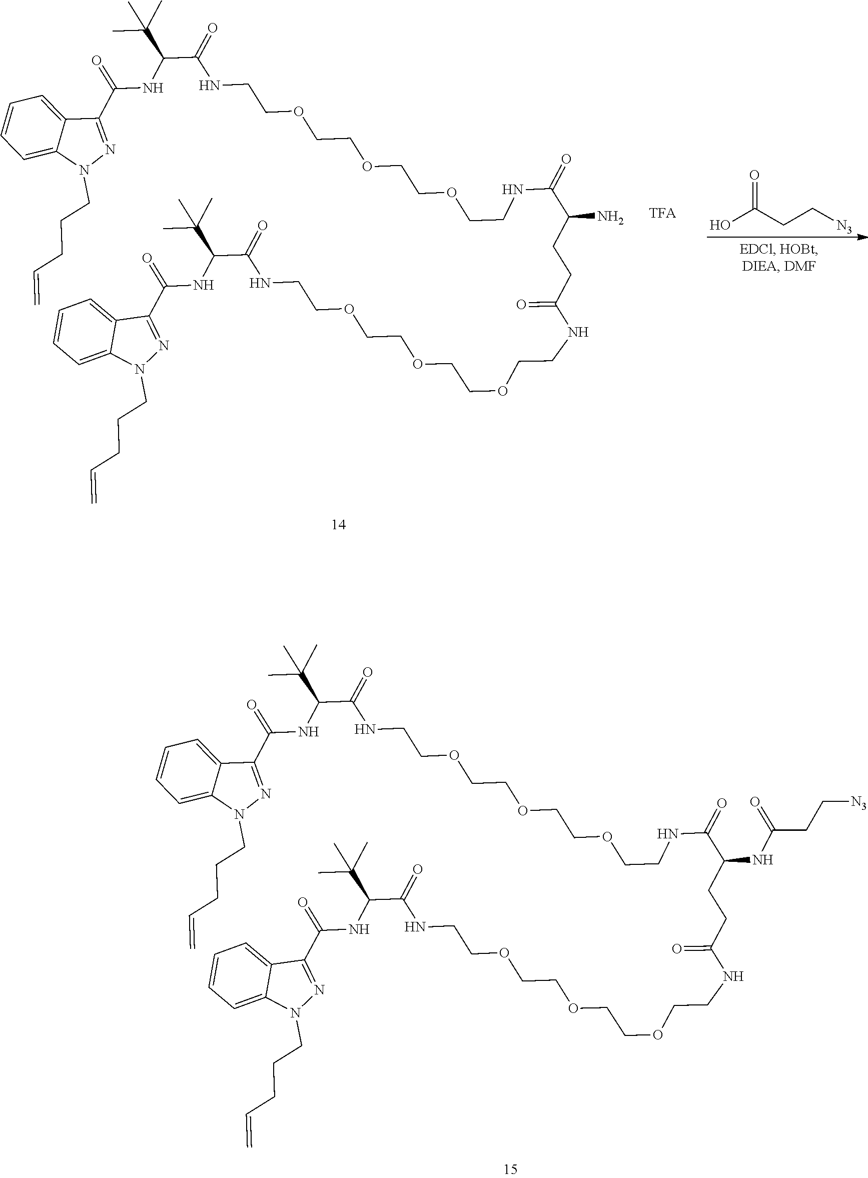 [SMILES: C=CCCCn1nc(C(=O)N[C@H](C(=O)NCCOCCOCCOCCNC(=O)CC[C@H](N)C(=O)NCCOCCOCCOCCNC(=O)[C@@H](NC(=O)c2nn(CCCC=C)c3ccccc23)C(C)(C)C)C(C)(C)C)c2ccccc21 ; [N-]=[N+]=NCCC(=O)O ; C=CCCCn1nc(C(=O)N[C@H](C(=O)NCCOCCOCCOCCNC(=O)CC[C@H](NC(=O)CCN=[N+]=[N-])C(=O)NCCOCCOCCOCCNC(=O)[C@@H](NC(=O)c2nn(CCCC=C)c3ccccc23)C(C)(C)C)C(C)(C)C)c2ccccc21]

To a mixture of Azido propionic acid (5 mg, 0.048 mmol, 1.5 eq) in DCM (0.1 ml) was added HOBt (6 mg, 0.042 mmol, 1.3 eq), followed by EDCI (10 mg, 0.064 mmol, 2 eq) and DIEA (17 μl, 0.097 mmol, 3 eq), and the mixture was stirred at rt for 30 mins. A mixture composed of TFA salt 14 (40 mg, 0.032 mmol, 1 eq) and DIEA (7 ul, 0.039 mmol, 1.2 eq) in DCM (0.07 ml) was added dropwise to the activated acid. The mixture was stirred for 10 h at rt and then concentrated. The residue obtained was purified by silica-gel column chromatography using a gradient 0-10% methanol in ethyl acetate to afford the desired product 15 in 83% yield (33 mg). MS ESI+ m/z=1265.1 [MNa]+.

### Example 2: BA-116 Conjugate

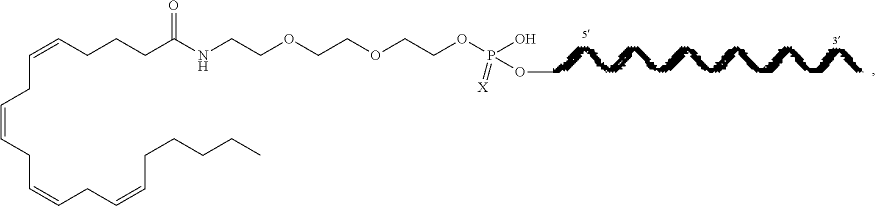

wherein X is O or S.

### BA-116: 2-cyanoethyl (2-(2-(2-((5Z,8Z,11Z,14Z)-icosa-5,8,11,14-tetraenamido)ethoxy) ethoxy)ethyl)diisopropylphosphoramidite

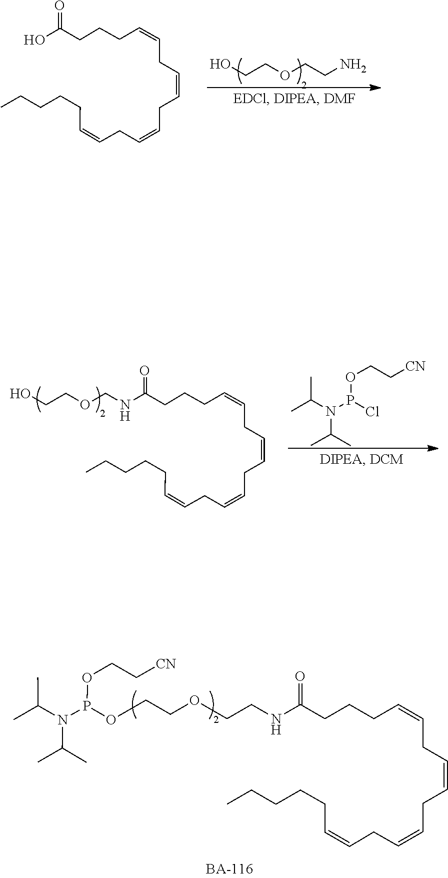

### Step 1: (5Z,8Z,11Z,14Z)—N-(2-(2-(2-hydroxyethoxy)ethoxy)ethyl)icosa-5,8,11,14-tetraenamide

To a solution of arachidonic acid (1.0 g, 3.3 mmol) in DCM (20 mL) at room temperature was added EDCI (586 mg, 3.61 mmol) and the reaction mixture was stirred at room temperature for 1.5 hours. 2-(2-(2-aminoethoxy)ethoxy)ethan-1-ol (0.9 mL, 6.57 mmol) was added dropwisely and the reaction mixture was stirred for room temperature for 2 hours. The mixture was diluted with DCM (50 mL), washed with water (3×20 mL) and bine (20 mL). The organic layer was dried with Na₂SO₄, filtered and concentrated. The residue was purified by silica gel column chromatography using a gradient 0 to 10% MeOH in ethyl acetate to afford the titled compound (867 mg, 61% yield) as a yellow oil. MS (ESI): m/z=436.3 [M+H]⁺, 458.1 [M+Na]⁺.

### Step 2: 2-cyanoethyl (2-(2-(2-((5Z,8Z,11Z,14Z)-icosa-5,8,11,14-tetraenamido)ethoxy) ethoxy)ethyl)diisopropylphosphoramidite

To a stirred solution of (5Z,8Z,11Z,14Z)—N-(2-(2-(2-hydroxyethoxy)ethoxy)ethyl)icosa-5,8,11,14-tetraenamide (680 mg, 1.56 mmol) in DCM (36 mL) at room temperature was added DIEA (1.76 mL, 10.1 mmol) followed by 3-((chloro(diisopropylamino)phosphaneyl)oxy)propanenitrile (0.38 mL, 1.72 mmol) dropwise, and the reaction was stirred at room temperature for 1.5 hours. The reaction mixture was quenched with aqueous saturated NaHCO₃ (150 mL). The product was extracted with DCM (2×300 mL), washed with aqueous saturated NaHCO₃ (150 mL), brine (300 mL), dried with Na₂SO₄, filtered and concentrated. The residue was purified by silica gel column chromatography eluted with 0 to 80% ethyl acetate in hexane to afford the titled compound (632 mg, 64% yield) as a yellow oil. MS (ESI): m/z=636.3 [M+H]⁺, 658.3 [M+Na]⁺.

BA-116 will be conjugated to an oligo sense strand. For example, a solution of BA-116 will be combined with a solution of BTT activator and coupled, sulfurized with a solution of DDTT solution and deprotected with NH₄OH solution at an elevated temperature.

### Example 3: BA-126 Conjugates

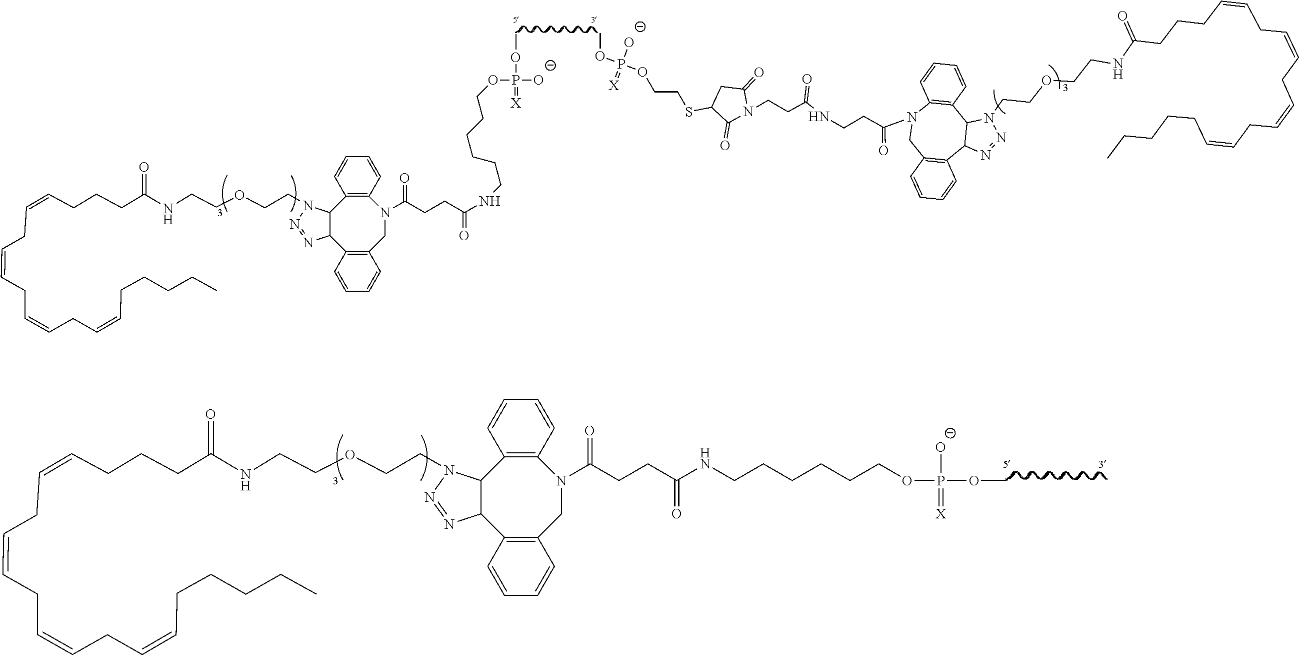

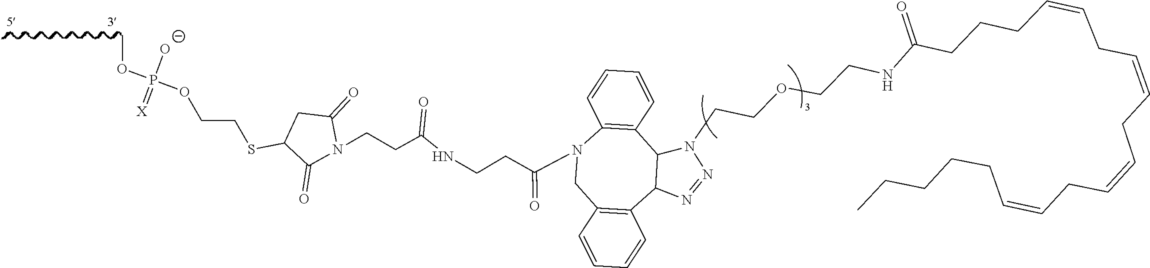

wherein X is O or S.

### BA-126: (5Z,8Z,11Z,14Z)—N-(2-(2-(2-(2-azidoethoxy)ethoxy)ethoxy)ethyl)icosa-5,8,11,14-tetraenamide

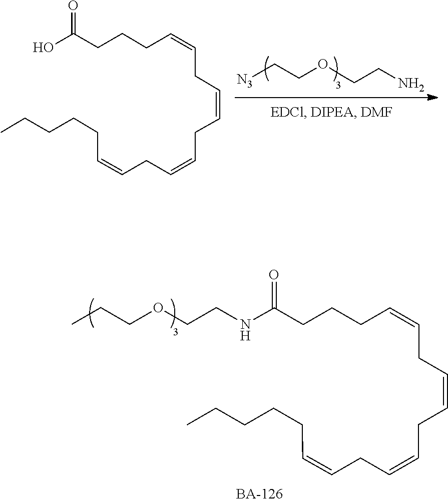

To a solution of arachidonic acid (125 mg, 0.41 mmol) in DMF (2.5 mL) at 0° C., were successively added HOBt (61 mg, 0.45 mmol) and EDCI (102 mg, 0.53 mmol). The mixture was stirred for 45 minutes at room temperature followed by 2-(2-(2-(2-azidoethoxy)ethoxy)ethoxy)ethan-1-amine (116 mg, 0.534 mmol). The mixture was stirred for another 3 hours at room temperature. The mixture was diluted with EtOAc (50 mL) and washed with brine (20 mL×3). The organic phase was dried with Na₂SO₄, filtered and concentrated. The residue was purified by silica-gel column chromatography using a gradient 0-5% methanol in EtOAc to afford the titled compound (119 mg, 57% yield) as a colorless oil. MS (ESI): m/z=505.3 [M+H]⁺.

BA-126 will be conjugated to an oligo sense strand according to general procedure type I, IIA/B, and/or III.

### Example 4: BA-139 Conjugate

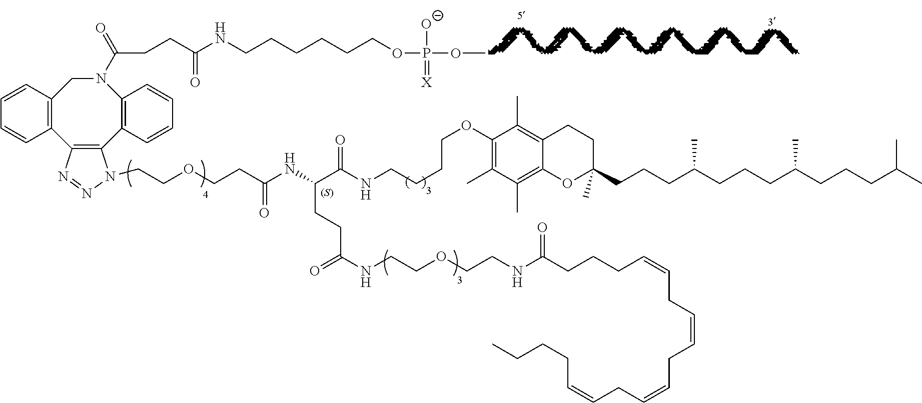

wherein X is O or S.

### BA-139 (S)-2-(1-azido-3,6,9,12-tetraoxapentadecan-15-amido)-N5-((17Z,20Z,23Z,26Z)-13-oxo-3,6,9-trioxa-12-azadotriaconta-17,20,23,26-tetraen-1-yl)-N1-(6-(((R)-2,5,7,8-tetramethyl-2-((4R,8R)-4,8,12-trimethyltridecyl)chroman-6-yl)oxy)hexyl)pentanediamide

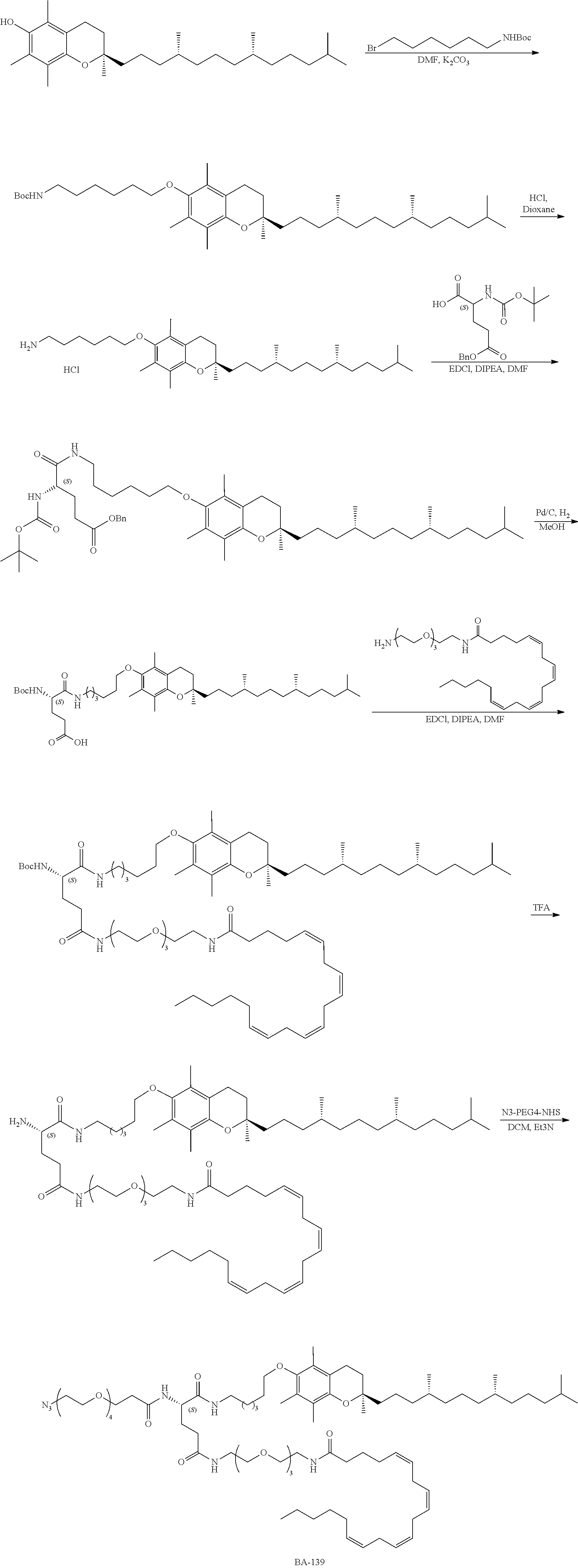

### Step 1: tert-butyl (6-(((R)-2,5,7,8-tetramethyl-2-((4R,8R)-4,8,12-trimethyltridecyl)chroman-6-yl)oxy)hexyl)carbamate

To a stirred solution of Tocopherol (2.0 g, 4.6 mmol) in dry DMF (12 mL) was added the tert-butyl (6-bromohexyl)carbamate (2.34 g, 8.36 mmol) followed by K₂CO₃ (1.92 g, 13.9 mmol) at room temperature under inert atmosphere. The resulting reaction mixture was gradually heated up to 70° C. and stirred at this temperature for 16 hours. Another portion of tert-butyl (6-bromohexyl)carbamate (1 mL) was added and the mixture was stirred at 65° C. for 2 hours. The reaction mixture was diluted with ice-cold water (50 mL) and extracted with EtOAc (3×75 mL). The combined organic extracts were washed with water (75 mL), brine (50 mL), dried over anhydrous Na₂SO₄ and concentrated under reduced pressure. The crude material was purified by silica gel column chromatography using a gradient 0-5% ethyl acetate in hexanes to afford the titled compound (1.67 g, 57% yield) as an oil. MS (ESI): m/z=629.7 [M+H]⁺.

### Step 2: 6-(((R)-2,5,7,8-tetramethyl-2-((4R,8R)-4,8,12-trimethyltridecyl)chroman-6-yl)oxy)hexan-1-amine hydrochloride

tert-butyl (6-(((R)-2,5,7,8-tetramethyl-2-((4R,8R)-4,8,12-trimethyltridecyl)chroman-6-yl)oxy)hexyl)carbamate (1.62 g, 2.57 mmol) was dissolved 4 N HCl in dioxane (16.3 mL) at 0° C. then stirred at rt for 5 h. The dioxane was removed and the resulting material was dried in vacuo for 8 h to afford the titled compound (1.46 g, 100% yield). MS (ESI): m/z=530.3 [M+H]+.

### Step 3: benzyl (S)-4-((tert-butoxycarbonyl)amino)-5-oxo-5-((6-(((R)-2,5,7,8-tetramethyl-2-((4R,8R)-4,8,12-trimethyltridecyl)chroman-6-yl)oxy)hexyl)amino)pentanoate

To a solution of (S)-5-(benzyloxy)-2-((tert-butoxycarbonyl)amino)-5-oxopentanoic acid (200 mg, 0.593 mmol), 6-(((R)-2,5,7,8-tetramethyl-2-((4R,8R)-4,8,12-trimethyltridecyl)chroman-6-yl)oxy)hexan-1-amine hydrochloride (336 mg, 0.593 mmol), Et₃N (0.12 ml, 0.77 mmol,) and HOBt (88 mg, 0.65 mmol) in DCM (7 mL) at 0° C. was added EDCI (147 mg, 0.771 mmol). After 15 minutes, the cooling bath was removed and the mixture was stirred for 18 hours at room temperature, then diluted with water and extracted with DCM. The combined organic phases were washed with brine, dried with Na₂SO₄, filtered then concentrated. The residue was purified by silica-gel column chromatography using a gradient 0-100% ethyl acetate in hexanes to afford the titled compound (342 mg, 68% yield) as a yellow oil. MS (ESI): m/z=871.3 [M+Na]+.

### Step 4: (S)-4-((tert-butoxycarbonyl)amino)-5-oxo-5-((6-(((R)-2,5,7,8-tetramethyl-2-((4R,8R)-4,8,12-trimethyltridecyl)chroman-6-yl)oxy)hexyl)amino)pentanoic acid

A solution of benzyl (S)-4-((tert-butoxycarbonyl)amino)-5-oxo-5-((6-(((R)-2,5,7,8-tetramethyl-2-((4R,8R)-4,8,12-trimethyltridecyl)chroman-6-yl)oxy)hexyl)amino)pentanoate (100 mg, 0.118 mmol) in methanol (2.0 mL) was stirred under 1 atm of H₂ (g) in the presence of Pd/C (125 mg, 0.187 mmol) for 5 hours. The catalyst was filtered off and the filtrate was concentrated to afford the crude as the titled compound (87 mg, 97% yield). MS ESI+781.2 [M+Na]+.

### Step 5: tert-butyl ((S,30Z,33Z,36Z,39Z)-8,12,26-trioxo-1-(((R)-2,5,7,8-tetramethyl-2-((4R,8R)-4,8,12-trimethyltridecyl)chroman-6-yl)oxy)-16,19,22-trioxa-7,13,25-triazapentatetraconta-30,33,36,39-tetraen-9-yl)carbamate

To a solution of (S)-4-((tert-butoxycarbonyl)amino)-5-oxo-5-((6-(((R)-2,5,7,8-tetramethyl-2-((4R,8R)-4,8,12-trimethyltridecyl)chroman-6-yl)oxy)hexyl)amino)pentanoic acid (0.172 g, 0.227 mmol), (5Z,8Z,11Z,14Z)—N-(2-(2-(2-(2-aminoethoxy)ethoxy)ethoxy)ethyl)icosa-5,8,11,14-tetraenamide (0.11 g, 0.23 mmol), Et₃N (0.04 mL, 0.30 mmol) and HOBt (0.045 g, 0.25 mmol) in DCM (3 mL) at 0° C. was added EDCI (0.056 g, 0.30 mmol). After 30 minutes, the cooling bath was removed and the mixture was stirred for 14 hours at room temperature, then concentrated. The residue was purified by silica-gel column chromatography using a gradient 0-4% methanol in ethyl acetate to afford the titled compound (158 mg, 57% yield) as a yellow oil. MS (ESI): m/z=1241.3 [M+Na]⁺.

### Step 6: (S)-2-amino-N5-((17Z,20Z,23Z,26Z)-13-oxo-3,6,9-trioxa-12-azadotriaconta-17,20,23,26-tetraen-1-yl)-N1-(6-(((R)-2,5,7,8-tetramethyl-2-((4R,8R)-4,8,12-trimethyltridecyl)chroman-6-yl)oxy)hexyl)pentanediamide

TFA (0.35 mL) was added dropwise at 0° C. to a solution of Boc protected bis amide, tert-butyl ((S,30Z,33Z,36Z,39Z)-8,12,26-trioxo-1-(((R)-2,5,7,8-tetramethyl-2-((4R,8R)-4,8,12-trimethyltridecyl)chroman-6-yl)oxy)-16,19,22-trioxa-7,13,25-triazapentatetraconta-30,33,36,39-tetraen-9-yl)carbamate (0.133 g, 0.109 mmol) in DCM (0.7 mL). The mixture was stirred for 2 hours at room temperature and was concentrated. The residue obtained was partitioned between DCM (20 mL) and aq. sat. NaHCO₃ (5 mL). The aqueous phase was extracted with DCM (2×20 mL). The combined organic phase was washed with NaHCO₃, brine, dried with Na₂SO₄, filtered then concentrated to afford the titled compound (121 mg, 99% yield). MS (ESI): m/z=1119.5 [M+H]⁺.

### Step 7: (S)-2-(1-azido-3,6,9,12-tetraoxapentadecan-15-amido)-N5-((17Z,20Z,23Z,26Z)-13-oxo-3,6,9-trioxa-12-azadotriaconta-17,20,23,26-tetraen-1-yl)-N1-(6-(((R)-2,5,7,8-tetramethyl-2-((4R,8R)-4,8,12-trimethyltridecyl)chroman-6-yl)oxy)hexyl)pentanediamide

NHS ester, 2,5-dioxopyrrolidin-1-yl 1-azido-3,6,9,12-tetraoxapentadecan-15-oate (0.05 g, 0.13 mmol) was added in DCM (0.1 mL) at 0° C. to a solution of (S)-2-amino-N5-((17Z,20Z,23Z,26Z)-13-oxo-3,6,9-trioxa-12-azadotriaconta-17,20,23,26-tetraen-1-yl)-N1-(6-(((R)-2,5,7,8-tetramethyl-2-((4R,8R)-4,8,12-trimethyltridecyl)chroman-6-yl)oxy)hexyl)pentanediamide (0.121 g, 0.1 mmol) and Et₃N (0.02 g, 0.199 mmol) in DCM (0.95 mL). The mixture was stirred for 14 hours at room temperature. The reaction mixture was concentrated, and the residue was purified by silica-gel column using a gradient 50 to 100% ethyl acetate in hexanes followed by a gradient 0 to 20% methanol in ethyl acetate to afford the titled compound (37 mg, 27% yield) as a yellow oil. MS (ESI): m/z=1414.3 [M+Na]⁺.

BA-139 will be conjugated to an oligo sense strand according to general procedure type I, IIA/B, and/or III.

### Example 5: BA-119 Conjugate

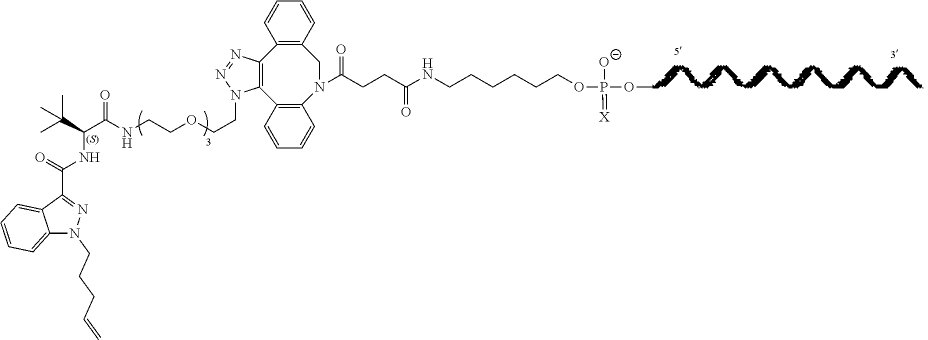

wherein X is O or S.

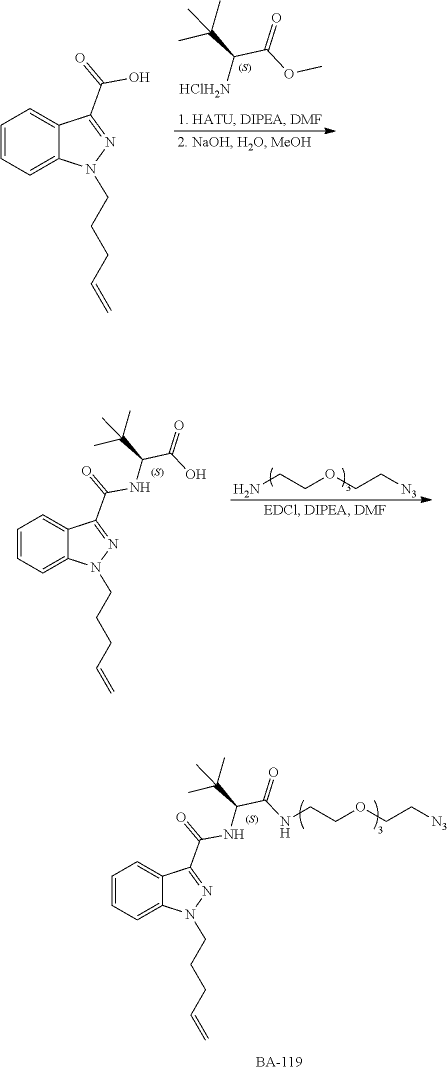

### Step 1: methyl (S)-3,3-dimethyl-2-(1-(pent-4-en-1-yl)-1H-indazole-3-carboxamido)butanoate

To a solution of 1-(pent-4-en-1-yl)indazole-3-carboxylic acid (2.46 g, 10.7 mmol) and methyl (2S)-methyl (S)-2-amino-3,3-dimethylbutanoate hydrogen chloride (1.94 g, 10.7 mmol) in DMF (50 mL) was added HATU (6.09 g, 16.0 mmol) and DIEA (5.5 g, 42 mmol), the mixture was stirred for 2 hours at room temperature under argon atmosphere. The reaction was quenched with water at room temperature. The resulting mixture was extracted with EtOAc. The combined organic layers were washed with brine, dried over anhydrous Na₂SO₄, filtered and concentrated under reduced pressure to afford the crude product (4.8 g 100% yield) as the titled compound was used in the next step directly without further purification. 1H NMR (300 MHz, DMSO-d₆) δ 8.15 (dt, J=8.2, 1.0 Hz, 1H), 7.80 (dt, J=8.6, 0.9 Hz, 1H), 7.66 (d, J=9.3 Hz, 1H), 7.58-7.43 (m, 1H), 7.31 (ddd, J=7.9, 6.9, 0.8 Hz, 1H), 5.84 (ddt, J=16.7, 10.2, 6.2 Hz, 1H), 5.10-4.98 (m, 1H), 5.03-4.94 (m, 1H), 4.59-4.40 (m, 3H), 3.72 (s, 3H), 2.70 (s, 2H), 2.13-1.88 (m, 5H), 1.32-1.13 (m, 1H), 1.03 (s, 9H). MS (ESI): m/z=357.2.

### Step 2: (S)-3,3-dimethyl-2-(1-(pent-4-en-1-yl)-1H-indazole-3-carboxamido)butanoic acid

A mixture of methyl (S)-3,3-dimethyl-2-(1-(pent-4-en-1-yl)-1H-indazole-3-carboxamido) butanoate (4.4 g, 12 mmol) and NaOH (1.98 g, 49.572 mmol, 1 N in water) in MeOH (45 mL) was stirred for overnight at room temperature under argon atmosphere. The mixture was acidified to pH 5 with acetic acid (5.0 eq). The reaction was quenched with water at room temperature. The resulting mixture was extracted with EtOAc, dried over anhydrous Na₂SO₄. After filtration, the filtrate was concentrated under reduced pressure, to afford the crude product as the titled compound (3.7 g, 87% yield) as an orange oil, was used in the next step without further purification. 1H NMR (300 MHz, DMSO-d₆) δ 12.91 (s, 1H), 8.15 (dt, J=8.2, 1.1 Hz, 1H), 7.84-7.69 (m, 1H), 7.56 (d, J=9.4 Hz, 1H), 7.48 (ddd, J=8.4, 6.9, 1.2 Hz, 1H), 7.30 (ddd, J=8.0, 6.9, 0.8 Hz, 1H), 5.83 (ddt, J=16.8, 10.3, 6.2 Hz, 1H), 5.09-4.93 (m, 2H), 4.53 (t, J=6.7 Hz, 2H), 4.46-4.38 (m, 1H), 2.69 (s, OH), 2.12-1.88 (m, 4H), 1.03 (s, 8H). MS (ESI): m/z=343.2 [M+H]⁺.

### Step 3: (S)—N-(1-azido-15,15-dimethyl-13-oxo-3,6,9-trioxa-12-azahexadecan-14-yl)-1-(pent-4-en-1-yl)-1H-indazole-3-carboxamide

To a solution of (S)-3,3-dimethyl-2-(1-(pent-4-en-1-yl)-1H-indazole-3-carboxamido)butanoic acid (24 mg, 0.07 mmol) and HOBt (26 mg, 0.19 mmol) in DMF (0.7 mL), was added EDCI (42 mg, 0.22 mmol) followed by Et₃N (0.1 ml, 0.7 mmol) at 0° C. The cooling bath was removed and the mixture was stirred for 30 minutes at room temperature. The 2-(2-(2-(2-azidoethoxy)ethoxy) ethoxy)ethan-1-amine (48 mg, 0.22 mmol) was added in DMF (0.1 ml) dropwise and the mixture was stirred at room temperature for 1 hour. The mixture was directly added onto a C18 column and purified by reverse-phase column chromatography using a gradient 0-50% acetonitrile in water (+0.1% formic acid) to afford the titled compound (18 mg, 23% yield). The reaction was repeated (acid: 150 mg scale) to afford the titled compound in (122 mg, 51% yield) as a clear oil. MS (ESI): m/z=544.5 [M+H]⁺.

BA-119 was conjugated to an oligo sense strand according to general procedure type I. The product (MW: 8255.08 g/mol) was made with 98% purity and confirmed with HPLC and LCMS (m/z: 8253.29).

### Example 6: BA-155 Conjugate

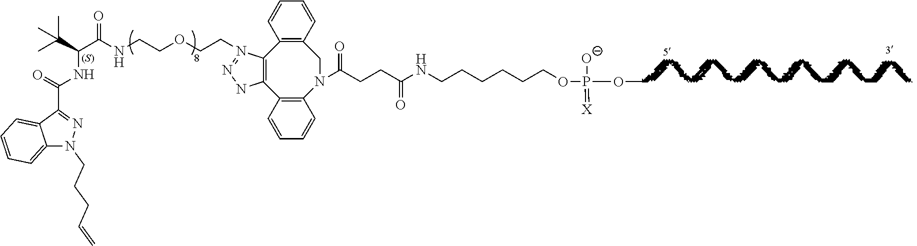

wherein X is O or S.

### BA-155: (S)—N-(1-azido-30,30-dimethyl-28-oxo-3,6,9,12,15,18,21,24-octaoxa-27-azahentriacontan-29-yl)-1-(pent-4-en-1-yl)-1H-indazole-3-carboxamide

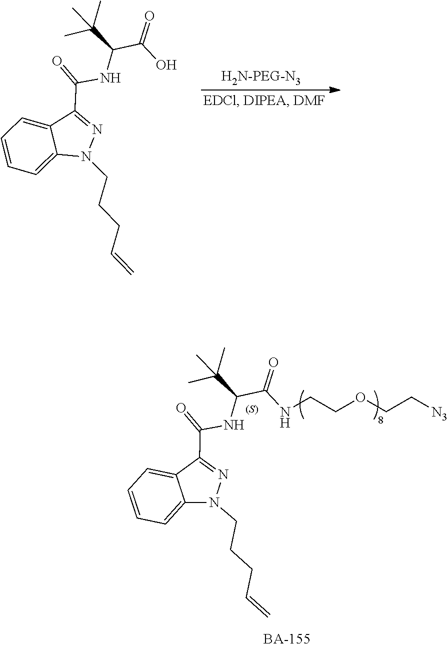

Used the same procedure as in the preparation of BA-119 by replace the amine with 26-azido-3,6,9,12,15,18,21,24-octaoxahexacosan-1-amine to afford the titled compound (140 mg, 63% yield) as a clear oil. MS (ESI): m/z=786.0 [M+Na]⁺.

BA-155 was conjugated to an oligo sense strand according to general procedure type T. The product (MW: 8064.00 g/mol) was made with 96% purity and confirmed by HPLC and LCMS (m/z: 8062.65).

### Example 7: BA-138 Conjugate

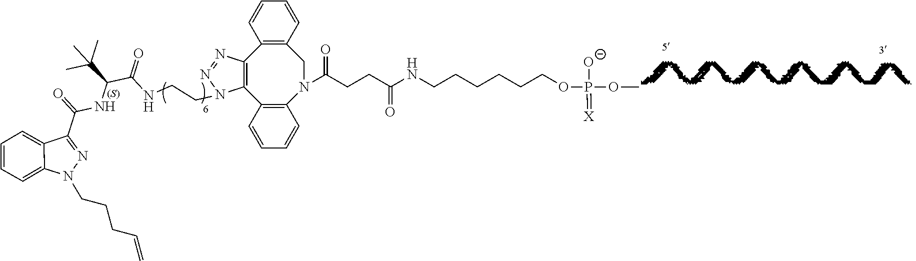

wherein X is O or S.

BA-138: (S)—N-(1-((12-azidododecyl)amino)-3,3-dimethyl-1-oxobutan-2-yl)-1-(pent-4-en-1-yl)-1H-indazole-3-carboxamide

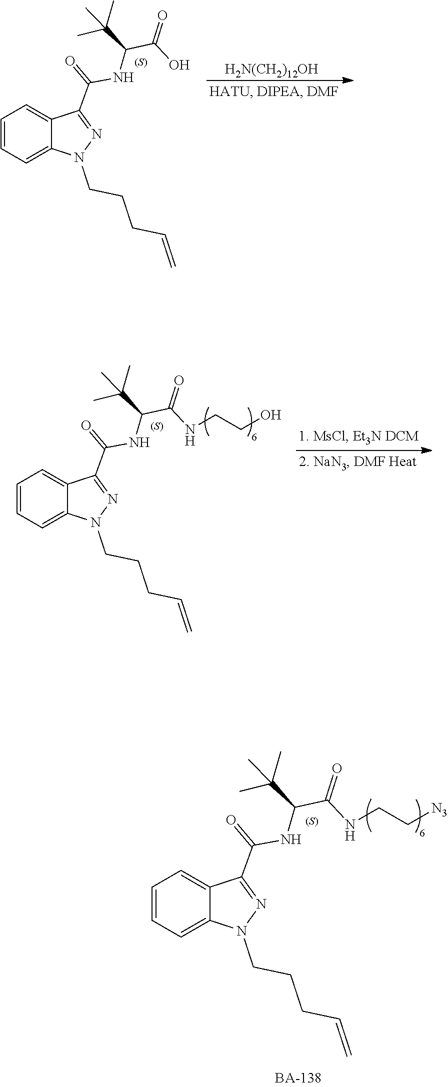

### Step 1: (S)—N-(1-((12-hydroxydodecyl)amino)-3,3-dimethyl-1-oxobutan-2-yl)-1-(pent-4-en-1-yl)-1H-indazole-3-carboxamide

HATU (2.29 g, 6.0 mmol) and DIPEA (3.5 mL, 20 mmol) were added to a solution of (S)-3,3-dimethyl-2-(1-(pent-4-en-1-yl)-1H-indazole-3-carboxamido)butanoic acid (1.38 g, 4.0 mmol) and aminododecanol (1.1 mL, 6.0 mmol) in anhydrous DMF (12 mL) at 0° C. The reaction mixture was then allowed to stir at room temperature for overnight. The reaction mixture was poured into ethyl acetate-hexanes (9:1, 200 mL) and 50% saturated NH₄Cl (120 mL). The organic layer was washed with water (2×120 mL) and with brine (120 mL). The organic layer was dried over anhydrous sodium sulfate, filtered, and concentrated under reduced pressure. The residue was purified by FCC on silica gel (120 g silicycle, 0 to 50% ethyl acetate/hexanes) to afford the titled compound (1.52 g, 72%) as a colorless oil. MS (ESI): m/z=527 [M+H]⁺.

### Step 2: (S)-12-(3,3-dimethyl-2-(1-(pent-4-en-1-yl)-1H-indazole-3-carboxamido)butanamido) dodecyl methanesulfonate

MsCl (0.34 mL, 4.3 mmol) was added dropwise to a solution of (S)—N-(1-((12-hydroxydodecyl)amino)-3,3-dimethyl-1-oxobutan-2-yl)-1-(pent-4-en-1-yl)-1H-indazole-3-carboxamide (1.52 g, 2.89 mmol) and triethylamine (1.2 mL, 8.7 mmol) in anhydrous DCM (30 mL) at −40° C. The reaction mixture was then allowed to warm to 0° C. After 70 min, the reaction mixture was diluted with DCM (20 mL) and washed with saturated NaHCO₃ (50 mL). The aqueous layer was extracted with DCM (2×25 mL). The combined organic layers were dried over anhydrous sodium sulfate, filtered, and concentrated under reduced pressure. The resulting residue was diluted with ethyl acetate and then concentrated under reduced pressure to afford the titled compound (1.78 g, 100% yield) as pale yellow oil was used without further purification MS (ESI): m/z=M+H=605.6 [M+H]⁺, 627.4 [M+Na]⁺).

### Step 3: (S)—N-(1-((12-azidododecyl)amino)-3,3-dimethyl-1-oxobutan-2-yl)-1-(pent-4-en-1-yl)-1H-indazole-3-carboxamide

A suspension of (S)-12-(3,3-dimethyl-2-(1-(pent-4-en-1-yl)-1H-indazole-3-carboxamido)butanamido) dodecyl methanesulfonate (1.75 g, 2.89 mmol) and sodium azide (0.47 g, 7.23 mmol) in anhydrous DMF (12 mL) were heated to 80° C. After 16 h, the reaction mixture was cooled to room temperature, poured into ethyl acetate-hexanes (7:1, 100 mL, v/v) and washed with 50% saturated NaHCO₃ (100 mL). The aqueous layer was extracted with ethyl acetate-hexane (7:1, 100 mL). The combined organic layers were washed with water (2×100 mL) and with brine (100 mL). The organic layer was dried over anhydrous sodium sulfate, filtered, and concentrated under reduced pressure. The resulting crude residue was purified by FCC on silica gel (40 g silicycle, 0 to 25% ethyl acetate/hexanes) to afford the title compound (1.41 g, 88%) as a clear and colorless oil. MS (ESI): m/z=552.5 [M+H]⁺, 574.1 [M+Na]⁺.

BA-138 was conjugated to an oligo sense strand according to general procedure type I. The product (MW: 7955.94 g/mol) was made with 99% purity and confirmed by HPLC and LCMS (m/z: 7954.29).

### Example 8: BA-131 Conjugate

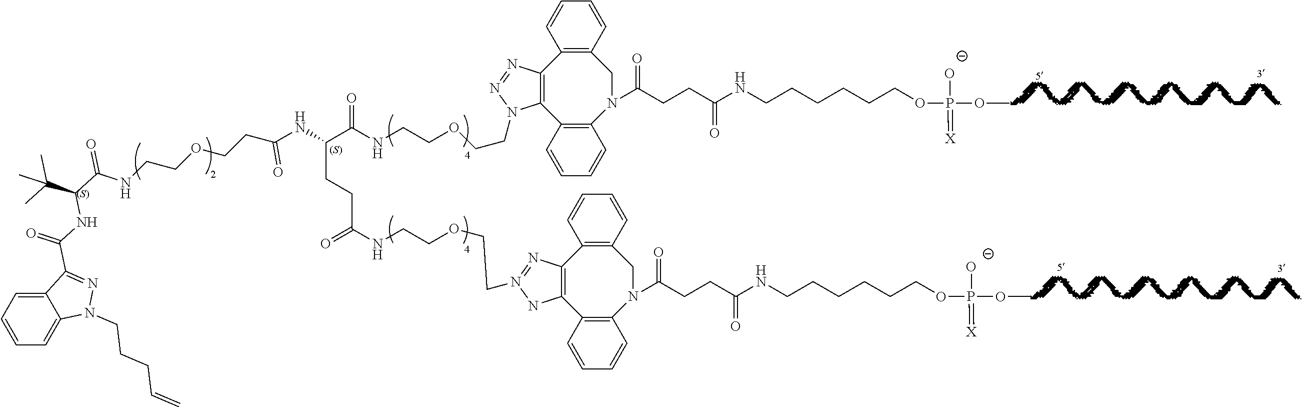

wherein X is O or S.

BA-131 (S)—N1,N5-bis(14-azido-3,6,9,12-tetraoxatetradecyl)-2-((S)-3-(tert-butyl)-1,4-dioxo-1l-(1-(pent-4-en-1-yl)-1H-indazol-3-yl)-8,11-dioxa-2,5-diazatetradecan-14-amido)pentanediamide

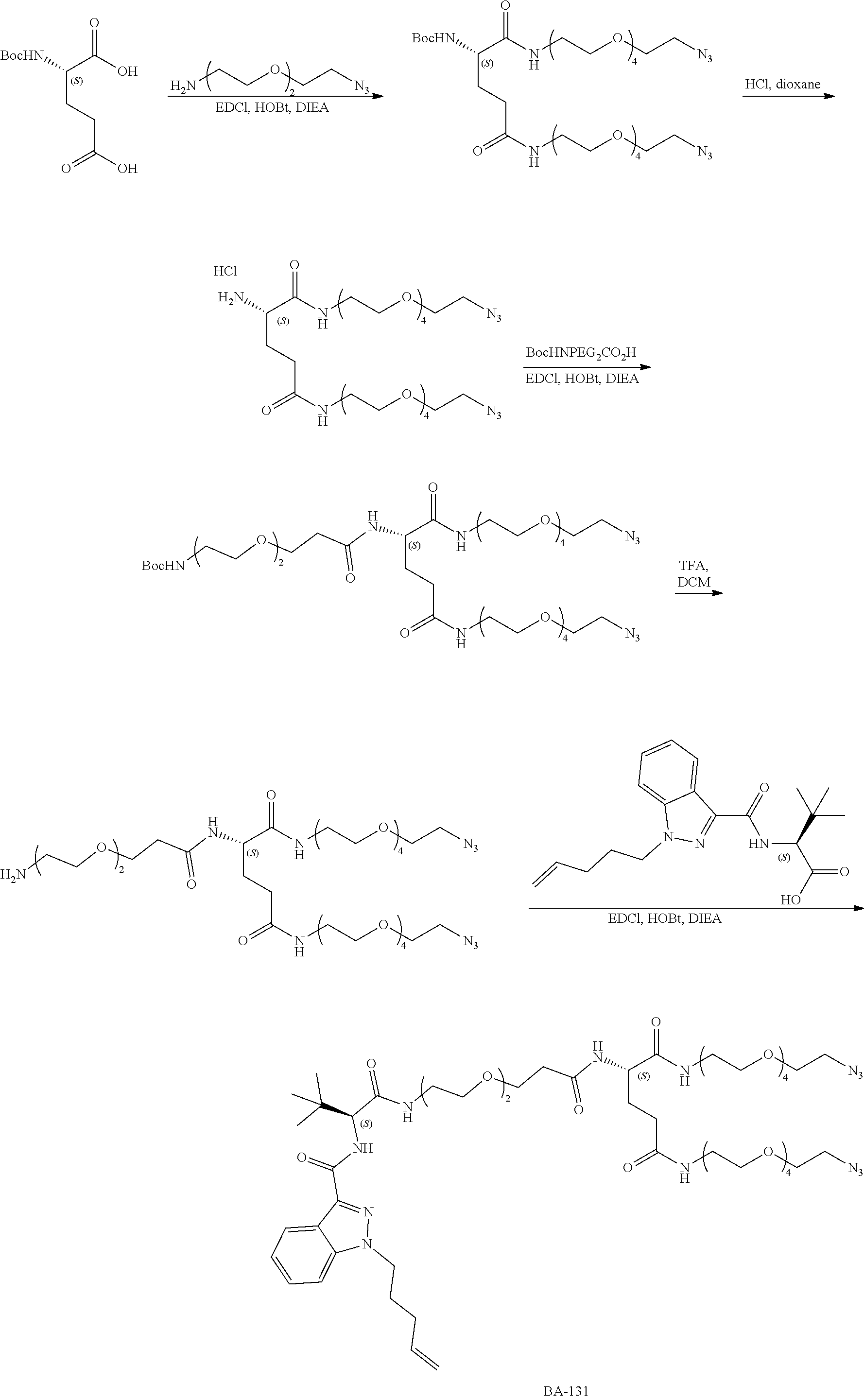

### Step 1: tert-butyl (S)-(1,35-diazido-16,20-dioxo-3,6,9,12,24,27,30,33-octaoxa-15,21-diazapentatriacontan-17-yl)carbamate

To a solution of Boc-Glu-OH (450 mg, 1.82 mmol) in DMF (11 mL) at 0° C. was added HOBt (615 mg, 4.55 mmol) followed by EDCI (869 mg, 4.55 mmol). The mixture was stirred for 30 mins at rt then a solution of 14-azido-3,6,9,12-tetraoxatetradecan-1-amine (1.0 g, 3.8 mmol) and DIEA (0.7 ml, 4.0 mmol) in DCM (5.6 mL) was added dropwise. The mixture was stirred for 16 hours at room temperature then concentrated and purified by C18 reverse-phase column chromatography using a gradient 0-100% acetonitrile in water (+0.1% formic acid) to afford the titled compound (878 mg, 66% yield) as a yellow oil. MS (ESI): m/z=736.1 [M+H]⁺.

### Step 2: (S)-2-amino-N1,N5-bis(14-azido-3,6,9,12-tetraoxatetradecyl)pentanediamide

Combined tert-butyl (S)-(1,35-diazido-16,20-dioxo-3,6,9,12,24,27,30,33-octaoxa-15,21-diazapentatriacontan-17-yl)carbamate (659 mg, 0.896 mmol) and 4 N HCl in dioxane (5.7 mL), stirred the reaction mixture for 4 hours. The solvent was evaporated and the residue was purified by reverse-phase C18 column chromatography using a gradient 0-100% acetonitrile in water (+0.1% formic acid) to afford the titled compound (448 mg, 74% yield). MS (ESI): m/z=636.5 [M+H]⁺.

### Step 3: tert-butyl (S)-(1-azido-19-((14-azido-3,6,9,12-tetraoxatetradecyl)carbamoyl)-16,21-dioxo-3,6,9,12,24,27-hexaoxa-15,20-diazanonacosan-29-yl)carbamate

To a solution of 2,2-dimethyl-4-oxo-3,8,11-trioxa-5-azatetradecan-14-oic acid (0.25 g, 0.901 mmo) and HOBt (0.213 g, 1.578 mmol) in DMF (2 mL), was added EDCI (0.301 g, 1.578 mmol) at 0° C. The cooling bath was removed, and the mixture was stirred for 30 minutes at room temperature. A mixture of (S)-2-amino-N1,N5-bis(14-azido-3,6,9,12-tetraoxatetradecyl) pentanediamide (0.606 g, 0.901 mmol), Et₃N (0.38 mL, 2.704 mmol) in DMF (3.5 mL) was added dropwise and the mixture was stirred at rt overnight. The insoluble material was filtered off, the mixture was concentrated then purified by silica-gel column chromatography using a gradient 0-15% methanol in ethyl acetate to afford the titled compound (287 mg, 36% yield). MS (ESI): m/z=895.1 [M+H]⁺.

### Step 4: (S)-2-(3-(2-(2-aminoethoxy)ethoxy)propanamido)-N1,N5-bis(14-azido-3,6,9,12-tetraoxatetradecyl)pentanediamide

TFA (1.5 ml) was added dropwise at 0° C. to a solution of tert-butyl (S)-(1-azido-19-((14-azido-3,6,9,12-tetraoxatetradecyl)carbamoyl)-16,21-dioxo-3,6,9,12,24,27-hexaoxa-15,20-diazanonacosan-29-yl)carbamate (0.207 g, 0.231 mmol) in DCM (1.5 mL). The cooling bath was removed, and the mixture was stirred for 3 hours at room temperature, then concentrated. The residue was purified by silica-gel column chromatography using a gradient 0-15% methanol in DCM to afford the titled compound as a yellow oil (118 mg, 57% yield). MS (ESI): m/z=794.8 [M+H]⁺.

### Step 5: (S)—N1,N5-bis(14-azido-3,6,9,12-tetraoxatetradecyl)-2-((S)-3-(tert-butyl)-1,4-dioxo-1-(1-(pent-4-en-1-yl)-1H-indazol-3-yl)-8,11-dioxa-2,5-diazatetradecan-14-amido)pentanediamide

To a solution of (S)-3,3-dimethyl-2-(1-(pent-4-en-1-yl)-1H-indazole-3-carboxamido)butanoic acid (0.045 g, 0.131 mmol) and HOBt (0.018 g, 0.131 mmol) in DMF (0.8 mL), was added EDCI (0.075 g, 0.393 mmol) followed by DTEA (0.036 mL, 0.262 mmol) at 0° C. The cooling bath was removed, and the mixture was stirred for 30 minutes at room temperature. The (S)-2-(3-(2-(2-aminoethoxy) ethoxy)propanamido)-N1,N5-bis(14-azido-3,6,9,12-tetraoxatetradecyl)pentanediamide (0.117 g, 0.131 mmol) and DIEA (0.036 mL, 0.262 mmol) were added in DMF (0.1 mL) dropwise and the mixture was stirred at room temperature for 18 hours. The mixture was directly added onto a C18 column and purified by reverse-phase column chromatography using a gradient 0-50% acetonitrile in water (+0.1% formic acid). The residue obtained was re-purified by silica-gel column chromatography using a gradient 0-100% methanol in DCM. The residue was purified once again using a gradient 0-100% methanol in ethyl acetate to afford the titled compound in (13 mg, 9% yield). MS (ESI): m/z=1142.2 [M+Na]⁺.

BA-131 will be conjugated to an oligo sense strand according to general procedure type I or III.

### Example 9: BA-134 Conjugate

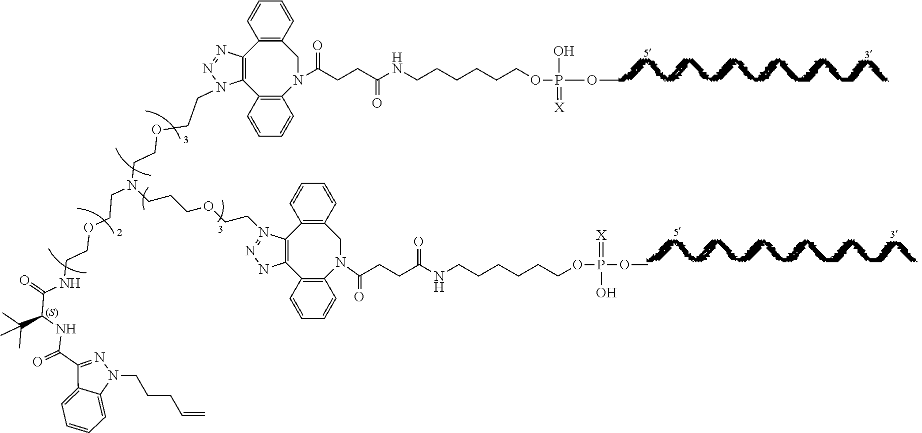

wherein X is O or S.

### BA-134: (S)—N-(1-azido-12-(2-(2-(2-(2-azidoethoxy)ethoxy)ethoxy)ethyl)-24,24-dimethyl-22-oxo-3,6,9,15,18-pentaoxa-12,21-diazapentacosan-23-yl)-1-(pent-4-en-1-yl)-1H-indazole-3-carboxamide

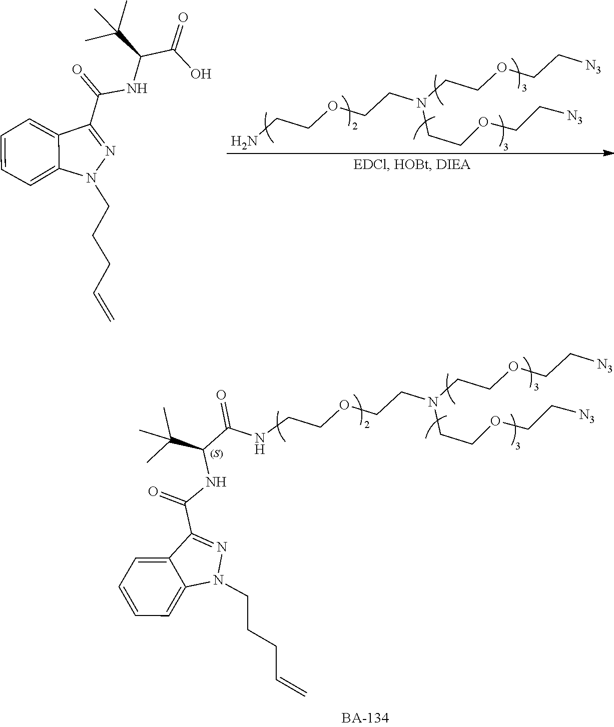

To a solution of (S)-3,3-dimethyl-2-(1-(pent-4-en-1-yl)-1H-indazole-3-carboxamido)butanoic acid (0.05 g, 0.146 mmol) and HOBt (0.026 g, 0.19 mmol) in DMF (0.7 mL), was added EDCI·HCl (0.042 g, 0.22 mmol) followed by Et₃N (0.074 g, 0.728 mmol) at 0° C. The cooling bath was removed, and the mixture was stirred for 30 mins at rt. 2-(2-(2-aminoethoxy)ethoxy)-N,N-bis(2-(2-(2-(2-azidoethoxy)ethoxy)ethoxy)ethyl)ethan-1-amine (0.104 g, 0.189 mmol) in DMF (0.1 mL) was added dropwise and the mixture was stirred at room temperature for 3 hours. The mixture was directly added onto a C18 column and purified by reverse-phase column chromatography using a gradient 0-50% acetonitrile in water (+0.1% formic acid) to afford the titled compound (37 mg, 29% yield) as a yellow oil. MS (ESI): m/z=876.7 [M+H]⁺.

BA-134 will be conjugated to an oligo sense strand according to general procedure type T or III.

### Example 10: BA-130 Conjugates

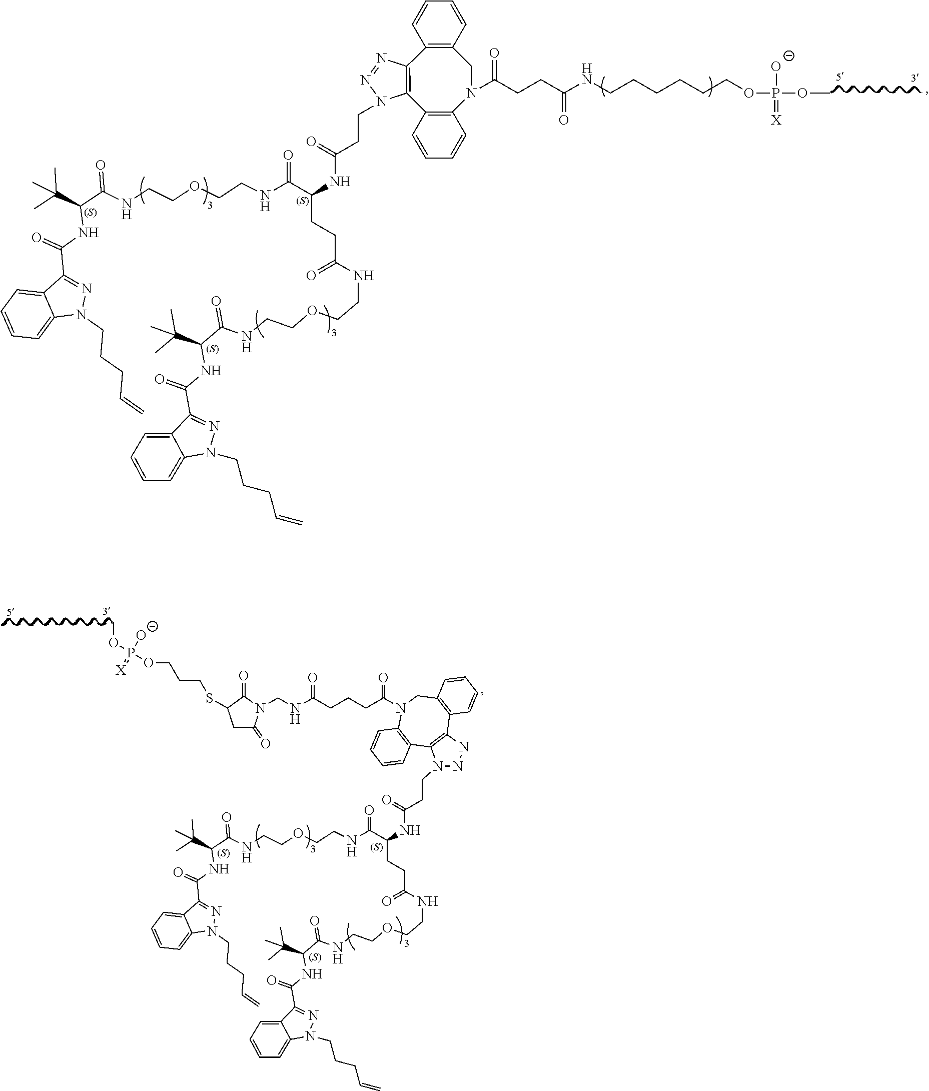

wherein X is O or S.

### BA-130 (S)-2-(3-azidopropanamido)-N1,N5-bis((S)-3-(tert-butyl)-1,4-dioxo-1-(1-(pent-4-en-1-yl)-1H-indazol-3-yl)-8,11,14-trioxa-2,5-diazahexadecan-16-yl)pentanediamide

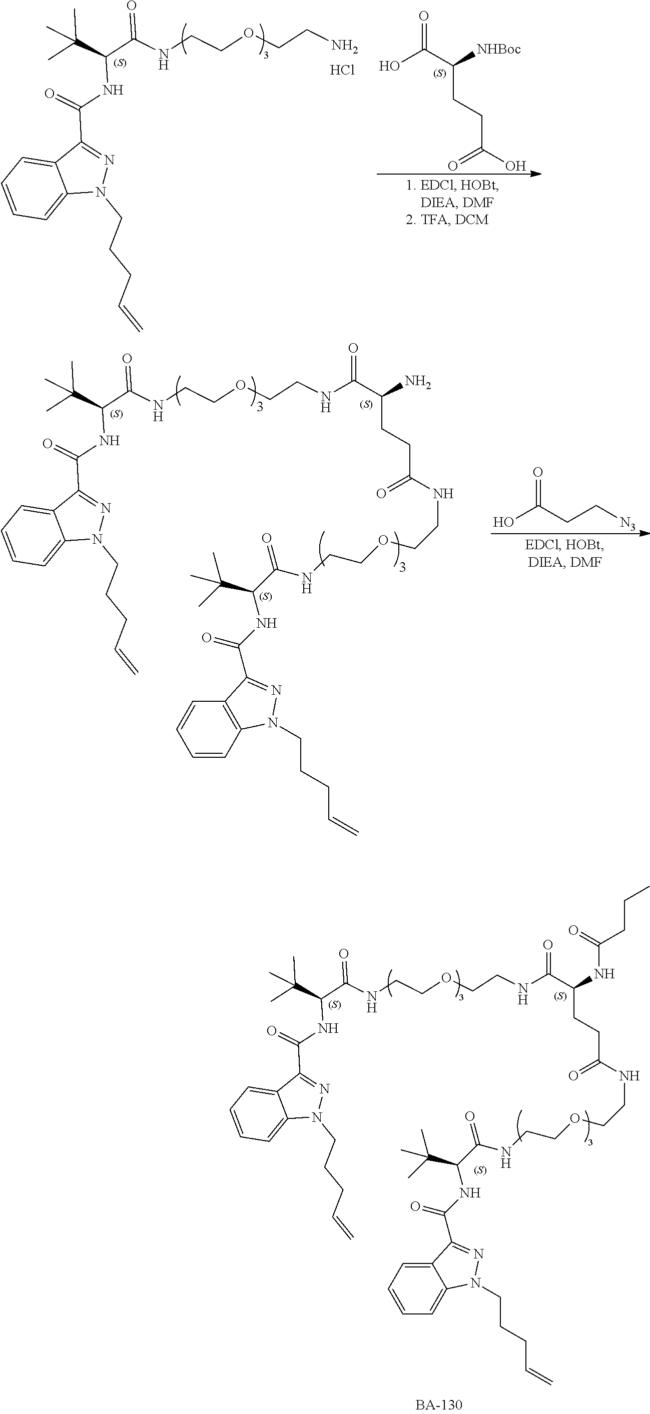

### Step 1: tert-butyl ((3S,19S,37S)-3,37-di-tert-butyl-1,4,18,22,36,39-hexaoxo-1,39-bis(1-(pent-4-en-1-yl)-1H-indazol-3-yl)-8,11,14,26,29,32-hexaoxa-2,5,17,23,35,38-hexaazanonatriacontan-19-yl)carbamate

To a solution of N-Boc-D-Glutamic acid (20 mg, 0.08 mmol) in DMF (0.5 mL) was added HOBt (24 mg, 0.18 mmol,), EDCI (62 mg, 0.324 mmol) then DIEA (71 mL, 0.404 mmol) at 0° C. The cooling bath was removed, and the mixture was stirred for 1 hour at room temperature. A solution of the (S)—N-(1-amino-15,15-dimethyl-13-oxo-3,6,9-trioxa-12-azahexadecan-14-yl)-1-(pent-4-en-1-yl)-1H-indazole-3-carboxamide hydrochloride (94 mg, 0.17 mmol) in DMF (0.25 mL) was added dropwise, followed by DIEA (42 mL, 0.243 mmol) and the mixture was stirred for 20 hours at room temperature. Water was added and the product was extracted with ethyl acetate (3×100 mL). The organic phases were combined, washed with aq sat NaHCO₃ (50 mL), then brine (50 mL), dried with Na₂SO₄, filtered then concentrated. The residue obtained was purified by silica-gel column chromatography using a gradient 0-10% methanol in ethyl acetate to afford the titled compound (40 mg, 40% yield) as a yellow oil. MS (ESI): m/z=1246.7 [M+H]⁺.

### Step 2: (S)-2-amino-N1,N5-bis((S)-3-(tert-butyl)-1,4-dioxo-1-(1-(pent-4-en-1-yl)-1H-indazol-3-yl)-8,11,14-trioxa-2,5-diazahexadecan-16-yl)pentanediamide

To a solution of tert-butyl ((3S,19S,37S)-3,37-di-tert-butyl-1,4,18,22,36,39-hexaoxo-1,39-bis(1-(pent-4-en-1-yl)-1H-indazol-3-yl)-8,11,14,26,29,32-hexaoxa-2,5,17,23,35,38-hexaazanonatriacontan-19-yl)carbamate (40 mg, 0.032 mmol) in anhydrous DCM (0.63 mL) at 0° C. was added TFA (0.3 mL) dropwise. The cooling bath was removed, and the mixture was stirred at room temperature for 3.5 hours then concentrated under reduced pressure to afford the titled compound (41 mg, 100% yield) which was used for the following step without further purification. MS (ESI): m/z=1147.2 [M+H]⁺.

### Step 3: (S)-2-(3-azidopropanamido)-N1,N5-bis((S)-3-(tert-butyl)-1,4-dioxo-1-(1-(pent-4-en-1-yl)-1H-indazol-3-yl)-8,11,14-trioxa-2,5-diazahexadecan-16-yl)pentanediamide

To a mixture of 3-azidopropanoic acid (5.0 mg, 0.048 mmol) in DCM (0.1 ml) was added HOBt (6.0 mg, 0.042 mmol), followed by EDCI (10 mg, 0.064 mmol) and DIEA (17 mL, 0.097 mmol) and the mixture was stirred at room temperature for 30 minutes. A mixture composed of (S)-2-amino-N1,N5-bis((S)-3-(tert-butyl)-1,4-dioxo-1-(1-(pent-4-en-1-yl)-1H-indazol-3-yl)-8,11,14-trioxa-2,5-diazahexadecan-16-yl)pentanediamide (40 mg, 0.032 mmol) and DIEA (7 mL, 0.039 mmol) in DCM (0.07 mL) was added dropwise to the activated acid. The mixture was stirred for 10 hours at room temperature then concentrated. The residue obtained was purified by silica-gel column chromatography using a gradient 0-10% methanol in ethyl acetate to afford the titled compound (33 mg, 83% yield). MS (ESI): m/z=1265.1.

BA-130 was conjugated to an oligo sense strand according to general procedure type 1. The product (MW: 8954.93 g/mol) was made with 95% purity and confirmed with HPLC and LCMS (m/z: 8953.37).

### Example 11: BA-180 Conjugates

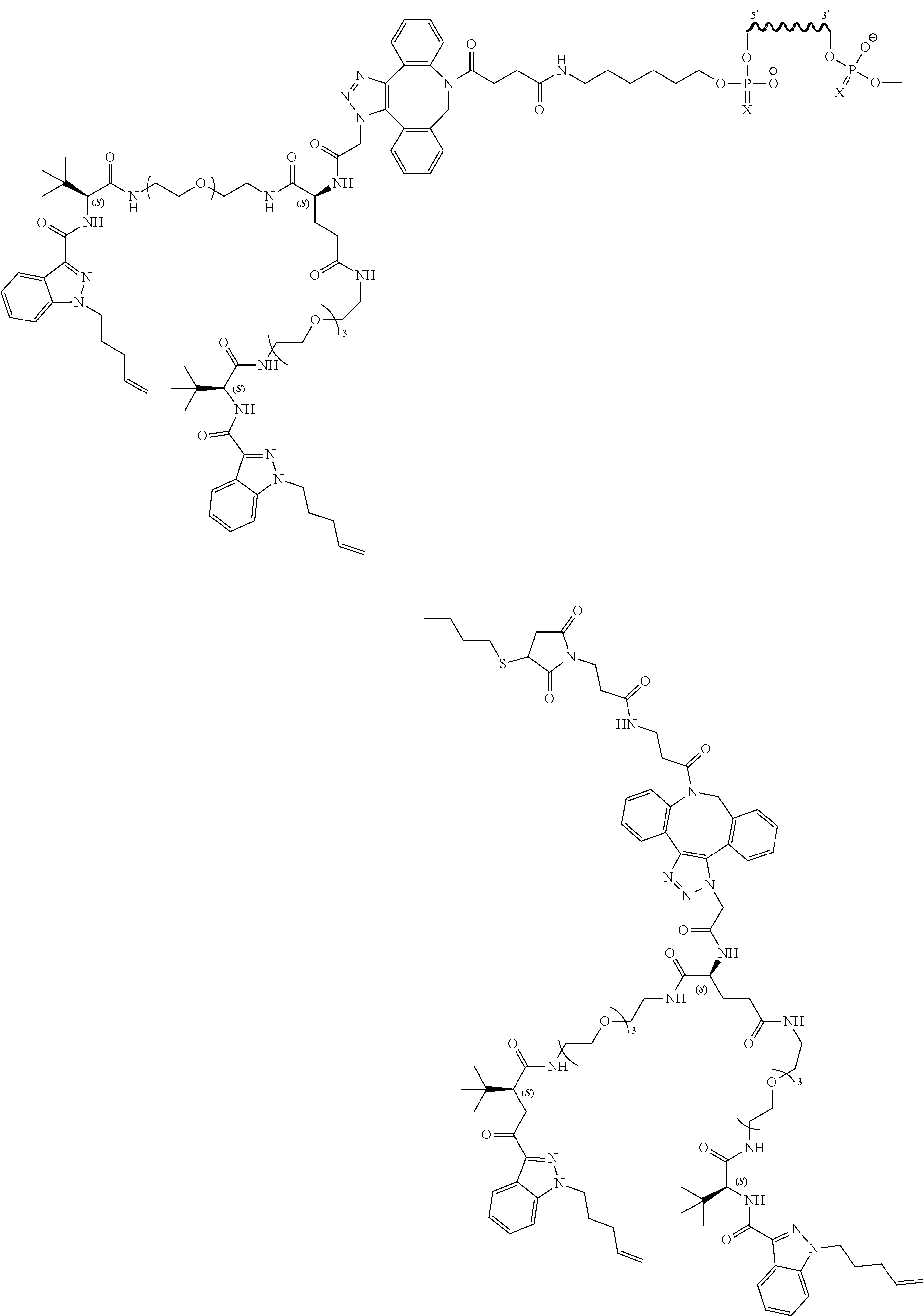

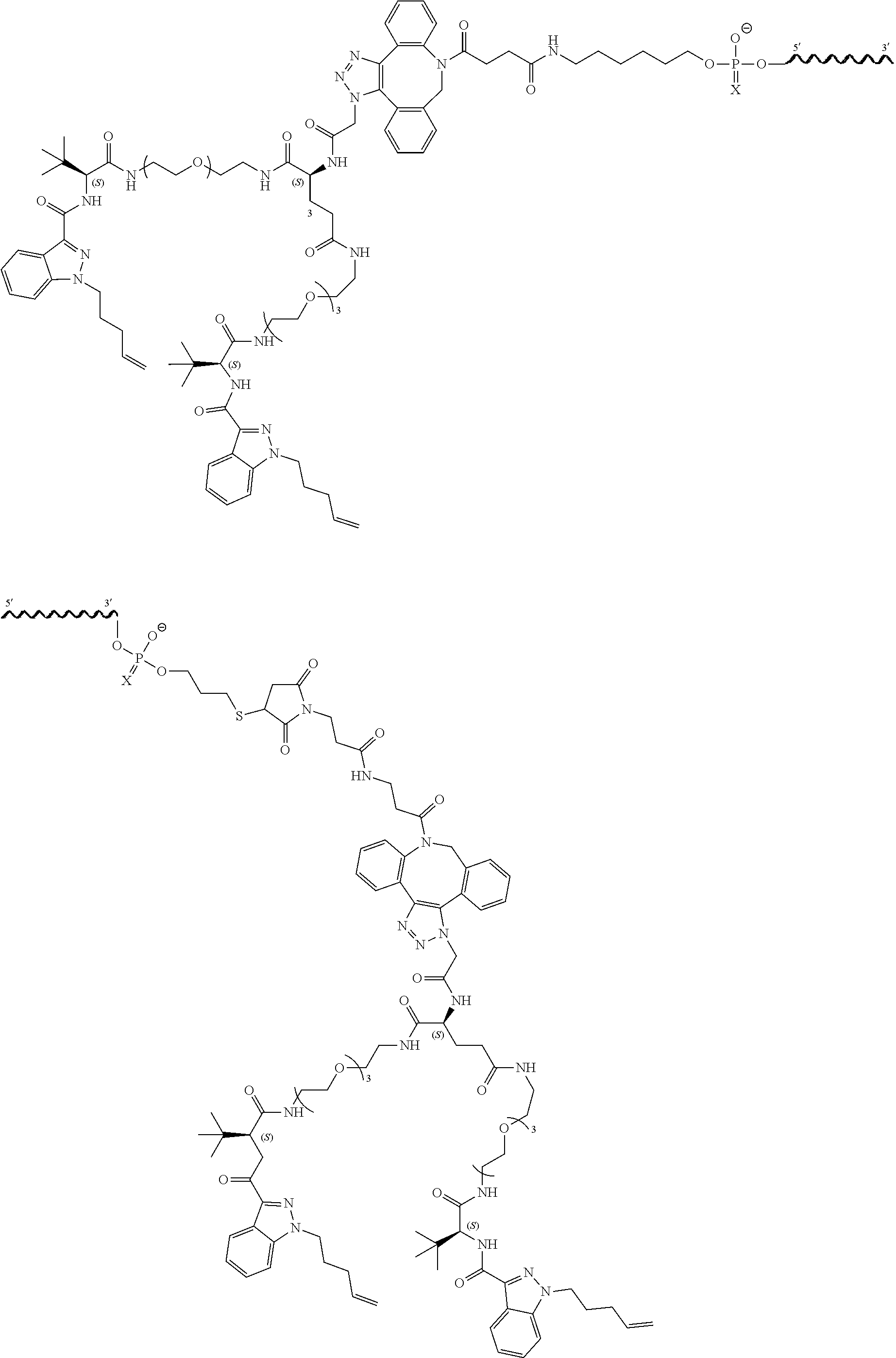

wherein X is O or S.

### BA-180: (S)-2-(2-azidoacetamido)-N1,N5-bis((S)-3-(tert-butyl)-1,4-dioxo-1-(1-(pent-4-en-1-yl)-1H-indazol-3-yl)-8,11,14-trioxa-2,5-diazahexadecan-16-yl)pentanediamide

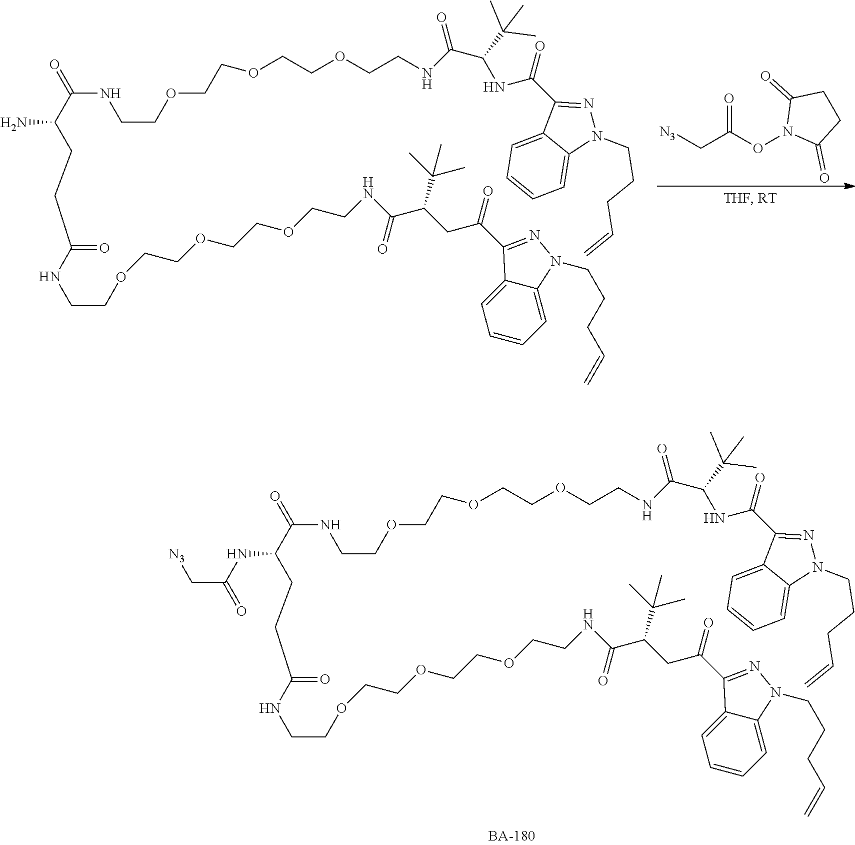

A suspension of NHS ester (155 mg, 0,785 mmol, 2 eq) and amine (S)-2-amino-N1,N5-bis((S)-3-(tert-butyl)-1,4-dioxo-1-(1-(pent-4-en-1-yl)-1H-indazol-3-yl)-8,11,14-trioxa-2,5-diazahexadecan-16-yl)pentanediamide (450 mg, 0.393 mmol, 1 eq) in THF (10 mL) was stirred for 12 h at RT. LCMS showed amide formation. Reaction mixture was diluted with Aq. saturated NaHCO₃ solution (20 ml), extracted with EtOAc 2×100 mL, combined extracts were dried over Na₂SO₄ and concentrated, and the crude was purified by flash chromatography using Ethyl acetate/MeOH, 0-20% as an eluent, pure fractions were combined and concentrated to obtain amide BA-180 (450 mg, 93%) as a white solid. NMR and LCMS m/z 1230 (M+1) are corresponding with the product. >95% HPLC and LCMS purity. ¹H NMR (499 MHz, DMSO-d₆) δ 8.35 (t, J=5.6 Hz, 2H), 8.24 (d, J=8.16 Hz, 1H) 8.16 (dt, J=8.2, 1.1 Hz, 2H), 8.05 (t, J=5.7 Hz, 1H), 7.85 (t, J=5.7 Hz, 1H), 7.77 (dt, J=8.6, 1.0 Hz, 2H), 7.59 (d, J=9.7 Hz, 2H), 7.46 (ddd, J=8.4, 6.8, 1.2 Hz, 2H), 7.28 (ddd, J=7.9, 6.8, 0.8 Hz, 2H), 5.82 (ddt, J=16.8, 10.2, 6.4 Hz, 2H), 5.06-4.98 (m, 2H), 4.98-4.94 (m, 2H), 4.56-4.46 (m, 6H), 4.27-4.20 (m, 1H), 3.85 (d, J=1.2 Hz, 2H), 3.51-3.40 (m, 20H), 3.39-3.29 (m, 7H), 3.24-3.11 (m, 6H), 2.13-2.00 (m, 6H), 1.97-1.91 (m, 3H), 1.90-1.79 (m, 1H), 1.78-1.62 (m, 1H), 0.97 (s, 18H).

BA-180 was conjugated to an oligo sense strand according to general procedure type I. The product (MW: 8940.90 g/mol) was made with 94% purity and confirmed with HPLC and LCMS (m/z: 8939.28).

BA-180 was bis-conjugated to an oligo sense strand according to general procedure type IIA. The product (MW: 10631.95 g/mol) was made with 97% purity and confirmed with HPLC and LCMS (m/z: 10629.92).

### Example 12: BA-153 Conjugate

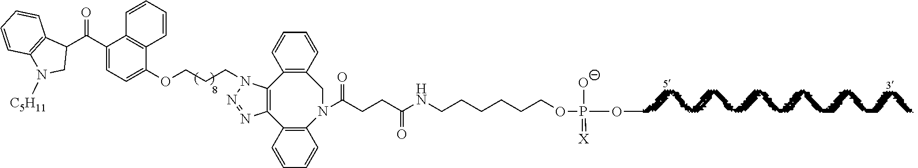

wherein X is O or S.

### BA-153: (4-((10-azidodecyl)oxy)naphthalen-1-yl)(1-pentyl-1H-indol-3-yl)methanone

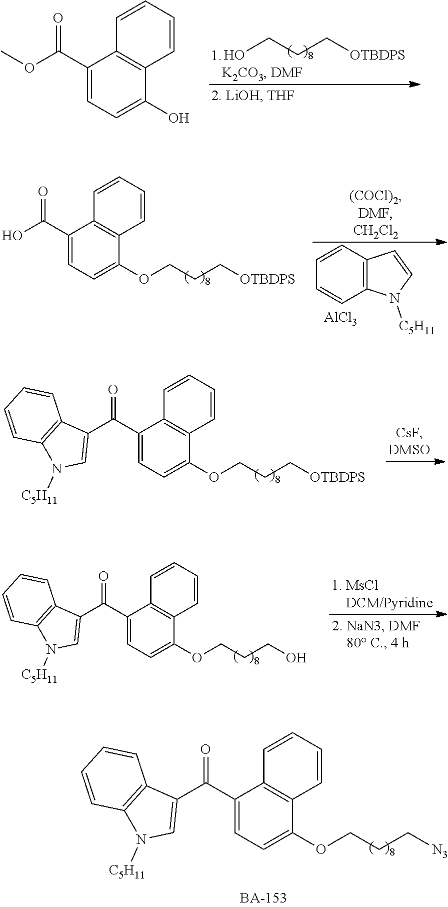

### Step 1: methyl 4-((10-((tert-butyldiphenylsilyl)oxy)decyl)oxy)-1-naphthoate

To a solution of [(10-bromodecyl)oxy](tert-butyl)diphenylsilane (36 g, 59 mmol, 70% purity) in CH₃CN (200 mL) were added methyl 4-hydroxynaphthalene-1-carboxylate (10 g, 49.454 mmol) and K₂CO₃ (10 g, 74 mmol). The mixture was stirred at 70° C. overnight under the nitrogen. The reaction mixture was quenched with water (200 mL) extracted with ethyl acetate (3×500 mL). The combined organic phase was dried over Na₂SO₄, filtered, and concentrated to afford the titled compound (13 g, 100% yield). The crude was used directly in the next step without further purification. MS (ESI): m/z=620 [M+Na]⁺.

### Step 2: 4-((10-((tert-butyldiphenylsilyl)oxy)decyl)oxy)-1-naphthoic acid

To a stirred solution of methyl 4-((10-((tert-butyldiphenylsilyl)oxy)decyl)oxy)-1-naphthoate (13.0 g, crude) in MeOH and H2O (v/v=1:1, 100 mL) was added NaOH (8.6 g, 590 mmol) in portions at room temperature. The resulting mixture was stirred for 24 h at 100° C. under nitrogen. The reaction was acidified by the addition of aqueous HCl solution (1N) at room temperature. The resulting mixture was extracted with EtOAc (3×100 mL). The combined organic layers were washed with brine, dried over anhydrous Na₂SO₄, filtered and concentrated. The residue was purified with silica gel (PE/EA) to afford the titled compound (1.8 g, 11% yield) as a yellow oil. 1H NMR (300 MHz, DMSO-d₆) δ 12.73 (s, 1H), 9.04 (d, J=8.6 Hz, 1H), 8.30-8.17 (m, 2H), 7.69-7.53 (m, 5H), 7.58-7.48 (m, 1H), 7.42 (d, J=5.9 Hz, 6H), 7.02 (dt, J=8.4, 2.1 Hz, 1H), 4.21 (dd, J=7.3, 4.1 Hz, 2H), 3.67-3.57 (m, 2H), 1.86 (s, 2H), 1.50 (p, J=6.5 Hz, 4H), 1.24 (s, 6H), 1.01-0.94 (m, 8H). MS (ESI): m/z=565 [M−H₂O+H]⁺.

### Step 3: (4-((10-((tert-butyldiphenylsilyl)oxy)decyl)oxy)naphthalen-1-yl)(1-pentyl-1H-indol-3-yl)methanone

To a stirred solution of 4-((10-((tert-butyldiphenylsilyl)oxy)decyl)oxy)-1-naphthoic acid (1.8 g, 4.1 mmol) in DCM (20 mL) was added DMF (several drops) in portions at room temperature. Oxalyl chloride (1.05 g, 8.4 mmol) in DCM was added dropwise at room temperature. The resulting mixture was stirred for 3 hours at room temperature under nitrogen. The reaction was concentrated under reduced pressure to obtain the acid chloride (1.8 g, crude) as a yellow oil which was used in the next step. To the solution of 1-pentyl-1H-indole (0.50 g, 2.66 mmol) in DCM (32 mL) was added dropwise Et₂AlCl (320.80 mg, 2.661 mmol, 1M in hexane) at 0° C. under the nitrogen. The mixture was stirred for 1 hour and the acid chloride prepared above (1.6 g, 2.7 mmol) in DCM (10 mL) was added dropwise. The reaction was allowed to warm to room temperature and stirred overnight. Added 1 M HCl aqueous solution (2 mL) dropwise to quench the reaction. The resulting mixture was extracted with DCM (2×100 mL). The combined organic layers were washed with brine, dried over anhydrous Na₂SO₄, filtered and concentrated. The residue was purified with silica gel (PE/EA) to afford the titled compound (1.14 g, yield: 55%) as a yellow oil. MS (ESI): m/z=752.8 [M+H]⁺.

### Step 4: (4-((10-hydroxydecyl)oxy)naphthalen-1-yl)(1-pentyl-1H-indol-3-yl)methanone

To the solution 3-[4-({10-[(tert-butyldiphenylsilyl)oxy]decyl}oxy)naphthalene-1-carbonyl]-1-pentylindole (1.1 g, 1.4 mmol) in DMSO (5 mL) was added cesium fluoride (0.33 g, 2.2 mmol,) at room temperature under the nitrogen. The reaction was allowed to be stirred overnight. The reaction was quenched by addition of water (1 mL). The resulting mixture was extracted with DCM (3×100 mL). The combined organic layers were washed with brine (50 mL), dried over anhydrous Na₂SO₄, filtered and was concentrated under reduced pressure. The residue was purified with silica gel (PE/EA) to afford the titled compound (0.67 g, 89% yield) as a white solid. 1H NMR (400 MHz, DMSO-d₆) δ 8.34-8.23 (m, 2H), 8.17-8.08 (m, 1H), 7.81 (s, 1H), 7.68 (d, J=7.9 Hz, 1H), 7.68-7.60 (m, 1H), 7.55 (qd, J=7.1, 3.5 Hz, 2H), 7.31 (pd, J=7.1, 1.4 Hz, 2H), 7.05 (d, J=8.0 Hz, 1H), 4.33 (t, J=5.1 Hz, 1H), 4.23 (dt, J=13.7, 6.7 Hz, 4H), 3.37 (td, J=6.5, 4.8 Hz, 2H), 1.91 (p, J=6.4 Hz, 2H), 1.73 (p, J=7.3 Hz, 2H), 1.56 (p, J=7.1 Hz, 2H), 1.45-1.03 (m, 18H), 0.80 (t, J=7.1 Hz, 3H). MS (ESI) n/z=514.3 [M+H]⁺.

### Step 5: 10-((4-(1-pentyl-1H-indole-3-carbonyl)naphthalen-1-yl)oxy)decyl methanesulfonate

Methanesulfonyl chloride (34 μL, 438 mol) was added dropwise to a solution of (4-((10-hydroxydecyl)oxy)naphthalen-1-yl)(1-pentyl-1H-indol-3-yl)methanone (150 mg, 292 μmol) in DCM (3 mL) and pyridine (3 mL) at 0° C. The reaction mixture was stirred at room temperature for 4 hours and quenched by addition of water and extracted with DCM. The organic layer was washed with a saturated solution of NaHCO₃ and brine. The combined organic layer was dried over MgSO₄, filtered and concentrated to afford the titled compound (167 mg, 97% yield) as a yellow oil, was used without further purification for the following step. MS (ESI): m/z=592.5 [M+H]⁺.

### Step 6: (4-((10-azidodecyl)oxy)naphthalen-1-yl)(1-pentyl-1H-indol-3-yl)methanone

To a solution of 10-((4-(1-pentyl-1H-indole-3-carbonyl)naphthalen-1-yl)oxy)decyl methanesulfonate (0.15 g, 0.253 mmol) in DMF (4 mL) was added NaN₃ (0.036 g, 0.558 mmol) and the mixture was stirred for 4 hours at 80° C., cooled down to rt and partitioned between ethyl acetate (30 mL) and water (10 mL). The organic phase was separated, and the aqueous phase was extracted with ethyl acetate (2×20 mL). The combined organic layers were washed with brine (2×20 mL), dried with Na₂SO₄, filtered and concentrated. The residue was purified by reverse-phase column chromatography using a gradient 0-100% water in acetonitrile (+0.1% formic acid) to afford the titled compound in (116 mg, 85% yield). MS: (ESI) m/z=539.5 [M+H]⁺.

BA-153 was conjugated to an oligo sense strand according to general procedure type I. The product (MW: 7838.81 g/mol) was made with 95% purity and confirmed with HPLC and LCMS (m/z: 7837.52).

### Example 13: BA-163 Conjugate

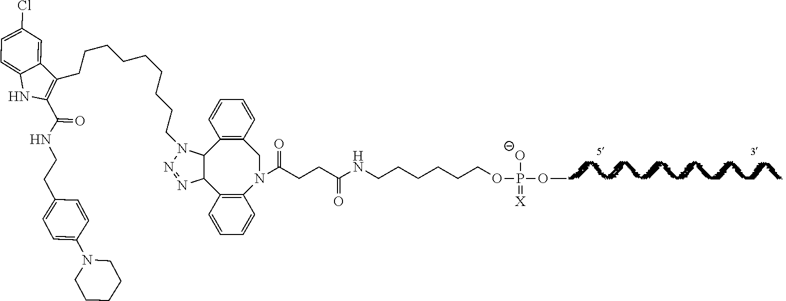

wherein X is O or S.

### BA-163 3-(9-azidononyl)-5-chloro-N-(4-(piperidin-1-yl)phenethyl)-1H-indole-2-carboxamide

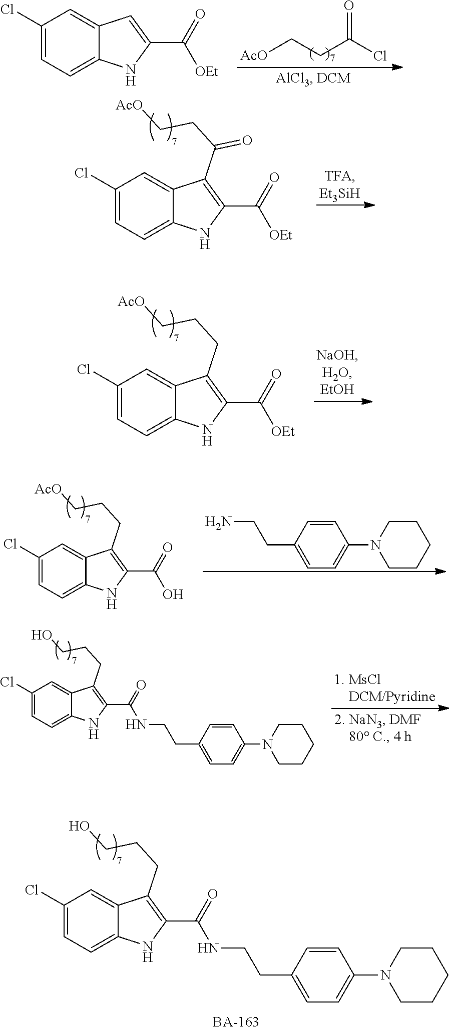

### Step 1: ethyl 3-(9-acetoxynonanoyl)-5-chloro-1H-indole-2-carboxylate

To a stirred solution of ethyl 5-chloro-1H-indole-2-carboxylate (1.4 g, 6.26 mmol) in DCM (15 mL) was added AlCl₃ (0.92 g, 6.89 mmol) in portions at room temperature. To the above mixture was added 9-chloro-9-oxononyl acetate (1.62 g, 6.89 mmol) in DCM (12 mL) dropwise at room temperature. The resulting mixture was stirred for additional overnight at room temperature. The reaction was quenched by the addition of water (200 mL) at room temperature. The resulting mixture was extracted with EtOAc (2×100 mL). The combined organic layers were washed with brine (100 mL), dried over anhydrous Na₂SO₄, filtered, and concentrated. The residue was purified by silica gel column chromatography, eluted with PE/EA (1:3) to afford the titled compound (880 mg, 33.3% yield) as a brown yellow oil. MS (ESI): m/z=422.2 [M+H]⁺.

### Step 2: ethyl 3-(9-acetoxynonyl)-5-chloro-1H-indole-2-carboxylate

To a stirred solution of ethyl 3-[9-(acetyloxy)nonanoyl]-5-chloro-1H-indole-2-carboxylate (880 mg, 2.1 mmol) and Et₃SiH (970 mg, 8.3 mmol) was added TFA (8 mL, 107.705 mmol) dropwise at room temperature. The resulting mixture was stirred for 1 hour at room temperature. The reaction was quenched by the addition of sat. NaHCO₃ (aq.) (200 mL). The resulting mixture was extracted with EtOAc (2×100 mL). The combined organic layers were washed with brine (100 mL), dried over anhydrous Na₂SO₄, filtered and concentrated. The residue was purified by silica gel column chromatography, eluted with PE/EA (3:1) to afford the titled compound (500 mg, 59% yield) as a brown-yellow oil. MS (ESI): m/z=408.1 [M+H]⁺.

Step 3: 5-chloro-3-(9-hydroxynonyl)-1H-indole-2-carboxylic acid To a stirred solution of ethyl 3-(9-acetoxynonyl)-5-chloro-1H-indole-2-carboxylate (700 mg, 1.7 mmol) in EtOH (3 mL) was added NaOH (274 mg, 6.9 mmol) in H₂O (3 mL) dropwise at room temperature. The resulting mixture was stirred for overnight. The reaction was quenched by the addition of water (10 mL) at room temperature. The mixture was acidified to pH 2 with 1N HCl (aq.). The resulting mixture was extracted with EtOAc (2×100 mL). The combined organic layers were dried over anhydrous Na₂SO₄, filtered and concentrated. The residue was purified by reverse flash chromatography with the following conditions: column, C18 silica gel; mobile phase, MeCN in water, 13% to 100% gradient in 20 min; detector, UV 254 nm, to afford the titled compound (427 mg, 74% yield) as a white solid. ¹H NMR (400 MHz, DMSO-d₆) δ 11.56 (s, 1H), 7.67 (d, J=2.0 Hz, 1H), 7.39 (d, J=8.7 Hz, 1H), 7.22 (dd, J=8.7, 2.1 Hz, 1H), 4.42 (s, 1H), 3.36 (t, J=6.5 Hz, 2H), 3.01 (dd, J=8.5, 6.5 Hz, 2H), 1.56 (h, J=6.6 Hz, 2H), 1.49-1.07 (m, 12H).

MS (ESI): m/z=338.1 [M+H]⁺.

Step 4: 5-chloro-3-(9-hydroxynonyl)-N-(4-(piperidin-1-yl)phenethyl)-1H-indole-2-carboxamide Add HATU (338 mg, 0.89 mmol) and DIPEA (0.31 mL, 1.78 mmol) to a solution of 5-chloro-3-(9-hydroxynonyl)-1H-indole-2-carboxylic acid (200 mg, 0.59 mmol) and 4-(1-piperidinyl)benzeneethanamine (156 mg, 0.76 mmol) in DMF (2.4 mL) at 0° C. and stir at room temperature for 50 minutes. The reaction mixture was poured into EtOAc-hexanes (9:1, 25 mL) and washed with saturated NaHCO₃ (25 mL). The aqueous layer was extracted with EtOAc-Hexanes (9:1, 25 mL). The combined organic extracts were washed with 10% saturated NaCl (2×25 mL), and with brine (25 mL). The organic layer was dried over anhydrous sodium sulfate, filtered, and concentrated under reduced pressure. The residue was purified by FCC on silica gel (12 g silicycle, 0 to 60% EtOAc/hexanes;) to afford titled compound (230 mg, 74% yield) as a white solid. MS (ESI): m/z=524.6 [M+H]⁺.

### Step 5: 9-(5-chloro-2-((4-(piperidin-1-yl)phenethyl)carbamoyl)-1H-indol-3-yl)nonyl methanesulfonate

Methanesulfonyl chloride (0.07 mL, 0.88 mmol) was added dropwise to a solution of 5-chloro-3-(9-hydroxynonyl)-N-(4-(piperidin-1-yl)phenethyl)-1H-indole-2-carboxamide (230 mg, 0.44 mmol) in anhydrous DCM (4.4 mL) and pyridine (0.44 mL, 5.5 mmol) at 0° C. and stir for 75 minutes at room temperature. The reaction mixture was poured into DCM (50 mL) and saturated NaHCO₃ (25 mL). The aqueous layer was extracted with DCM (50 mL). The combined organic extracts were dried over anhydrous sodium sulfate, filtered and concentrated to afford the titled compound (264 mg, 100% yield) as a yellow oil which was carried forward without further purification. MS (ESI): m/z=602 [M+H]⁺.

### Step 6: 3-(9-azidononyl)-5-chloro-N-(4-(piperidin-1-yl)phenethyl)-1H-indole-2-carboxamide

The mesylate, 9-(5-chloro-2-((4-(piperidin-1-yl)phenethyl)carbamoyl)-1H-indol-3-yl)nonyl methanesulfonate (264 mg, 0.44 mmol) was dissolved in DMF (4.4 mL), sodium azide (142 mg, 2.2 mmol) was added and the reaction was heated to 80° C. for 16 hours. The reaction mixture was poured into EtOAc-Hexanes (50 mL, 9:1) and washed with saturated NaHCO₃ (50 mL). The aqueous layer was extracted with EtOAc-Hexanes (50 mL, 9:1). The combined organic layers were washed with 20% sat'd NaCl (2×50 mL) and with brine (50 mL). The organic layer was dried over anhydrous sodium sulfate, filtered, and concentrated under reduced pressure. The residue was purified by FCC on silica gel (10 g 20 micron, 0 to 40% EtOAc/hexanes) to afford the product as a white solid that turned orange. Repurified by FCC on silica gel (12 g HP, 0 to 40% EtOAc/Hexanes) to afford the titled compound (188 mg, 78% yield) as a white solid. MS (ESI-ve mode): m/z=547.2 [M−H]⁻.

BA-163 was conjugated to an oligo sense strand according to general procedure type I. The product (MW: 8259.71 g/mol) was made with 99% purity and confirmed with HPLC and LCMS (m/z: 8258.88).

### Example 14: BA-176 Conjugates

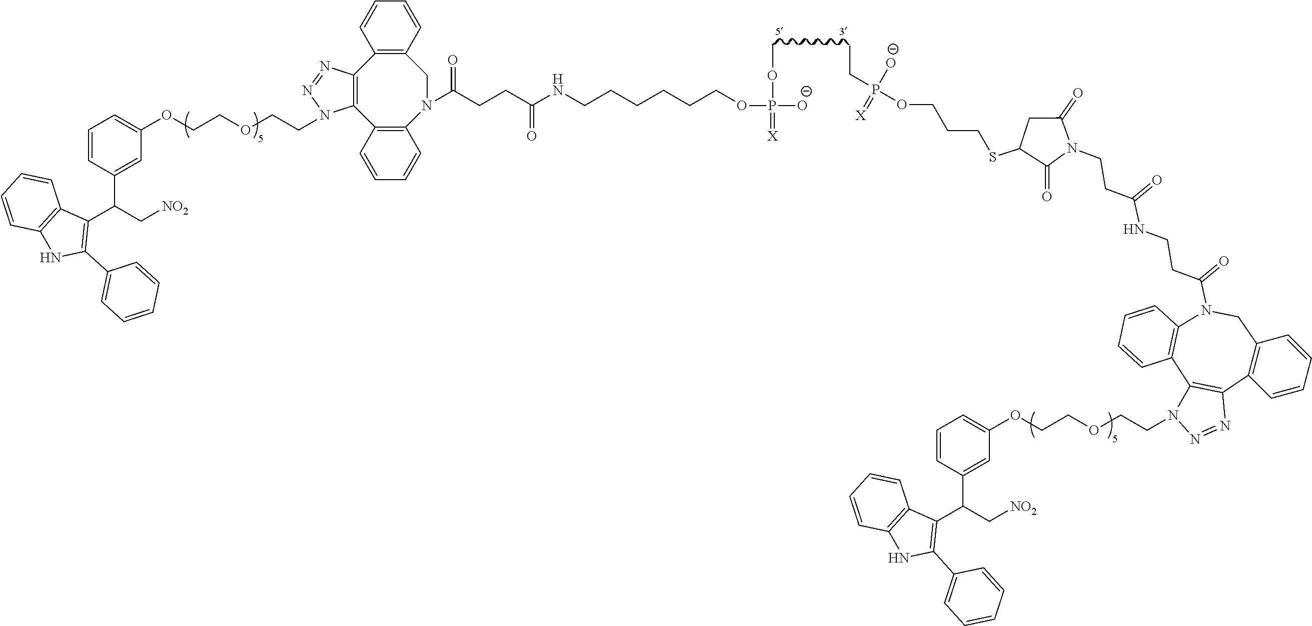

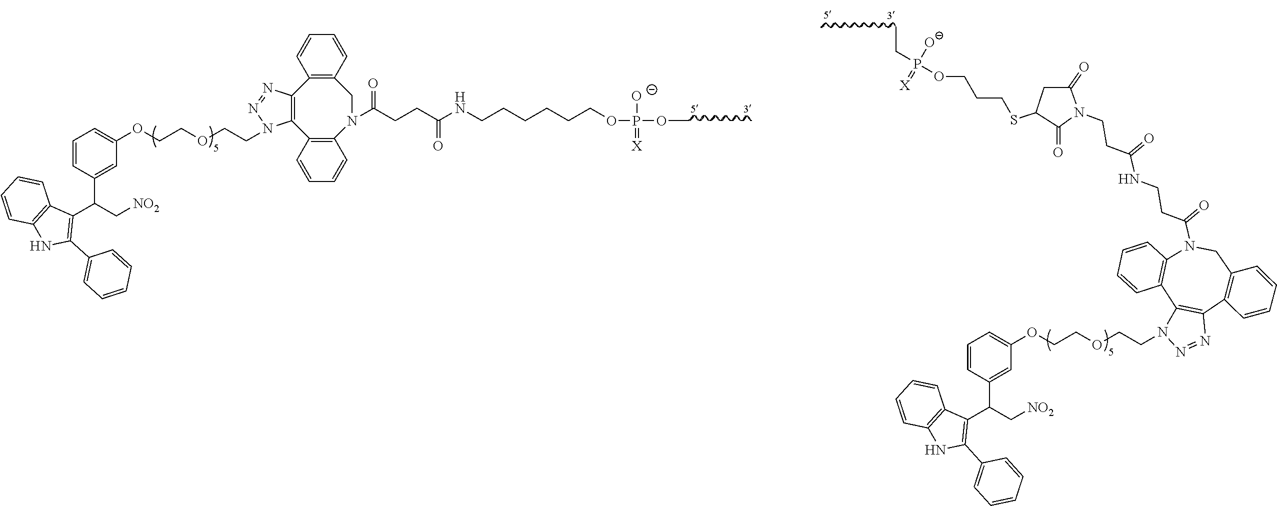

wherein X is O or S.

### BA-176: 3-(1-(3-((17-azido-3,6,9,12,15-pentaoxaheptadecyl)oxy)phenyl)-2-nitroethyl)-2-phenyl-1H-indole

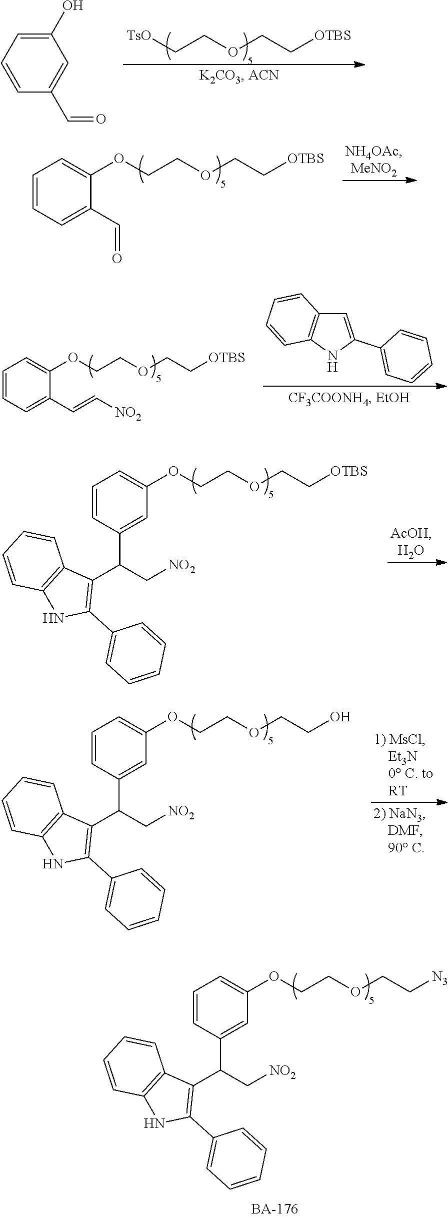

### Step 1: 2-((2,2,3,3-tetramethyl-4,7,10,13,16,19-hexaoxa-3-silahenicosan-21-yl)oxy)benz aldehyde

To a stirred solution of 3-hydroxybenzaldehyde (3 g, 24.565 mmol) and K₂CO₃ (5.09 g, 37 mmol) in MeCN (120 mL) was added 2,2,3,3-tetramethyl-4,7,10,13,16,19-hexaoxa-3-silahenicosan-21-yl 4-methylbenzenesulfonate (13.53 g, 24.57 mmol) in portions at room temperature under argon atmosphere. The resulting mixture was stirred for overnight at 70° C. under argon atmosphere. The reaction was quenched by the addition of water (100 mL) at room temperature. The resulting mixture was extracted with EtOAc (3×500 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:1) to the titled compound (11.7 g, 95% yield) as a colorless oil. 1H-NMR (300 MHz, DMSO-d₆) δ 9.95 (s, 1H), 7.54-7.37 (m, 3H), 7.32-7.20 (m, 1H), 4.21-4.11 (m, 2H), 3.83-3.70 (m, 2H), 3.64 (dd, J=5.7, 4.6 Hz, 2H), 3.58 (ddd, J=5.8, 3.4, 1.1 Hz, 2H), 3.55-3.51 (m, 2H), 3.49 (s, 14H), 3.44-3.38 (m, 3H), 2.39 (s, 1H), 0.82 (d, J=1.5 Hz, 11H). MS (ESI): m/z 500.3 [M+H]⁺.

### Step 2: (E)-2,2,3,3-tetramethyl-21-(2-(2-nitrovinyl)phenoxy)-4,7,10,13,16,19-hexaoxa-3-silahenicosane

To a stirred solution of 3-[(2,2,3,3-tetramethyl-4,7,10,13,16,19-hexaoxa-3-silahenicosan-21-yl)oxy]benzaldehyde (9.2 g, 18 mmol) in nitromethane (92 mL) was added NH₄OAc (1.77 g, 23.0 mmol) at room temperature under argon atmosphere. The resulting mixture was stirred for 4 hours at 60° C. under argon atmosphere. The reaction was quenched by the addition of water (200 mL) at room temperature. The resulting mixture was extracted with EtOAc (2×200 mL). The combined organic layers were washed with brine (100 mL), dried over anhydrous MgSO₄, filtered and was concentrated. The residue was purified by silica gel column chromatography, eluted with PE/EA (2:1) to afford the titled compound (3.5 g, 35% yield) as a light yellow oil. 1H NMR (300 MHz, DMSO-d₆) δ 8.34-7.90 (m, 2H), 7.57-7.27 (m, 3H), 7.08 (dt, J=7.1, 2.3 Hz, 1H), 4.23-4.07 (m, 2H), 3.82-3.60 (m, 5H), 3.60-3.44 (m, 18H), 3.41 (t, J=5.1 Hz, 2H), 3.29 (s, 1H), 0.82 (s, 10H). MS (ESI): m/z=543.3 [M+H]⁺.

### Step 3: 3-(2-nitro-1-(3-((2,2,3,3-tetramethyl-4,7,10,13,16,19-hexaoxa-3-silahenicosan-21-yl)oxy)phenyl)ethyl)-2-phenyl-1H-indole

A mixture of (E)-2,2,3,3-tetramethyl-21-(2-(2-nitrovinyl)phenoxy)-4,7,10,13,16,19-hexaoxa-3-silahenicosane (2.97 g, 5.46 mmol) and 2-phenyl-1H-indole (1.37 g, 7.10 mmol) and ammonium trifluoroacetate (0.93 g, 7.10 mmol) in EtOH (50 mL) was stirred for 3.5 hours at 100° C. under argon atmosphere. The reaction was quenched with NaHCO₃ at 0° C. The resulting mixture was extracted with EtOAc. The combined organic layers were washed with brine, dried over anhydrous Na₂SO₄, filtered and concentrated. The residue was purified by silica gel column chromatography, eluted with PE/EA (1:1) to afford the titled compound (3.14 g, 78% yield) as a yellow oil. 1H-NMR (300 MHz, DMSO-d₆) δ 11.41 (s, 1H), 7.63 (d, J=8.0 Hz, 1H), 7.57-7.27 (m, 6H), 7.27-6.67 (m, 7H), 5.73 (s, 2H), 5.52 (dd, J=13.2, 7.4 Hz, 1H), 5.39 (dd, J=13.1, 9.0 Hz, 1H), 5.14 (t, J=8.2 Hz, 1H), 4.05-3.93 (m, 3H), 3.65 (td, J=5.2, 3.2 Hz, 5H), 3.60-3.43 (m, 19H), 3.40 (dd, J=5.7, 4.6 Hz, 3H), 3.30 (s, 3H), 1.96 (s, 1H), 1.24-1.07 (m, 2H). MS (ESI): m/z=736.4 [M+H]⁺.

### Step 4: 17-(3-(2-nitro-1-(2-phenyl-1H-indol-3-yl)ethyl)phenoxy)-3,6,9,12,15-pentaoxahepta decan-1-ol

A mixture of 3-(2-nitro-1-{3-[(2,2,3,3-tetramethyl-4,7,10,13,16,19-hexaoxa-3-silahenicosan-21-yl)oxy]phenyl}ethyl)-2-phenyl-1H-indole (3.14 g, 4.26 mmol) and acetic acid (31 mL) in water (31 mL) was stirred for overnight at room temperature under argon atmosphere. The reaction was quenched with NaHCO₃ at 0° C. The resulting mixture was extracted with EtOAc (2×100 mL). The combined organic layers were washed with brine (100 mL), dried over anhydrous Na₂SO₄, filtered and concentrated. The residue was purified by reversed-phase flash chromatography with the following conditions: column, C18 silica gel; mobile phase, MeCN in Water, 5% to 95% gradient in 30 min; detector, UV 254 nm to afford the titled compound (2.01 g, 76% yield) as a yellow oil. 1H-NMR (300 MHz, DMSO-d₆) δ 11.45 (s, 1H), 7.66 (d, J=8.0 Hz, 1H), 7.54 (d, J=5.8 Hz, 4H), 7.46 (dq, J=8.5, 2.6 Hz, 1H), 7.39 (d, J=8.0 Hz, 1H), 7.16 (dt, J=24.7, 7.8 Hz, 2H), 6.99 (t, J=7.5 Hz, 1H), 6.92-6.77 (m, 3H), 5.55 (dd, J=13.2, 7.4 Hz, 1H), 5.42 (dd, J=13.2, 9.0 Hz, 1H), 5.17 (t, J=8.1 Hz, 1H), 4.57 (t, J=5.4 Hz, 1H), 4.00 (dd, J=5.8, 3.5 Hz, 2H), 3.77-3.44 (m, 21H), 3.40 (t, J=4.8 Hz, 2H). MS (ESI): m/z=622.3 [M+H]⁺.

### Step 5: 3-(1-(3-((17-azido-3,6,9,12,15-pentaoxaheptadecyl)oxy)phenyl)-2-nitroethyl)-2-phenyl-1H-indole

To a solution of 17-(3-(2-nitro-1-(2-phenyl-1H-indol-3-yl)ethyl)phenoxy)-3,6,9,12,15-penta oxaheptadecan-1-ol (0.25 g, 0.40 mmol) in anhydrous DCM (4 mL), Et₃N (70 mL, 0.60 mmol) was added followed by the MsCl (34 mL, 0.44 mmol) at 0° C. and slowly warmed up to room temperature. After 1 hr, the reaction mixture was quenched with saturated NaHCO₃, extracted with dichloromethane (3×20 mL). The combined organic layer was washed with brine and dried over Na₂SO₄ filtered and concentrated. Crude mixture was used in the next step without further purification. Dissolved the mesylate prepared above in DMF (4 mL), NaN₃ (89 mg, 1.2 mmol) was added at room temperature and the mixture was heated to 90° C. After 16 hours, the mixture was cooled down to room temperature. Mixture was diluted with water, extracted with ethyl acetate (3×50 mL), the combined organic layer was washed with brine solution, and dried over Na₂SO₄ filtered and concentrated. The residue was purified by flash chromatography (25 g, 20 micron Biotage column) using hexanes/EtOAc, 0-20% 5 CV then 20% 10 CV as an eluent to obtain the titled compound (131 mg, 50% yield) as a brown oil. 1H NMR (500 MHz, DMSO-d₆) δ 11.44 (s, 1H), 7.66 (d, J=10 Hz, 1H), 7.56-7.53 (m, 4H), 7.48-7.46 (m, 1H), 7.38 (d, J=10 Hz, 1H), 7.20 (t, J=15 Hz, 1H), 7.12 (t, J=15 Hz, 1H), 6.99 (t, J=10 Hz, 1H), 6.87-6.78 (m, 3H), 5.56-5.52 (m, 1H), 5.44-5.40 (m, 1H), 5.18-5.15 (m, 1H), 4.01-3.99 (m, 2H), 3.69-3.67 (m, 2H), 3.58-3.50 (m, 19H), 3.43-3.37 (m, 22H). MS (ESI, negative mode): m/z=646.4 [M−H]⁻.

BA-176 was conjugated to an oligo sense strand according to general procedure type I. The product (MW: 8359.14 g/mol) was made with 99% purity and with HPLC and LCMS (m/z: 8357.61).

BA-176 was bis-conjugated to an oligo sense strand according to general procedure type IIA. The product (MW: 9468.43 g/mol) was made with 97% purity and confirmed with HPLC and LCMS (m/z: 9466.70).

### Example 15: BA-177 Conjugates

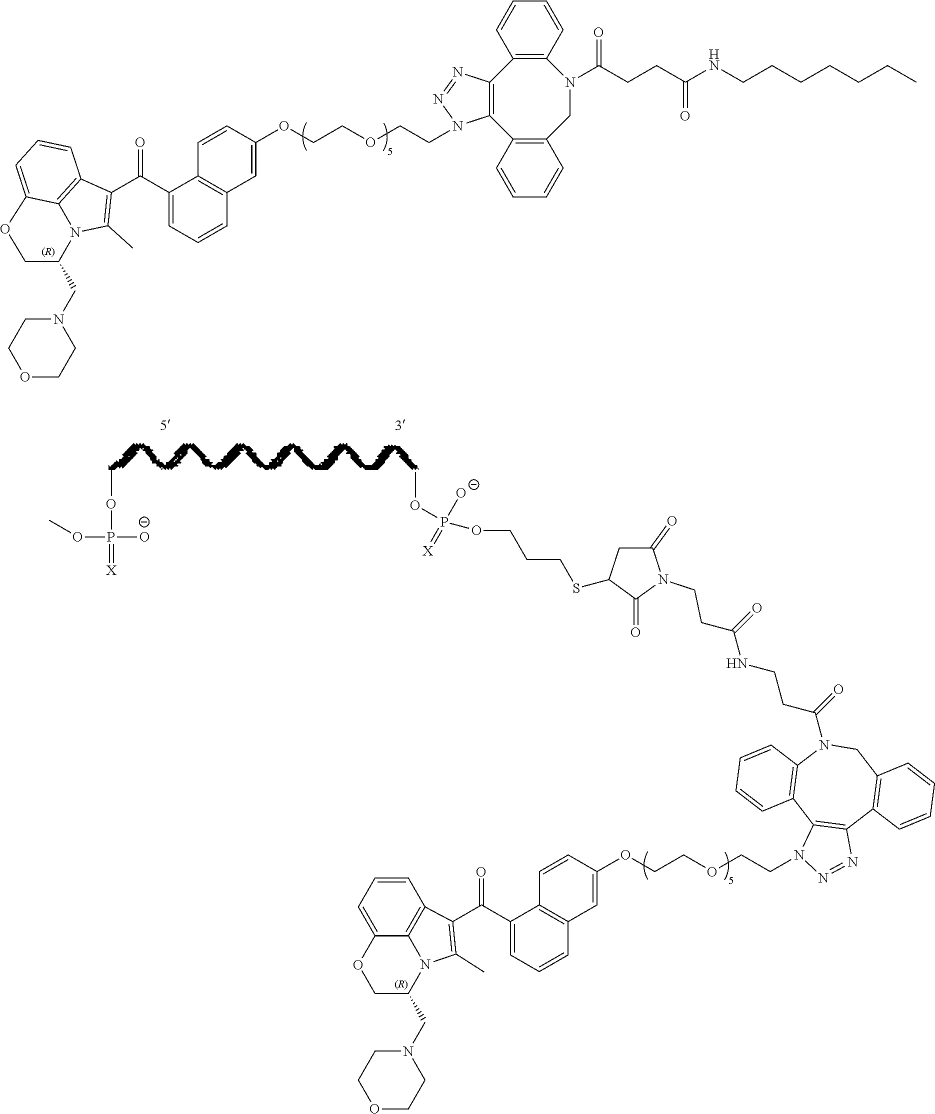

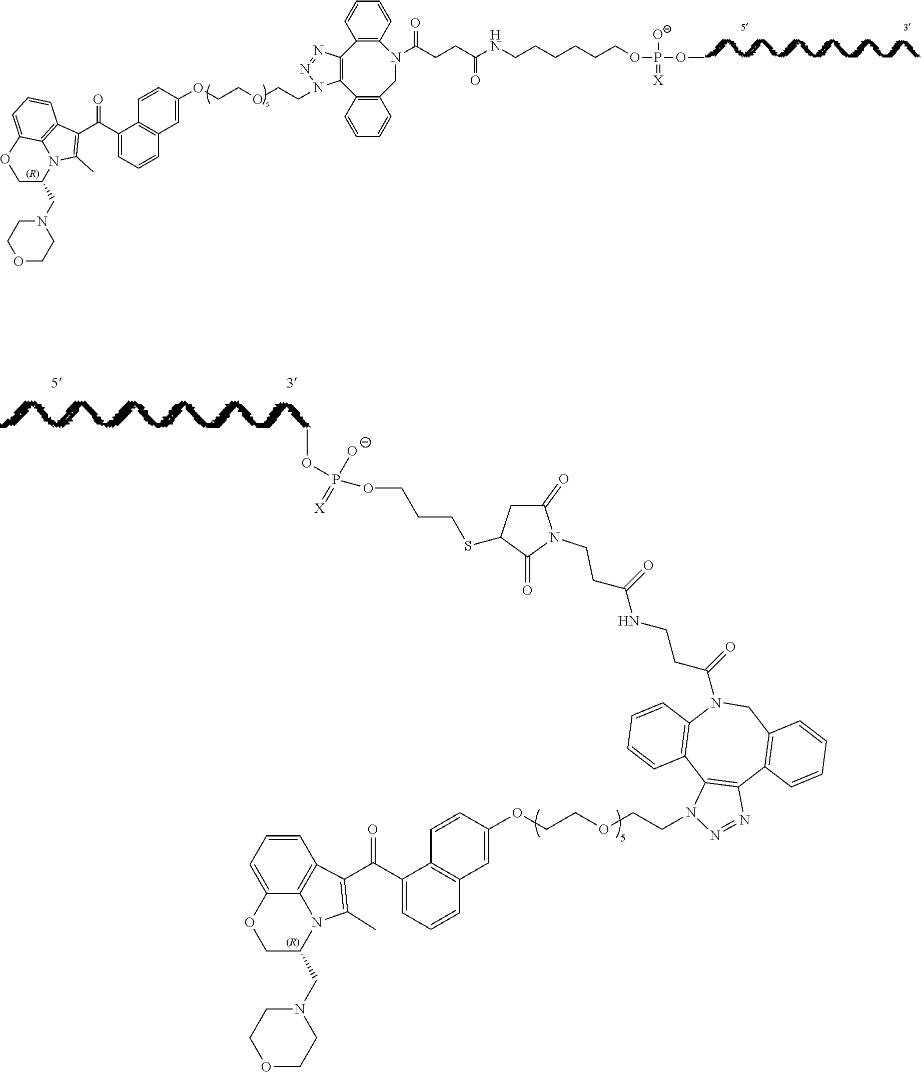

wherein X is O or S.

### BA-177: (R)-(6-((17-azido-3,6,9,12,15-pentaoxaheptadecyl)oxy)naphthalen-1-yl)(5-methyl-3-(morpholinomethyl)-2,3-dihydro-[1,4]oxazino[2,3,4-hi]indol-6-yl)methanone

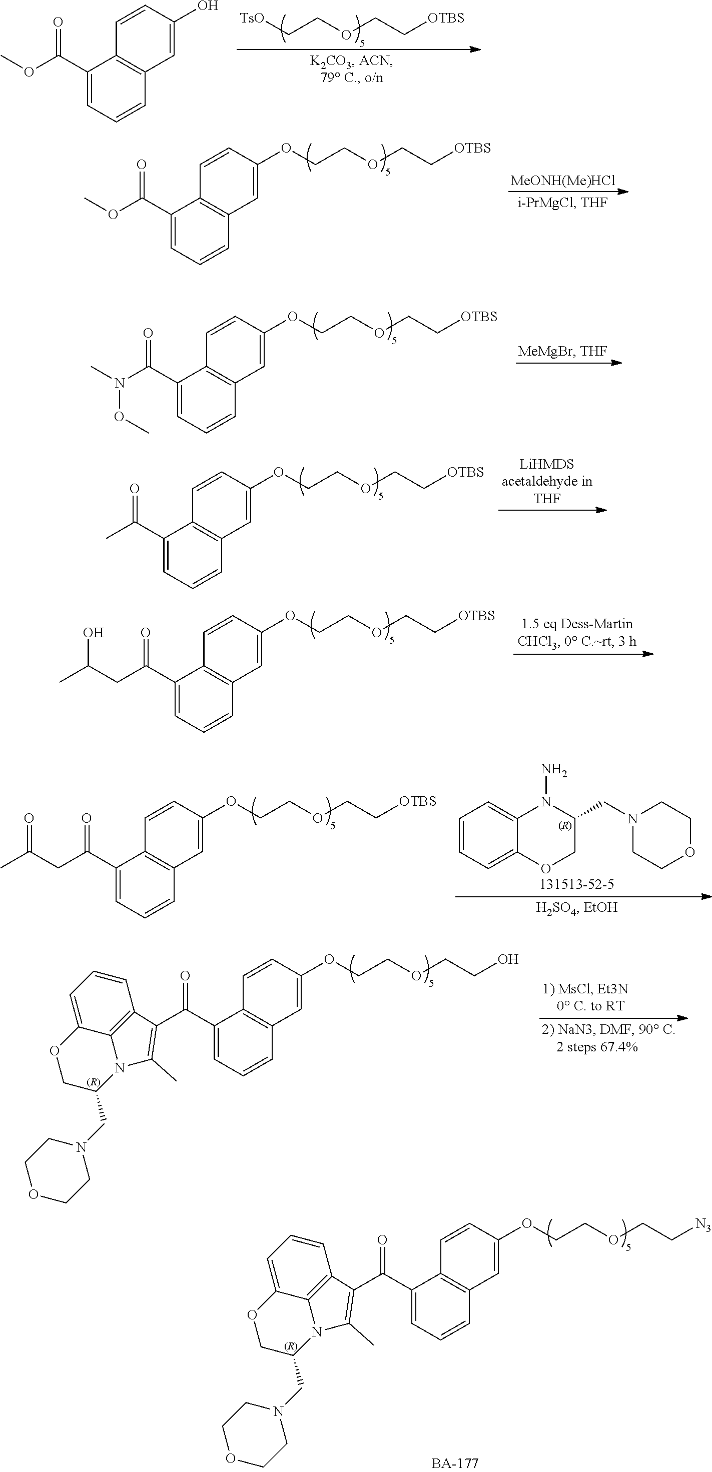

### Step 1: methyl 6-((2,2,3,3-tetramethyl-4,7,10,13,16,19-hexaoxa-3-silahenicosan-21-yl)oxy)-1-naphthoate

To a stirred solution of methyl 6-hydroxynaphthalene-1-carboxylate (5.0 g, 25 mmol) in ACN (150 mL) were added K₂CO₃ (5.13 g, 37.1 mmol) and 2,2,3,3-tetramethyl-4,7,10,13,16,19-hexaoxa-3-silahenicosan-21-yl 4-methylbenzenesulfonate (15.0 g, 27.2 mmol) at room temperature. The resulting mixture was stirred for overnight at 70° C. under nitrogen atmosphere. The mixture was allowed to cool to room temperature. The resulting mixture was diluted with EtOAc (200 mL). The organic layers were washed with water (2×50 mL) and brine (2×50 mL), dried over anhydrous MgSO₄. After filtration, the filtrate was concentrated under reduced pressure to afford the titled compound as a brown yellow oil. MS (ESI): m/z=581 [M+H]⁺.

### Step 2: N-methoxy-N-methyl-6-((2,2,3,3-tetramethyl-4,7,10,13,16,19-hexaoxa-3-silahenicosan-21-yl)oxy)-1-naphthamide

To a stirred mixture of methyl 6-[(2,2,3,3-tetramethyl-4,7,10,13,16,19-hexaoxa-3-silahenicosan-21-yl)oxy]naphthalene-1-carboxylate (15 g, 26 mmol) and N,O-dimethylhydroxylamine hydrochloride (3.78 g, 38.7 mmol) in THE (300 mL) were added i-PrMgCl (99 mL, 129 mmol) dropwise at 0° C. under argon atmosphere. The resulting mixture was stirred for 1 hour at 0° C. under argon atmosphere. The reaction was quenched by the addition of sat. NH₄Cl (aq.) (60 mL) at room temperature. The resulting mixture was extracted with EtOAc (3×60 mL). The combined organic layers were washed with brine (2×20 mL), dried over anhydrous Na₂SO₄. After filtration, the filtrate was concentrated under reduced pressure. The residue was purified by silica gel column chromatography, eluted with CH₂Cl₂/EA (1:1) to afford the titled compound (9.6 g, 52% yield) as a yellow oil. MS (ESI): m/z=610 [M+H]⁺.

### Step 3: 1-(6-((2,2,3,3-tetramethyl-4,7,10,13,16,19-hexaoxa-3-silahenicosan-21-yl)oxy) naphthalen-1-yl)ethan-1-one

To a stirred solution of N-methoxy-N-methyl-6-[(2,2,3,3-tetramethyl-4,7,10,13,16,19-hexaoxa-3-silahenicosan-21-yl)oxy]naphthalene-1-carboxamide (9.6 g, 16 mmol) in THF (38.4 mL) was added MeMgBr (157 mL, 157 mmol) at room temperature under argon atmosphere. The resulting mixture was stirred for 1 h at room temperature under argon atmosphere. The reaction was quenched by the addition of aqueous NH₄Cl (sat.) (50 mL) at room temperature. The resulting mixture was extracted with EtOAc (3×100 mL). The combined organic layers were washed with brine (2×60 mL), dried over anhydrous Na₂SO₄. After filtration, the filtrate was concentrated under reduced pressure. The residue was purified by silica gel column chromatography, eluted with CH₂Cl₂/EA (1:1) to afford the titled compound (6.5 g, 72% yield) as a light brown oil. ¹H NMR (300 MHz, DMSO-d₆) δ 8.54 (d, J=9.4 Hz, 1H), 8.05-7.92 (m, 2H), 7.53 (dd, J=8.2, 7.3 Hz, 1H), 7.41 (d, J=2.7 Hz, 1H), 7.26 (dd, J=9.4, 2.7 Hz, 1H), 4.26-4.16 (m, 2H), 3.84-3.75 (m, 2H), 3.68-3.36 (m, 20H), 2.68 (s, 3H), 0.82 (s, 9H). MS (ESI): m/z=565 (M+H)⁺.

### Step 4: 3-hydroxy-1-(6-((2,2,3,3-tetramethyl-4,7,10,13,16,19-hexaoxa-3-silahenicosan-21-yl)oxy)naphthalen-1-yl)butan-1-one

A solution of 1-(6-((2,2,3,3-tetramethyl-4,7,10,13,16,19-hexaoxa-3-silahenicosan-21-yl)oxy) naphthalen-1-yl)ethan-1-one (6.5 g, 11 mmol) in THF (260 mL) was treated with LiHMDS (15.9 mL, 0.796 mmol) for 10 min at −40° C. under nitrogen atmosphere followed by the addition of CH₃CHO (4.2 mL, 44 mmol) in THF dropwise in portions at −40° C., The resulting mixture was stirred for 30 min at −40° C. under nitrogen atmosphere. The reaction was quenched with aqueous NH₄Cl (sat.) at −40° C. The resulting mixture was extracted with EA (3×20 mL). The combined organic layers were washed with brine (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in H₂O, 10% to 95% gradient in 30 min; detector, UV 254 nm to afford the titled compound (1.65 g, 24% yield) as a yellow oil. ¹H NMR (400 MHz, DMSO-d₆) δ 8.37 (d, J=9.4 Hz, 1H), 7.99 (d, J=8.2 Hz, 1H), 7.88 (dd, J=7.2, 1.2 Hz, 1H), 7.55 (t, J=7.7 Hz, 1H), 7.43 (d, J=2.7 Hz, 1H), 7.27 (dd, J=9.4, 2.6 Hz, 1H), 4.72 (d, J=5.0 Hz, 1H), 4.23 (dd, J=5.7, 3.6 Hz, 2H), 4.23-4.11 (m, 1H), 3.85-3.79 (m, 2H), 3.74 (s, 1H), 3.70-3.59 (m, 4H), 3.63-3.53 (m, 2H), 3.57-3.47 (m, 13H), 3.43 (t, J=5.2 Hz, 2H), 1.16 (d, J=6.2 Hz, 3H), 0.85 (s, 9H). MS (ESI): m/z=609 [M+H]⁺.

### Step 5: 1-(6-((2,2,3,3-tetramethyl-4,7,10,13,16,19-hexaoxa-3-silahenicosan-21-yl)oxy)naphthalen-1-yl)butane-1,3-dione

A mixture of 3-hydroxy-1-(6-((2,2,3,3-tetramethyl-4,7,10,13,16,19-hexaoxa-3-silahenicosan-21-yl)oxy)naphthalen-1-yl)butan-1-one (1.5 g, 2.464 mmol) and Dess-Martin periodinane (1.36 g, 3.20 mmol) in CH₃C1 (20 mL) was stirred for 1 hour at room temperature under nitrogen atmosphere. The resulting mixture was filtered, the filter cake was washed with DCM (3×20 mL). The filtrate was concentrated under reduced pressure. The residue was purified by reversed phase flash chromatography with the following conditions: column, C18 silica gel; mobile phase, ACN in H₂O, 10% to 90% gradient in 30 min; detector, UV 254 nm to afford the title compound (1.1 g, 74% yield) as a yellow oil. ¹H NMR (400 MHz, DMSO-d₆) δ 8.34 (d, J=9.3 Hz, 1H), 8.00 (d, J=8.2 Hz, 1H), 7.65 (d, J=1.2 Hz, 1H), 7.59-7.52 (m, 1H), 7.45 (d, J=2.7 Hz, 1H), 7.28 (dd, J=9.4, 2.7 Hz, 1H), 4.29-4.16 (m, 2H), 3.83 (dd, J=3.8, 2.2 Hz, 2H), 3.67 (d, J=5.0 Hz, 2H), 3.62 (dd, J=4.6, 1.9 Hz, 2H), 3.58-3.54 (m, 2H), 3.53-3.49 (m, 14H), 3.42 (dd, J=6.0, 4.3 Hz, 2H), 2.21 (s, 3H), 0.85 (s, 12H). MS (ESI): n/z=607 [M+H]⁺.

### Step 6: (R)-(6-((17-hydroxy-3,6,9,12,15-pentaoxaheptadecyl)oxy)naphthalen-1-yl)(5-methyl-3-(morpholinomethyl)-2,3-dihydro-[1,4]oxazino[2,3,4-hi]indol-6-yl)methanone

A mixture of (3R)-3-(morpholin-4-ylmethyl)-2,3-dihydro-1,4-benzoxazin-4-amine (900 mg, 3.610 mmol), 1-(6-((2,2,3,3-tetramethyl-4,7,10,13,16,19-hexaoxa-3-silahenicosan-21-yl)oxy)naphthalen-1-yl)butane-1,3-dione (1.10 g, 1.81 mmol) in EtOH (18 mL) was stirred for 20 min at room temperature under nitrogen atmosphere, followed by the addition of H₂SO₄ (5% in EtOH, 3 mL) dropwise at room temperature. The resulting mixture was stirred for 2 hours at 78° C. under nitrogen atmosphere. The reaction was quenched by the addition of Na₂HCO₃ (sat) at 0° C. The resulting mixture was extracted with EA (3×30 mL). The combined organic layers were washed with brine (3×30 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in H₂O, 10% to 95% gradient in 30 min; detector, UV 254 nm to afford the titled compound (704.6 mg, 27% yield) as a yellow oil. ¹H NMR (300 MHz, DMSO-d₆) δ 7.98 (d, J=8.5 Hz, 1H), 7.72 (d, J=9.4 Hz, 1H), 7.57 (t, J=7.7 Hz, 1H), 7.47 (s, 1H), 7.39-7.30 (m, 1H), 7.15 (dd, J=9.2, 3.0 Hz, 1H), 6.85-6.73 (m, 1H), 6.64-6.55 (m, 1H), 6.32 (d, J=7.9 Hz, 1H), 4.88 (s, 1H), 4.73 (d, J=11.6 Hz, 1H), 4.59-4.56 (m, 1H), 4.27-4.13 (m, 3H), 3.82 (s, 2H), 3.67-3.50 (m, 22H), 3.48-3.36 (m, 2H), 2.61 (dd, J=12.7, 7.6 Hz, 1H), 2.49-2.18 (m, 8H). MS (ESI): m/z=707 [M+H]⁺.

### Step 6: (R)-(6-((17-azido-3,6,9,12,15-pentaoxaheptadecyl)oxy)naphthalen-1-yl)(5-methyl-3-(morpholinomethyl)-2,3-dihydro-[1,4]oxazino[2,3,4-hi]indol-6-yl)methanone

To a solution of (R)-(6-((17-hydroxy-3,6,9,12,15-pentaoxaheptadecyl)oxy)naphthalen-1-yl)(5-methyl-3-(morpholinomethyl)-2,3-dihydro-[1,4]oxazino[2,3,4-hi]indol-6-yl)methanone (0.25 g, 0.35 mmol) in anhydrous dichloromethane (3.5 mL), Et₃N (74 mL, 0.53 mmol) added followed by the MsCl (30 mL, 0.3 mmol) at 0° C. and slowly warmed up to room temperature. After 1 hour, the mixture was quenched with saturated NaHCO₃, extracted with dichloromethane (3×50 mL). Combined organic layer was washed with brine and dried over Na₂SO₄ filtered and concentrated to afford the crude product of mesylate which was carried to the next step without further purification. To a solution of the mesylate, prepared in the above step, in DMF (3.5 mL), NaN₃ (69 mg, 1.06 mmol) was added at room temperature and the reaction mixture was heated to 90° C. After 16 hours, the mixture was cooled to room temperature. The mixture was diluted with water, extracted with ethyl acetate (3×100 mL), combined organic layer was washed with brine, and dried over Na₂SO₄, filtered and concentrated. The residue was purified by flash chromatography (25 g, 20 micron Biotage column) using hexanes/EtOAc, 0-20% to afford the titled compound (232 mg, 67.4% yield) as a brown oil. 1H NMR (500 MHz, DMSO-d₆) δ 7.97 (d, J=10 Hz, 1H), 7.71 (d, J=5 Hz, 1H), 7.57-7.54 (m, 1H), 7.46 (d, J=5 Hz, 1H), 7.34-7.32 (m, 1H), 6.78 (t, J=10 Hz, 1H), 6.58 (d, J=10 Hz, 1H), 6.32 (d, J=10 Hz, 1H), 4.88-4.85 (br, 1H), 4.72 (d, J=10 Hz, 1H), 4.24-4.16 (m, 3H), 3.82-3.80 (m, H), 4.01-3.99 (m, 2H), 3.61-3.49 (m, 23H), 3.37-3.34 (m, 18H), 2.49-2.39 (m, 8H). MS (ESI): m/z=754.2 [M+Na]⁺.

BA-177 was conjugated to an oligo sense strand according to general procedure type I. The product (MW: 8443.26 g/mol) was made with 97% purity and confirmed with HPLC and LCMS (m/z: 8441.79, M).

BA-177 was bis-conjugated to an oligo sense strand according to general procedure type IIA. The product (MW: 9636.67 g/mol) was made with 86% purity and confirmed by HPLC and LCMS (m/z: 9634.71).

### Example 16: BA-156 Conjugate

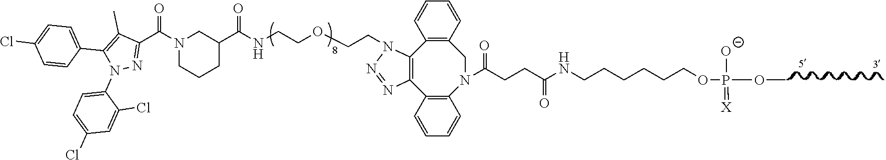

wherein X is O or S.

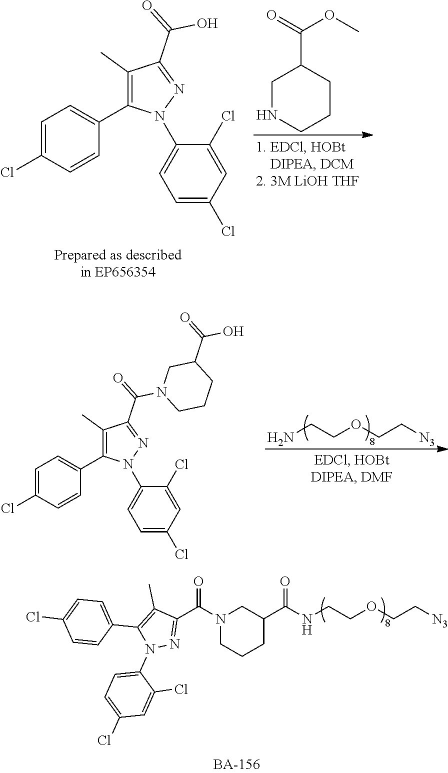

### Step 1: methyl 1-(5-(4-chlorophenyl)-1-(2,4-dichlorophenyl)-4-methyl-1H-pyrazole-3-carbonyl)piperidine-3-carboxylate

To a mixture of 5-(4-chlorophenyl)-1-(2,4-dichlorophenyl)-4-methyl-1H-pyrazole-3-carboxylic acid (0.5 g, 1.31 mmol), and methyl piperidine-3-carboxylate (0.206 g, 1.44 mmo) in DCM (5 mL) were successively added HOBt·H₂O (0.21 g, 1.3 mmol) and EDCI·HCl (0.33 g, 1.7 mmol). DIEA (0.3 mL, 1.70 mmol) was added dropwise, and the mixture was stirred at rt for 16 h. The mixture was concentrated then purified by silica-gel column chromatography using a gradient of ethyl acetate (0 to 100%) in hexanes to afford the titled compound (480 mg, 72% yield). MS (ESI) m/z=506.4 [M+H]⁺.

### Step 2: 1-(5-(4-chlorophenyl)-1-(2,4-dichlorophenyl)-4-methyl-1H-pyrazole-3-carbonyl)piperidine-3-carboxylic acid

To a solution of methyl 1-(5-(4-chlorophenyl)-1-(2,4-dichlorophenyl)-4-methyl-1H-pyrazole-3-carbonyl)piperidine-3-carboxylate (200 mg, 395 mol) in tetrahydrofuran (9.4 mL) was added 3M LiOH (0.16 mL, 0.474 mmol) dropwise at 0° C. The cooling bath was removed, and the mixture was stirred overnight at rt. Another portion of 3M LiOH (0.16 mL, 0.474 mmol) was added, and the mixture was stirred for 3 hours at room temperature then concentrated. The residue obtained was purified by reverse-phase column chromatography using a gradient 0-100% acetonitrile in water (+0.1% formic acid) to afford the titled compound (174 mg, 90% yield). MS (ESI): m/z=494.4 [M+H]⁺.

### Step 3: N-(26-azido-3,6,9,12,15,18,21,24-octaoxahexacosyl)-1-(5-(4-chlorophenyl)-1-(2,4-dichlorophenyl)-4-methyl-1H-pyrazole-3-carbonyl)piperidine-3-carboxamide

To a solution of 1-(5-(4-chlorophenyl)-1-(2,4-dichlorophenyl)-4-methyl-1H-pyrazole-3-carbonyl) piperidine-3-carboxylic acid (0.1 g, 0.2 mmol), DIEA (0.053 mL, 0.304 mmol) and HOBt (0.036 g, 0.264 mmol) in DMF (1 mL), was added EDCI·HCl (0.058 g, 0.304 mmol) and the mixture was stirred for 4 hours at room temperature. The mixture was concentrated, and the residue was purified by reverse-phase column chromatography using a gradient 0-50% acetonitrile in water (+0.1% formic acid) to afford the titled compound (98 mg, 53% yield). MS (ESI): m/z=914.4 [M+H]⁺.

BA-156 was conjugated to an oligo sense strand according to general procedure type 1. The product (MW: 8213.36 g/mol) was made with 97% purity and confirmed by HPLC and LCMS (m/z: 8211.91).

### Example 17: BA-164 Conjugate

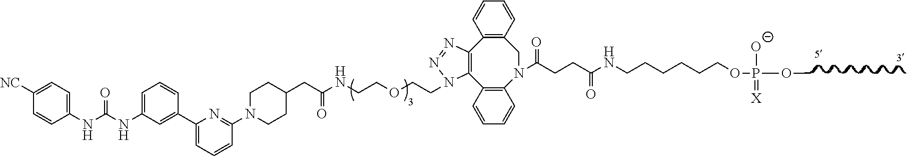

wherein X is O or S.

### BA-164: N-(2-(2-(2-(2-azidoethoxy)ethoxy)ethoxy)ethyl)-2-(1-(6-(3-(3-(4-cyanophenyl)ureido)phenyl)pyridin-2-yl)piperidin-4-yl)acetamide

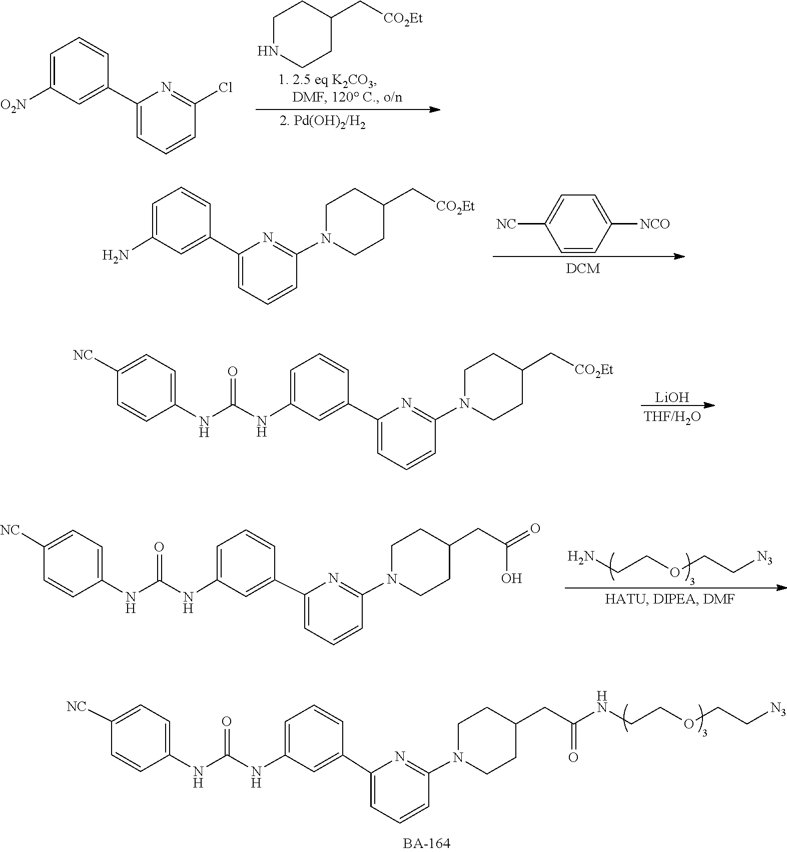

### Step 1: ethyl 2-(1-(6-(3-nitrophenyl)pyridin-2-yl)piperidin-4-yl)acetate

Combined 2-chloro-6-(3-nitrophenyl)pyridine (2.5 g, 11 mmol) and ethyl 2-(piperidin-4-yl)acetate (5.5 g, 32 mmol) in DMF (10 mL), and heated the mixture at 105° C. for overnight. After cooling, the mixture was treated with water (10 mL) and extracted with DCM (2×100 mL). The combined organic layers were dried over Na₂SO₄, filtered and concentrated. The residue was purified by reversed-phase flash chromatography with the following conditions: column, C18 silica gel; mobile phase, MeCN in Water, 30% to 95% gradient in 25 min; detector, UV 254 nm, to afford the titled compound (2.4 g, 61% yield) as a yellow oil. 1H NMR (300 MHz, DMSO-d₆) δ 8.80 (t, J=2.0 Hz, 1H), 8.48 (dt, J=7.9, 1.4 Hz, 1H), 8.24 (ddd, J=8.2, 2.4, 1.0 Hz, 1H), 7.81-7.60 (m, 2H), 7.33 (d, J=7.4 Hz, 1H), 6.90 (d, J=8.5 Hz, 1H), 4.42 (d, J=12.6 Hz, 2H), 4.08 (q, J=7.1 Hz, 2H), 2.97-2.82 (m, 2H), 2.31-2.13 (m, 2H), 1.97 (ddt, J=11.1, 7.4, 3.7 Hz, 1H), 1.82-1.70 (m, 2H), 1.32-1.12 (m, 5H). MS (ESI) m/z=370.0 [M+H]⁺.

### Step 2: ethyl 2-(1-(6-(3-aminophenyl)pyridin-2-yl)piperidin-4-yl)acetate

A mixture of ethyl 2-(1-(6-(3-nitrophenyl)pyridin-2-yl)piperidin-4-yl)acetate (2.2 g, 5.9 mmol) and Pd(OH)₂ on carbon (0.66 g, 4.7 mmol) in MeOH (44 mL) was stirred for overnight at room temperature under hydrogen atmosphere. The resulting mixture was filtered, the filter cake was washed with MeOH. The filtrate was concentrated under reduced pressure to afford the titled compound (2 g, 98% yield) as a yellow oil. ¹H NMR (300 MHz, DMSO-d₆) δ 7.55 (dd, J=8.5, 7.4 Hz, 1H), 7.26 (t, J=1.9 Hz, 1H), 7.14 (dt, J=7.7, 1.5 Hz, 1H), 7.13-6.98 (m, 2H), 6.74 (d, J=8.5 Hz, 1H), 6.58 (ddd, J=7.6, 2.4, 1.3 Hz, 1H), 5.12 (s, 2H), 4.40 (d, J=13.0 Hz, 2H), 4.08 (q, J=7.1 Hz, 2H), 3.18 (s, 2H), 2.91-2.68 (m, 2H), 2.27 (d, J=7.0 Hz, 2H), 1.96 (tt, J=7.3, 3.7 Hz, OH), 1.75 (d, J=12.3 Hz, 2H), 1.32-1.09 (m, 6H). MS (ESI) m/z=340.2 [M+H]⁺.

### Step 3: ethyl 2-(1-(6-(3-(3-(4-cyanophenyl)ureido)phenyl)pyridin-2-yl)piperidin-4-yl)acetate

A mixture of ethyl 2-(1-(6-(3-aminophenyl)pyridin-2-yl)piperidin-4-yl)acetate (2.0 g, 5.9 mmol) and 4-isocyanatobenzonitrile (0.85 g, 5.892 mmol) in DCM (10 mL) was stirred for overnight at room temperature under argon atmosphere. The resulting mixture was concentrated under reduced pressure to afford the titled compound (3.11 g, 100% yield) as a white solid. The product was used in the next step directly without further purification. 1H NMR (300 MHz, DMSO-d₆) δ 9.26 (s, 1H), 9.00 (s, 1H), 8.04 (d, J=1.9 Hz, 1H), 7.73 (d, J=8.8 Hz, 2H), 7.69-7.53 (m, 5H), 7.36 (t, J=7.9 Hz, 1H), 7.10 (d, J=7.4 Hz, 1H), 6.79 (d, J=8.5 Hz, 1H), 5.75 (s, OH), 4.40 (d, J=13.0 Hz, 2H), 4.06 (q, J=7.1 Hz, 2H), 2.85 (t, J=12.4 Hz, 2H), 2.26 (d, J=7.0 Hz, 2H), 1.95 (s, 1H), 1.73 (d, J=12.9 Hz, 2H), 1.23 (s, 1H), 1.17 (t, J=7.1 Hz, 4H). MS (ESI) m/z=484.2 [M+H]⁺.

### Step 4: 2-(1-(6-(3-(3-(4-cyanophenyl)ureido)phenyl)pyridin-2-yl)piperidin-4-yl)acetic acid

A mixture of ethyl 2-(1-(6-(3-(3-(4-cyanophenyl)ureido)phenyl)pyridin-2-yl)piperidin-4-yl)acetate (2.66 g, 5.50 mmol) and LiOH (0.53 g, 22.0 mmol) in THF (26.6 mL, 328.318 mmol) and H₂O (27 mL) was stirred for overnight at room temperature. The mixture was acidified to pH 5 with acetic acid (1.98 g, 33 mmol). The reaction was quenched with water (2 mL) at room temperature. The resulting mixture was extracted with ethyl acetate (100 mL). The combined organic layers were washed with brine (20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water, 20% to 90% gradient in 30 min; detector, UV 254 nm to afford the titled compound (1.92 g, 76% yield) as a light-yellow solid. 1H NMR (300 MHz, DMSO-d₆) δ 12.10 (s, 1H), 9.25 (s, 1H), 9.00 (s, 1H), 8.06 (t, J=2.0 Hz, 1H), 7.75 (d, J=8.8 Hz, 2H), 7.76-7.57 (m, 4H), 7.59 (s, 1H), 7.38 (t, J=7.9 Hz, 1H), 7.12 (d, J=7.4 Hz, 1H), 6.81 (d, J=8.5 Hz, 1H), 4.42 (d, J=12.9 Hz, 2H), 2.87 (t, J=12.3 Hz, 2H), 2.20 (d, J=6.9 Hz, 2H), 2.08 (s, 1H), 1.95 (s, 1H), 1.78 (d, J=12.3 Hz, 2H), 1.22 (td, J=14.8, 14.1, 6.8 Hz, 2H). MS (ESI) m/z=456.2 [M+H]⁺.

### Step 5: N-(2-(2-(2-(2-azidoethoxy)ethoxy)ethoxy)ethyl)-2-(1-(6-(3-(3-(4-cyanophenyl)ureido) phenyl)pyridin-2-yl)piperidin-4-yl)acetamide

A solution of the 2-(1-(6-(3-(3-(4-cyanophenyl)ureido)phenyl)pyridin-2-yl)piperidin-4-yl)acetic acid (0.2 g, 0.44 mmol), DIPEA (0.15 mL, 0.9 mmol) and 2-(2-(2-(2-azidoethoxy)ethoxy)ethoxy)-ethan-1-amine (0.12 g, 0.55 mmol) in DMA (3 mL) was treated with HATU (0.34 g, 0.88 mmol) and stirred for 3 hours. The reaction mixture was added slowly to a mixture of water (150 mL) and saturated NH₄Cl (10 mL, aq.) to form a precipitate. The solids were filtered, washed with water, and air-dried. The solid was redissolved in DCM, dried over Na₂SO₄, filtered and concentrate. The residue was purified by silica gel chromatography (0 to 50% methanol in ethyl acetate, 10 g Biotage column) to afford the titled compound (221 mg, 77% yield) as a yellow oil. ¹H NMR (499 MHz, DMSO-d₆) δ 9.23 (s, 1H), 8.97 (s, 1H), 8.05 (t, J=2.0 Hz, 1H), 7.87 (t, J=5.7 Hz, 1H), 7.77-7.70 (m, 2H), 7.69-7.54 (m, 5H), 7.37 (t, J=7.9 Hz, 1H), 7.11 (d, J=7.4 Hz, 1H), 6.79 (d, J=8.5 Hz, 1H), 4.43-4.36 (m, 2H), 3.62-3.46 (m, 10H), 3.39 (dt, J=14.7, 5.4 Hz, 4H), 3.20 (q, J=5.8 Hz, 2H), 2.91-2.80 (m, 2H), 2.01 (d, J=21.6 Hz, 4H), 1.75-1.66 (m, 2H), 1.24-1.11 (m, 4H). MS (ESI) m/z=656.6 for [M+H]⁺, 678.5 for [M+Na]⁺.

BA-164 was conjugated to an oligo sense strand according to general procedure type I. The product (MW: 8367.17 g/mol) was made with 98% purity and confirmed by HPLC and LCMS (m/z: 8365.37).

### Example 18: BA-165 Conjugate

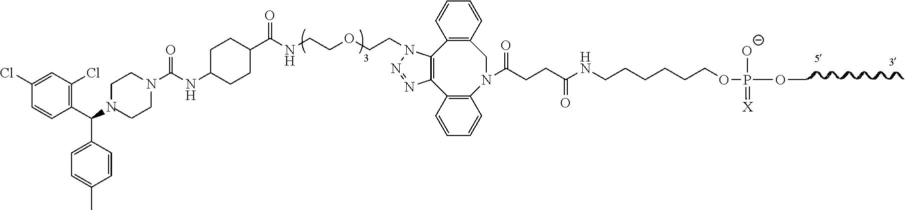

wherein X is O or S.

### BA-165 (S)—N-(4-((2-(2-(2-(2-azidoethoxy)ethoxy)ethoxy)ethyl)carbamoyl)cyclohexyl)-4-((2,4-dichlorophenyl)(p-tolyl)methyl)piperazine-1-carboxamide

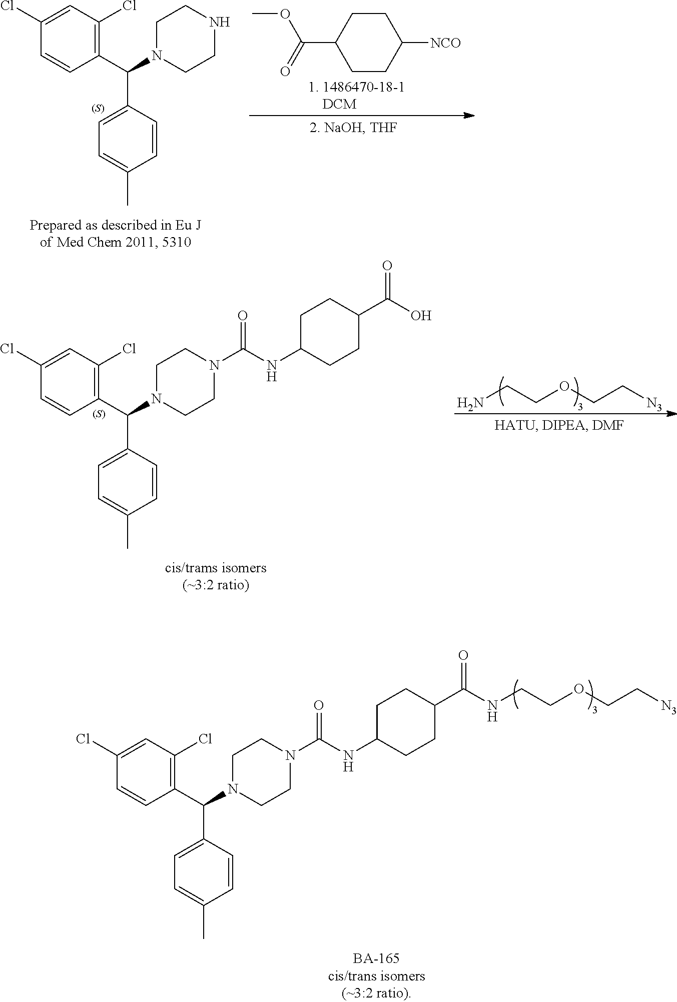

### Step 1: (S)-4-(4-((2,4-dichlorophenyl)(p-tolyl)methyl)piperazine-1-carboxamido)cyclohexane-1-carboxylic acid

A solution of methyl 4-isocyanatocyclohexane-1-carboxylate (3.06 g, 4.2 mmol) in DCM (28.00 mL) was treated with 1-[(S)-(2,4-dichlorophenyl)(4-methylphenyl)methyl]piperazine (1.4 g, 4.2 mmol) at room temperature under nitrogen atmosphere and stirred for 3 hours. The resulting mixture was concentrated under reduced pressure. The residue was purified by reversed-phase flash chromatography with the following conditions: column, C18 silica gel; mobile phase, MeCN in water, 40% to 100% gradient in 10 min; detector, UV 254 nm to afford the methyl ester (2.0 g, 92% yield) as an off-white solid. To a stirred solution of methyl ester prepared (2.0 g, 3.9 mmol) in THF (50 mL) was added 1N NaOH (19 mL, 19 mmol) dropwise at room temperature. The resulting mixture was stirred for overnight at room temperature. The mixture was acidified to pH 5 with AcOH. The mixture was extracted with ethyl acetate (100 mL×2). The combined organic layer was dried over Na₂SO₄, filtered and concentrated. The residue was purified by reversed-phase flash chromatography with the following conditions: column, C18 silica gel; mobile phase, MeCN in Water, 10% to 100% gradient in 10 min; detector, UV 220 nm to afford the titled compound (1.7 g, 87% yield) as an off-white solid. It is a mixture of cis/trans isomers. 1H NMR (400 MHz, DMSO-d₆) δ 12.09 (s, 111), 7.83 (d, J=8.4 Hz, 111), 7.52 (q, J=2.2 Hz, 1H), 7.46 (dt, J=8.5, 2.5 Hz, 1H), 7.24 (d, J=7.8 Hz, 2H), 7.11 (dd, J=8.3, 2.3 Hz, 2H), 6.12 (dd, J=20.0, 7.4 Hz, 1H), 4.63 (s, 1H), 3.46 (s, 1H), 3.27 (d, J=5.1 Hz, 4H), 2.43-2.01 (m, 8H), 1.96-1.69 (m, 3H), 1.35 (m, 5H).

### Step 2: (S)—N-(4-((2-(2-(2-(2-azidoethoxy)ethoxy)ethoxy)ethyl)carbamoyl)cyclohexyl)-4-((2,4-dichlorophenyl)(p-tolyl)methyl)piperazine-1-carboxamide

A solution of the (S)-4-(4-((2,4-dichlorophenyl)(p-tolyl)methyl)piperazine-1-carboxamido)cyclo hexane-1-carboxylic acid (mixture of cis/trans isomers; 0.2 g, 0.4 mmol), DIPEA (0.14 mL, 0.8 mmol) and 2-(2-(2-(2-azidoethoxy)ethoxy)ethoxy)ethan-1-amine (0.11 g, 0.5 mmol) in DMA (3 mL) was treated with HATU (0.23 g, 0.6 mmol) and stirred for 3 hours. The reaction mixture was added slowly to a mixture of water (50 mL) and sat. NH₄Cl (5 mL), the precipitated solid was filtered, washed with water, and air-dried. The crude product was redissolved in DCM, dried with Na₂SO₄, filtered and concentrated. The residue was purified by silica gel chromatography (0 to 50% MeOH in ethyl acetate, 12 g Claricep column) to afford the titled compound (194 mg, 69% yield) as a yellow oil and a mixture of cis/trans isomers (˜3:2 ratio). 1H NMR (499 MHz, DMSO-d₆) δ 7.83 (d, J=8.5 Hz, 1H), 7.73 (t, J=5.7 Hz, 1H), 7.51 (d, J=2.1 Hz, 1H), 7.46 (dd, J=8.5, 2.2 Hz, 1H), 7.24 (d, J=7.9 Hz, 2H), 7.11 (d, J=7.8 Hz, 2H), 6.11 (d, J=7.8 Hz, 1H), 4.63 (s, 1H), 3.59 (td, J=4.9, 2.3 Hz, 2H), 3.56-3.45 (m, 9H), 3.38 (td, J=6.0, 3.3 Hz, 5H), 3.31-3.22 (m, 5H), 3.17 (d, J=6.1 Hz, 2H), 2.21 (ddd, J=28.9, 14.1, 5.2 Hz, 4H), 2.01 (tt, J=12.0, 3.3 Hz, 1H), 1.83-1.73 (m, 3H), 1.73-1.64 (m, 2H), 1.57 (q, J=8.0, 6.5 Hz, 1H), 1.51-1.39 (m, 2H), 1.34 (td, J=12.7, 3.3 Hz, 1H), 1.21-1.09 (m, 1H). MS (ESI): m/z 704.7 for [M+H]⁺, =728.6 for [M+Na]⁺.

BA-165 was conjugated to an oligo sense strand according to general procedure type I. The product (MW: 8416.10 g/mol) was made with 98% purity and confirmed by HPLC and LCMS (m/z: 8414.31).

### Example 19: BA-168 Conjugates

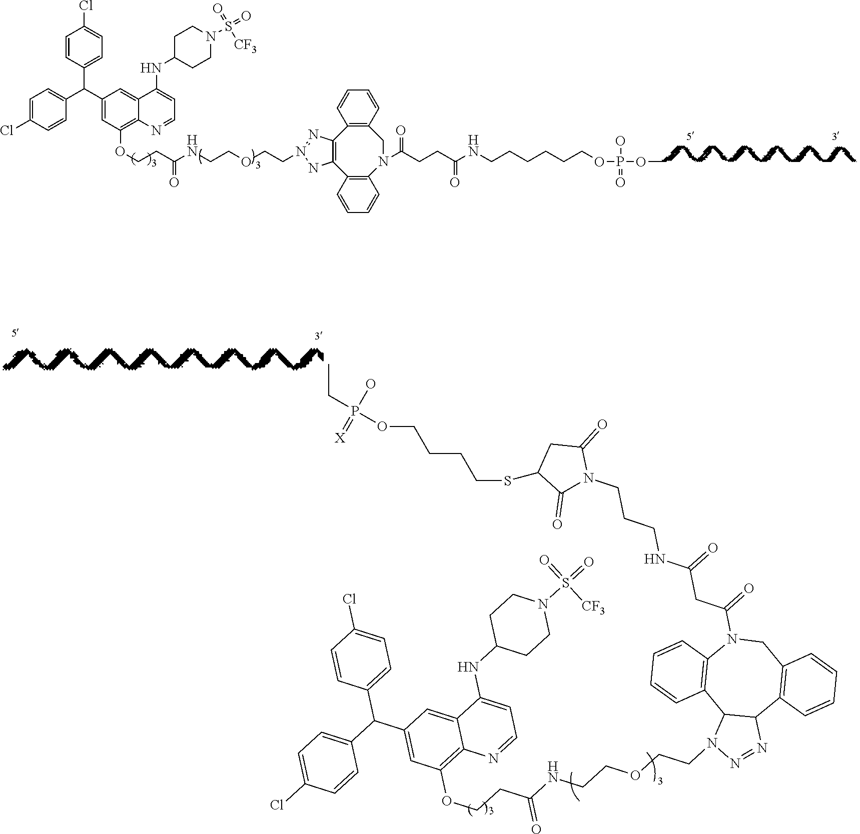

wherein X is O or S.

### BA-168: N-(2-(2-(2-(2-azidoethoxy)ethoxy)ethoxy)ethyl)-5-((6-(bis(4-chlorophenyl)methyl)-4-((1-((trifluoromethyl)sulfonyl)piperidin-4-yl)amino)quinolin-8-yl)oxy)pentanamide

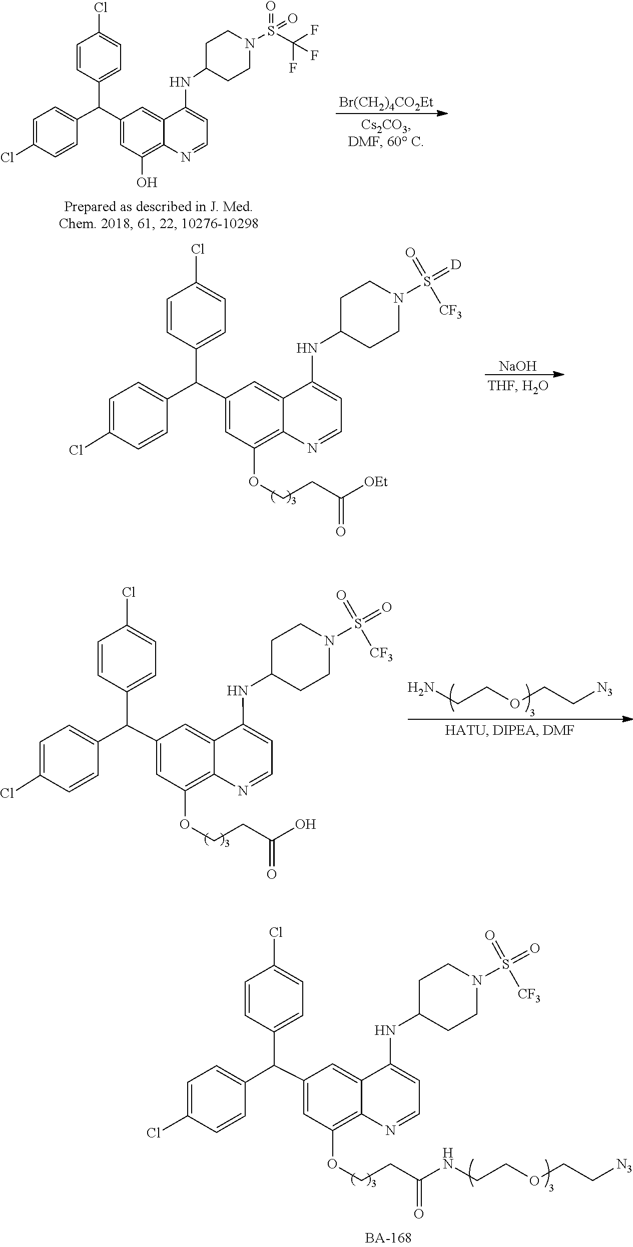

### Step 1: Ethyl 5-((6-(bis(4-chlorophenyl)methyl)-4-((1-((trifluoromethyl)sulfonyl)piperidin-4-yl)amino)quinolin-8-yl)oxy)pentanoate

To a mixture of 6-[bis(4-chlorophenyl)methyl]-4-[(1-trifluoromethanesulfonyl piperidin-4-yl)amino]quinolin-8-ol (2.0 g, 3.3 mmol, prepared as described in J Med Chem 2018, 10276) and ethyl 5-bromopentanoate (0.75 g, 3.6 mmol) in DMF (5 mL) were added Cs₂CO₃ (3.20 g, 9.8 mmol) at room temperature under nitrogen atmosphere. The resulting mixture was stirred for overnight at 60° C. The resulting mixture was diluted with ethyl acetate (100 mL) and was washed with water (3×100 mL). The organic layer was dried Na₂SO₄, filtered and concentrated. The residue was purified by silica gel column chromatography, eluted with DCM:MeOH (10:1, V/V) to afford the titled compound (1.9 g, 78% yield) as a yellow solid. ¹H NMR (400 MHz, DMSO-d₆) δ 8.37 (d, J=5.9 Hz, 1H), 7.82 (s, 1H), 7.57-7.47 (m, 1H), 7.45-7.32 (m, 4H), 7.31 (s, OH), 7.25-7.17 (m, 4H), 6.93 (s, 1H), 6.76 (d, J=5.9 Hz, 1H), 5.71 (s, 1H), 4.08-3.98 (m, 4H), 3.98-3.86 (m, 4H), 3.40 (t, J=12.7 Hz, 2H), 2.36 (t, J=7.2 Hz, 2H), 2.10 (d, J=12.7 Hz, 2H), 1.74 (s, 4H), 1.71 (s, 1H), 1.72-1.65 (m, 2H), 1.24 (s, 1H), 1.16 (t, J=7.1 Hz, 3H), MS (ESI): m/z=738.2 [M+H]⁺.

### Step 2: 5-((6-(bis(4-chlorophenyl)methyl)-4-((1-((trifluoromethyl)sulfonyl)piperidin-4-yl)amino)quinolin-8-yl)oxy)pentanoic acid

Add 1M NaOH (aq, 7.8 mL, 25 mmol) to a mixture of (ethyl 5-((6-(bis(4-chlorophenyl)methyl)-4-((1-((trifluoromethyl)sulfonyl)piperidin-4-yl)amino)quinolin-8-yl)oxy)pentanoate (1.9 g, 2.6 mmol) in THF (15 mL), was stirred for overnight at room temperature under nitrogen atmosphere. The reaction mixture was neutralized and quenched with glacial acetic acid. The resulting mixture was extracted with ethyl acetate (3×100 mL). The combined organic layers were washed with brine (3×100 mL), dried over anhydrous Na₂SO₄, filtered and concentrated. The residue was purified by reversed-phase flash chromatography with the following conditions: column, C18 silica gel; mobile phase, MeCN in water, 10% to 100% gradient in 20 min; detector, UV 254 nm. to obtain the titled compound (484 mg, 26% yield) as a light yellow solid. ¹H NMR (400 MHz, DMSO-d₆) δ 8.35 (d, J=5.3 Hz, 1H), 7.73 (s, 1H), 7.40 (d, J=8.2 Hz, 4H), 7.21 (d, J=8.2 Hz, 4H), 6.78 (d, J=10.1 Hz, 2H), 6.65 (d, J=5.6 Hz, 1H), 5.77 (s, 1H), 5.68 (s, 1H), 3.98 (t, J=5.8 Hz, 2H), 3.88 (d, J=13.4 Hz, 3H), 3.40 (t, J=12.4 Hz, 2H), 2.31 (t, J=7.0 Hz, 2H), 2.15-2.06 (m, 2H), 1.79-1.57 (m, 6H). MS (ESI): n/z=710.0 [M+H]⁺.

### Step 3: N-(2-(2-(2-(2-azidoethoxy)ethoxy)ethoxy)ethyl)-5-((6-(bis(4-chlorophenyl)methyl)-4-((1-((trifluoromethyl)sulfonyl)piperidin-4-yl)amino)quinolin-8-yl)oxy)pentanamide

A solution of the acid, 5-((6-(bis(4-chlorophenyl)methyl)-4-((1-((trifluoromethyl)sulfonyl) piperidin-4-yl)amino)quinolin-8-yl)oxy)pentanoic acid (0.20 g, 0.44 mmol), DIPEA (0.15 mL, 0.90 mmol) and amine (0.12 g, 0.55 mmol, 1.25 equiv.) in DMA (3 mL) was treated with HATU (0.34 g, 0.88 mmol) and stirred for 3 hours. The reaction mixture was diluted with ethyl acetate (50 mL) and washed with water (2×100 mL). The organic layer was washed with sat. NaHCO₃ (50 mL), brine (50 mL), dried over Na₂SO₄, filtered and concentrated. The residue was purified and by silica gel chromatography (0 to 100% ethyl acetate in hexane, followed by 0 to 40% methanol in ethyl acetate, 12 g Claricep column) to afford the titled compound (231 mg, 90% yield) as a clear oil. ¹H NMR (499 MHz, DMSO-d₆) δ 8.45 (s, 1H), 8.38 (d, J=5.9 Hz, 1H), 7.82 (s, 1H), 7.48-7.29 (m, 4H), 7.25-7.17 (m, 4H), 6.92 (s, 1H), 6.78 (d, J=6.0 Hz, 1H), 5.73 (d, J=20.1 Hz, 2H), 4.07-3.85 (m, 6H), 3.59-3.53 (m, 2H), 3.53-3.45 (m, 4H), 3.38 (dt, J=21.8, 5.4 Hz, 5H), 3.22 (q, J=5.9 Hz, 2H), 2.19 (t, J=6.8 Hz, 2H), 2.10 (dd, J=13.5, 3.8 Hz, 2H), 1.70 (tt, J=12.7, 7.9 Hz, 7H). MS (ESI): m/z=910.6 [M+H]⁺.

BA-168 was conjugated to an oligo sense strand according to general procedure type I. The product (MW: 8620.51 g/mol) was made with 92% purity and confirmed by HPLC and LCMS (m/z: 8622.24).

### Example 20: BA-120 Conjugates

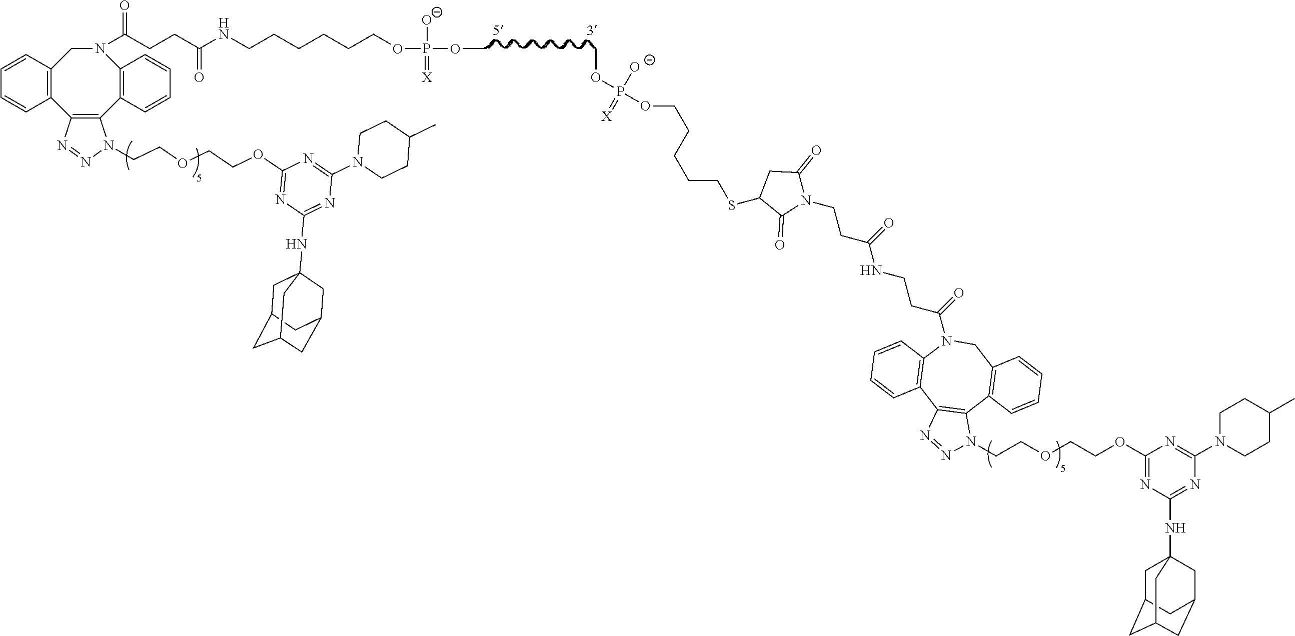

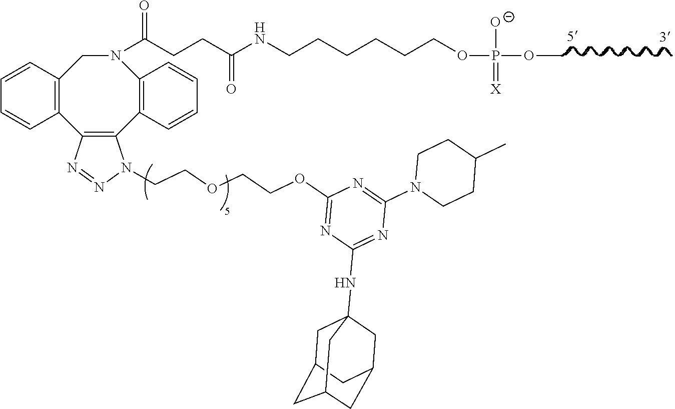

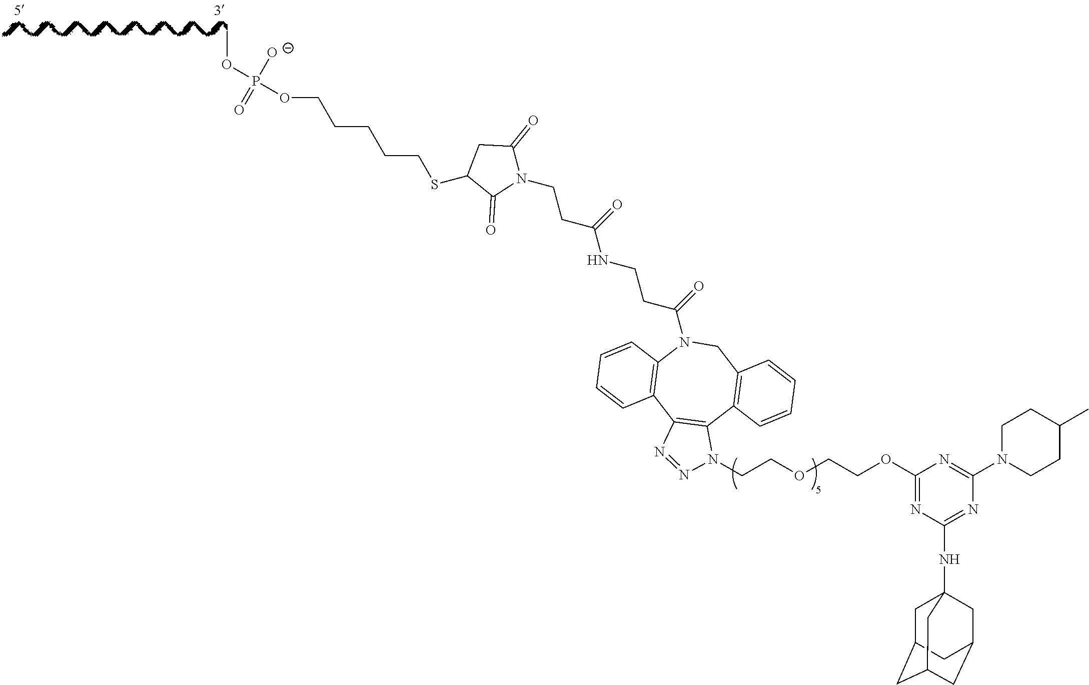

wherein X is O or S.

### BA-120 N-((3s,5s,7s)-adamantan-1-yl)-4-((17-azido-3,6,9,12,15-pentaoxaheptadecyl)oxy)-6-(4-methylpiperidin-1-yl)-1,3,5-triazin-2-amine

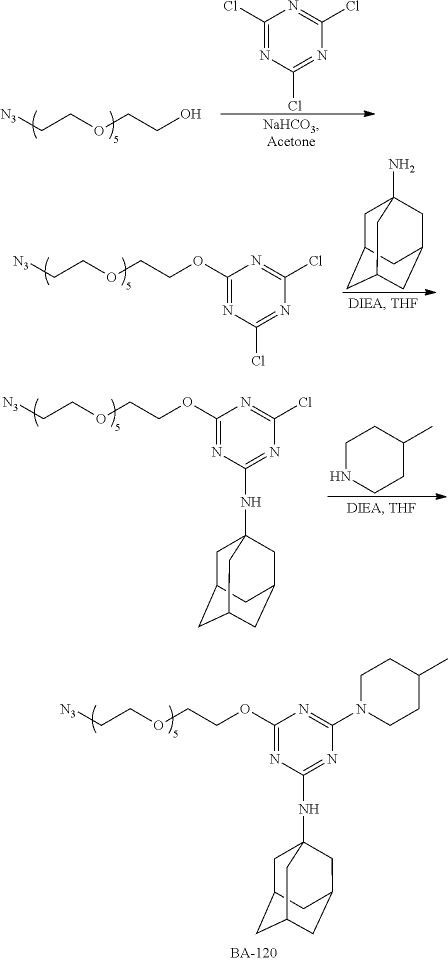

### Step 1: 2-((17-azido-3,6,9,12,15-pentaoxaheptadecyl)oxy)-4,6-dichloro-1,3,5-triazine

To a suspension of 2,4,6-trichloro-1,3,5-triazine (1.0 g, 5.42 mmol) and NaHCO₃ (911 mg, 10.84 mmol) in acetone (8 mL) at 0° C. was added 17-azido-3,6,9,12,15-pentaoxaheptadecan-1-ol (1.67 g, 5.42 mmol) over 10 mins. The cooling bath was removed, and the mixture was stirred for 20 hours at room temperature. The solvent was evaporated, and the residue obtained was partitioned between water and DCM. An emulsion formed. Brine was added and the organic phase was separated. The organic phase was washed with brine, dried, filtered, concentrated and the residue obtained was purified by silica-gel column chromatography using a gradient 0-100% ethyl acetate in hexanes to afford the titled compound (1.48 g, 60% yield). MS (ESI): m/z=455.3 [M+H]⁺.

### Step 2: N-((3s,5s,7s)-adamantan-1-yl)-4-((17-azido-3,6,9,12,15-pentaoxaheptadecyl)oxy)-6-chloro-1,3,5-triazin-2-amine

To a mixture of 2-((17-azido-3,6,9,12,15-pentaoxaheptadecyl)oxy)-4,6-dichloro-1,3,5-triazine (500 mg, 1.1 mmol) in THF (9.5 mL) at 0° C. was added a mixture of adamantylamine (166 mg, 0.151 ml, 1.1 mmol) and DIEA (0.29 mL, 1.65 mmol) in THF (1.2 mL) dropwise. The cooling bath was removed, and the mixture was stirred overnight at rt then concentrated. The residue obtained was purified by silica-gel column chromatography using a gradient 0-85% ethyl acetate in hexanes to afford the titled compound (282 mg, 90% yield). MS (ESI)/z=570.6 [M+H]⁺.

### Step 3: N-((3s,5s,7s)-adamantan-1-yl)-4-((17-azido-3,6,9,12,15-pentaoxaheptadecyl)oxy)-6-(4-methylpiperidin-1-yl)-1,3,5-triazin-2-amine

To a N-((3s,5s,7s)-adamantan-1-yl)-4-((17-azido-3,6,9,12,15-pentaoxaheptadecyl)oxy)-6-chloro-1,3,5-triazin-2-amine (50 mg, 0.088 mmol) in anhydrous THF (0.76 ml) at 0° C. was added a mixture of 4-methylpiperidine (16 mL, 0.13 mmol) and DIEA (18 mL, 0.10 mmol) in THF (0.1 mL) dropwise. The mixture was stirred at 60° C. for 3 hours then concentrated and the residue was purified by reverse-phase column chromatography using a gradient 0-100% acetonitrile in water (+0.1% formic acid) to afford the tilted compound in 65% (36 mg, 65% yield). MS (EST): m/z 633.2 [M+H]⁺, 655.2 [M+Na]⁺.

BA-120 was conjugated to an oligo sense strand according to general procedure type I. The product (MW: 8344.22 g/mol) was made with 86% purity and confirmed by HPLC and LCMS (m/z: 8342.68).

BA-120 was conjugated to an oligo sense strand according to general procedure type I. The product (MW: 8305.13 g/mol) was made with 98% purity and confirmed by HPLC and LCMS (m/z: 8304.13).

BA-120 was conjugated to an oligo sense strand according to general procedure type I. The product (MW: 7932.88 g/mol) was made with 92% purity and confirmed by HPLC and LCMS (m/z: 7931.34).

BA-120 was bis-conjugated to an oligo sense strand according to general procedure type IIA. The product (MW: 9420.57 g/mol) was made with 92% purity was confirmed by HPLC and LCMS (m/z: 9418.87)

### Example 21: BA-146 Conjugate

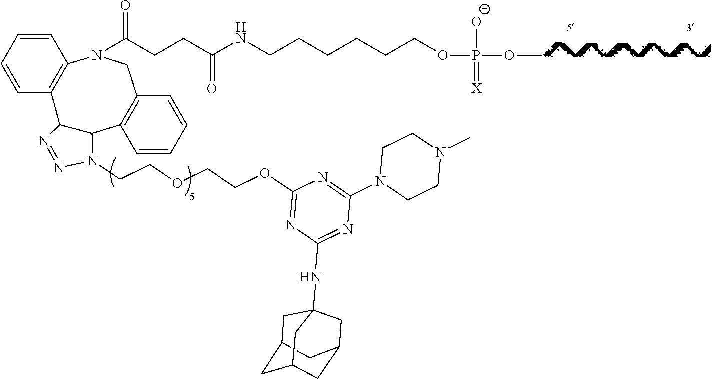

wherein X is O or S.

### BA-146: N-((3s,5s,7s)-adamantan-1-yl)-4-((17-azido-3,6,9,12,15-pentaoxaheptadecyl)oxy)-6-(4-methylpiperazin-1-yl)-1,3,5-triazin-2-amine formate

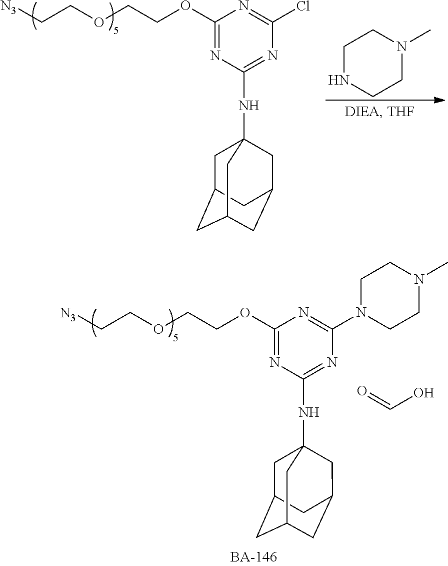

The compound is prepared using the same procedure as BA-120 by replacing the 4-methylpiperidine with 1-methylpiperazine to afford the titled compound as a yellow oil. MS (ESI): m/z=634.2 [M+H]⁺.

BA-146 was conjugated to an oligo sense strand according to general procedure type I. The product (MW: 8345.21 g/mol) was made with 99% purity and confirmed by HPLC and LCMS (m/z: 8342.75).

### Example 22: BA-147 Conjugate

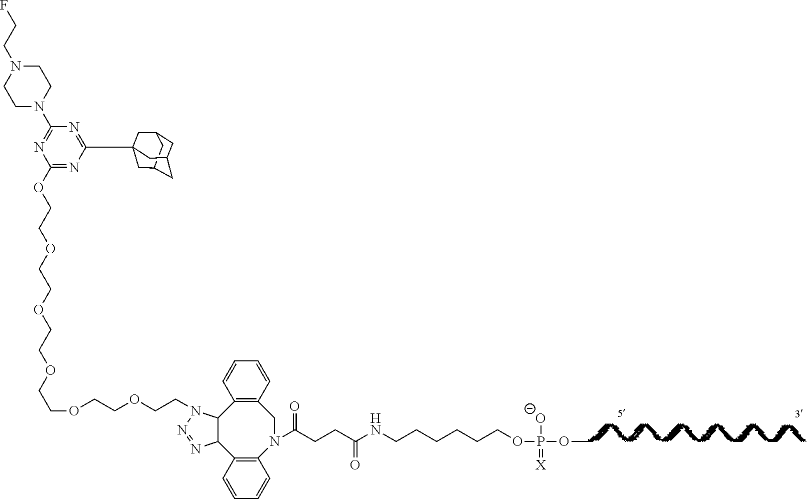

wherein X is O or S.

### BA-147: N-((3s,5s,7s)-adamantan-1-yl)-4-((17-azido-3,6,9,12,15-pentaoxaheptadecyl)oxy)-6-(4-(2-fluoroethyl)piperazin-1-yl)-1,3,5-triazin-2-amine formate

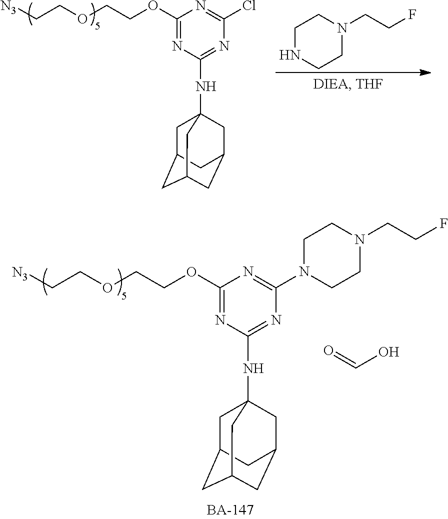

The compound is prepared using the same procedure as BA-120 by replacing the 4-methylpiperidine with fluoroethylpiperazine to afford the titled compound as a yellow oil. MS (ESI): m/z=666.4 [M+H]⁺.

BA-147 was conjugated to an oligo sense strand according to general procedure type I. The product (MW: 7965.88.40 g/mol) was made with 88% purity and confirmed by HPLC and LCMS (m/z: 7964.63).

### Example 23: BA-152 Conjugate

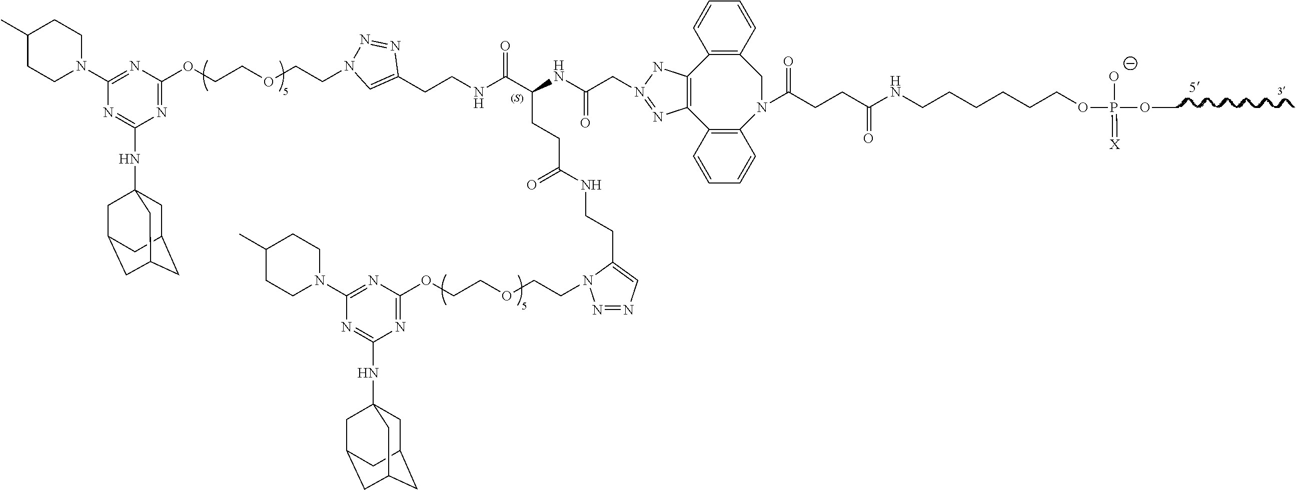

wherein X is O or S.

### BA-152: (S)—N1,N5-bis(2-(1-(17-((4-(adamantan-1-ylamino)-6-(4-methylpiperidin-1-yl)-1,3,5-triazin-2-yl)oxy)-3,6,9,12,15-pentaoxaheptadecyl)-1H-1,2,3-triazol-4-yl)ethyl)-2-(2-azidoacetamido)pentanediamide

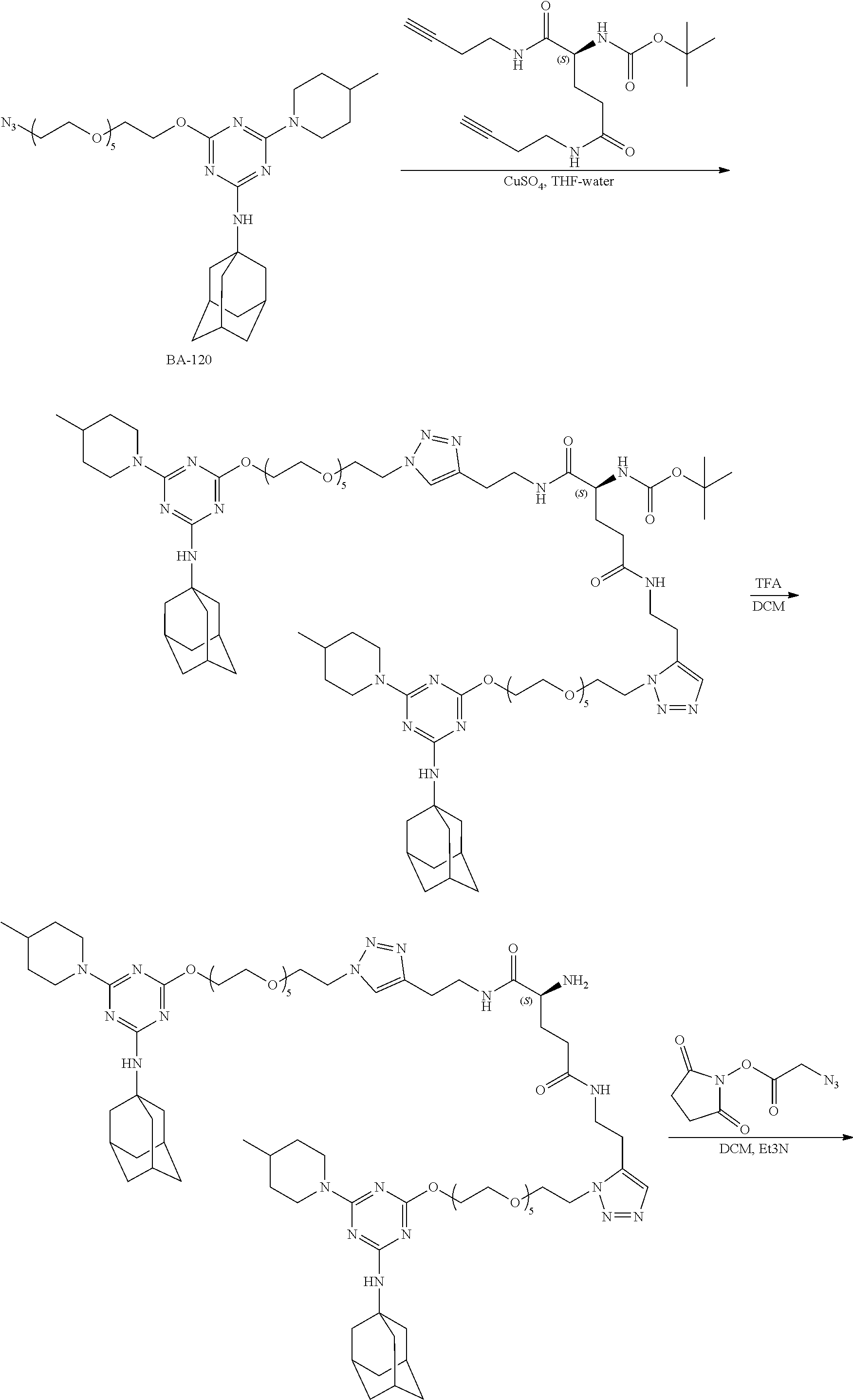

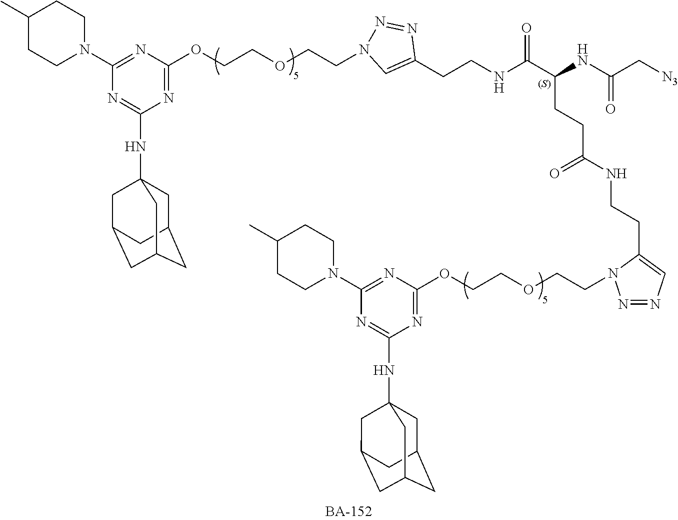

### Step 1: tert-butyl (S)-(1,5-bis((2-(1-(17-((4-(adamantan-1-ylamino)-6-(4-methylpiperidin-1-yl)-1,3,5-triazin-2-yl)oxy)-3,6,9,12,15-pentaoxaheptadecyl)-1H-1,2,3-triazol-4-yl)ethyl)amino)-1,5-dioxopentan-2-yl)carbamate

To a mixture of BA-120 N-(adamantan-1-yl)-4-((17-azido-3,6,9,12,15-pentaoxaheptadecyl)oxy)-6-(4-methylpiperidin-1-yl)-1,3,5-triazin-2-amine (0.19 g, 0.3 mmol) and bis-propargylamide (0.05 g, 0.143 mmol) in THF (0.79 ml) were successively added an aqueous solution of Cu₂SO₄ (0.003 g, 0.014 mmol,) in water (0.2 mL) dropwise, followed by an aqueous solution of sodium ascorbate (0.034 g, 0.172 mmol) in water (0.2 mL). The mixture was stirred for 14 hours at rt then concentrated. The residue was partitioned between DCM and water. The organic phase was separated, and the aqueous phase was extracted with DCM (3 times). The combined organic layers were washed with aq sat NaHCO₃, brine, dried, filtered, then concentrated. The residue was purified by silica-gel column chromatography using a gradient 0-20% methanol in ethyl acetate to afford the titled compound (121 mg, 52% yield). MS (ESI) m/z=1636.6 [M+Na]⁺.

### Step 2: (S)—N1,N5-bis(2-(1-(17-((4-(adamantan-1-ylamino)-6-(4-methylpiperidin-1-yl)-1,3,5-triazin-2-yl)oxy)-3,6,9,12,15-pentaoxaheptadecyl)-1H-1,2,3-triazol-4-yl)ethyl)-2-aminopentanediamide

To a solution of tert-butyl (S)-(1,5-bis((2-(1-(17-((4-(adamantan-1-ylamino)-6-(4-methylpiperidin-1-yl)-1,3,5-triazin-2-yl)oxy)-3,6,9,12,15-pentaoxaheptadecyl)-1H-1,2,3-triazol-4-yl)ethyl)amino)-1,5-dioxopentan-2-yl)carbamate (0.025 g, 0.015 mmol) in DCM (0.25 ml) at 0° C. was added TFA (0.125 mL) dropwise and the cooling bath was removed. The mixture was stirred for 5 hours at room temperature then concentrated under reduced pressure to afford the titled compound (26 mg, quant. yield). Reaction repeated identically on 100 mg scale and provided the titled compound (102 mg, quant. yield).

### Step 3: (S)—N1,N5-bis(2-(1-(17-((4-(adamantan-1-ylamino)-6-(4-methylpiperidin-1-yl)-1,3,5-triazin-2-yl)oxy)-3,6,9,12,15-pentaoxaheptadecyl)-1H-1,2,3-triazol-4-yl)ethyl)-2-(2-azidoacetamido)pentanediamide

NHS ester, 2,5-dioxopyrrolidin-1-yl 2-azidoacetate (19 mg, 97 μmol) was added in DCM (0.7 mL) at 0° C. to a solution of (S)—N1,N5-bis(2-(1-(17-((4-(adamantan-1-ylamino)-6-(4-methylpiperidin-1-yl)-1,3,5-triazin-2-yl)oxy)-3,6,9,12,15-pentaoxaheptadecyl)-1H-1,2,3-triazol-4-yl)ethyl)-2-aminopentanediamide (121 mg, 74 μmol) and DIEA (26 μL, 149 μmol) in DCM (0.7 mL). The mixture was stirred for 14 hours at room temperature, concentrated in vacuo and the residue was purified by silica-gel column using a gradient of 0-35% methanol in ethyl acetate to afford the titled compound (26 mg, 22% yield). MS (ESI): m/z=1619.7 [M+Na]⁺.

BA-152 was conjugated to an oligo sense strand according to general procedure type I. The product (MW: 8898.05 g/mol) was made with 90% purity and confirmed by HPLC and LCMS (m/z: 8896.61).

### Example 24: BA-157 Conjugate

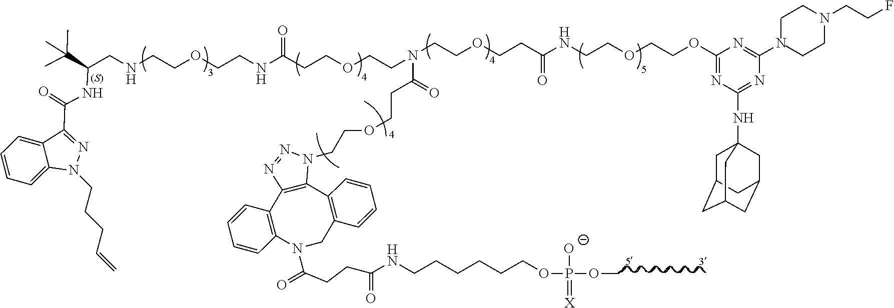

wherein X is O or S.

### BA-157: N—((S)-1-((4-(((3s,5s,7s)-adamantan-1-yl)amino)-6-(4-(2-fluoroethyl)piperazin-1-yl)-1,3,5-triazin-2-yl)oxy)-34-(1-azido-3,6,9,12-tetraoxapentadecan-15-oyl)-65,65-dimethyl-19,49,63-trioxo-3,6,9,12,15,22,25,28,31,37,40,43,46,53,56,59-hexadecaoxa-18,34,50,62-tetraazahexahexacontan-64-yl)-1-(pent-4-en-1-yl)-1H-indazole-3-carboxamide

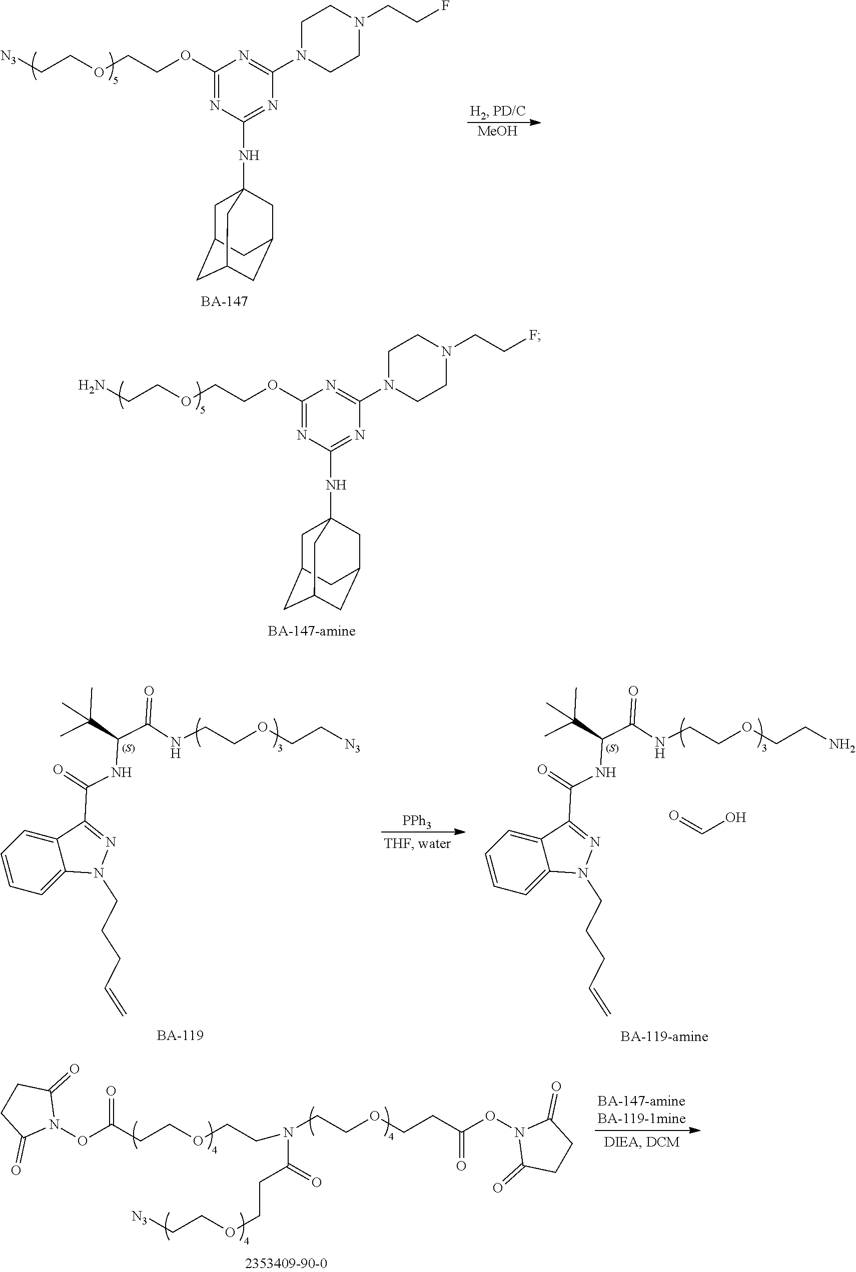

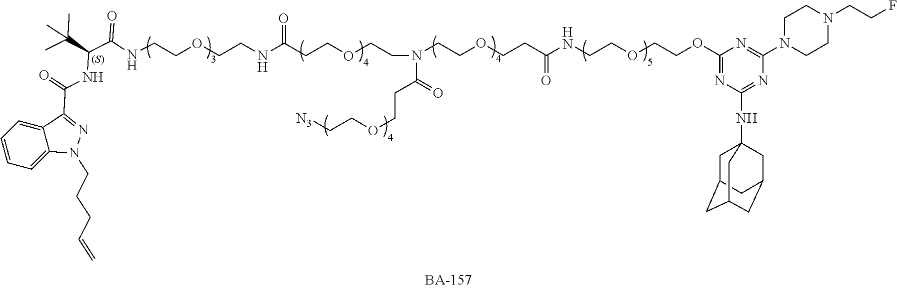

### Step 1: Reduction of BA-147; N-((3s,5s,7s)-adamantan-1-yl)-4-((17-amino-3,6,9,12,15-pentaoxaheptadecyl)oxy)-6-(4-(2-fluoroethyl)piperazin-1-yl)-1,3,5-triazin-2-amine

To a solution of N-((3s,5s,7s)-adamantan-1-yl)-4-((17-azido-3,6,9,12,15-pentaoxaheptadecyl)oxy)-6-(4-(2-fluoroethyl)piperazin-1-yl)-1,3,5-triazin-2-amine formate (0.13 g, 0.18 mmol) in methanol (2.6 mL) was added Pd/C (0.19 g, 0.18 mmol) under argon, and the suspension was stirred under 1 atm of H₂ (g) for 6 hours at room temperature. The catalyst was filtered off by celite and the filtrate was concentrated under reduced pressure. The residue was purified by reverse-phase using a gradient 0-30% acetonitrile in water (+0.1% formic acid) to afford the titled compound (51 mg, 38% yield). MS (ESI): m/z=640.7 [M+H]⁺.

### Step 2: Reduction of BA-119; (S)—N-(1-amino-15,15-dimethyl-13-oxo-3,6,9-trioxa-12-azahexadecan-14-yl)-1-(pent-4-en-1-yl)-1H-indazole-3-carboxamide

To a mixture of (S)—N-(1-azido-15,15-dimethyl-13-oxo-3,6,9-trioxa-12-azahexadecan-14-yl)-1-(pent-4-en-1-yl)-1H-indazole-3-carboxamide (0.28 g, 0.515 mmol, 1 eq) dissolved in THE (4.7 ml) was added PPh₃ (0.203 g, 0.772 mmol) followed by water (2.35 mL) dropwise at room temperature. The mixture was stirred overnight and concentrated under reduced pressure. The residue was purified by reverse-phase column chromatography (C18) using a gradient 0-35% ACN in water (+0.1% formic acid) to afford the titled compound as a formate salt (269 mg, 93% yield). MS (ESI): m/z=518.7 [M+H]⁺.

Step 3: Bis NHS ester bis(2,5-dioxopyrrolidin-1-yl) 16-(1-azido-3,6,9,12-tetraoxapentadecan-15-oyl)-4,7,10,13,19,22,25,28-octaoxa-16-azahentriacontanedioate (30 mg, 31 mol) was added at 0° C. to a solution of N-((3s,5s,7s)-adamantan-1-yl)-4-((17-amino-3,6,9,12,15-pentaoxaheptadecyl) oxy)-6-(4-(2-fluoroethyl)piperazin-1-yl)-1,3,5-triazin-2-amine diformate (50 mg, 68 μmol), (S)—N-(1-amino-15,15-dimethyl-13-oxo-3,6,9-trioxa-12-azahexadecan-14-yl)-1-(pent-4-en-1-yl)-1H-indazole-3-carboxamide (35 mg., 68 μmol), and DIEA (47 μL, 273 mol) in DCM (0.65 ml). The mixture was stirred for 14 hours at room temperature, concentrated, and purified by silica-gel column using a gradient 0-45% methanol in ethyl acetate to afford the titled compound in (17 mg, 13% yield). MS (ESI): m/z=1929.8 [M+Na]⁺.

BA-157 was conjugated to an oligo sense strand according to general procedure type I. The product (MW: 9208.40 g/mol) was made with 88% purity and confirmed by HPLC and LCMS (m/z: 9206.83).

### Example 25: Effect of RD2823 Targeting Rat CTNNB1 (Catenin Beta 1)

The compound RD2823 has the following compound attached to an siRNA targeting CTNNB1:

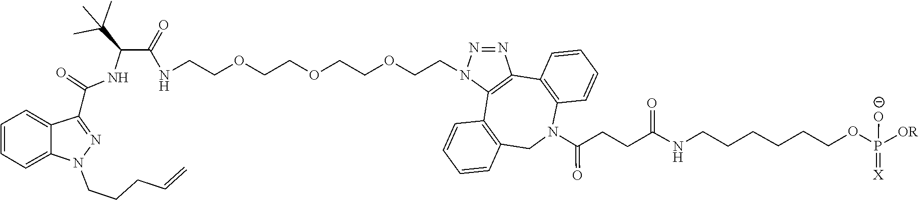

wherein X is S, and R is the siRNA targeting CTNNB1 (i.e., R is the covalently bound structure of compound RD2540 described below).

RD2823 was evaluated in an in vivo rat PD study. Six animals received a single 0.9 mg (3 mg/kg) dose via intrathecal injection on day 1. Animals were observed every day for behavioral changes. Frontal Cortex was collected from half the animals on day 15 and the other half at day 29. Tissue was immediately placed in homogenizing tube, snap frozen, and stored at −80° C. for gene expression analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl CTNNB1 TaqMan Gene Expression Assay (Thermo Fisher: Rn00584431_g1, FAM), 1 μl ACTB (VIC) TaqMan Gene Expression Assay (Thermo Fisher: Rn00667869_m1, VIC), and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the table below as percent inhibition of CTNNB1, relative to vehicle control.

TABLE 1
Average CTNNB1 Inhibition by Compound RD2823

| Compound | Day 15 | Day 29 |
|---|---|---|
| RD2823 | 69% | 56% |

### Example 26: Effect of RD2823 Targeting Rat CTNNB1 in Various Brain Regions

The compound RD2823 as described above was evaluated in an in vivo rat PD study. Ten animals received a single 0.9 mg (3 mg/kg) dose via intrathecal injection on day 1. Animals were observed every day for behavioral changes. Tissues were collected from half the animals on day 15 and the other half on day 29. Tissue was immediately placed in homogenizing tube, snap frozen, then kept at −80° C. for gene expression analysis.

RNA Isolation and qPCR was performed as described in Example 25 above. Results are presented in the table below as percent inhibition of CTNNB1, relative to vehicle control.

TABLE 2
Average CTNNB1 Inhibition by Compound RD2823

| Site | Day 15 | Day 29 |
|---|---|---|
| Striatum | 52% | 41% |
| Cerebellum | 65% | 59% |
| Brain Stem | 67% | 66% |
| Hippocampus | 74% | 66% |
| Frontal Cortex | 59% | 39% |
| Spinal Cord | 67% | 67% |

### Example 27: Effect of RD2540 Targeting Rat CTNNB1

The compound RD2540 has the structure described in the tables below. RD2540 is the siRNA used in Examples 25 and 26 above and Examples 28, 29, and 30 below and is not conjugated to a targeting ligand. The activity of RD2540 was tested as a comparison to the targeting ligand-conjugated compound tested above and below.

TABLE 3
Chemical Nomenclature

| Abbreviation | Structure |
|---|---|
| ‘v’ | 5′-vinylphosphonate modification |
| ‘m’ | 2′-O-methyl sugar modification (e.g., mA, mG, mC, mU) |
| ‘f’ | 2′-F sugar modification (e.g., fA, fG, fC, fU) |
| ‘*’ | Phosphorothioate internucleoside linkage |
| ‘dQ’ | Inverted abasic deoxyribose |

TABLE 4
Unconjugated Parent Compound

| | | Ref ID | SEQ ID |
|---|---|---|---|
| Compound | Modified Strands (5′-3′) | NO: | NO: |
| RD2540 | (vU)*(fU)*(mU)(mC)(mG)(fA)(mA)(fU)(fC)(mA) | IA0883 | 1 |
| | (mA)(mU)(mC)(fC)(mA)(fA)(mC)(mA)(mG)(mU) | | |
| | (mA)*(mG)*(mC) | | |
| | (mU)*(mA)*(mC)(mU)(mG)(mU)(fU)(mG)(fG)(fA) | IS1096 | 2 |
| | (fU)(mU)(mG)(mA)(mU)(mU)(mC)(mG)(mA) | | |
| | (mA)*(mA)*(dQ) | | |

RD2540 was evaluated in an in vivo rat PD study carried out as described in Example 26. Results are presented in the table below as percent inhibition of CTNNB1, relative to vehicle control.

TABLE 5
Average CTNNB1 Inhibition by Compound RD2540

| Compound | Day 15 | Day 29 |
|---|---|---|
| RD2540 | 31% | 13% |

### Example 28 (CTNNB1 Rat #001, BA-116): Effect of RD2545 Targeting Rat CTNNB1 (Catenin Beta 1)

The compound RD2545 has the following compound attached to an siRNA targeting CTNNB1:

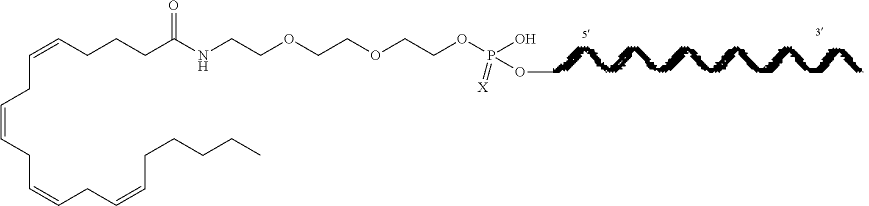

wherein X is S, and R is the siRNA targeting CTNNB1 (i.e., R is the covalently bound structure of compound RD2540 described above).

RD2545 was evaluated in an in vivo rat PD study. Six animals received a single 0.9 mg (3 mg/kg) dose via intrathecal injection on day 1. Animals were observed every day for behavioral changes. Frontal Cortex was collected from half the animals on day 15 and the other half at day 29. Tissues was immediately placed in homogenizing tube, snap frozen, and stored at −80° C. for gene expression analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl CTNNB1 TaqMan Gene Expression Assay (Thermo Fisher: Rn00584431_g1, FAM), 1 μl ACTB (VIC) TaqMan Gene Expression Assay (Thermo Fisher: Rn00667869_m1, VIC), and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the table below as percent inhibition of CTNNB1, relative to vehicle control.

TABLE 6
Average CTNNB1 Inhibition by Compound RD2545

| Compound | Day 15 | Day 29 |
|---|---|---|
| RD2545 | 28% | 22% |

### Example 29 (CTNNB1 Rat #004, BA-120): Effect of RD2824 Targeting Rat CTNNB1 (Catenin Beta 1)

The compound RD2824 has the following compound attached to an siRNA targeting CTNNB1:

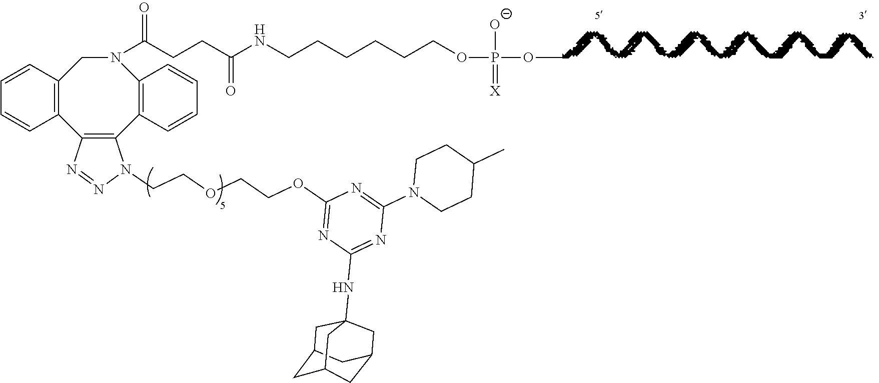

wherein X is S, and R is the siRNA targeting CTNNB1 (i.e., R is the covalently bound structure of compound RD2540 described below).

RD2824 was evaluated in an in vivo rat PD study. Six animals received a single 0.9 mg (3 mg/kg) dose via intrathecal injection on day 1. Animals were observed every day for behavioral changes. Frontal Cortex was collected from half the animals on day 15 and the other half at day 29. Tissue was immediately placed in homogenizing tube, snap frozen, and stored at −80° C. for gene expression analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl CTNNB1 TaqMan Gene Expression Assay (Thermo Fisher: Rn00584431_g1, FAM), 1 μl ACTB (VIC) TaqMan Gene Expression Assay (Thermo Fisher: Rn00667869_m1, VIC), and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the table below as percent inhibition of CTNNB1, relative to vehicle control.

TABLE 7
Average CTNNB1 Inhibition by Compound RD2824

| Compound | Day 15 | Day 29 |
|---|---|---|
| RD2824 | 46% | 61% |

### Example 30 (CTNNB1 Rat #005, BA-130): Effect of RD2953 Targeting Rat CTNNB1 (Catenin Beta 1)

The compound RD2953 has the following compound attached to an siRNA targeting CTNNB1:

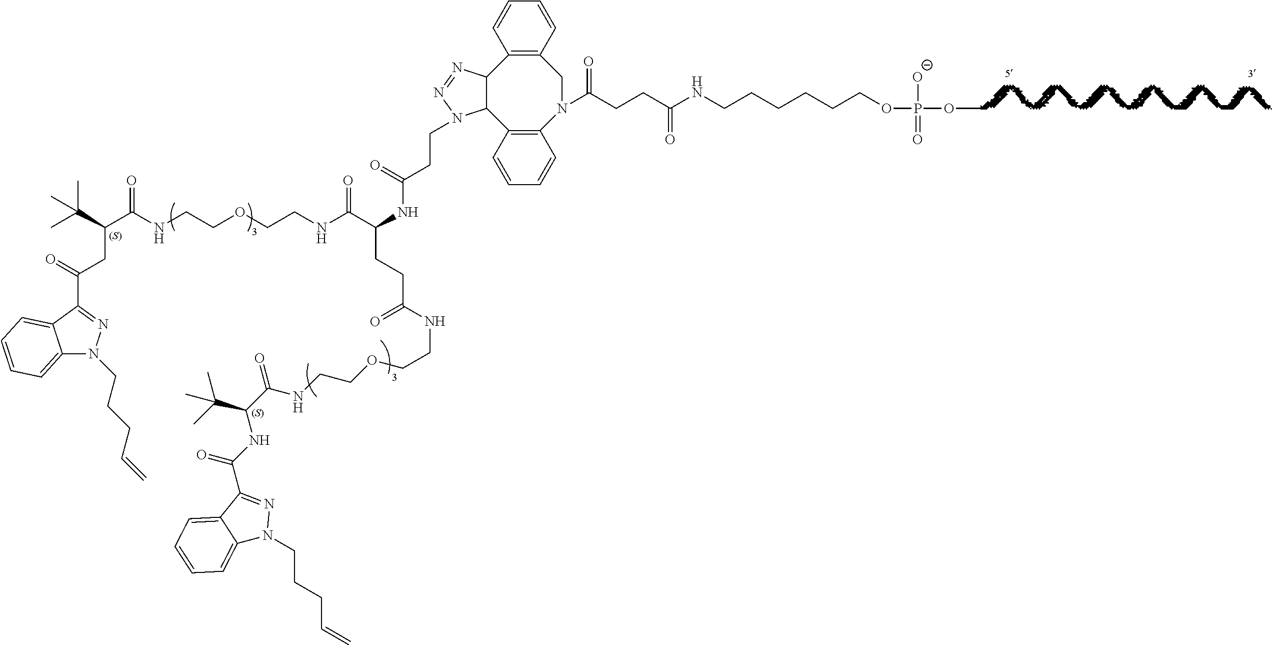

wherein X is S, and R is the siRNA targeting CTNNB1 (i.e., R is the covalently bound structure of compound RD2540 described below).

RD2953 was evaluated in an in vivo rat PD study. Six animals received a single 0.9 mg (3 mg/kg) dose via intrathecal injection on day 1. Animals were observed every day for behavioral changes. Frontal Cortex was collected from half the animals on day 15 and the other half at day 29. Tissue was immediately placed in homogenizing tube, snap frozen, and stored at −80° C. for gene expression analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl CTNNB1 TaqMan Gene Expression Assay (Thermo Fisher: Rn00584431_g1, FAM), 1 μl ACTB (VIC) TaqMan Gene Expression Assay (Thermo Fisher: Rn00667869_m1, VIC), and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the table below as percent inhibition of CTNNB1, relative to vehicle control.

TABLE 8
Average CTNNB1 Inhibition by Compound RD2953

| Compound | Day 15 | Day 29 |
|---|---|---|
| RD2953 | 64% | 64% |

### Example 31 (Target A Rat #3, BA-119): Effect of Compound 1 Targeting Rat Target A in Various Brain Regions

The Compound 1 has an siRNA targeting Target A attached to the following CB1 ligand:

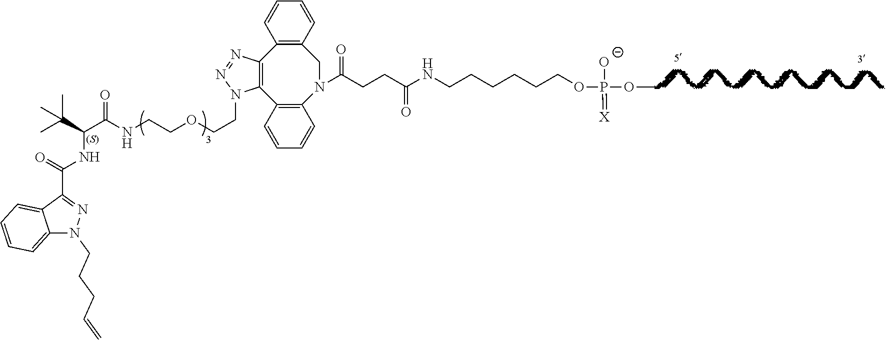

wherein X is S, and R is the siRNA targeting Target A (i.e., R is the covalently bound structure of the siRNA).

Compound 1 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, day 29, or day 43, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table 9 below as percent inhibition of Target A, relative to vehicle control.

TABLE 9
Average Target A Inhibition by Compound 1

| | Day | Day | Day |
|---|---|---|---|
| Brain Region | 15 | 29 | 43 |
| Striatum | 72% | 56% | 59% |
| Hippocampus | 76% | 84% | 76% |
| Frontal Cortex | 83% | 93% | 78% |
| Temporal Cortex | 77% | 92% | 71% |
| Cerebellum | 79% | 84% | 72% |
| Brainstem | 84% | 88% | 80% |
| Cervical Spinal Cord | 91% | 86% | 85% |
| Thoracic Spinal Cord | 89% | 87% | 82% |
| Lumbar Spinal Cord | 83% | 88% | 90% |

### Example 32 (Target A Rat #4, BA-130): Effect of Compound 2 Targeting Rat Target A in Various Brain Regions

The Compound 2 has an siRNA targeting Target A attached to the following CB1 ligand:

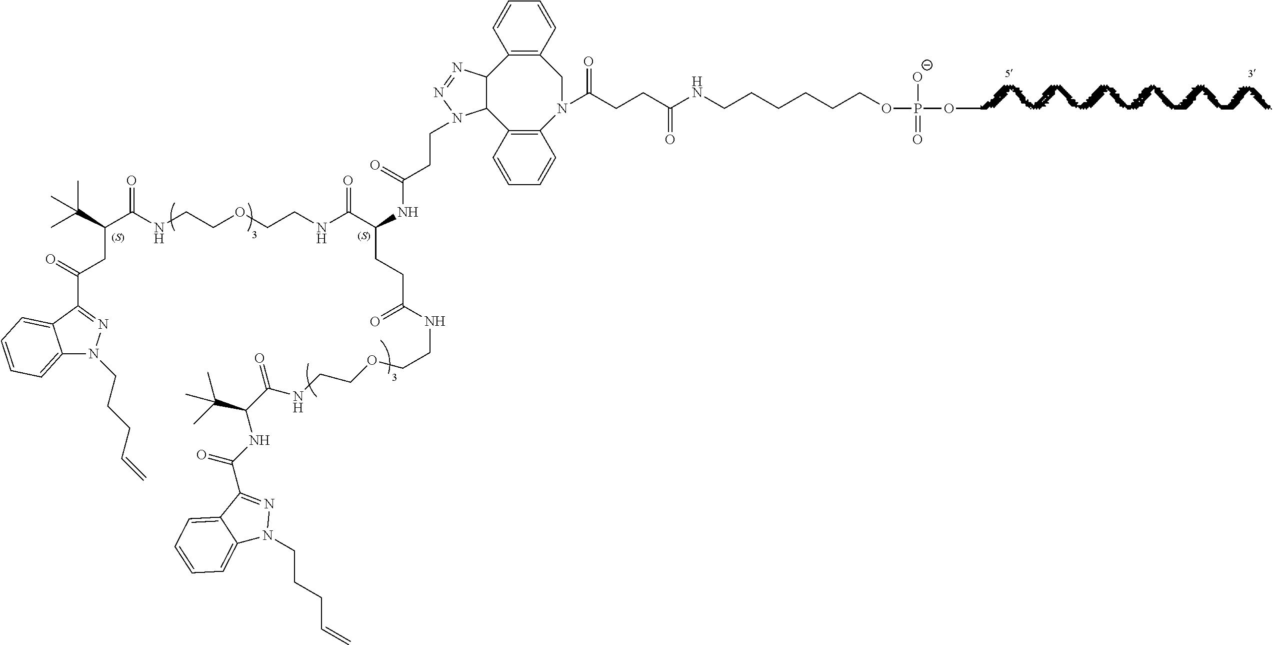

wherein X is S, and R is the siRNA targeting Target A (i.e., R is the covalently bound structure of the siRNA).

Compound 2 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15 or day 29, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 10
Average Target A Inhibition by Compound 2

| | Day | Day |
|---|---|---|
| Brain Region | 15 | 29 |
| Striatum | 56% | 60% |
| Hippocampus | 80% | 71% |
| Frontal Cortex | 84% | 92% |
| Temporal Cortex | 82% | 84% |
| Cerebellum | 74% | 82% |
| Brainstem | 77% | 84% |
| Cervical Spinal Cord | 76% | 90% |
| Thoracic Spinal Co | 74% | 91% |
| Lumbar Spinal Cord | 81% | 95% |

### Example 33 (Target A Rat #4, BA-138): Effect of Compound 3 Targeting Rat Target A in Various Brain Regions

The Compound 3 has an siRNA targeting Target A attached to the following CB₁ ligand:

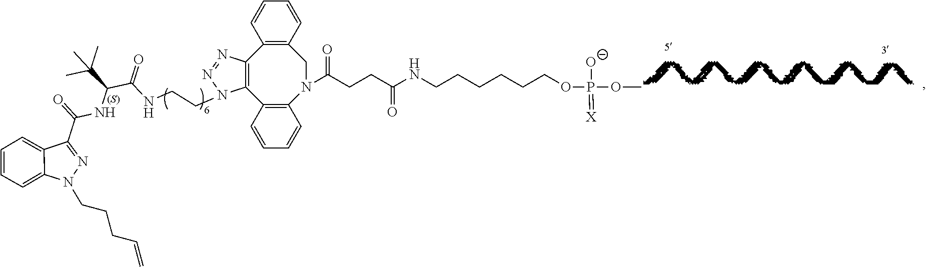

wherein X is S, and R is the siRNA targeting Target A (i.e., R is the covalently bound structure of the siRNA).

Compound 3 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15 or day 29, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 11
Average Target A Inhibition by Compound 3

| | Day | Day |
|---|---|---|
| Brain Region | 15 | 29 |
| Striatum | 41% | 21% |
| Hippocampus | 68% | 36% |
| Frontal Cortex | 67% | 46% |
| Temporal Cortex | 65% | 43% |
| Cerebellum | 72% | 75% |
| Brainstem | 85% | 86% |
| Cervical Spinal Cord | 79% | 87% |
| Thoracic Spinal Co | 70% | 86% |
| Lumbar Spinal Cord | 77% | 92% |

### Example 34 (Target A Rat #7, BA-120): Effect of Compound 4 Targeting Rat Target A in Various Brain Regions

The Compound 4 has an siRNA targeting Target A attached to the following CB1 ligand:

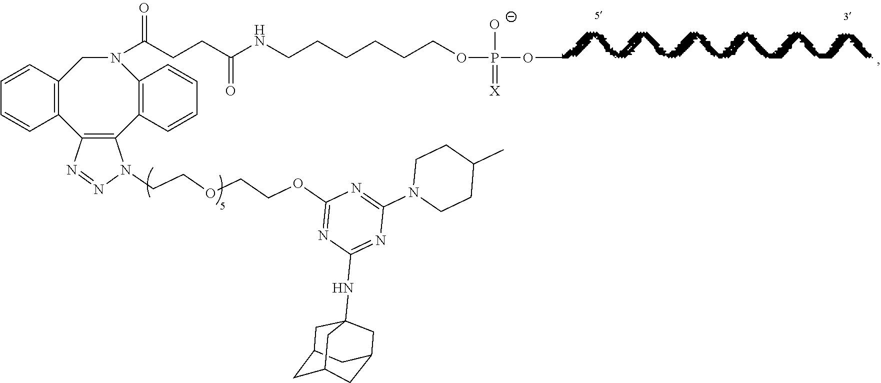

wherein X is S, and R is the siRNA targeting Target A (i.e., R is the covalently bound structure of the siRNA).

Compound 4 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 12
Average Target A Inhibition by Compound 4

| | Day |
|---|---|
| Brain Region | 15 |
| Striatum | 50% |
| Hippocampus | 43% |
| Frontal Cortex | 81% |

### Example 35 (Target a Rat #7, BA-152): Effect of Compound 5 Targeting Rat Target a in Various Brain Regions

The Compound 5 has an siRNA targeting Target A attached to the following CB1 ligand:

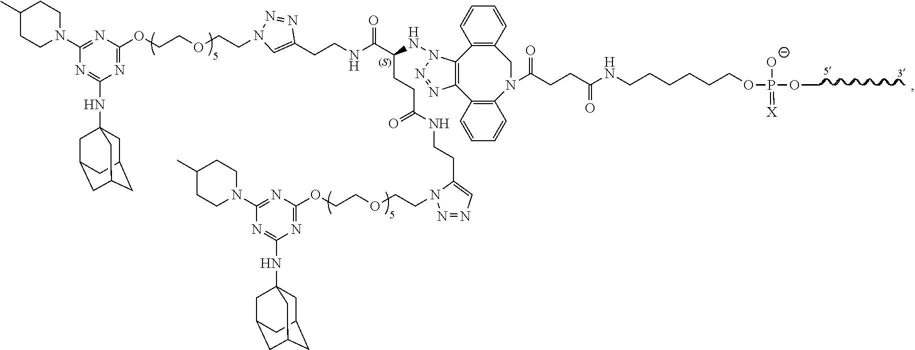

wherein X is S, and R is the siRNA targeting Target A (i.e., R is the covalently bound structure of the siRNA).

Compound 5 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 13
Average Target A Inhibition by Compound 5

| | Day |
|---|---|
| Brain Region | 15 |
| Striatum | 52% |
| Hippocampus | 53% |
| Frontal Cortex | 71% |

### Example 36 (Target A Rat #7, BA-157): Effect of Compound 6 Targeting Rat Target A in Various Brain Regions

The Compound 6 has an siRNA targeting Target A attached to the following CB1 ligand:

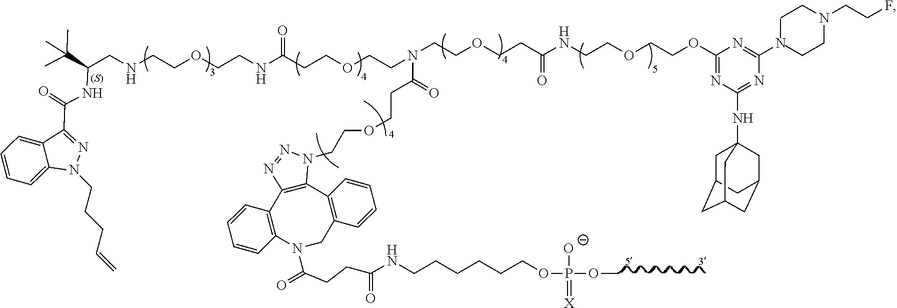

wherein X is S, and R is the siRNA targeting Target A (i.e., R is the covalently bound structure of the siRNA).

Compound 6 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 14
Average Target A Inhibition by Compound Compound 6

| | Day |
|---|---|
| Brain Region | 15 |
| Striatum | 55% |
| Hippocampus | 54% |
| Frontal Cortex | 62% |

### Example 37 (Target A Rat #7, BA-153): Effect of Compound 7 Targeting Rat Target A in Various Brain Regions

The Compound 7 has an siRNA targeting Target A attached to the following CB1 ligand:

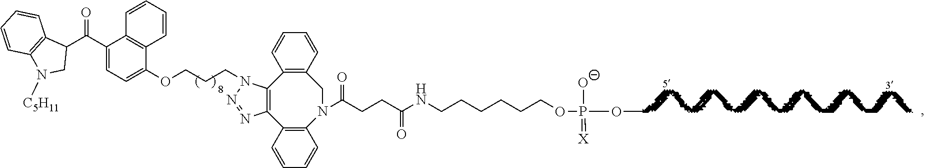

wherein X is S, and R is the siRNA targeting Target A (i.e., R is the covalently bound structure of the siRNA).

Compound 7 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 15
Average Target A Inhibition by Compound Compound 7

| Brain Region | Day 15 |
|---|---|
| Striatum | 35% |
| Hippocampus | 81% |
| Frontal Cortex | 41% |

### Example 38 (Target A Rat #7, BA-156): Effect of Compound 8 Targeting Rat Target A in Various Brain Regions

The Compound 8 has an siRNA targeting Target A attached to the following CB1 ligand:

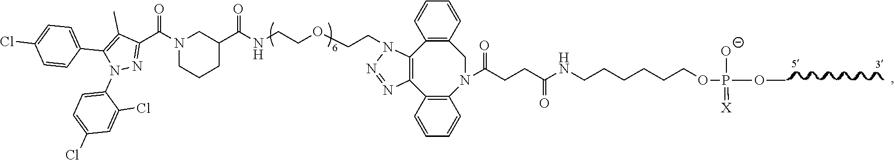

wherein X is S, and R is the siRNA targeting Target A (i.e., R is the covalently bound structure of the siRNA).

Compound 8 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 16
Average Target A Inhibition by Compound 8

| Brain Region | Day 15 |
|---|---|
| Striatum | 57% |
| Hippocampus | 89% |
| Frontal Cortex | 63% |

### Example 39 (Target A Rat #7, BA-146): Effect of Compound 9 Targeting Rat Target A in Various Brain Regions

The Compound 9 has an siRNA targeting Target A attached to the following CB1 ligand:

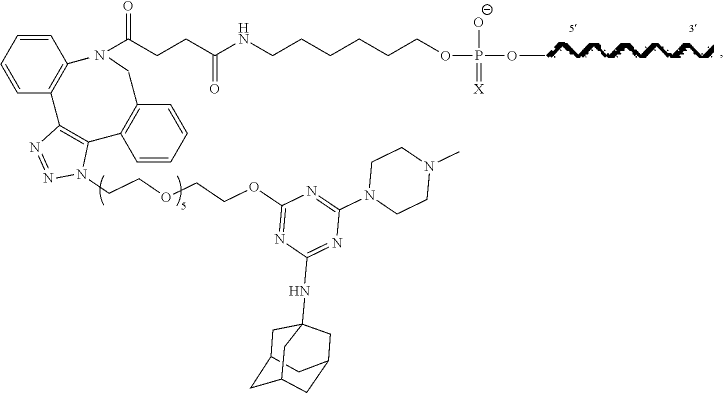

wherein X is S, and R is the siRNA targeting Target A (i.e., R is the covalently bound structure of the siRNA).

Compound 9 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 17
Average Target A Inhibition by Compound 9

| Brain Region | Day 15 |
|---|---|
| Striatum | 66% |
| Hippocampus | 90% |
| Frontal Cortex | 80% |

### Example 40 (Target A Rat #7, BA-147): Effect of Compound 10 Targeting Rat Target A in Various Brain Regions

The Compound 10 has an siRNA targeting Target A attached to the following CB1 ligand:

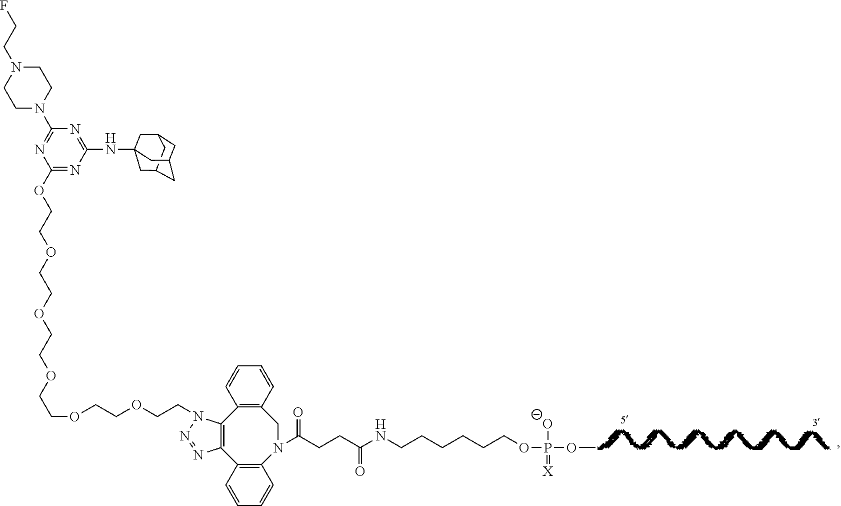

wherein X is S, and R is the siRNA targeting Target A (i.e., R is the covalently bound structure of the siRNA).

Compound 10 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 18
Average Target A Inhibition by Compound 10

| Brain Region | Day 15 |
|---|---|
| Striatum | 51% |
| Hippocampus | 89% |
| Frontal Cortex | 58% |

### Example 41 (Target A Rat #7, BA-155): Effect of Compound 11 Targeting Rat Target A in Various Brain Regions

The Compound 11 has an siRNA targeting Target A attached to the following CB1 ligand:

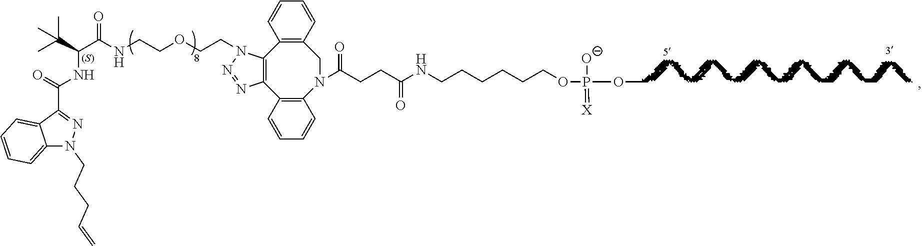

wherein X is S, and R is the siRNA targeting Target A (i.e., R is the covalently bound structure of the siRNA provided in Table 9 above).

Compound 11 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exception that instead of rat CTNNB1 TaqMan Gene Expression Assay, the rat Target A TaqMan Gene Expression Assay (Thermo Fisher) was used. Results are presented in Table below as percent inhibition of Target A, relative to vehicle control.

TABLE 19
Average Target A Inhibition by Compound 11

| Brain Region | Day 15 |
|---|---|
| Striatum | 51% |
| Hippocampus | 91% |
| Frontal Cortex | 61% |

### Example 42 (Target B Mouse #27, BA-120/BA-120): Effect of Compound 12 Targeting Human Target B in Various Brain Regions

The Compound 12 has an siRNA targeting human Target B attached to the following CB1 ligand:

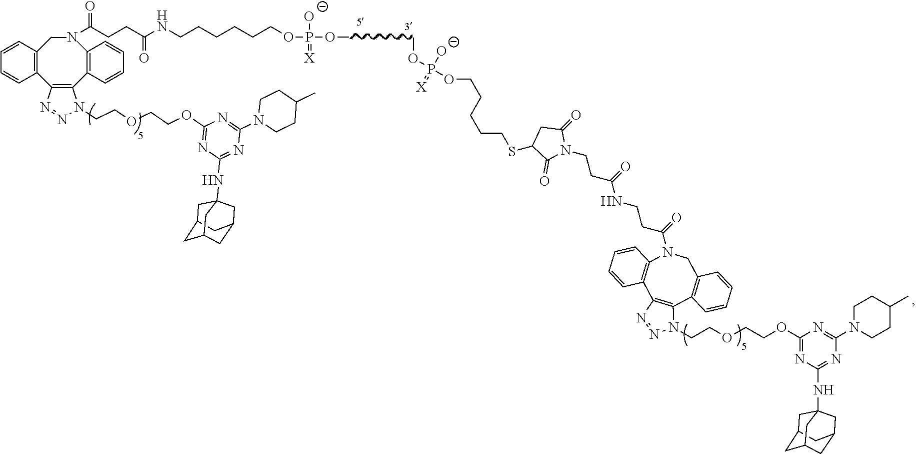

wherein X is S, and R is the siRNA targeting human Target B (i.e., R is the covalently bound structure of the siRNA).

Compound 12 was evaluated in an in vivo human Target B transgenic mice PD study. The animals received a single vehicle or 0.2 mg (10 mg/kg) dose via intracerebroventricular injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 25 above, with the exceptions that the instead of rat CTNNB1 TaqMan Gene Expression Assay, the human Target B TaqMan Gene Expression Assay (Thermo Fisher) was used; instead of rat ACTB (VIC) TaqMan Gene Expression Assay, the mouse GAPDH TaqMan Gene Expression Assay (Thermo Fisher: Mm99999915_g1, VIC) was used. Results are presented in table below as percent inhibition of human Target B, relative to vehicle control.

TABLE 20
Average Target B Inhibition by Compound 12

| Brain Region | Day 15 |
|---|---|
| Cortex | 41% |
| Hippocampus | 68% |
| Cerebellum | 58% |
| Striatum | 60% |
| Cervical Spinal Cord | 76% |

### Example 43 (Human Target B Mouse #33, BA-168): Effect of Compound 13 Targeting Human Target B in Various Brain Regions

The Compound 13 has an siRNA targeting human Target B attached to the following CB1 ligand:

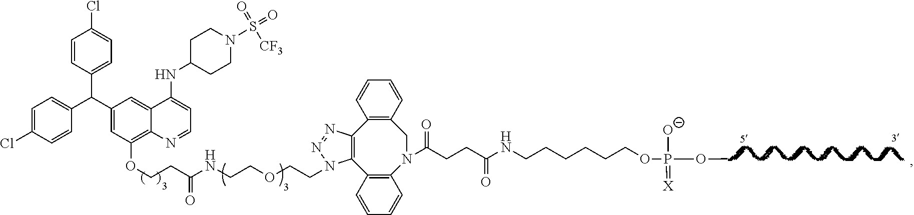

wherein X is S, and R is the siRNA targeting Target B (i.e., R is the covalently bound structure of the siRNA).

Compound 13 was evaluated in an in vivo human Target B transgenic mice PD study carried out as described in Example 42. Results are presented in table below as percent inhibition of Target B, relative to vehicle control.

TABLE 21
Average Target B Inhibition by Compound 13

| Brain Region | Day 15 |
|---|---|
| Cortex | 52% |
| Hippocampus | 71% |
| Cerebellum | 47% |
| Striatum | 66% |
| Cervical Spinal Cord | 72% |

### Example 44 (Human Target B Mouse #36, BA-176): Effect of Compound 14 Targeting Human Target B in Various Brain Regions

The Compound 14 has an siRNA targeting human Target B attached to the following CB1 ligand:

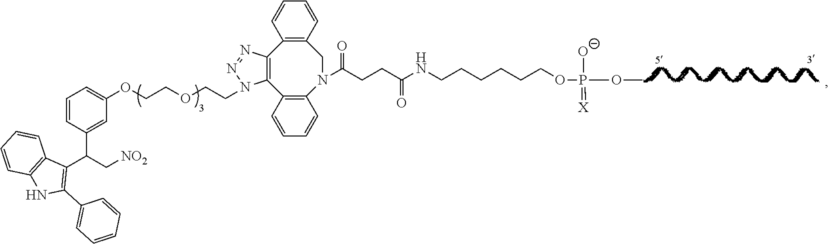

wherein X is S, and R is the siRNA targeting Target B (i.e., R is the covalently bound structure of the siRNA).

Compound 14 was evaluated in an in vivo human Target B transgenic mice PD study carried out as described in Example 42. Results are presented in table below as percent inhibition of Target B, relative to vehicle control.

TABLE 22
Average Target B Inhibition by Compound 14

| Brain Region | Day 15 |
|---|---|
| Cortex | 56% |
| Hippocampus | 71% |
| Cerebellum | 58% |
| Striatum | 73% |
| Cervical Spinal Cord | 50% |

### Example 45 (Human Target B Mouse #36, BA-177): Effect of Compound 15 Targeting Human Target B in Various Brain Regions

The Compound 15 has an siRNA targeting human Target B attached to the following CB1 ligand:

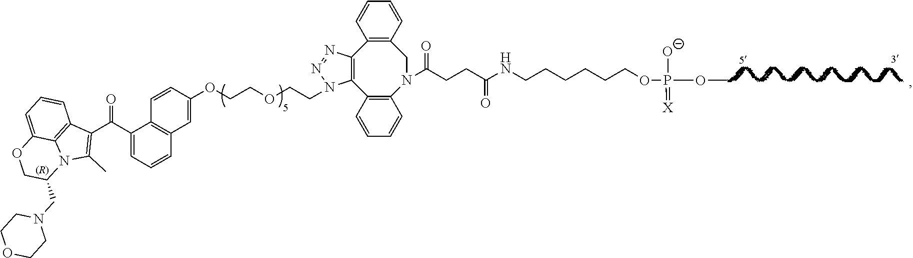

wherein X is S, and R is the siRNA targeting Target B (i.e., R is the covalently bound structure of the siRNA).

Compound 15 was evaluated in an in vivo human Target B transgenic mice PD study carried out as described in Example 42. Results are presented in table below as percent inhibition of Target B, relative to vehicle control.

TABLE 23
Average Target B Inhibition by Compound 15

| Brain Region | Day 15 |
|---|---|
| Cortex | 63% |
| Hippocampus | 76% |
| Cerebellum | 54% |
| Striatum | 71% |
| Cervical Spinal Cord | 15% |

### Example 46 (Human Target B Mouse #39, BA-120): Effect of Compound 16 Targeting Human Target B in Various Brain Regions

The Compound Target B has an siRNA targeting human Target B attached to the following CB1 ligand:

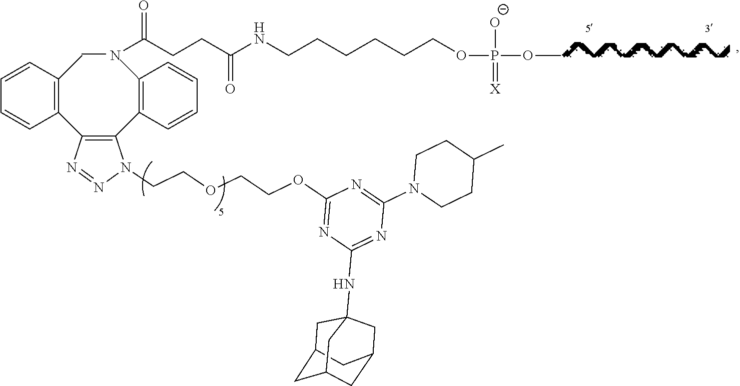

wherein X is S, and R is the siRNA targeting Target i.e., is the covalently bound structure of the siRNA).

Compound 16 was evaluated in an in vivo human Target B transgenic mice PD study carried out as described in Example 42. Results are presented in table below as percent inhibition of Target B, relative to vehicle control.

TABLE 24
Average Target B Inhibition by Compound 16

| Brain Region | Day 15 |
|---|---|
| Cortex | 29% |
| Hippocampus | 18% |
| Cerebellum | 42% |
| Striatum | 35% |
| Cervical Spinal Cord | 41% |

### Example 47 (Human Target B Mouse #23, BA-163): Effect of Compound 17 Targeting Human Target B in Various Brain Regions

The Compound 17 has an siRNA targeting human Target B attached to the following CB1 ligand:

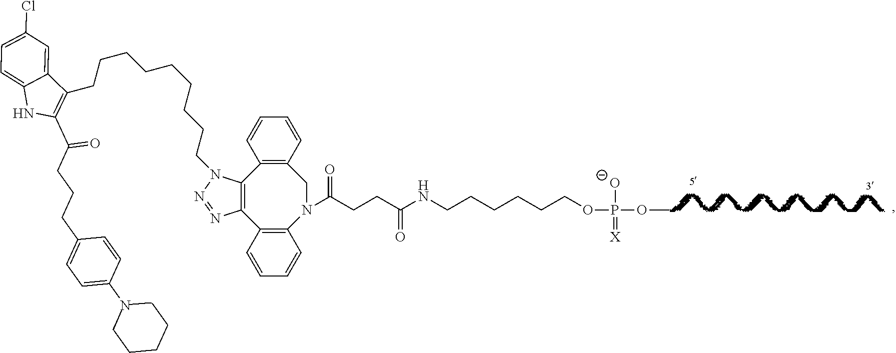

wherein X is S, and R is the siRNA targeting Target B (i.e., R is the covalently bound structure of the siRNA).

Compound 17 was evaluated in an in vivo human Target B transgenic mice PD study carried out as described in Example 42, except that instead of intracerebroventricular injection, intracisternal magna injection was performed. Results are presented in table below as percent inhibition of Target B, relative to vehicle control.

TABLE 25
Average Target B Inhibition by Compound 17

| Brain Region | Day 15 |
|---|---|
| Cortex | 43% |
| Hippocampus | 45% |
| Cerebellum | 35% |
| Striatum | 24% |
| Cervical Spinal Cord | 61% |

### Example 48 (Human Target B Mouse #10, BA-119): Effect of Compound 18 Targeting Human Target B in Various Brain Regions

The Compound 18 has an siRNA targeting human Target B attached to the following CB1 ligand:

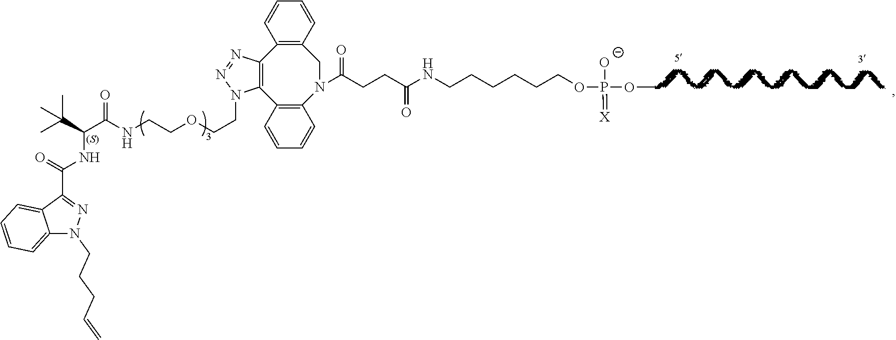

wherein X is S, and R is the siRNA targeting Target B (i.e., R is the covalently bound structure of the siRNA).

Compound 18 was evaluated in an in vivo human Target B transgenic mice PD study carried out as described in Example 42, except that instead of intracerebroventricular injection, intracisternal magna injection was performed. Results are presented in table below as percent inhibition of Target B, relative to vehicle control.

TABLE 26
Average Target B Inhibition by Compound 18

| Brain Region | Day 15 |
|---|---|
| Cortex | 36% |
| Hippocampus | 25% |
| Cerebellum | 0% |
| Striatum | 20% |
| Brainstem | 52% |
| Cervical Spinal Cord | 31% |
| Thoracic Spinal Cord | 33% |
| Lumbar Spinal Cord | 40% |

### Example 49 (Human Target B Mouse #15, BA-146): Effect of Compound 19 Targeting Human Target B in Various Brain Regions

The Compound 19 has an siRNA targeting human Target B attached to the following CB1 ligand:

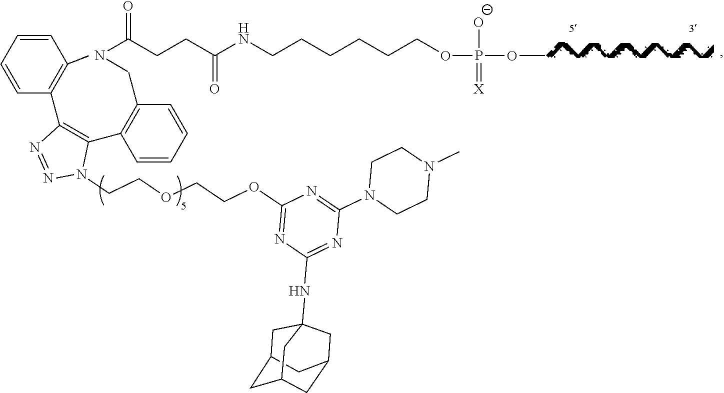

wherein X is S, and R is the siRNA targeting Target B (i.e., R is the covalently bound structure of the siRNA).

Compound 19 was evaluated in an in vivo human Target B transgenic mice PD study carried out as described in Example 42, except that instead of intracerebroventricular injection, intracisternal magna injection was performed. Results are presented in table below as percent inhibition of Target B, relative to vehicle control.

TABLE 27
Average Target B Inhibition by Compound 19

| Brain Region | Day 15 |
|---|---|
| Cortex | 29% |
| Hippocampus | 31% |
| Cerebellum | 18% |
| Striatum | 17% |
| Brainstem | 55% |
| Cervical Spinal Cord | 40% |
| Thoracic Spinal Cord | 24% |
| Lumbar Spinal Cord | 10% |

### Example 50 (Human Target B Mouse #23, BA-164): Effect of Compound 20 Targeting Human Target B in Various Brain Regions

The Compound 20 has an siRNA targeting human Target B attached to the following CB1 ligand:

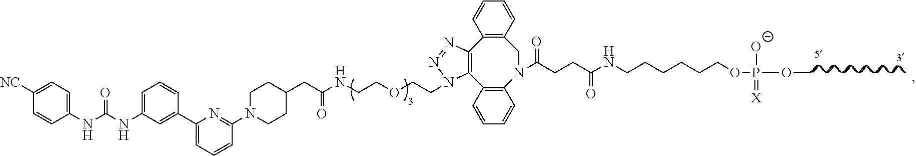

wherein X is S, and R is the siRNA targeting Target B (i.e., R is the covalently bound structure of the siRNA).

Compound 20 was evaluated in an in vivo human Target B transgenic mice PD study carried out as described in Example 42, except that instead of intracerebroventricular injection, intracisternal magna injection was performed. Results are presented in table below as percent inhibition of Target B, relative to vehicle control.

TABLE 28
Average Target B Inhibition by Compound 20

| Brain Region | Day 15 |
|---|---|
| Cortex | 36% |
| Hippocampus | 32% |
| Cerebellum | 39% |
| Striatum | 28% |
| Cervical Spinal Cord | 58% |

### Example 51 (Human Target B Mouse #23, BA-165): Effect of Compound 21 Targeting Human Target B in Various Brain Regions

The Compound 21 has an siRNA targeting human Target B attached to the following CB1 ligand:

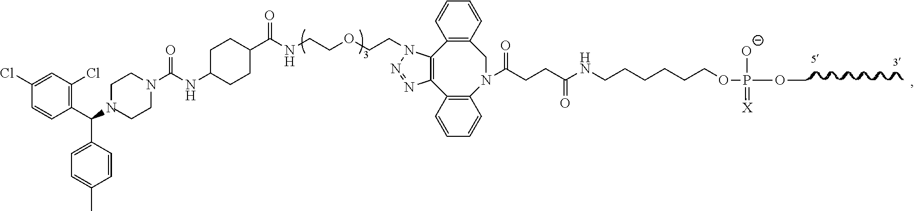

wherein X is S, and R is the siRNA targeting Target B (i.e., R is the covalently bound structure of the siRNA provided in Table 21).

Compound 21 was evaluated in an in vivo human Target B transgenic mice PD study carried out as described in Example 42, except that instead of intracerebroventricular injection, intracisternal magna injection was performed. Results are presented in table below as percent inhibition of Target B, relative to vehicle control.

TABLE 29
Average Target B Inhibition by Compound 21

| Brain Region | Day 15 |
|---|---|
| Cortex | 48% |
| Hippocampus | 32% |
| Cerebellum | 51% |
| Striatum | 32% |
| Cervical Spinal Cord | 64% |

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize or be able to ascertain using no more than routine experimentation, many equivalents of the specific embodiments described herein. Such equivalents are intended to be encompassed by the following claims.

What is claimed is:

1. A compound, or a stereoisomer, tautomer, prodrug, or salt thereof, comprising the structure of Formula (I′): 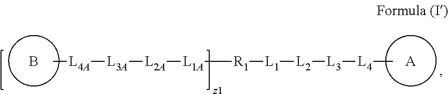
wherein:
each of 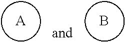
is independently a cannabinoid receptor type 1 (CB₁) ligand;
each of L₁, L₂, L, L₄, L_{1A}, L_{2A}, L_{3A}, and L_{4A} is independently a linker, a bond, or absent;
R¹ comprises one or more oligonucleotides, protecting groups, small molecules, proteins, antibodies, and/or peptides; and
z1 is 0 or 1.

2. The compound, or a stereoisomer, tautomer, prodrug, or salt thereof, of claim 1, wherein the compound comprises the structure of Formula (I″): 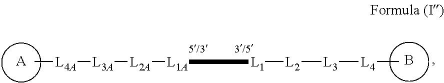
wherein: [symbol]
is an oligonucleotide.

3. The compound, or a stereoisomer, tautomer, prodrug, or salt thereof, of claim 2, wherein the compound comprises the structure of Formula (I″-a): 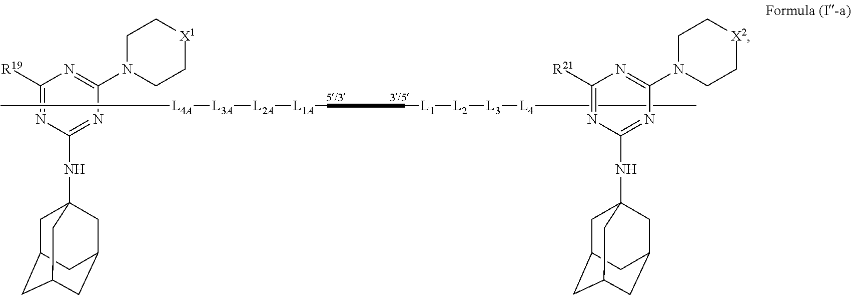
wherein:
X¹ is NR¹⁰ or CR¹¹R¹²;
X² is NR¹³ or CR¹⁴R¹⁵;
R¹⁰, R¹¹, R¹², R¹³, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D};
—NR^{19B}C(O)OR^{19D}, NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, —SOₙ₂₁R^{21A}, —SOᵥ₂₁NR^{21B}R^{21C}, —NHNR^{21B}R^{21C}, —ONR^{21B}R^{21C}, —NHC(O)NHNR^{21B}R^{21C}, —NHC(O)NR^{21B}R^{21C}, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D}, R^{21A}, R^{21B}, R^{21C} and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C}; and R^{21B} and R^{21C} substituents bonded to the same nitrogen atom may optionally be joined to form a substituted or unsubstituted heterocycloalkyl or substituted or unsubstituted heteroaryl;
n19 and n21 are each independently 0, 1, 2, 3, or 4; and
v19 and v21 are each independently 1 or 2.

4. The compound, or a stereoisomer, tautomer, prodrug, or salt thereof, of claim 3, wherein the compound comprises the structure of Formula (I″-a-1):

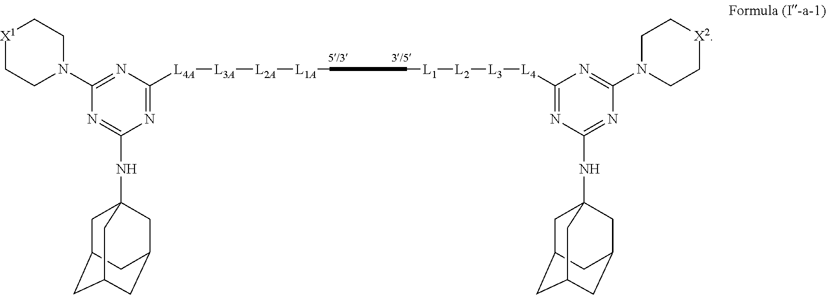

5. The compound, or a stereoisomer, tautomer, prodrug, or salt thereof, of claim 3, wherein the compound comprises the structure of Formula (I″-a-2):

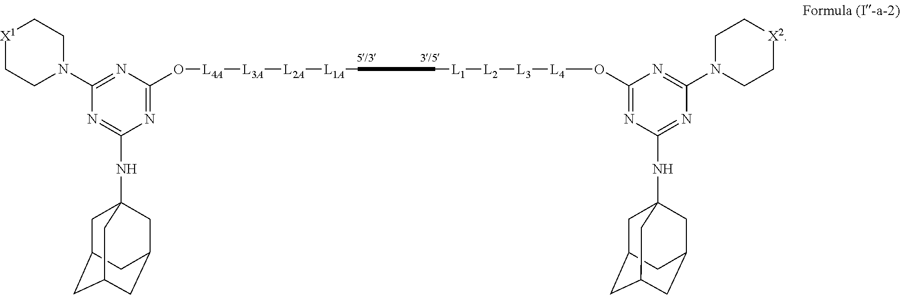

6. The compound, or a stereoisomer, tautomer, prodrug, or salt thereof, of claim 2, wherein the compound comprises the structure of Formula (I″-b):

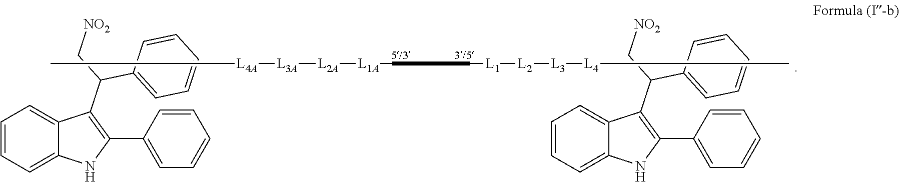

7. The compound, or a stereoisomer, tautomer, prodrug, or salt thereof, of claim 6, wherein the compound comprises the structure of Formula (I″-b-1):

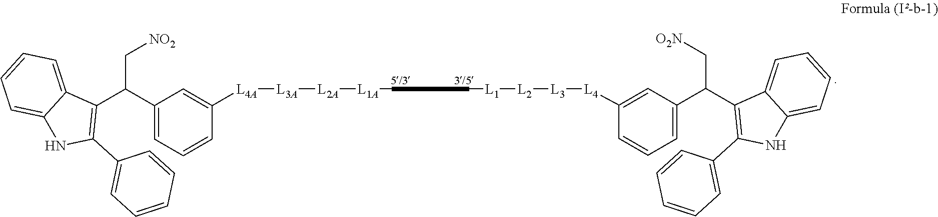

8. The compound, or a stereoisomer, tautomer, prodrug, or salt thereof, of claim 6, wherein the compound comprises the structure of Formula (I″-b-2):

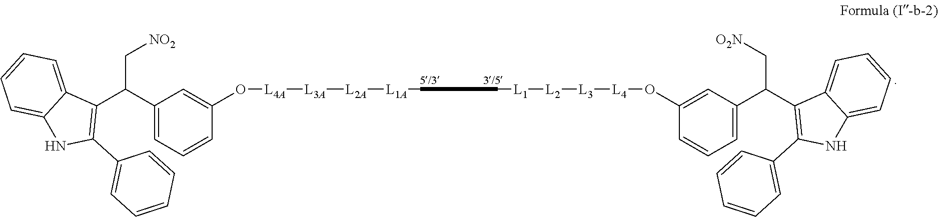

9. The compound, or a stereoisomer, tautomer, prodrug, or salt thereof, of claim 2, wherein the compound comprises the structure of Formula (I″-c):

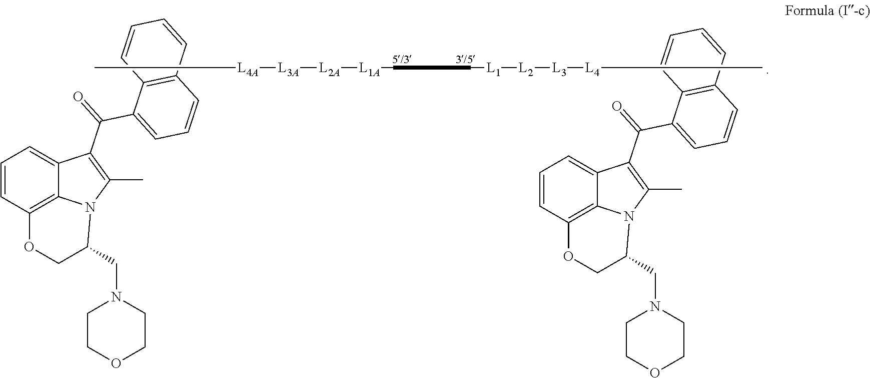

10. The compound, or a stereoisomer, tautomer, prodrug, or salt thereof, of claim 9, wherein the compound comprises the structure of Formula (I″-c-1):

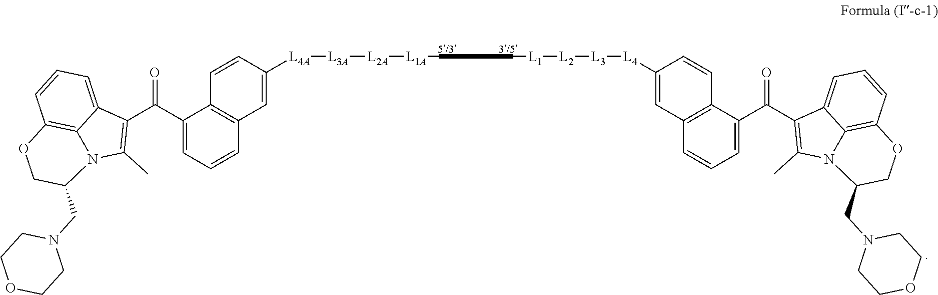

11. The compound, or a stereoisomer, tautomer, prodrug, or salt thereof, of claim 9, wherein the compound comprises the structure of Formula (I″-c-2):

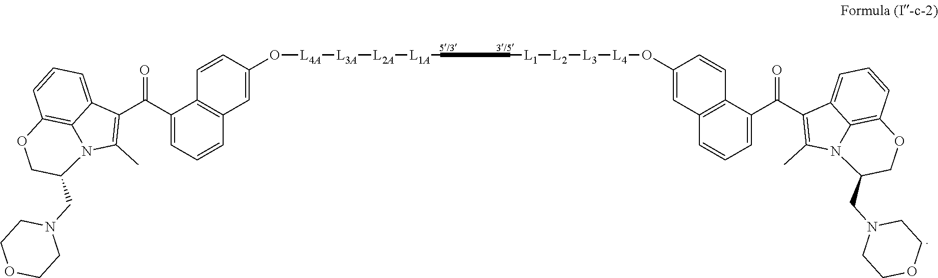

12. A compound, or a stereoisomer, tautomer, prodrug, or salt thereof, comprising the structure of Formula (I): 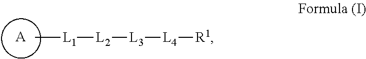
wherein 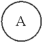
is a cannabinoid receptor type 1 (CB₁) ligand;
each of L₁, L₂, L₃, and L₄ is independently a linker, a bond, or absent; and
R¹ comprises one or more oligonucleotides, protecting groups, small molecules, proteins, antibodies, and/or peptides.

13. The compound, or a stereoisomer, tautomer, prodrug, or salt thereof, of claim 12, wherein the CB₁ ligand is a CB₁ agonist.

14. The compound, or a stereoisomer, tautomer, prodrug, or salt thereof, of claim 12, wherein the CB₁ ligand is a CB₁ antagonist.

15. The compound, or a stereoisomer, tautomer, prodrug, or salt thereof, of claim 12, wherein the CB₁ ligand is a selective ligand.

16. The compound, or a stereoisomer, tautomer, prodrug, or salt thereof, of claim 12, wherein the CB₁ ligand is a non-selective ligand.

17. The compound, or a stereoisomer, tautomer, prodrug, or salt thereof, of any one of claims 12-16, wherein the CB₁ ligand is selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

18. The compound, or a stereoisomer, tautomer, prodrug, or salt thereof, of claim 12, wherein the CB₁ ligand comprises the structure of Formula (II′): 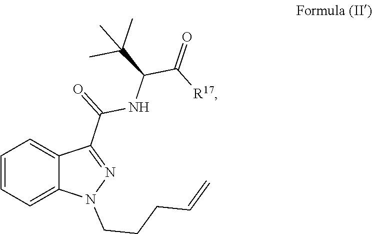
wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D};
—NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, and R^{17D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl;
n17 is 0, 1, 2, 3, or 4; and
v17 is 1 or 2.

19. The compound, or a stereoisomer, tautomer, prodrug, or salt thereof, of claim 18, wherein R¹⁷ is —NR^{17B}R^{17C}, —C(O)R^{17D}, or —C(O)OR^{17D}.

20. The compound, or a stereoisomer, tautomer, prodrug, or salt thereof, of claim 19, wherein R^{17B} and R^{17C} are each independently hydrogen, optionally substituted alkyl, or optionally substituted heteroalkyl.

21. The compound, or a stereoisomer, tautomer, prodrug, or salt thereof, of claim 20, wherein the CB₁ ligand comprises the structure

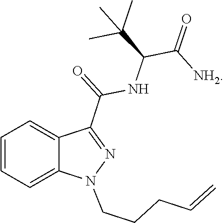 [SMILES: C=CCCCn1nc(C(=O)N[C@H](C(N)=O)C(C)(C)C)c2ccccc21]

22. The compound, or a stereoisomer, tautomer, prodrug, or salt thereof, of claim 12, wherein the compound comprises the structure of Formula (II):

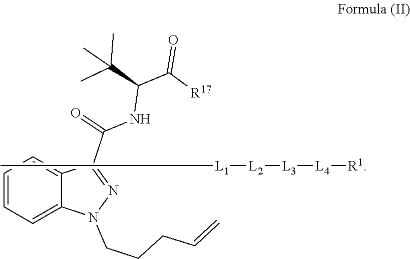

23. The compound, or a stereoisomer, tautomer, prodrug, or salt thereof, of claim 22, wherein the compound comprises the structure of Formula (II-a):

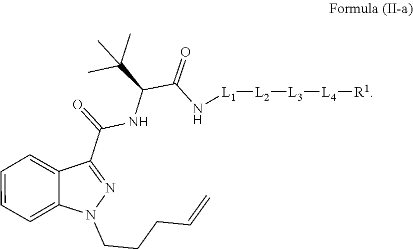

24. The compound, or a stereoisomer, tautomer, prodrug, or salt thereof, of claim 12, wherein the CB₁ ligand comprises the structure 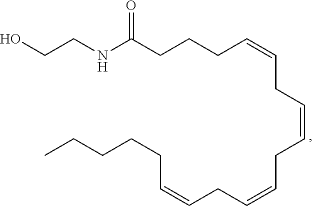 [SMILES: CCCCC/C=C\C/C=C\C/C=C\C/C=C\CCCC(=O)NCCO]
or a derivative thereof.

25. The compound, or a stereoisomer, tautomer, prodrug, or salt thereof, of claim 12, wherein the compound comprises the structure of Formula (III):

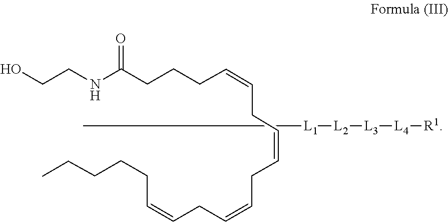

26. The compound, or a stereoisomer, tautomer, prodrug, or salt thereof, of claim 25, wherein the compound comprises the structure of Formula (III-a):

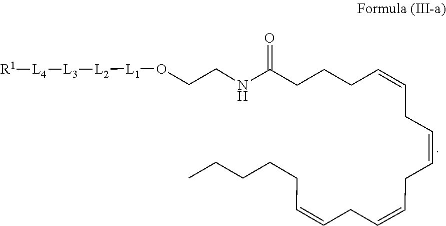

27. A compound, or a stereoisomer, tautomer, prodrug, or salt thereof, comprising the structure of Formula (VIII): 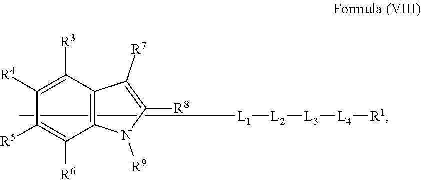
wherein:
each of L₁, L₂, L₃, and L₄ is independently a linker, a bond, or absent;
R¹ comprises one or more oligonucleotides, protecting groups, small molecules, proteins, antibodies, and/or peptides;
R³, R⁴, R⁵, R⁶, and R⁸ are each independently hydrogen, halogen, optionally substituted alkyl, optionally substituted heteroalkyl, optionally substituted cycloalkyl, optionally substituted heterocycloalkyl, optionally substituted aryl, or optionally substituted heteroaryl;
R⁹ is hydrogen, optionally substituted alkyl, or optionally substituted heteroalkyl; or R⁶ and R⁹ substituents may be joined together form an optionally substituted heterocycloalkyl or optionally substituted heteroaryl; and
R⁷ is hydrogen, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D};
—NR^{7B}C(O)OR^{7D}, —NR^{7B}OR^{7D}, optionally substituted alkyl, optionally substituted heteroalkyl, optionally substituted cycloalkyl, optionally substituted heterocycloalkyl, optionally substituted aryl, or optionally substituted heteroaryl;
R^{7A}, R^{7B}, R^{7C}, R^{7D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{7B} and R^{7C} substituents bonded to the same nitrogen atom may optionally be joined to form a substituted or unsubstituted heterocycloalkyl or substituted or unsubstituted heteroaryl;
n7 is 0, 1, 2, 3, or 4; and
v7 is 1 or 2.

28. The compound, or a stereoisomer, tautomer, prodrug, or salt thereof, of claim 27, wherein R⁷ and R⁸ are each independently hydrogen, optionally substituted alkyl, or optionally substituted heteroalkyl.

29. The compound, or a stereoisomer, tautomer, prodrug, or salt thereof, of claim 27 or 28, wherein:
R⁴ is halogen; and
R³, R⁵, and R⁶ are each independently hydrogen.

30. The compound, or a stereoisomer, tautomer, prodrug, or salt thereof, of claim 27 or 28, wherein R³, R⁴, R⁵, and R⁶ are each independently hydrogen.

31. The compound, or a stereoisomer, tautomer, prodrug, or salt thereof, of claim 27 or 28, wherein R⁶ and R⁹ substituents are joined together to form an optionally substituted heterocycloalkyl or optionally substituted heteroaryl.

32. The compound, or a stereoisomer, tautomer, prodrug, or salt thereof, of claim 27 or 28, wherein R⁹ is hydrogen or optionally substituted alkyl.

33. The compound, or a stereoisomer, tautomer, prodrug, or salt thereof, of claim 27, wherein the compound comprises the structure of Formula (VIII-a):

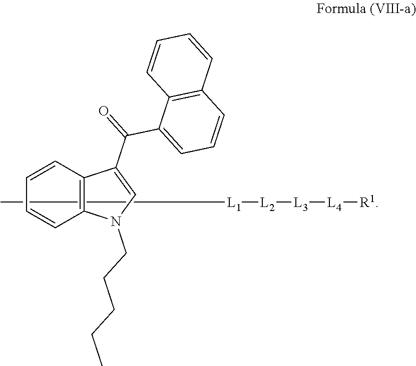

34. The compound, or a stereoisomer, tautomer, prodrug, or salt thereof, of claim 33, wherein the compound comprises the structure of Formula (VIII-a-1):

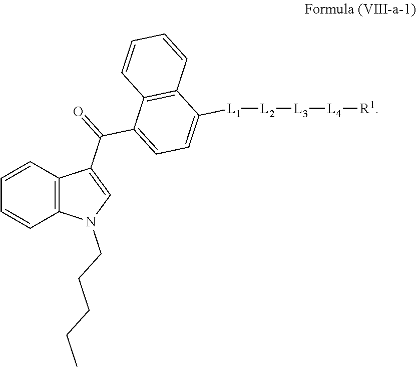

35. The compound, or a stereoisomer, tautomer, prodrug, or salt thereof, of claim 27, wherein the compound comprises the structure of Formula (VIII-a-2):

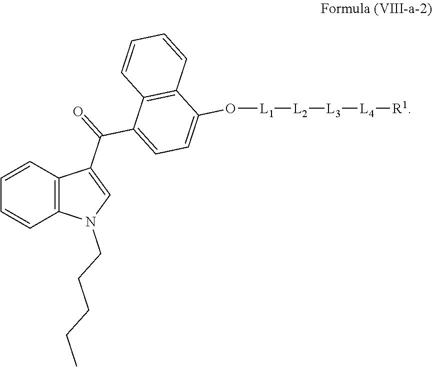

36. The compound, or a stereoisomer, tautomer, prodrug, or salt thereof, of claim 27, wherein the compound comprises the structure of Formula (VIII-b):

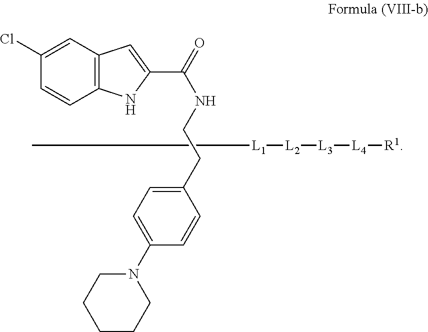

37. The compound, or a stereoisomer, tautomer, prodrug, or salt thereof, of claim 27 or 36, wherein the compound comprises the structure of Formula (VIII-b-1):

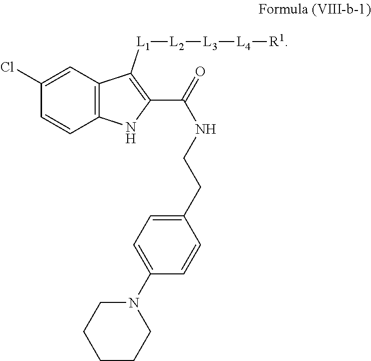

38. The compound, or a stereoisomer, tautomer, prodrug, or salt thereof, of claim 27, wherein the compound comprises the structure of Formula (VIII-c):

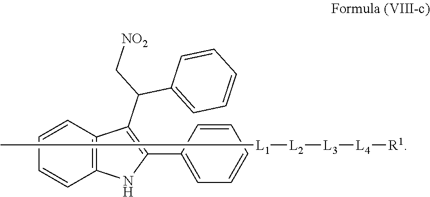

39. The compound, or a stereoisomer, tautomer, prodrug, or salt thereof, of claim 27 or 38, wherein the compound comprises the structure of Formula (VIII-c-1):

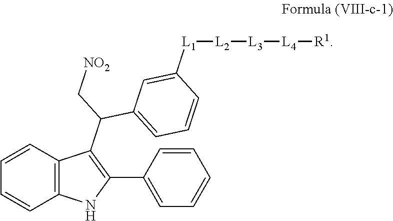

40. The compound, or a stereoisomer, tautomer, prodrug, or salt thereof, of claim 27, wherein the compound comprises the structure of Formula (VIII-c-2):

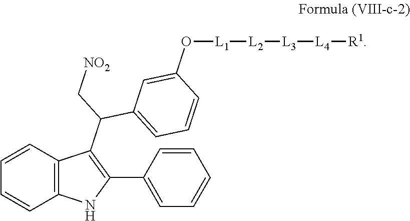

41. The compound, or a stereoisomer, tautomer, prodrug, or salt thereof, of claim 27, wherein the compound comprises the structure of Formula (VIII-d):

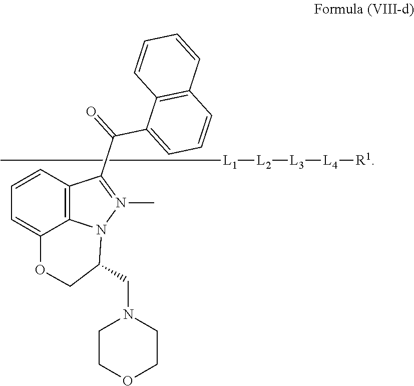

42. The compound, or a stereoisomer, tautomer, prodrug, or salt thereof, of claim 27 or 41, wherein the compound comprises the structure of Formula (VIII-d-1):

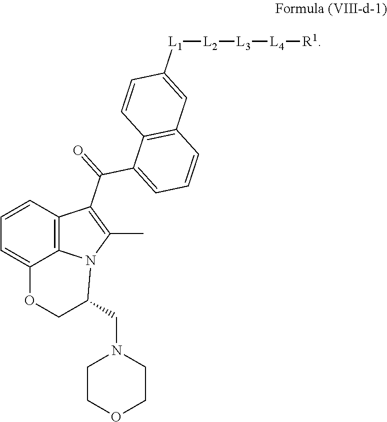

43. The compound, or a stereoisomer, tautomer, prodrug, or salt thereof, of claim 27, wherein the compound comprises the structure of Formula (VIII-d-2):

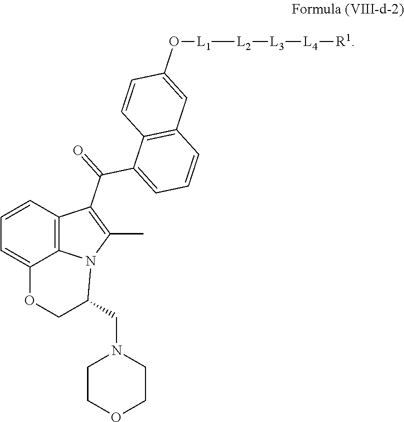

44. The compound, or a stereoisomer, tautomer, prodrug, or salt thereof, of claim 12, comprising the structure of Formula (IX): 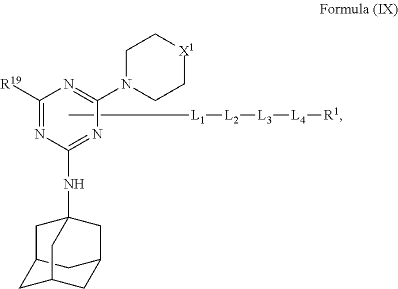
wherein:
each of L₁, L₂, L₃, and L₄ is independently a linker, a bond, or absent;
R¹ comprises one or more oligonucleotides, protecting groups, small molecules, proteins, antibodies, and/or peptides;
X¹ is NR¹⁰ or CR¹¹R¹²;
R¹⁰, R¹¹, and R¹² are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D};
—NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C} substituents bonded to the same nitrogen atom may optionally be joined to form a substituted or unsubstituted heterocycloalkyl or substituted or unsubstituted heteroaryl;
n19 is 0, 1, 2, 3, or 4; and
v19 is 1 or 2.

45. The compound, or a stereoisomer, tautomer, prodrug, or salt thereof, of claim 12 or 44, wherein the compound comprises the structure of Formula (IX-a-1):

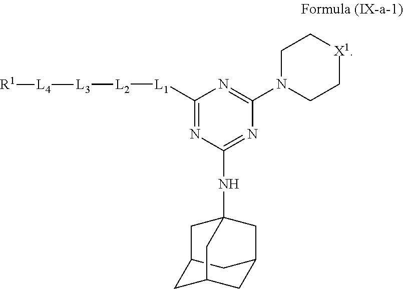

46. The compound, or a stereoisomer, tautomer, prodrug, or salt thereof, of claim 12 or 44, wherein the compound comprises the structure of Formula (IX-a-2):

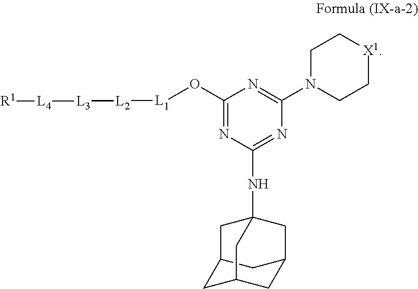

47. The compound, or a stereoisomer, tautomer, prodrug, or salt thereof, of any one of claims 44-46, wherein:
X¹ is NR¹⁰; and
R¹⁰ is hydrogen or optionally substituted alkyl.

48. The compound, or a stereoisomer, tautomer, prodrug, or salt thereof, of claim 47, wherein:
R¹⁰ is hydrogen, —CH₃, or —CH₂CH₂F.

49. The compound, or a stereoisomer, tautomer, prodrug, or salt thereof, of any one of claims 44-46, wherein:
X¹ is CR¹¹R¹²; and
R¹¹ and R¹² are each independently hydrogen or optionally substituted alkyl.

50. The compound, or a stereoisomer, tautomer, prodrug, or salt thereof, of claim 49, wherein:
R¹¹ is hydrogen, —CH₃, or —CH₂CH₂F; and
R¹² is hydrogen.

51. The compound, or a stereoisomer, tautomer, prodrug, or salt thereof, of claim 12, comprising the structure of Formula (X): 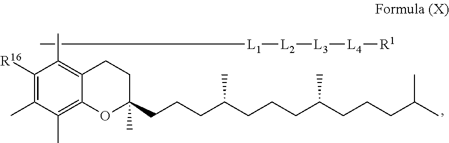
wherein:
each of L₁, L₂, L₃, and L₄ is independently a linker, a bond, or absent;
R¹ comprises one or more oligonucleotides, protecting groups, small molecules, proteins, antibodies, and/or peptides;
R¹⁶ is hydrogen, halogen, —CN, —N₃, —NO₂, —NR^{16B}R^{16C}, —C(O)R^{16D}, —C(O)OR^{16D}, —C(O)NR^{16B}R^{16C}, —OR^{16A}, —NR^{16B}C(O)R^{16D}, optionally substituted alkyl, optionally substituted heteroalkyl, optionally substituted cycloalkyl, optionally substituted heterocycloalkyl, optionally substituted aryl, or optionally substituted heteroaryl; and
R^{16A}, R^{16B}, R^{16C}, and R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{16B} and R^{16C} substituents bonded to the same nitrogen atom may optionally be joined to form a substituted or unsubstituted heterocycloalkyl or substituted or unsubstituted heteroaryl.

52. The compound, or a stereoisomer, tautomer, prodrug, or salt thereof, of claim 51, wherein the compound comprises the structure of Formula (X-a):

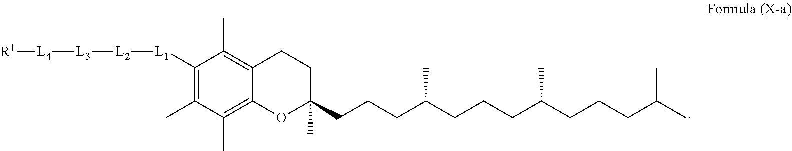

53. The compound, or a stereoisomer, tautomer, prodrug, or salt thereof, of claim 12 or 52, wherein the compound comprises the structure of Formula (X-a-1):

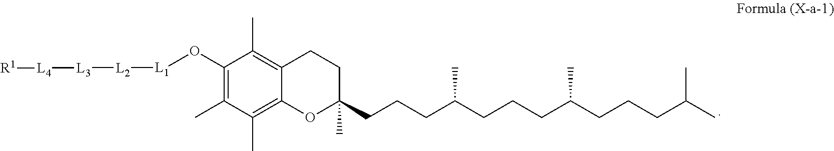

54. The compound, or a stereoisomer, tautomer, prodrug, or salt thereof, of claim 12, wherein the CB₁ ligand comprises the structure: 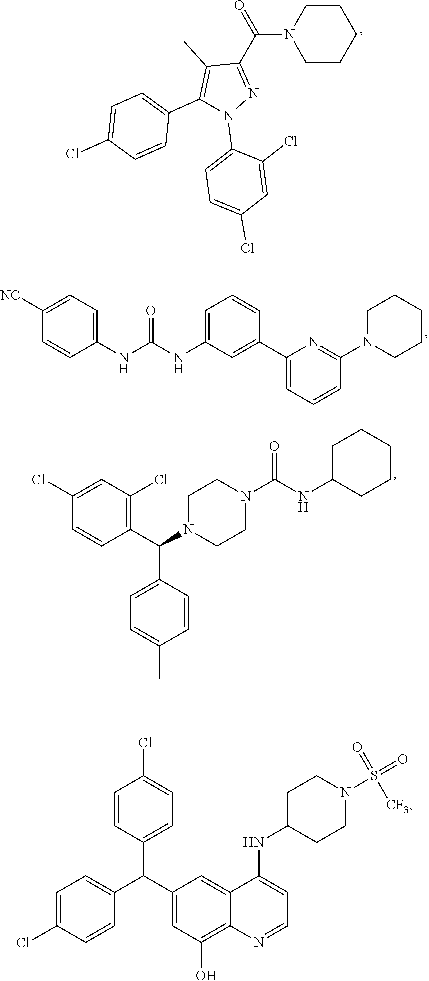
or a derivative thereof.

55. The compound, or a stereoisomer, tautomer, prodrug, or salt thereof, of claim 12, wherein the compound comprises the structure:

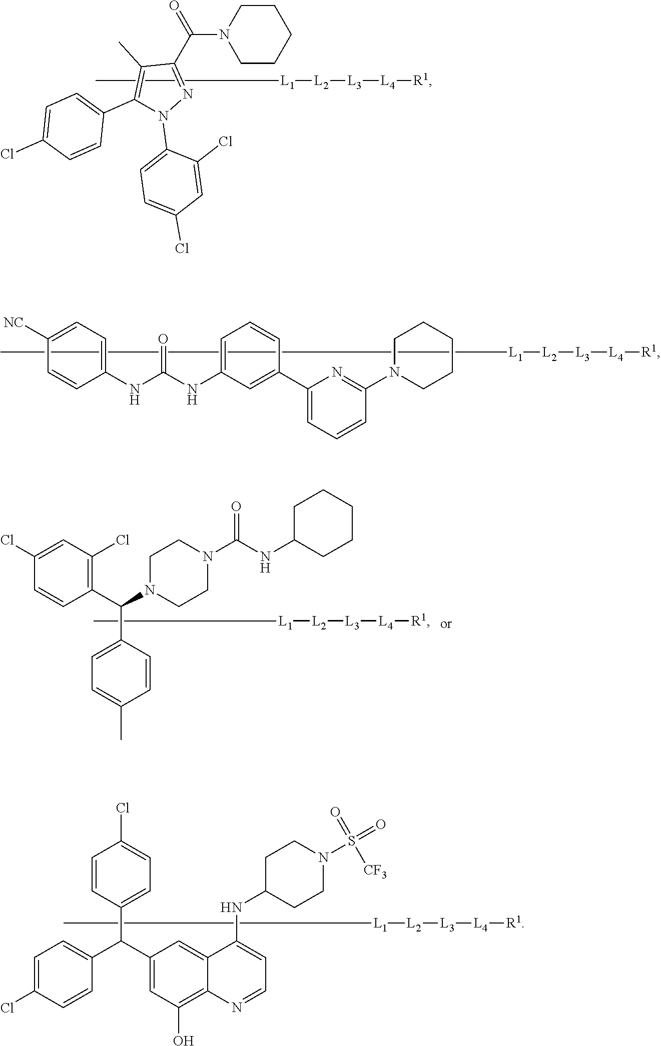

56. The compound, or a stereoisomer, tautomer, prodrug, or salt thereof, of any one of claims 1-55, wherein each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, oxygen, optionally substituted nitrogen, an amide, a phosphodiester bond, or a phosphorothioate bond.

57. The compound, or a stereoisomer, tautomer, prodrug, or salt thereof, of any one of claims 1-56, wherein L₁ is a bond.

58. The compound, or a stereoisomer, tautomer, prodrug, or salt thereof, of any one of claims 1-56, wherein L₁ is oxygen.

59. The compound, or a stereoisomer, tautomer, prodrug, or salt thereof, of any one of claims 1-56, wherein L₁ comprises the structure 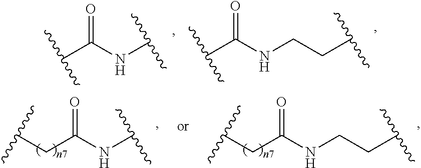
wherein n7 is 0, 1, 2, 3, 4, 5, 6, 7, 8, 9, or 10.

60. The compound, or a stereoisomer, tautomer, prodrug, or salt thereof, of any one of claims 1-59, wherein L₂ is an optionally substituted PEG linker.

61. The compound, or a stereoisomer, tautomer, prodrug, or salt thereof, of claim 60, wherein the PEG linker is 1, 2, 3, 4, 5, 6, 7, 8, 9, or 10 PEG units in length.

62. The compound, or a stereoisomer, tautomer, prodrug, or salt thereof, of any one of claims 1-59, wherein L₂ is an optionally substituted alkyl linker.

63. The compound, or a stereoisomer, tautomer, prodrug, or salt thereof, of claim 62, wherein L₂ comprises the structure

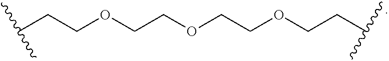 [SMILES: *CCOCCOCCOCC*]

64. The compound, or a stereoisomer, tautomer, prodrug, or salt thereof, of any one of claims 1-63, wherein L₄ is an optionally substituted heteroalkyl linker or a bond.

65. The compound, or a stereoisomer, tautomer, prodrug, or salt thereof, of claim 64, wherein the heteroalkyl linker is substituted with one or more ═O substituents.

66. The compound, or a stereoisomer, tautomer, prodrug, or salt thereof, of claim 64 or 65, wherein L₄ comprises the structure 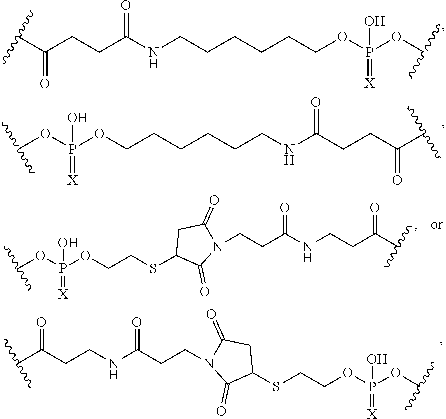
wherein X is O or S.

67. The compound, or a stereoisomer, tautomer, prodrug, or salt thereof, of claim 66, wherein L₄ comprises the structure 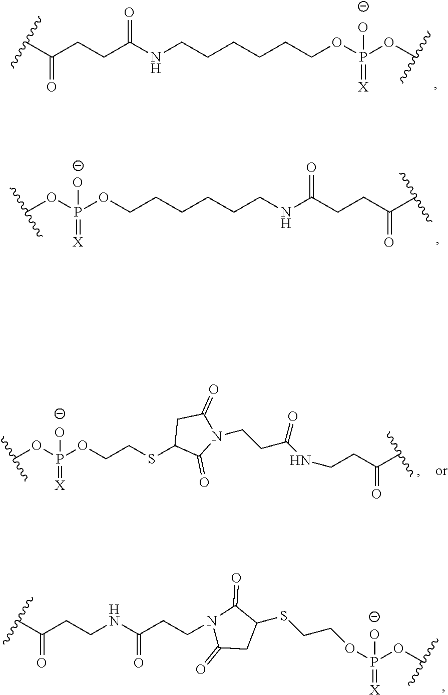
wherein X is O or S.

68. The compound, or a stereoisomer, tautomer, prodrug, or salt thereof, of any one of claims 1-67, wherein L₃ is an optionally substituted heteroaryl linker.

69. The compound, or a stereoisomer, tautomer, prodrug, or salt thereof, of any one of claims 1-63, wherein one of L₃ and L₄ is an optionally substituted phosphodiester bond or an optionally substituted phosphorothioate bond, and the other of L₃ and L₄ is a bond.

70. The compound, or a stereoisomer, tautomer, prodrug, or salt thereof, of any one of claims 1-67, wherein L₃ is an optionally substituted partially unsaturated heteroaryl or optionally substituted partially unsaturated heterocycloalkyl linker.

71. The compound, or a stereoisomer, tautomer, prodrug, or salt thereof, of claim 70, wherein L₃ comprises the structure

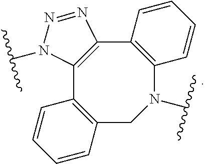 [SMILES: *N1Cc2ccccc2-c2c(nnn2*)-c2ccccc21]

72. The compound, or a stereoisomer, tautomer, prodrug, or salt thereof, of any one of claims 1-71, wherein L₁, L₂, L₃, and L₄ together comprise the structure: 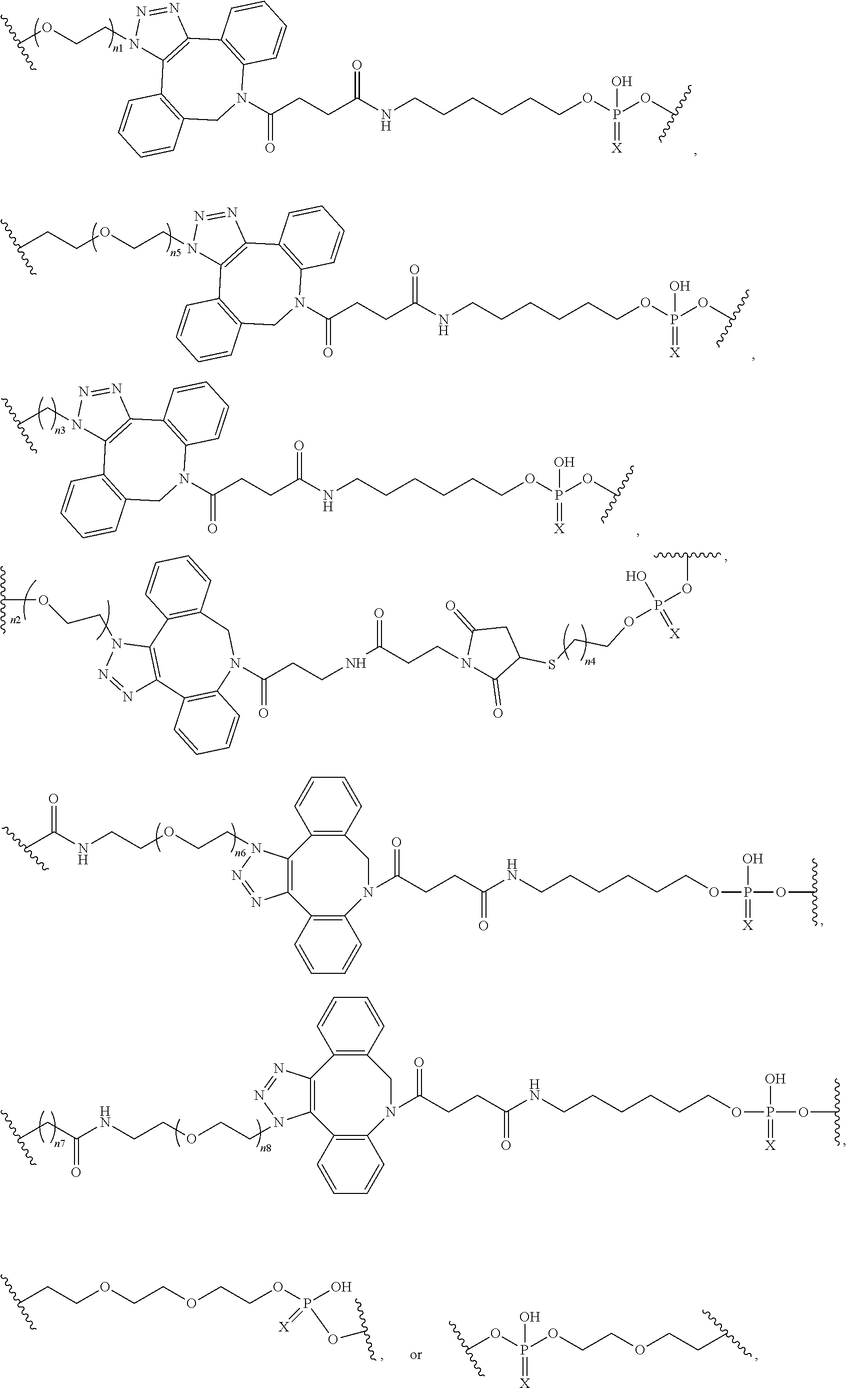
wherein:
X is O or S; and
n1, n2, n4, n5, n6, n7, and n8 are each independently 0, 1, 2, 3, 4, 5, 6, 7, 8, 9, or 10; and
n3 is 0, 1, 2, 3, 4, 5, 6, 7, 8, 9, 10, 11, 12, 13, 14, 15, 16, 17, 18, 19, 20, 21, or 22.

73. The compound, or a stereoisomer, tautomer, prodrug, or salt thereof, of claim 72, wherein L₁, L₂, L₃, and L₄ together comprise the structure: 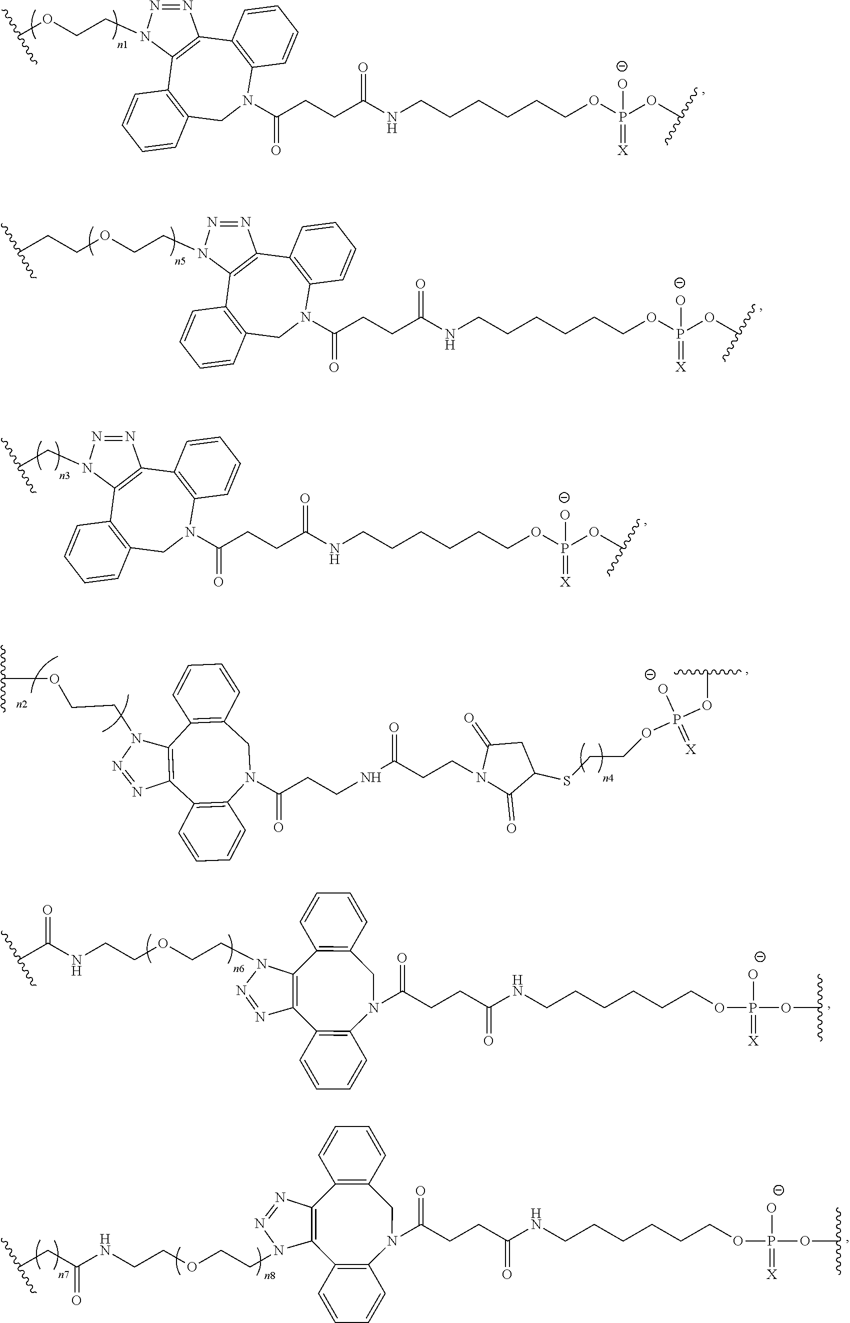 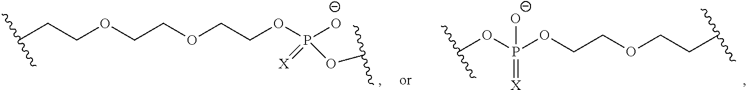
wherein:
X is O or S;
n1, n2, n4, n5, n6, n7, and n8 are each independently 0, 1, 2, 3, 4, 5, 6, 7, 8, 9, 10; and
n3 is 0, 1, 2, 3, 4, 5, 6, 7, 8, 9, 10, 11, 12, 13, 14, 15, 16, 17, 18, 19, 20, 21, or 22.

74. The compound, or a stereoisomer, tautomer, prodrug, or salt thereof, of claim 66, 67, 72, or 73, wherein X is S.

75. The compound, or a stereoisomer, tautomer, prodrug, or salt thereof, of claim 66, 67, 72, or 73, wherein X is O.

76. A compound, or a stereoisomer, tautomer, prodrug, or salt thereof, comprising the structure of Formula (IV′), or a salt thereof: 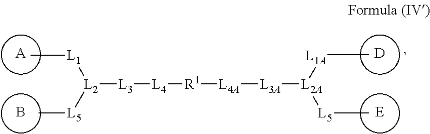
wherein: 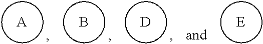
and are each independently a cannabinoid receptor type 1 (CB₁) ligand, or one of 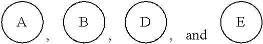
is a CB₁ ligand, and the other of 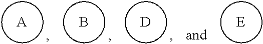
and comprises a lipid or a ligand;
each of L₁, L₂, L₃, L₄, L₅, L_{1A}, L_{2A}, L_{A3}, L_{A4}, and L_{A5} are each independently a linker, a bond, or absent; and
R¹ comprises one or more oligonucleotides, protecting groups, small molecules, proteins, antibodies, and/or peptides.

77. The compound, or a stereoisomer, tautomer, prodrug, or salt thereof, of claim 76, wherein the compound comprises the structure of Formula (IV″): 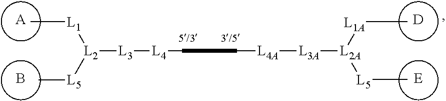
wherein: [symbol]
is an oligonucleotide.

78. The compound, or a stereoisomer, tautomer, prodrug, or salt thereof, of claim 77, wherein the compound comprises the structure of Formula (IV″-a): 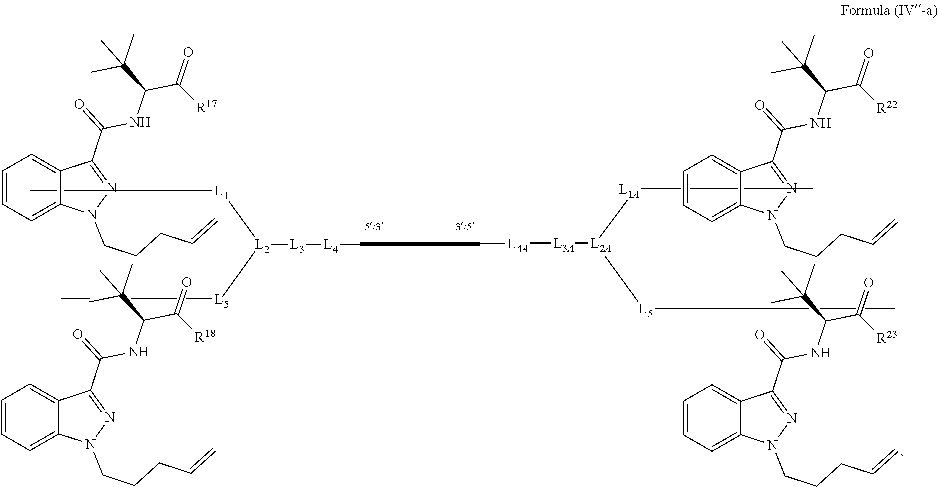
wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D};
—NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R^{18C}, —NHNR^{18B}R^{18C}, —ONR^{18B}R^{18C}, —NHC(O)NHNR^{18B}R^{18C}, —NHC(O)NR^{18B}R^{18C}, —NR^{18B}R^{18C}, —C(O)R^{18D}, —C(O)OR^{18D}, —C(O)NR^{18B}R^{18C}, —OR^{18A}, —NR^{18B}SO₂R^{18A}, —NR^{18B}C(O)R^{18D}, —NR^{18B}C(O)OR^{18D}, —NR^{18B} OR^{18D}, optionally substituted alkyl, optionally substituted heteroalkyl, optionally substituted cycloalkyl, optionally substituted heterocycloalkyl, optionally substituted aryl, or optionally substituted heteroaryl;
R²² is hydrogen, —SOₙ₂₂R^{22A}, —SOᵥ₂₂NR^{22B}R^{22C}, —NHNR^{22B}R^{22C}, —ONR^{22B}R^{22C}, —NHC(O)NHNR^{22B}R^{22C}, —NHC(O)NR^{22B}R^{22C}, —NR^{22B}R^{22C}, —C(O)R^{22D}, —C(O)OR^{22D}, —C(O)NR^{22B}R^{22C}, —OR^{22A}, —NR^{22B}SO₂R^{22A}, —NR^{22B}C(O)R^{22D};
—NR^{22B}C(O)OR^{22D}, —NR^{22B}OR^{22D}, optionally substituted alkyl, optionally substituted heteroalkyl, optionally substituted cycloalkyl, optionally substituted heterocycloalkyl, optionally substituted aryl, or optionally substituted heteroaryl;
R²³ is hydrogen, —SOₙ₂₃R^{23A}, —SOᵥ₂₃NR^{23B}R^{23C}, —NHNR^{23B}R^{23C}, —ONR^{23B}R^{23C}, —NHC(O)NHNR^{23B}R^{23C}, —NHC(O)NR^{23B}R^{23C}, —NR^{23B}R^{23C}, —C(O)R^{23D}, —C(O)OR^{23D}, —C(O)NR^{23B}R^{23C}, —OR^{23A}, —NR^{23B}SO₂R^{23A}, —NR^{23B}C(O)R^{23D};
—NR²³C(O)OR^{23D}, —NR^{23B}OR^{23D}, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, —SOₙ₂₄R^{24A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —NR^{24B}R^{24C}, —C(O)R^{24D}, —C(O)OR^{24D}, —C(O)NR^{24B}R^{24C}, —OR^{24A}, —NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, R^{17D}, R^{18A}, R^{18B}, R^{18C}, R^{18D}, R^{22A}, R^{22B}, R^{22C}, R^{22D}, R^{23A}, R^{23B}, R^{23C}, and R^{23D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl;
n17, n18, n22, and n23 are each independently 0, 1, 2, 3, or 4; and
v17, v18, v22, and v23 are each independently 1 or 2.

79. The compound, or a stereoisomer, tautomer, prodrug, or salt thereof, of claim 78, wherein the compound comprises the structure of Formula (IV″-b):

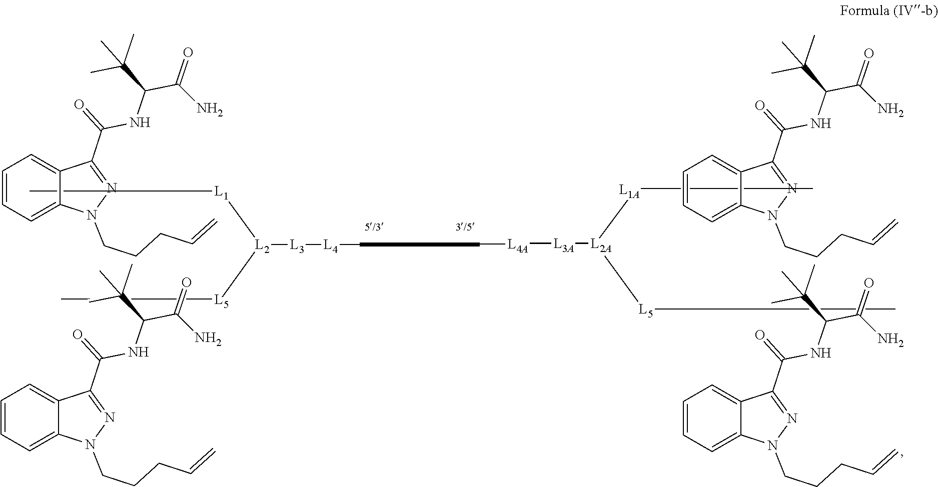

80. The compound, or a stereoisomer, tautomer, prodrug, or salt thereof, of claim 78 or 79, wherein the compound comprises the structure of Formula (IV″-b-1):

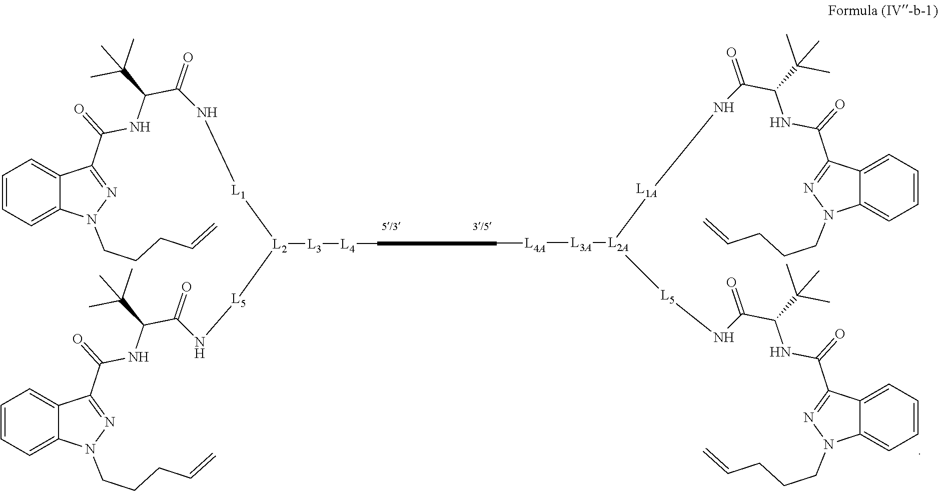

81. A compound, or a stereoisomer, tautomer, prodrug, or salt thereof, comprising the structure of Formula (IV), or a salt thereof: 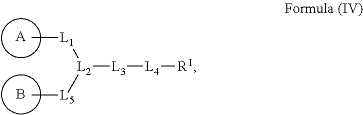
wherein: 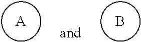
and are each independently a cannabinoid receptor type 1 (CB₁) ligand, or one of 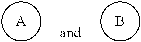
and is a CB₁ ligand, and the other of 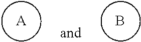
comprises a lipid or a ligand;
each of L₁, L₂, L₃, L₄, and L₅ is independently a linker, a bond, or absent; and
R¹ comprises one or more oligonucleotides, protecting groups, small molecules, proteins, antibodies, and/or peptides.

82. The compound, or a stereoisomer, tautomer, prodrug, or salt thereof, of claim 81, wherein 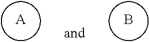
are each independently a CB₁ ligand.

83. The compound, or a stereoisomer, tautomer, prodrug, or salt thereof, of claim 81, wherein one or both of the CB₁ ligands is a CB₁ agonist.

84. The compound, or a stereoisomer, tautomer, prodrug, or salt thereof, of claim 81, wherein one or both of the CB₁ ligands is a CB₁ antagonist.

85. The compound, or a stereoisomer, tautomer, prodrug, or salt thereof, of claim 81, wherein the CB₁ ligand is a selective ligand.

86. The compound, or a stereoisomer, tautomer, prodrug, or salt thereof, of claim 81, wherein the CB₁ ligand is a non-selective ligand.

87. The compound, or a stereoisomer, tautomer, prodrug, or salt thereof, of claim 81, wherein each of the CB₁ ligands is independently selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an ani-CB₁ antibody, and derivatives thereof.

88. The compound, or a stereoisomer, tautomer, prodrug, or salt thereof, of claim 81, wherein each of the CB₁ ligands comprises the structure 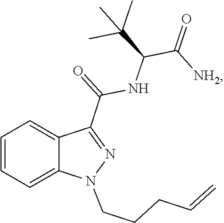 [SMILES: C=CCCCn1nc(C(=O)N[C@H](C(N)=O)C(C)(C)C)c2ccccc21]
or a derivative thereof.

89. The compound, or a stereoisomer, tautomer, prodrug, or salt thereof, of claim 81, wherein the compound comprises the structure of Formula (V): 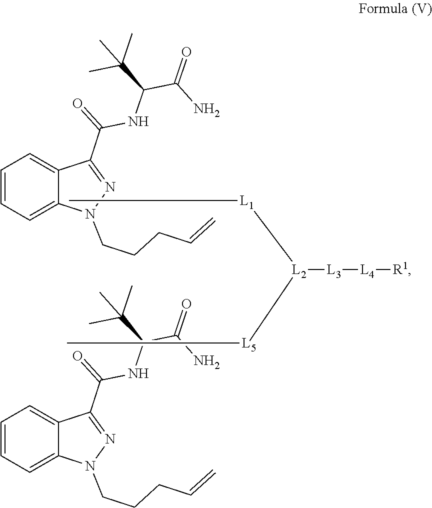
wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R^{18C}, —NHNR^{18B}R^{18C}, —ONR^{18B}R^{18C}, —NHC(O)NHNR^{18B}R^{18C}, —NHC(O)NR¹⁸BR^{18C}, —NR^{18B}R^{18C}, —C(O)R^{18D}, —C(O)OR^{18D}, —C(O)NR^{18B}R^{18C}, —OR^{18A}, —NR^{18B}SO₂R^{18A}, —NR^{18B}C(O)R^{18D}, —NR^{18B}C(O)OR^{18D}, —NR^{18B}OR^{18D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, R^{17D}, R^{18A}, R^{18B}, R^{18C}, and R^{18D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl; R^{17B} and R^{17C} or R^{18B} and R^{18C} substituents bonded to the same nitrogen atom may optionally be joined to form a substituted or unsubstituted heterocycloalkyl or substituted or unsubstituted heteroaryl;
n17 and n18 are each independently 0, 1, 2, 3, or 4; and
v17 and v18 are each independently 1 or 2.

90. The compound, or a stereoisomer, tautomer, prodrug, or salt thereof, of claim 89, wherein the compound comprises the structure of Formula (V-a):

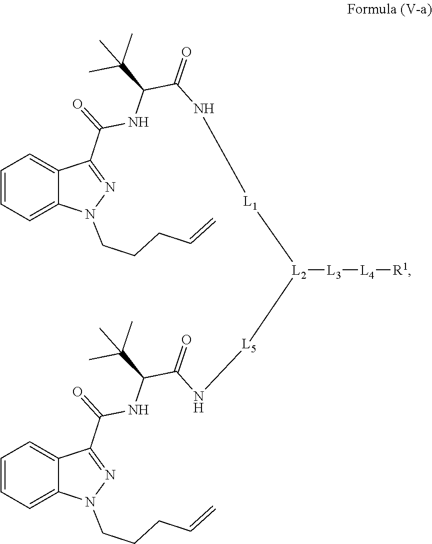

91. The compound, or a stereoisomer, tautomer, prodrug, or salt thereof, of claim 81, wherein one of the CB₁ ligands comprises the structure: 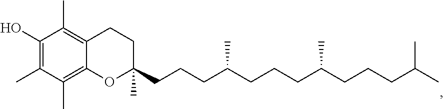 [SMILES: Cc1c(C)c2c(c(C)c1O)CC[C@@](C)(CCC[C@H](C)CCC[C@H](C)CCCC(C)C)O2]
or a derivative thereof, and the other CB₁ ligand comprises the structure: 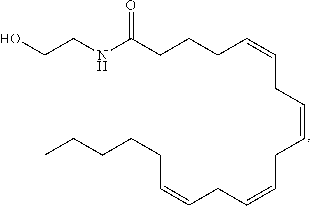 [SMILES: CCCCC/C=C\C/C=C\C/C=C\C/C=C\CCCC(=O)NCCO]
or a derivative thereof.

92. The compound, or a stereoisomer, tautomer, prodrug, or salt thereof, of claim 81, wherein the compound comprises the structure of Formula (IX):

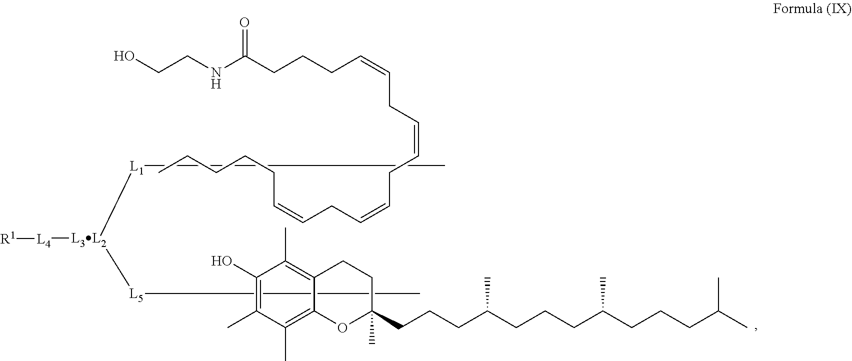

93. The compound, or a stereoisomer, tautomer, prodrug, or salt thereof, of claim 92, wherein the compound comprises the structure of Formula (IX-a):

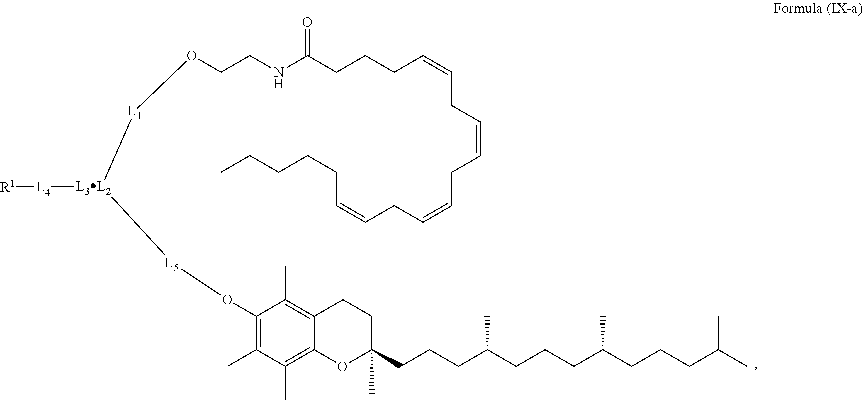

94. The compound, or a stereoisomer, tautomer, prodrug, or salt thereof, of claim 81, wherein each of the CB₁ ligands independently comprises the structure: 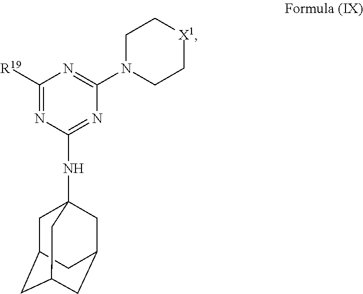
wherein:
X¹ is NR¹⁰ or CR¹¹R¹²;
R¹⁰, R¹¹, and R¹² are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D}, —NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, and R^{19D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{19B} and R^{19C} substituents bonded to the same nitrogen atom may optionally be joined to form a substituted or unsubstituted heterocycloalkyl or substituted or unsubstituted heteroaryl;
n19 is 0, 1, 2, 3, or 4; and
v19 is 1 or 2.

95. The compound, or a stereoisomer, tautomer, prodrug, or salt thereof, of claim 81, wherein the compound comprises the structure of Formula (XI): 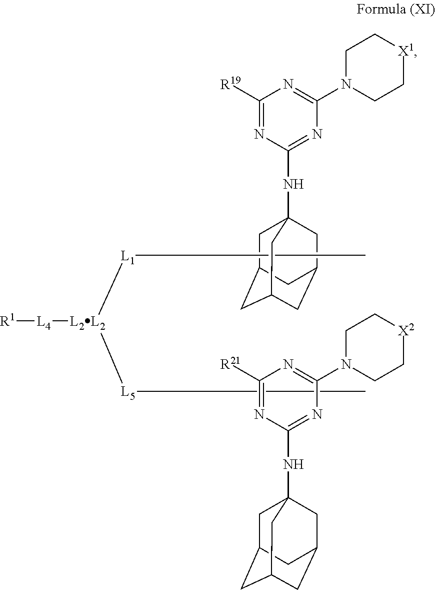
X¹ is independently NR¹⁰ or CR¹¹R¹²;
X² is NR¹³ or CR¹⁴R¹⁵;
R¹⁰, R¹¹, R¹², R¹³, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R^{19A}, R^{19B}, R^{19C}, R^{19D}, R^{21A}, R^{21B}, R^{21C}, and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{21B} and R^{21C} substituents bonded to the same nitrogen atom may optionally be joined to form a substituted or unsubstituted heterocycloalkyl or substituted or unsubstituted heteroaryl;
n19 and n21 are each independently 0, 1, 2, 3, or 4; and
v19 and v21 are each independently 1 or 2.

96. The compound, or a stereoisomer, tautomer, prodrug, or salt thereof, of claim 95, wherein the compound comprises the structure of Formula (XI-a):

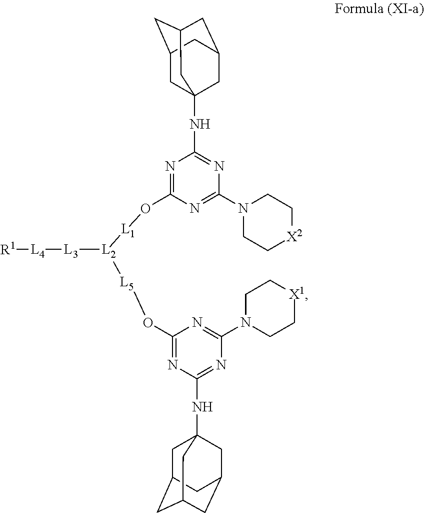

97. The compound, or a stereoisomer, tautomer, prodrug, or salt thereof, of claim 95 or 96, wherein:
X¹ is NR¹⁰;
X² is NR¹³;
R¹⁰ and R¹³ are each independently hydrogen or optionally substituted alkyl.

98. The compound, or a stereoisomer, tautomer, prodrug, or salt thereof, of claim 97, wherein:
R¹⁰ and R¹³ are each independently hydrogen, —CH₃, or —CH₂CH₂F.

99. The compound, or a stereoisomer, tautomer, prodrug, or salt thereof, of claim 95 or 96, wherein:
X¹ is CR¹¹R¹²;
X² is CR¹⁴R¹⁵; and
R¹¹, R¹², R¹⁴, and R¹⁵ are each independently hydrogen or optionally substituted alkyl.

100. The compound, or a stereoisomer, tautomer, prodrug, or salt thereof, of claim 99, wherein:
R¹² and R¹⁵ are each independently hydrogen, —CH₃, or —CH₂CH₂F; and
R¹¹ and R¹⁴ are each independently hydrogen.

101. The compound, or a stereoisomer, tautomer, prodrug, or salt thereof, of any one of claims 81-100, wherein each of L₁, L₂, L₃, L₄, and L₅ is independently absent, a bond, an optionally substituted alkyl linker, an optionally substituted polyethylene glycol (PEG) linker, an optionally substituted heteroalkyl linker, an optionally substituted heteroaryl linker, oxygen, optionally substituted nitrogen, an amide, a phosphodiester bond, or a phosphorothioate bond.

102. The compound, or a stereoisomer, tautomer, prodrug, or salt thereof, of claim 101, wherein L₁ and L₅ are each an optionally substituted PEG linker.

103. The compound, or a stereoisomer, tautomer, prodrug, or salt thereof, of claim 102, wherein L₁ and L₅ are each an optionally substituted PEG linker of 1, 2, 3, 4, 5, 6, 7, 8, 9, or 10 PEG units in length.

104. The compound, or a stereoisomer, tautomer, prodrug, or salt thereof, of any one of claims 81-103, wherein L₂ is an optionally substituted heteroalkyl linker.

105. The compound, or a stereoisomer, tautomer, prodrug, or salt thereof, of claim 104, wherein the heteroalkyl linker is substituted with one or more ═O substituents.

106. The compound, or a stereoisomer, tautomer, prodrug, or salt thereof, of any one of claims 81-105, wherein L₂ comprises the structure

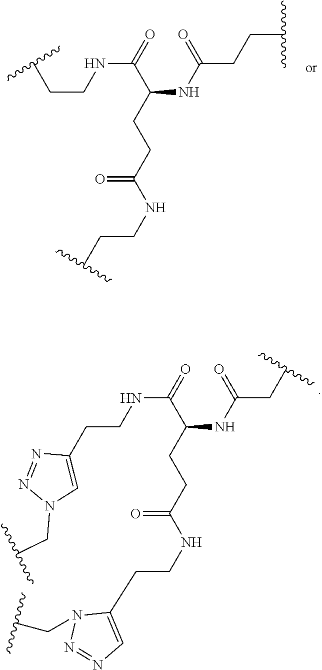 [SMILES: *CCNC(=O)CC[C@H](NC(=O)CC*)C(=O)NCC* ; *CC(=O)N[C@@H](CCC(=O)NCCc1cnnn1C*)C(=O)NCCc1cn(C*)nn1]

107. The compound, or a stereoisomer, tautomer, prodrug, or salt thereof, of any one of claims 81-106, wherein L₃ is an optionally substituted heteroaryl linker.

108. The compound, or a stereoisomer, tautomer, prodrug, or salt thereof, of any one of claims 81-106, wherein L₃ is an optionally substituted partially unsaturated heteroaryl linker or optionally substituted partially unsaturated heterocycloalkyl linker.

109. The compound, or a stereoisomer, tautomer, prodrug, or salt thereof, of claim 108, wherein L₃ comprises the structure

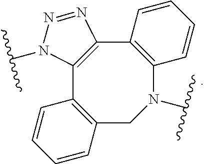 [SMILES: *N1Cc2ccccc2-c2c(nnn2*)-c2ccccc21]

110. The compound, or a stereoisomer, tautomer, prodrug, or salt thereof, of any one of claims 81-109, wherein L₄ is an optionally substituted heteroalkyl linker.

111. The compound, or a stereoisomer, tautomer, prodrug, or salt thereof, of claim 110, wherein the heteroalkyl linker is substituted with one or more ═O substituents.

112. The compound, or a stereoisomer, tautomer, prodrug, or salt thereof, of claim 110 or 111, wherein L₄ comprises the structure 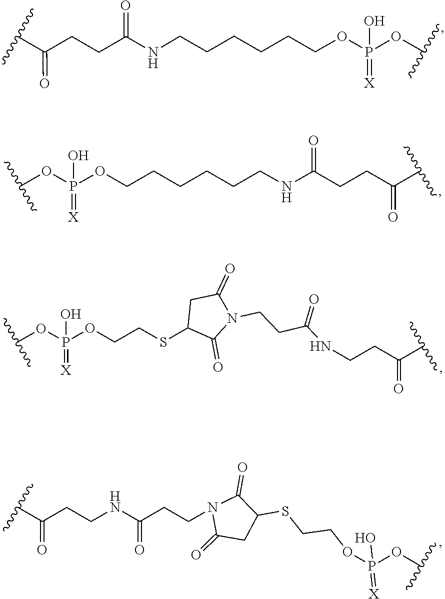
wherein X is O or S.

113. The compound, or a stereoisomer, tautomer, prodrug, or salt thereof, of claim 112, wherein L₄ comprises the structure 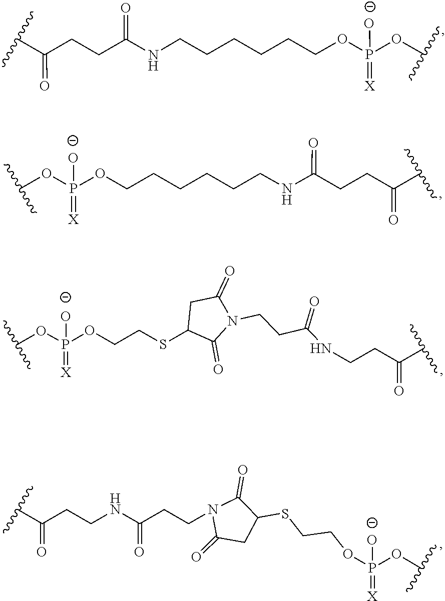
wherein X is O or S.

114. The compound, or a stereoisomer, tautomer, prodrug, or salt thereof, of claim 112 or 113, wherein X is S.

115. The compound, or a stereoisomer, tautomer, prodrug, or salt thereof, of claim 112 or 113, wherein X is O.

116. The compound of any one of claims 81-115, wherein L₁, L₂, L₃, comprise the structure 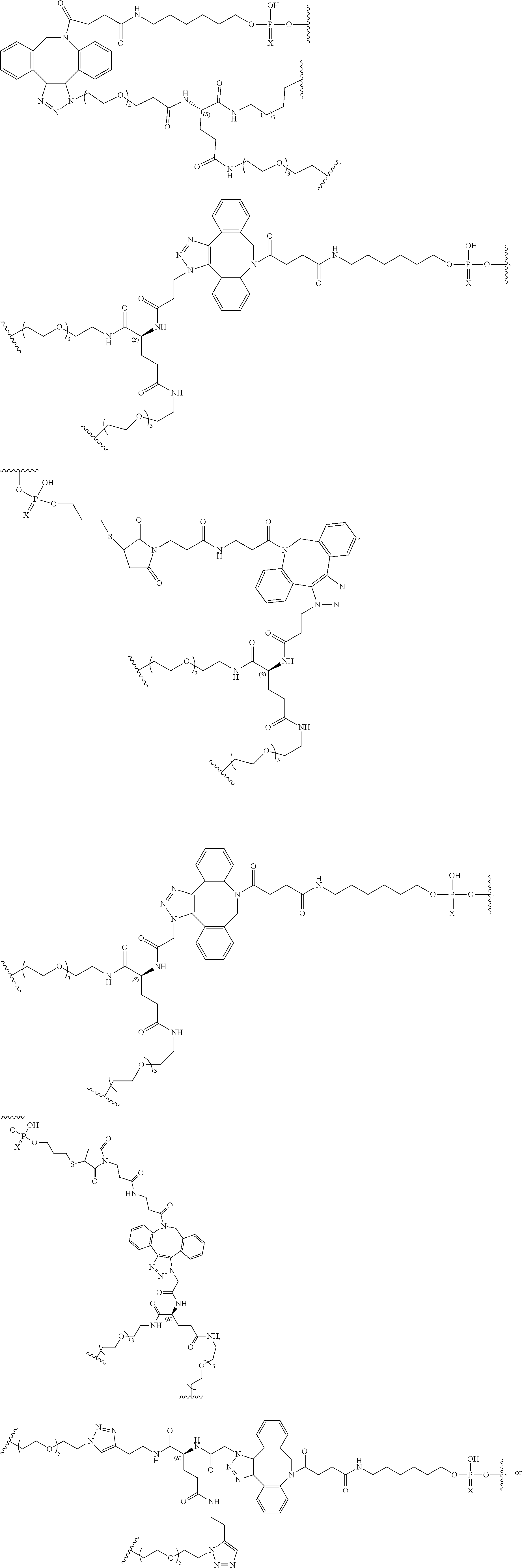 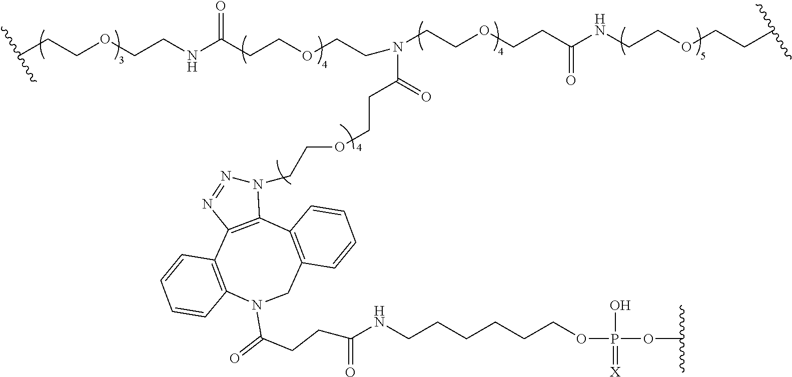
wherein X is O or S.

117. The compound of claim 116, wherein L₁, L₂, L₃, L₄, and L₅ together comprise the structure 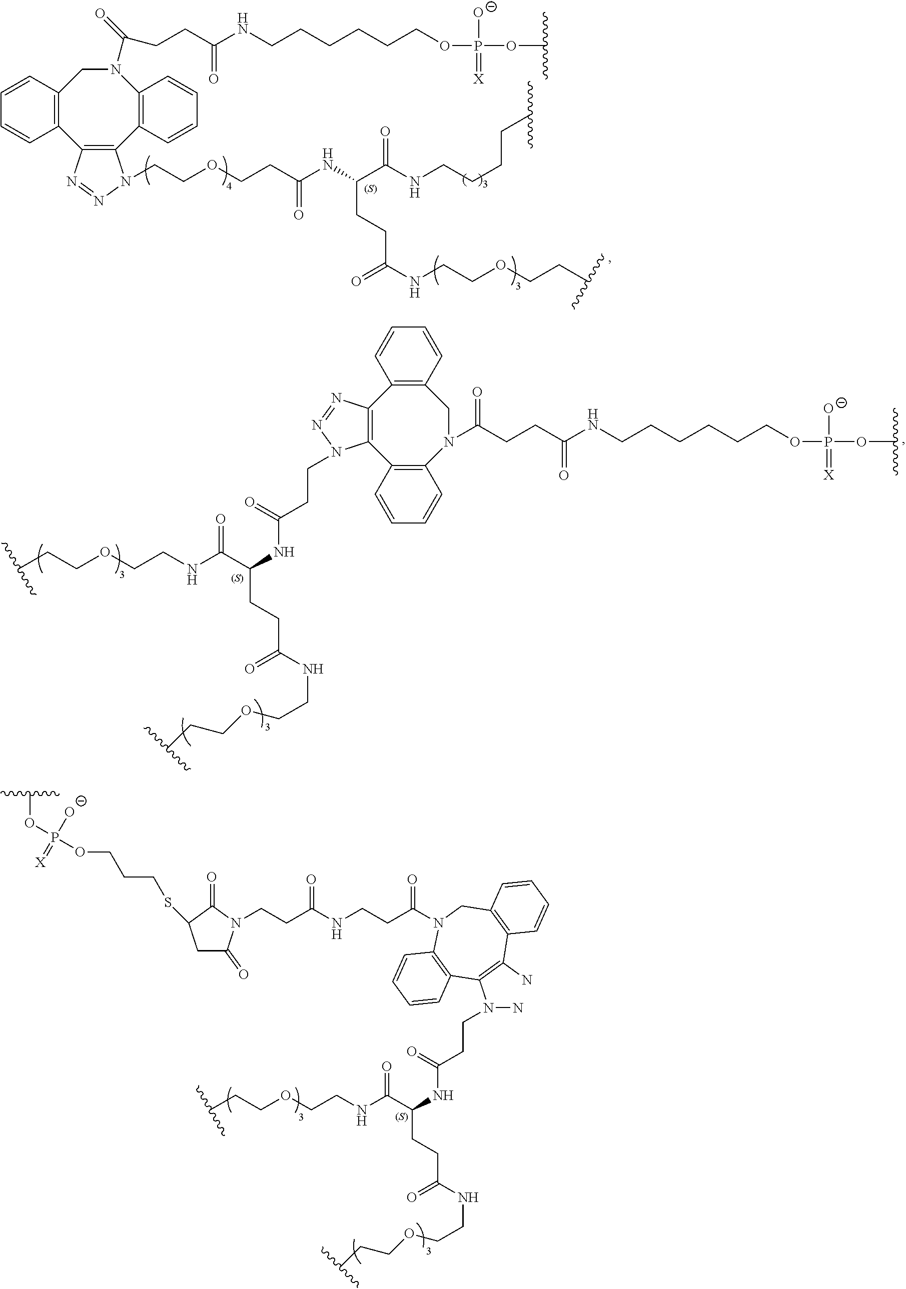 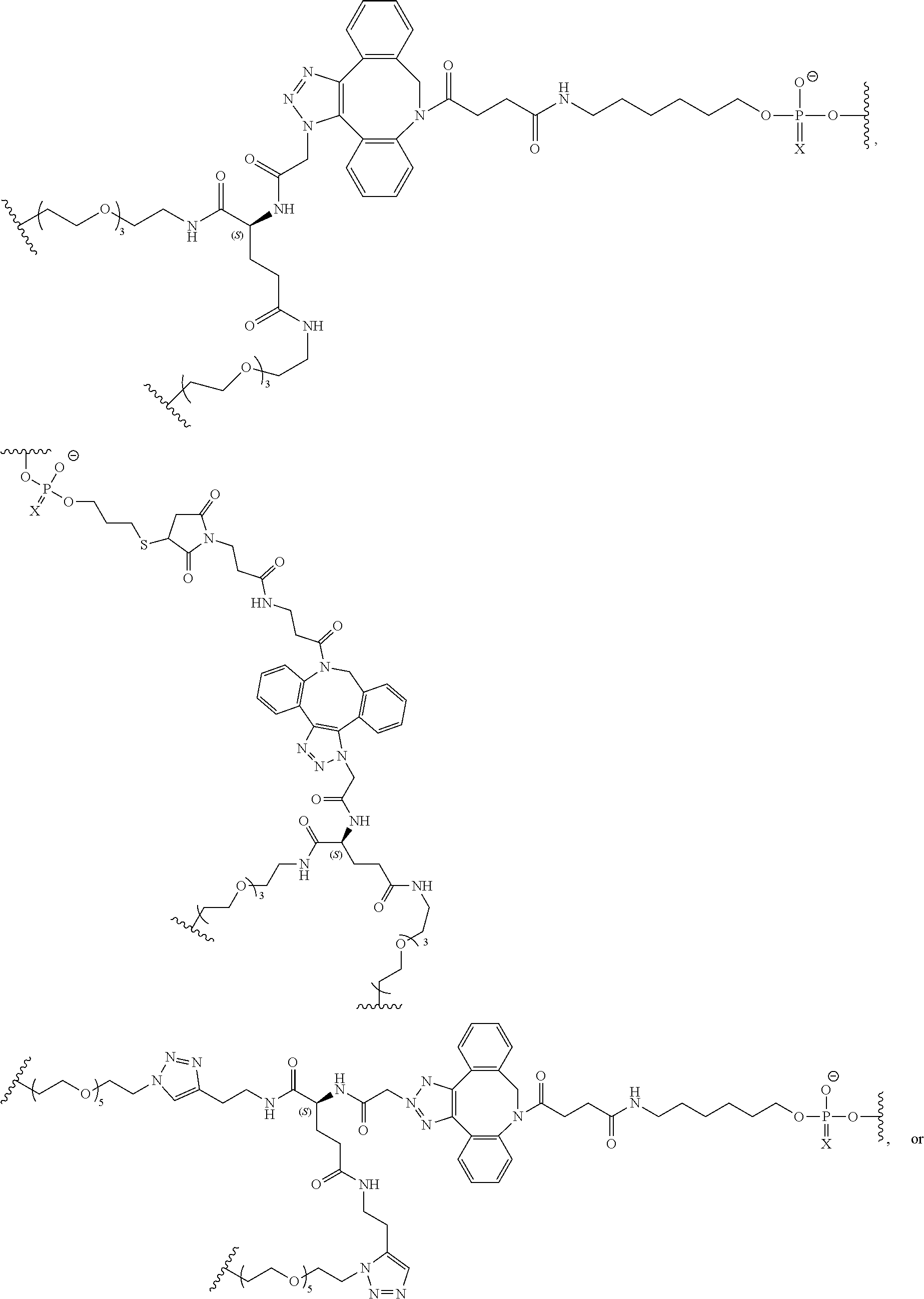 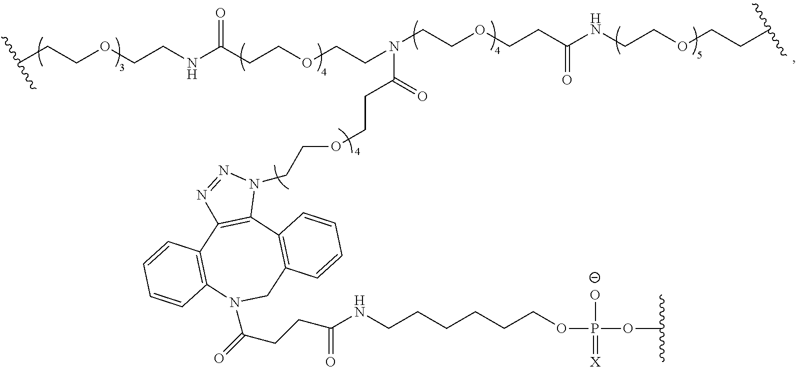
wherein X is O or S.

118. A compound, or a stereoisomer, tautomer, prodrug, or salt thereof, comprising the structure of Formula (VI), or a salt thereof: 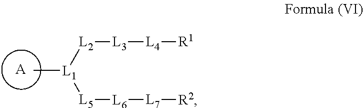
wherein: 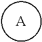
is a cannabinoid receptor type 1 (CB₁) ligand;
each of L₁, L₂, L₃, L₄, L₅, L₆, and L₇ is independently a linker, a bond, or absent; and
R¹ and R² each independently comprise one or more oligonucleotides, protecting groups, small molecules, proteins, antibodies, and/or peptides.

119. The compound, or a stereoisomer, tautomer, prodrug, or salt thereof, of claim 118, wherein the CB₁ ligand is a CB₁ agonist.

120. The compound, or a stereoisomer, tautomer, prodrug, or salt thereof, of claim 118, wherein the CB₁ ligand is a CB₁ antagonist.

121. The compound, or a stereoisomer, tautomer, prodrug, or salt thereof, of claim 118, wherein the CB₁ ligand is selected from the group consisting of minocycline, dronabinol, epigallocatechin, epicatechin, kavain, yangonin, oleamide, N-arachidonoyl dopamine, cannabinol, HU-210, 11-hydroxy-THC, levonantradol, 2-arachidonyl glyceryl ether, JWH-073, tetrahydrocannabinol, 2-arachidonoylglycerol, AM-2201, CP 55,940, JWH-018, WIN 55,212-2, GAT228, cannabigerol, ibipinabant, otenabant, tetrahydrocannabivarin, virodhamine, rimonabant, taranabant, lipoxin A4, ZCZ-011, pregnenolone, cannabidiol, fenofibrate, GAT100, PSNCBAM-1, RVD-Hpα, (S)—N-(1-amino-3,3-dimethyl-1-oxobutan-2-yl)-1-(pent-4-en-1-yl)-1H-indazole-3-carboxamide, anandamide, an anti-CB₁ antibody, and derivatives thereof.

122. The compound, or a stereoisomer, tautomer, prodrug, or salt thereof, of claim 118, wherein the CB₁ ligand comprises the structure

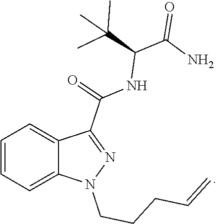 [SMILES: C=CCCCn1nc(C(=O)N[C@H](C(N)=O)C(C)(C)C)c2ccccc21]

123. The compound, or a stereoisomer, tautomer, prodrug, or salt thereof, of claim 118, wherein the compound comprises the structure of Formula (VII): 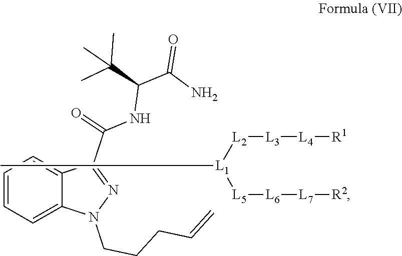
wherein:
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R^{17A}, R^{17B}, R^{17C}, and R^{7D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{17B} and R^{17C} substituents bonded to the same nitrogen atom may optionally be joined to form a substituted or unsubstituted heterocycloalkyl or substituted or unsubstituted heteroaryl
n17 is 0, 1, 2, 3, or 4; and
v17 is 1 or 2.

124. The compound, or a stereoisomer, tautomer, prodrug, or salt thereof, of claim 123, wherein the compound comprises the structure of Formula (VII-a):

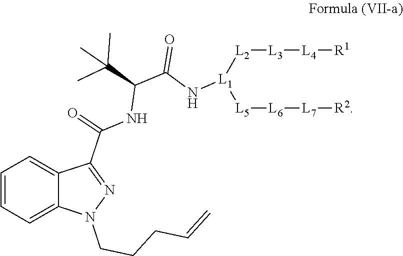

125. The compound, or a stereoisomer, tautomer, prodrug, or salt thereof, of any one of claims 118-124, wherein each of L₁, L₂, L₃, L₄, L₅, L₆, and L₇ is independently absent, a bond, an optionally substituted alkyl linker, an optionally substituted polyethylene glycol (PEG) linker, an optionally substituted heteroalkyl linker, an optionally substituted heteroaryl linker, an optionally substituted saturated or partially unsaturated heterocycloalkyl linker, oxygen, optionally substituted nitrogen, an amide, a phosphodiester bond, or a phosphorothioate bond.

126. The compound, or a stereoisomer, tautomer, prodrug, or salt thereof, of claim 125, wherein L₁ is an optionally substituted PEG linker.

127. The compound, or a stereoisomer, tautomer, prodrug, or salt thereof, of claim 126, wherein L₁ is an optionally substituted PEG linker which is 1, 2, 3, 4, 5, 6, 7, 8, 9, or 10 PEG units in length.

128. The compound, or a stereoisomer, tautomer, prodrug, or salt thereof, of any one of claims 125-127, wherein L₂ and L₅ are each independently an optionally substituted PEG linker.

129. The compound, or a stereoisomer, tautomer, prodrug, or salt thereof, of claim 128, wherein the L₂ and L₅ are each independently an optionally substituted PEG linker three or four PEG units in length.

130. The compound, or a stereoisomer, tautomer, prodrug, or salt thereof, of any one of claims 125-129, wherein L₁, L₂, and L₅ together comprise the structure

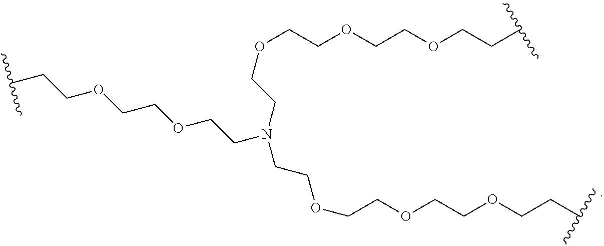 [SMILES: *CCOCCOCCOCCN(CCOCCOCC*)CCOCCOCCOCC*]

131. The compound, or a stereoisomer, tautomer, prodrug, or salt thereof, of any one of claims 125-129, wherein L₃ and L₆ are each independently an optionally substituted heteroaryl linker or an optionally substituted heterocycloalkyl linker.

132. The compound, or a stereoisomer, tautomer, prodrug, or salt thereof, of claim 131, wherein L₃ and L₆ are each independently an optionally substituted partially unsaturated heteroaryl linker or an optionally substituted partially unsaturated heterocycloalkyl linker.

133. The compound, or a stereoisomer, tautomer, prodrug, or salt thereof, of claim 131 or 132, wherein L₃ and L₆ each comprise the structure

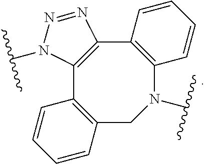 [SMILES: *N1Cc2ccccc2-c2c(nnn2*)-c2ccccc21]

134. The compound, or a stereoisomer, tautomer, prodrug, or salt thereof, of any one of claims 125-133, wherein L₄ and L₇ are each independently an optionally substituted heteroalkyl linker.

135. The compound, or a stereoisomer, tautomer, prodrug, or salt thereof, of claim 134, wherein the heteroalkyl linker is substituted with one or more ═O substituents.

136. The compound, or a stereoisomer, tautomer, prodrug, or salt thereof, of claim 134 or 135, wherein L₄ and L₇ each comprise the structure 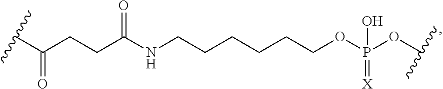
wherein X is O or S.

137. The compound, or a stereoisomer, tautomer, prodrug, or salt thereof, of claim 136, wherein L₄ and L₇ each comprise the structure 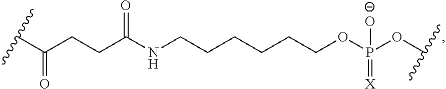
wherein X is O or S.

138. The compound, or a salt thereof, of any one of claims 1-137, wherein R¹ comprises an oligonucleotide.

139. The compound, or a stereoisomer, tautomer, prodrug, or salt thereof, of claim 138, wherein the oligonucleotide is attached at its 5′ end.

140. The compound, or a stereoisomer, tautomer, prodrug, or salt thereof, of claim 138, wherein the oligonucleotide is attached at its 3′ end.

141. The compound, or a stereoisomer, tautomer, prodrug, or salt thereof, of claim 138, wherein the oligonucleotide is attached at an internal position on the oligonucleotide.

142. The compound, or a stereoisomer, tautomer, prodrug, or salt thereof, of claim 141, wherein the internal position is an internucleoside linkage.

143. The compound, or a stereoisomer, tautomer, prodrug, or salt thereof, of any one of claims 1-142, wherein R¹ comprises an oligonucleotide conjugated to one or more additional CB₁ ligands.

144. The compound, or a stereoisomer, tautomer, prodrug, or salt thereof, of claim 143, wherein the oligonucleotide is conjugated to two, three, four, five, or more than five additional CB₁ ligands.

145. The compound, or a stereoisomer, tautomer, prodrug, or salt thereof, of claim 143 or 144, wherein the additional CB₁ ligands are conjugated to the oligonucleotide at the 5′ end of the oligonucleotide, the 3′ end of the oligonucleotide, one or more internal positions on the oligonucleotide, or any combination thereof.

146. The compound of any one of claims 1-145, or a stereoisomer, tautomer, prodrug, or salt thereof, wherein the oligonucleotide is a modified oligonucleotide.

147. The compound, or a salt thereof, of any one of claims 1-146, wherein L₁, L₂, L₃, and L₄ together comprise the structure: 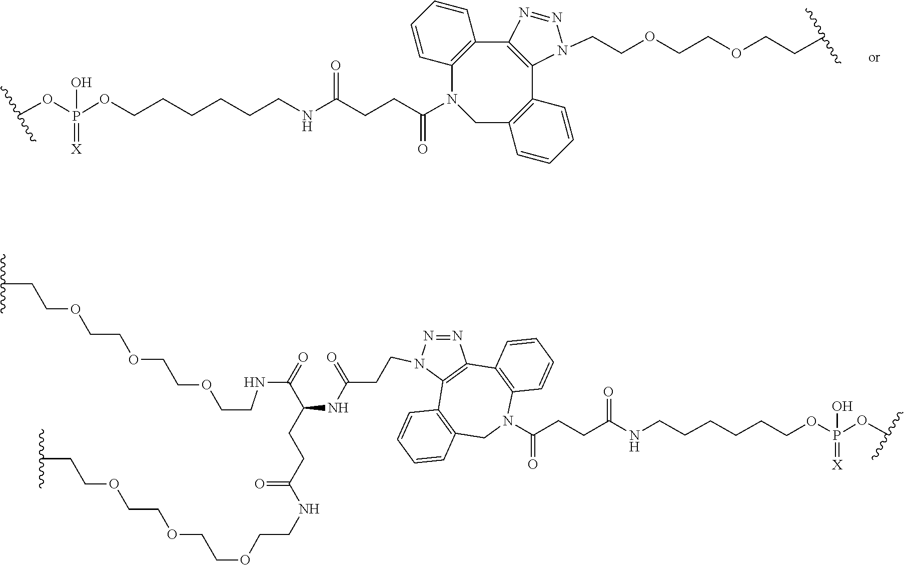
wherein X is O or S.

148. The compound, or a stereoisomer, tautomer, prodrug, or salt thereof, of claim 147, wherein L₁, L₂, L₃, and L₄ together comprise the structure: 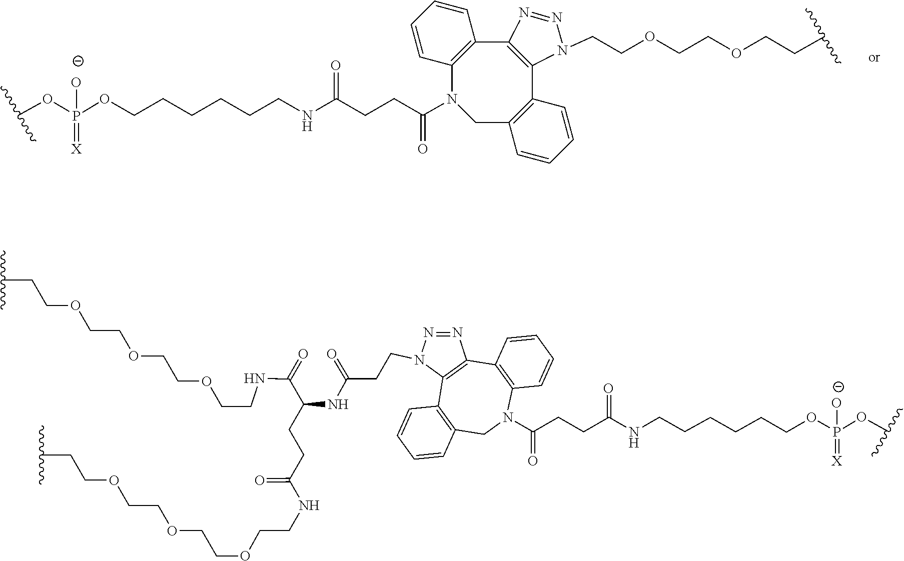
wherein X is O or S.

149. The compound, or a salt thereof, of any one of claims 118-146, wherein L₁, L₂, L₃, L₄, L₅, L₆, and L₇ together comprise the structure: 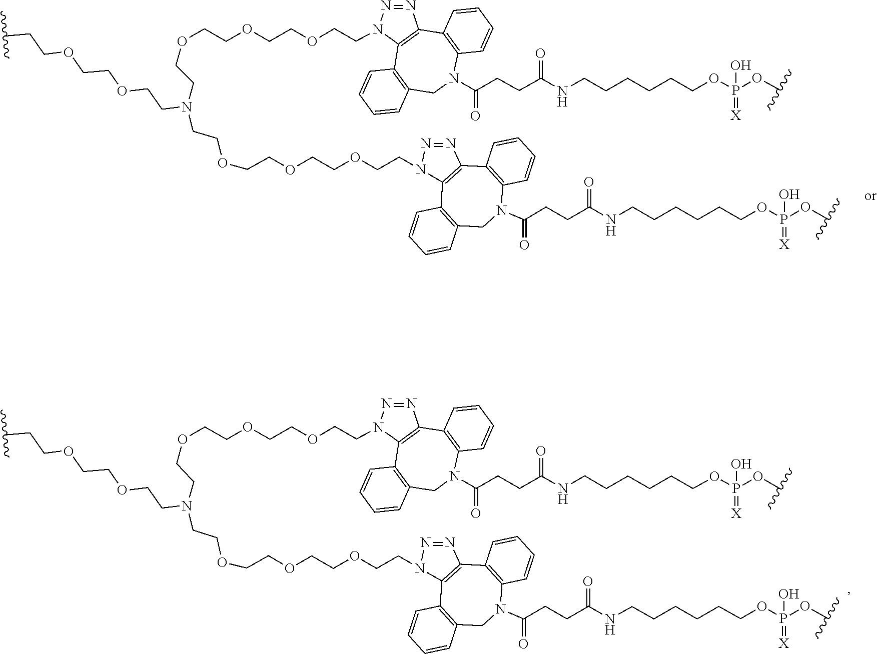
wherein X is O or S.

150. The compound, or a stereoisomer, tautomer, prodrug, or salt thereof, of claim 149, wherein L₁, L₂, L₃, L₄, L₅, L₆, and L₇ together comprise the structure 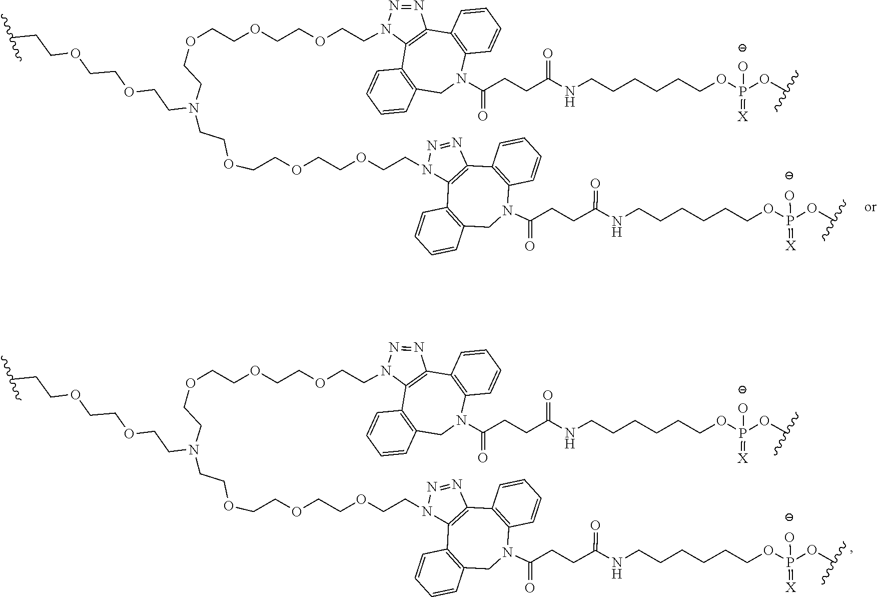
wherein X is O or S.

151. A compound, or a stereoisomer, tautomer, prodrug, or salt thereof, comprising the structure: 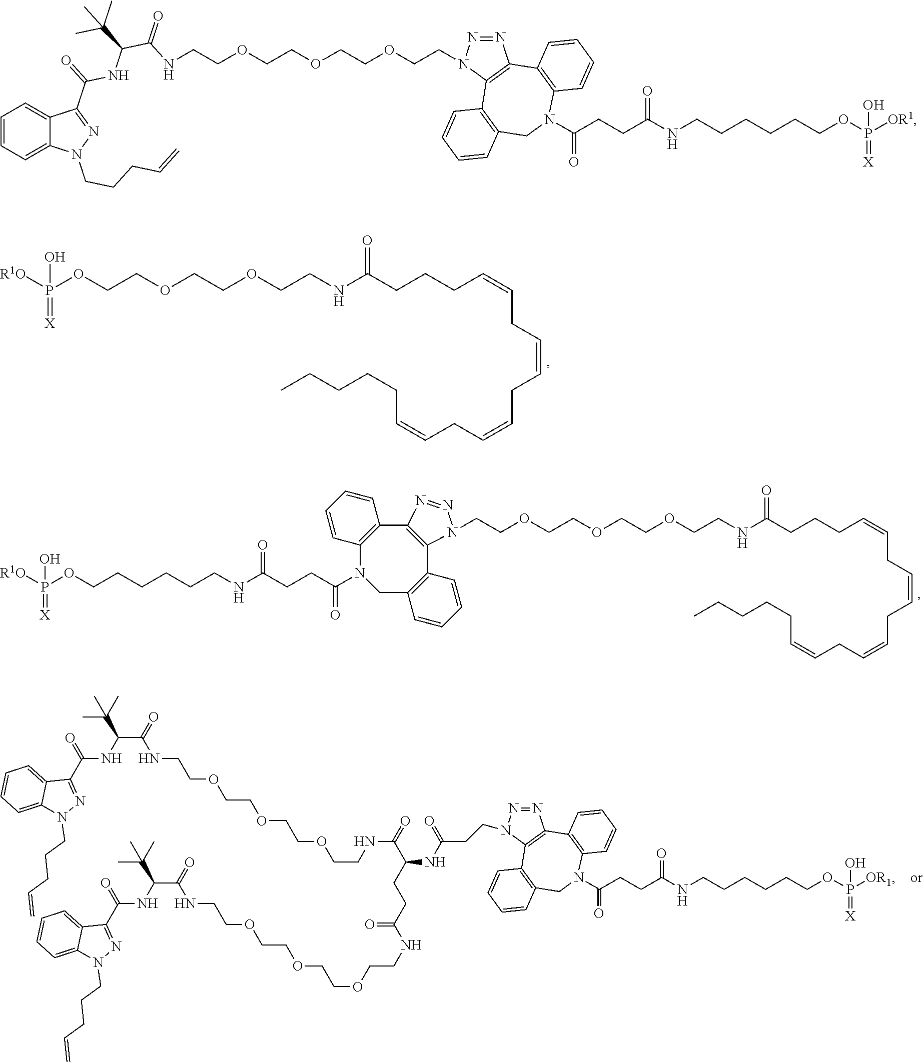 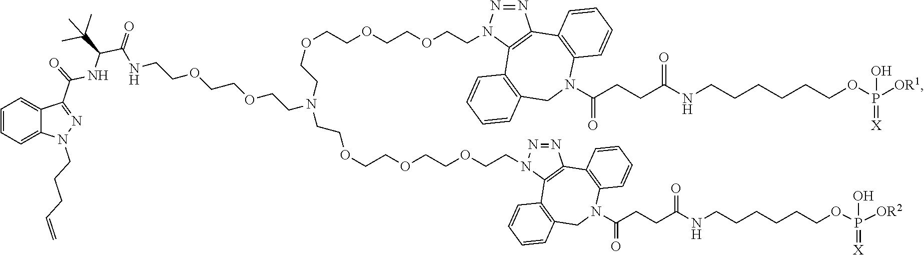
wherein:
R¹ and R² each independently comprise one or more oligonucleotides, protecting groups, small molecules, proteins, antibodies, and/or peptides; and
X is O or S.

152. The compound of claim 151, wherein the compound comprises the structure:

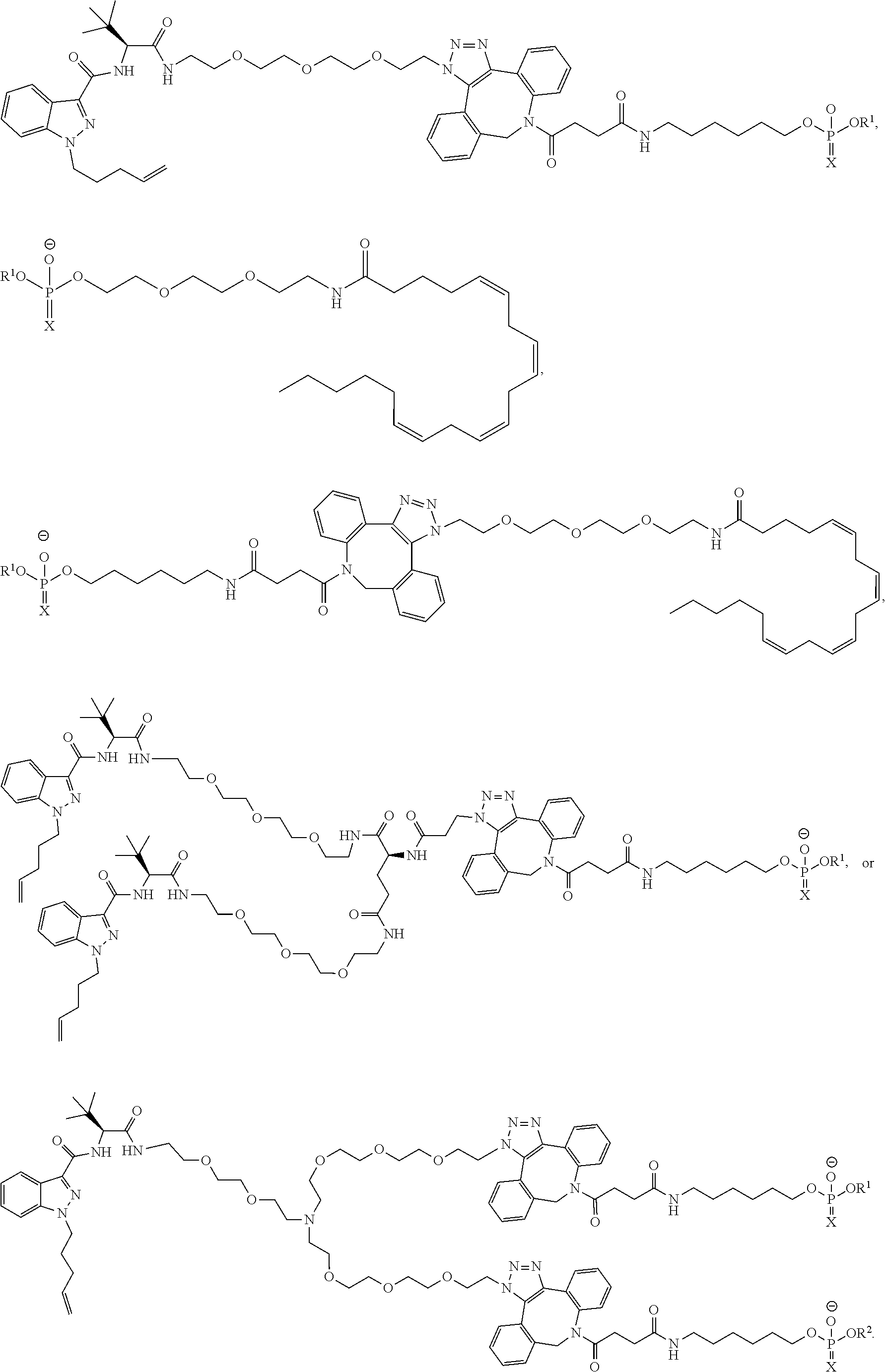

153. A compound, or a stereoisomer, tautomer, prodrug, or salt thereof, comprising the structure: 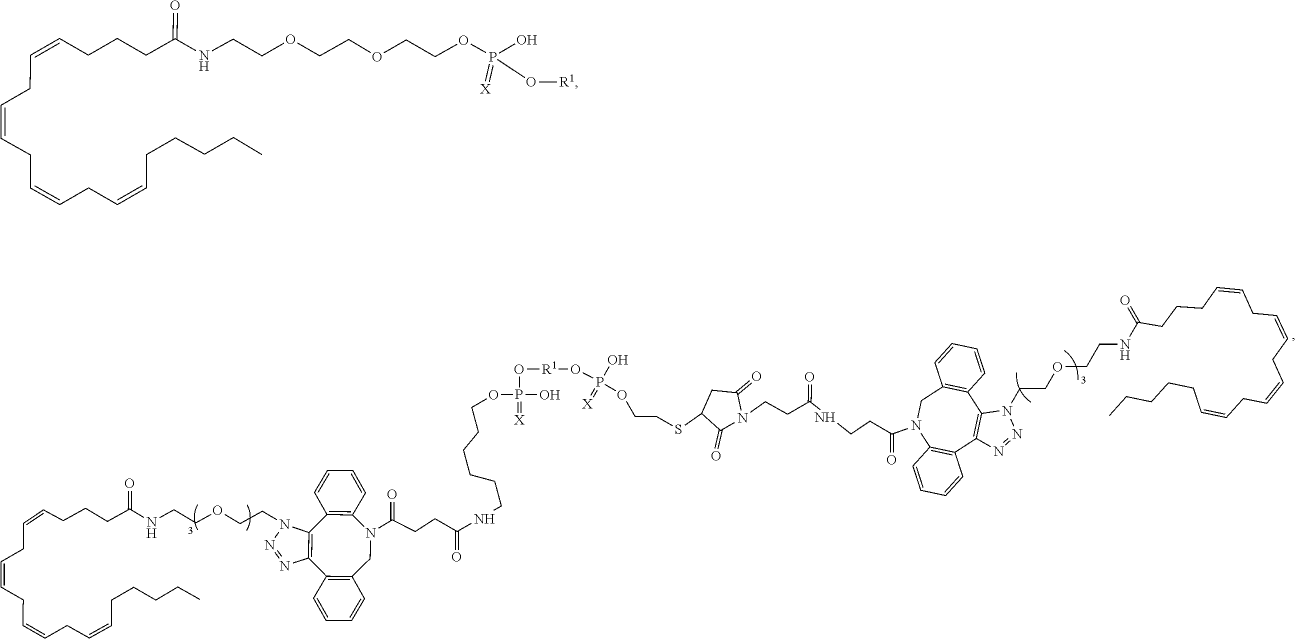 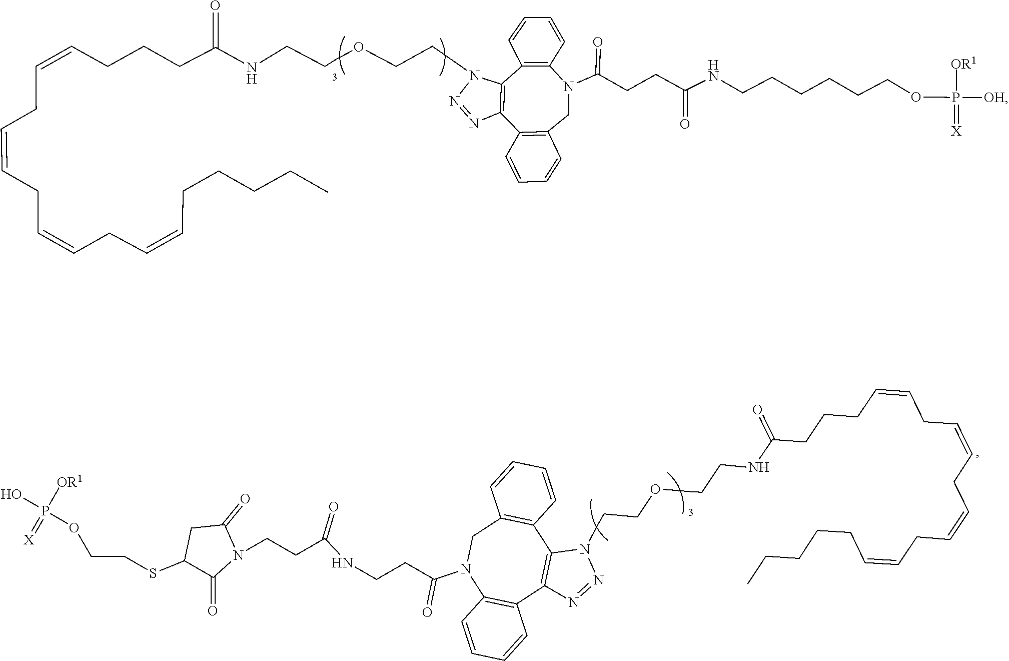 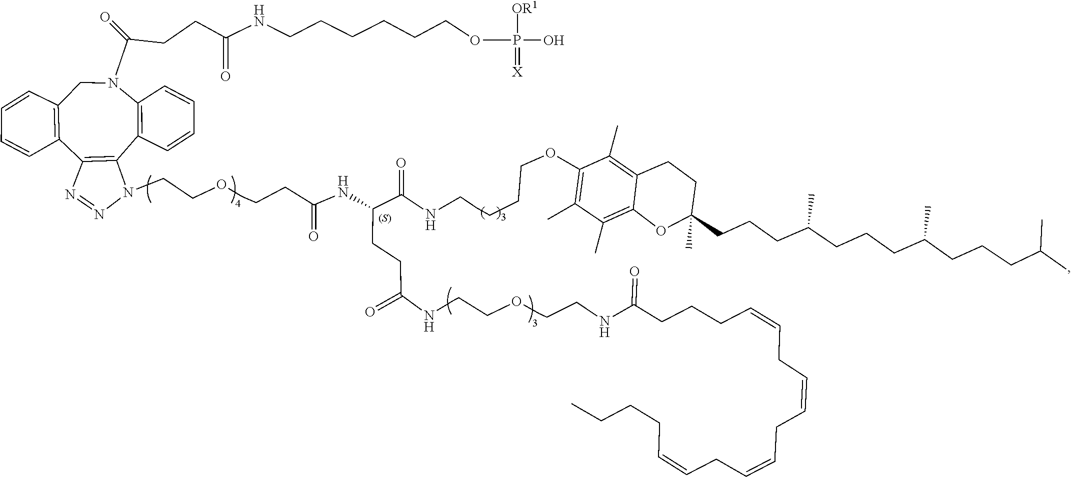 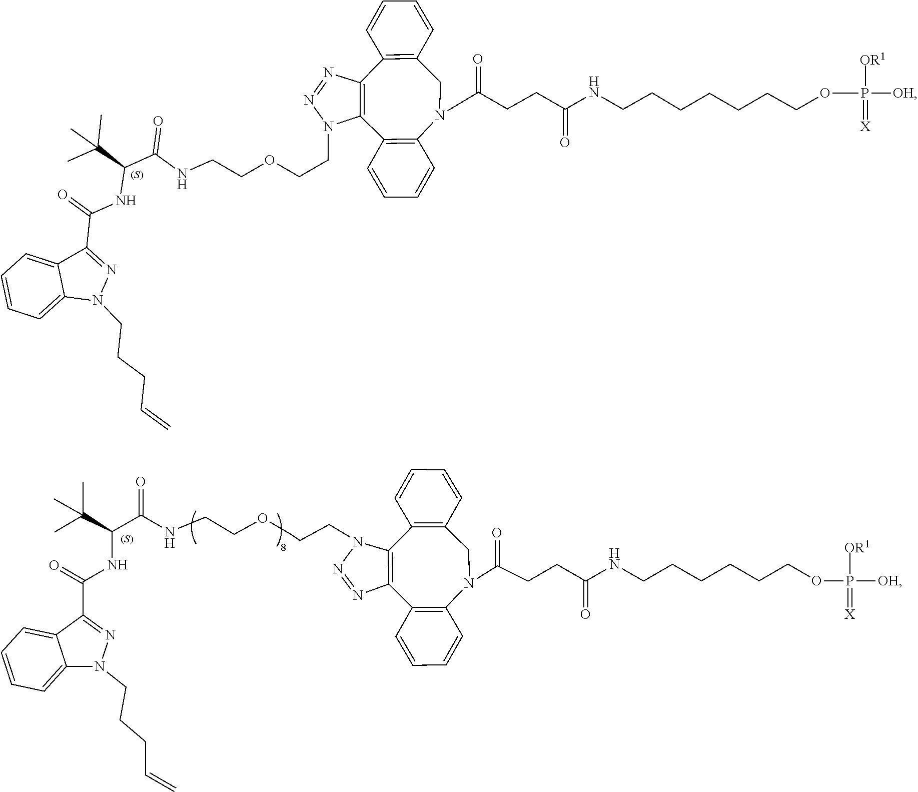 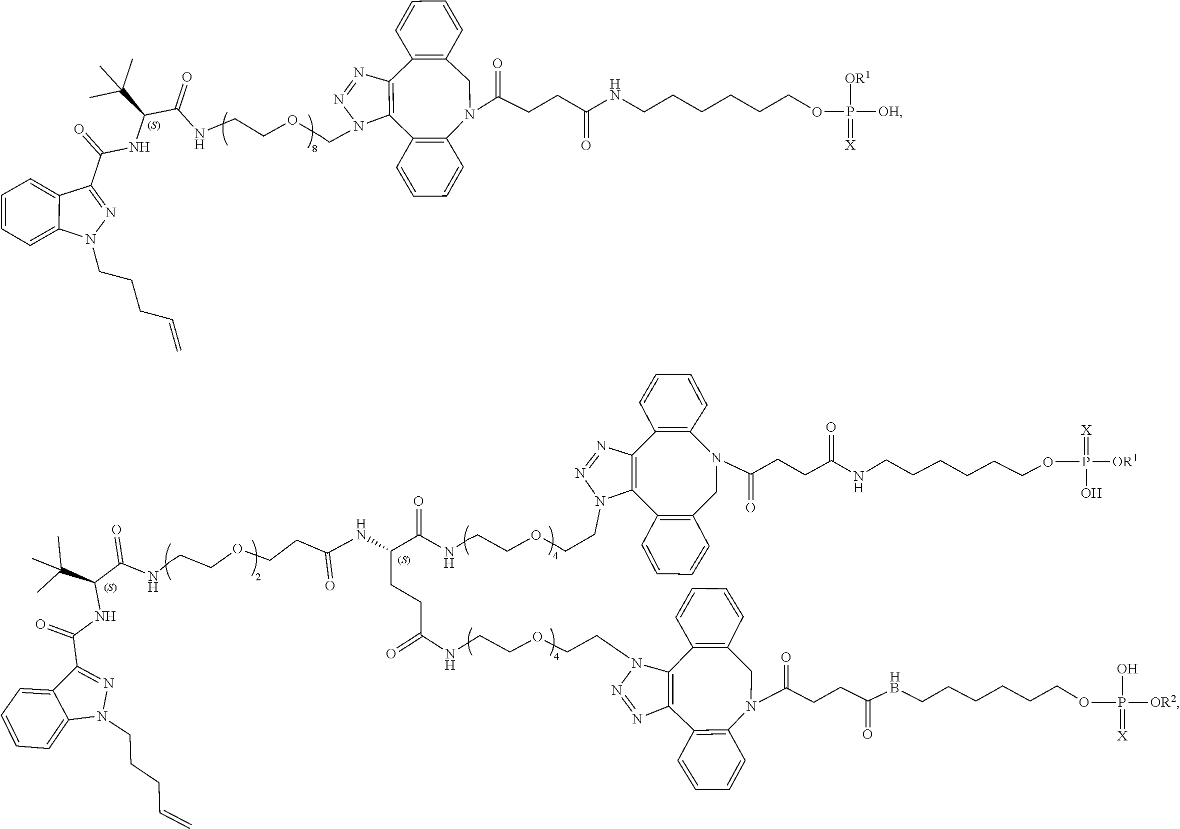 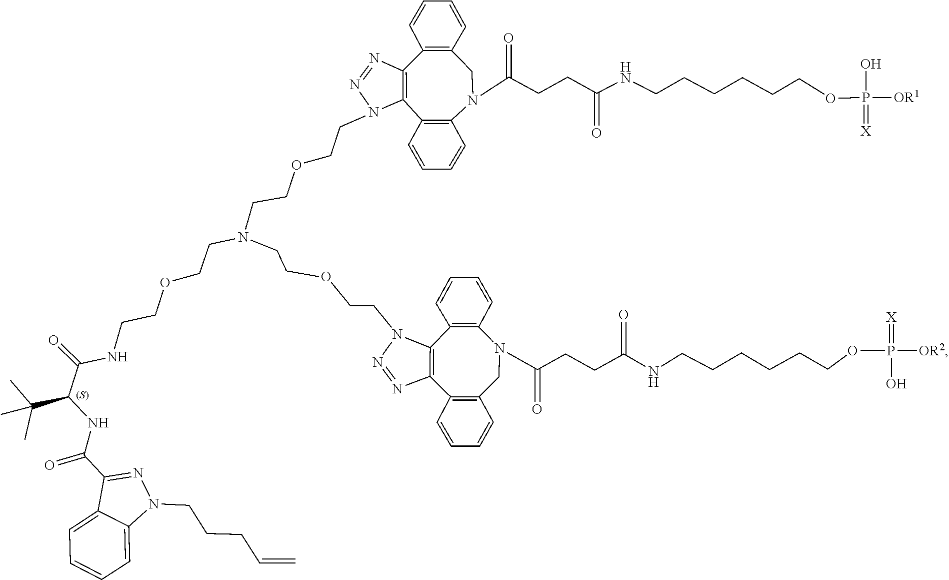 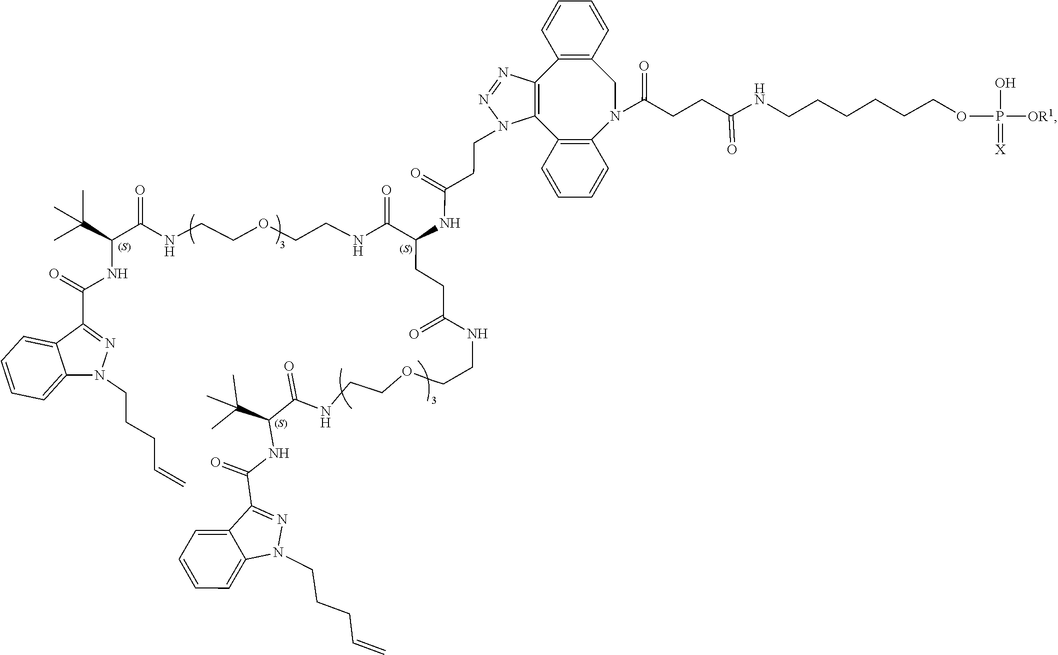 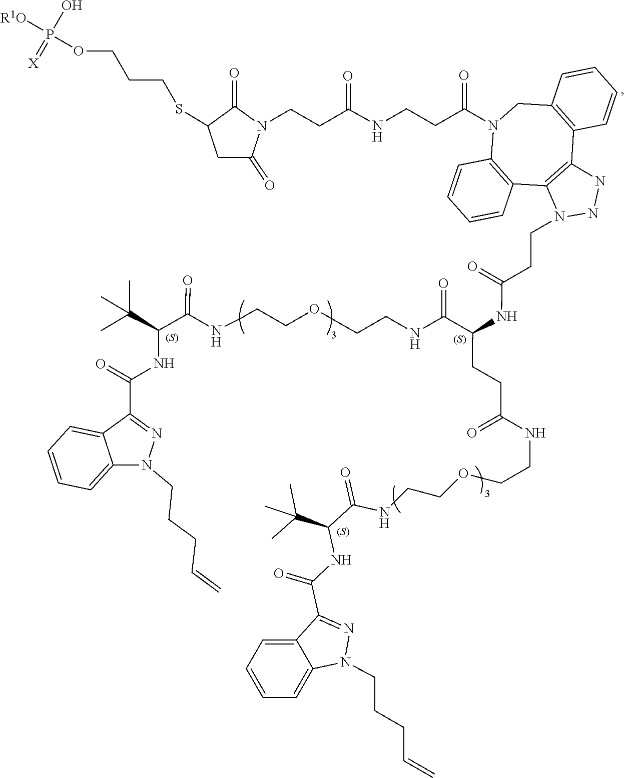 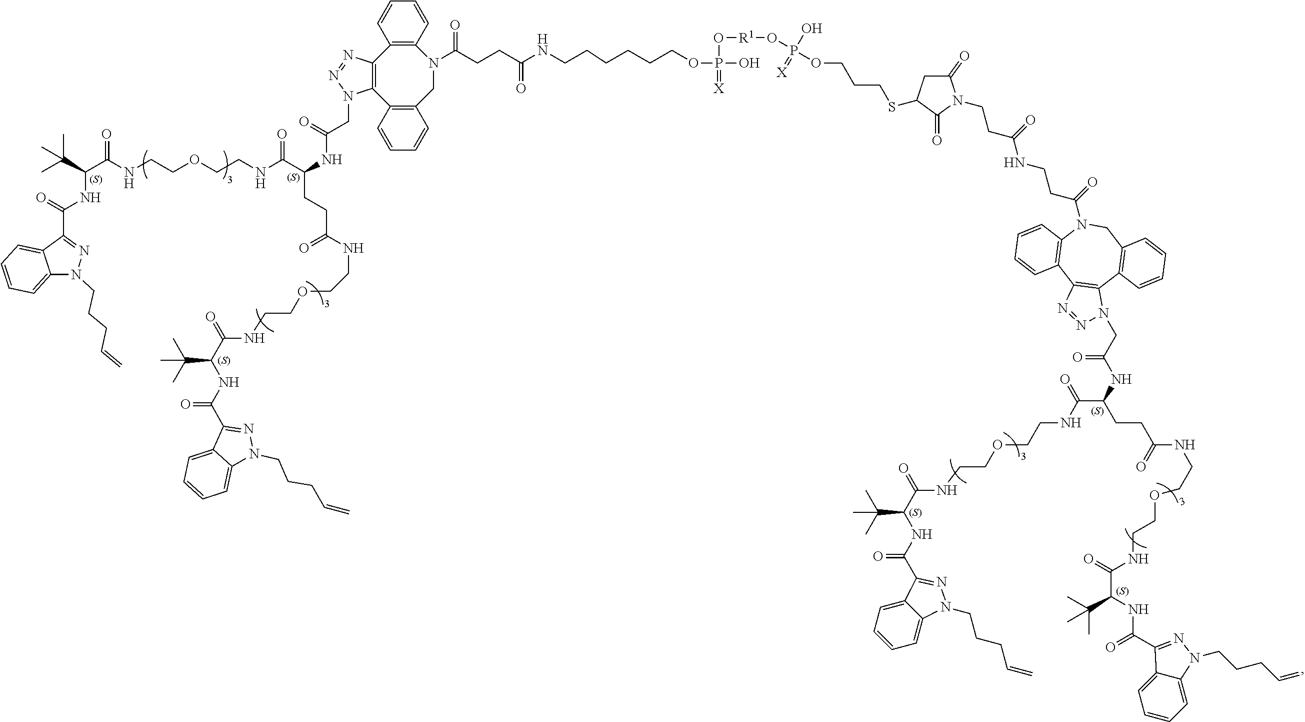 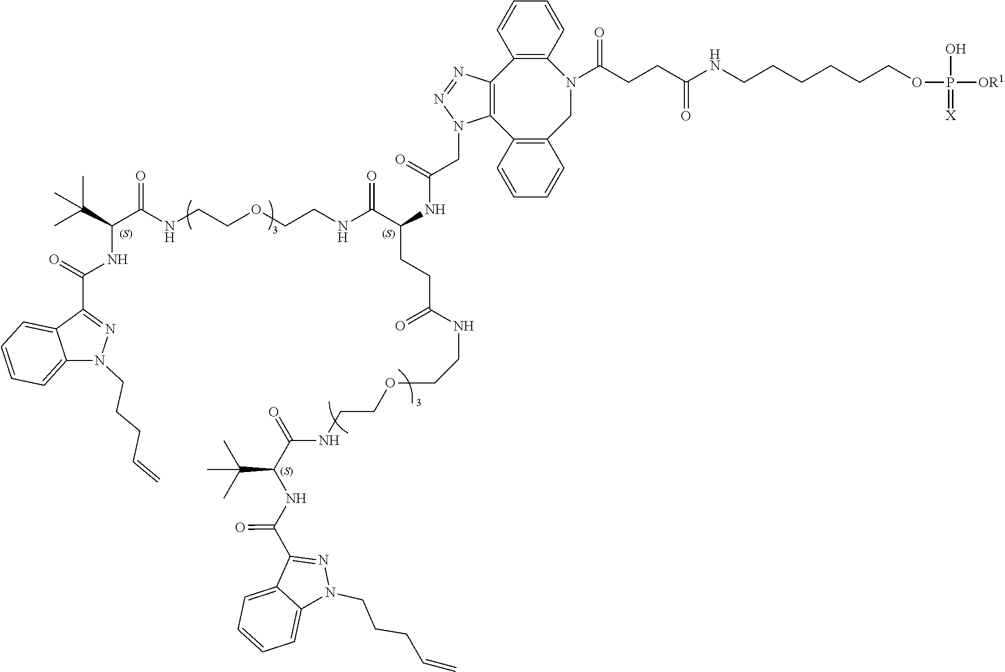 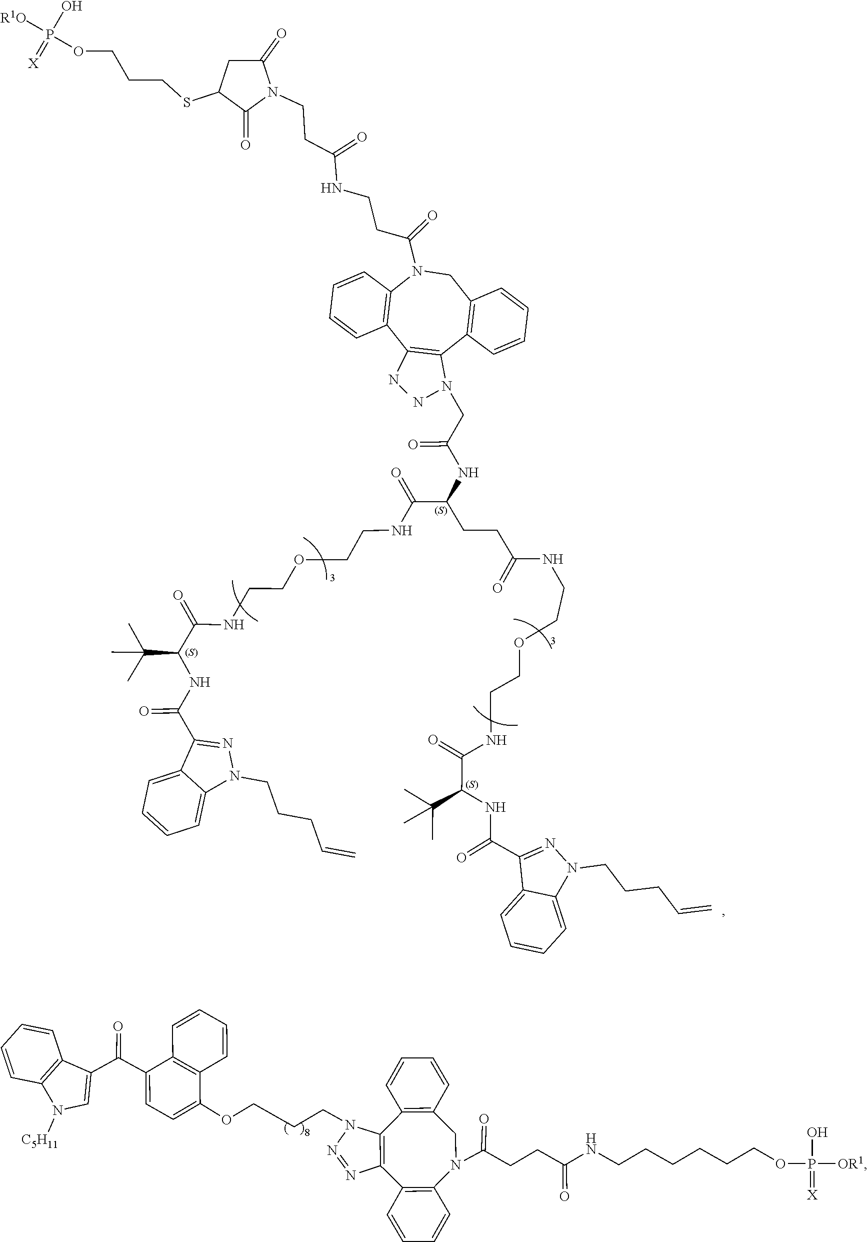 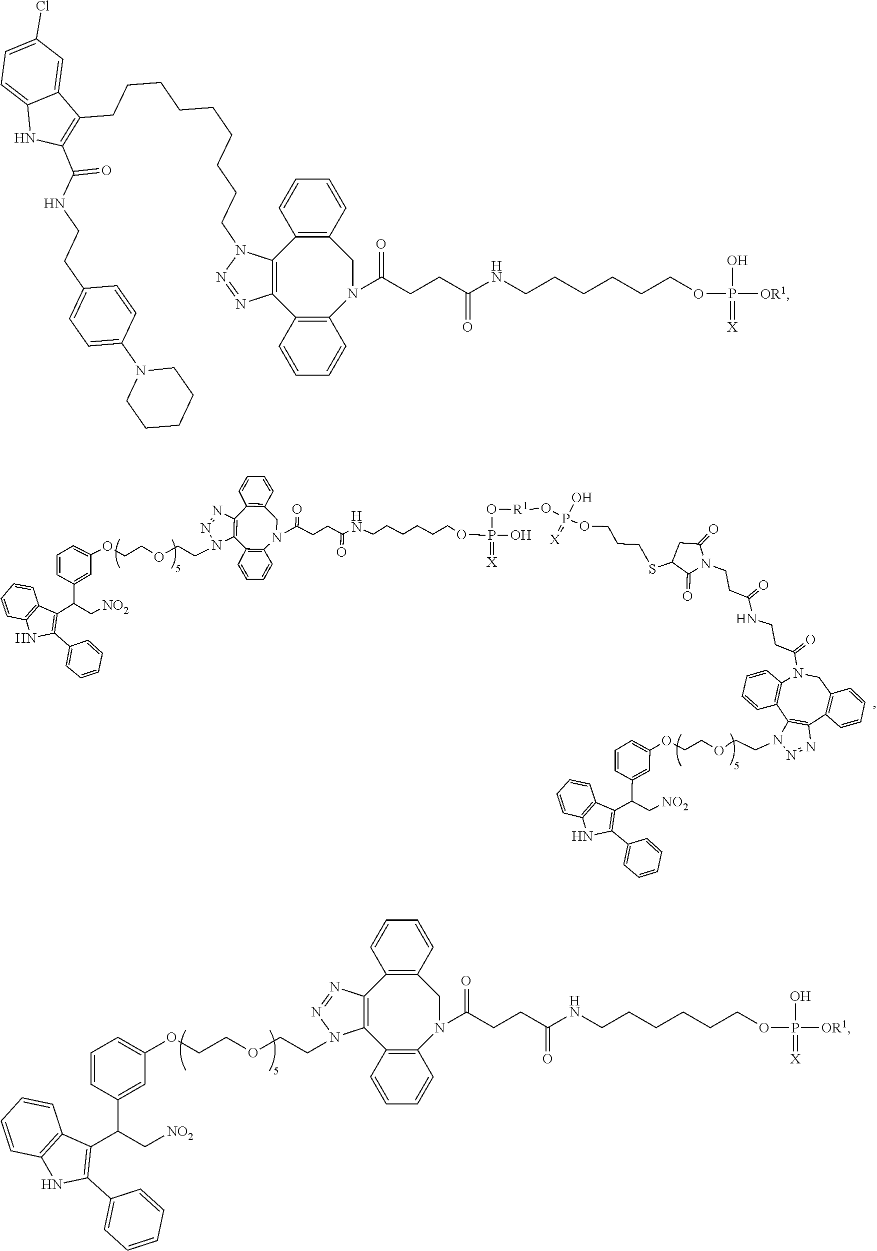 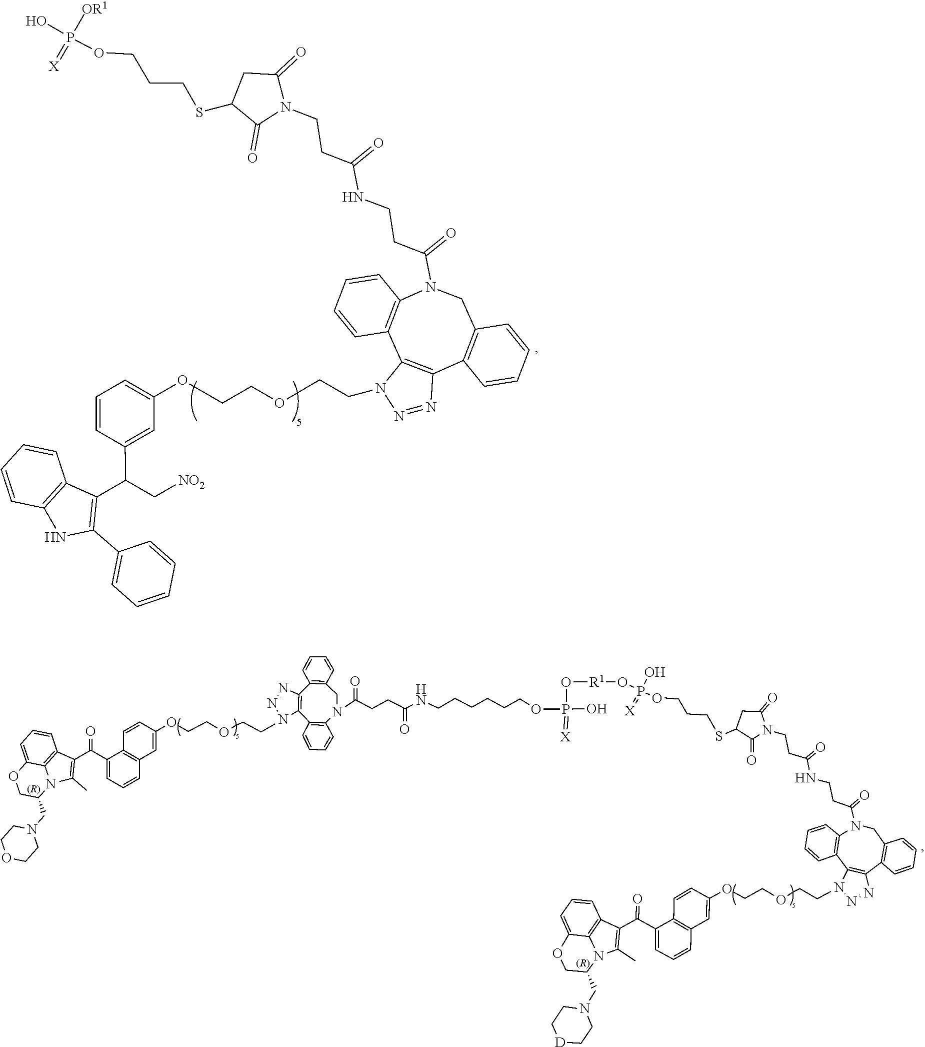 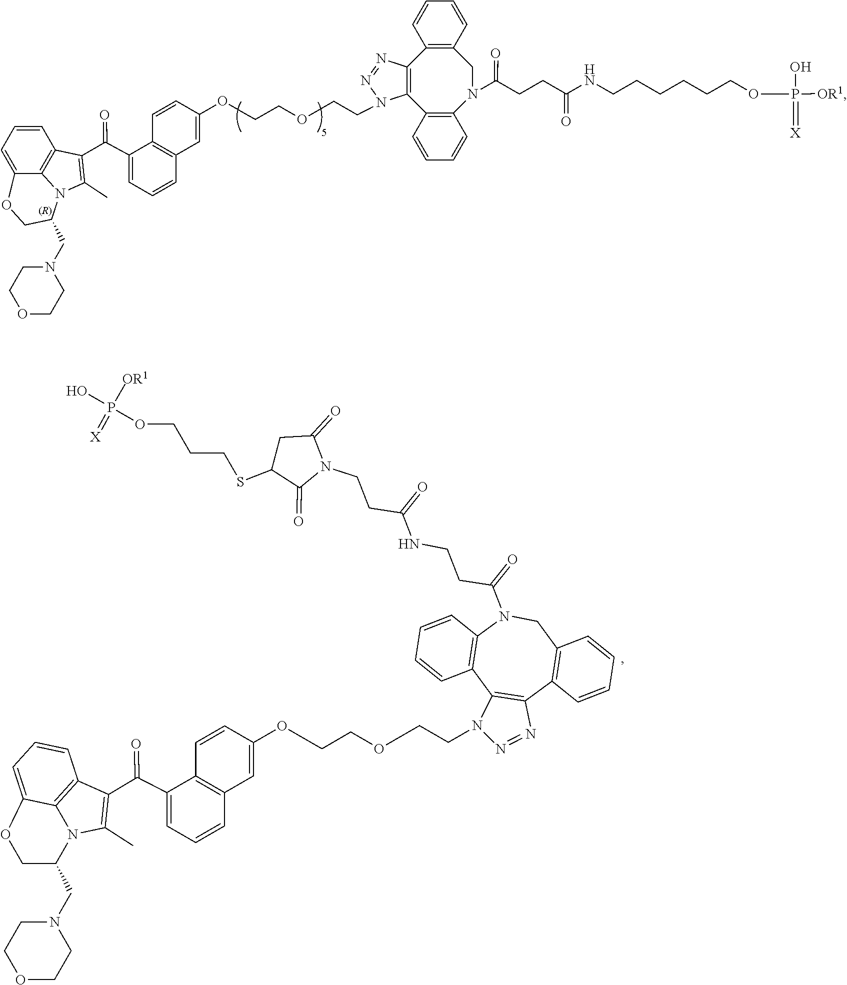 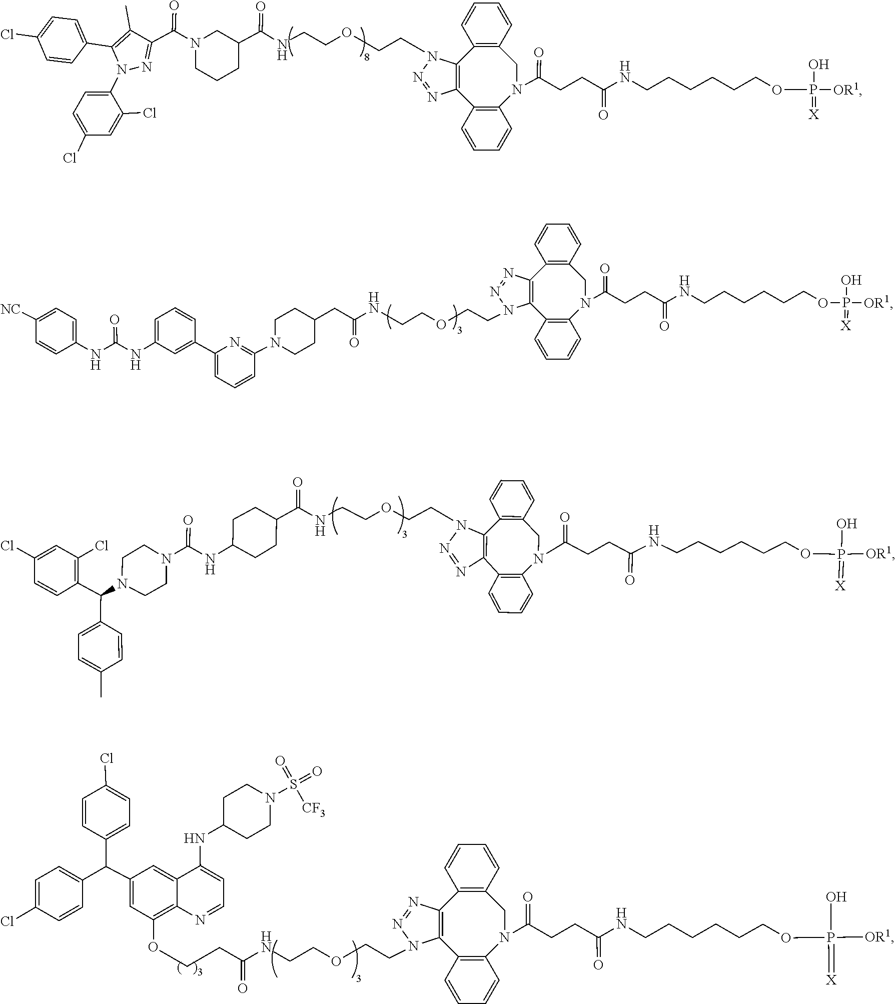 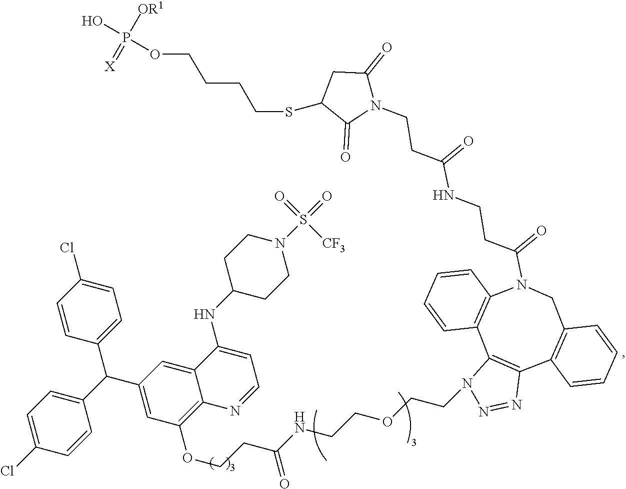 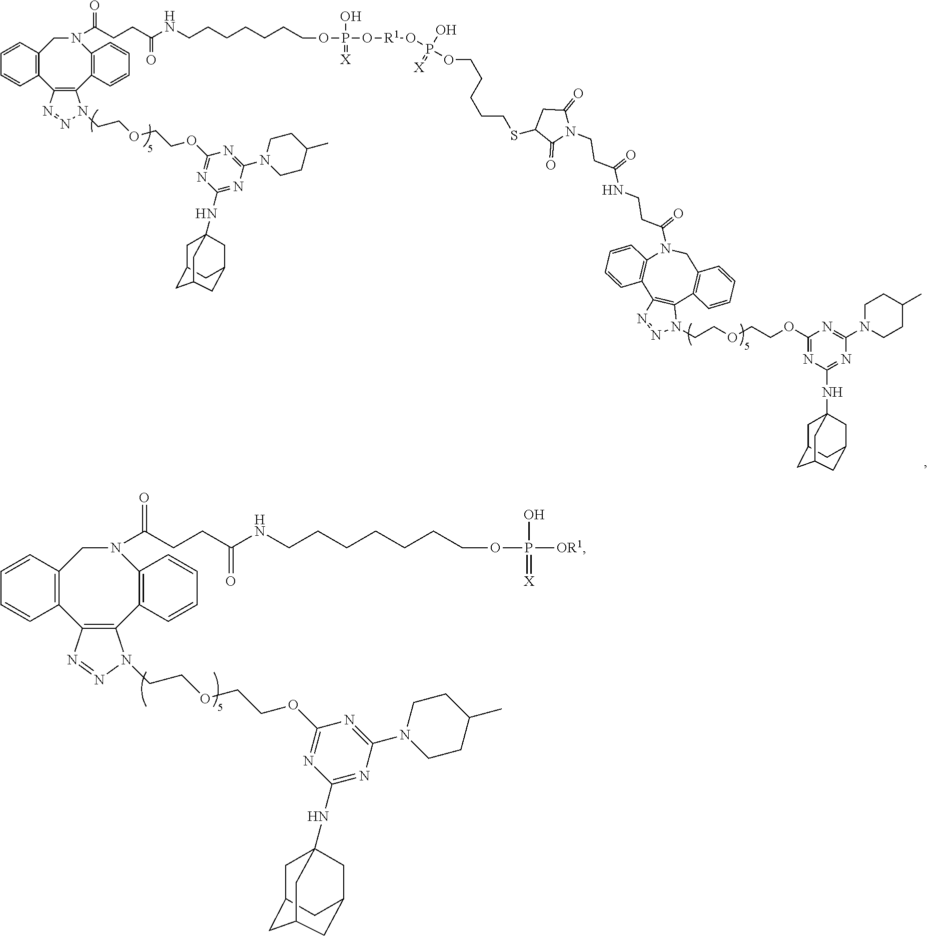 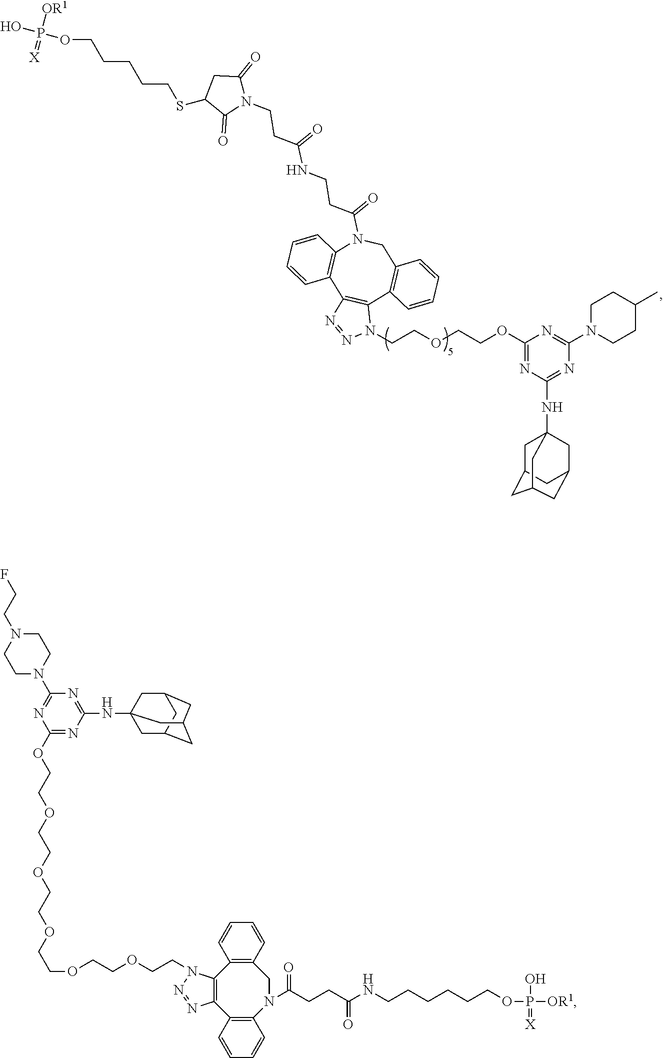 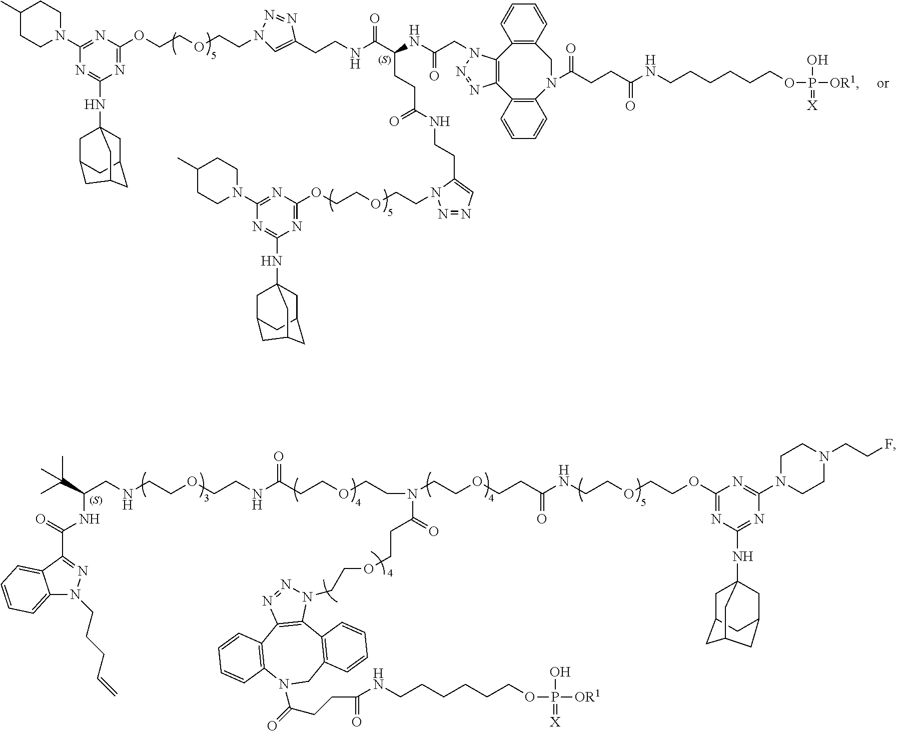
wherein
R¹ and R² each independently comprise one or more oligonucleotides, protecting groups, small molecules, proteins, antibodies, and/or peptides; and
X is O or S.

154. The compound, or a stereoisomer, tautomer, prodrug, or salt thereof, of any one of claims 1-149, wherein the compound comprises the structure: 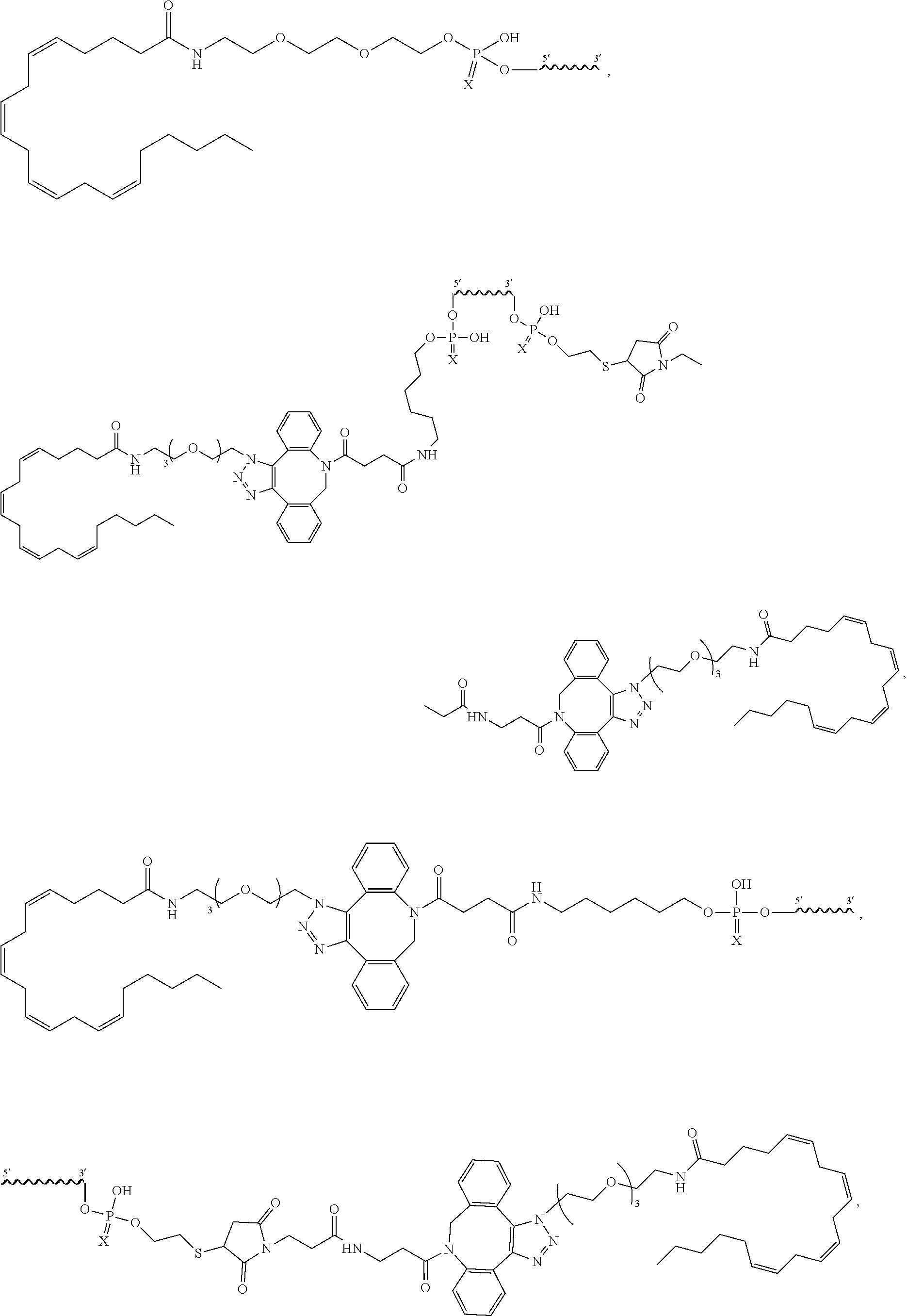 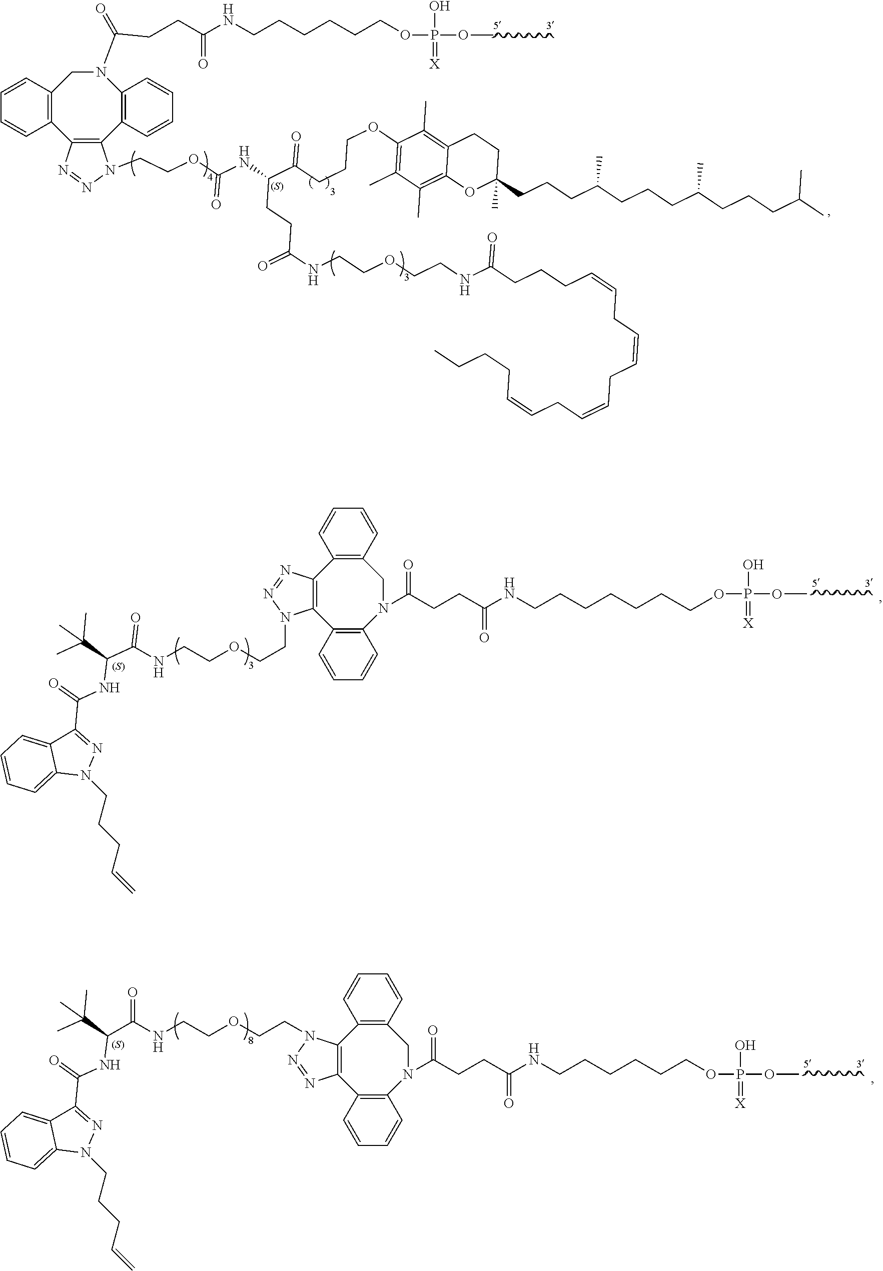 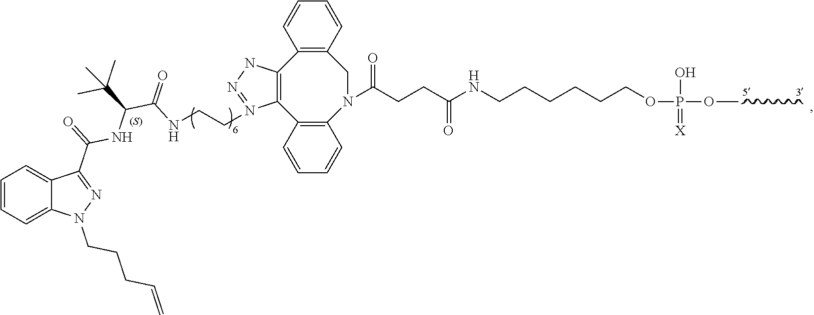 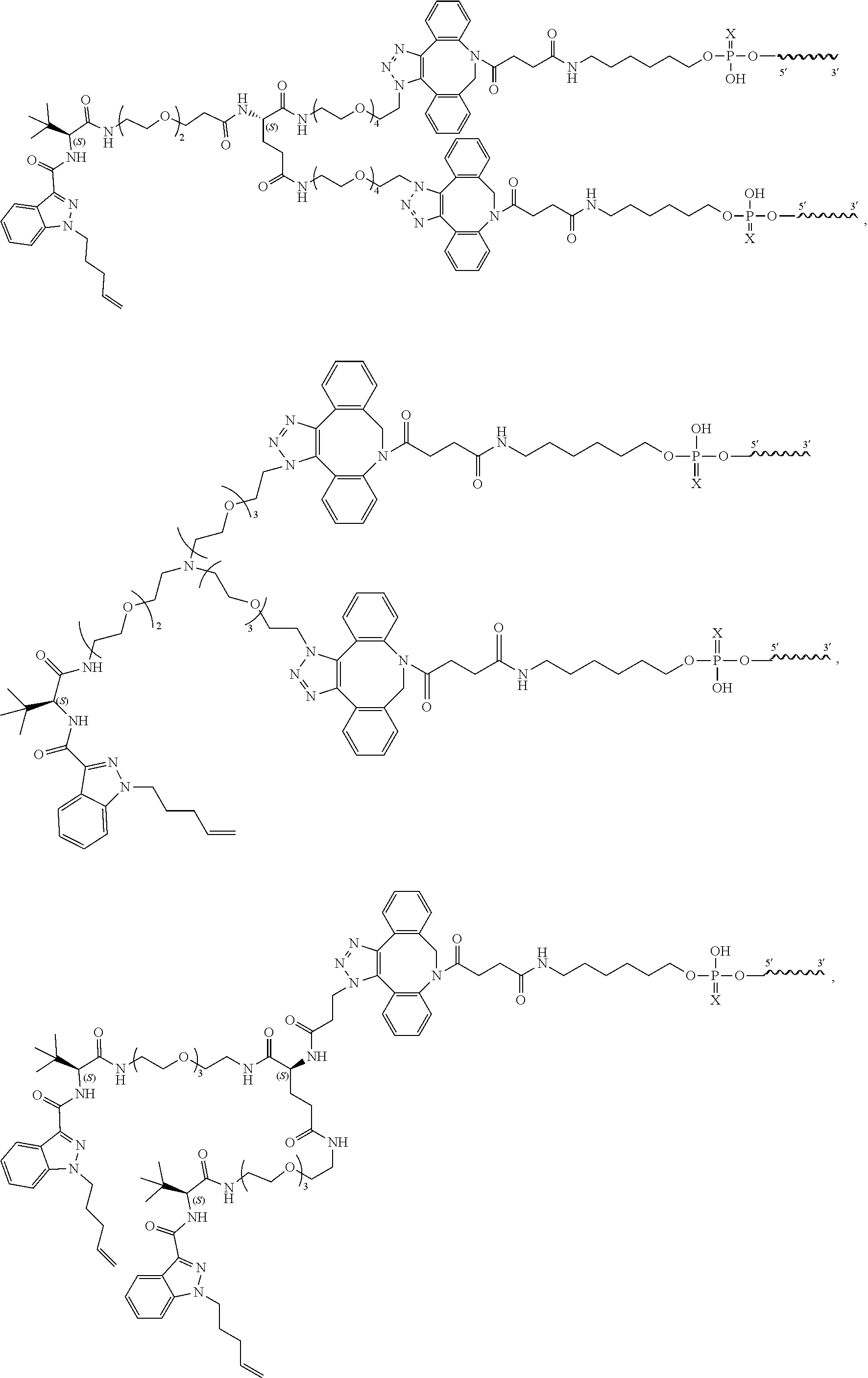 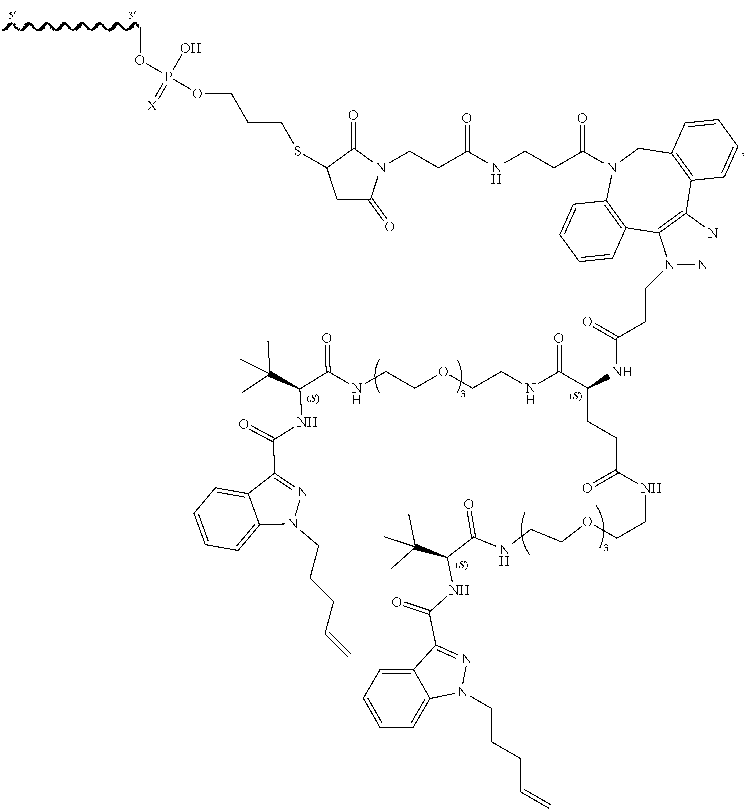 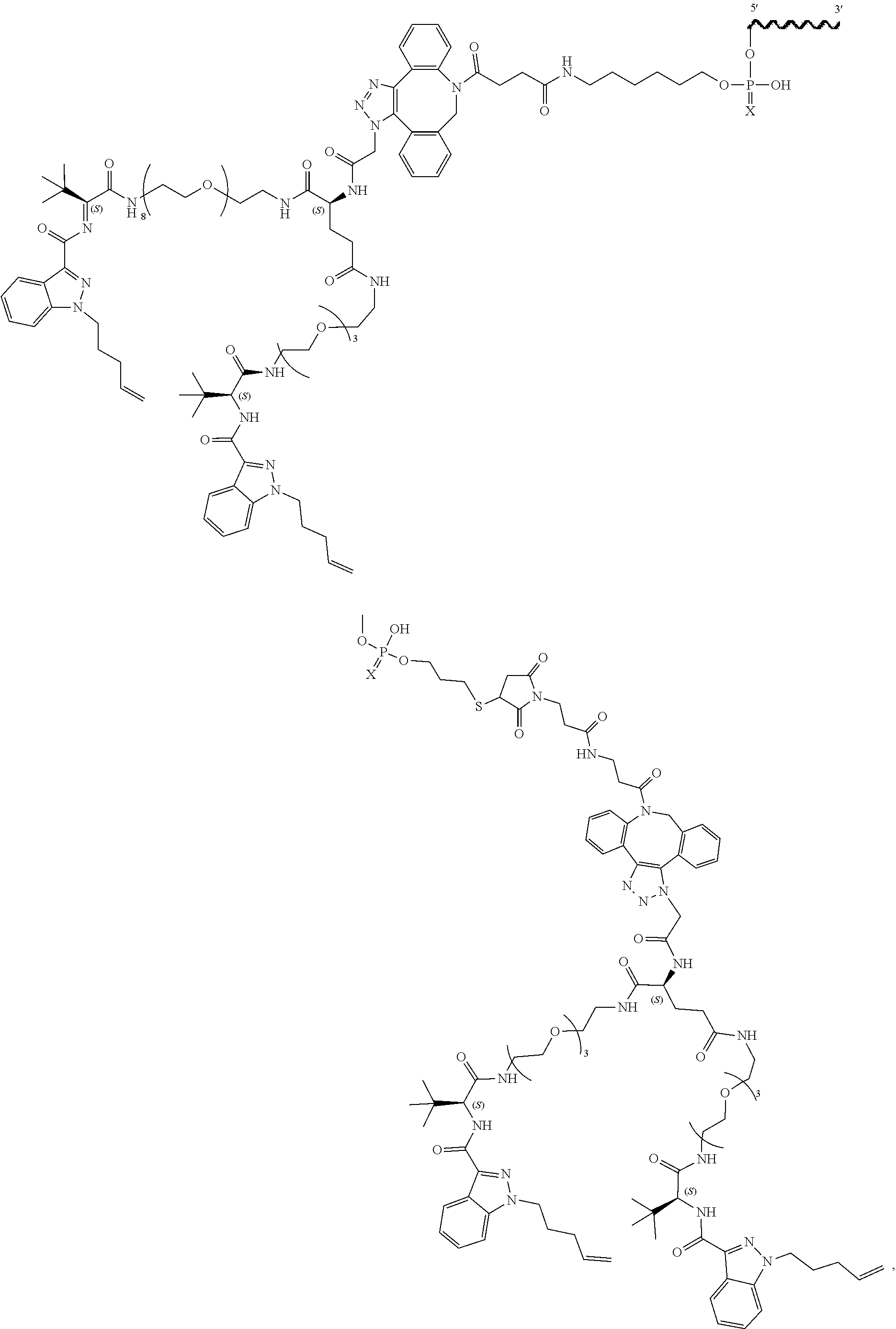 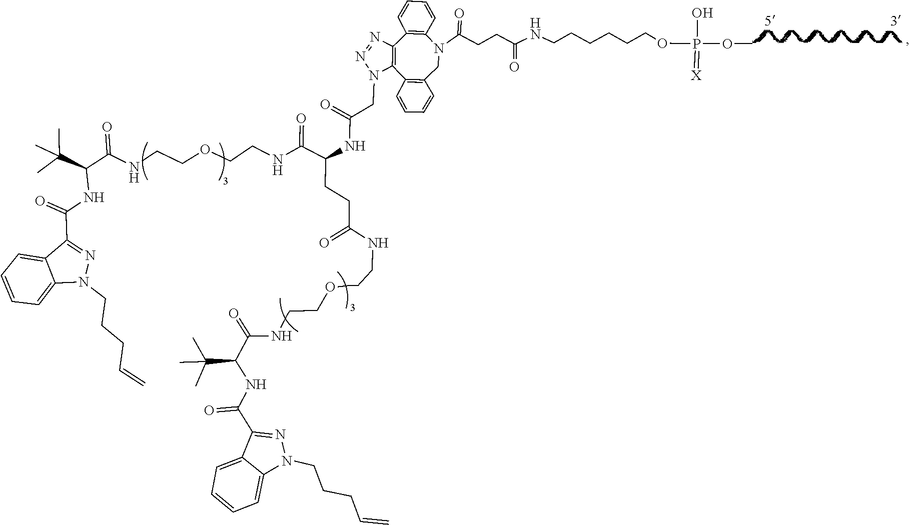 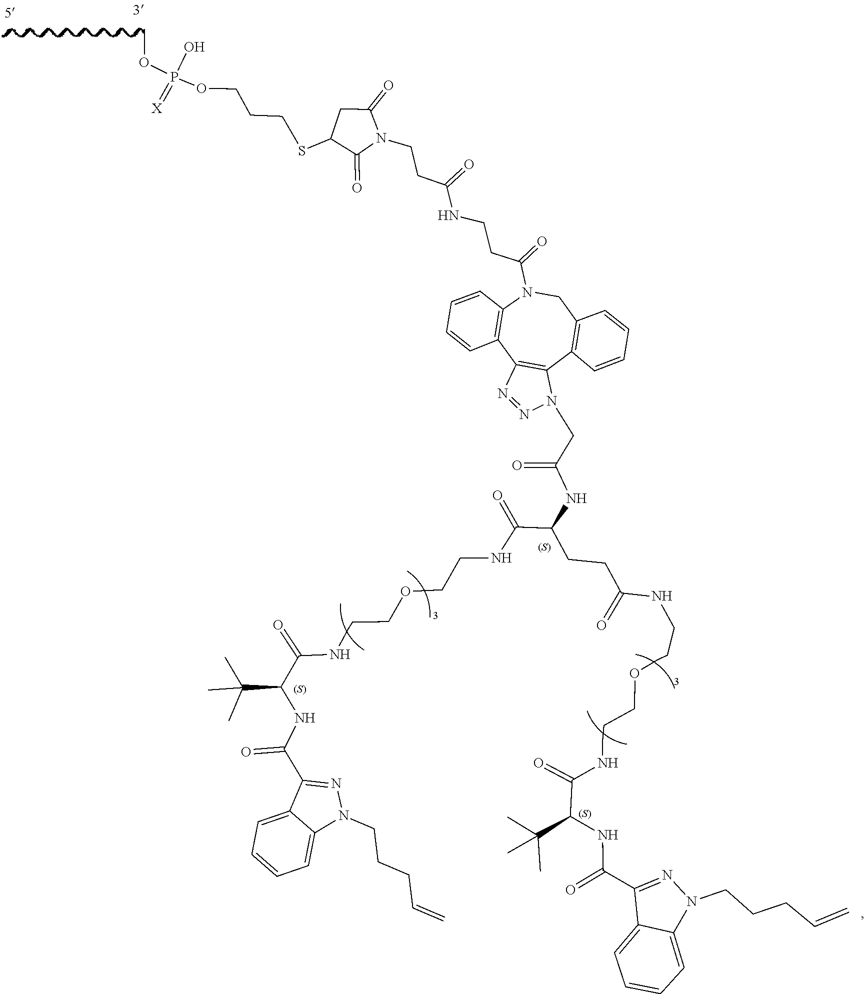 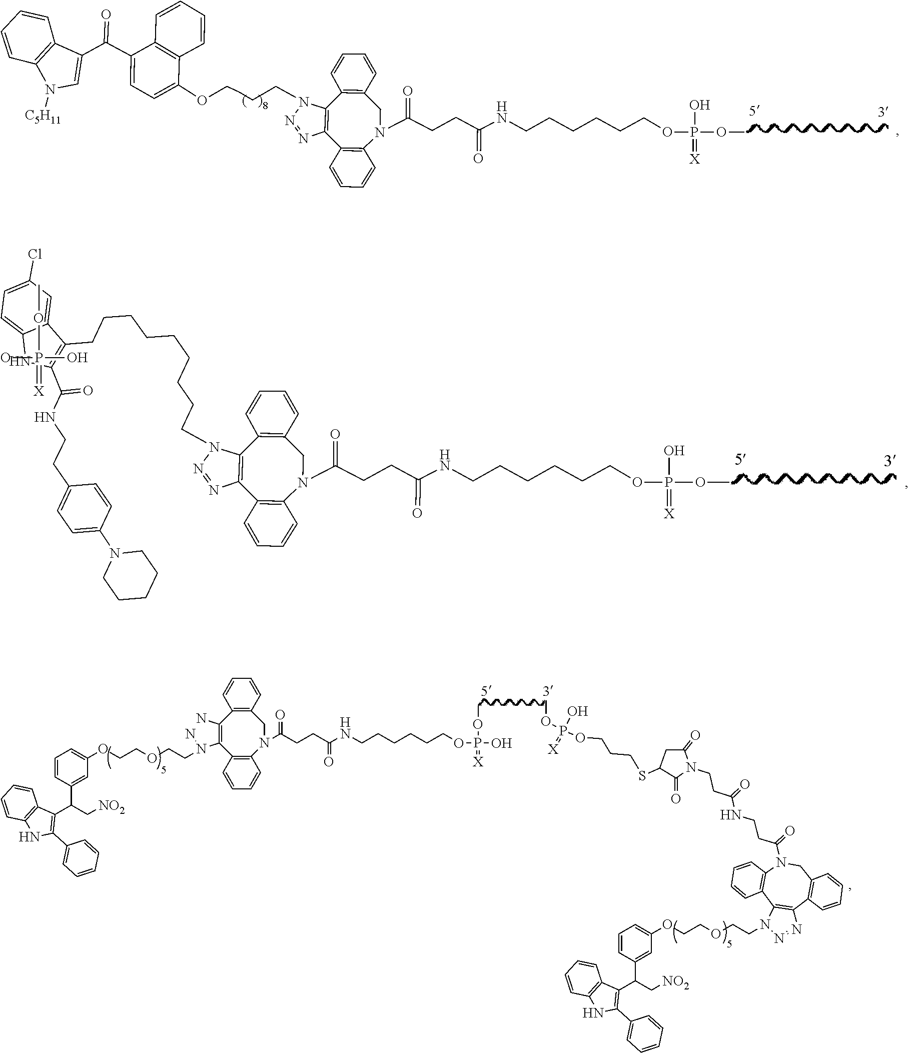 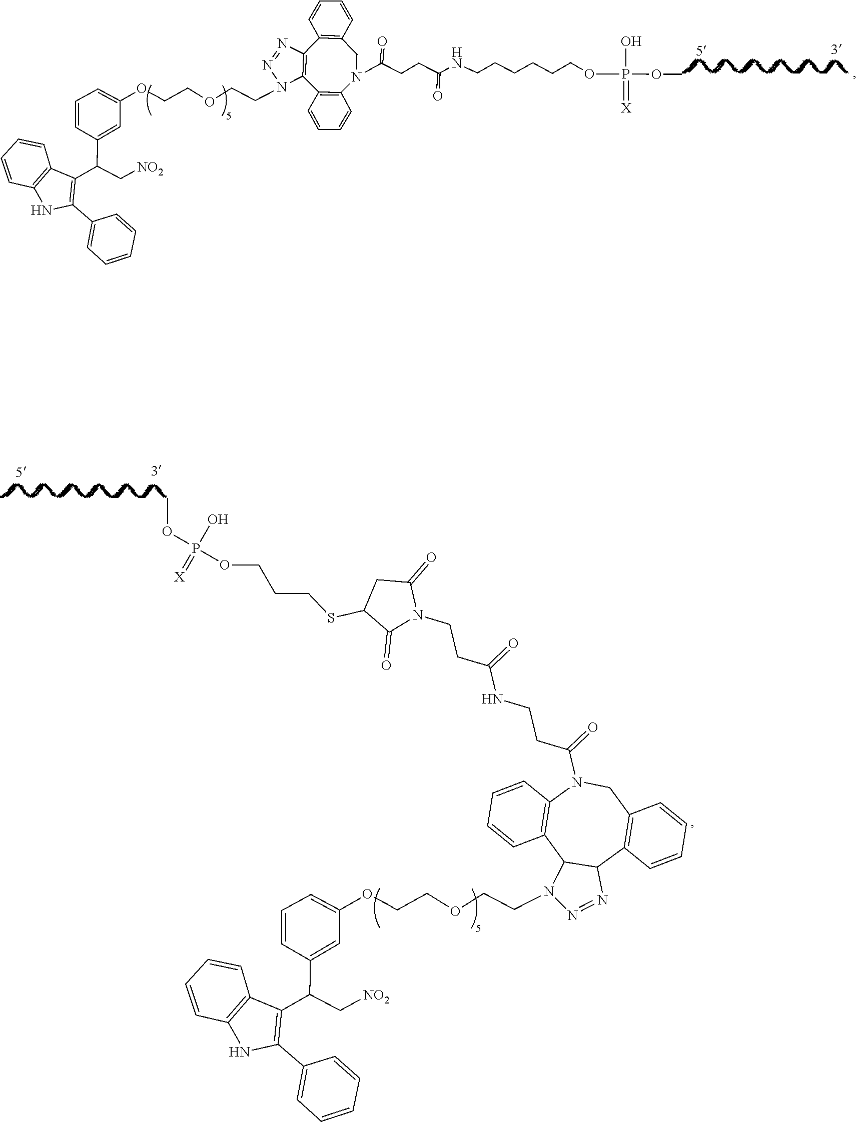 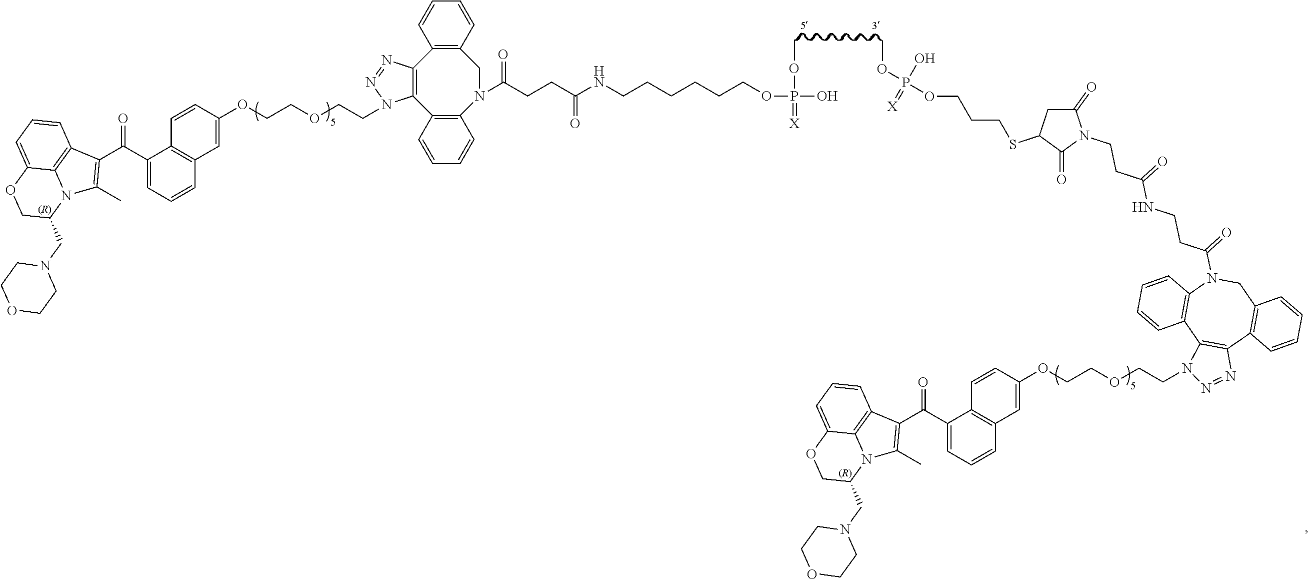 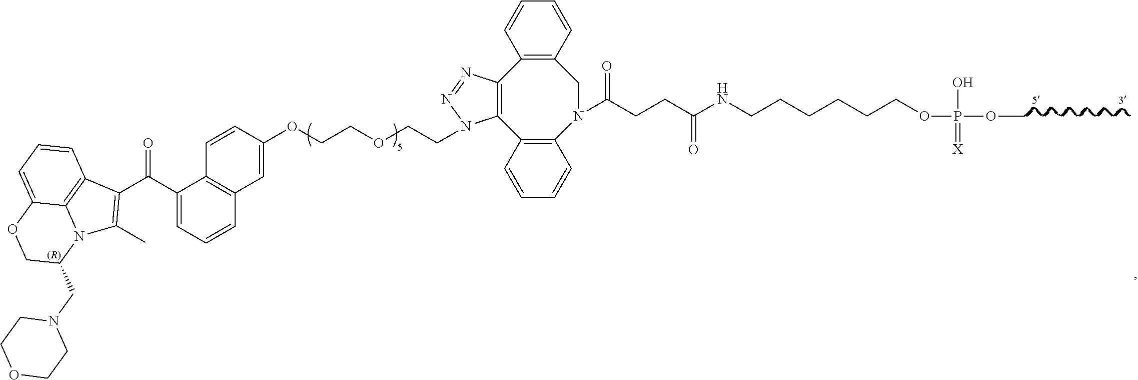 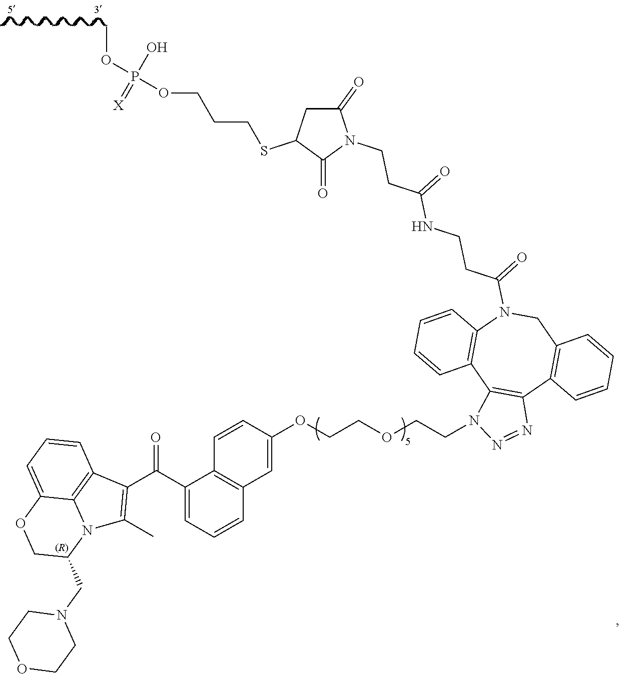 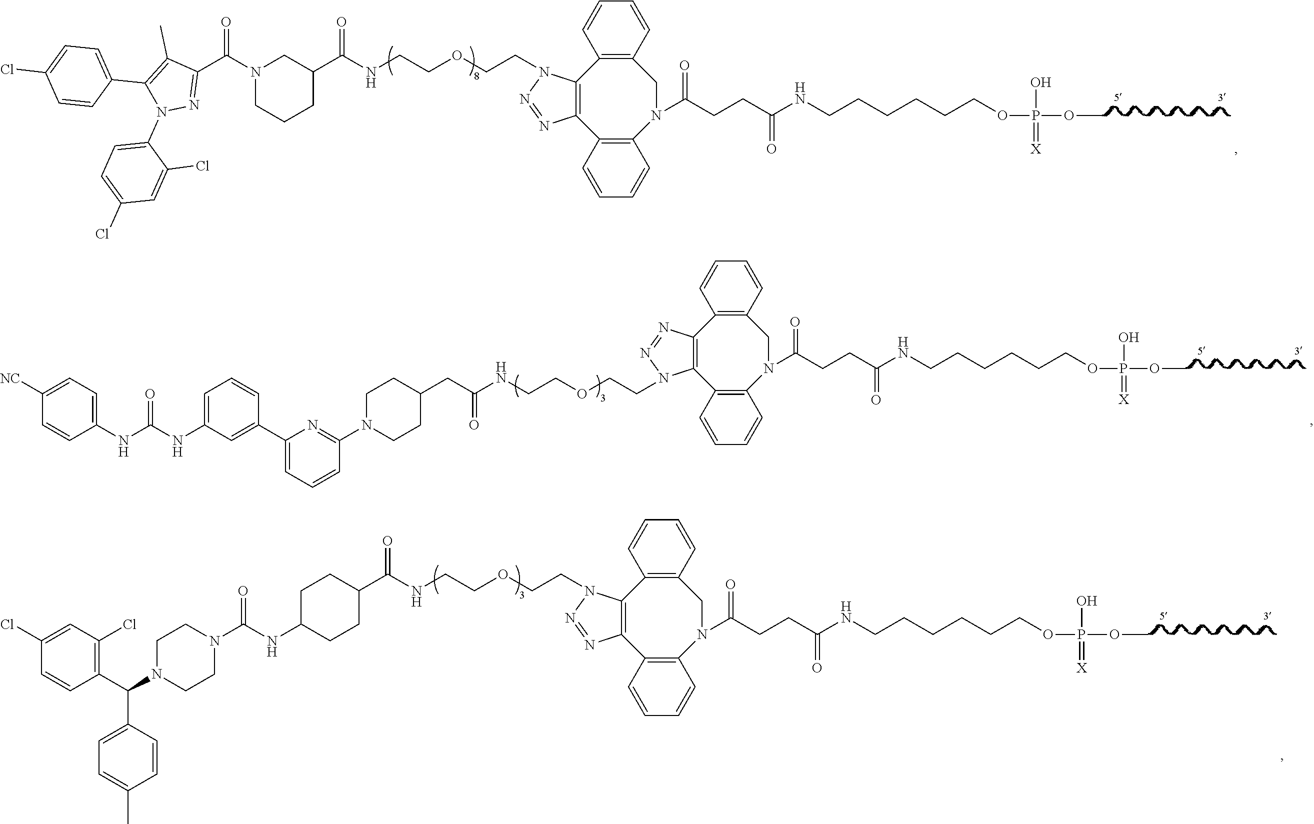 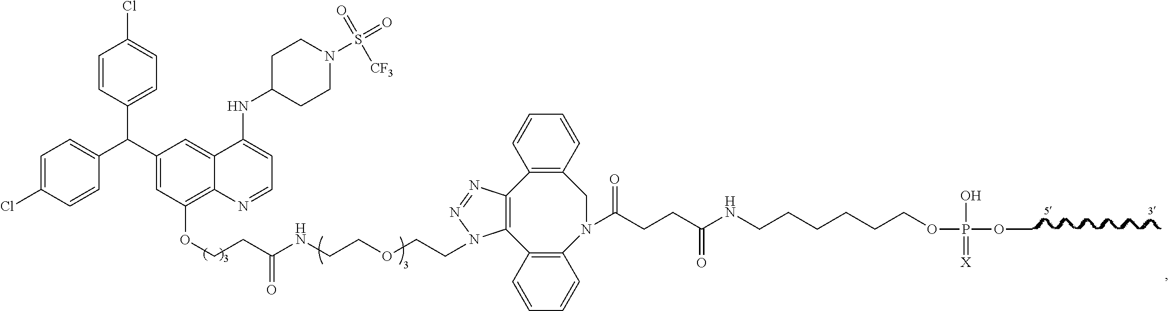 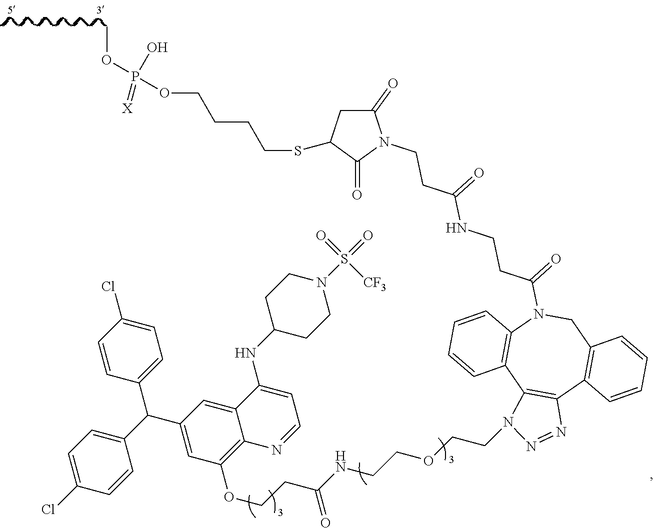 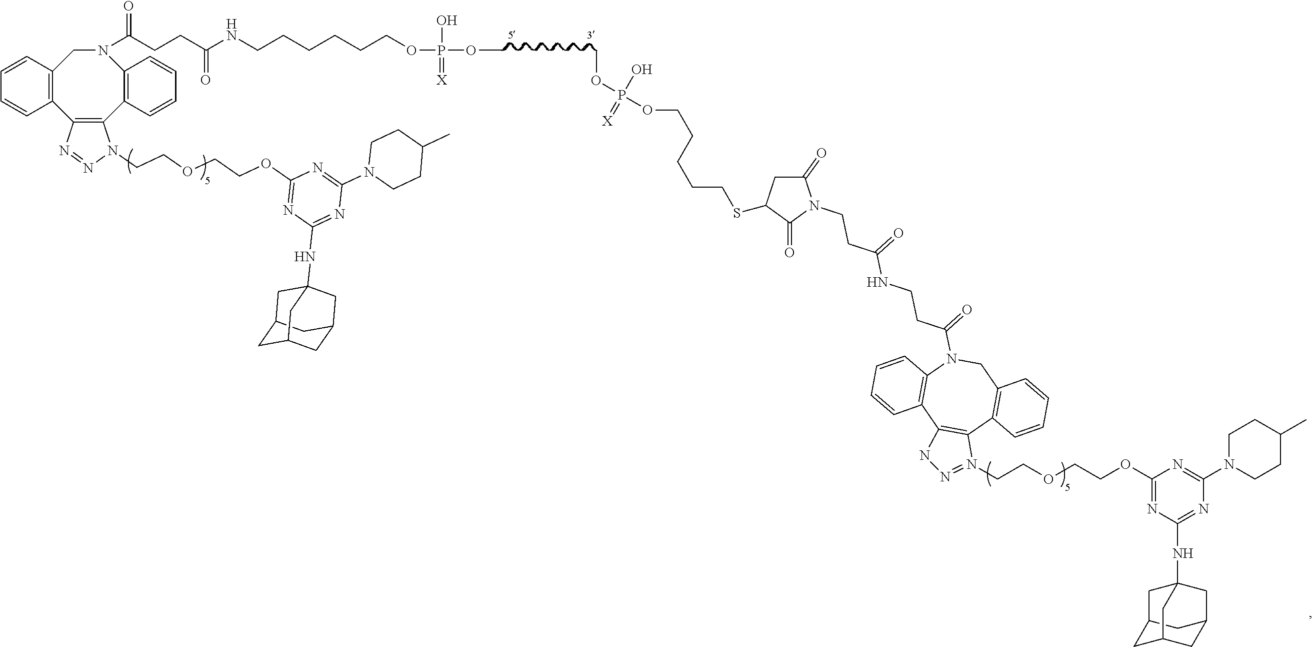 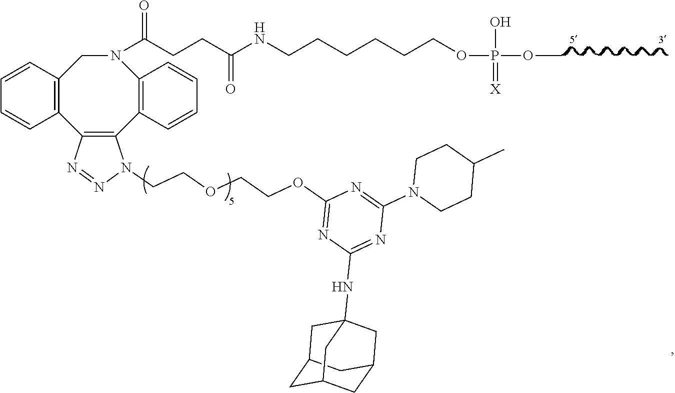 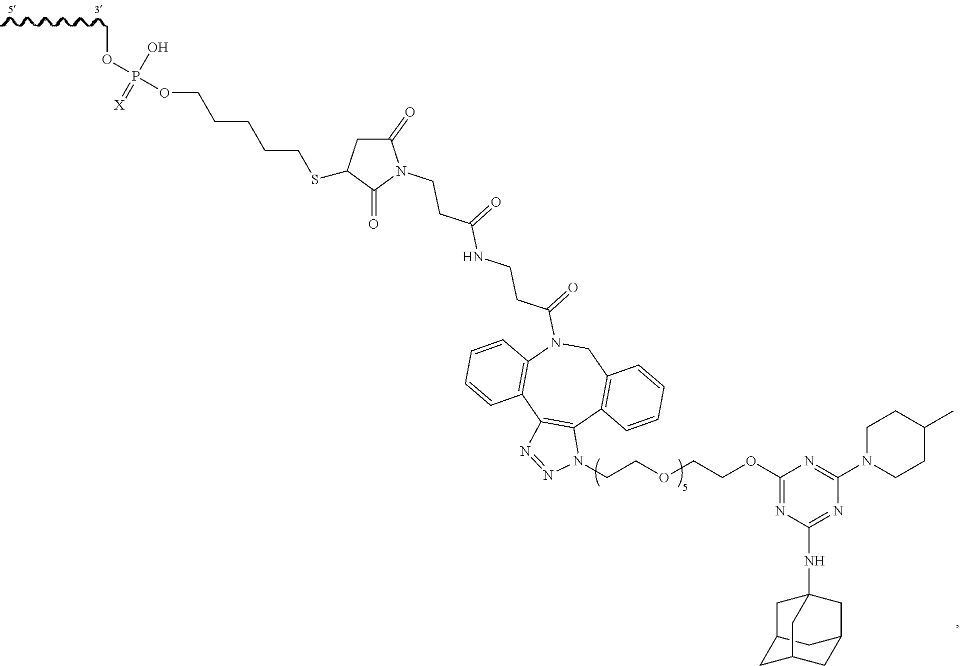 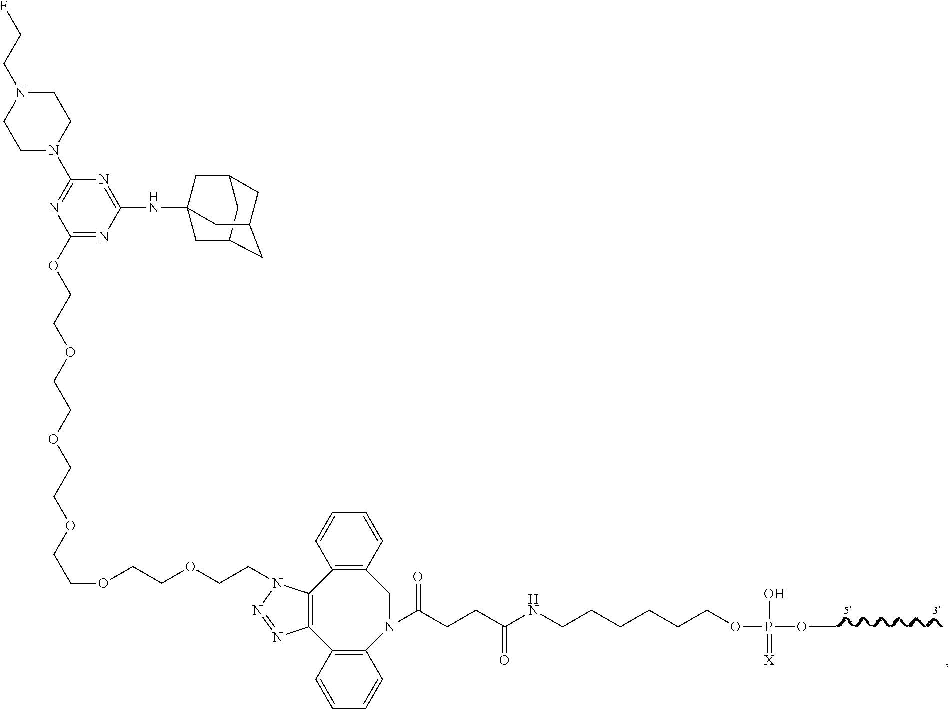 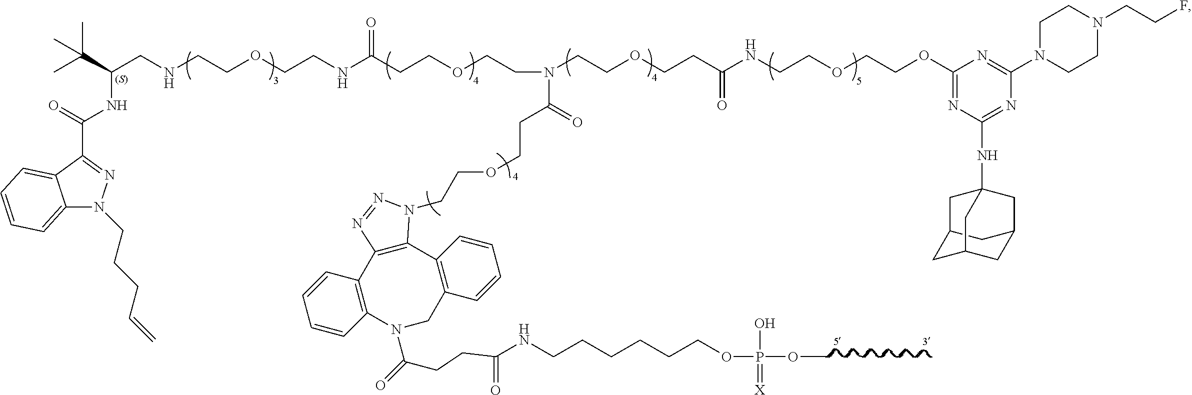
wherein: [symbol]
is an oligonucleotide; and
X is O or S.

155. The compound, or a stereoisomer, tautomer, prodrug, or salt thereof, of any one of claims 112-154, wherein X is S.

156. The compound, or a stereoisomer, tautomer, prodrug, or salt thereof, of any one of claims 112-154, wherein X is O.

157. The compound, or a stereoisomer, tautomer, prodrug, or salt thereof, of any one of claims 1-156, wherein the salt is a potassium salt or a sodium salt.

158. A composition comprising a compound, or a salt thereof, of any one of claims 1-157, and a pharmaceutically acceptable excipient.

159. A method for delivering a therapeutic oligonucleotide to the brain of a subject, comprising administration of a compound, or a salt thereof, of any one of claims 1-157, or a composition of claim 158, to the subject.

160. The method of claim 159, wherein the therapeutic oligonucleotide is delivered to one or more brain regions selected from the group consisting of the striatum, the cerebellum, the brain stem, the hippocampus, the frontal cortex, and the spinal cord.

161. A method for treating or ameliorating a disease, disorder, or symptom thereof in a subject, comprising administration of a compound, or a salt thereof, of any one of claims 1-157, or a composition of claim 158, to the subject.

162. The method of claim 161, wherein the disease, disorder, or symptom thereof is a central nervous system (CNS) disease, disorder, or symptom thereof.

163. The method of claim 161 or 162, wherein the disease, disorder, or symptom thereof is Alzheimer's disease, or a symptom thereof.

164. The method of any one of claims 159-163, wherein the administration is intrathecal administration or intracerebroventricular (ICV) administration.

165. A precursor compound, or a stereoisomer, tautomer, or salt thereof, of any one of structural Formulae (A)-(M): 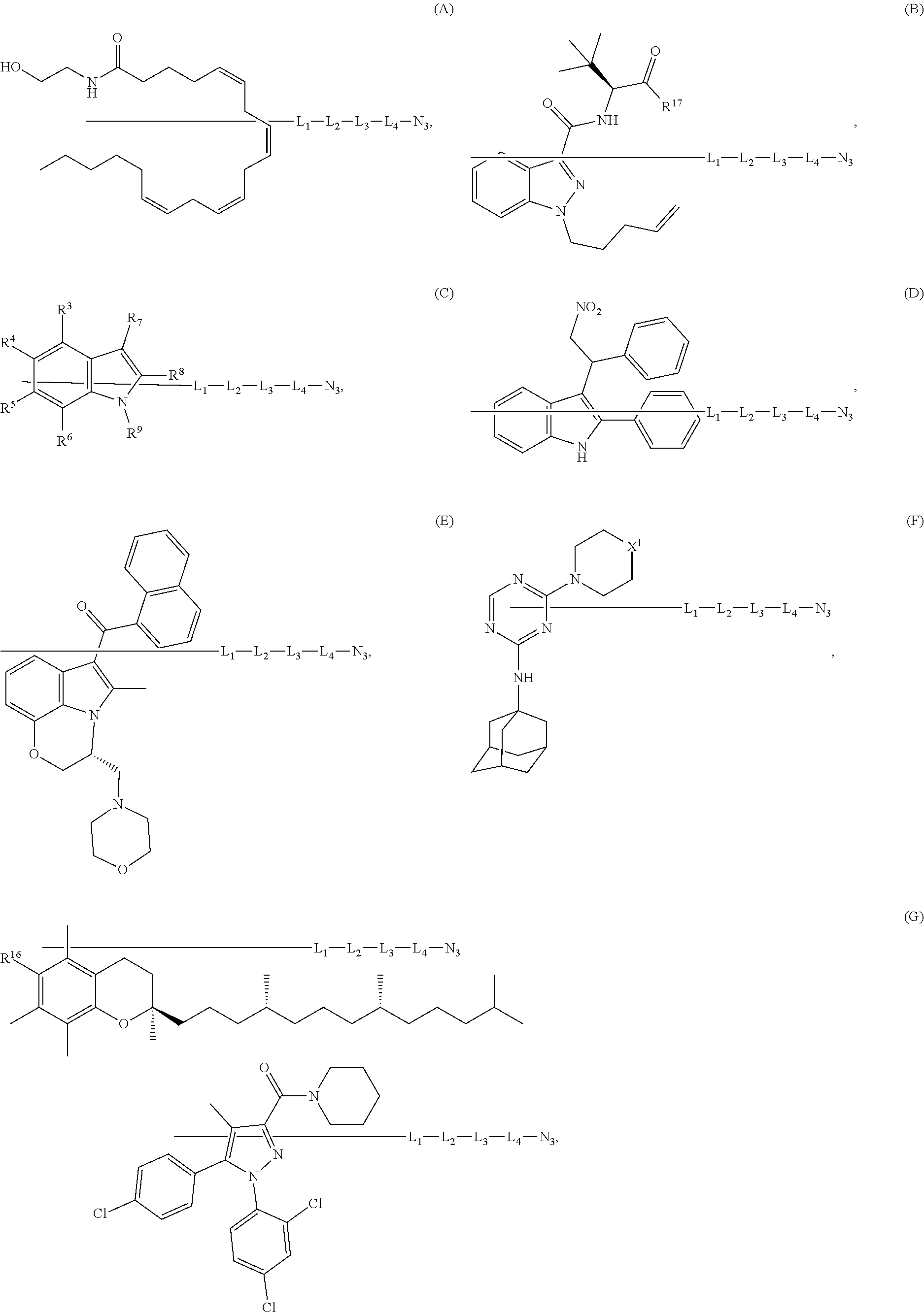 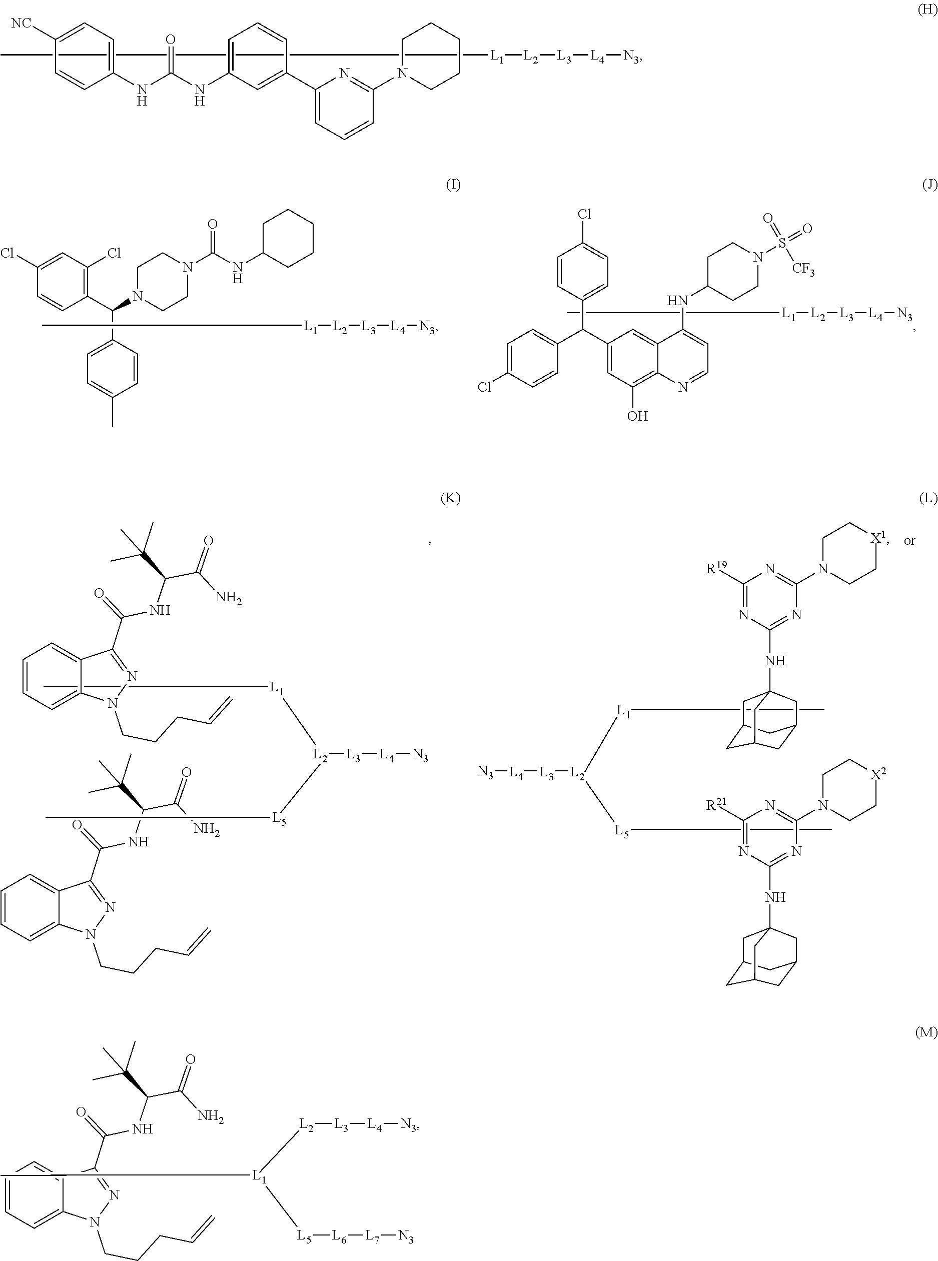
wherein:
each of L₁, L², L³, L⁴, L⁵, L⁶, and L⁷ is independently a linker, bond, or absent;
X¹ is NR¹⁰ or CR¹¹R¹²;
X² is NR¹³ or CR¹⁴R¹⁵;
R³, R⁴, R⁵, R⁶, and R⁸ are each independently hydrogen, halogen, optionally substituted alkyl, optionally substituted heteroalkyl, optionally substituted cycloalkyl, optionally substituted heterocycloalkyl, optionally substituted aryl, or optionally substituted heteroaryl;
R⁹ is hydrogen, optionally substituted alkyl, or optionally substituted heteroalkyl; or R⁶ and R⁹ substituents may be joined together form an optionally substituted heterocycloalkyl or optionally substituted heteroaryl;
R⁷ is hydrogen, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D};
—NR^{7B}C(O)OR^{7D}, —NR^{7B}OR^{7D}, optionally substituted alkyl, optionally substituted heteroalkyl, optionally substituted cycloalkyl, optionally substituted heterocycloalkyl, optionally substituted aryl, or optionally substituted heteroaryl;
R^{7A}, R^{7B}, R^{7C}, R^{7D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{7B} and R^{7C} substituents bonded to the same nitrogen atom may optionally be joined to form a substituted or unsubstituted heterocycloalkyl or substituted or unsubstituted heteroaryl;
n7 is 0, 1, 2, 3, or 4; and
v7 is 1 or 2.
R¹⁰, R¹¹, R¹², R¹, R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, —CN, —N₃, —NO₂, —NR^{16B}R^{16C}, —C(O)R^{16D}, —C(O)OR^{16D}, —C(O)NR^{16B}R^{16C}, —OR^{16A}, —NR^{16B}C(O)R^{16D} optionally substituted alkyl, optionally substituted heteroalkyl, optionally substituted cycloalkyl, optionally substituted heterocycloalkyl, optionally substituted aryl, or optionally substituted heteroaryl;
R¹⁷ is hydrogen, —SOₙ₁₇R^{17A}, —SOᵥ₁₇NR^{17B}R^{17C}, —NHNR^{17B}R^{17C}, —ONR^{17B}R^{17C}, —NHC(O)NHNR^{17B}R^{17C}, —NHC(O)NR^{17B}R^{17C}, —NR^{17B}R^{17C}, —C(O)R^{17D}, —C(O)OR^{17D}, —C(O)NR^{17B}R^{17C}, —OR^{17A}, —NR^{17B}SO₂R^{17A}, —NR^{17B}C(O)R^{17D}, —NR^{17B}C(O)OR^{17D}, —NR^{17B}OR^{17D}, optionally substituted alkyl, optionally substituted heteroalkyl, optionally substituted cycloalkyl, optionally substituted heterocycloalkyl, optionally substituted aryl, or optionally substituted heteroaryl;
R¹⁸ is hydrogen, —SOₙ₁₈R^{18A}, —SOᵥ₁₈NR^{18B}R^{18C}, —NHNR^{18B}R^{18C}, —ONR^{18B}R^{18C}, —NHC(O)NHNR^{18B}R^{18C}, —NHC(O)NR^{18B}R^{18C}, —NR^{18B}R^{18C}, —C(O)R^{18D}, —C(O)OR^{18D}, —C(O)NR^{18B}R^{18C}, —OR^{18A}, —NR^{18B}SO₂R^{18A}, —NR^{18B}C(O)R^{18D};
—NR^{18B}C(O)OR^{1D}, —NR^{18B}OR^{18D}, optionally substituted alkyl, optionally substituted heteroalkyl, optionally substituted cycloalkyl, optionally substituted heterocycloalkyl, optionally substituted aryl, or optionally substituted heteroaryl;
R¹⁹ is hydrogen, —SOₙ₁₉R^{19A}, —SOᵥ₁₉NR^{19B}R^{19C}, —NHNR^{19B}R^{19C}, —ONR^{19B}R^{19C}, —NHC(O)NHNR^{19B}R^{19C}, —NHC(O)NR^{19B}R^{19C}, —NR^{19B}R^{19C}, —C(O)R^{19D}, —C(O)OR^{19D}, —C(O)NR^{19B}R^{19C}, —OR^{19A}, —NR^{19B}SO₂R^{19A}, —NR^{19B}C(O)R^{19D}, —NR^{19B}C(O)OR^{19D}, —NR^{19B}OR^{19D}, optionally substituted alkyl, optionally substituted heteroalkyl, optionally substituted cycloalkyl, optionally substituted heterocycloalkyl, optionally substituted aryl, or optionally substituted heteroaryl;
R²⁰ is hydrogen, —SOₙ₂₀R^{20A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —NR^{20B}R^{20C}, —C(O)R^{20D}, —C(O)OR^{20D}, —C(O)NR^{20B}R^{18C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is independently hydrogen, —SOₙ₂₁R^{21A}, —SOᵥ₂₁NR^{21B}R^{21C}, NHNR^{21B}R^{21C}, —ONR^{21B}R^{21C}, —NHC(O)NHNR^{21B}R^{21C}, —NHC(O)NR^{21B}R^{21C}, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
n19 and n21 are each independently 0, 1, 2, 3, or 4; and
v19 and v21 are each independently 1 or 2;
R^{16A}, R^{16B}, R^{16C}, R^{16D}, R^{17A}, R^{17B}, R^{17C}, R^{17D}, R^{18A}, R^{18B}, R^{18C}, R^{18D}, R^{19A}, R^{19B}, R^{19C}, R^{19D}, R^{20A}, R^{20B}, R^{20C}, R^{20D}, R^{21A}, R^{21B}, R^{21C}, and R^{21D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R^{16B} and R^{16C}; R^{17B} and R^{17D}; R^{18B} and R^{18D}; R^{19B} and R^{19C}; R^{20B} and R^{20C}; and R^{21B} and R^{21C} substituents bonded to the same nitrogen atom may optionally be joined to form a substituted or unsubstituted heterocycloalkyl or substituted or unsubstituted heteroaryl;
n16, n17, n18, n19, n20, and n21 are each independently 0, 1, 2, 3, or 4; and
v16, v17, v18, v19, v20, and v21 are each independently 1 or 2.

166. A method for making a compound, or a stereoisomer, tautomer, prodrug, or salt thereof, of any one of claims 1-157, comprising contacting the precursor compound of claim 165 with a compound of structural Formula (W) and/or (Q): 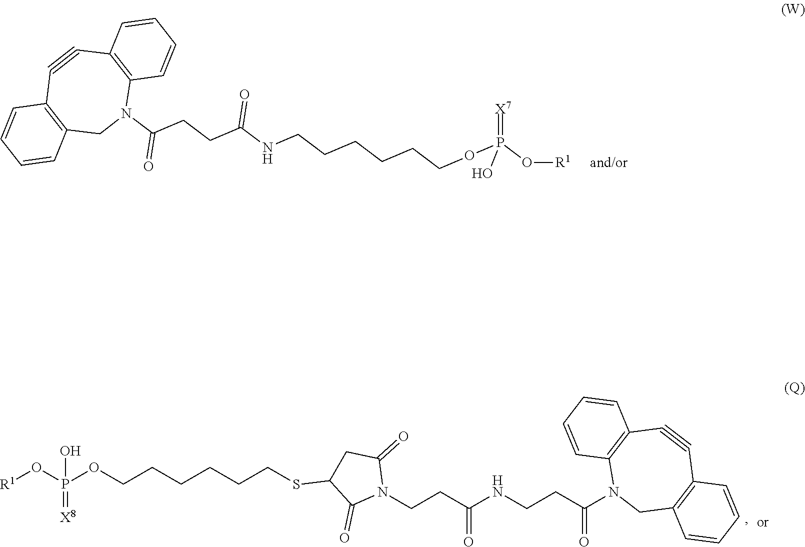
a salt thereof,
wherein X⁷ and X⁸ are each independently O or S.
